(19) United States
(12) Patent Application Publication
(10) Pub. No.: US 2022/0062394 A1
(43) Pub. Date: Mar. 3, 2022

(54) METHODS FOR IDENTIFYING NEOANTIGENS

(71) Applicants: The Broad Institute, Inc., Cambridge, MA (US); Massachusetts Institute of Technology, Cambridge, MA (US); The General Hospital Corporation, Boston, MA (US); Dana-Farber Cancer Institute, Inc., Boston, MA (US)

(72) Inventors: Tamara Ouspenskaia, Cambridge, MA (US); Travis Law, Cambridge, MA (US); Aviv Regev, Cambridge, MA (US); Steven Carr, Cambridge, MA (US); Karl Clauser, Cambridge, MA (US); Susan Klaeger, Cambridge, MA (US); Nir Haochen, Boston, MA (US); Catherine J. Wu, Boston, MA (US); Derin Keskin, Boston, MA (US)

(21) Appl. No.: 17/414,480

(22) PCT Filed: Dec. 12, 2019
(86) PCT No.: PCT/US2019/066104

§ 371 Date: Jun. 16, 2021

Related U.S. Application Data
(60) Provisional application No. 62/780,832, filed on Dec. 17, 2018.
(60) Provisional application No. 62/820,042, filed on Mar. 18, 2019.

(51) Int. Cl. A61K 39/00; G01N 33/68; C12Q 1/6869; A61K 45/06

(52) U.S. Cl. CPC A61K 39/0005; A61K 45/06; C12Q 1/6869; G01N 33/6848

(57) ABSTRACT

Disclosed herein are polypeptides and compositions comprising one or more neoantigens, methods of identifying neoantigens, and methods for preparing a neoantigen for an immunogenic pharmaceutical composition. The neoantigen can be specific to a subject that has a cancer, disease, or other disorder.

### CROSS-REFERENCE TO RELATED APPLICATIONS

This application claims the benefit of U.S. Provisional Application No. 62/780,832, filed Dec. 17, 2018 and U.S. Provisional Application No. 62/820,042 filed Mar. 18, 2019. The entire contents of the above-identified applications are hereby fully incorporated herein by reference.

### STATEMENT REGARDING FEDERALLY SPONSORED RESEARCH

This invention was made with government support under grant numbers CA180922 and CA202820 awarded by the National Institutes of Health. The government has certain rights in the invention.

### TECHNICAL FIELD

The subject matter disclosed herein is generally directed to identification of tumor specific neoantigens and the uses of these neoantigens to produce cancer vaccines.

### BACKGROUND

Cancer vaccines are typically composed of cancer antigens and immunostimulatory molecules (e.g. cytokines or TLR ligands) that work together to induce antigen-specific cytotoxic T cells (CTLs) that recognize and lyse cancer cells. Neoantigen vaccines comprises neoantigens deriving from proteins having cancer-specific mutations within protein-coding sequence. Neoantigens, i.e., mutated peptides from mutated proteins, are presented on MHC-I and recognized by T cells as “foreign,” which mounts an immune response against cancer cells. Neoantigens have been exploited therapeutically to target immune cells against cancer cells.

There are multiple approaches using neoantigens in cancer immunotherapy. For example, a subject's own dendritic cells can be matured and loaded ex vivo with the subject's neoantigens and infused back to activate the subject's T cells. (Science, Vol. 348, Issue 6236, pp. 803-808). A subject's T cells can also be activated and expanded ex vivo in the presence of neoantigen peptides. (Tran et al., Cancer immunotherapy based on mutation-specific CD4+ T cells in a patient with epithelial cancer, Science, Vol. 344, Issue 6184, pp. 641-645, 9 May 2014; Stevanovic et al., Landscape of immunogenic tumor antigens in successful immunotherapy of virally induced epithelial cancer, Science, Apr. 14, 2016, 356(6334):200-205). Finally, patients can be immunized with therapeutic neoantigen vaccines to trigger neoantigen-specific T cell response. This approach has been tested at Dana Farber by Catherine Wu and colleagues. Six melanoma patients were effectively immunized. (Ott et al., An immunogenic personal neoantigen vaccine or patients with melanoma, Nature 2017 Jul. 13; 547(7662):217-221). The vaccine induced the expansion and activation of neoantigen-specific T cells, demonstrating that neoantigen vaccines are a promising therapeutic avenue. In these approaches, the current methods for identifying neoantigens were by using whole exome sequencing (WES) of annotated protein-coding genomic regions, which comprises approximately 2% of the genome. WES works sufficiently well for cancers with abundant somatic mutations, like melanoma. However, for cancers with low mutation burden that are still immunogenic, the current approach of using whole exome sequencing to sample only annotated protein-coding regions of the genome would be frequently insufficient to identify actionable neoantigens. Thus, there is a need for improved tools and methods for identifying neoantigens for making an immunogenic pharmaceutical composition.

Citation or identification of any document in this application is not an admission that such a document is available as prior art to the present invention.

### SUMMARY

In certain example embodiments, polypeptides comprising one or more neoantigens are provided herein, which can be selected from any of Tables 1-3D. In embodiments, the polypeptide comprises 2 or more neoantigenes, which can be linked together directly, or with any of the linkers disclosed herein. In particular embodiments, the polypeptide comprises a T cell enhancer amino acid sequence.

T cell enhancer amino acids may be selected from the group consisting of an invariant chain; a leader sequence of tissue-type plasminogen activator; a PEST sequence, a cyclin destruction box; a ubiquitination signal; and a SUMOylation signal. Compositions comprising the polypeptides and/or vector systems are also provided.

In an aspect, the composition further comprises at least one modulator of a checkpoint molecule or an immunomodulator, or a nucleic acid encoding the modulator or immunomodulator, or a vector comprising the nucleic acid encoding the modulator or immunomodulator for use in preventing or treating a proliferative disease in a subject, which may be an agonist of a tumor necrosis factor receptor superfamily member, preferably of CD27, CD40, OX40, GITR, or CD137; and/or an antagonist of PD-1, PD-L1, CD274, A2AR, B7-H3, B7-H4, BTLA<CTLA-4, IDO, KIR, LAG3, TIM-3, VISTA, or an antagonist of a B7-CD28 superfamily member, preferably of CD28 or ICOS or an antagonist of a ligand thereof; and/or the immunomodulator is a T cell growth factor, preferably IL-2, IL-12, or IL-15.

In an aspect, the composition can further comprise one or more adjuvants.

Methods of identifying neoantigens are also provided, and can comprise the steps of performing Ribosomal profiling (Ribo-seq) on a sample or set of samples; generating a novel untranslated open reading frame (nuORF) database comprising predicted nuORFs by conducting hierarchical ORF prediction on the Ribo-seq data generated; generating a final set of neoantigens by searching the nuORF database for predicted nuORFs in the nuORF database matching data in a MHC I immunopeptidome data set, the identified presented nuORFs comprising the final neoantigen set. The method may further comprise searching an annotated proteome database for ORFs in the annotated proteome database matching data in the MHC I immunopeptidome dataset. The method may also further comprise selecting presented nuORFs identified in the nuORF database but not the annotated proteome database to generate the final set of neoantigens. In an aspect, the MHC I immunopeptidome data is obtained on biological sampled from a subject to be treated. In an embodiment, the immunopeptidome data is mass spectroscopy data.

Methods of identifying subject-specific T cell receptor (TCR) pairs suitable for subject-specific cancer therapy are also provided, the method comprising: isolating from the subject a population comprising T cells; determining by single cell sequencing the sequences encoding the TCR pairs on individual cells in the population isolated; transfecting or transducing T cell lines deficient in endogenous TCRs with the sequences encoding individual TCR pairs determined; using the T cell lines from the method step to assay binding of the subject specific TCR pairs to subject specific neoepitopes and selecting the TCR pairs that bind to subject-specific neoepitopes. In embodiments, of the method, the subject specific neoepitopes are expressed on HLA molecules on a cell. In an aspect, the cells are antigen presenting cells. In an aspect, the binding of the T cells to the neoepitopes activates a reporter gene. In an embodiment, the neoepitopes are present in tetramers. The neoepitopes can be nuORFs.

Samples or set of samples for the methods used herein may be subject-specific, tissue specific, disorder-specific, or disease-specific. The disease or disorder can be genetic, pathogenic or cancer.

Methods of generating antibodies care provided, comprising administering the neoantigen compositions disclosed herein to the immune system, or a component thereof, of the subject. In an aspect, the immune system component is a B cell.

Methods of treatment are disclosed comprising administering the neoantigen compositions herein to a subject with a disease. Methods for identifying patient specific neoantigens are provided comprising performing Ribosomal profiling (Ribo-seq) on a patient specific tumor sample and a non-tumor sample from the patient; and identifying nuORFs specific for the tumor sample. The method may further comprise identifying T cells obtained from the patient specific for one or more of the identified neoantigens. In an aspect, the method can comprise a step of expanding T cells specific for the one or more of the identified neoantigens.

T cells specific for a neoantigen identified by the methods disclosed herein. In an aspect, the T cell is obtained from PBMCs from the patient.

The one or more neoantigens, or a polynucleotide encoding the one or more neoantigens disclosed herein can be provided on one or more vectors. A vector system comprising one or more expression vectors are disclosed herein, including wherein each expression vector is selected from the group consisting of a plasmid, a cosmid, a RNA, a RNA formulated in a particle, a self-amplifying RNA (SAM), a SAM formulated in a particle, or a viral vector. Viral vectors can in some embodiments be an alpha virus vector, a Venezuelan equine encephalitis (VEE) virus vector, a sindbis virus vector, a semliki forest virus vector, a simian or human cytomegalovirus vector, a lymphocyte choriomenigitis virus vector, a retroviral vector, a lentiviral vector, an adenovirus vector, or combination thereof. In an aspect, the vector comprises a self-amplifying RNA vector or an adenovirus vector. Methods can include administering the compositions disclosed herein at one or more timepoints to a subject. Administering the composition can, in an aspect, generate a T cell response.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1: (a) depicts the representation of different ORF types in the databases and a comparison of the number of peptides across databases; (b) shows the proteins (peptides) identified in MHC-I immunopeptidome using mass spectometry.

FIG. 2: (a) shows that nuORFs are shorter than canonical ORFs; (b) shows that nuORFs are translated at slightly lower levels than annotated ORFs; (c) shows that nuORFs have comparable MS peptide score; (d) shows that nuORFs have comparable delta-forward reverse score; (e) shows that nuORFs have comparable backbone cleavage score; (f) shows that nuORF peptide motifs are similar to annotated proteins.

FIG. 3: (a) shows that fewer nuORFs found in total proteome; (b) shows that nuORFs detected in the whole proteome are shorter than nuORFs detected on MHC-I; (c) shows a comparison of nuORF lengths found in MHC-I immunopeptidome vs. whole proteome.

FIG. 4: (a) depicts 39% of variants fall within nuORFs translated in a particular patient; (b) shows that Ribo-seq allows to prioritize neoantigens by restricting them to highly translated, where the mutant variant is supported by reads, and with high MHC-I binding affinity; (c) illustrates neoantigen shortlists selected based on Ribo-seq was able to prioritize those neoantigens that elicited T cell response and activation with patient's cells from the clinical trial.

FIG. 5 shows that potential neoantigens are commonly discovered with WES, coupled with RNA-seq. WES covers pre-determined annotated exons, based on the probes included in the assay, which is about 2% of the genome. RNA-seq used to gauge expression levels. MHC-I binding affinity is computationally established.

FIG. 6 shows that ribosome profiling can identify translated open reading frames. (a) Ribosome profiling procedure. (b) Sequence analysis

FIG. 7 shows that codon resolution can allow the identification of novel ORFs.

FIG. 8 shows examples of ORFs identified by Ribo-seq.

FIG. 9 shows the three different categories of neoantigens identified by Ribo-seq: annotated ORFs containing mutations; de novo inter/intra-genic ORFs not expressed in healthy tissues (category 1); and unannotated ORFs, normally expressed in healthy tissues but with acquired mutations (category 2).

FIG. 10 illustrates the experimental design for identifying neoantigens.

FIG. 11 shows HLA peptide detection and identification for full proteome/tryptic samples and HLA samples.

FIG. 12 demonstrates the vast amount of possible ORFs in the transcriptome.

FIG. 13A-13B shows the different databases that can be used. 13A) Peptide identification from unannotated ORFs and useful databases. 13B) Reads by PanSample database.

FIG. 14 shows that incorporating Ribo-seq based ORF predictions reduces the search space.

FIG. 15 shows data dependent acquisition—LC/MS/MS.

FIG. 16 shows comparisons between the different databases—RNA-seq v. Ribo-seq v. B721 vs. PanSample.

FIG. 17 shows peptides from hundreds of unannotated ORFs presented on MHC-I.

FIG. 18 shows that unannotated ORFs detected by mass spectrometry are shorter and translated at slightly lower levels.

FIG. 19 shows that peptides from unannotated ORFs are comparable to peptides from canonical ORFs.

FIG. 20 shows that short ORFs are preferentially presented on MHC-I vs. whole proteome.

FIG. 21 shows somatic variants identified in cancer samples.

FIG. 22 illustrates incorporation of somatic variants into predicted translated ORFs.

FIG. 23 shows Pearson correlation of translation (TPM) among samples.

FIG. 24 shows differentially translated ORFs across samples/tissues.

FIG. 25A-25F. (25A) MHC I immunopeptidome identification. (25B) Distribution of peptides from nuORFs. (25C) Distribution of MHC I-displayed peptides from nuORFs within 5′ UTRs. (25D) Distribution of MHC I-displayed peptides from nuORFs within but out of frame relative to an annotated ORF. (25E) Detection of annotated ORFs and nuORFs across multiple HLA alleles. (25F) Proportion of annotated ORFs and nuORFs identified.

FIG. 26A-26G. (26A) Predicted ORFS across the annotated transcriptome. (26B) Cell types in PanSample database. (26C) Tree structure of ORF prediction pipeline. (26E) Distribution of peptides from nuORFs. (26F) Peptide identification (annotated and unannotated) by database. (26G) Peptide spectrum by database.

FIG. 27A-27C. (27A) Length of nuORF-derived proteins and canonical proteins that contribute peptides to MHC I presentation. (27B) Translation levels of MHC I detected annotated ORFs and nuORFs. (27C) MS detection scorse of MHC I peptides of annotated ORFs and nuORFs. (27D) Annotated and 5′ uORF ARAF peptides presented on MHC I. (27E) Comparison of MHC I-bound peptide sequences of annotated ORFs and nuORFs. (27F) Correlation of MHC I-bound peptides of annotated ORFs and nuORFs.

FIG. 28A-28D. (28A) Translation levels of MHC I detected annotated ORFs and nuORFs. (28B) MS detection scores of MHC I detected annotated ORFs and nuORFs. (28C) Backbone cleavage scores of MHC I detected annotated ORFs and nuORFs. (28D) Rank 1-Rank 2 Scores of MHC I detected annotated ORFs and nuORFs.

FIG. 29A-29F. (29A) Peptides derived from nuORF proteins in cancer cells. (29B) Proportion of nuORF peptides in MHC I samples. (29C) Proportions of annotated ORFs and nuORFs in CLL and melanoma. (29D) Translation levels of canonical and nuORFs differentially translated across cancer and healthy tissues. (29E) Length of nuORF-derived proteins and canonical proteins in cancer cells. (29F) Observation rates of canonical ORFs and nuORFs types.

FIG. 30A-30F. (30A) Distribution of nuORF types in cancer cells. (30B) Ratios of nuORFs types in cancer cells. (30C) Proportions of nuORF types in cancer cells. (30D) Overlap of MHC I alleles of nuORF-derived peptides in CLL and glioblastoma samples compared to B721.221 cells. (30E) Pearson correlation of translation levels of nuORFs between samples. (30F) Differentially translated canonical and nuORFs across samples.

FIG. 31A-31D. (31A) Coverage by WES and WGS of nuORF types. (31B) Prioritization of somatic variants. (31C) Neoantigens from canonical ORFs and nuORFs in melanoma and glioblastoma. (31D) Validated neoantigen peptides in melanoma and glioblastoma.

FIG. 32A-32E: Thousands of nuORFs from Ribo-seq are translated and contribute peptides to the MHC I immunopeptidome. 32A. Ribo-seq approach for nuORF database and MHC IIP and LC-MS/MS for peptide identification. 32B. Sample read contribution to nuORFdb shown as percent of Ribo-seq reads contributed by each tissue type. 32C. Hierarchical ORF prediction approach. ORFs are predicted independently at three levels from reads in each sample (leaves), multiple samples of the same tissue (branches) and all samples (root). 32D. Hierarchical prediction increases power while maintaining tissue specificity. Left: Pooling reads across samples allows ORF detection (bottom track) even when each sample alone will have insufficient reads (top two tracks). Right: Predicting in individual samples (top two tracks) detects overlapping ORFs. 32E. Diverse nuORFs contribute to the MHC I immunopeptidome. Top: Percent of MS/MS spectra mapped to nuORF peptides (red) identified in the MHC I immunopeptidome of 92 HLA mono-allelic B721.221 samples. Bottom: The number of detected nuORFs (x axis) of various types (y axis).

FIG. 33A-33M—nuORFs peptides in the MHC I immunopeptidome are comparable to those from annotated ORFs. 33A-33G. Comparable features of nuORFs and annotated peptides. 33A. LC-MS/MS Spectrum Mill identification score (y axis) for nuORF (pink) and annotated (grey) peptides (mean scores: 11.7 nuORF, 11.4 annotated; 2.4% to 3.8% increase, 95% CI). 33B. Distribution of detected peptide length (x axis) for nuORF (pink) and annotated (grey) peptides (median 9 AA for both). 33C. Ribo-seq translation levels (y axis, log₂(TPM+1)) of annotated proteins (grey) and nuORFs (pink) in B721.221 cells (means: 1.6 annotated, 1.7 nuORF, 5.8% to 11.7% increase, 95% CI). 33D. Predicted hydrophobicity index (y axis) and retention time (x axis) of annotated (grey) and nuORF (pink) peptides for the HLA-B*56:01 sample. Dashed line: Lowess fit to the annotated peptides. 33E. Similar sequence motifs in nuORFs and annotated peptides. NMDS plot of all 9 AA peptides (dots) identified in HLA-B56:01 from nuORF (red) or annotated ORFs (grey). Sequence motif plots shown for all annotated, all nuORF, and two marked clusters. 33F. Entropy weighted correlation (y axis) across all B721 HLA alleles between identified 9 AA annotated peptides and either down-sampled sets of annotated peptides, or nuORF peptides. 33G. Distribution of predicted MHC I binding scores for annotated (black), nuORF (red) and proteasomal spliced (blue) peptides 33H. nuORFs contributing peptides to the MHC I immunopeptidome are shorter than corresponding annotated proteins. Distribution of length (x axis) of different nuORF classes and annotated proteins (y axis) contributing peptides to the MHC I immunopeptidome. 33I. A 5′ uORF from ARAF detected in the MHC I immunopeptidome. Red box: magnified view of the 5′ uORF read coverage. Blue bars: in-frame reads, grey bars: out-of-frame reads. Magenta outline: LC-MS/MS detected peptide with periodicity plot showing strong read support for translation. 33J-33M. nuORFs in the immunopeptidome have distinct characteristics compared to those in the whole proteome. 33J. Percent nuORFs (y axis) in immunopeptidome across 92 HLA alleles (pink) or of the whole proteome (grey). 33K. Number of nuORFs (x axis) of different categories (y axis) detected in the immunopeptidome (left) or the whole proteome (right). 33L. Proportion of all annotated ORFs (top) or nuORFs (bottom) detected in the whole proteome (blue), immunopeptidome (pink) or both (intersection) in B721.221 cells. 33M. Cumulative distribution function plots of Ribo-seq translation levels (left, x axis, log₂(TPM+1)) or protein length (right, x axis) for annotated ORFs (top) or nuORFs (bottom) in MHC I immunopeptidome (red) or the whole proteome (blue). P-values: KS test. A,C,F,H,J: For all boxplots, the median is shown, the 25% and 75% define the box range, and the whiskers go up to 1.5 IQR.

FIG. 34A-34H—nuORF peptides in the MHC I immunopeptidome of cancer cells. 34A-34C. nuORFdb allows detection of nuORFs in MHCI I immunopeptidome of samples and tumors types without prior Ribo-Seq data. 344A. Percent nuORF peptides detected in the MHC I immunopeptidome (y axis) from primary CLL, GBM, melanoma (MEL), ovarian carcinoma (OV), and renal cell carcinoma (RCC) (x axis). Hashed bars: Samples that contributed to nuORFdb. Grey bars: Same cancer types as in nuORFdb but other patients. Black bars/Distinct: Samples not represented in nuORFdb. 34B. Fraction of MS/MS-detected nuORFs (colorbar) in each sample (rows) predicted by each node (columns). 34C. Number of nuORFs (x axis) of different types (y axis) identified in the MHC I immunopeptidome across 12 cancer samples. 34D. More than half of nuORFs are detected in more than one sample. Percent of nuORFs detected in one or more samples, including all cancer samples and B721.221 cells. 34E-34H. Overlap in peptides presented on same HLA alleles. 344E. Approach to analyze peptide overlap between cancer samples and B721.221 cells expressing the same HLA alleles. Dark blue circle: cancer sample with 6 known HLA alleles. Grey circles: HLA mono-allelic B721.221 cells. Blue boxes: B721.221 cells used in the overlap analysis expressing cancer-matched HLA alleles. 34F. Percent of annotated (grey) and nuORF (pink) peptides (y axis) detected in cancer immunopeptidomes (x axis) that are also detected in HLA type-matched B721.221 samples. Number of available B721.221 sampled alleles over cancer sample's known HLA alleles are shown above the bar. 34G. Percent of annotated (black) or nuORF (red) peptides (y axis) detected in cancer MHC I immunopeptidomes that are also detected in 6 B721.221 mono-allelic samples with variable numbers of HLA-matched samples (x axis). 344H. Ribo-seq translation levels (y axis, log₂(TPM+1)) of annotated ORFs (grey) and nuORFs (pink) exclusive to cancer samples or also detected in B721.221 cells (hashed) (t-test, Annotated: p=10⁻¹⁶¹, nuORF: p=10⁻¹³).

FIG. 35A-35L—nuORFs expand the potential neoantigen repertoire in cancer. 35A. Approaches to identify potential nuORF-derived neoantigens. 35B-35E. Potential neoantigens from nuORFs with somatic mutations. 35B. Percent of ORFs with median 30× or higher read coverage (y axis) by WES (n=18 samples: primary cancer MEL and GBM and matched normal (3,4)) and WGS (n=2 samples, hashed) for different types of ORFs (x axis) (*p<0.01, t-test). 35C. Number of Ribo-seq supported, non-synonymous SNVs (y axis) in MEL11 in annotated ORFs, nuORFs, or in both ORF types when they overlap. 35D. Number of high affinity (<500 nM, netMHCpan v4.0) potential neoantigens (y axis) from annotated ORFs (grey) and nuORFs (pink) in MEL11. 35E. The rate of SNV-derived potential neoantigen peptides with high binding affinity (<500 nM, netMHCpan v4.0) (y axis) from annotated ORFs (grey) and nuORFs (pink) across 1,170 netMHCpan v4.0 trained HLA alleles (means: 1.4% annotated, 1.6% nuORFs (0.1-0.3% higher, CI 95%)). For the boxplot, the median is shown, the 25% and 75% define the box range, and the whiskers go up to 1.5 IQR. 35F-35H. MHC I MS/MS-detected nuORFs enriched in cancers may be potential sources of neoantigens. 35F. Expression level (log 2(TPM+1)) of nuORFs (rows) detected in MHC I immunopeptidomes of 6 melanoma samples, ordered by mean expression (rightmost column) across all GTEx tissues (columns), except testis. Red box: nuORF at bottom 10% by mean expression (left), filtered for those expressed at least 2-fold higher in at least 5% of 473 melanoma samples in (TCGA) (right). 35G. Expression level (y axis, log₂(TPM+1)) of melanoma-enriched, MS/MS-detected nuORFs in GTEx (purple) and TCGA melanoma (green) samples (x axis). Blue line: 2× highest GTEx expression (testis excluded). 355H. Percent of TCGA melanoma samples (y axis) with nuORF transcript (x axis) expression greater than 2× highest GTEx expression. 35I-35L. nuORFs specifically translated in cancers as potential sources of neoantigens. 35I. Left: Ribo-seq translation levels (log₂(TPM+1)) of nuORFs (rows) exclusively translated in GBM (pink box), melanoma (green box) or CLL (teal box) samples (columns, left), with median expression <1 TPM across GTEx tissues (columns, middle) (testis excluded), and their expression (log₂(TPM+1)) in respective TCGA tumors (columns, right). Far right: Significantly higher expression (grey, p<0.0001, rank-sum test) in expected cancer type vs. the other cancer types (vs. TCGA) or vs. GTEx expression. 355J. Percent of nuORFs (y axis) for each cancer type (x axis) with significantly higher expression (p<0.0001, rank-sum test) in the expected cancer type than the other two cancer types (grey) or GTEx (purple) samples. 35K. Expression (y axis, log₂(TPM+1)) of CLL-specific nuORFs (x axis) in CLL (teal), GBM (pink), melanoma (green), and GTEx (purple). 35L. CLL-specific ARHGAP44 5′uORF (red box). Alternative transcript isoforms are translated in melanoma vs. CLL, and not translated in B cells. E,G,K: For all boxplots, the median is shown, the 25% and 75% define the box range, and the whiskers go up to 1.5 IQR.

FIG. 36A-36F nuORFdb characteristics. 36A. Hierarchical ORF prediction tree with leaves (samples), branches (tissues) and the root (all reads) showing nodes where ORFs were predicted (arrowheads). Asterisks: samples used in nuORFdb construction, but later discovered to be of poor quality and not used in any subsequent analyses. 36B. Chart showing occurrence of all reads, samples and tissues. 36C. Graph showing number of ORFs specific to a prediction node. 36D. Graph showing node contributions to nuORFdb. 36E-36F. NuORFdb size relative to the transcriptome and the annotated proteome. Number of ORFs (y axis, 36E) and unique 9AA peptides (y axis, 36F) in the entire transcriptome, the nuORFdb, or the annotated UCSC proteome (x axis).

FIG. 37A-37G—Additional filtering of MHC I IP, MS/MS-detected nuORF peptides. 37A-37B. Total number of nuORF peptides (y axis) identified pre-filtering and retained post-filtering (hashed) overall (A) and for different nuORF types (x axis, 37B). 37C-37D. False discovery rate (y axis) for annotated (grey) and nuORF (pink) peptides across 92 HLA alleles pre- and post-filtering (hashed) overall (37C) and for different ORF types (x axis, 37D). 37E. Criteria used to filter peptides across ORF types. 37F. Filter cutoffs (vertical red lines) across different peptide spectral match scoring features (x axis) for different ORF types (y axis). For all boxplots, the median is shown, the 25% and 75% define the box range, and the whiskers go up to 1.5 IQR. 37G. Percent of peptides (y axis) retained post-filtering across different ORF categories and overall (x axis).

FIG. 38A-38G—nuORFs peptides in the MHC I immunopeptidome are comparable to those from annotated ORFs. 38A. Different types of nuORFs were detected in the MHC I immunopeptidome. Number of unique proteins (x axis) detected by MHC I IP LC-MS/MS across expanded ORF types (y axis). 38B-38G. Comparable features of nuORFs and annotated peptides. 38B. LC-MS/MS Spectrum Mill identification score (x axis) for annotated and nuORF peptides across ORF types (y axis). 38C. Peptide fragmentation score (x axis) for peptides identified across ORF types (y axis). 38D. Ribo-seq translation levels (x axis, log₂(TPM+1)) of MHC I MS-detected ORFs across various ORF types (y axis). For all boxplots, the median is shown, the 25% and 75% define the box range, and the whiskers go up to 1.5 IQR. 38E. Predicted hydrophobicity index (y axis) against the LC-MS/MS retention time (x axis) for annotated (grey) and nuORF (pink) peptide sequences for three representative HLA alleles. Dashed line: Lowess fit to the annotated peptides. Sample sizes, root mean square errors (rmse), and p-values (rank-sum test on residuals) are marked. 38F-38G. Similar sequence motifs in nuORFs and annotated peptides. 38F. Non-metric multidimensional scaling (NMDS) plot of all MHC IP LC-MS/MS-detected annotated and nuORF 9 AA peptide sequences clustered by peptide sequence similarity for three representative HLA alleles. 38G. Consensus peptide sequence motif plots of all MHC IP LC-MS/MS-detected annotated and nuORF 9 AA peptide sequences.

FIG. 39A-39D—Hierarchical ORF prediction based on Ribo-seq allows to identify short, overlapping, tissue-specific nuORFs. 39A. nuORFs predictions are more sample and tissue specific than annotated ORFs. Proportion of annotated ORFs (grey) and nuORFs (pink) in the MHC I immunopeptidome (y axis, and pie chart). Hatched: proportion predicted only at the leaf and branch level, but not at the root. 39B. Hierarchical ORF prediction approach identifies tissue-specific, overlapping nuORFs. Example of two overlapping, MHC I MS-detected 5′ uORFs in LUZP1. uORF2 (pink) was predicted at the CLL branch, and not at the root. uORF1 (cyan) was predicted at the root and not at the CLL branch. Detected peptides outlined in red with the HLA alleles where peptides were detected marked below. 39C. Ribo-seq allows to identify short ORFs proximal to long annotated ORFs. RNA-seq and Ribo-seq reads aligned to the transcript of the MLEC gene. RNA-seq reads align to the entire length of the transcript, while Ribo-seq reads align exclusively to the translated portions. Ribo-seq supports translation of a 5′ uORF (red box, top). Histogram of reads supporting translation of the MLEC 5′ uORF (dark green) (bottom). 39D. Ribo-seq allows to identify short, overlapping nuORFs. SOCS1 gene encodes three translated proteins: the annotated ORF, a novel out-of-frame iORF, and a 5′ overlap ouORF. Two MHC I MS-detected peptides from 5′ ouORF outlined in yellow. Detected iORF peptide outlined in red and shown in higher magnification below. Bottom: Histogram of Ribo-seq reads supporting translation of the annotated ORF (blue) and the out-of-frame iORF (green).

FIG. 40A-40D—Spectra of proteasomal spliced peptides frequently map to nuORF peptides. 40A-40B. Proposed spliced peptide sequences can be more readily explained by a match to nuORFdb. 40A. LC-MS/MS spectrum of the peptide ALLFWENKL presented by HLA allele A02:04 was previously identified as a cis-spliced peptide which can also be explained by the translated LINC01055 lncRNA nuORF. 40B. RNA-seq and Ribo-seq reads aligned to the LINC01055 lncRNA locus. Red box marks the MHC IIP LC-MS/MS detected nuORF. Bottom panel shows a magnified view of the reads supporting nuORF translation. 40C-40D. Partial sequence present in the MS/MS spectra assigned to spliced peptides are also consistent with different, yet similar, nuORF peptide sequences. 40C. LC-MS/MS spectrum from an allele A31:01 consistent with a cis-spliced peptide LAKAAAFGR as well as a peptide ALKAAAFGR from a translated KDMSC 5′ uORF. Leucine in position 2 is consistent with the anchor motif for allele A31:01. In addition, 3 more peptides from KDMSC 5′ uORF were detected on HLA A74:01 and C16:01, further supporting the nuORF translation and presentation 40D. RNA-seq and Ribo-seq reads aligned to the KDMSC locus. Red box marks the 5′ uORF detected by MHC I IP LC-MS/MS. Detected peptides and the expected peptide sequence motifs are outlined in yellow and orange.

FIG. 41A-41B—Short nuORFs are presented on MHC I without post-translational protease processing. 41A. The sequence of the 5′ uORF from the ARAF gene and the expected HLA motif for the allele where it was detected. 41B. LC-MS/MS spectrum of the ARAF 5′ uORF.

FIG. 42A-4211—nuORF peptides in the MHC I immunopeptidome and whole proteome of cancer cells. 42A. Total number of MHC I LC-MS/MS spectra mapped (y axis) across cancer samples (x axis). 42B-42D. nuORFs of various types were detected in the MHC I immunopeptidome of cancer samples. Number (42B) and proportion (42C) of nuORFs (y axis) of different types identified in each cancer sample (x axis). 42D. Fraction (y axis) of nuORF types (x axis) in B721.221 cells (dark grey) or across cancer samples (light grey). Asterisk: p<0.05, rank-sum test. For boxplot, the median is shown, the 25% and 75% define the box range, and the whiskers go up to 1.5 IQR. 42E-42H. nuORFs are more abundant in the MHC I immunopeptidome than in the whole proteome. 42E. Percent of nuORF peptides (y axis) detected in the immunopeptidome (pink) and in the whole proteome (blue) of GBM6. 42F. Number of nuORFs (x axis) of different types (y axis) identified in the MHC I immunopeptidome vs. whole proteome (hatched) in GBM6. 42G. Protein length (x axis, amino acids) of annotated (top) and nuORF (bottom) proteins detected in the MHC I immunopeptidome (pink) vs. in the whole proteome (blue). p-values: KS test. 42H. Proportion of all annotated ORFs (top) or nuORFs (bottom) detected in the whole proteome (blue), immunopeptidome (pink) or both (intersection) in GBM6.

FIG. 43A-43E—nuORFs can be potential sources of neoantigens. 43A. Approaches to identify potential nuORF-derived neoantigens. 43B. nuORFs have low sequence coverage by WES as compared to WGS. WES read coverage (x axis) across different ORF types (y axis). Bottom: WGS read coverage across all ORFs of all types. For boxplot, the median is shown, the 25% and 75% define the box range, and the whiskers go up to 1.5 IQR. 43C. Somatic variants in the melanoma patient-derived cell line reflect the variants detected in the original tumor. Cancer-specific SNVs and InDels identified by WES from the primary tumor and by WGS from the tumor-derived cell line. 43D. Ribo-seq can be used to identify translated variants. Example of a translated SLC7A1 5′ uORF with a cancer-specific SNV. Top: histogram of Ribo-seq reads supporting the translation of the 5′ uORF. Middle: Ribo-seq reads supporting translation of the mutant (green) and wild-type alleles. Predicted neoantigen outlined in red. 43E. Ribo-seq can be used to select neoantigens. Among 20 variants selected for the neoantigen vaccine in MEL11, 2 were shown to be immunogenic. 2/2 immunogenic variants and 5/18 non-immunogenic variants had Ribo-seq support.

FIG. 44A-44C—GBM and MEL specific nuORFs. 44A-44B. GBM-specific nuORFs. 44A. RNA-seq expression (y axis, log₂(TPM+1)) of GBM-specific nuORFs (x axis) in GTEx, TCGA samples (MEL, GBM) and CLL. 44B. LC-MS/MS spectrum of a peptide from SOX2-OT nuORF. 44C. MEL-specific nuORFs. RNA-seq expression (y axis, log₂(TPM+1)) of MEL-specific nuORFs (x axis) in GTEx, TCGA samples (MEL, GBM) and CLL. For all boxplots, the median is shown, the 25% and 75% define the box range, and the whiskers go up to 1.5 IQR.

FIG. 45. Shows the many types of translated unannotated ORFs that have been identified by Ribo-seq.

FIG. 46. Schematic illustrating the potential for undiscovered neoantigens in nuORFs.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to US20190060428A1 which is the U.S. National Phase Application of International Patent Application No. PCT/US2016/036605; US20180000913A1 which is the U.S. National Phase Application of International Patent Application No. PCT/US2015/067154; US20160101170A1 which is the U.S. National Phase Application of International Patent Application No. PCT/US2014/033185; US20160339090A1 which is the U.S. National Phase Application of International Patent Application No. PCT/US2014/071707; US20180153975A1 which is the U.S. National Phase Application of International Patent Application No. PCT/US2016/033452; and U.S. patent Ser. No. 10/426,824B1. All publications, published patent documents, and patent applications cited herein are hereby incorporated by reference to the same extent as though each individual publication, published patent document, or patent application was specifically and individually indicated as being incorporated by reference.

### OVERVIEW

Embodiments disclosed herein provide a method of identifying peptides, e.g., neoantigens, including, but not limited to novel unannotated open reading frames (nuORFs), that are capable of eliciting a cancer specific T-cell response. The enhanced Ribo-seq method described herein can be used to identify novel unannotated open reading frames (nuORFs), which are an untapped source of neoantigens for cancer immunotherapy. In certain embodiments, the combined identification of neoantigens from annotated protein-coding ORFs and nuORFs can be used to generate improved immunotherapies, such as a vaccine comprising the identified peptides or T cells that specifically target the identified peptides (e.g., T cells expressing an endogenous T cell receptor or CART cells). Embodiments disclosed herein also provide for methods of priming, activating and expanding neoantigen-targeting T cells. Embodiments disclosed herein also provide for personalized and shared immunogenic compositions (e.g., vaccines or T cells).

Methods for identification of neoantigens is provided, which may comprise the steps of performing Ribosomal profiling (Ribo-seq) on a sample or set of samples; generating a novel untranslated open reading frame (nuORF) database comprising predicted nuORFs by conducting hierarchical ORF prediction on the Ribo-seq data generated; and generating a final set of neoantigens by searching the nuORF database for predicted nuORFs in the nuORF database matching data in a MHC I immunopeptidome data set, the identified predicted nuORFs comprising the final neoantigen set. Such a set can be provided in a library for further use/

Methods for preparing neopolypeptides and neoantigens are also provided. In one aspect, the invention provides a method for preparing a neoantigen for an immunogenic pharmaceutical composition, wherein the neoantigen is specific to a subject that has a cancer, wherein the neoantigen is specific to the subject's cancer, wherein the neoantigen binds to an HLA protein of the subject, and wherein the neoantigen comprises a subject-specific amino acid sequence expressed by cancer cells of the subject but not expressed by non-cancer cells of the subject that is encodes a mutated coding sequence of the subject's cancer cells (neo-ORF) or nuORF. In embodiments, the method for preparing a neoantigen comprises comparing cancer and non-cancer cellular translation products of the subject comprising: (a) extracting from cancer cells of the subject a sample of ribosomes containing ribosome-protected mRNA fragments (RPFs), (b) removing rRNA from the RPFs to obtain rRNA-removed RPFs, (c) purifying the rRNA-removed RPFs to obtain purified RPFs, (d) preparing a library of purified circular DNA (cDNA) from the purified RPFs, said purified cDNA having open reading frames (ORFs), and (e) identifying the neo-ORF of the purified cDNA that encodes the neoantigen from cancer cells by comparing ORFs of purified cDNA with ORFs of non-cancer cells.

One or more neopolypeptides or one or more neoantigens can be provided in a library or a pharmaceutical composition, and methods of treatment using the neopolypeptides and neoantigens of the present invention are also provided.

### Neoantigens and Neopolypeptides

The present invention is based, at least in part, on the ability to present the immune system of the patient with a pool of disease or disorder-specific neoantigens or neopolypeptides. As used herein, the term “neoantigen” or “neoantigenic” means (1) a class of tumor antigens that arises from a tumor-specific mutation(s) which alters the amino acid sequence of genome encoded proteins; (2) a class of tumor antigens having tumor specific expression that arises from retained introns, alternative open reading frames (ORFs) within coding genes, antisense transcripts, defective ribosomal products (DRiPs), “non-coding” regions of the genome, 5′ and 3′ untranslated regions (UTRs), overlapping yet out-of-frame alternative ORFs in annotated protein-coding genes, long non-coding RNAs (lncRNAs), pseudogenes and other transcripts currently annotated as non-protein coding; or (3) novel unannotated open reading frames (nuORFs) that arise from a tumor-specific mutation(s) in unannotated open reading frames. The neoantigens, or neoepitopes, or neopolypeptides may also be subject specific. Neoantigen compositions comprising one or more neoantigens are disclosed herein. In particular embodiments, the compositions may comprise cancer specific neoantigens, pathogen specific neopolypeptides, or genetic disorder specific neopolypeptides. One of skill in the art from this disclosure and the knowledge in the art will appreciate that there are a variety of ways in which to produce such specific neoantigens. In general, such neoantigens may be produced either in vitro or in vivo. Specific neoantigens may be produced in vitro as peptides or polypeptides, which may then be formulated into a neoplasia vaccine or immunogenic pharmaceutical composition and administered to a subject. As described in further detail herein, such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, cancer specific neoantigens may be produced in vivo by introducing molecules (e.g., DNA, RNA, viral expression systems, and the like) that encode tumor specific neoantigens into a subject, whereupon the encoded tumor specific neoantigens are expressed. The methods of in vitro and in vivo production of neoantigens is also further described herein as it relates to pharmaceutical compositions and methods of delivery of the therapy. In certain embodiments, neoantigen formulations are prepared as in US20190060428A1, which is the U.S. National Phase Application of International Patent Application No. PCT/US2016/036605.

Polypeptides comprising one or more neoantigens are provided herein. In embodiments, the polypeptide comprises 2 or more neoantigenes, which can be linked together directly, or with any of the linkers disclosed herein. In particular embodiments, the polypeptide comprises a T cell enhancer amino acid sequence.

T cell enhancer amino acids may be selected from the group consisting of an invariant chain; a leader sequence of tissue-type plasminogen activator; a PEST sequence, a cyclin destruction box; a ubiquitination signal; and a SUMOylation signal. See, e.g. Nguyen et al., Front. Immunol. 2015, doi:10.3389/fimmu.2015.00462. Neoantigen compositions are provided herein, and may comprise one or more neoantigens from Table 1-3D, e.g. Table 1, 2A, 2B, 3A, 3B, 3C, 3D. A polynucleotide encoding the polypeptides disclosed herein may also be provided. The compositions may comprise 2 or more, 3, or more, 4 or more up to 20 or more neoantigens, or at least one polynucleotide that encodes the one or more neoantigens. The composition may further comprise one or more adjuvants. The composition may be provided on one or more vectors as disclosed herein. The vector in particular embodiments may comprise a self-amplifying RNA vector or an adenovirus vector.

In some embodiments, the subject's cancer is a solid tumor, hematological cancer, breast cancer, ovarian cancer, prostate cancer, lung cancer, kidney cancer, gastric cancer, colon cancer, testicular cancer, head and neck cancer, pancreatic cancer, brain cancer, bladder cancer, melanoma, lymphoma or leukemia.

In certain embodiments, the present invention includes modified neoantigenic peptides. As used herein in reference to neoantigenic peptides, the terms “modified”, “modification” and the like refer to one or more changes that enhance a desired property of the neoantigenic peptide, where the change does not alter the primary amino acid sequence of the neoantigenic peptide. “Modification” includes a covalent chemical modification that does not alter the primary amino acid sequence of the neoantigenic peptide itself. Such desired properties include, for example, prolonging the in vivo half-life, increasing the stability, reducing the clearance, altering the immunogenicity or allergenicity, enabling the raising of particular antibodies, cellular targeting, antigen uptake, antigen processing, MHC affinity, MHC stability, or antigen presentation. Changes to a neoantigenic peptide that may be carried out include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naïve or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines. The human leukocyte antigen (HLA) system is a gene complex encoding the major histocompatibility complex (MHC) proteins in humans.

By “proteins or molecules of the major histocompatibility complex (MHC)”, “WIC molecules”, “MHC proteins” or “HLA proteins” is thus meant proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. Tumor antigen-specific T-cells can be developed utilizing the immunogenic compositions as disclosed herein. Neopeptides capable of associating with different MHC molecules, such as different MHC class I molecules and/or different MHC class II molecules are envisioned for use as described herein. In some aspects, immunogenic compositions comprise neopeptides and/or sequences encoding the peptide that are capable of associating with the MHC class I molecules and/or MHC class II molecules. The immunogenic compositions can comprise different fragments capable of associating with 2 or more or 3 or MHC class I molecules and/or class II molecules. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens. Accordingly, immunogenic compositions can be made according to the present invention with the neoantigenic peptides as disclosed herein, capable of raising a specific cytotoxic T-cells response and/or a specific helper T-cell response.

MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin.

MHC molecules of class II consist of an α-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

Subject specific HLA alleles or HLA genotype of a subject may be determined by any method known in the art. In preferred embodiments, HLA genotypes are determined by any method described in International Patent Application number PCT/US2014/068746, published Jun. 11, 2015 as WO2015085147. Briefly, the methods include determining polymorphic gene types that may comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

The clinical effectiveness of protein therapeutics is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, but certain embodiments have a molecular weight between 500 and 20,000 while other embodiments have a molecular weight between 4,000 and 10,000.

The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to a polypeptide of the present disclosure via a terminal reactive group (a “spacer”). The spacer is, for example, a terminal reactive group which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxy succinylimide. Another activated polyethylene glycol which may be bound to a free amino group is 2,4-bis(0-methoxypolyethyleneglycol)-6-chloro-s-triazine which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C. The present disclosure also contemplates the use of PEG Mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present. Glycosylation can dramatically affect the physical properties of proteins and can also be important in protein stability, secretion, and subcellular localization. Proper glycosylation can be essential for biological activity. In fact, some genes from eucaryotic organisms, when expressed in bacteria (e.g., E. coli) which lack cellular processes for glycosylating proteins, yield proteins that are recovered with little or no activity by virtue of their lack of glycosylation.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the DNA level, particularly by mutating the DNA encoding the polypeptide at preselected bases such that codons are generated that will translate into the desired amino acids. Another means of increasing the number of carbohydrate moieties on the polypeptide is by chemical or enzymatic coupling of glycosides to the polypeptide.

Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Dihydrofolate reductase (DHFR)—deficient Chinese Hamster Ovary (CHO) cells are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2): 125-30). As with modifications with other conjugates (e.g., PEG), various techniques for site-specific polysialylation are available (see, e.g., T. Lindhout et al., PNAS 108(18)7397-7402 (2011)).

Additional suitable components and molecules for conjugation include, for example, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more polypeptides of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake, incorporation and expression of exogenous genetic material (exogenous DNA) from its surroundings and taken up through the cell membrane(s). Transformation occurs naturally in some species of bacteria, but it can also be effected by artificial means in other cells.

Furthermore, albumin itself may be modified to extend its circulating half-life. Fusion of the modified albumin to one or more Polypeptides can be attained by the genetic manipulation techniques described above or by chemical conjugation; the resulting fusion molecule has a half-life that exceeds that of fusions with non-modified albumin. (See WO2011/051489).

Several albumin—binding strategies have been developed as alternatives for direct fusion, including albumin binding through a conjugated fatty acid chain (acylation). Because serum albumin is a transport protein for fatty acids, these natural ligands with albumin—binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule.

A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein (e.g., diabetes).

A polypeptide may also be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

A “receptor” is to be understood as meaning a biological molecule or a molecule grouping capable of binding a ligand. A receptor may serve, to transmit information in a cell, a cell formation or an organism. The receptor comprises at least one receptor unit and frequently contains two or more receptor units, where each receptor unit may consist of a protein molecule, in particular a glycoprotein molecule. The receptor has a structure that complements the structure of a ligand and may complex the ligand as a binding partner. Signaling information may be transmitted by conformational changes of the receptor following binding with the ligand on the surface of a cell. According to the invention, a receptor may refer to particular proteins of MHC classes I and II capable of forming a receptor/ligand complex with a ligand, in particular a peptide or peptide fragment of suitable length.

Purification methods such as cation exchange chromatography may be used to separate conjugates by charge difference, which effectively separates conjugates into their various molecular weights. For example, the cation exchange column can be loaded and then washed with −20 mM sodium acetate, pH −4, and then eluted with a linear (0 M to 0.5 M) NaCl gradient buffered at a pH from about 3 to 5.5, e.g., at pH −4.5. The content of the fractions obtained by cation exchange chromatography may be identified by molecular weight using conventional methods, for example, mass spectroscopy, SDS-PAGE, or other known methods for separating molecular entities by molecular weight.

In certain embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration.

Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

The present disclosure contemplates the use of other modifications, currently known or developed in the future, of the polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

### In Vitro Peptide/Polypeptide Synthesis

Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963). In certain embodiments, neoantigenic peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Alternatively, a nucleic acid (e.g., a polynucleotide) encoding a neoantigenic peptide of the invention may be used to produce the neoantigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In one embodiment in vitro translation is used to produce the peptide. Many exemplary systems exist that one skilled in the art could utilize (e.g., Retic Lysate IVT Kit, Life Technologies, Waltham, Mass.).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The neoantigenic peptides may be provided in the form of RNA or cDNA molecules encoding the desired neoantigenic peptides. One or more neoantigenic peptides of the invention may be encoded by a single expression vector.

A vector system comprising one or more expression vectors are disclosed herein, including wherein each expression vector is selected from the group consisting of a plasmid, a cosmid, a RNA, a RNA formulated in a particle, a self-amplifying RNA (SAM), a SAM formulated in a particle, or a viral vector. Viral vectors can in some embodiments be an alpha virus vector, a Venezuelan equine encephalitis (VEE) virus vector, a sindbis virus vector, a semliki forest virus vector, a simian or human cytomegalovirus vector, a lymphocyte choriomenigitis virus vector, a retroviral vector, a lentiviral vector, an adenovirus vector, or combination thereof. See, e.g. Naslund et al., Virology Journal 8, 36 (2011); Knudsen et al., doi:10.1128/JVI.02223-14.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific neoantigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the tumor specific neoantigenic peptide fused in the same reading frame to a marker sequence that allows, for example, for purification of the encoded polypeptide, which may then be incorporated into the personalized neoplasia vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific neoantigenic peptides fused in the same reading frame to create a single concatamerized neoantigenic peptide construct capable of producing multiple neoantigenic peptides.

In certain embodiments, isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neoantigenic peptide of the present invention, can be provided.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The isolated tumor specific neoantigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding the tumor specific neoantigenic peptides. Recombinant expression vectors are replicable DNA constructs which have synthetic or cDNA-derived DNA fragments encoding a tumor specific neoantigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

### In Vivo Peptide/Polypeptide Synthesis

The present invention also contemplates the use of nucleic acid molecules as vehicles for delivering neoantigens to the subject in need thereof, in vivo, in the form of, e.g., DNA/RNA vaccines (see, e.g., WO2012/159643, and WO2012/159754, hereby incorporated by reference in their entirety).

In one embodiment, neoantigens may be administered to a patient in need thereof by use of a plasmid. These are plasmids which usually consist of a strong viral promoter to drive the in vivo transcription and translation of the gene (or complementary DNA) of interest (Mor, et al., (1995). The Journal of Immunology 155 (4): 2039-2046). Intron A may sometimes be included to improve mRNA stability and hence increase protein expression (Leitner et al. (1997). The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999). Adv. Parasitol. Advances in Parasitology 42: 343-410; Robinson et al., (2000). Adv. Virus Res. Advances in Virus Research 55: 1-74; Bohmet al., (1996). Journal of Immunological Methods 193 (1): 29-40.). Multicistronic vectors are sometimes constructed to express more than one immunogen, or to express an immunogen and an immunostimulatory protein (Lewis et al., (1999). Advances in Virus Research (Academic Press) 54: 129-88).

Because the plasmid is the “vehicle” from which the immunogen is expressed, optimizing vector design for maximal protein expression is essential (Lewis et al., (1999). Advances in Virus Research (Academic Press) 54: 129-88). One way of enhancing protein expression is by optimizing the codon usage of pathogenic mRNAs for eukaryotic cells. Another consideration is the choice of promoter. Such promoters may be the SV40 promoter or Rous Sarcoma Virus (RSV).

Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermally (ID), with DNA being delivered to the extracellular spaces. This can be assisted by electroporation by temporarily damaging muscle fibres with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex Vivo Cytosolic Delivery of Functional Macromolecules to Immune Cells, Sharei et al, PLOS ONE DOI:10.1371/journal.pone.0118803 Apr. 13, 2015).

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshiaet al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic pharmaceutical composition may include separate DNA plasmids encoding, for example, one or more neoantigens as identified in according to the invention. As discussed herein, the exact choice of expression vectors can depend upon the neoantigens to be expressed, and is well within the skill of the ordinary artisan. The expected persistence of the DNA constructs (e.g., in an episomal, non-replicating, non-integrated form in the muscle cells) is expected to provide an increased duration of protection.

One or more neoantigens of the invention may be encoded and expressed in vivo using a viral based system (e.g., an adenovirus system, an adeno associated virus (AAV) vector, a poxvirus, or a lentivirus). In one embodiment, the neoplasia vaccine or immunogenic pharmaceutical composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference).

The neoantigens of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the neoantigens of the invention. Upon introduction into an acutely or chronically infected host or into a noninfected host, the vector expresses the immunogenic neoantigens, and thereby elicits a host CTL response.

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g., US Patent Publication Nos. US20110293571; US20040013648, US20070025970, US20090111106 and U.S. Pat. No. 7,259,015. In another embodiment lentiviral vectors are used to deliver vectors to the brain of those being treated for a disease.

As to lentivirus vector systems useful in the practice of the invention, mention is made of U.S. Pat. Nos. 6,428,953, 6,165,782, 6,013,516, 5,994,136, 6,312,682, and 7,198,784, and documents cited therein.

In an embodiment herein the delivery is via an lentivirus. Zou et al. administered about 10₁1.1 of a recombinant lentivirus having a titer of 1×10⁹ transducing units (TU)/ml by an intrathecal catheter. These sort of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 10⁸ TU/ml, preferably from 10⁸ to 10⁹ TU/ml, more preferably at least 10⁹ TU/ml. Other methods of concentration such as ultrafiltration or binding to and elution from a matrix may be used.

In other embodiments the amount of lentivirus administered may be 1×10⁵ or about 1×10⁵ plaque forming units (PFU), 5×10⁵ or about 5×10⁵ PFU, 1×10⁶ or about 1×10⁶ PFU, 5×10⁶ or about 5×10⁶ PFU, 1×10⁷ or about 1×10⁷ PFU, 5×10⁷ or about 5×10⁷ PFU, 1×10⁸ or about 1×10⁸ PFU, 5×10⁸ or about 5×10⁸ PFU, 1×10⁹ or about 1×10⁹ PFU, 5×10⁹ or about 5×10⁹ PFU, 1×10¹⁰ or about 1×10¹⁰ PFU or 5×10¹⁰ or about 5×10¹⁰ PFU as total single dosage for an average human of 75 kg or adjusted for the weight and size and species of the subject. One of skill in the art can determine suitable dosage. Suitable dosages for a virus can be determined empirically.

Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the invention, mention is made of U.S. Pat. No. 6,955,808. The adenovirus vector used can be selected from the group consisting of the Ad5, Ad35, Ad11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates WIC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which all viral genes are deleted, can also be used in accordance with the present invention. Such vectors require a helper virus for their replication and require a special human 293 cell line expressing both Ela and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention, and can even be used for co-delivery of a large number of heterologous inserts/genes.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10′ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10′ pu, about 2×10′ pu, about 4×10′ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: SynapsinI for all neurons, CaMKIIalpha for excitatory neurons, GAD67 or GAD65 or VGAT for GABAergic neurons, etc. Promoters used to drive RNA synthesis can include: Pol III promoters such as U6 or H1. The use of a Pol II promoter and intronic cassettes can be used to express guide RNA (gRNA).

With regard to AAV vectors useful in the practice of the invention, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment effectively activating a cellular immune response for a neoplasia vaccine or immunogenic composition can be achieved by expressing the relevant neoantigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety).

In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (see e.g., Verardiet al., Hum Vaccin Immunother. 2012 Jul.; 8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

Information concerning poxviruses that may be used in the practice of the invention, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC.1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature, such as:

U.S. Pat. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756,101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and

Panicali, D. Proc. Natl. Acad. Sci. 1982; 79; 4927-493, Panicali D. Proc. Natl. Acad. Sci. 1983; 80(17): 5364-8, Mackett, M. Proc. Natl. Acad. Sci. 1982; 79: 7415-7419, Smith G L. Proc. Natl. Acad. Sci. 1983; 80(23): 7155-9, Smith G L. Nature 1983; 302: 490-5, Sullivan V J. Gen. Vir. 1987; 68: 2587-98, Perkus M Journal of Leukocyte Biology 1995; 58:1-13, Yilma T D. Vaccine 1989; 7: 484-485, Brochier B. Nature 1991; 354: 520-22, Wiktor, T J. Proc. Natl Acd. Sci. 1984; 81: 7194-8, Rupprecht, C E. Proc. Natl Acd. Sci. 1986; 83: 7947-50, Poulet, H Vaccine 2007; 25(Jul.): 5606-12, Weyer J. Vaccine 2009; 27(Nov.): 7198-201, Buller, R M Nature 1985; 317(6040): 813-5, Buller R M. J. Virol. 1988; 62(3):866-74, Flexner, C. Nature 1987; 330(6145): 259-62, Shida, H. J. Virol. 1988; 62(12): 4474-80, Kotwal, G J. J. Virol. 1989; 63(2): 600-6, Child, S J. Virology 1990; 174(2): 625-9, Mayr A. Zentralbl Bakteriol 1978; 167(5,6): 375-9, Antoine G. Virology. 1998; 244(2): 365-96, Wyatt, L S. Virology 1998; 251(2): 334-42, Sancho, M C. J. Virol. 2002; 76(16); 8313-34, Gallego-Gomez, J C. J. Virol. 2003; 77(19); 10606-22), Goebel S J. Virology 1990; (a,b) 179: 247-66, Tartaglia, J. Virol. 1992; 188(1): 217-32, Najera J L. J. Virol. 2006; 80(12): 6033-47, Najera, J L. J. Virol. 2006; 80: 6033-6047, Gomez, C E. J. Gen. Virol. 2007; 88: 2473-78, Mooij, P. Jour. Of Virol. 2008; 82: 2975-2988, Gomez, C E. Curr. Gene Ther. 2011; 11: 189-217, Cox, W. Virology 1993; 195: 845-50, Perkus, M. Jour. Of Leukocyte Biology 1995; 58: 1-13, Blanchard T J. J Gen Virology 1998; 79(5): 1159-67, Amara R. Science 2001; 292: 69-74, Hel, Z., J. Immunol. 2001; 167: 7180-9, Gherardi M M. J. Virol. 2003; 77: 7048-57, Didierlaurent, A. Vaccine 2004; 22: 3395-3403, Bissht H. Proc. Nat. Aca. Sci. 2004; 101: 6641-46, McCurdy L H. Clin. Inf. Dis 2004; 38: 1749-53, Earl P L. Nature 2004; 428: 182-85, Chen Z. J. Virol. 2005; 79: 2678-2688, Najera J L. J. Virol. 2006; 80(12): 6033-47, Nam J H. Acta. Virol. 2007; 51: 125-30, Antonis A F. Vaccine 2007; 25: 4818-4827, B Weyer J. Vaccine 2007; 25: 4213-22, Ferrier-Rembert A. Vaccine 2008; 26(14): 1794-804, Corbett M. Proc. Natl. Acad. Sci. 2008; 105(6): 2046-51, Kaufman H L., J. Clin. Oncol. 2004; 22: 2122-32, Amato, R J. Clin. Cancer Res. 2008; 14(22): 7504-10, Dreicer R. Invest New Drugs 2009; 27(4): 379-86, Kantoff P W. J. Clin. Oncol. 2010, 28, 1099-1105, Amato R J. J. Clin. Can. Res. 2010; 16(22): 5539-47, Kim, D W. Hum. Vaccine. 2010; 6: 784-791, Oudard, S. Cancer Immunol. Immunother. 2011; 60: 261-71, Wyatt, L S. Aids Res. Hum. Retroviruses. 2004; 20: 645-53, Gomez, C E. Virus Research 2004; 105: 11-22, Webster, D P. Proc. Natl. Acad. Sci. 2005; 102: 4836-4, Huang, X. Vaccine 2007; 25: 8874-84, Gomez, C E. Vaccine 2007a; 25: 2863-85, Esteban M. Hum. Vaccine 2009; 5: 867-871, Gomez, C E. Curr. Gene therapy 2008; 8(2): 97-120, Whelan, K T. Plos one 2009; 4(6): 5934, Scriba, T J. Eur. Jour. Immuno. 2010; 40(1): 279-90, Corbett, M. Proc. Natl. Acad. Sci. 2008; 105: 2046-2051, Midgley, C M. J. Gen. Virol. 2008; 89: 2992-97, Von Krempelhuber, A. Vaccine 2010; 28: 1209-16, Perreau, M. J. Of Virol. 2011; October: 9854-62, Pantaleo, G. Curr Opin HIV-AIDS. 2010; 5: 391-396, each of which is incorporated herein by reference.

In another embodiment the vaccinia virus is used in the neoplasia vaccine or immunogenic composition to express a neoantigen. (Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In another embodiment ALVAC is used as a vector in a neoplasia vaccine or immunogenic composition. ALVAC is a canarypox virus that can be modified to express foreign transgenes and has been used as a method for vaccination against both prokaryotic and eukaryotic antigens (Honig H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000; 6:2219-28; Musey L, Ding Y, Elizaga M, et al. HIV-1 vaccination administered intramuscularly can induce both systemic and mucosal T cell immunity in HIV-1-uninfected individuals. J Immunol 2003; 171:1094-101; Paoletti E. Applications of pox virus vectors to vaccination: an update. Proc Natl Acad Sci USA 1996; 93:11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall J L, Hawkins M J, Tsang K Y, et al. Phase I study in cancer patients of a replication-defective avipox recombinant vaccine that expresses human carcinoembryonic antigen. J Clin Oncol 1999; 17:332-7).

In another embodiment a Modified Vaccinia Ankara (MVA) virus may be used as a viral vector for a neoantigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA virus contains 31 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. January 2012; 61(1): 19-29). Methods to make and use recombinant MVA has been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment recombinant viral particles of the vaccine or immunogenic composition are administered to patients in need thereof. Dosages of expressed neoantigen can range from a few to a few hundred micrograms, e.g., 5 to 500. mu.g. The vaccine or immunogenic composition can be administered in any suitable amount to achieve expression at these dosage levels. The viral particles can be administered to a patient in need thereof or transfected into cells in an amount of about at least 10³⁵ pfu; thus, the viral particles are preferably administered to a patient in need thereof or infected or transfected into cells in at least about 10⁴ pfu to about 10⁶ pfu; however, a patient in need thereof can be administered at least about 10⁸ pfu such that a more preferred amount for administration can be at least about 10′ pfu to about 10⁹ pfu. Doses as to NYVAC are applicable as to ALVAC, MVA, MVA-BN, and avipoxes, such as canarypox and fowlpox.

### Enhanced Ribo-Seq

Conventional Ribosome profiling (Ribo-seq) allows identification of translated open reading frames (ORFs) by sequencing mRNA fragments protected by the ribosome. Unlike RNA sequencing (RNA-seq), which only shows what is being transcribed, Ribo-seq allows identification of RNA that is being translated. (See Ingolia N T, Ribosome profiling: new views of translation, from single codons to genome scale, Nature Reviews. Genetics, 15 (3): 205-13). Ribo-seq have been used to identify a range of short and non-ATG-initiated ORFs that can generate stable and spatially distinct proteins. (See, Jackson et al., The translation of non-canonical open reading frames controls mucosal immunity, Nature, published Dec. 12, 2018, doi.org/10.1038/s41586-018-0794-7).

Described herein is an enhanced Ribo-seq method that can be used to predict translated unannotated ORFs. Several novel unannotated ORFs (nuORFs) have been identified, e.g., 5′ extension ORFs, 5′ truncation ORFs, within ORFs, overlap 5′ uORFs, 5′ uORFs, overlap 3′ dORFs, 3′ dORFs, and noncoding ORFs.

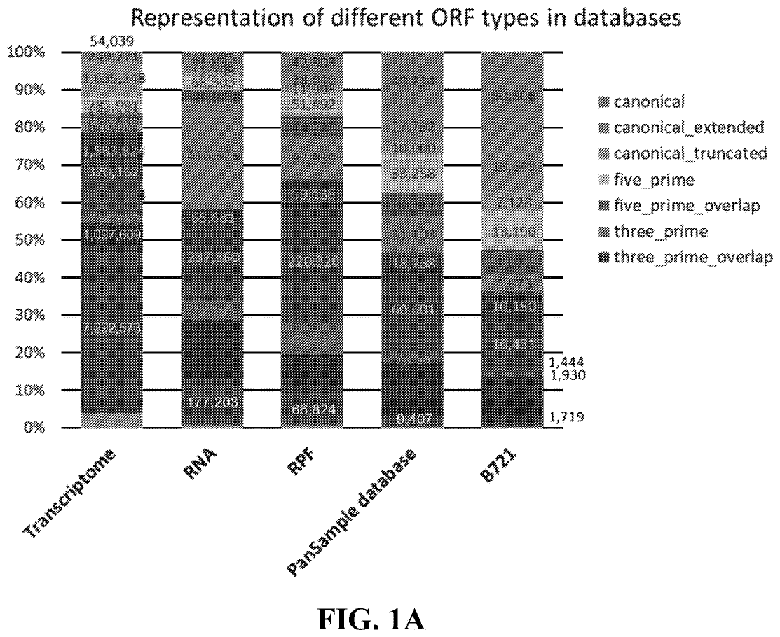

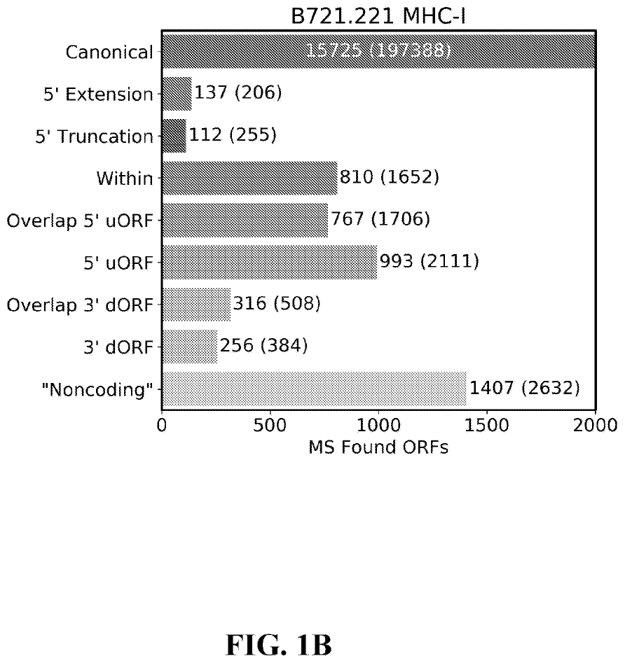

In one aspect, the invention provides a collection of translated neoantigens or neopolypeptides obtained by the enhanced Ribo-Seq method described herein. Methods of identifying netoantigens may comprise the steps of performing Ribo-seq on a sample or set of samples, generating a novel intranslated open reading frame (nuORF) database comprising predicted nuORFs by conducted hierarchical ORF prediction on the Ribo-seq data generated; and generating a final set of neoantigens by searching the nuORF database for predicted nuORFS in the nuPRG database matching data in a MHC 1 immunopeptidome data set, the identified predicted nuORFs comprising the final neoantigen set. Subsequent to performing ribosomial profiling (ribo-seq) on a sample or a set of samples, in methods for identifying neoantigens, a novel untrainslated open reading frame databse can be generated by conducting hierarchical ORF prediction on the Ribo-seq data generated from ribosomal profiling methods. Conducting hierarchical ORF prediction on generated Ribo-seq data can advantageously be used to maximize detection of translated ORF and/or overcome noise from overlapping ORFs expressed in different tissues. In embodiments, hierarchical ORF predictions can be performed using bioinformatic methods to analyze ribosome profiling data. Ribosome profiling can be utilized to determine which RNAs are translated by using computational methods such as Support Vector Machine classifiers to analyze data from ribosome profiling. In embodiments, the methods can be as described by Ji et al, Elife 4(2015). Particular start and stop codons can be designated for identification, in an aspect, ORFs with NTG start codons and TAA/TGA/TAG stop codons can be identified. RibORF and other computation methods can be utilized to identify samples that have clear tri-nucleotide periodicity, the samples can be optionally utilized in additional computations methods such as PRICE, as discussed herein. In embodiments, the ORFs can be predicted independently from different levels taken from reads in, for example, each sample (leaves), multiple samples of the same tissue (branches), and reads of all samples (root). Prediction of nuORFs may be predicted at nodes in root, leaves and branches, or exclusively at nodes in leaves, or exclusively at branches. Reads can be pooled across all samples even if a particular sample may have insufficient reads. Computational methods such as PRICE can also be utilized, as described in Erhard et al., Nat. Methods. 2018 May; 15(5): 363-366; doi: 10.1038/nmeth.4631. PRICE modeling can be used to address experimental noise to accurately resolve overlapping short open reading frames (sORFs) and non-canonical translation initiation. See, e.g. Erhard (2018) at FIG. 1, incorporated by reference, for a generalized approach. Predicted ORF results from one or more approaches can optionally be combined. In one aspect, methods can be performed sequentially, utilizing results from a bioinformatic method in other bioinformatic method, particularly using the same reference transcriptome. In embodiments, nuORFs may overlap, including at 5′ UTRs and annotated ORFs that are difficult or impossible to identify from RNA-Seq. ORFs can be canonical, e.g. identical to a protein-coding ORF annotated in a reference, truncated with a predicted start codon 3′ downstream, or extended with a predicted start 5′ extended, or may be entirely contained within but out-of-frame relative to an annotated ORF. ORFs can be entirely contained in the 5′ UTR or 3′ UTR of a protein coding transcript, or overlap with a start codon in the 5′ UTR, or a stop codon in the 3′ UTR. Importantly, inclusion of overlapping nuORFs in the data provides a methodology that allows for discovery of additional novel ORFs. An alternate method for identifying neoantigens is described in PCT/US2019/054365.

In embodiments, predicted ORFs of a length longer than 15, 16, 17, 18, 19, 20, 21, 22, 23, 24 or 25 nucleotides long can be retained for further analysis, in an aspect, the predicted ORFs or 21 nt or longer can be retained for further analysis, including database generation. Advantageously, methods disclosed herein can allow nuORF predictions to be more sample and tissue specific than annotated ORFs.

RibORF and other computation methods can be utilized to identify samples that have clear tri-nucleotide periodicity. In embodiments, the ORFs can be predicted independently from different levels taken from reads in, for example, each sample, multiple samples of the same tissue, and reads of all samples. Reads can be pooled across all samples even if a particular sample may have insufficient reads. A final set of neoantigens can be generated in methods by searching the nuORF database for predicted nuORFs matching data in a MHC I immunopeptidome data set, wherein the identified predicted nuORFs comprise the final neoantigen set.

In another aspect, the invention provides an enriched population of translated neoantigens obtained by the enhanced Ribo-Seq method described herein. The enriched population of translated neoantigens can be synthesized by conventional peptide synthesis methods or can be stored in a digital library or database. The enriched population of translated neoantigens stored in a digital library or database can be used for making comparisons against, e.g., whole genome sequencing or transcription analysis. The neoantigens and neopolypeptides prepared can be specific to a subject and compared to the database. In an aspect, the subject has cancer, a pathogenic disorder, or a genetic disorder.

The method may further comprise searching an annotated proteome database for ORFs in the annotated proteome database matching data in the WIC I immunopeptidome dataset. The method may also further comprise selecting presented nuORFs identified in the nuORF database but not the annotated proteome database to generate the final set of neoantigens. In an aspect, the WIC I immunopeptidome data is obtained on biological samples from a subject to be treated. In an embodiment, the immunopeptidome data is mass spectroscopy data.

In some embodiments, measuring the level of expression of the at least one or more unique second markers includes subjecting each sample or a portion thereof to metaribosome profiling or ribosome profiling (Ribo-Seq) (see, e.g., Ingolia, N. T., S. Ghaemmaghami, J. R. Newman, and J. S. Weissman, 2009, “Genome-wide analysis in vivo of translation with nucleotide resolution using ribosome profiling” Science 324:218-223; Ingolia, N. T., 2014, “Ribosome profiling: new views of translation, from single codons to genome scale” Nat. Rev. Genet 15:205-213; each of which is incorporated by reference in it entirety for all purposes). Ribo-seq is a molecular technique that can be used to determine in vivo protein synthesis at the genome-scale. This method directly measures which transcripts are being actively translated via footprinting ribosomes as they bind and interact with mRNA. The bound mRNA regions are then processed and subjected to high-throughput sequencing reactions. Ribo-seq has been shown to have a strong correlation with quantitative proteomics (see, e.g., Li, G. W., D. Burkhardt, C. Gross, and J. S. Weissman. 2014 “Quantifying absolute protein synthesis rates reveals principles underlying allocation of cellular resources” Cell 157:624-635, the entirety of which is herein expressly incorporated by reference).

WGS (also known as full genome sequencing, complete genome sequencing, or entire genome sequencing), is a process that determines the complete DNA sequence of a subject. In some aspects, WGS, as embodied in the methods of Ng and Kirkness, Methods Mol. Biol.; 628:215-26 (2010), may be employed with the methods of the present disclosure to detect CLL mutations in a sample.

WES (also known as exome sequencing, or targeted exome capture), is an efficient strategy to selectively sequence the coding regions of the genome of a subject as a cheaper but still effective alternative to WGS. As exemplified by the methods of Gnirke et al., Nature Biotechnology 27, 182-189 (2009), WES of tumors and their patient-matched normal samples is an affordable, rapid and comprehensive technology for detecting somatic coding mutations.

Deep sequencing methods provide for greater coverage (depth) in targeted sequencing approaches. “Deep sequencing,” “deep coverage,” or “depth” refers to having a high amount of coverage for every nucleotide being sequenced. The high coverage allows not only the detection of nucleotide changes, but also the degree of heterogeneity at every single base in a genetic sample. Moreover, deep sequencing is able to simultaneously detect small indels and large deletions, map exact breakpoints, calculate deletion heterogeneity, and monitor copy number changes. In some aspects, deep sequencing strategies, as provided by Myllykangas and Ji, Biotechnol Genet Eng Rev. 27:135-58 (2010), may be employed with the methods of the present disclosure.

Using the enhanced Ribo-Seq method described herein, in one aspect the invention provides a method for preparing a neoantigen for an immunogenic pharmaceutical composition, wherein the neoantigen is specific to a subject that has a cancer, wherein the neoantigen is specific to the subject's cancer, wherein the neoantigen binds to an HLA protein of the subject, and wherein the neoantigen comprises a subject-specific mutated amino acid sequence expressed by cancer cells of the subject but not expressed by non-cancer cells of the subject that is encoded by a mutated coding sequence of the subject's cancer cells (neo-ORF), said method for preparing a neoantigen comprising comparing cancer and non-cancer cellular translation products of the subject comprising: (a) extracting from cancer cells of the subject a sample of ribosomes containing ribosome-protected mRNA fragments (RPFs), (b) removing rRNA from the RPFs to obtain rRNA-removed RPFs, (c) purifying the rRNA-removed RPFs to obtain purified RPFs, (d) preparing a library of purified circular DNA (cDNA) from the purified RPFs, said purified cDNA having open reading frames (ORFs), and (e) identifying the neo-ORF of the purified cDNA that encodes the neoantigen from cancer cells by comparing ORFs of purified cDNA with ORFs of non-cancer cells.

The methods described herein for preparing a neoantigen comprise extracting from cancer cells of the subject a sample of ribosomes containing ribosome-protected mRNA fragments (RPFs). In an aspect, extracting from cancer cells of the subject a sample of ribosomes containing ribosome-protected mRNA fragments (RPFs) (step a) comprises lysing the cancer cells to obtain a lysate and separating RPFs from the lysate. Separating RPFs from the lysate may comprise column chromatography.

In one embodiment, identifying the neo-ORF of the purified cDNA that encodes the neoantigen from cancer cells by comparing ORFs of purified cDNA with ORFs of non-cancer cells (step (e)) comprises (a′) extracting from non-cancer cells of the subject a sample of ribosomes containing ribosome-protected mRNA fragments (nccRPFs), (b′) removing rRNA from the nccRPFs to obtain rRNA-removed nccRPFs, (c′) purifying the rRNA-removed nccRPFs to obtain purified nccRPFs, (d′) preparing a library of purified circular DNA (cDNA) from the purified nccRPFs, and (e′) identifying ORFs of the purified cDNA from the purified nccRPFs and comparing those ORFs with ORFs of the purified cDNA of step (d).

In yet another embodiment, purifying the rRNA-removed RPFs to obtain purified RPFs (step (c)) and/or preparing a library of purified circular DNA (cDNA) from the purified RPFs, said purified cDNA having open reading frames (ORFs) (step (d)) comprises gel electrophoresis.

In another embodiment, preparing a library of purified circular DNA (cDNA) (having open reading frames (ORFs)) from the purified RPFs (step (d)) includes amplifying cDNA. The amplification can comprise between 8 and 10 amplification cycles.

In some embodiments, removing rRNA from the RPFs to obtain rRNA-removed RPFs (step (b)) does not include quantifying the RPFs.

The invention provides a method for preparing a neopolypeptide, wherein the neopolypeptide is specific to a subject that has a genetic disorder, is specific to the subject's genetic disorder, and comprises a subject-specific mutated amino acid sequence of the subject's genetic disorder, said method comprising comparing genetic disorder and non-genetic disorder cellular translation products of the subject comprising (a) extracting from genetic disorder cells of the subject a sample of ribosomes containing ribosome-protected mRNA fragments (RPFs), (b) removing rRNA from the RPFs to obtain rRNA-removed RPFs, (c) purifying the rRNA-removed RPFs to obtain purified RPFs, (d) preparing a library of purified circular DNA (cDNA) from the purified RPFs, said purified cDNA having open reading frames (ORFs), and (e) identifying the neo-ORF of the purified cDNA that encodes the neopolypeptide of the genetic disorder by comparing ORFs of purified cDNA with ORFs of non-genetic disorder cells.

In some embodiments, the identifying the neo-ORF of the purified cDNA that encodes the neopolypeptide of the genetic disorder by comparing ORFs of purified cDNA with ORFs of non-genetic disorder cells step (step (e)) of the method for preparing a neopolypeptide comprises (a′) extracting from non-genetic disorder ribosome samples containing ribosome-protected mRNA fragments (ngdRPFs), (b′) removing rRNA from the ngdRPFs to obtain rRNA-removed ngdRPFs, (c′) purifying the rRNA-removed ngdRPFs to obtain purified ngdRPFs, (d′) preparing a library of purified circular DNA (cDNA) from the purified ngdRPFs, (e′) identifying ORFs of the purified cDNA from the purified ngdRPFs and comparing those ORFs with ORFs of the purified cDNA of step (d).

In some embodiments, the extracting from genetic disorder cells of the subject a sample of ribosomes containing ribosome-protected mRNA fragments (RPFs) step (step (a)) of the method for preparing a neopolypeptide comprises lysing the cells to obtain a lysate and separating RPFs from the lysate. RPFs can be separated from lysate by, e.g., centrifugation.

In some embodiment, the purifying the rRNA-removed RPFs to obtain purified RPFs (step (c)) and/or the preparing a library of purified circular DNA (cDNA) from the purified RPFs, said purified cDNA having open reading frames (ORFs) step (step (d)) of the method for preparing a neopolypeptide comprises gel electrophoresis.

In some embodiments, the preparing a library of purified circular DNA (cDNA) from the purified RPFs, said purified cDNA having open reading frames (ORFs) step (step (d)) of the method for preparing a neopolypeptide includes amplifying cDNA, preferably between 8 and 10 amplification cycles.

In some embodiments, the removing rRNA from the RPFs to obtain rRNA-removed RPFs (step (b)) of the method for preparing a neopolypeptide does not include quantifying the RPF s.

In some embodiments, extracting from genetic disorder cells of the subject a sample of ribosomes containing ribosome-protected mRNA fragments (RPFs) step (step (a)) of the method for preparing a neopolypeptide includes centrifugation and/or column chromatography to separate RPF s.

The invention provides a method for preparing a neopolypeptide, wherein the neopolypeptide is specific to a subject that has a pathogenic disorder, is specific to the subject's pathogenic disorder, and comprises a subject-specific mutated amino acid sequence of the subject's pathogenic disorder, said method comprising comparing pathogenic disorder and non-pathogenic disorder cellular translation products of the subject comprising (a) extracting from pathogenic disorder cells of the subject a sample of ribosomes containing ribosome-protected mRNA fragments (RPFs), (b) removing rRNA from the RPFs to obtain rRNA-removed RPFs, (c) purifying the rRNA-removed RPFs to obtain purified RPFs, (d) preparing a library of purified circular DNA (cDNA) from the purified RPFs, said purified cDNA having open reading frames (ORFs), and (e) identifying the neo-ORF of the purified cDNA that encodes the neopolypeptide of the pathogenic disorder by comparing ORFs of purified cDNA with ORFs of non-pathogenic disorder cells.

In some embodiments, the identifying the neo-ORF of the purified cDNA that encodes the neopolypeptide of the pathogenic disorder by comparing ORFs of purified cDNA with ORFs of non-pathogenic disorder cells step (step (e)) comprises (a′) extracting from non-pathogenic disorder ribosome samples containing ribosome-protected mRNA fragments (npdRPFs), (b′) removing rRNA from the npdRPFs to obtain rRNA-removed npdRPFs, (c′) purifying the rRNA-removed npdRPFs to obtain purified npdRPFs, (d′) preparing a library of purified circular DNA (cDNA) from the purified npdRPFs, (e′) identifying ORFs of the purified cDNA from the purified npdRPFs and comparing those ORFs with ORFs of the purified cDNA of step (d).

In some embodiments, the extracting from pathogenic disorder cells of the subject a sample of ribosomes containing ribosome-protected mRNA fragments (RPFs) (step (a)) comprises lysing the cells to obtain a lysate and separating RPFs from the lysate. The RPFs can be separated by, e.g., centrifugation.

In some embodiments, the purifying the rRNA-removed RPFs to obtain purified RPFs step (step (c)) and/or the preparing a library of purified circular DNA (cDNA) from the purified RPFs, said purified cDNA having open reading frames (ORFs) (step (d)) of the method for preparing a neopolypeptide comprises gel electrophoresis.

In some embodiments, the preparing a library of purified circular DNA (cDNA) from the purified RPFs, said purified cDNA having open reading frames (ORFs) step (step (d)) includes amplifying cDNA. Amplification of cDNA can comprise between 8 and 10 amplification cycles.

In some embodiments, the removing rRNA from the RPFs to obtain rRNA-removed RPFs step (step (b)) does not include quantifying the RPFs.

In some embodiments, the extracting from pathogenic disorder cells of the subject a sample of ribosomes containing ribosome-protected mRNA fragments (RPFs) step (step (a)) includes centrifugation and/or column chromatography to separate RPFs.

In some embodiments, the neopolypeptide has a length of 8 or greater than 8 or 10 or greater than 10 or 15 or greater than 15 or 20 or greater than 20 or 8 to 50 or 15 to 30 or 20 to 40 amino acids.

As described herein, there is a large body of evidence in both animals and humans that mutated epitopes are effective in inducing an immune response and that cases of spontaneous tumor regression or long term survival correlate with CD8+ T-cell responses to mutated epitopes (Buckwalter and Srivastava P K. “It is the antigen(s), stupid” and other lessons from over a decade of vaccitherapy of human cancer. Seminars in immunology 20:296-300 (2008); Karanikas et al, High frequency of cytolytic T lymphocytes directed against a tumor-specific mutated antigen detectable with HLA tetramers in the blood of a lung carcinoma patient with long survival. Cancer Res. 61:3718-3724 (2001); Lennerz et al, The response of autologous T cells to a human melanoma is dominated by mutated neoantigens. Proc Natl Acad Sci USA.102:16013 (2005)) and that “immunoediting” can be tracked to alterations in expression of dominant mutated antigens in mice and man (Matsushita et al, Cancer exome analysis reveals a T-cell-dependent mechanism of cancer immunoediting Nature 482:400 (2012); DuPage et al, Expression of tumor-specific antigens underlies cancer immunoediting Nature 482:405 (2012); and Sampson et al, Immunologic escape after prolonged progression-free survival with epidermal growth factor receptor variant III peptide vaccination in patients with newly diagnosed glioblastoma J Clin Oncol. 28:4722-4729 (2010)).

Sequencing technology has revealed that each tumor contains multiple, patient-specific mutations that alter the protein coding content of a gene. Such mutations create altered proteins, ranging from single amino acid changes (caused by missense mutations) to addition of long regions of novel amino acid sequence due to frame shifts, read-through of termination codons or translation of intron regions (novel open reading frame mutations; neoORFs). These mutated proteins are valuable targets for the host's immune response to the tumor as, unlike native proteins, they are not subject to the immune-dampening effects of self-tolerance. Therefore, mutated proteins are more likely to be immunogenic and are also more specific for the tumor cells compared to normal cells of the patient.

### Libraries

The invention provides a method for preparing a neoantigen library, wherein the library comprises at least one set of neoantigens or neoantigen molecular information, and the neoantigens of a set are specific to a subject that has a disease or disorder as disclosed elsewhere herein, In an example, to subject has cancer, and the neoantigens of a set are specific to the subject's cancer, binds to an HLA protein of the subject, and comprises a subject-specific mutated amino acid sequence expressed by cancer cells of the subject but not expressed by non-cancer cells of the subject, encoded by a mutated coding sequence of the subject's cancer cells (neo-ORF), said method comprising comparing cancer and non-cancer cellular translation products of the subject comprising (a) extracting from cancer cells of the subject a sample of ribosomes containing ribosome-protected mRNA fragments (RPFs), (b) removing rRNA from the RPFs to obtain rRNA-removed RPFs, (c) purifying the rRNA-removed RPFs to obtain purified RPFs, (d) preparing a library of purified circular DNA (cDNA) from the purified RPFs, said purified cDNA having open reading frames (ORFs), and (e) identifying the neo-ORF of the purified cDNA that encodes the neoantigen from cancer cells by comparing ORFs of purified cDNA with ORFs of non-cancer cells.

In some embodiments, the identifying the neo-ORF of the purified cDNA that encodes the neoantigen from cancer cells by comparing ORFs of purified cDNA with ORFs of non-cancer cells step (step (e)) comprises (a′) extracting from non-cancer cells of the subject a sample of ribosomes containing ribosome-protected mRNA fragments (nccRPFs), (b′) removing rRNA from the nccRPFs to obtain rRNA-removed nccRPFs, (c′) purifying the rRNA-removed nccRPFs to obtain purified nccRPFs, (d′) preparing a library of purified circular DNA (cDNA) from the purified nccRPFs, (e′) identifying ORFs of the purified cDNA from the purified nccRPFs and comparing those ORFs with ORFs of the purified cDNA of step (d).

In some embodiments, the extracting from cancer cells of the subject a sample of ribosomes containing ribosome-protected mRNA fragments (RPFs) step (step (a)) comprises lysing the cancer cells to obtain a lysate and separating RPFs from the lysate.

In some embodiments, the purifying the rRNA-removed RPFs to obtain purified RPFs step (step (c)) and/or the preparing a library of purified circular DNA (cDNA) from the purified RPFs, said purified cDNA having open reading frames (ORFs) step (step (d)) comprises gel electrophoresis.

In some embodiments, the preparing a library of purified circular DNA (cDNA) from the purified RPFs, said purified cDNA having open reading frames (ORFs) step (step (d)) includes amplifying cDNA, which can comprise between 8 and 10 amplification cycles.

In some embodiments, the removing rRNA from the RPFs to obtain rRNA-removed RPFs step (step (b)) does not include quantifying the RPFs.

In some embodiments, the cancer is chronic lymphocyte leukemia and/or the non-cancer cells are B cells.

In some embodiments, the cancer is melanoma and/or the non-cancer cells are melanocytes.

In some embodiments, the extracting from cancer cells of the subject a sample of ribosomes containing ribosome-protected mRNA fragments (RPFs) step (step (a)) includes centrifugation and/or column chromatography to separate RPFs.

In some embodiments, the ORFs of non-cancer cells are from a whole genome sequencing analysis.

In some embodiments, the neoantigen binds to the HLA protein of the subject with an IC₅₀ of less than or about 50, less than or about 100, less than or about 250 or less than or about 500 nM and a greater affinity than a corresponding wild-type peptide.

In some embodiments, the neoantigen has a length of 8 or greater than 8 or 10 or greater than 10 or 15 or greater than 15 or 20 or greater than 20 or 8 to 50 or 15 to 30 or 20 to 40 amino acids.

In some embodiments, the HLA protein of the subject is a class I HLA protein.

In some embodiments, the HLA protein of the subject is a class II HLA protein.

In some embodiments, the neoantigen elicits an immune response comprising a cytotoxic T cell response, a CD4 or helper T cell response, a CD8 or suppressor T cell response or a combination thereof.

In some embodiments, the cancer is a solid tumor, hematological cancer, breast cancer, ovarian cancer, prostate cancer, lung cancer, kidney cancer, gastric cancer, colon cancer, testicular cancer, head and neck cancer, pancreatic cancer, brain cancer, bladder cancer, melanoma, lymphoma or leukemia.

In some embodiments, the neoantigen or a portion thereof is presented to the subject's immune system by MHC I molecules or MHC II molecules.

In some embodiments, the method for preparing a neoantigen library comprises synthesizing the neoantigen.

In some embodiments, the library comprises neoantigen molecular information or more than one set of neoantigens.

The invention provides a library of neoantigen molecules from ribosomal or translational analysis of cancer cells or from any of the methods described herein.

The invention provides a method for preparing a neopolypeptide library, wherein the library comprises at least one set of neopolypeptide or neopolypeptide molecular information, and the neopolypeptides of a set are specific to a subject that has a genetic disorder, specific to the subject's genetic disorder, and comprises a subject-specific mutated amino acid sequence of the subject's genetic disorder, said method comprising comparing genetic disorder and non-genetic disorder cellular translation products of the subject comprising (a) extracting from genetic disorder cells of the subject a sample of ribosomes containing ribosome-protected mRNA fragments (RPFs), (b) removing rRNA from the RPFs to obtain rRNA-removed RPFs, (c) purifying the rRNA-removed RPFs to obtain purified RPFs, (d) preparing a library of purified circular DNA (cDNA) from the purified RPFs, said purified cDNA having open reading frames (ORFs), and (e) identifying the neo-ORF of the purified cDNA that encodes the neopolypeptide of the genetic disorder by comparing ORFs of purified cDNA with ORFs of non-genetic disorder cells.

In some embodiments, the identifying the neo-ORF of the purified cDNA that encodes the neopolypeptide of the genetic disorder by comparing ORFs of purified cDNA with ORFs of non-genetic disorder cells step (step (e)) comprises (a′) extracting from non-genetic disorder ribosome samples containing ribosome-protected mRNA fragments (ngdRPFs), (b′) removing rRNA from the ngdRPFs to obtain rRNA-removed ngdRPFs, (c′) purifying the rRNA-removed ngdRPFs to obtain purified ngdRPFs, (d′) preparing a library of purified circular DNA (cDNA) from the purified ngdRPFs, (e′) identifying ORFs of the purified cDNA from the purified ngdRPFs and comparing those ORFs with ORFs of the purified cDNA of step (d).

In some embodiments, the extracting from genetic disorder cells of the subject a sample of ribosomes containing ribosome-protected mRNA fragments (RPFs) step (step (a)) comprises lysing the cells to obtain a lysate and separating RPFs from the lysate.

In some embodiments, the purifying the rRNA-removed RPFs to obtain purified RPFs step (step (c)) and/or the preparing a library of purified circular DNA (cDNA) from the purified RPFs, said purified cDNA having open reading frames (ORFs) step (step (d)) comprises gel electrophoresis.

In some embodiments, the preparing a library of purified circular DNA (cDNA) from the purified RPFs, said purified cDNA having open reading frames (ORFs) step (step (d)) includes amplifying cDNA. The cDNA amplification can comprise between 8 and 10 amplification cycles.

In some embodiments, the removing rRNA from the RPFs to obtain rRNA-removed RPFs step (step (b)) does not include quantifying the RPFs.

In some embodiments, the extracting from genetic disorder cells of the subject a sample of ribosomes containing ribosome-protected mRNA fragments (RPFs) step (step (a)) includes centrifugation and/or column chromatography to separate RPFs.

In some embodiments, the neopolypeptide has a length of 8 or greater than 8 or 10 or greater than 10 or 15 or greater than 15 or 20 or greater than 20 or 8 to 50 or 15 to 30 or 20 to 40 amino acids.

In some embodiments, the method for preparing a neopolypeptide library comprises synthesizing the neopolypeptide.

In some embodiments, the library comprises neopolypeptide molecular information.

In some embodiments, the library comprises more than one set of neopolypeptides or neopolypeptide molecular information.

The invention provides a library of neopolypeptide molecules from any of the methods described herein.

The invention provides any method or library or system described herein wherein the pathogen is a virus or a bacteria or a pathogen or fungi comprising AIDS (acquired immunodeficiency syndrome), Argentine hemorrhagic fever, Astrovirus infection, BK virus infection, Bolivian hemorrhagic fever, Brazilian hemorrhagic fever, Calicivirus infection (Norovirus and Sapovirus), chicken pox, Chikungunya, Colorado tick fever (CTF), common cold, Crimean-Congo hemorrhagic fever (CCHF), Cytomegalovirus infection, Dengue fever, Ebola hemorrhagic fever, Enterovirus infection, Erythema infectiosum (Fifth disease), Exanthem subitum (Sixth disease), Hand, foot and mouth disease (HFMD), Hantavirus Pulmonary Syndrome (HPS), Heartland virus disease, Hemorrhagic fever with renal syndrome (HFRS), Hendra virus infection, Hepatitis A, Hepatitis B, Hepatitis C, Hepatitis D, Hepatitis E, Herpes simplex, Human bocavirus infection, Human metapneumovirus infection, Human papillomavirus (HPV) infection, Human parainfluenza virus infection, Epstein-Barr virus infectious mononucleosis (Mono), influenza, Keratitis, Lassa fever, Lymphocytic choriomeningitis, Marburg hemorrhagic fever (MHF), Measles, Middle East respiratory syndrome (MERS), Meningitis, Molluscum contagiosum (MC), Monkeypox, Mumps, Nipah virus infection, Norovirus, Pneumonia, Poliomyelitis, Progressive multifocal leukoencephalopathy, Rabies, Respiratory syncytial virus infection, Rhinovirus infection, Rift Valley fever (RVF), Rotavirus infection, Rubella, SARS (Severe Acute Respiratory Syndrome), Shingles (Herpes zoster), Smallpox (Variola), Subacute sclerosing panencephalitis, Venezuelan equine encephalitis, Venezuelan hemorrhagic fever, West Nile Fever, Yellow fever, Zika fever, Acenetobacter infections, actinomycosis, Anaplasmosis, Anthrax, Arcanobacterium haemolyticum infection, Bacillus cereus infection, Bacterial pneumonia, Bacterial vaginosis, Bacteroides infection, Bartonellosis, botulism, Brucellosis, Bubonic plague, Burkholderia infection, Buruli ulcer, Campylobacteriosis, Carrion's disease, Cat-scratch disease, cellulitis, Chancroid, Chlamydia, Chlamydophila pneumoniae infection (Taiwan acute respiratory agent or TWAR), Cholera, Clostridium difficile colitis, Diphtheria, Ehrlichiosis, Enterococcus infection, Epidemic typhus, Food poisoning by Clostridium perfringens, Fusobacterium infection, Gas gangrene, Glanders, Gonorrhea, Granuloma inguinale (Donovanosis), Group A streptococcal infection, Group B streptococcal infection, Haemophilus influenzae infection, Helicobacter pylori infection, Hemolytic-uremic syndrome (HUS), Human ewingii ehrlichiosis, Human granulocytic anaplasmosis (HGA), Human monocytic ehrlichiosis, Keratitis, Kingella kingae infection, Legionellosis (Legionnaires' disease), Legionellosis (Pontiac fever), Leprosy, Leptospirosis, Listeriosis, Lyme disease (Lyme borreliosis), Melioidosis (Whitmore's disease), Meningitis, Meningococcal disease, Murine typhus (Endemic typhus), Mycoplasma pneumonia, Mycoplasma genitalium infection, Mycetoma (disambiguation), Neonatal conjunctivitis (Ophthalmia neonatorum), Nocardiosis, Pasteurellosis, Pelvic inflammatory disease (PID), Pertussis (Whooping cough), Plague, Pneumococcal infection, Pneumonia, Prevotella infection, Psittacosis, Q fever, Relapsing fever, Rickettsial infection, Rickettsialpox, Rocky Mountain spotted fever (RMSF), Salmonellosis, Scarlet fever, Shigellosis (Bacillary dysentery), Staphylococcal food poisoning, Staphylococcal infection, Syphilis, Tetanus (Lockjaw), Trachoma, Tuberculosis, Tularemia, Typhoid fever, Typhus fever, Ureaplasma urealyticum infection, Vibrio vulnificus infection, Vibrio parahaemolyticus enteritis, Yersinia pseudotuberculosis infection, Yersiniosis, African sleeping sickness (African trypanosomiasis), amebiasis, Angiostrongyliasis, Anisakiasis, Ascariasis, Babesiosis, Balantidiasis, Baylisascaris infection, Blastocystosis, Capillariasis, Chagas Disease (American trypanosomiasis), Clonorchiasis, Cryptosporidiosis, Cutaneous larva migrans (CLM), Cyclosporiasis, Cysticercosis, Dientamoebiasis, Diphyllobothriasis, Dracunculiasis, Echinococcosis, Enterobiasis (Pinworm infection), Fasciolasis, Fasciolopsiasis, Filariasis, Giardiasis, Gnathostomiasis, Hookworm infection, Hymenolepiasis, Isosporiasis, Keratitis, Leishmaniasis, Lymphatic filariasis (Elephantiasis), Malaria, Meningitis, Metagonimiasis, Myiasis, Onchocerciasis (River blindness), Opisthorchiasis, Paragonimiasis, Pediculosis capitis (Head lice), Pediculosis corporis (Body lice), Pediculosis pubis (Pubic lice, Crab lice), Pneumonia, Scabies, Schistosomiasis, Strongyloidiasis, Taeniasis, Toxocariasis (Ocular Larva Migrans (OLM)), Toxocariasis (Visceral Larva Migrans (VLM)), Toxoplasmosis, Trichinosis, Trichomoniasis, Trichuriasis (Whipworm infection), Aspergillosis, Black piedra, Blastomycosis, Candidiasis, Chromoblastomycosis, Chytridiomycosis, Coccidioidomycosis, Cryptococcosis, Geotrichosis, Histoplasmosis, Keratitis, Meningitis, Paracoccidioidomycosis (South American blastomycosis), Pneumocystis pneumonia (PCP), Pneumonia, Sporotrichosis, Tinea barbae (Barber's itch), Tinea capitis (Ringworm of the Scalp), Tinea corporis (Ringworm of the Body), Tinea cruris (Jock itch), Tinea manum (Ringworm of the Hand), Tinea nigra, Tinea pedis (Athlete's foot), Tinea unguium (Onychomycosis), Tinea versicolor (Pityriasis versicolor), Valley fever, White piedra (Tinea blanca), Zeaspora, or Zygomycosis.

The invention provides any method or library or system described herein wherein the genetic disorder is 1p36 deletion syndrome, 18p deletion syndrome, 21-hydroxylase deficiency, alpha 1-antitrypsin deficiency, AAA syndrome (achalasia-addisonianism-alacrima), Aarskog-Scott syndrome, ABCD syndrome, aceruloplasminemia, acheiropodia, achondrogenesis type II, achondroplasia, acute intermittent porphyria, adenylosuccinate lyase deficiency, adrenoleukodystrophy, alagille syndrome, ADULT syndrome, Aicardi-Goutieres syndrome, albinism, Alexander disease, alkaptonuria, alport syndrome, alternating hemiplegia of childhood, amyotrophic lateral sclerosis-Frontotemporal Dementia, Alstrom syndrome, alzheimer's disease, amelogenesis imperfect, aminolevulinic acid dehydratase deficiency porphyria, androgen insensitivity syndrome, angelman syndrome, apert syndrome, arthrogryposis-renal dysfunction-cholestasis syndrome, ataxia telangiectasia, axenfeld syndrome, Beare-Stevenson cutis gyrata syndrome, Beckwith-Wiedemann syndrome, Benjamin syndrome, biotinidase deficiency, Bjornstad syndrome, Bloom syndrome, Birt-Hogg-Dubé syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratodermasyndrome (SEDNIK), Cystic fibrosis, Charcot-Marie-Tooth disease, CHARGE syndrome, Chédiak-Higashi syndrome, Cleidocranial dysostosis, Cockayne syndrome, Coffin-Lowry syndrome, Cohen syndrome, collagenopathy, types II and XI, Congenital insensitivity to pain with anhidrosis (CIPA), Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Down Syndrome, Di George's syndrome, multiple types of Distal hereditary motor neuropathies, Dravet syndrome, Edwards Syndrome, Ehlers-Danlos syndrome, Emery-Dreifuss syndrome, Erythropoietic protoporphyria, Fanconi anemia (FA), Fabry disease, factor V Leiden thrombophilia, Fatal Familial Insomnia, familial adenomatous polyposis, familial dysautonomia, familial Creutzfeld-Jakob Disease, Feingold syndrome, FG syndrome, Fragile X syndrome, Friedreich's ataxia, G6PD deficiency, galactosemia, Gaucher disease, Gerstmann-Sträussler-Scheinker Syndrome, Gillespie syndrome, type I and type 2 Glutaric aciduria, GRACILE syndrome, Griscelli syndrome, Hailey-Hailey disease, Harlequin type ichthyosis, heteritary Hemochromatosis, hemophilia, Hepatoerythropoietic porphyria, Hereditary coproporphyria, Hereditary hemorrhagic telangiectasia (Osler-Weber-Rendu syndrome), Hereditary Inclusion Body Myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, homocystinuria, Huntington's disease, Hunter syndrome, Hurler syndrome, Hutchinson-Gilford progeria syndrome, Hyperlysinemia, primary hyperoxaluria, hyperphenylalaninemia, Hypoalphalipoproteinemia (Tangier disease), Hypochondrogenesis, Hypochondroplasia, Immunodeficiency, centromere instability and facial anomalies syndrome (ICF syndrome), Incontinentia pigmenti, Ischiopatellar dysplasia, Isodicentric 15, Jackson-Weiss syndrome, Joubert syndrome, Juvenile Primary Lateral Sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Lynch Syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, familial Mediterranean fever, Menkes disease, Methemoglobinemia, methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Duchenne and becker type Muscular dystrophy, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, osteogenesis imperfecta, Pantothenate kinase-associated neurodegeneration, Patau Syndrome (Trisomy 13), PCC deficiency (propionic acidemia), Porphyria cutanea tarda (PCT), Pendred syndrome, Peutz-Jeghers syndrome, Pfeiffer syndrome, phenylketonuria, Pipecolic acidemia, Pitt-Hopkins syndrome, Polycystic kidney disease, Polycystic Ovarian Syndrome (PCOS), porphyria, Prader-Willi syndrome, Primary ciliary dyskinesia (PCD), primary pulmonary hypertension, protein C deficiency, protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, spondyloepiphyseal dysplasia congenita (SED), Shprintzen-Goldberg syndrome, sickle cell anemia, Siderius X-linked mental retardation syndrome, Sideroblastic anemia, Sly syndrome, Smith-Lemli-Opitz syndrome, Smith Magenis Syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous Sclerosis Complex (TSC), Turner syndrome, Usher syndrome, Variegate porphyria, von Hippel-Lindau disease, Waardenburg syndrome, Weissenbacher-Zweymülller syndrome, Williams syndrome, Wilson disease, Woodhouse-Sakati syndrome, Wolf-Hirschhorn syndrome, Xeroderma pigmentosum, X-linked mental retardation and macroorchidism (fragile X syndrome), X-linked spinal-bulbar muscle atrophy (spinal and bulbar muscular atrophy), Xp11.22 deletion, X-linked severe combined immunodeficiency (X-SCID), X-linked sideroblastic anemia (XLSA), 47,XXX (triple X syndrome), XXXX syndrome (48, XXXX), XXXXX syndrome (49, XXXXX), XYY syndrome (47,XYY), or Zellweger syndrome.

### Methods of Treatment

The present invention provides methods of inducing a neoplasia/tumor specific immune response in a subject, vaccinating against a neoplasia/tumor, treating and or alleviating a symptom of cancer in a subject by administering the subject a plurality of neoantigenic peptides or composition of the invention. Adjuvants and combination therapies are contemplated for use with the present invention.

According to the invention, the herein-described neoplasia vaccine or immunogenic composition may be used for a patient that has been diagnosed as having cancer, or at risk of developing cancer. The claimed combination of the invention is administered in an amount sufficient to induce a CTL response. In an aspect, the method of treatment comprises administering any of the immunogenic compositions comprising at least one neoantigen obtained from the methods described herein.

In some embodiments, the method of treating a subject having a cancer in need of such treatment further comprises administering an anti-immunosuppressive agent or an anti-immunostimulatory agent or another antineoplastic agent or administering the immunogenic composition comprising at least one neoantigen in conjunction with another cancer therapy. The anti-immunosuppressive agent or the anti-immunostimulatory agent may be selected from the group consisting of an anti-CTLA agent, an anti-PD-1 agent, an anti-PD-L1 agent, an anti-CD25, an IDO inhibitor and combinations thereof. The other cancer therapy may comprise surgery.

The invention provides a method of treating a subject having a cancer in need of such treatment comprising administering the subject's dendritic cells pulsed with neoantigens as described in, e.g., Carreno et al., Science, Vol. 348, Issue 6236, pp. 803-808, wherein the neoantigens are prepared or identified by methods described herein.

The invention provides a method of treating a subject having a cancer in need of such treatment comprising administering the subject T cells isolated from the patient that have been activated and expanded ex vivo in the presence of the neoantigens identified by the methods described herein. (See Tran et al., Cancer immunotherapy based on mutation-specific CD4+ T cells in a patient with epithelial cancer, Science, Vol. 344, Issue 6184, pp. 641-645, 9 May 2014; Stevanovic et al., Landscape of immunogenic tumor antigens in successful immunotherapy of virally induced epithelial cancer, Science, Apr. 14, 2016, 356(6334):200-205).

The invention provides a method of treating a subject having a cancer in need of such treatment comprising administering to the subject a neoantigen vaccine comprising the neoantigens identified or prepared by the methods described herein.

Indications

Examples of cancers and cancer conditions that can be treated with the therapy of this document include, but are not limited to a patient in need thereof that has been diagnosed as having cancer, or at risk of developing cancer. The subject may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites. Thus the population of subjects described herein may be suffering from one of the above cancer types. In other embodiments, the population of subjects may be all subjects suffering from solid tumors, or all subjects suffering from liquid tumors.

Of special interest is the treatment of Non-Hodgkin's Lymphoma (NHL), clear cell Renal Cell Carcinoma (ccRCC), metastatic melanoma, sarcoma, leukemia or a cancer of the bladder, colon, brain, breast, head and neck, endometrium, lung, ovary, pancreas or prostate. In certain embodiments, the melanoma is high risk melanoma.

Cancers that can be treated using the therapy described herein may include among others cases which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

In particular embodiments, the treatments disclosed herein can be utilized for genetic disorders. In an aspect, the treatment comprises a neopolypeptide. In some embodiments, the neopolypeptide has a length of 8 or greater than 8 or 10 or greater than 10 or 15 or greater than 15 or 20 or greater than 20 or 8 to 50 or 15 to 30 or 20 to 40 amino acids.

In some embodiments, the genetic disorder comprises 1p36 deletion syndrome, 18p deletion syndrome, 21-hydroxylase deficiency, alpha 1-antitrypsin deficiency, AAA syndrome (achalasia-addisonianism-alacrima), Aarskog-Scott syndrome, ABCD syndrome, aceruloplasminemia, acheiropodia, achondrogenesis type II, achondroplasia, acute intermittent porphyria, adenylosuccinate lyase deficiency, adrenoleukodystrophy, alagille syndrome, ADULT syndrome, Aicardi-Goutieres syndrome, albinism, Alexander disease, alkaptonuria, alport syndrome, alternating hemiplegia of childhood, amyotrophic lateral sclerosis-Frontotemporal Dementia, Alstrom syndrome, alzheimer's disease, amelogenesis imperfect, aminolevulinic acid dehydratase deficiency porphyria, androgen insensitivity syndrome, angelman syndrome, apert syndrome, arthrogryposis-renal dysfunction-cholestasis syndrome, ataxia telangiectasia, axenfeld syndrome, Beare-Stevenson cutis gyrata syndrome, Beckwith-Wiedemann syndrome, Benjamin syndrome, biotinidase deficiency, Björnstad syndrome, Bloom syndrome, Birt-Hogg-Dubé syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chédiak-Higashi syndrome, Cleidocranial dysostosis, Cockayne syndrome, Coffin-Lowry syndrome, Cohen syndrome, collagenopathy, types II and XI, Congenital insensitivity to pain with anhidrosis (CIPA), Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Darier's disease, Dent's disease (Genetic hypercalciuria), Denys-Drash syndrome, De Grouchy syndrome, Down Syndrome, Di George's syndrome, multiple types of Distal hereditary motor neuropathies, Dravet syndrome, Edwards Syndrome, Ehlers-Danlos syndrome, Emery-Dreifuss syndrome, Erythropoietic protoporphyria, Fanconi anemia (FA), Fabry disease, factor V Leiden thrombophilia, Fatal Familial Insomnia, familial adenomatous polyposis, familial dysautonomia, familial Creutzfeld-Jakob Disease, Feingold syndrome, FG syndrome, Fragile X syndrome, Friedreich's ataxia, G6PD deficiency, galactosemia, Gaucher disease, Gerstmann-Sträussler-Scheinker Syndrome, Gillespie syndrome, type I and type 2 Glutaric aciduria, GRACILE syndrome, Griscelli syndrome, Hailey-Hailey disease, Harlequin type ichthyosis, heteritary Hemochromatosis, hemophilia, Hepatoerythropoietic porphyria, Hereditary coproporphyria, Hereditary hemorrhagic telangiectasia (Osler-Weber-Rendu syndrome), Hereditary Inclusion Body Myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, homocystinuria, Huntington's disease, Hunter syndrome, Hurler syndrome, Hutchinson-Gilford progeria syndrome, Hyperlysinemia, primary hyperoxaluria, hyperphenylalaninemia, Hypoalphalipoproteinemia (Tangier disease), Hypochondrogenesis, Hypochondroplasia, Immunodeficiency, centromere instability and facial anomalies syndrome (ICF syndrome), Incontinentia pigmenti, Ischiopatellar dysplasia, Isodicentric 15, Jackson-Weiss syndrome, Joubert syndrome, Juvenile Primary Lateral Sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, Li-Fraumeni syndrome, Lynch Syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, familial Mediterranean fever, Menkes disease, Methemoglobinemia, methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Duchenne and becker type Muscular dystrophy, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, osteogenesis imperfecta, Pantothenate kinase-associated neurodegeneration, Patau Syndrome (Trisomy 13), PCC deficiency (propionic acidemia), Porphyria cutanea tarda (PCT), Pendred syndrome, Peutz-Jeghers syndrome, Pfeiffer syndrome, phenylketonuria, Pipecolic acidemia, Pitt-Hopkins syndrome, Polycystic kidney disease, Polycystic Ovarian Syndrome (PCOS), porphyria, Prader-Willi syndrome, Primary ciliary dyskinesia (PCD), primary pulmonary hypertension, protein C deficiency, protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, spondyloepiphyseal dysplasia congenita (SED), Shprintzen-Goldberg syndrome, sickle cell anemia, Siderius X-linked mental retardation syndrome, Sideroblastic anemia, Sly syndrome, Smith-Lemli-Opitz syndrome, Smith Magenis Syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous Sclerosis Complex (TSC), Turner syndrome, Usher syndrome, Variegate porphyria, von Hippel-Lindau disease, Waardenburg syndrome, Weissenbacher-Zweymüller syndrome, Williams syndrome, Wilson disease, Woodhouse-Sakati syndrome, Wolf-Hirschhorn syndrome, Xeroderma pigmentosum, X-linked mental retardation and macroorchidism (fragile X syndrome), X-linked spinal-bulbar muscle atrophy (spinal and bulbar muscular atrophy), Xp11.22 deletion, X-linked severe combined immunodeficiency (X-SCID), X-linked sideroblastic anemia (XLSA), 47,XXX (triple X syndrome), XXXX syndrome (48, XXXX), XXXXX syndrome (49, XXXXX), XYY syndrome (47,XYY), or Zellweger syndrome.

Therapy can comprise treatment of a pathogenic disorder. In some embodiments, the pathogenic disorder comprises: a viral or bacterial or parasitic or fungi pathogenic disorder, or a viral disorder comprising AIDS (acquired immunodeficiency syndrome), Argentine hemorrhagic fever, Astrovirus infection, BK virus infection, Bolivian hemorrhagic fever, Brazilian hemorrhagic fever, Calicivirus infection (Norovirus and Sapovirus), chicken pox, Chikungunya, Colorado tick fever (CTF), common cold, Crimean-Congo hemorrhagic fever (CCHF), Cytomegalovirus infection, Dengue fever, Ebola hemorrhagic fever, Enterovirus infection, Erythema infectiosum (Fifth disease), Exanthem subitum (Sixth disease), Hand, foot and mouth disease (HFMD), Hantavirus Pulmonary Syndrome (HPS), Heartland virus disease, Hemorrhagic fever with renal syndrome (HFRS), Hendra virus infection, Hepatitis A, Hepatitis B, Hepatitis C, Hepatitis D, Hepatitis E, Herpes simplex, Human bocavirus infection, Human metapneumovirus infection, Human papillomavirus (HPV) infection, Human parainfluenza virus infection, Epstein-Barr virus infectious mononucleosis (Mono), influenza, Keratitis, Lassa fever, Lymphocytic choriomeningitis, Marburg hemorrhagic fever (MHF), Measles, Middle East respiratory syndrome (MERS), Meningitis, Molluscum contagiosum (MC), Monkeypox, Mumps, Nipah virus infection, Norovirus, Pneumonia, Poliomyelitis, Progressive multifocal leukoencephalopathy, Rabies, Respiratory syncytial virus infection, Rhinovirus infection, Rift Valley fever (RVF), Rotavirus infection, Rubella, SARS (Severe Acute Respiratory Syndrome), Shingles (Herpes zoster), Smallpox (Variola), Subacute sclerosing panencephalitis, Venezuelan equine encephalitis, Venezuelan hemorrhagic fever, West Nile Fever, Yellow fever, Zika fever; or a bacterial disorder comprising Acenetobacter infections, actinomycosis, Anaplasmosis, Anthrax, Arcanobacterium haemolyticum infection, Bacillus cereus infection, Bacterial pneumonia, Bacterial vaginosis, Bacteroides infection, Bartonellosis, botulism, Brucellosis, Bubonic plague, Burkholderia infection, Buruli ulcer, Campylobacteriosis, Carrion's disease, Cat-scratch disease, cellulitis, Chancroid, Chlamydia, Chlamydophila pneumoniae infection (Taiwan acute respiratory agent or TWAR), Cholera, Clostridium difficile colitis, Diphtheria, Ehrlichiosis, Enterococcus infection, Epidemic typhus, Food poisoning by Clostridium perfringens, Fusobacterium infection, Gas gangrene, Glanders, Gonorrhea, Granuloma inguinale (Donovanosis), Group A streptococcal infection, Group B streptococcal infection, Haemophilus influenzae infection, Helicobacter pylori infection, Hemolytic-uremic syndrome (HUS), Human ewingii ehrlichiosis, Human granulocytic anaplasmosis (HGA), Human monocytic ehrlichiosis, Keratitis, Kingella kingae infection, Legionellosis (Legionnaires' disease), Legionellosis (Pontiac fever), Leprosy, Leptospirosis, Listeriosis, Lyme disease (Lyme borreliosis), Melioidosis (Whitmore's disease), Meningitis, Meningococcal disease, Murine typhus (Endemic typhus), Mycoplasma pneumonia, Mycoplasma genitalium infection, Mycetoma (disambiguation), Neonatal conjunctivitis (Ophthalmia neonatorum), Nocardiosis, Pasteurellosis, Pelvic inflammatory disease (PID), Pertussis (Whooping cough), Plague, Pneumococcal infection, Pneumonia, Prevotella infection, Psittacosis, Q fever, Relapsing fever, Rickettsial infection, Rickettsialpox, Rocky Mountain spotted fever (RMSF), Salmonellosis, Scarlet fever, Shigellosis (Bacillary dysentery), Staphylococcal food poisoning, Staphylococcal infection, Syphilis, Tetanus (Lockjaw), Trachoma, Tuberculosis, Tularemia, Typhoid fever, Typhus fever, Ureaplasma urealyticum infection, Vibrio vulnificus infection, Vibrio parahaemolyticus enteritis, Yersinia pseudotuberculosis infection, Yersiniosis; or a parasitic pathogenic disorder comprising African sleeping sickness (African trypanosomiasis), amebiasis, Angiostrongyliasis, Anisakiasis, Ascariasis, Babesiosis, Balantidiasis, Baylisascaris infection, Blastocystosis, Capillariasis, Chagas Disease (American trypanosomiasis), Clonorchiasis, Cryptosporidiosis, Cutaneous larva migrans (CLM), Cyclosporiasis, Cysticercosis, Dientamoebiasis, Diphyllobothriasis, Dracunculiasis, Echinococcosis, Enterobiasis (Pinworm infection), Fasciolasis, Fasciolopsiasis, Filariasis, Giardiasis, Gnathostomiasis, Hookworm infection, Hymenolepiasis, Isosporiasis, Keratitis, Leishmaniasis, Lymphatic filariasis (Elephantiasis), Malaria, Meningitis, Metagonimiasis, Myiasis, Onchocerciasis (River blindness), Opisthorchiasis, Paragonimiasis, Pediculosis capitis (Head lice), Pediculosis corporis (Body lice), Pediculosis pubis (Pubic lice, Crab lice), Pneumonia, Scabies, Schistosomiasis, Strongyloidiasis, Taeniasis, Toxocariasis (Ocular Larva Migrans (OLM)), Toxocariasis (Visceral Larva Migrans (VLM)), Toxoplasmosis, Trichinosis, Trichomoniasis, Trichuriasis (Whipworm infection); or a fungi disorder comprising Aspergillosis, Black piedra, Blastomycosis, Candidiasis, Chromoblastomycosis, Chytridiomycosis, Coccidioidomycosis, Cryptococcosis, Geotrichosis, Histoplasmosis, Keratitis, Meningitis, Paracoccidioidomycosis (South American blastomycosis), Pneumocystis pneumonia (PCP), Pneumonia, Sporotrichosis, Tinea barbae (Barber's itch), Tinea capitis (Ringworm of the Scalp), Tinea corporis (Ringworm of the Body), Tinea cruris (Jock itch), Tinea manum (Ringworm of the Hand), Tinea nigra, Tinea pedis (Athlete's foot), Tinea unguium (Onychomycosis), Tinea versicolor (Pityriasis versicolor), Valley fever, White piedra (Tinea blanca), Zeaspora, or Zygomycosis.

The therapy described herein is also applicable to the treatment of patients in need thereof who have not been previously treated.

The therapy described herein is also applicable where the subject has no detectable neoplasia but is at high risk for disease recurrence.

Also of special interest is the treatment of patients in need thereof who have undergone Autologous Hematopoietic Stem Cell Transplant (AHSCT), and in particular patients who demonstrate residual disease after undergoing AHSCT. The post-AHSCT setting is characterized by a low volume of residual disease, the infusion of immune cells to a situation of homeostatic expansion, and the absence of any standard relapse-delaying therapy. These features provide a unique opportunity to use the claimed neoplastic vaccine or immunogenic composition compositions to delay disease relapse.

Administering a Combination Therapy Consistent with Standard of Care

In another aspect, the therapy described herein provides selecting the appropriate point to administer a combination therapy in relation to and within the standard of care for the cancer being treated for a patient in need thereof. The studies described herein show that the combination therapy can be effectively administered even within the standard of care that includes surgery, radiation, or chemotherapy. The standards of care for the most common cancers can be found on the website of National Cancer Institute (www.cancer.gov/cancertopics). The standard of care is the current treatment that is accepted by medical experts as a proper treatment for a certain type of disease and that is widely used by healthcare professionals. Standard or care is also called best practice, standard medical care, and standard therapy. Standards of Care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The combination therapy described herein can be incorporated within the standard of care. The combination therapy described herein may also be administered where the standard of care has changed due to advances in medicine.

“Combination therapy” is intended to embrace administration of therapeutic agents (e.g. neoantigenic peptides described herein) in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of neoantigenic peptides administered at the same or different times, or they can be formulated as a single, co-formulated pharmaceutical composition comprising the peptides. As another example, a combination of the present invention (e.g., a pooled sample of tumor specific neoantigens) may be formulated as separate pharmaceutical compositions that can be administered at the same or different time. As used herein, the term “simultaneously” is meant to refer to administration of one or more agents at the same time. For example, in certain embodiments, the neoantigenic peptides are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

Incorporation of the combination therapy described herein may depend on a treatment step in the standard of care that can lead to activation of the immune system. Treatment steps that can activate and function synergistically with the combination therapy have been described herein. The therapy can be advantageously administered simultaneously or after a treatment that activates the immune system.

Incorporation of the combination therapy described herein may depend on a treatment step in the standard of care that causes the immune system to be suppressed. Such treatment steps may include irradiation, high doses of alkylating agents and/or methotrexate, steroids such as glucosteroids, surgery, such as to remove the lymph nodes, imatinib mesylate, high doses of TNF, and taxanes (Zitvogel et al., 2008). The combination therapy may be administered before such steps or may be administered after.

In one embodiment the combination therapy may be administered after bone marrow transplants and peripheral blood stem cell transplantation. Bone marrow transplantation and peripheral blood stem cell transplantation are procedures that restore stem cells that were destroyed by high doses of chemotherapy and/or radiation therapy. After being treated with high-dose anticancer drugs and/or radiation, the patient receives harvested stem cells, which travel to the bone marrow and begin to produce new blood cells. A “mini-transplant” uses lower, less toxic doses of chemotherapy and/or radiation to prepare the patient for transplant. A “tandem transplant” involves two sequential courses of high-dose chemotherapy and stem cell transplant. In autologous transplants, patients receive their own stem cells. In syngeneic transplants, patients receive stem cells from their identical twin. In allogeneic transplants, patients receive stem cells from their brother, sister, or parent. A person who is not related to the patient (an unrelated donor) also may be used. In some types of leukemia, the graft-versus-tumor (GVT) effect that occurs after allogeneic BMT and PBSCT is crucial to the effectiveness of the treatment. GVT occurs when white blood cells from the donor (the graft) identify the cancer cells that remain in the patient's body after the chemotherapy and/or radiation therapy (the tumor) as foreign and attack them. Immunotherapy with the combination therapy described herein can take advantage of this by vaccinating after a transplant. Additionally, the transferred cells may be presented with neoantigens of the combination therapy described herein before transplantation.

In one embodiment the combination therapy is administered to a patient in need thereof with a cancer that requires surgery. In one embodiment the combination therapy described herein is administered to a patient in need thereof in a cancer where the standard of care is primarily surgery followed by treatment to remove possible micro-metastases, such as breast cancer. Breast cancer is commonly treated by various combinations of surgery, radiation therapy, chemotherapy, and hormone therapy based on the stage and grade of the cancer. Adjuvant therapy for breast cancer is any treatment given after primary therapy to increase the chance of long-term survival. Neoadjuvant therapy is treatment given before primary therapy. Adjuvant therapy for breast cancer is any treatment given after primary therapy to increase the chance of long-term disease-free survival. Primary therapy is the main treatment used to reduce or eliminate the cancer. Primary therapy for breast cancer usually includes surgery, a mastectomy (removal of the breast) or a lumpectomy (surgery to remove the tumor and a small amount of normal tissue around it; a type of breast-conserving surgery). During either type of surgery, one or more nearby lymph nodes are also removed to see if cancer cells have spread to the lymphatic system. When a woman has breast-conserving surgery, primary therapy almost always includes radiation therapy. Even in early-stage breast cancer, cells may break away from the primary tumor and spread to other parts of the body (metastasize). Therefore, doctors give adjuvant therapy to kill any cancer cells that may have spread, even if they cannot be detected by imaging or laboratory tests.

In one embodiment the combination therapy is administered consistent with the standard of care for Ductal carcinoma in situ (DCIS). The standard of care for this breast cancer type is:

1. Breast-conserving surgery and radiation therapy with or without tamoxifen.
2. Total mastectomy with or without tamoxifen.
3. Breast-conserving surgery without radiation therapy.

The combination therapy may be administered before breast conserving surgery or total mastectomy to shrink the tumor before surgery.

In another embodiment the combination therapy can be administered as an adjuvant therapy to remove any remaining cancer cells.

In another embodiment patients diagnosed with stage I, II, IIIA, and Operable IIIC breast cancer are treated with the combination therapy as described herein. The standard of care for this breast cancer type is:
1. Local-regional treatment:
Breast-conserving therapy (lumpectomy, breast radiation, and surgical staging of the axilla).
Modified radical mastectomy (removal of the entire breast with level I-II axillary dissection) with or without breast reconstruction.
Sentinel node biopsy.
   2. Adjuvant radiation therapy postmastectomy in axillary node-positive tumors:
For one to three nodes: unclear role for regional radiation (infra/supraclavicular nodes, internal mammary nodes, axillary nodes, and chest wall).
For more than four nodes or extranodal involvement: regional radiation is advised.
   3. Adjuvant systemic therapy

In one embodiment the combination therapy is administered as a neoadjuvant therapy to shrink the tumor. In another embodiment the combination is administered as an adjuvant systemic therapy.

In another embodiment patients diagnosed with inoperable stage IIIB or IIIC or inflammatory breast cancer are treated with the combination therapy as described herein. The standard of care for this breast cancer type is:

1. Multimodality therapy delivered with curative intent is the standard of care for patients with clinical stage IIIB disease.
2. Initial surgery is generally limited to biopsy to permit the determination of histology, estrogen-receptor (ER) and progesterone-receptor (PR) levels, and human epidermal growth factor receptor 2 (HER2/neu) overexpression. Initial treatment with anthracycline-based chemotherapy and/or taxane-based therapy is standard. For patients who respond to neoadjuvant chemotherapy, local therapy may consist of total mastectomy with axillary lymph node dissection followed by postoperative radiation therapy to the chest wall and regional lymphatics. Breast-conserving therapy can be considered in patients with a good partial or complete response to neoadjuvant chemotherapy. Subsequent systemic therapy may consist of further chemotherapy. Hormone therapy should be administered to patients whose tumors are ER-positive or unknown. All patients should be considered candidates for clinical trials to evaluate the most appropriate fashion in which to administer the various components of multimodality regimens.

In one embodiment the combination therapy is administered as part of the various components of multimodality regimens. In another embodiment the combination therapy is administered before, simultaneously with, or after the multimodality regimens. In another embodiment the combination therapy is administered based on synergism between the modalities. In another embodiment the combination therapy is administered after treatment with anthracycline-based chemotherapy and/or taxane-based therapy (Zitvogel et al., 2008). Treatment after administering the combination therapy may negatively affect dividing effector T-cells. The combination therapy may also be administered after radiation.

In another embodiment the combination therapy described herein is used in the treatment in a cancer where the standard of care is primarily not surgery and is primarily based on systemic treatments, such as Chronic Lymphocytic Leukemia (CLL).

In another embodiment patients diagnosed with stage I, II, III, and IV Chronic Lymphocytic Leukemia are treated with the combination therapy as described herein. The standard of care for this cancer type is:

1. Observation in asymptomatic or minimally affected patients

### 2. Rituximab

### 3. Ofatumomab

4. Oral alkylating agents with or without corticosteroids
5. Fludarabine, 2-chlorodeoxyadenosine, or pentostatin

### 6. Bendamustine

### 7. Lenalidomide

8. Combination chemotherapy.
combination chemotherapy regimens include the following:
Fludarabine plus cyclophosphamide plus rituximab.
Fludarabine plus rituximab as seen in the CLB-9712 and CLB-9011 trials.
Fludarabine plus cyclophosphamide versus fludarabine plus cyclophosphamide plus rituximab.
Pentostatin plus cyclophosphamide plus rituximab as seen in the MAYO-MC0183 trial, for example.
Ofatumumab plus fludarabine plus cyclophosphamide.
CVP: cyclophosphamide plus vincristine plus prednisone.
CHOP: cyclophosphamide plus doxorubicin plus vincristine plus prednisone.
Fludarabine plus cyclophosphamide versus fludarabine as seen in the E2997 trial [NCT00003764] and the LRF-CLL4 trial, for example.
Fludarabine plus chlorambucil as seen in the CLB-9011 trial, for example.
   9. Involved-field radiation therapy.

### 10. Alemtuzumab

11. Bone marrow and peripheral stem cell transplantations are under clinical evaluation.

### 12. Ibrutinib

In one embodiment the combination therapy is administered before, simultaneously with or after treatment with Rituximab or Ofatumomab. As these are monoclonal antibodies that target B-cells, treatment with the combination therapy may be synergistic. In another embodiment the combination therapy is administered after treatment with oral alkylating agents with or without corticosteroids, and Fludarabine, 2-chlorodeoxyadenosine, or pentostatin, as these treatments may negatively affect the immune system if administered before. In one embodiment bendamustine is administered with the combination therapy in low doses based on the results for prostate cancer described herein. In one embodiment the combination therapy is administered after treatment with bendamustine.

In another embodiment, therapies targeted to specific recurrent mutations in genes that include extracellular domains are used in the treatment of a patient in need thereof suffering from cancer. The genes may advantageously be well-expressed genes. Well expressed may be expressed in “transcripts per million” (TPM). A TPM greater than 100 is considered well expressed. Well expressed genes may be FGFR3, ERBB3, EGFR, MUC4, PDGFRA, MMP12, TMEM52, and PODXL. The therapies may be a ligand capable of binding to an extracellular neoantigen epitope. Such ligands are well known in the art and may include therapeutic antibodies or fragments thereof, antibody-drug conjugates, engineered T cells, or aptamers. Engineered T cells may be chimeric antigen receptors (CARs). Antibodies may be fully humanized, humanized, or chimeric. The antibody fragments may be a nanobody, Fab, Fab′, (Fab′)2, Fv, ScFv, diabody, triabody, tetrabody, Bis-scFv, minibody, Fab2, or Fab3 fragment. Antibodies may be developed against tumor-specific neoepitopes using known methods in the art.

### Computer Systems and Implemented Methods

The invention also provides a computer memory system or a programmable or data-manipulating computer or data-manipulating memory or data-manipulating computer memory system comprising a library of neoantigen molecule information from any of the methods described herein or from the library of molecules described herein, or a library of neoantigen molecule information from any of the methods described herein or from the library of molecules described herein, contained within a computer memory system or a programmable or data-manipulating computer or data-manipulating memory or data-manipulating computer memory system.

The invention provides a method for determining or preparing a neoantigen composition comprising transcriptional analysis of subject-specific genetic information and/or whole genome or whole exome sequencing analysis of subject-specific genetic information (individually and collectively “transcriptional analysis”) and comparing results of that analysis with the library described herein or inputting information from the system described herein into a database having the transcriptional analysis of subject-specific genetic information or inputting information from the transcriptional analysis of subject-specific genetic information into the system described herein, and ascertaining those neoantigens that are common to the library or system and the transcriptional analysis of subject-specific genetic information.

In some embodiments, the method is for ranking those neoantigens to be included in a pharmaceutical composition, and those neoantigens common to the library or system and the transcriptional analysis of subject-specific genetic information are ranked highly for including in the pharmaceutical composition.

In some embodiments, the method comprises admixing into a pharmaceutical preparation neoantigens ascertained to be common or ranked highly for including in the pharmaceutical composition; optionally also including synthesizing one or more of the neoantigens.

The invention provides a method of determining or preparing a shared neoantigen comprising the library described herein or information from the system described herein comprising more than one set of neoantigens or neoantigen molecular information, or a statistically significant number of sets of neoantigens or neoantigen molecular information, and (a) comparing the sets for neoantigens having identical sequences and selecting those neoantigens having identical sequences as shared neoantigens, and/or (b) comparing sets for neoantigens having any of at least 99%, 98%, 97%, 96%, 95%, 94%, 93%, 92%, 91%, 90%, 89%, 88%, 87%, 86%, 85%, 84%, 83%, 82%, 81%, or 80% identical sequences for shared neoantigens or candidate shared neoantigens; optionally if (b) and if the comparing is for candidate shared neoantigens, further comprising selecting a neoantigen consensus sequence for the shared neoantigen, optionally wherein the selecting comprises selecting for the shared neoantigen sequence aligned areas or areas of same amino acids of the percent identical sequences and where not aligned or same amino acids selecting amino acids that positionally most occur across the sets and/or selecting based positional commonality of molecular size and charge of amino acids occurring across the sets.

In some embodiments, the method comprises comparing transcriptional analysis information as to the sets of (a) and the shared neoantigen composition comprising those neoantigens having identical sequences that are common to the library described herein or information from the system described herein and the transcriptional analysis information; or including comparing transcriptional analysis information as to the sets of (b) and the shared neoantigen composition comprising those neoantigens having the percent identical sequence library described herein or information from the system described herein and the transcriptional analysis information; optionally if (b) and if the comparing is for candidate shared neoantigens, further comprising selecting a neoantigen consensus sequence for the shared neoantigen, optionally wherein the selecting comprises selecting for the shared neoantigen sequence aligned areas or areas of same amino acids of the percent identical sequences and where not aligned or same amino acids selecting amino acids that positionally most occur across the sets and/or selecting based positional commonality of molecular size and charge of amino acids occurring across the sets.

In some embodiments, the method comprises admixing into a pharmaceutical preparation shared neoantigens ascertained by the method; optionally also including synthesizing one or more of the shared neoantigens.

The invention provides a method for determining or preparing a neopolypeptide composition comprising transcriptional analysis of subject-specific genetic information and/or whole genome or whole exome sequencing analysis of subject-specific genetic information (individually and collectively “transcriptional analysis”) and comparing results of that analysis with the library described herein or inputting information from the system described herein into a database having the transcriptional analysis of subject-specific genetic information or inputting information from the transcriptional analysis of subject-specific genetic information into the system described herein, and ascertaining those neopolypeptides that are common to the library or system and the transcriptional analysis of subject-specific genetic information.

In some embodiments, the method for determining or preparing a neopolypeptide composition is for ranking those neopolypeptides to be included in the composition, and those neopolypeptides common to the library or system and the transcriptional analysis of subject-specific genetic information are ranked highly for including in the composition. In some embodiments, the method comprises admixing into a pharmaceutical preparation neopolypeptides ascertained to be common or ranked highly for including in the pharmaceutical composition; optionally also including synthesizing one or more of the neopolypeptides.

The invention provides a method of determining or preparing a neopolypeptide common across subjects having the genetic disorder (shared neopolypeptides) comprising the library described herein or information from the system described herein comprising more than one set of neopolypeptides or neopolypeptide molecular information, or a statistically significant number of sets of neopolypeptides or neopolypeptide molecular information, and (a) comparing the sets for neopolypeptides having identical sequences and selecting those neopolypeptides having identical sequences as shared neopolypeptides, and/or (b) comparing sets for neopolypeptides having any of at least 99%, 98%, 97%, 96%, 95%, 94%, 93%, 92%, 91%, 90%, 89%, 88%, 87%, 86%, 85%, 84%, 83%, 82%, 81%, or 80% identical sequences for shared neopolypeptides or candidate shared neopolypeptides; optionally if (b) and if the comparing is for candidate shared neopolypeptides, further comprising selecting a neopolypeptide consensus sequence for the shared neopolypeptide, optionally wherein the selecting comprises selecting for the shared neopolypeptide sequence aligned areas or areas of same amino acids of the percent identical sequences and where not aligned or same amino acids selecting amino acids that positionally most occur across the sets and/or selecting based positional commonality of molecular size and charge of amino acids occurring across the sets.

In some embodiments, the method comprises comparing transcriptional analysis information as to the sets of (a) and the shared neopolypeptide composition comprising those neopolypeptides having identical sequences that are common to the library described herein or information from the system described herein and the transcriptional analysis information; or including comparing transcriptional analysis information as to the sets of (b) and the shared neopolypeptide composition comprising those neopolypeptides having the percent identical sequence library described herein or information from the system described herein and the transcriptional analysis information; optionally if (b) and if the comparing is for candidate shared neopolypeptides, further comprising selecting a neopolypeptide consensus sequence for the shared neopolypeptide, optionally wherein the selecting comprises selecting for the shared neopolypeptide sequence aligned areas or areas of same amino acids of the percent identical sequences and where not aligned or same amino acids selecting amino acids that positionally most occur across the sets and/or selecting based positional commonality of molecular size and charge of amino acids occurring across the sets. In some embodiments, the method comprises admixing into a preparation shared neopolypeptides ascertained by the method; optionally also including synthesizing one or more of the shared neopolypeptides.

### Screening

The invention provides a method for screening a eukaryotic or mammalian or human cell sample for whether the sample may have a genetic disorder comprising analyzing the cell sample for cell(s) having expression of neopolypeptide(s) comprised within the library described herein or information from the system described herein.

The invention provides a method for screening a eukaryotic or mammalian or human cell sample for whether the sample may have a pathogenic disorder comprising analyzing the cell sample for cell(s) having expression of neopolypeptide(s) comprised within the library described herein or information from the system described herein.

The invention provides a method of determining or screening for or preparing a treatment or modality for addressing a pathogenic disorder or a condition or symptom of a pathogenic disorder comprising perturbing a non-pathogenic disorder eukaryotic or mammalian or human cell by mutating the cell so as to have cell(s) having mutation(s) whereby the expression of the cell(s) comprise(s) neopolypeptide(s) comprised within the library described herein or information from the system described herein; optionally contacting the cell(s) with putative agent(s) to upregulate or downregulate phenotypic difference(s) between the cell(s) and a non-perturbed cell; optionally including detecting phenotypic difference(s) between the cell(s) and a non-perturbed cell; optionally further including detecting whether the contacting so upregulates or downregulates the phenotypic difference(s).

In some embodiments, the mutating the cell comprises contacting the cell with an engineered zinc finger or TALENs or CRISPR system that induces the mutation(s).

The invention provides an engineered zinc finger or TALENs or CRISPR system that modifies a eukaryotic or mammalian or human cell so that the cell has mutation(s) whereby cell expression comprises neopolypeptide(s) comprised within the library described herein or information from the system described herein.

The invention provides a CRISPR system that modifies a eukaryotic or mammalian or human cell so that the cell to has mutation(s) whereby cell expression comprises neopolypeptide(s) comprised within the library described herein or information from the system described herein.

In some embodiments, the CRISPR system comprises a CRISPR-Cas9 or CRISPR-Cas12a or CRISPR-Cpf1 system.

In some embodiments, the CRISPR system comprises guide(s) that target pathogenic locus or loci that comprises coding to be modified, whereby when modified by the CRISPR system the cell has the mutation(s).

In some embodiments, the CRISPR system comprises guides that target pathogenic locus or loci that comprises coding to be modified, whereby when modified by the CRISPR system the cell has the mutations.

In some embodiments, modification(s) by the CRISPR system comprise(s) insertion, deletion, or substitution of one or more nucleotides to give rise to the cell having the mutation(s).

The invention provides a method for perturbing a eukaryotic or mammalian or human cell so as to alter phenotype including so that the cell or progeny thereof express neopolypeptide(s) comprised within the library described herein or information from the system described herein comprising contacting the cell with a zinc finger or TALENs or CRISPR system.

### T Cells Specific to Neoantigens

In certain embodiments, T cells are obtained that are specific for any peptides identified according to the methods described herein or disclosed herein. The T cells may be obtained by screening a population of T cells with identified peptides according to US20180000913A1 which is the U.S. National Phase Application of International Patent Application No. PCT/US2015/067154. The T cells may be obtained from a patient. The T cells may be obtained from PBMCs obtained from a blood sample of the patient. The T cells may be identified by binding to reporter cells expressing a neoantigen and a reporter gene. The T cells may be identified by binding tetramers loaded with neoantigens to the T cells. The tetramers may be fluorescently labeled. The T cells bound by labeled tetramers may be isolated by FACS. The isolated T cells may be activated and expanded. In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. In a preferred embodiment, neoantigens are used.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

In certain embodiments, T cells are generated by using a boost/prime method (see, e.g., poster 197 SITC 2019—Society for Immunotherapy of Cancer; and neontherapeutics.com/wp-content/uploads/PTC-01_SITC2019_191104.pdf. Briefly, methods of administering an initial vector, for example an AV vector, comprising one or more neoantigens to generate an initial T cell response followed by a subsequent administration of a vector, for example, a samRNA vector, that can drive an antigen-specific T cell response can be utilized. Administration of either can be administered alone, together, or at varying timepoints.

### Adoptive Cell Transfer (ACT)

In certain embodiments, T cells specific for neoantigens as described herein are used in the treatment of cancer. Methods of identifying subject-specific T cell receptor (TCR) pairs suitable for subject-specific cancer therapy are also provided and may comprise: isolating from the subject a population comprising T cells; determining by single cell sequencing the sequences encoding the TCR pairs on individual cells in the population isolated; transfecting or transducing T cell lines deficient in endogenous TCRs with the sequences encoding individual TCR pairs determined; and using the T cell lines to assay binding of the subject specific TCR pairs to subject specific neoepitopes and selecting the TCR pairs that bind to subject-specific neoepitopes. In certain methods, the subject specific neoepitopes are expressed on HLA molecules on a cell. Cells may be antigen presenting cells. Binding of the T cells to the neoepitopes may activate a reporter gene, and neoepitopes may be present in tetramers. In an aspect, the neoepitopes are nuORFs.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 Jun.; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of any neoantigen identified according to the methods described herein or any neoantigen described herein.

Various strategies may for example be employed to genetically modify T cells by altering the specificity of the T cell receptor (TCR) for example by introducing new TCR a and chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIM, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ. I.D. No. 3). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signalling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am.J.Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CART cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 Oct.; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CART cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In an aspect, the composition further comprising at least one modulator of a checkpoint molecule or an immunomodulator, or a nucleic acid encoding the modulator or immunomodulator, or a vector comprises the nucleic acid encoding the modulator or immunomodulator for use in preventing or treating a proliferative disease in a subject, which may be an agonist of a tumor necrosis factor receptor superfamily member, preferably of CD27, CD40, OX40, GITR, or CD137; and/or an antagonist of PD-1, PD-L1, CD274, A2AR, B7-H3, B7-H4, BTLA<CTLA-4, IDO, KIR, LAG3, TIM-3, VISTA, or an antagonist of a B7-CD28 superfamily member, preferably of CD28 or ICOS or an antagonist of a ligand thereof; and/or the immunomodulator is a T cell growth factor, preferably IL-2, IL-12, or IL-15.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

Selecting the Patient Population Most Likely to Benefit from the Therapy

In another aspect, the invention provides selecting for the patients in need thereof most likely to benefit from the therapy of the present invention. Although the compositions and methods of the present invention are typically applicable in a high proportion of subjects suffering from cancer, the method may still comprise one or more steps of selecting patients from the patient population who are likely to benefit. For instance, the method may comprise selecting subjects whose tumors contain one or more of the mutations represented in the neoantigenic peptides in the composition. In another embodiment, the method may comprise selecting subjects having at least one HLA allele which binds to one or more neoepitopes represented in the neoantigenic peptides in the composition.

Methods for determining or preparing a neopolypeptide composition may comprise transcriptional analysis of subject-specific pathogenic information and/or whole genome or whole exome sequencing analysis of subject-specific pathogenic information (individually and collectively “transcriptional analysis.” This analysis may further comprise comparing results of that analysis with a library generated from ribosomal translational analysis or any of the analyses or library generation methods disclosed elsewhere herein. Information may be input from the computer systems disclosed and ascertaining neopolypeptides that are common to the library or system and the transcriptional analysis of subject specific pathogenic information. This information can aid in selecting treatments and/or identifying patient populations most likely to benefit from the therapy. In these and methods described throughout the application, a variety of sequencing approaches and perturbations may be utilized.

Accordingly, the methods may be utilized for screening applications. The invention provides a method of determining or screening for or preparing a treatment or modality for addressing a cancer or a condition or symptom of a cancer comprising perturbing a non-cancer eukaryotic or mammalian or human cell by mutating the cell so as to have cell(s) having mutation(s) whereby the expression of the cell(s) comprise(s) neoantigen(s) comprised within the library described herein or information from the system described herein; optionally contacting the cell(s) with putative agent(s) to upregulate or downregulate phenotypic difference(s) between the cell(s) and a non-perturbed cell; optionally including detecting phenotypic difference(s) between the cell(s) and a non-perturbed cell; optionally further including detecting whether the contacting so upregulates or downregulates the phenotypic difference(s).

In some embodiments, the mutating the cell comprises contacting the cell with an engineered zinc finger or TALENs or CRISPR system that induces the mutation(s).

The invention provides an engineered zinc finger or TALENs or CRISPR system that modifies a eukaryotic or mammalian or human cell so that the cell has mutation(s) whereby cell expression comprises neoantigen(s) comprised within the library described herein or information from the system described herein.

The invention provides a CRISPR system that modifies a eukaryotic or mammalian or human cell so that the cell to has mutation(s) whereby cell expression comprises neoantigen(s) comprised within the library described herein or information from the system described herein.

In some embodiments, the CRISPR system comprises a CRISPR-Cas9 or CRISPR-Cas12a or CRISPR-Cpf1 system.

In some embodiments, the CRISPR system comprises guide(s) that target genetic locus or loci that comprises coding to be modified, whereby when modified by the CRISPR system the cell has the mutation(s).

In some embodiments, the CRISPR system comprises guides that target genetic locus or loci that comprises coding to be modified, whereby when modified by the CRISPR system the cell has the mutations.

In some embodiments, modification(s) by the CRISPR system comprise(s) insertion, deletion, or substitution of one or more nucleotides to give rise to the cell having the mutation(s).

The invention provides a method for perturbing a eukaryotic or mammalian or human cell so as to alter phenotype including so that the cell or progeny thereof express neoantigen(s) comprised within the library described herein or information from the system described herein comprising contacting the cell with any one of the zinc finger or Talens or CRISPR systems described herein.

### MS Methods

Detection may also be evaluated using mass spectrometry methods. Immunopeptidome data used in methods disclosed herein may also be mass spectrometry data, in an aspect the data is MS/MS data. A variety of configurations of mass spectrometers can be used. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein values, including immunppeptidome data, can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Cite Seq

Protocols for Cellular Indexing of Transcriptome and Epitopes by sequencing (CITE-seq) are set forth in Stoeckius M, et al., Simultaneous epitope and transcriptome measurement in single cells 31 Jul. 2017, Nature Methods 9, 2579-10 (2017) and Stoeckius, M. & Smibert, Cite-seq, Protocol Exchange doi.org/10.1038/protex.2017.068 (31 Jul. 2017). Reference is made to US Patent Publication 20180251825, in particular FIGS. 1A-1C providing a schematic of CITE-seq which allows for simultaneous measurement of large numbers of established antibody-based markers along with unbiased single-cell transcriptome data, on the scale of tens of thousands of cells per experiment. The inventors of US Patent Publication 20180251825 devised a digital, sequencing-based readout for protein levels by conjugating antibodies to oligonucleotides (oligos) that can be captured by oligo-dT primers (used in most scRNA-seq library preparations), contain a barcode for antibody identification and include a handle for PCR amplification. A commonly used streptavidin-biotin interaction links the 5′ end of oligos to antibodies. The antibody-oligo complexes are incubated with single-cell suspensions in conditions comparable to flow cytometry staining protocols; after this incubation, cells are washed to remove unbound antibodies and processed for scRNA-seq. The inventors of US Patent Publication 20180251825 encapsulated single cells into nanolitersized aqueous droplets in a microfluidic apparatus designed to perform Drop-seq. After cell lysis in droplets, cellular mRNAs and antibody-derived oligos both anneal via their 3′ polyA tails to Drop-seq beads containing oligo-dT and are indexed by a shared cellular barcode during reverse transcription. The amplified cDNAs and antibody-derived tags (ADTs) can be separated by size and converted into Illumina-sequencing libraries independently. Importantly, because the two library types are generated separately, their relative proportions can be adjusted in a pooled single lane to ensure that the required sequencing depth is obtained for each library.

### Perturb Seq

Perturb-seq (also known as CRISP-seq and CROP-seq) refers to a high-throughput method of performing single cell RNA sequencing (scRNA-seq) on pooled genetic perturbation screens (see, e.g., Cell. 167 (7): 1867-1882.e21. doi:10.1016/j.cell.2016.11.048; Cell. 167 (7): 1853-1866.e17. doi:10.1016/j.cell.2016.11.038; Nature Methods. 14 (3): 297-301. doi:10.1038/nmeth.417 and international patent publication WO 2017/075294). Perturb-seq combines multiplexed CRISPR mediated gene inactivations with single cell RNA sequencing to assess comprehensive gene expression phenotypes for each perturbation. Inferring a gene's function by applying genetic perturbations to knock down or knock out a gene and studying the resulting phenotype is known as reverse genetics. Perturb-seq is a reverse genetics approach that allows for the investigation of phenotypes at the level of the transcriptome, to elucidate gene functions in many cells, in a massively parallel fashion. The Perturb-seq protocol uses CRISPR technology to inactivate specific genes and DNA barcoding of each guide RNA to allow for all perturbations to be pooled together and later deconvoluted, with assignment of each phenotype to a specific guide RNA. Droplet-based microfluidics platforms (or other cell sorting and separating techniques) are used to isolate individual cells, and then scRNA-seq is performed to generate gene expression profiles for each cell. Upon completion of the protocol, bioinformatics analyses are conducted to associate each specific cell and perturbation with a transcriptomic profile that characterizes the consequences of inactivating each gene.

Perturb-seq, which combines single cell RNA-seq and CRISPR/Cas9 based perturbations identified by unique polyadenylated barcodes to perform many, tens of thousands in certain embodiments, of such assays in a single pooled experiment. By randomly integrating more than one sgRNA in each cell, Perturb-Seq is extended to test transcriptional phenotypes caused by genetic interactions. Applicants develop a computational framework, MIMOSCA (Multi-Input Multi-Output Single Cell Analysis) to identify the regulatory effects of individual perturbations and their combinations at different levels of resolution: from effects on each individual gene to functional signatures to proportional changes in cell types. Applicants demonstrate Perturb-seq by analyzing 200,000 cells across three screens: transcription factors controlling the immune response of dendritic cells to LPS, transcription factors bound in the K562 cell line, and cell cycle regulators in the same cell line. Perturb-Seq accurately identified known regulatory relations, and its individual gene target predictions were validated by ChIP-Seq binding profiles. Applicants posit new functions for regulatory factors affecting cell differentiation, the anti-viral response and mitochondrial function during immune activation, and uncovered an underlying circuit that balances these different programs through positive and negative feedback loops. Using Perturb-Seq Applicants identified genetic interactions including synergistic, buffering and dominant genetic interactions that could not be predicted from individual perturbations alone. Perturb-Seq can be flexibly applied to diverse cell metadata, to customize design and scope of pooled genomic assays.

In one aspect, the present invention provides for a method of reconstructing a cellular network or circuit, comprising introducing at least 1, 2, 3, 4 or more single-order or combinatorial perturbations to a plurality of cells in a population of cells, wherein each cell in the plurality of the cells receives at least 1 perturbation; measuring comprising: detecting genomic, genetic, proteomic, epigenetic and/or phenotypic differences in single cells compared to one or more cells that did not receive any perturbation, and detecting the perturbation(s) in single cells; and determining measured differences relevant to the perturbations by applying a model accounting for co-variates to the measured differences, whereby intercellular and/or intracellular networks or circuits are inferred. The measuring in single cells may comprise single cell sequencing. The single cell sequencing may comprise cell barcodes, whereby the cell-of-origin of each RNA is recorded. The single cell sequencing may comprise unique molecular identifiers (UMI), whereby the capture rate of the measured signals, such as transcript copy number or probe binding events, in a single cell is determined. The model may comprise accounting for the capture rate of measured signals, whether the perturbation actually perturbed the cell (phenotypic impact), the presence of subpopulations of either different cells or cell states, and/or analysis of matched cells without any perturbation.

The single-order or combinatorial perturbations may comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 perturbations. The perturbation(s) may target genes in a pathway or intracellular network. [0027] The measuring may comprise detecting the transcriptome of each of the single cells. The perturbation(s) may comprise one or more genetic perturbation(s). The perturbation(s) may comprise one or more epigenetic or epigenomic perturbation(s). At least one perturbation may be introduced with RNAi- or a CRISPR-Cas system. At least one perturbation may be introduced via a chemical agent, biological agent, an intracellular spatial relationship between two or more cells, an increase or decrease of temperature, addition or subtraction of energy, electromagnetic energy, or ultrasound.

The cell(s) may comprise a cell in a model non-human organism, a model non-human mammal that expresses a Cas protein, a mouse that expresses a Cas protein, a mouse that expresses Cpf1, a cell in vivo or a cell ex vivo or a cell in vitro. The cell(s) may also comprise a human cell.

The measuring or measured differences may comprise measuring or measured differences of DNA, RNA, protein or post translational modification; or measuring or measured differences of protein or post translational modification correlated to RNA and/or DNA level(s).

The perturbing or perturbation(s) may comprise(s) genetic perturbing. The perturbing or perturbation(s) may comprise(s) single-order perturbations. The perturbing or perturbation(s) may comprise(s) combinatorial perturbations. The perturbing or perturbation(s) may comprise gene knock-down, gene knock-out, gene activation, gene insertion, or regulatory element deletion. The perturbing or perturbation(s) may comprise genome-wide perturbation. The perturbing or perturbation(s) may comprise performing CRISPR-Cas-based perturbation. The perturbing or perturbation(s) may comprise performing pooled single or combinatorial CRISPR-Cas-based perturbation with a genome-wide library of sgRNAs. The perturbations may be of a selected group of targets based on similar pathways or network of targets.

The perturbing or perturbation(s) may comprises performing pooled combinatorial CRISPR-Cas-based perturbation with a genome-wide library of sgRNAs. Each sgRNA may be associated with a unique perturbation barcode. Each sgRNA may be co-delivered with a reporter mRNA comprising the unique perturbation barcode (or sgRNA perturbation barcode).

The perturbing or perturbation(s) may comprise subjecting the cell to an increase or decrease in temperature. The perturbing or perturbation(s) may comprise subjecting the cell to a chemical agent. The perturbing or perturbation(s) may comprise subjecting the cell to a biological agent. The biological agent may be a toll like receptor agonist or cytokine. The perturbing or perturbation(s) may comprise subjecting the cell to a chemical agent, biological agent and/or temperature increase or decrease across a gradient.

The cell may be in a microfluidic system. The cell may be in a droplet. The population of cells may be sequenced by using microfluidics to partition each individual cell into a droplet containing a unique barcode, thus allowing a cell barcode to be introduced.

The perturbing or perturbation(s) may comprise transforming or transducing the cell or a population that includes and from which the cell is isolated with one or more genomic sequence-perturbation constructs that perturbs a genomic sequence in the cell. The sequence-perturbation construct may be a viral vector, preferably a lentivirus vector. The perturbing or perturbation(s) may comprise multiplex transformation or transduction with a plurality of genomic sequence-perturbation constructs.

In another aspect, or in alternative embodiments of aspects described herein, the present invention provides for a method wherein proteins or transcripts expressed in single cells are determined in response to a perturbation, wherein the proteins or transcripts are detected in the single cells by binding of more than one labeling ligand comprising an oligonucleotide tag, and wherein the oligonucleotide tag comprises a unique constituent identifier (UCI) specific for a target protein or transcript. The single cells may be fixed in discrete particles. The discrete particles may be washed and sorted, such that cell barcodes may be added, e.g. sgRNA perturbation barcodes as described above. The oligonucleotide tag and sgRNA perturbation barcode may comprise a universal ligation handle sequence, whereby a unique cell barcode may be generated by split-pool ligation. The labeling ligand may comprise an oligonucleotide label comprising a regulatory sequence configured for amplification by T7 polymerase. The labeling ligands may comprise oligonucleotide sequences configured to hybridize to a transcript specific region. Not being bound by a theory, both proteins and RNAs may be detected after perturbation. The oligonucleotide label may further comprise a photocleavable linker. The oligonucleotide label may further comprise a restriction enzyme site between the labeling ligand and unique constituent identifier (UCI). The ligation handle may comprise a restriction site for producing an overhang complementary with a first index sequence overhang, and wherein the method further comprises digestion with a restriction enzyme. The ligation handle may comprise a nucleotide sequence complementary with a ligation primer sequence and wherein the overhang complementary with a first index sequence overhang is produced by hybridization of the ligation primer to the ligation handle. The method may further comprise quantitating the relative amount of UCI sequence associated with a first cell to the amount of the same UCI sequence associated with a second cell, whereby the relative differences of a cellular constituent between cell(s) are determined. The labeling ligand may comprise an antibody or an antibody fragment. The antibody fragment may be a nanobody, Fab, Fab′, (Fab′)2, Fv, ScFv, diabody, triabody, tetrabody, Bis-scFv, minibody, Fab2, or Fab3 fragment. The labeling ligand may comprise an aptamer. The labeling ligand may be a nucleotide sequence complementary to a target sequence.

Single cell sequencing may comprise whole transcriptome amplification.

The method in aspects of the invention may comprise comparing an RNA profile of the perturbed cell with any mutations in the cell to also correlate phenotypic or transcriptome profile and genotypic profile.

In another aspect, or in alternative embodiments of aspects described herein, the present invention provides for a method comprising determining genetic interactions by causing a set of P genetic perturbations in single cells of the population of cells, wherein the method comprises: determining, based upon random sampling, a subset of π genetic perturbations from the set of P genetic perturbations; performing said subset of π genetic perturbations in a population of cells; performing single-cell molecular profiling of the population of genetically perturbed cells; inferring, from the results and based upon the random sampling, single-cell molecular profiles for the set of P genetic perturbations in cells. The method may further comprises: from the results, determining genetic interactions. The method may further comprise: confirming genetic interactions determined with additional genetic manipulations.

The set of P genetic perturbations or said subset of π genetic perturbations may comprise single-order genetic perturbations. The set of P genetic perturbations or said subset of π genetic perturbations may comprise combinatorial genetic perturbations. The genetic perturbation may comprise gene knock-down, gene knock-out, gene activation, gene insertion, or regulatory element deletion. The set of P genetic perturbations or said subset of π genetic perturbations may comprise genome-wide perturbations. The set of P genetic perturbations or said subset of π genetic perturbations may comprise k-order combinations of single genetic perturbations, wherein k is an integer ranging from 2 to 15, and wherein the method comprises determining k-order genetic interactions. The set of P genetic perturbations may comprise combinatorial genetic perturbations, such as k-order combinations of single-order genetic perturbations, wherein k is an integer ranging from 2 to 15, and wherein the method comprises determining j-order genetic interactions, with j<k.

The method in aspects of this invention may comprise performing RNAi- or CRISPR-Cas-based perturbation. The method may comprise an array-format or pool-format perturbation. The method may comprise pooled single or combinatorial CRISPR-Cas-based perturbation with a genome-wide library of sgRNAs. The method may comprise pooled combinatorial CRISPR-Cas-based perturbation with a genome-wide library of sgRNAs.

The random sampling may comprise matrix completion, tensor completion, compressed sensing, or kernel learning. The random sampling may comprise matrix completion, tensor completion, or compressed sensing, and wherein π is of the order of log P.

The cell may comprise a eukaryotic cell. The eukaryotic cell may comprise a mammalian cell. The mammalian cell may comprise a human cell. The cell may be from a population comprising 10<2> to 10<8> cells and DNA or RNA or protein or post translational modification measurements or variables per cell comprise 50 or more.

The perturbation of the population of cells may be performed in vivo. The perturbation of the population of cells may be performed ex vivo and the population of cells may be adoptively transferred to a subject. The population of cells may comprise tumor cells. The method may comprise a lineage barcode associated with single cells, whereby the lineage or clonality of single cells may be determined.

The perturbing may be across a library of cells to thereby obtain RNA level and/or optionally protein level, whereby cell-to-cell circuit data at genomic or transcript or expression level is determined. The library of cells may comprise or is from a tissue sample. The tissue sample may comprise or is from a biopsy from a mammalian subject. The mammalian subject may comprise a human subject. The biopsy may be from a tumor. The method may further comprise reconstructing cell-to-cell circuits.

In another aspect, the present invention provides a method of reconstructing a cellular network or circuit, comprising introducing at least 1, 2, 3, 4 or more single-order or combinatorial perturbations to each cell in a population of cells; measuring genomic, genetic and/or phenotypic differences of each cell and coupling combinatorial peturbations with measured differences to infer intercellular and/or intracellular networks or circuits. The single-order or combinatorial perturbations can comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99 or massively parallel perturbations. The perturbation(s) can comprise one or more genetic perturbation. The perturbation(s) can comprise one or more epigenetic or epigenomic perturbation. The perturbation can be introduced with RNAi- or a CRISPR-Cas system. For example, reference is also made to Dahlman et al., Nature Biotechnology (2015) doi:10.1038/nbt.3390 Published online 5 Oct. 2015 to allow efficient orthogonal genetic and epigenetic manipulation. Dahlman et al., Nature Biotechnology (2015) doi:10.1038/nbt.3390 have developed a CRISPR-based method that uses catalytically active Cas9 and distinct single guide (sgRNA) constructs to knock out and activate different genes in the same cell. These sgRNAs, with 14- to 15-bp target sequences and MS2 binding loops, can activate gene expression using an active Streptococcus pyogenes Cas9 nuclease, without inducing double-stranded breaks. Dahlman et al., Nature Biotechnology (2015) doi:10.1038/nbt.3390 use these ‘dead RNAs’ to perform orthogonal gene knockout and transcriptional activation in human cells.

The at least one perturbation can be introduced via a chemical agent, an intracellular spatial relationship between two or more cells, an increase or decrease of temperature, addition or subtraction of energy, electromagnetic energy, or ultrasound. The cell can comprise a cell in a model non-human organism, a model non-human mammal that expresses a Cas protein, a mouse that expresses a Cas protein, a cell in vivo or a cell ex vivo or a cell in vitro. The measuring or measured differences can comprise measuring or measured differences of DNA, RNA, protein or post translational modification; or measuring or measured differences of protein or post translational modification correlated to RNA and/or DNA level(s). The method can include sequencing, and prior to sequencing: perturbing and isolating a single cell with at least one labeling ligand specific for binding at one or more target RNA transcripts, or isolating a single cell with at least one labeling ligand specific for binding at one or more target RNA transcripts and perturbing the cell; and/or lysing the cell under conditions wherein the labeling ligand binds to the target RNA transcript(s).

The method in aspects of this invention may also include, prior to sequencing perturbing and isolating a single cell with at least one labeling ligand specific for binding at one or more target RNA transcripts, or isolating a single cell with at least one labeling ligand specific for binding at one or more target RNA transcripts and perturbing the cell; and lysing the cell under conditions wherein the labeling ligand binds to the target RNA transcript(s). The perturbing and isolating a single cell may be with at least one labeling ligand specific for binding at one or more target RNA transcripts. The isolating a single cell may be with at least one labeling ligand specific for binding at one or more target RNA transcripts and perturbing the cell.

The perturbing of the present invention may involve genetic perturbing, single-order genetic perturbations or combinatorial genetic perturbations. The perturbing may also involve gene knock-down, gene knock-out, gene activation, gene insertion or regulatory element deletion. The perturbation may be genome-wide perturbation. The perturbation may be performed by RNAi- or CRISPR-Cas-based perturbation, performed by pooled single or combinatorial CRISPR-Cas-based perturbation with a genome-wide library of sgRNAs or performing pooled combinatorial CRISPR-Cas-based perturbation with a genome-wide library of sgRNAs.

In another aspect, the methods described herein may be used for a diagnostic assay. In one embodiment, T cells are obtained from a subject and perturb-seq is performed on the cells. In another embodiment, T cells are obtained from a subject and gene expression of single cells is determined. Upon determining gene expression, perturb-seq is performed on a subset of genes differentially expressed. Perturb-seq can inform proper therapies to administer to a subject and can test many targets in a single experiment. In another embodiment, tumor cells are obtained from a subject. The tumor cells may also include cells of the tumor microenvironment, such as immune cells. The cells may be assayed for gene expression and differentially expressed genes can be assayed using the perturb-seq methods described herein. Not being bound by a theory, perturb-seq may allow assaying many targets and perturbations in a single experiment.

### Drop-Sequence Methods (“Drop-Seq”)

Cells, the basic units of biological structure and function, vary broadly in type and state. Single-cell genomics can characterize cell identity and function, but limitations of ease and scale have prevented its broad application. Macosko et al. (Cell. 2015 May 21; 161(5):1202-1214. doi: 10.1016/j.cell.2015.05.002) describe Drop-seq, a strategy for quickly profiling thousands of individual cells by separating them into nanoliter-sized aqueous droplets, associating a different barcode with each cell's RNAs, and sequencing them all together. Drop-seq analyzes mRNA transcripts from thousands of individual cells simultaneously while remembering transcripts' cell of origin. Macosko et al. analyzed transcriptomes from 44,808 mouse retinal cells and identified 39 transcriptionally distinct cell populations, creating a molecular atlas of gene expression for known retinal cell classes and novel candidate cell subtypes. Drop-seq accelerates biological discovery by enabling routine transcriptional profiling at single-cell resolution.

RNA profiling is in principle particularly informative, as cells express thousands of different RNAs. Approaches that measure for example the level of every type of RNA have until recently been applied to“homogenized” samples—in which the contents of all the cells are mixed together. Methods to profile the RNA content of tens and hundreds of thousands of individual human cells have been recently developed, including from brain tissues, quickly and inexpensively. To do so, special microfluidic devices have been developed to encapsulate each cell in an individual drop, associate the RNA of each cell with a ‘cell barcode’ unique to that cell/drop, measure the expression level of each RNA with sequencing, and then use the cell barcodes to determine which cell each RNA molecule came from.

Methods of Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; Zheng, et al., 2016, “Haplotyping germline and cancer genomes with high-throughput linked-read sequencing” Nature Biotechnology 34, 303-311; and International patent publication number WO 2014210353 A2 are contemplated for the present invention, all the contents and disclosure of each of which are herein incorporated by reference in their entirety.

Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets. The ability to conduct reactions in microdroplets depends on being able to merge different sample fluids and different microdroplets. See, e.g., US Patent Publication No. 20120219947. See also international patent application serial no. PCT/US2014/058637 for disclosure regarding a microfluidic laboratory on a chip.

Droplet microfluidics offers significant advantages for performing high-throughput screens and sensitive assays. Droplets allow sample volumes to be significantly reduced, leading to concomitant reductions in cost. Manipulation and measurement at kilohertz speeds enable up to 10<8> discrete biological entities (including, but not limited to, individual cells or organelles) to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

Drop-Sequence methods and apparatus provides a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read. A combination of molecular barcoding and emulsion-based microfluidics to isolate, lyse, barcode, and prepare nucleic acids from individual cells in high-throughput is used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode 10,000-100,000 cells.

The invention provides a method for creating a single-cell sequencing library comprising: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells; and preparing and sequencing a single composite RNA-Seq library.

The invention provides a method for preparing uniquely barcoded mRNA capture microbeads, which has a unique barcode and diameter suitable for microfluidic devices comprising: 1) performing reverse phosphoramidite synthesis on the surface of the bead in a pool-and-split fashion, such that in each cycle of synthesis the beads are split into four reactions with one of the four canonical nucleotides (T, C, G, or A) or unique oligonucleotides of length two or more bases; 2) repeating this process a large number of times, at least two, and optimally more than twelve, such that, in the latter, there are more than 16 million unique barcodes on the surface of each bead in the pool. (See www.ncbi.nlm.nih.gov/pmc/articles/PMC206447)

Generally, the invention provides a method for preparing a large number of beads, particles, microbeads, nanoparticles, or the like with unique nucleic acid barcodes comprising performing polynucleotide synthesis on the surface of the beads in a pool-and-split fashion such that in each cycle of synthesis the beads are split into subsets that are subjected to different chemical reactions; and then repeating this split-pool process in two or more cycles, to produce a combinatorially large number of distinct nucleic acid barcodes. Invention further provides performing a polynucleotide synthesis wherein the synthesis may be any type of synthesis known to one of skill in the art for“building” polynucleotide sequences in a step-wise fashion. Examples include, but are not limited to, reverse direction synthesis with phosphoramidite chemistry or forward direction synthesis with phosphoramidite chemistry. Previous and well-known methods synthesize the oligonucleotides separately then“glue” the entire desired sequence onto the bead enzymatically. Applicants present a complexed bead and a novel process for producing these beads where nucleotides are chemically built onto the bead material in a high-throughput manner. Moreover, Applicants generally describe delivering a “packet” of beads which allows one to deliver millions of sequences into separate compartments and then screen all at once.

The invention further provides an apparatus for creating a single-cell sequencing library via a microfluidic system, comprising: a oil-surfactant inlet comprising a filter and a carrier fluid channel, wherein said carrier fluid channel further comprises a resistor; an inlet for an analyte comprising a filter and a carrier fluid channel, wherein said carrier fluid channel further comprises a resistor; an inlet for mRNA capture microbeads and lysis reagent comprising a filter and a carrier fluid channel, wherein said carrier fluid channel further comprises a resistor; said carrier fluid channels have a carrier fluid flowing therein at an adjustable or predetermined flow rate; wherein each said carrier fluid channels merge at a junction; and said junction being connected to a mixer, which contains an outlet for drops.

A mixture comprising a plurality of microbeads adorned with combinations of the following elements: bead-specific oligonucleotide barcodes created by the described methods; additional oligonucleotide barcode sequences which vary among the oligonucleotides on an individual bead and can therefore be used to differentiate or help identify those individual oligonucleotide molecules; additional oligonucleotide sequences that create substrates for downstream molecular-biological reactions, such as oligo-dT (for reverse transcription of mature mRNAs), specific sequences (for capturing specific portions of the transcriptome, or priming for DNA polymerases and similar enzymes), or random sequences (for priming throughout the transcriptome or genome). In an embodiment, the individual oligonucleotide molecules on the surface of any individual microbead contain all three of these elements, and the third element includes both oligo-dT and a primer sequence.

Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added.

Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-di ethyl amino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAN/IRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

The fluorescent label may be a fluorescent protein, such as blue fluorescent protein, cyan fluorescent protein, green fluorescent protein, red fluorescent protein, yellow fluorescent protein or any photoconvertible protein. Colormetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code.

In an advantageous embodiment, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

The invention described herein enables high throughput and high resolution delivery of reagents to individual emulsion droplets that may contain cells, organelles, nucleic acids, proteins, etc. through the use of monodisperse aqueous droplets that are generated by a microfluidic device as a water-in-oil emulsion. The droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules. The aqueous droplets of volume ranging from 1 pL to 10 nL work as individual reactors. Disclosed embodiments provide 10<4> to 10<5> single cells in droplets which can be processed and analyzed in a single run.

To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination.

Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets.

Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. In another embodiment, the expansion and/or contraction of the liquid reservoir may be combined with other flow-controlling devices and methods, e.g., as described herein. Non-limiting examples of devices able to cause the expansion and/or contraction of a liquid reservoir include pistons.

Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets.

Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel.

Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform.

Droplets formed in oil without surfactant are not stable to permit coalescence, so surfactants must be dissolved in the oil that is used as the continuous phase for the emulsion library. Surfactant molecules are amphiphilic—part of the molecule is oil soluble, and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets.

A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification.

A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element.

A bead based library element may contain one or more beads, of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids. [00267] Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell.

Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges.

The droplets comprised within the emulsion libraries of the present invention may be contained within an immiscible oil which may comprise at least one fluorosurfactant. In some embodiments, the fluorosurfactant comprised within immiscible fluorocarbon oil is a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays described herein. Fluids (e.g., aqueous fluids, immiscible oils, etc.) and other surfactants that may be utilized in the droplet libraries of the present invention are described in greater detail herein.

The present invention provides an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library.

For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In one example, the cells may comprise cancer cells of a tissue biopsy, and each cell type is encapsulated to be screened for genomic data or against different drug therapies. Another example is that 10<11> or 10<15> different type of bacteria; each having a different plasmid spliced therein, are encapsulated. One example is a bacterial library where each library element grows into a clonal population that secretes a variant on an enzyme.

In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. In one specific example, a LacZ plasmid DNA was encapsulated at a concentration of 20 fM after two hours of incubation such that there was about one gene in 40 droplets, where 10 μm droplets were made at 10 kHz per second. Formation of these libraries rely on limiting dilutions. [00273] Methods of the invention involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc.

In certain embodiments, the carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing.

In certain embodiments, the droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates).

By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioninformatically record information can be found at US Provisional Patent Application entitled“Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional Patent Application entitled“Systems and Methods for Droplet Tagging” filed Sep. 21, 2012.

Applications of the disclosed device may include use for the dynamic generation of molecular barcodes (e.g., DNA oligonucleotides, flurophores, etc.) either independent from or in concert with the controlled delivery of various compounds of interest (drugs, small molecules, siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with cell-containing droplets. An electronic record in the form of a computer log file is kept to associate the barcode delivered with the downstream reagent(s) delivered. This methodology makes it possible to efficiently screen a large population of cells for applications such as single-cell drug screening, controlled perturbation of regulatory pathways, etc. The device and techniques of the disclosed invention facilitate efforts to perform studies that require data resolution at the single cell (or single molecule) level and in a cost effective manner. Disclosed embodiments provide a high throughput and high resolution delivery of reagents to individual emulsion droplets that may contain cells, nucleic acids, proteins, etc. through the use of monodisperse aqueous droplets that are generated one by one in a microfluidic chip as a water-in-oil emulsion. Hence, the invention proves advantageous over prior art systems by being able to dynamically track individual cells and droplet treatments/combinations during life cycle experiments. Additional advantages of the disclosed invention provides an ability to create a library of emulsion droplets on demand with the further capability of manipulating the droplets through the disclosed process(es). Disclosed embodiments may, thereby, provide dynamic tracking of the droplets and create a history of droplet deployment and application in a single cell based environment.

Droplet generation and deployment is produced via a dynamic indexing strategy and in a controlled fashion in accordance with disclosed embodiments of the present invention. Disclosed embodiments of the microfluidic device described herein provides the capability of microdroplets that be processed, analyzed and sorted at a highly efficient rate of several thousand droplets per second, providing a powerful platform which allows rapid screening of millions of distinct compounds, biological probes, proteins or cells either in cellular models of biological mechanisms of disease, or in biochemical, or pharmacological assays.

Single-Cell RNA Sequencing (scRNAseq)

Transcriptional profiling of thousands of single cells in parallel by RNA-seq is now routine. However, due to reliance on pooled library preparation, targeting analysis to particular cells of interest is difficult. Ranu et al. (Nucleic Acids Res. 2018 Sep. 26. doi: 10.1093/nar/gky856) present a multiplexed PCR method for targeted sequencing of select cells from pooled single-cell sequence libraries. Ranu et al. demonstrated this molecular enrichment method on multiple cell types within pooled single-cell RNA-seq libraries produced from primary human blood cells. Ranu et al. show how molecular enrichment can be combined with FACS to efficiently target ultra-rare cell types, such as the recently identified AXL+SIGLEC6+ dendritic cell (AS DC) subset, in order to reduce the required sequencing effort to profile single cells by 100-fold. Ranu et al.'s results demonstrate that DNA barcodes identifying cells within pooled sequencing libraries can be used as targets to enrich for specific molecules of interest, for example reads from a set of target cells.

Single-cell library preparation and target cell enrichment. Single-cell RNA-seq library preparation was performed with the Chromium Single Cell 3′ method (10× Genomics) according to the manufacturer's protocol. Pooled single-cell RNA-seq libraries were diluted and combined in equal volume with KAPA 2× high fidelity hot start PCR master mix. The final DNA template and total primer concentrations were 0.1 nM and 0.1 uM, respectively. For multiplex (10-15-plex) barcode amplification, forward primers consisted of sequencing adapters (62 bp) and cell barcode specific sequence (16 base pairs) whereas reverse primers were complimentary to the fixed truseq adaptor sequence. Hemi-specific PCR was performed with an initial hot start at 95° C. for 5 min, followed by 25 cycles of (95° C.-0.5 min, 68° C.-1 min, 72° C.-1 min), and ended with a final 4 min extension at 72° C. The reaction products were confirmed on an agarose gel. As few as 15 cycles of PCR and lower annealing temperatures were also tested and produced good results, although care should be taken when reducing cycle number to ensure that sufficient product quantity is obtained to enable purification and any desired quality control steps prior to sequencing. Each PCR was performed in triplicate to assess replicability. The PCR products were then purified by SPRI (Agentcourt, 1:1 sample:reagent ratio) and quantified with the Qubit fluorescence assay (Qubit dsDNA HS Assay Kit, ThermoFisher Scientific).

Sequencing and primary data processing. Target-enriched single-cell RNA-seq libraries were loaded at 1.8 pM on a DNA sequencer (Illumina Miniseq) where read 1 (26 bp) sequenced bases in the cell barcode and UMI and read 2 (124 bp) sequenced bases in the transcript. Primary processing of the raw data was conducted using the CellRanger pipeline (10× Genomics). Secondary analyses were carried out using custom Python scripts. The custom scripts used for secondary analysis can be found at (https://github.com/nranu/SC_enrichment). Replicate sequence reads were aggregated by unique molecular identifier (UMI) with secondary analysis operating on UMI counts. Any UMI that received two or fewer reads was removed prior to secondary analysis.

Correlation analysis and Bootstrapping. Gene expression profiles of a given cell were compared before and after enrichment by computing Pearson correlation coefficients. Correlation coefficients were calculated using the expression profiles of targeted single cells in the enriched libraries and the corresponding expression profiles within the original library. One thousand Bootstrap read samples were then generated from each dataset to enable comparing pre-enriched single-cell datasets against themselves. Bootstrap samples of both pre- and post-enrichment data matched the read depth present in the pre-enrichment library for each cell. To determine the highest expected correlation coefficient values given the statistical noise from read and UMI counting, correlations were computed among Bootstrap replicates from the pre-enrichment data derived from the same cells.

Principal components analysis (PCA) and clustering. Feature selection was performed by excluding genes detected in fewer than three cells and removing genes that had low coefficients of variation with a nonparametric Loess regression using a window of 33%. This selection identified ˜1000 highly variable genes that were well-represented in the dataset. Next, the UMI counts per cell were normalized by the median of UMI counts across all cells and log 2 transformed with a pseudocount of 1 and finally, Z-transformed. PCA was performed with the original deeply sequenced library as a training set with the enriched data subsequently projected onto the components defined in analysis of the original library.

Targeting putative AXL+ SIGLEC6+DC (AS DC) cells. To identify AS DC ‘purity scores’, Applicants used a previously described signature scoring system (11). Briefly, Applicants assigned a quantitative score to each cell based on the overall expression of a pre-defined signature gene set after correcting for ‘drop-out’ effects that commonly characterize single cell data (10). The reported AS DC population purity score was based on the top 10 most discriminative genes previously reported: AXL, PPP1R14A, SIGLEC6, CD22, DAB2, S100A10, FAM105A, MED12L, ALDH2 and LTK. This ‘purity score’ was used to identify the most likely AS DC candidate cells in the HLA-DR+ 10× library. Note that not all of the 10 classifier-genes were expressed across the putative AS DC candidates in the 10× library, which could be explained by different dropout rates characterizing the 10× library and Smart-Seq2 libraries, the latter having been used in the original AS DC discovery and characterization study (Science. 2017 Apr. 21; 356(6335). pii: eaah4573. doi: 10.1126/science.aah4573).

Understanding biological systems at a single cell resolution may reveal several novel insights which remain masked by the conventional population-based techniques providing an average readout of the behavior of cells. Single-cell transcriptome sequencing holds the potential to identify novel cell types and characterize the cellular composition of any organ or tissue in health and disease. Sagar et al. (Methods Mol Biol. 2018; 1766:257-283. doi: 10.1007/978-1-4939-7768-0_15) describe a customized high-throughput protocol for single-cell RNA-sequencing (scRNA-seq) combining flow cytometry and a nanoliter-scale robotic system. Since scRNA-seq requires amplification of a low amount of endogenous cellular RNA, leading to substantial technical noise in the dataset, downstream data filtering and analysis require special care. Sagar et al. also briefly describe in-house state-of-the-art data analysis algorithms developed to identify cellular subpopulations including rare cell types as well as to derive lineage trees by ordering the identified subpopulations of cells along the inferred differentiation trajectories.

Han et al. (Genome Biol. 2018; 19: 47) use high throughput single-cell RNA-sequencing (scRNA-seq), based on optimized microfluidic circuits, to profile early differentiation lineages in the human embryoid body system. Han et al. used Fluidigm C1 system and C1 high-throughput integrated fluidics circuits (HT IFCs) to perform the single-cells capture and library construction. A total of 4000-8000 cells were loaded onto a medium-sized (10-17 μm) HT IFCs. The efficiency of capture was measured under the microscope. The capture sites without cell or with more than one cell were marked and excluded from further analysis. C1 system captured and converted all polyadenylated messenger RNA (mRNA) into cDNA with the cell-specific barcodes. After reverse transcription and preamplification, cDNA was prepared as samples for next-generation sequencing using library tagmentation and 3′ end enrichment. Samples harvested from HT IFCs were used to create libraries for Illumina sequencing with an Illumina Nextera XT DNA Library kit.

### High Throughput Droplet Single-Cell Genotyping of Transcriptomes (GoT)

Defining the transcriptomic identity of clonally related malignant cells is challenging in the absence of cell surface markers that distinguish cancer clones from one another or from admixed non-neoplastic cells. While single-cell methods have been devised to capture both the transcriptome and genotype, these methods are not compatible with droplet-based single-cell transcriptomics, limiting their throughput. To overcome this limitation, Nam et al. (https://doi.org/10.1101/444687) present single-cell Genotyping of Transcriptomes (GoT), which integrates cDNA genotyping with high-throughput droplet-based single-cell RNA-seq. Nam et al. further demonstrate that multiplexed GoT can interrogate multiple genotypes for distinguishing subclonal transcriptomic identity. Nam et al. apply GoT to 26,039 CD34+ cells across six patients with myeloid neoplasms, in which the complex process of hematopoiesis is corrupted by CALR-mutated stem and progenitor cells. Nam et al. define high-resolution maps of malignant versus normal hematopoietic progenitors, and show that while mutant cells are comingled with wildtype cells throughout the hematopoietic progenitor landscape, their frequency increases with differentiation. Nam et al. identify the unfolded protein response as a predominant outcome of CALR mutations, with significant cell identity dependency. Furthermore, Nam et al. identify that CALR mutations lead to NF-KB pathway upregulation specifically in uncommitted early stem cells. Collectively, GoT provides high-throughput linkage of single-cell genotypes with transcriptomes and reveals that the transcriptional output of somatic mutations is heavily dependent on the native cell identity.

To link genotypes to single-cell transcriptomes in high throughput droplet-based platforms, Nam et al. devised a strategy to pair targeted genotyping with single-cell whole transcriptomics (GoT). First, Nam et al. add gene-specific primers during the cDNA amplification step of the 10× Chromium procedure to promote amplification of even lowly-expressed genes of interest. Second, after generation of the cDNA library, a small portion of the cDNA (˜10%) is aliquoted for targeted genotyping. Locus specific primers are designed based on known somatic mutations identified from bulk DNA genotyping of the sample, and used to amplify the locus of interest together with the generic forward SI-PCR primer (10× Genomics) to retain the cell barcode (CB) and unique molecule identifier (UMI). The targeted amplicon library is subsequently spiked back into the 10× gene expression library to be sequenced together, or may be alternatively sequenced separately. Finally, Applicants interrogate target amplicon reads for mutation status at the locus of interest, and link the genotype information to single-cell gene expression profiles via shared cell barcode information.

### CRISPR Multiplexing

The aggregation of cellular constituents may be a cell that is a member of a cell population. The cell may be transformed or transduced with one or more genomic sequence-perturbation constructs that perturb a genomic sequence in the cells, wherein each distinct genomic sequence-perturbation construct comprises a unique-perturbation-identifier (UPI) sequence unique to that construct. The genomic sequence-perturbation construct may comprise a sequence encoding a guide RNA sequence of a CRISPR-Cas targeting system. The method may further comprise multiplex transformation of the population of cells with a plurality of genomic sequence-perturbation constructs. The UPI sequence may be attached to a perturbation-sequence-capture sequence, and the microbeads may comprise a perturbation-sequence-capture-binding-sequence having specific binding affinity for the perturbation-sequence-capture sequence attached to the UPI sequence. The UPI sequence may be attached to a universal ligation handle sequence, whereby a USI may be generated by split-pool ligation. The method may further comprise multiplex sequencing of the pooled UCI sequences, USI sequences, and UPI sequences.

The oligonucleotide label may comprise a regulatory sequence configured for amplification by an RNA polymerase, such as T7 polymerase. The labeling ligands may comprise oligonucleotide sequences configured to hybridize to a transcript specific region. The oligonucleotide label may further comprise attachment chemistry, such as an acrylic phosphoramidite modification, whereby the modification allows for incorporation into the polymer matrices upon polymerization. The acrylic phosphoramidite may be Acrydite™ (Eurofins Scientific, Luxembourg). The method may further comprise amplification of the oligonucleotide label and USI by PCR or T7 amplification before sequencing. T7 amplification may be followed by cDNA generation and optionally amplification by PCR. The oligonucleotide label may further comprise at least one spacer sequence, preferably two spacer sequences. The oligonucleotide label may further comprise a photocleavable linker. The oligonucleotide label may further comprise a restriction enzyme site between the labeling ligand and UCI.

The discrete polymer matrices may be labeled and washed more than once. Discrete polymer matrices may be labeled with a marker specific for a cell type or cell cycle marker or developmental marker, or differentiation marker, or disease marker. The label may be a fluorescent label. The fluorescent label may be used to separate the discrete polymer matrices into distinct groups. The label may be used to identify a certain cell type prior to embedding it into a discrete polymer matrix. The discrete polymer matrices of a distinct group may then be labeled again with labeling ligands that contain an oligonucleotide label of the present invention. After novel information is obtained from the multiplex assay of the present invention, a ‘banked’ population of polymer matrices can be stained for newly identified markers and the population of interest can be sorted (enriched) for, and investigated more deeply.

The aggregation of cellular constituents may be a cell that is a member of a cell population. The cell may be transformed or transduced with one or more genomic sequence-perturbation constructs that perturb a genomic sequence in the cells, wherein each distinct genomic sequence-perturbation construct comprises a unique-perturbation-identifier (UPI) sequence unique to that construct. The genomic sequence-perturbation construct may comprise a sequence encoding a guide RNA sequence of a CRISPR-Cas targeting system. The method may further comprise multiplex transformation of the population of cells with a plurality of genomic sequence-perturbation constructs. The UPI sequence may be attached to a perturbation-sequence-capture sequence, and the microbeads may comprise a perturbation-sequence-capture-binding-sequence having specific binding affinity for the perturbation-sequence-capture sequence attached to the UPI sequence. The UPI sequence may be attached to a universal ligation handle sequence, whereby a USI may be generated by split-pool ligation. The method may further comprise multiplex sequencing of the pooled UCI sequences, USI sequences, and UPI sequences.

The oligonucleotide label may comprise a regulatory sequence configured for amplification by an RNA polymerase, such as T7 polymerase. The labeling ligands may comprise oligonucleotide sequences configured to hybridize to a transcript specific region. The oligonucleotide label may further comprise attachment chemistry, such as an acrylic phosphoramidite modification, whereby the modification allows for incorporation into the polymer matrices upon polymerization. The acrylic phosphoramidite may be Acrydite™ (Eurofins Scientific, Luxembourg). The method may further comprise amplification of the oligonucleotide label and USI by PCR or T7 amplification before sequencing. T7 amplification may be followed by cDNA generation and optionally amplification by PCR. The oligonucleotide label may further comprise at least one spacer sequence, preferably two spacer sequences. The oligonucleotide label may further comprise a photocleavable linker. The oligonucleotide label may further comprise a restriction enzyme site between the labeling ligand and UCI.

The discrete polymer matrices may be labeled and washed more than once. Discrete polymer matrices may be labeled with a marker specific for a cell type or cell cycle marker or developmental marker, or differentiation marker, or disease marker. The label may be a fluorescent label. The fluorescent label may be used to separate the discrete polymer matrices into distinct groups. The label may be used to identify a certain cell type prior to embedding it into a discrete polymer matrix. The discrete polymer matrices of a distinct group may then be labeled again with labeling ligands that contain an oligonucleotide label of the present invention. After novel information is obtained from the multiplex assay of the present invention, a ‘banked’ population of polymer matrices can be stained for newly identified markers and the population of interest can be sorted (enriched) for, and investigated more deeply.

The cell(s) may be a member of a cell population, further comprising transforming or transducing the cell population with one or more genomic sequence-perturbation constructs that perturb a genomic sequence in the cells, wherein each distinct genomic sequence-perturbation construct comprises a unique-perturbation-identified (UPI) sequence unique to that construct. The genomic sequence-perturbation construct may comprise a sequence encoding a guide RNA sequence of a CRISPR-Cas targeting system. The method may further comprise multiplex transformation of the population of cells with a plurality of genomic sequence-perturbation constructs. The UPI sequence may be attached to a perturbation-sequence-capture sequence, and the transfer particle may comprise a perturbation-sequence-capture-binding-sequence having specific binding affinity for the perturbation-sequence-capture sequence attached to the UPI sequence. The UPI sequence may be attached to a universal ligation handle sequence, whereby a USI may be generated by split-pool ligation. The method may further comprise multiplex sequencing of the pooled UCI sequences, USI sequences, and UPI sequences.

Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.

### Additional CRISPR-Cas Development and Use Considerations

The present invention may be further illustrated and extended based on aspects of CRISPR-Cas9 development and use as set forth in the following articles and particularly as relates to delivery of a CRISPR protein complex and uses of an RNA guided endonuclease in cells and organisms:
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh 00, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1. [Epub ahead of print].
Gao et al, “Engineered Cpf1 Enzymes with Altered PAM Specificities,” bioRxiv 091611; doi: http://dx.doi.org/10.1101/091611 (Dec. 4, 2016) each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epi static gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and gRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors Applicants developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.

The methods and tools provided herein are exemplified for Cpf1, a type II nuclease that does not make use of tracrRNA. Orthologs of Cpf1 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Casl. In further embodiments, the CRISPR array is used as a seed to identify new effector proteins.

The effectiveness of the present invention has been demonstrated. Preassembled recombinant CRISPR-Cpf1 complexes comprising Cpf1 and crRNA may be transfected, for example by electroporation, resulting in high mutation rates and absence of detectable off-target mutations. Hur, J. K. et al, Targeted mutagenesis in mice by electroporation of Cpf1 ribonucleoproteins, Nat Biotechnol. 2016 Jun. 6. doi: 10.1038/nbt.3596. [Epub ahead of print]. Genome-wide analyses shows that Cpf1 is highly specific. By one measure, in vitro cleavage sites determined for SpCas9 in human HEK293T cells were significantly fewer that for SpCas9. Kim, D. et al., Genome-wide analysis reveals specificities of Cpf1 endonucleases in human cells, Nat Biotechnol. 2016 Jun. 6. doi: 10.1038/nbt.3609. [Epub ahead of print]. An efficient multiplexed system employing Cpf1 has been demonstrated in Drosophila employing gRNAs processed from an array containing inventing tRNAs. Port, F. et al, Expansion of the CRISPR toolbox in an animal with tRNA-flanked Cas9 and Cpf1 gRNAs. doi: http://dx.doi.org/10.1101/046417.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided Fold Nucleases that recognize extended sequences and can edit endogenous genes with high efficiencies in human cells.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014.

Mention is also made of U.S. application 62/180,709, 17-June-15, PROTECTED GUIDE RNAS (PGRNAS); U.S. application 62/091,455, filed, 12-December-14, PROTECTED GUIDE RNAS (PGRNAS); U.S. application 62/096,708, 24-December-14, PROTECTED GUIDE RNAS (PGRNAS); U.S. applications 62/091,462, 12-December-14, 62/096,324, 23-December-14, 62/180,681, 17 Jun. 2015, and 62/237,496, 5 Oct. 2015, DEAD GUIDES FOR CRISPR TRANSCRIPTION FACTORS; U.S. application 62/091,456, 12-December-14 and 62/180,692, 17 Jun. 2015, ESCORTED AND FUNCTIONALIZED GUIDES FOR CRISPR-CAS SYSTEMS; U.S. application 62/091,461, 12-December-14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19-December-14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24-December-14, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-F EB-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 14, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

In addition, mention is made of PCT application PCT/US14/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, and of PCT application PCT/US14/70127, Attorney Reference 47627.99.2091 and BI-2013/101 entitled “DELIVERY, USE AND THERAPEUTIC APPLICATIONS OF THE CRISPR-CAS SYSTEMS AND COMPOSITIONS FOR GENOME EDITING” (claiming priority from one or more or all of US provisional patent applications: 61/915,176; 61/915,192; 61/915,215; 61/915,107, 61/915,145; 61/915,148; and 61/915,153 each filed Dec. 12, 2013) (“the Eye PCT”), incorporated herein by reference, with respect to a method of preparing an sgRNA-and-Cpf1 protein containing particle comprising admixing a mixture comprising an sgRNA and Cpf1 protein (and optionally HDR template).

### Pharmaceutical Compositions/Methods of Delivery

The present invention is also directed to pharmaceutical compositions comprising an effective amount of one or more neoantigenic peptides as described herein (including a pharmaceutically acceptable salt, thereof), optionally in combination with a pharmaceutically acceptable carrier, excipient or additive.

The present invention provides an immunogenic pharmaceutical composition comprising at least one neoantigen obtained from any method described herein or at least one polynucleotide that is expressed in vivo in the subject that encodes the neoantigen.

In some embodiments, the immunogenic pharmaceutical composition comprises a plurality of neoantigens or a plurality of polynucleotides that are expressed in vivo in the subject that encode the neoantigens.

In some embodiments, the immunogenic pharmaceutical composition comprises at least 4 neoantigens or polynucleotides encoding at least 4 neoantigens.

In some embodiments, the immunogenic pharmaceutical composition can comprise up to 12, up to 16 or up to 20 neoantigens or polynucleotides encoding up to 12, up to 16 or up to 20 neoantigens.

In some embodiments, at least one additional neoantigen of the immunogenic composition is ascertained using whole genome sequencing, or mass spectrometry, or any methods described herein.

In some embodiments, the immunogenic composition further comprises an adjuvant. The adjuvant can comprise a TLR-based adjuvant, a mineral oil based adjuvant, or a combination thereof.

In some embodiments, the polynucleotide(s) encoding the neoantigen(s) can be mRNA or DNA.

When administered as a combination, the therapeutic agents (i.e. the neoantigenic peptides) can be formulated as separate compositions that are given at the same time or different times, or the therapeutic agents can be given as a single composition.

In some embodiments, the neoantigen of the immunogenic pharmaceutical composition binds to the HLA protein of the subject with an IC50 of less than 50, 100, 250 or 500 nM and a greater affinity than a corresponding wild-type peptide. The HLA protein of the subject can be a class I HLA protein or a class II HLA protein.

In some embodiments, the neoantigen of the immunogenic pharmaceutical composition has a length of 8 or greater than 8 or 10 or greater than 10 or 15 or greater than 15 or 20 or greater than 20 or 8 to 50 or 15 to 30 or 20 to 40 amino acids.

In some embodiments, the neoantigen of the immunogenic pharmaceutical composition (or a portion thereof) is presented to the subject's immune system by MHC I molecules or by MHC II molecules.

In some embodiments, the neoantigen of the immunogenic pharmaceutical composition elicits an immune response comprising a cytotoxic T cell response, a CD4 or helper T cell response, a CD8 or suppressor T cell response or a combination thereof.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the invention can be used to treat diseases and disease conditions that are acute, and may also be used for treatment of chronic conditions. In particular, the compositions of the invention are used in methods to treat or prevent a neoplasia. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

Surgical resection uses surgery to remove abnormal tissue in cancer, such as mediastinal, neurogenic, or germ cell tumors, or thymoma. In certain embodiments, administration of the composition is initiated following tumor resection. In other embodiments, administration of the neoplasia vaccine or immunogenic composition is initiated 1, 2, 3, 4, 5, 6, 7, 8, 9, 10, 11, 12, 13, 14, 15 or more weeks after tumor resection. Preferably, administration of the neoplasia vaccine or immunogenic composition is initiated 4, 5, 6, 7, 8, 9, 10, 11 or 12 weeks after tumor resection.

Prime/boost regimens refer to the successive administrations of a vaccine or immunogenic or immunological compositions. The term “prime/boost” or “prime/boost dosing regimen” is meant to refer to the successive administrations of a vaccine or immunogenic or immunological compositions. The priming administration (priming) is the administration of a first vaccine or immunogenic or immunological composition type and may comprise one, two or more administrations. The boost administration is the second administration of a vaccine or immunogenic or immunological composition type and may comprise one, two or more administrations, and, for instance, may comprise or consist essentially of annual administrations. In certain embodiments, administration of the neoplasia vaccine or immunogenic composition is in a prime/boost dosing regimen.

In certain embodiments, administration of the neoplasia vaccine or immunogenic composition is in a prime/boost dosing regimen, for example administration of the neoplasia vaccine or immunogenic composition at weeks 1, 2, 3 or 4 as a prime and administration of the neoplasia vaccine or immunogenic composition is at months 2, 3 or 4 as a boost. In another embodiment heterologous prime-boost strategies are used to elicit a greater cytotoxic T-cell response (see Schneider et al., Induction of CD8+ T cells using heterologous prime-boost immunization strategies, Immunological Reviews Volume 170, Issue 1, pages 29-38, August 1999). In another embodiment DNA encoding neoantigens is used to prime followed by a protein boost. In another embodiment protein is used to prime followed by boosting with a virus encoding the neoantigen. In another embodiment a virus encoding the neoantigen is used to prime and another virus is used to boost. In another embodiment protein is used to prime and DNA is used to boost. In a preferred embodiment a DNA vaccine or immunogenic composition is used to prime a T-cell response and a recombinant viral vaccine or immunogenic composition is used to boost the response. In another preferred embodiment a viral vaccine or immunogenic composition is co-administered with a protein or DNA vaccine or immunogenic composition to act as an adjuvant for the protein or DNA vaccine or immunogenic composition. The patient can then be boosted with either the viral vaccine or immunogenic composition, protein, or DNA vaccine or immunogenic composition (see Hutchings et al., Combination of protein and viral vaccines induces potent cellular and humoral immune responses and enhanced protection from murine malaria challenge. Infect Immun. 2007 December; 75(12):5819-26. Epub 2007 Oct. 1).

The pharmaceutical compositions can be processed in accordance with conventional methods of pharmacy to produce medicinal agents for administration to patients in need thereof, including humans and other mammals.

Modifications of the neoantigenic peptides can affect the solubility, bioavailability and rate of metabolism of the peptides, thus providing control over the delivery of the active species. Solubility can be assessed by preparing the neoantigenic peptide and testing according to known methods well within the routine practitioner's skill in the art.

In certain embodiments of the pharmaceutical composition the pharmaceutically acceptable carrier comprises water. In certain embodiments, the pharmaceutically acceptable carrier further comprises dextrose. In certain embodiments, the pharmaceutically acceptable carrier further comprises dimethylsulfoxide. In certain embodiments, the pharmaceutical composition further comprises an immunomodulator or adjuvant. In certain embodiments, the immunodulator or adjuvant is selected from the group consisting of poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In certain embodiments, the immunomodulator or adjuvant comprises poly-ICLC.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine or immunogenic composition of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Dimethylxanthenone-4-Acetic Acid, Journal of Immunology, 190:5216-25 and Kim et al. (2013) Anticancer Flavonoids are Mouse-Selective STING Agonists, 8:1396-1401).

The vaccine or immunological composition may also include an adjuvant compound chosen from the acrylic or methacrylic polymers and the copolymers of maleic anhydride and an alkenyl derivative. It is in particular a polymer of acrylic or methacrylic acid cross-linked with a polyalkenyl ether of a sugar or polyalcohol (carbomer), in particular cross-linked with an allyl sucrose or with allylpentaerythritol. It may also be a copolymer of maleic anhydride and ethylene cross-linked, for example, with divinyl ether (see U.S. Pat. No. 6,713,068 hereby incorporated by reference in its entirety).

In certain embodiments, the pH modifier can stabilize the adjuvant or immunomodulator as described herein.

In certain embodiments, a pharmaceutical composition comprises: one to five peptides, dimethylsulfoxide (DMSO), dextrose, water, succinate, poly I: poly C, poly-L-lysine, carboxymethylcellulose, and chloride. In certain embodiments, each of the one to five peptides is present at a concentration of 300m/ml. In certain embodiments, the pharmaceutical composition comprises ≤3% DMSO by volume. In certain embodiments, the pharmaceutical composition comprises 3.6-3.7% dextrose in water. In certain embodiments, the pharmaceutical composition comprises 3.6-3.7 mM succinate (e.g., as sodium succinate) or a salt thereof. In certain embodiments, the pharmaceutical composition comprises 0.5 mg/ml poly I: poly C. In certain embodiments, the pharmaceutical composition comprises 0.375 mg/ml poly-L-Lysine. In certain embodiments, the pharmaceutical composition comprises 1.25 mg/ml sodium carboxymethylcellulose. In certain embodiments, the pharmaceutical composition comprises 0.225% sodium chloride.

Pharmaceutical compositions comprise the herein-described tumor specific neoantigenic peptides in a therapeutically effective amount for treating diseases and conditions (e.g., a neoplasia/tumor), which have been described herein, optionally in combination with a pharmaceutically acceptable additive, carrier and/or excipient. One of ordinary skill in the art from this disclosure and the knowledge in the art will recognize that a therapeutically effective amount of one of more compounds according to the present invention may vary with the condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

The active compound is included in the pharmaceutically acceptable carrier or diluent in an amount sufficient to deliver to a patient a therapeutically effective amount for the desired indication, without causing serious toxic effects in the patient treated.

Oral compositions generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as a fatty oil. In addition, dosage unit forms can contain various other materials which modify the physical form of the dosage unit, for example, coatings of sugar, shellac, or enteric agents.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose.

In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 1-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier usually comprises sterile water or aqueous sodium chloride solution, though other ingredients including those which aid dispersion may be included. Of course, where sterile water is to be used and maintained as sterile, the compositions and carriers are also sterilized. Injectable suspensions may also be prepared, in which case appropriate liquid carriers, suspending agents and the like may be employed.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub-cutaneous, transdermal (which may include a penetration enhancement agent), buccal and suppository administration, among other routes of administration, including through an eye or ocular route.

The neoplasia vaccine or immunogenic composition, and any additional agents, may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

In certain embodiments, the vaccine or immunogenic composition is administered intravenously or subcutaneously. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The tumor specific neoantigenic peptides may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The tumor specific neoantigenic peptides may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal anti-inflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fluorouracil and methotrexate; DNA antimetabolites such as 5-fluoro-2′-deoxy-uridine, ara-C, hydroxyurea and thioguanine; antibodies such as HERCEPTIN and RITUXAN.

It should be understood that in addition to the ingredients particularly mentioned herein, the formulations of the present invention may include other agents conventional in the art having regard to the type of formulation in question, for example, those suitable for oral administration may include flavoring agents.

Pharmaceutically acceptable salt forms may be the preferred chemical form of compounds according to the present invention for inclusion in pharmaceutical compositions according to the present invention.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. In some embodiments, the method for preparing the neoantigen for the immunogenic pharmaceutical composition further comprises synthesizing the neoantigen. As can be appreciated by the skilled artisan, further methods of synthesizing the compounds of the formulae herein is evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P.G.M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The additional agents that may be included with the tumor specific neo-antigenic peptides of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

### Vaccine or Immunogenic Compositions

The present invention is directed in some aspects to pharmaceutical compositions suitable for the prevention or treatment of cancer. In one embodiment, the composition comprises at least an immunogenic composition, e.g., a neoplasia vaccine or immunogenic composition capable of raising a specific T-cell response. The neoplasia vaccine or immunogenic composition comprises neoantigenic peptides and/or neoantigenic polypeptides corresponding to tumor specific neoantigens as described herein.

A suitable neoplasia vaccine or immunogenic composition can preferably contain a plurality of tumor specific neoantigenic peptides. In an embodiment, the vaccine or immunogenic composition can include between 1 and 100 sets of peptides, more preferably between 1 and 50 such peptides, even more preferably between 10 and 30 sets peptides, even more preferably between 15 and 25 peptides. According to another preferred embodiment, the vaccine or immunogenic composition can include at least one peptides, more preferably 2, 3, 4, or 5 peptides, In certain embodiments, the vaccine or immunogenic composition can comprise 5, 6, 7, 8, 9, 10, 11, 12, 13, 14, 15, 16, 17, 18, 19, 20, 21, 22, 23, 24, 25, 26, 27, 28, 29, or 30 different peptides.

The optimum amount of each peptide to be included in the vaccine or immunogenic composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA injection include i.d., i.m., s.c, i.p. and i.v. For example, doses of between 1 and 500 mg 50 μg and 1.5 mg, preferably 10 μg to 500 of peptide or DNA may be given and can depend from the respective peptide or DNA. Doses of this range were successfully used in previous trials (Brunsvig P F, et al., Cancer Immunol Immunother. 2006; 55(12): 1553-1564; M. Staehler, et al., ASCO meeting 2007; Abstract No 3017). Other methods of administration of the vaccine or immunogenic composition are known to those skilled in the art.

In one embodiment of the present invention the different tumor specific neoantigenic peptides and/or polypeptides are selected for use in the neoplasia vaccine or immunogenic composition so as to maximize the likelihood of generating an immune attack against the neoplasias/tumors in a high proportion of subjects in the population. Without being bound by theory, it is believed that the inclusion of a diversity of tumor specific neoantigenic peptides can generate a broad scale immune attack against a neoplasia/tumor. In one embodiment, the selected tumor specific neoantigenic peptides/polypeptides are encoded by missense mutations. In a second embodiment, the selected tumor specific neoantigenic peptides/polypeptides are encoded by a combination of missense mutations and neoORF mutations. In a third embodiment, the selected tumor specific neoantigenic peptides/polypeptides are encoded by neoORF mutations.

In one embodiment in which the selected tumor specific neoantigenic peptides/polypeptides are encoded by missense mutations, the peptides and/or polypeptides are chosen based on their capability to associate with the MHC molecules of a high proportion of subjects in the population. Peptides/polypeptides derived from neoORF mutations can also be selected on the basis of their capability to associate with the MHC molecules of the patient population.

The vaccine or immunogenic composition is capable of raising a specific cytotoxic T-cells response and/or a specific helper T-cell response.

The vaccine or immunogenic composition can further comprise an adjuvant and/or a carrier. Examples of useful adjuvants and carriers are given herein. The peptides and/or polypeptides in the composition can be associated with a carrier such as, e.g., a protein or an antigen-presenting cell such as e.g. a dendritic cell (DC) capable of presenting the peptide to a T-cell.

Adjuvants are any substance whose admixture into the vaccine or immunogenic composition increases or otherwise modifies the immune response to the mutant peptide. Carriers are scaffold structures, for example a polypeptide or a polysaccharide, to which the neoantigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Suitable adjuvants include, but are not limited to 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL. vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS). Recognition of these “danger signals” activates multiple elements of the innate and adaptive immune system. TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endolysosomes. Different TLRs recognize distinct PAMPS. For example, TLR4 is activated by LPS contained in bacterial cell walls, TLR9 is activated by unmethylated bacterial or viral CpG DNA, and TLR3 is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Among the most promising cancer vaccine or immunogenic composition adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-10, as well as maintenance of high levels of co-stimulatory molecules in DCs1. Furthermore, poly-ICLC was recently directly compared to CpG in non-human primates (rhesus macaques) as adjuvant for a protein vaccine or immunogenic composition consisting of human papillomavirus (HPV) 16 capsomers (Stahl-Hennig C, Eisenblatter M, Jasny E, et al. Synthetic double-stranded RNAs are adjuvants for the induction of T helper 1 and humoral immune responses to human papillomavirus in rhesus macaques. PLoS pathogens. April 2009; 5(4)).

CpG immuno stimulatory oligonucleotides have also been reported to enhance the effects of adjuvants in a vaccine or immunogenic composition setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Th1 cells and strong cytotoxic T-lymphocyte (CTL) generation, even in the absence of CD4 T-cell help. The Th1 bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, Jun. 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyI and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′5′-OAS and the P1/eIF2a kinase, also known as the PKR (4-6), as well as RIG-I helicase and MDA5.

In rodents and non-human primates, poly-ICLC was shown to enhance T cell responses to viral antigens, cross-priming, and the induction of tumor-, virus-, and autoantigen-specific CD8+ T-cells. In a recent study in non-human primates, poly-ICLC was found to be essential for the generation of antibody responses and T-cell immunity to DC targeted or non-targeted HIV Gag p24 protein, emphasizing its effectiveness as a vaccine adjuvant.

In human subjects, transcriptional analysis of serial whole blood samples revealed similar gene expression profiles among the 8 healthy human volunteers receiving one single s.c. administration of poly-ICLC and differential expression of up to 212 genes between these 8 subjects versus 4 subjects receiving placebo. Remarkably, comparison of the poly-ICLC gene expression data to previous data from volunteers immunized with the highly effective yellow fever vaccine YF17D showed that a large number of transcriptional and signal transduction canonical pathways, including those of the innate immune system, were similarly upregulated at peak time points.

More recently, an immunologic analysis was reported on patients with ovarian, fallopian tube, and primary peritoneal cancer in second or third complete clinical remission who were treated on a phase 1 study of subcutaneous vaccination with synthetic overlapping long peptides (OLP) from the cancer testis antigen NY-ESO-1 alone or with Montanide-ISA-51, or with 1.4 mg poly-ICLC and Montanide. The generation of NY-ESO-1-specific CD4+ and CD8+ T-cell and antibody responses were markedly enhanced with the addition of poly-ICLC and Montanide compared to OLP alone or OLP and Montanide.

A vaccine or immunogenic composition according to the present invention may comprise more than one different adjuvant. Furthermore, the invention encompasses a therapeutic composition comprising any adjuvant substance including any of those herein discussed. It is also contemplated that the peptide or polypeptide, and the adjuvant can be administered separately in any appropriate sequence.

A carrier may be present independently of an adjuvant. The carrier may be covalently linked to the antigen. A carrier can also be added to the antigen by inserting DNA encoding the carrier in frame with DNA encoding the antigen. The function of a carrier can for example be to confer stability, to increase the biological activity, or to increase serum half-life. Extension of the half-life can help to reduce the number of applications and to lower doses, thus are beneficial for therapeutic but also economic reasons. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier may be a physiologically acceptable carrier acceptable to humans and safe. However, tetanus toxoid and/or diptheria toxoid are suitable carriers in one embodiment of the invention. Alternatively, the carrier may be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments the vaccine or immunogenic composition according to the present invention additionally contains at least one antigen presenting cell.

The antigen-presenting cell (or stimulator cell) typically has an MHC class I or II molecule on its surface, and in one embodiment is substantially incapable of itself loading the MHC class I or II molecule with the selected antigen. As is described in more detail herein, the MHC class I or II molecule may readily be loaded with the selected antigen in vitro.

CD8+ cell activity may be augmented through the use of CD4+ cells. The identification of CD4 T+ cell epitopes for tumor antigens has attracted interest because many immune based therapies against cancer may be more effective if both CD8+ and CD4+ T lymphocytes are used to target a patient's tumor. CD4+ cells are capable of enhancing CD8 T cell responses. Many studies in animal models have clearly demonstrated better results when both CD4+ and CD8+ T cells participate in anti-tumor responses (see e.g., Nishimura et al. (1999) Distinct role of antigen-specific T helper type 1 (TH1) and Th2 cells in tumor eradication in vivo. J Ex Med 190:617-27). Universal CD4+ T cell epitopes have been identified that are applicable to developing therapies against different types of cancer (see e.g., Kobayashi et al. (2008) Current Opinion in Immunology 20:221-27). For example, an HLA-DR restricted helper peptide from tetanus toxoid was used in melanoma vaccines to activate CD4+ T cells non-specifically (see e.g., Slingluff et al. (2007) Immunologic and Clinical Outcomes of a Randomized Phase II Trial of Two Multipeptide Vaccines for Melanoma in the Adjuvant Setting, Clinical Cancer Research 13(21):6386-95). It is contemplated within the scope of the invention that such CD4+ cells may be applicable at three levels that vary in their tumor specificity: 1) a broad level in which universal CD4+ epitopes (e.g., tetanus toxoid) may be used to augment CD8+ cells; 2) an intermediate level in which native, tumor-associated CD4+ epitopes may be used to augment CD8+ cells; and 3) a patient specific level in which neoantigen CD4+ epitopes may be used to augment CD8+ cells in a patient specific manner. Although current algorithms for predicting CD4 epitopes are limited in accuracy, it is a reasonable expectation that many long peptides containing predicted CD8 neoepitopes will also include CD4 epitopes. CD4 epitopes are longer than CD8 epitopes and typically are 10-12 amino acids in length although some can be longer (Kreiter et al, Mutant MHC Class II epitopes drive therapeutic immune responses to cancer, Nature (2015). Thus the neoantigenic epitopes described herein, either in the form of long peptides (>25 amino acids) or nucleic acids encoding such long peptides, may also boost CD4 responses in a tumor and patient-specific manner (level (3) above).

CD8+ cell immunity may also be generated with neoantigen loaded dendritic cell (DC) vaccine. DCs are potent antigen-presenting cells that initiate T cell immunity and can be used as cancer vaccines when loaded with one or more peptides of interest, for example, by direct peptide injection. For example, patients that were newly diagnosed with metastatic melanoma were shown to be immunized against 3 HLA-A*0201-restricted gp100 melanoma antigen-derived peptides with autologous peptide pulsed CD40L/IFN-g-activated mature DCs via an IL-12p70-producing patient DC vaccine (see e.g., Carreno et al (2013) L-12p70-producing patient DC vaccine elicits Tc1-polarized immunity, Journal of Clinical Investigation, 123(8):3383-94 and Ali et al. (2009) In situ regulation of DC subsets and T cells mediates tumor regression in mice, Cancer Immunotherapy, 1(8):1-10). It is contemplated within the scope of the invention that neoantigen loaded DCs may be prepared using the synthetic TLR 3 agonist Polyinosinic-Polycytidylic Acid-poly-L-lysine Carboxymethylcellulose (Poly-ICLC) to stimulate the DCs. Poly-ICLC is a potent individual maturation stimulus for human DCs as assessed by an upregulation of CD83 and CD86, induction of interleukin-12 (IL-12), tumor necrosis factor (TNF), interferon gamma-induced protein 10 (IP-10), interleukin 1 (IL-1), and type I interferons (IFN), and minimal interleukin 10 (IL-10) production. DCs may be differentiated from frozen peripheral blood mononuclear cells (PBMCs) obtained by leukapheresis, while PBMCs may be isolated by Ficoll gradient centrifugation and frozen in aliquots.

Illustratively, the following 7 day activation protocol may be used. Day 1—PBMCs are thawed and plated onto tissue culture flasks to select for monocytes which adhere to the plastic surface after 1-2 hr incubation at 37° C. in the tissue culture incubator. After incubation, the lymphocytes are washed off and the adherent monocytes are cultured for 5 days in the presence of interleukin-4 (IL-4) and granulocyte macrophage-colony stimulating factor (GM-CSF) to differentiate to immature DCs. On Day 6, immature DCs are pulsed with the keyhole limpet hemocyanin (KLH) protein which serves as a control for the quality of the vaccine and may boost the immunogenicity of the vaccine. The DCs are stimulated to mature, loaded with peptide antigens, and incubated overnight. On Day 7, the cells are washed, and frozen in 1 ml aliquots containing 4-20×10(6) cells using a controlled-rate freezer. Lot release testing for the batches of DCs may be performed to meet minimum specifications before the DCs are injected into patients (see e.g., Sabado et al. (2013) Preparation of tumor antigen-loaded mature dendritic cells for immunotherapy, J. Vis Exp. August 1; (78). doi: 10.3791/50085).

A DC vaccine may be incorporated into a scaffold system to facilitate delivery to a patient. Therapeutic treatment of a patients neoplasia with a DC vaccine may utilize a biomaterial system that releases factors that recruit host dendritic cells into the device, differentiates the resident, immature DCs by locally presenting adjuvants (e.g., danger signals) while releasing antigen, and promotes the release of activated, antigen loaded DCs to the lymph nodes (or desired site of action) where the DCs may interact with T cells to generate a potent cytotoxic T lymphocyte response to the cancer neoantigens. Implantable biomaterials may be used to generate a potent cytotoxic T lymphocyte response against a neoplasia in a patient specific manner. The biomaterial-resident dendritic cells may then be activated by exposing them to danger signals mimicking infection, in concert with release of antigen from the biomaterial. The activated dendritic cells then migrate from the biomaterials to lymph nodes to induce a cytotoxic T effector response. This approach has previously been demonstrated to lead to regression of established melanoma in preclinical studies using a lysate prepared from tumor biopsies (see e.g., Ali et al. (2209) In situ regulation of DC subsets and T cells mediates tumor regression in mice, Cancer Immunotherapy 1(8):1-10; Ali et al. (2009) Infection-mimicking materials to program dendritic cells in situ. Nat Mater 8:151-8), and such a vaccine is currently being tested in a Phase I clinical trial recently initiated at the Dana-Farber Cancer Institute. This approach has also been shown to lead to regression of glioblastoma, as well as the induction of a potent memory response to prevent relapse, using the C6 rat glioma model.24 in the current proposal. The ability of such an implantable, biomatrix vaccine delivery scaffold to amplify and sustain tumor specific dendritic cell activation may lead to more robust anti-tumor immunosensitization than can be achieved by traditional subcutaneous or intra-nodal vaccine administrations.

The present invention may include any method for loading a neoantigenic peptide onto a dendritic cell. One such method applicable to the present invention is a microfluidic intracellular delivery system. Such systems cause temporary membrane disruption by rapid mechanical deformation of human and mouse immune cells, thus allowing the intracellular delivery of biomolecules (Sharei et al., 2015, PLOS ONE).

Preferably, the antigen presenting cells are dendritic cells. Suitably, the dendritic cells are autologous dendritic cells that are pulsed with the neoantigenic peptide. The peptide may be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278. In certain embodiments the dendritic cells are targeted using CD141, DEC205, or XCR1 markers. CD141+XCR1+DC's were identified as a subset that may be better suited to the induction of anti-tumor responses (Bachem et al., J. Exp. Med. 207, 1273-1281 (2010); Crozat et al., J. Exp. Med. 207, 1283-1292 (2010); and Gallois & Bhardwaj, Nature Med. 16, 854-856 (2010)).

Thus, in one embodiment of the present invention the vaccine or immunogenic composition containing at least one antigen presenting cell is pulsed or loaded with one or more peptides of the present invention. Alternatively, peripheral blood mononuclear cells (PBMCs) isolated from a patient may be loaded with peptides ex vivo and injected back into the patient. As an alternative the antigen presenting cell comprises an expression construct encoding a peptide of the present invention. The polynucleotide may be any suitable polynucleotide and it is preferred that it is capable of transducing the dendritic cell, thus resulting in the presentation of a peptide and induction of immunity.

The inventive pharmaceutical composition may be compiled so that the selection, number and/or amount of peptides present in the composition covers a high proportion of subjects in the population. The selection may be dependent on the specific type of cancer, the status of the disease, earlier treatment regimens, and, of course, the HLA-haplotypes present in the patient population.

Pharmaceutical compositions comprising the peptide of the invention may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use can depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present invention may generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The invention provides compositions for parenteral administration which comprise a solution of the peptides and vaccine or immunogenic compositions are dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

A liposome suspension containing a peptide may be administered intravenously, locally, topically, etc. in a dose which varies according to, inter alia, the manner of administration, the peptide being delivered, and the stage of the disease being treated. For targeting to the immune cells, a ligand, such as, e.g., antibodies or fragments thereof specific for cell surface determinants of the desired immune system cells, can be incorporated into the liposome.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant can, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

The peptides and polypeptides of the invention can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963).

The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Generally, a plasmid for a vaccine or immunological composition can comprise DNA encoding an antigen (e.g., one or more neoantigens) operatively linked to regulatory sequences which control expression or expression and secretion of the antigen from a host cell, e.g., a mammalian cell; for instance, from upstream to downstream, DNA for a promoter, such as a mammalian virus promoter (e.g., a CMV promoter such as an hCMV or mCMV promoter, e.g., an early-intermediate promoter, or an SV40 promoter—see documents cited or incorporated herein for useful promoters), DNA for a eukaryotic leader peptide for secretion (e.g., tissue plasminogen activator), DNA for the neoantigen(s), and DNA encoding a terminator (e.g., the 3′ UTR transcriptional terminator from the gene encoding Bovine Growth Hormone or bGH polyA). A composition can contain more than one plasmid or vector, whereby each vector contains and expresses a different neoantigen. Mention is also made of Wasmoen U.S. Pat. No. 5,849,303, and Dale U.S. Pat. No. 5,811,104, whose text may be useful. DNA or DNA plasmid formulations can be formulated with or inside cationic lipids; and, as to cationic lipids, as well as adjuvants, mention is also made of Loosmore U.S. Patent Application 2003/0104008. Also, teachings in Audonnet U.S. Pat. Nos. 6,228,846 and 6,159,477 may be relied upon for DNA plasmid teachings that can be employed in constructing and using DNA plasmids that contain and express in vivo.

The nucleic acids can also be delivered complexed to cationic compounds, such as cationic lipids. Lipid-mediated gene delivery methods are described, for instance, in WO1996/18372; WO 1993/24640; Mannino & Gould-Fogerite, BioTechniques 6(7): 682-691 (1988); U.S. Pat. No. 5,279,833; WO 1991/06309; and Feigner et al., Proc. Natl. Acad. Sci. USA 84: 7413-7414 (1987).

RNA encoding the peptide of interest (e.g., mRNA) can also be used for delivery (see, e.g., Kiken et al, 2011; Su et al, 2011; see also U.S. Pat. No. 8,278,036; Halabi et al. J Clin Oncol (2003) 21:1232-1237; Petsch et al, Nature Biotechnology 2012 Dec. 7; 30(12):1210-6).

Viral vectors as described herein can also be used to deliver the neoantigenic peptides of the invention. Vectors can be administered so as to have in vivo expression and response akin to doses and/or responses elicited by antigen administration.

A preferred means of administering nucleic acids encoding the peptide of the invention uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immuno stimulatory sequences (ISSs or CpGs) play a role in the immunogenicity of DNA′ vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, IL12, GM-CSF), cytokine-inducing molecules (e.g. LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-(3) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted herein, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA is introduced into a mammalian cell line that is suitable as a target for standard CTL chromium release assays. The transfection method used is dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope-specific CTL lines. Cytolysis, detected by 51 Cr release, indicates production of WIC presentation of mini gene-encoded CTL epitopes.

In vivo immunogenicity is a second approach for functional testing of minigene DNA formulations. Transgenic mice expressing appropriate human MHC molecules are immunized with the DNA product. The dose and route of administration are formulation dependent (e.g. IM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Peptides may be used to elicit CTL ex vivo, as well. The resulting CTL, can be used to treat chronic tumors in patients in need thereof that do not respond to other conventional forms of therapy, or does not respond to a peptide vaccine approach of therapy. Ex vivo CTL responses to a particular tumor antigen are induced by incubating in tissue culture the patient's CTL precursor cells (CTLp) together with a source of antigen-presenting cells (APC) and the appropriate peptide. After an appropriate incubation time (typically 1-4 weeks), in which the CTLp are activated and mature and expand into effector CTL, the cells are infused back into the patient, where they destroy their specific target cell (i.e., a tumor cell). In order to optimize the in vitro conditions for the generation of specific cytotoxic T cells, the culture of stimulator cells are maintained in an appropriate serum-free medium.

Prior to incubation of the stimulator cells with the cells to be activated, e.g., precursor CD8+ cells, an amount of antigenic peptide is added to the stimulator cell culture, of sufficient quantity to become loaded onto the human Class I molecules to be expressed on the surface of the stimulator cells. In the present invention, a sufficient amount of peptide is an amount that allows about 200, and preferably 200 or more, human Class I WIC molecules loaded with peptide to be expressed on the surface of each stimulator cell. Preferably, the stimulator cells are incubated with >2 μg/ml peptide. For example, the stimulator cells are incubates with >3, 4, 5, 10, 15, or more μg/ml peptide.

Resting or precursor CD8+ cells are then incubated in culture with the appropriate stimulator cells for a time period sufficient to activate the CD8+ cells. Preferably, the CD8+ cells are activated in an antigen-specific manner. The ratio of resting or precursor CD8+(effector) cells to stimulator cells may vary from individual to individual and may further depend upon variables such as the amenability of an individual's lymphocytes to culturing conditions and the nature and severity of the disease condition or other condition for which the within-described treatment modality is used. Preferably, however, the lymphocyte: stimulator cell ratio is in the range of about 30:1 to 300:1. The effector/stimulator culture may be maintained for as long a time as is necessary to stimulate a therapeutically useable or effective number of CD8+ cells.

The induction of CTL in vitro requires the specific recognition of peptides that are bound to allele specific WIC class I molecules on APC. The number of specific MHC/peptide complexes per APC is crucial for the stimulation of CTL, particularly in primary immune responses. While small amounts of peptide/WIC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatability complex molecules on cells allows the induction of primary cytotoxic T lymphocyte responses.

Since mutant cell lines do not exist for every human WIC allele, it is advantageous to use a technique to remove endogenous WIC-associated peptides from the surface of APC, followed by loading the resulting empty WIC molecules with the immunogenic peptides of interest. The use of non-transformed (non-tumorigenic), noninfected cells, and preferably, autologous cells of patients as APC is desirable for the design of CTL induction protocols directed towards development of ex vivo CTL therapies. This application discloses methods for stripping the endogenous MHC-associated peptides from the surface of APC followed by the loading of desired peptides.

A stable MHC class I molecule is a trimeric complex formed of the following elements: 1) a peptide usually of 8-10 residues, 2) a transmembrane heavy polymorphic protein chain which bears the peptide-binding site in its a1 and a2 domains, and 3) a non-covalently associated non-polymorphic light chain, p2microglobuiin. Removing the bound peptides and/or dissociating the p2microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destablize p2microglobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell's metabolic rate. It is also likely that cells not actively synthesizing MHC molecules (e.g., resting PBMC) would not produce high amounts of empty surface MHC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic state which is critical for antigen presentation. Mild acid solutions of pH 3 such as glycine or citrate-phosphate buffers have been used to identify endogenous peptides and to identify tumor associated T cell epitopes. The treatment is especially effective, in that only the MHC class I molecules are destabilized (and associated peptides released), while other surface antigens remain intact, including MHC class II molecules. Most importantly, treatment of cells with the mild acid solutions do not affect the cell's viability or metabolic state. The mild acid treatment is rapid since the stripping of the endogenous peptides occurs in two minutes at 4° C. and the APC is ready to perform its function after the appropriate peptides are loaded. The technique is utilized herein to make peptide-specific APCs for the generation of primary antigen-specific CTL. The resulting APC are efficient in inducing peptide-specific CD8+ CTL.

Activated CD8+ cells may be effectively separated from the stimulator cells using one of a variety of known methods. For example, monoclonal antibodies specific for the stimulator cells, for the peptides loaded onto the stimulator cells, or for the CD8+ cells (or a segment thereof) may be utilized to bind their appropriate complementary ligand. Antibody-tagged molecules may then be extracted from the stimulator-effector cell admixture via appropriate means, e.g., via well-known immunoprecipitation or immunoassay methods.

Effective, cytotoxic amounts of the activated CD8+ cells can vary between in vitro and in vivo uses, as well as with the amount and type of cells that are the ultimate target of these killer cells. The amount can also vary depending on the condition of the patient and should be determined via consideration of all appropriate factors by the practitioner. Preferably, however, about 1 ×106 to about 1 ×1012, more preferably about 1 ×108 to about 1 ×1011, and even more preferably, about 1 ×109 to about 1 ×1010 activated CD8+ cells are utilized for adult humans, compared to about 5 ×106-5 ×107 cells used in mice.

Preferably, as discussed herein, the activated CD8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells are not achieved, there is no inherent danger known to be associated with the administration of a small number of stimulator cells, whereas administration of mammalian tumor-promoting cells may be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments are discussed in the sections that follow.

### Vaccine or Immunogenic Composition Adjuvant

Effective vaccine or immunogenic compositions advantageously include a strong adjuvant to initiate an immune response. The adjuvant can be delivered together with, prior to or subsequent to the vaccine or immunogenic compositions. As described herein, poly-ICLC, an agonist of TLR3 and the RNA helicase—domains of MIDAS and RIGS, has shown several desirable properties for a vaccine or immunogenic composition adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NY-ESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile. In addition to a powerful and specific immunogen the neoantigen peptides may be combined with an adjuvant (e.g., poly-ICLC) or another anti-neoplastic agent. Without being bound by theory, these neoantigens are expected to bypass central thymic tolerance (thus allowing stronger anti-tumor T cell response), while reducing the potential for autoimmunity (e.g., by avoiding targeting of normal self-antigens). An effective immune response advantageously includes a strong adjuvant to activate the immune system (Speiser and Romero, Molecularly defined vaccines for cancer immunotherapy, and protective T cell immunity Seminars in Immunol 22:144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, TLR AGONISTS: Are They Good Adjuvants? Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double-stranded RNA induces innate immune responses similar to a live viral vaccine in humans J Exp Med 208:2357 (2011)). In a preferred embodiment Hiltonol®, a GMP preparation of poly-ICLC prepared by Oncovir, Inc, is utilized as the adjuvant. In other embodiments, other adjuvants described herein are envisioned. For instance oil-in-water, water-in-oil or multiphasic W/O/W; see, e.g., U.S. Pat. No. 7,608,279 and Aucouturier et al, Vaccine 19 (2001), 2666-2672, and documents cited therein.

### Dosage

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with neoplasia, e.g. cancer or tumors.

A preferred dose of an agent is the maximum that a patient can tolerate and not develop serious or unacceptable side effects. Exemplary dose ranges include 0.01 mg to 250 mg per day, 0.01 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In embodiments, the agent is administered at a concentration of about 10 micrograms to about 100 mg per kilogram of body weight per day, about 0.1 to about 10 mg/kg per day, or about 1.0 mg to about 10 mg/kg of body weight per day.

In embodiments, the pharmaceutical composition comprises an agent in an amount ranging between 1 and 10 mg, such as 1, 2, 3, 4, 5, 6, 7, 8, 9, or 10 mg.

In embodiments, the therapeutically effective dosage produces a serum concentration of an agent of from about 0.1 ng/ml to about 50-100 mg/ml. The pharmaceutical compositions 5 typically should provide a dosage of from about 0.001 mg to about 2000 mg of compound per kilogram of body weight per day. For example, dosages for systemic administration to a human patient can range from 1-10 mg/kg, 20-80 mg/kg, 5-50 mg/kg, 75-150 mg/kg, 100-500 mg/kg, 250-750 mg/kg, 500-1000 mg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 10 1500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

In embodiments, about 50 nM to about 1 μM of an agent is administered to a subject. In related embodiments, about 50-100 nM, 50-250 nM, 100-500 nM, 250-500 nM, 250-750 nM, 500-750 nM, 500 nM to 1 μM, or 750 nM to 1 μM of an agent is administered to a subject.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

Preferred unit dosage formulations are those containing a daily dose or unit, daily sub-dose, as herein discussed, or an appropriate fraction thereof, of the administered ingredient.

The dosage regimen for treating a disorder or a disease with the tumor specific neoantigenic peptides of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject can depend on a number of factors, such as the mode of administration, the nature of the condition being treated, the body weight of the subject being treated and the judgment of the prescribing physician; all such factors being within the ambit of the skilled artisan from this disclosure and the knowledge in the art.

The amount of compound included within therapeutically active formulations according to the present invention is an effective amount for treating the disease or condition. In general, a therapeutically effective amount of the present preferred compound in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. In its most preferred form, compounds according to the present invention are administered in amounts ranging from about 1 mg/kg/day to about 100 mg/kg/day. The dosage of the compound can depend on the condition being treated, the particular compound, and other clinical factors such as weight and condition of the patient and the route of administration of the compound. It is to be understood that the present invention has application for both human and veterinary use.

For oral administration to humans, a dosage of between approximately 0.1 to 100 mg/kg/day, preferably between approximately 1 and 100 mg/kg/day, is generally sufficient.

Where drug delivery is systemic rather than topical, this dosage range generally produces effective blood level concentrations of active compound ranging from less than about 0.04 to about 400 micrograms/cc or more of blood in the patient. The compound is conveniently administered in any suitable unit dosage form, including but not limited to one containing 0.001 to 3000 mg, preferably 0.05 to 500 mg of active ingredient per unit dosage form. An oral dosage of 10-250 mg is usually convenient.

According to certain exemplary embodiments, the vaccine or immunogenic composition is administered at a dose of about 10 μg to 1 mg per neoantigenic peptide. According to certain exemplary embodiments, the vaccine or immunogenic composition is administered at an average weekly dose level of about 10 μg to 2000 μg per neoantigenic peptide.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The invention provides for pharmaceutical compositions containing at least one tumor specific neoantigen described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, TRANSCUTOL, and AZONE.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as SILASTIC, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the herein-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

### Additional Therapies

The tumor specific neoantigen peptides and pharmaceutical compositions described herein can also be administered in a combination therapy with another agent, for example a therapeutic agent. In certain embodiments, the additional agents can be, but are not limited to, chemotherapeutic agents, anti-angiogenesis agents and agents that reduce immune-suppression.

The neoplasia vaccine or immunogenic composition can be administered before, during, or after administration of the additional agent. In embodiments, the neoplasia vaccine or immunogenic composition is administered before the first administration of the additional agent. In other embodiments, the neoplasia vaccine or immunogenic composition is administered after the first administration of the additional therapeutic agent (e.g., 1, 2, 3, 4, 5, 6, 7, 8, 9, 10, 11, 12, 13, 14 days or more). In embodiments, the neoplasia vaccine or immunogenic composition is administered simultaneously with the first administration of the additional therapeutic agent.

The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, an angiogenesis inhibitor, such ashydroxy angiostatin K1-3, DL-α-Difluoromethyl-ornithine, endostatin, fumagillin, genistein, minocycline, staurosporine, and thalidomide; a DNA intercaltor/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin 15, Nocodazole, Paclitaxel, Podophyllotoxin, Rhizoxin, Vinblastine, Vincristine, Vindesine, and Vinorelbine (Navelbine); and an unclassified therapeutic agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The therapeutic agent may be altretamine, amifostine, asparaginase, capecitabine, cladribine, cisapride, cytarabine, dacarbazine (DTIC), dactinomycin, dronabinol, epoetin alpha, filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride. The therapeutic agent may be a monoclonal antibody or small molecule such as rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 131I-tositumomab, ado-trastuzumab emtansine, lapatinib (Tykerb®), pertuzumab (Perjeta™), ado-trastuzumab emtansine (Kadcyla™) regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™) idelalisib (Zydelig®), crizotinib (Xalkori®), erlotinib (Tarceva®), afatinib dimaleate (Gilotrif®), ceritinib (LDK378/Zykadia), Tositumomab and 131I-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), pembrolizumab (Keytruda®), carfilzomib (Kyprolis®), Ramucirumab (Cyramza™), Cabozantinib (Cometriq™), vandetanib (Caprelsa®), Optionally, the therapeutic agent is a neoantigen. The therapeutic agent may be a cytokine such as interferons (INFs), interleukins (ILs), or hematopoietic growth factors. The therapeutic agent may be INF-α, IL-2, Aldesleukin, IL-2, Erythropoietin, Granulocyte-macrophage colony-stimulating factor (GM-CSF) or granulocyte colony-stimulating factor. The therapeutic agent may be a targeted therapy such as toremifene (Fareston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®). Additionally, the therapeutic agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zolinza), Romidepsin (Istodax), or Ruxolitinib (Jakafi). For prostate cancer treatment, a preferred chemotherapeutic agent with which anti-CTLA-4 can be combined is paclitaxel (TAXOL).

In certain embodiments, the one or more additional agents are one or more anti-glucocorticoid-induced tumor necrosis factor family receptor (GITR) agonistic antibodies. GITR is a costimulatory molecule for T lymphocytes, modulates innate and adaptive immune system and has been found to participate in a variety of immune responses and inflammatory processes. GITR was originally described by Nocentini et al. after being cloned from dexamethasone-treated murine T cell hybridomas (Nocentini et al. Proc Natl Acad Sci USA 94:6216-6221.1997). Unlike CD28 and CTLA-4, GITR has a very low basal expression on naive CD4+ and CD8+ T cells (Ronchetti et al. Eur J Immunol 34:613-622. 2004). The observation that GITR stimulation has immunostimulatory effects in vitro and induced autoimmunity in vivo prompted the investigation of the antitumor potency of triggering this pathway. A review of Modulation Of Ctla 4 And Gitr For Cancer Immunotherapy can be found in Cancer Immunology and Immunotherapy (Avogadri et al. Current Topics in Microbiology and Immunology 344. 2011). Other agents that can contribute to relief of immune suppression include checkpoint inhibitors targeted at another member of the CD28/CTLA4 Ig superfamily such as BTLA, LAG3, ICOS, PDL1 or KIR (Page et a, Annual Review of Medicine 65:27 (2014)). In further additional embodiments, the checkpoint inhibitor is targeted at a member of the TNFR superfamily such as CD40, OX40, CD137, GITR, CD27 or TIM-3. In some cases, targeting a checkpoint inhibitor is accomplished with an inhibitory antibody or similar molecule. In other cases, it is accomplished with an agonist for the target; examples of this class include the stimulatory targets OX40 and GITR.

In certain embodiments, the one or more additional agents are synergistic in that they increase immunogenicity after treatment. In one embodiment the additional agent allows for lower toxicity and/or lower discomfort due to lower doses of the additional therapeutic agents or any components of the combination therapy described herein. In another embodiment the additional agent results in longer lifespan due to increased effectiveness of the combination therapy described herein. Chemotherapeutic treatments that enhance the immunological response in a patient have been reviewed (Zitvogel et al., Immunological aspects of cancer chemotherapy. Nat Rev Immunol. 2008 Jan.; 8(1):59-73). Additionally, chemotherapeutic agents can be administered safely with immunotherapy without inhibiting vaccine specific T-cell responses (Perez et al., A new era in anticancer peptide vaccines. Cancer May 2010). In one embodiment the additional agent is administered to increase the efficacy of the therapy described herein. In one embodiment the additional agent is a chemotherapy treatment. In one embodiment low doses of chemotherapy potentiate delayed-type hypersensitivity (DTH) responses. In one embodiment the chemotherapy agent targets regulatory T-cells. In one embodiment cyclophosphamide is the therapeutic agent. In one embodiment cyclophosphamide is administered prior to vaccination. In one embodiment cyclophosphamide is administered as a single dose before vaccination (Walter et al., Multipeptide immune response to cancer vaccine IMA901 after single-dose cyclophosphamide associates with longer patient survival. Nature Medicine; 18:8 2012). In another embodiment, cyclophosphamide is administered according to a metronomic program, where a daily dose is administered for one month (Ghiringhelli et al., Metronomic cyclophosphamide regimen selectively depletes CD4+CD25+ regulatory T cells and restores T and NK effector functions in end stage cancer patients. Cancer Immunol Immunother 2007 56:641-648). In another embodiment taxanes are administered before vaccination to enhance T-cell and NK-cell functions (Zitvogel et al., 2008). In another embodiment a low dose of a chemotherapeutic agent is administered with the therapy described herein. In one embodiment the chemotherapeutic agent is estramustine. In one embodiment the cancer is hormone resistant prostate cancer. A >50% decrease in serum prostate specific antigen (PSA) was seen in 8.7% of advanced hormone refractory prostate cancer patients by personalized vaccination alone, whereas such a decrease was seen in 54% of patients when the personalized vaccination was combined with a low dose of estramustine (Itoh et al., Personalized peptide vaccines: A new therapeutic modality for cancer. Cancer Sci 2006; 97: 970-976). In another embodiment glucocorticoids are administered with or before the therapy described herein (Zitvogel et al., 2008). In another embodiment glucocorticoids are administered after the therapy described herein. In another embodiment Gemcitabine is administered before, simultaneously, or after the therapy described herein to enhance the frequency of tumor specific CTL precursors (Zitvogel et al., 2008). In another embodiment 5-fluorouracil is administered with the therapy described herein as synergistic effects were seen with a peptide based vaccine (Zitvogel et al., 2008). In another embodiment an inhibitor of Braf, such as Vemurafenib, is used as an additional agent. Braf inhibition has been shown to be associated with an increase in melanoma antigen expression and T-cell infiltrate and a decrease in immunosuppressive cytokines in tumors of treated patients (Frederick et al., BRAF inhibition is associated with enhanced melanoma antigen expression and a more favorable tumor microenvironment in patients with metastatic melanoma. Clin Cancer Res. 2013; 19:1225-1231). In another embodiment an inhibitor of tyrosine kinases is used as an additional agent. In one embodiment the tyrosine kinase inhibitor is used before vaccination with the therapy described herein. In one embodiment the tyrosine kinase inhibitor is used simultaneously with the therapy described herein. In another embodiment the tyrosine kinase inhibitor is used to create a more immune permissive environment. In another embodiment the tyrosine kinase inhibitor is sunitinib or imatinib mesylate. It has previously been shown that favorable outcomes could be achieved with sequential administration of continuous daily dosing of sunitinib and recombinant vaccine (Farsaci et al., Consequence of dose scheduling of sunitinib on host immune response elements and vaccine combination therapy. Int J Cancer; 130: 1948-1959). Sunitinib has also been shown to reverse type-1 immune suppression using a daily dose of 50 mg/day (Finke et al., Sunitinib Reverses Type-1 Immune Suppression and Decreases T-Regulatory Cells in Renal Cell Carcinoma Patients. Clin Cancer Res 2008; 14(20)). In another embodiment targeted therapies are administered in combination with the therapy described herein. Doses of targeted therapies has been described previously (Alvarez, Present and future evolution of advanced breast cancer therapy. Breast Cancer Research 2010, 12(Suppl 2):S1). In another embodiment temozolomide is administered with the therapy described herein. In one embodiment temozolomide is administered at 200 mg/day for 5 days every fourth week of a combination therapy with the therapy described herein. Results of a similar strategy have been shown to have low toxicity (Kyte et al., Telomerase Peptide Vaccination Combined with Temozolomide: A Clinical Trial in Stage IV Melanoma Patients. Clin Cancer Res; 17(13) 2011). In another embodiment the therapy is administered with an additional therapeutic agent that results in lymphopenia. In one embodiment the additional agent is temozolomide. An immune response can still be induced under these conditions (Sampson et al., Greater chemotherapy-induced lymphopenia enhances tumor-specific immune responses that eliminate EGFRvIII-expressing tumor cells in patients with glioblastoma. Neuro-Oncology 13(3):324-333, 2011).

Patients in need thereof may receive a series of priming vaccinations with a mixture of tumor-specific peptides. Additionally, over a 4 week period the priming may be followed by two boosts during a maintenance phase. All vaccinations are subcutaneously delivered. The vaccine or immunogenic composition is evaluated for safety, tolerability, immune response and clinical effect in patients and for feasibility of producing vaccine or immunogenic composition and successfully initiating vaccination within an appropriate time frame. The first cohort can consist of 5 patients, and after safety is adequately demonstrated, an additional cohort of 10 patients may be enrolled. Peripheral blood is extensively monitored for peptide-specific T-cell responses and patients are followed for up to two years to assess disease recurrence.

### Vaccine or Immunogenic Composition Kits and Co-Packaging

In an aspect, the invention provides kits containing any one or more of the elements discussed herein to allow administration of the therapy. The invention provides a kit comprising the neoantigen from any methods described herein or any immunogenic compositions described herein; and an anti-immunosuppressive agent or an anti-immunostimulatory agent or another antineoplastic agent or another cancer therapy.

Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more delivery or storage buffers. Reagents may be provided in a form that is usable in a particular process, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more of the vectors, proteins and/or one or more of the polynucleotides described herein. The kit may advantageously allow the provision of all elements of the systems of the invention. Kits can involve vector(s) and/or particle(s) and/or nanoparticle(s) containing or encoding RNA(s) for 1-50 or more neoantigen mutations to be administered to an animal, mammal, primate, rodent, etc., with such a kit including instructions for administering to such a eukaryote; and such a kit can optionally include any of the anti-cancer agents described herein. The kit may include any of the components above (e.g. vector(s) and/or particle(s) and/or nanoparticle(s) containing or encoding RNA(s) for 1-50 or more neoantigen mutations, neoantigen proteins or peptides) as well as instructions for use with any of the methods of the present invention.

In one embodiment the kit contains at least one vial with an immunogenic composition or vaccine. In one embodiment the kit contains at least one vial with an immunogenic composition or vaccine and at least one vial with an anticancer agent. In one embodiment kits may comprise ready to use components that are mixed and ready to administer. In one aspect a kit contains a ready to use immunogenic or vaccine composition and a ready to use anti-cancer agent. The ready to use immunogenic or vaccine composition may comprise separate vials containing different pools of immunogenic compositions. The immunogenic compositions may comprise one vial containing a viral vector or DNA plasmid and the other vial may comprise immunogenic protein. The ready to use anticancer agent may comprise a cocktail of anticancer agents or a single anticancer agent. Separate vials may contain different anti-cancer agents. In another embodiment a kit may contain a ready to use anti-cancer agent and an immunogenic composition or vaccine in a ready to be reconstituted form. The immunogenic or vaccine composition may be freeze dried or lyophilized. The kit may comprise a separate vial with a reconstitution buffer that can be added to the lyophilized composition so that it is ready to administer. The buffer may advantageously comprise an adjuvant or emulsion according to the present invention. In another embodiment the kit may comprise a ready to reconstitute anti-cancer agent and a ready to reconstitute immunogenic composition or vaccine. In this aspect both may be lyophilized. In this aspect separate reconstitution buffers for each may be included in the kit. The buffer may advantageously comprise an adjuvant or emulsion according to the present invention. In another embodiment the kit may comprise single vials containing a dose of immunogenic composition and anti-cancer agent that are administered together. In another aspect multiple vials are included so that one vial is administered according to a treatment timeline. One vial may only contain the anti-cancer agent for one dose of treatment, another may contain both the anti-cancer agent and immunogenic composition for another dose of treatment, and one vial may only contain the immunogenic composition for yet another dose. In a further aspect the vials are labeled for their proper administration to a patient in need thereof. The immunogen or anti-cancer agents of any embodiment may be in a lyophilized form, a dried form or in aqueous solution as described herein. The immunogen may be a live attenuated virus, protein, or nucleic acid as described herein.

In one embodiment the anticancer agent is one that enhances the immune system to enhance the effectiveness of the immunogenic composition or vaccine. In a preferred embodiment the anti-cancer agent is a checkpoint inhibitor. In another embodiment the kit contains multiple vials of immunogenic compositions and anti-cancer agents to be administered at different time intervals along a treatment plan. In another embodiment the kit may comprise separate vials for an immunogenic composition for use in priming an immune response and another immunogenic composition to be used for boosting. In one aspect the priming immunogenic composition could be DNA or a viral vector and the boosting immunogenic composition may be protein. Either composition may be lyophilized or ready for administering. In another embodiment different cocktails of anti-cancer agents containing at least one anti-cancer agent are included in different vials for administration in a treatment plan.

### EXAMPLES

### Example 1—Translated Unannotated Open Reading Frames Expand the Neoantigen Search Space in Cancer

Genomic aberrations in cancer cells give rise to mutant peptides (neoantigens) displayed on the human leukocyte antigen (HLA) molecules and recognized by T cells, thus triggering an immune response against cancer cells. Patients vaccinated with neoantigen-based peptides display expanded neoantigen-specific T cells, suggesting that this could be a promising avenue for cancer treatment (Ott et al., 2017; Sahin et al., 2017). Neoantigens are commonly predicted based on mutations detected by whole exome sequencing (WES). Their expression levels are estimated using mRNA sequencing (RNA-seq). Ribosome profiling (Ribo-seq) allows to monitor mRNA translation, and has been used to predict a plethora of translated novel unannotated ORFs (nuORFs) (Fields et al., 2015; Ji et al., 2015). Ribo-seq analysis of human fibroblasts infected with HSV-1 and HCMV has identified nuORFs that contribute peptides presented on major histocompatibility complex class I (MHC I) (Erhard et al., 2018), which prompted Applicants to explore nuORFs as a source of neoantigens in cancer. In addition, Ribo-seq was explored as a supplemental resource to RNA-seq, to guage the transcription and translation levels of neoantigens.

Here, Applicants use Ribo-seq, RNA-seq, WES and whole genome sequencing (WGS), whole proteome and MHC I mass spectrometry (MS) to identify thousands of nuORFs that are translated and contribute peptides for MHC I presentation. Applicants further demonstrate that nuORFs can be a significant source of neoantigens in cancer. Finally, Applicants show that RNA-seq in combination with Ribo-seq is a better predictor of neoantigen presentation and immunogenicity than RNA-seq alone.

To determine if nuORFs are a significant source of HLA-presented antigens, Applicants predicted all possible ORFs within transcripts annotated in the GENCODE and MiTranscriptome references. MiTranscriptome is an annotation based on the de novo transcriptome assembly of thousands of RNA-seq libraries from tumors, normal tissues and cell lines (Iyer et al., 2015). Assuming that an ORF must generate a protein that is at least 8 amino acids long for HLA presentation, and that an ORF must start with NTG start codon, there were over 17 million possible ORFs in the annotated transcripts. Given the size of the pan-transcriptome search space, to determine the effect of the database size on the MHC I MS spectra matching and peptide recovery, Applicants constructed additional databases of possible ORFs using Ribo-seq and RNA-seq data from B721.221 cells, which were previously used in mono-allelic MHC I MS studies (Abelin et al., 2017). The RNA-seq and Ribo-seq databases contain all possible ORFs within the reference transcriptomes that are supported by at least 2 RNA-seq or Ribo-seq reads respectively. Applicants also performed Ribo-seq on B721.221 cells and constructed the B721.221 database, which contains all ORFs predicted by RibORF and PRICE tools (Erhard et al., 2018; Ji et al., 2015).

Additionally, Applicants performed Ribo-seq on 28 primary healthy and cancer samples and cell lines, including primary B cells and CLL cells, patient-derived primary glioblastoma and melanoma cell lines and healthy melanocytes, as well as colon carcinoma and melanoma cell lines. Applicants developed a hierarchical ORF prediction pipeline, which allowed us to combine the reads across samples for the maximum ORF calling potential, while also preserving tissue-specific ORFs. Using this pipeline, Applicants generated the PanSample database of predicted ORFs using a combination of RibORF and PRICE tools based on the transcripts annotated in GENCODE and MiTranscriptome references.

The databases contain ORFs of various categories, including previously annotated ORFs, nuORFs derived from the 5′ and 3′ untranslated regions (UTRs) or overlapping, but out-of-frame from annotated ORFs in annotated protein-coding transcripts, long non-coding RNAs (lncRNAs), pseudogenes and other non-coding transcripts (FIG. 1A).

To determine if peptides from nuORFs can be a source of antigens and to estimate the effects of the database size on MS spectra recovery, Applicants searched the collection of mono-allelic MHC class I immunopeptidome mass spectrometry (MS) spectra from 95 common HLA alleles against the five databases Applicants have constructed (Abelin et al., 2017 and unpublished). The PanSample database produced the highest number of identified peptides, while the Transcriptome and RNA-seq databases had the least sensitivity. By sequencing the B721.221 Ribo-seq libraries to saturation and comparing with the peptides found from the PanSample database, Applicants demonstrate that the PanSample database is able to capture all the nuORFs translated and presented on MHC I in B721.221 cells. See Table submitted in ASCII format with the provisional application for all of the ORFs predicted in the PanSample database. Inclusion of overlapping ORFs provides additional rich data for identification of nuORFs compared to other methodologies. Compare, Martinez et al. 2019 Nature Chemical Biology.

Applicants identified 9,454 peptides from 4798 nuORFs of various categories, demonstrating that they are clearly translated. Peptides from nuORFs represent 4.6% of the MHC I immunopeptidome, and 23% of all proteins identified (FIG. 1B).

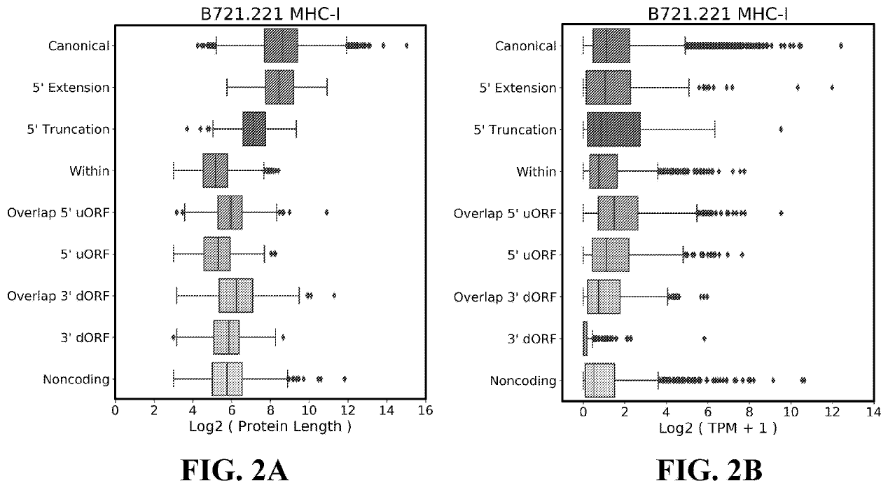

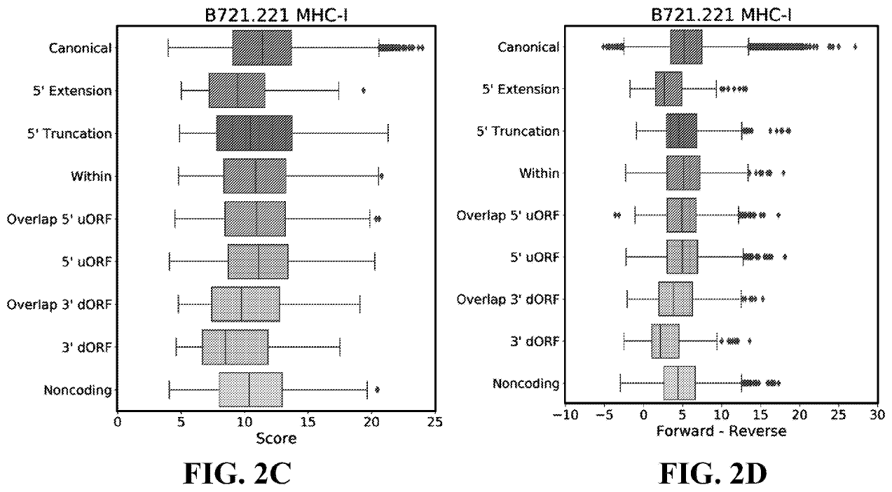

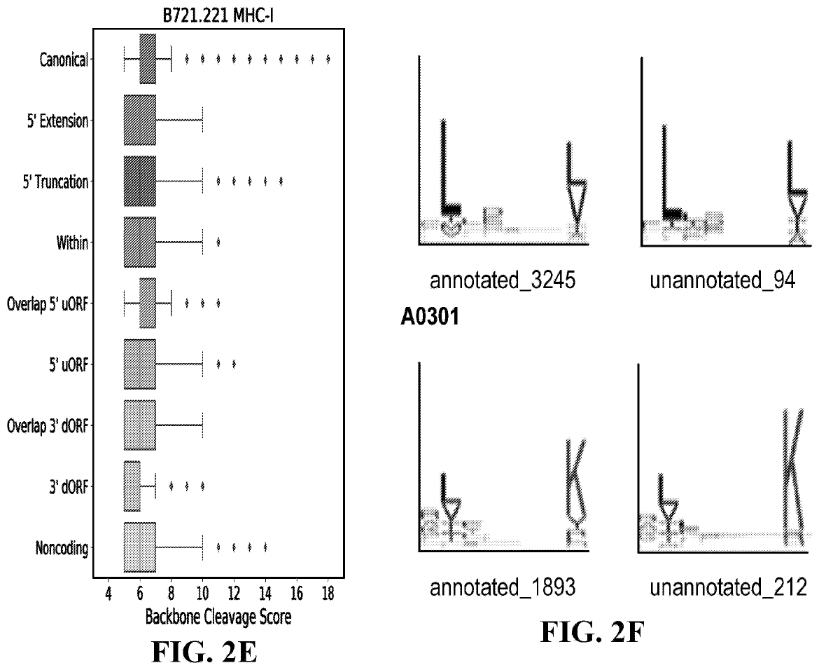

NuORF-derived proteins that contribute peptides to MHC I presentation are significantly shorter than annotated proteins and are overall translated at lower levels (FIG. 2A, B). Peptides from nuORFs and annotated ORFs match in terms of MS score, delta forward reverse score and the backbone cleavage score (FIG. 2C, D), further supporting that nuORFs contribute high quality peptides for HLA presentation. Finally, peptides from nuORFs matched the expected motifs for the alleles where they were detected (FIG. 2E, F).

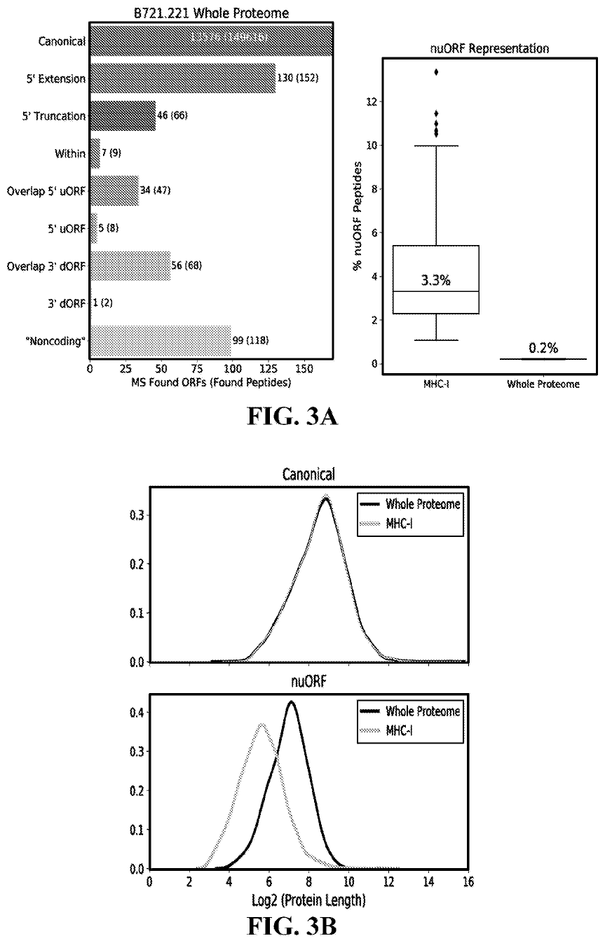

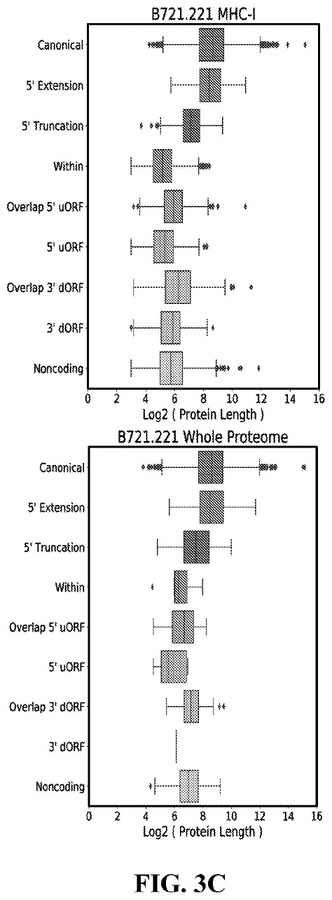

Even though there are thousands of peptides derived from nuORFs identified by MHC I MS, previous whole proteome MS analyses were unable to identify them (Budamgunta et al., 2018). Applicants performed whole proteome MS analyses on B721.221 cells and identified 252 peptides derived from * nuORFs (FIG. 3A,B), which agrees with previous studies, where Ribo-seq predicted nuORFs are under-represented in whole proteome analyses. In particular, short nuORFs, such as uORFs within 5′ UTRs and internal ORFs overlapping, but out-of-frame with the annotated ORFs, were significantly under-represented in the bulk proteomes, compared to longer nuORFs, such as lncRNAs, pseudogenes and other longer nuORFs (FIG. 3C). This observation suggests potentially distinct mechanisms for processing peptides for MHC I presentation derived from short versus long ORFs.

Applicants analyzed MHC I MS data from several cancer patient samples. (FIG. 3D).

To determine if nuORFs are differentially translated across cancer and healthy tissues, Applicants identified nuORFs that are highly upregulated or translated in cancer- and tissue-specific manner that could act as sources of neoantigens even in the absence of somatic mutations. To perform this analysis, Applicants compared the absolute translation levels of nuORFs in cancer samples to healthy tissue samples (such as CLL vs. B cells, and melanoma vs. melanocytes), and to other unrelated cancer and healthy samples. Depending on the cancer type and the quality of the Ribo-seq data, Applicants identified tens to hundreds of nuORFs that are differentially translated in cancer- and patient-specific manner (FIG. 3E). Thus, despite the low number of samples used for this analysis, these preliminary findings demonstrate that Ribo-seq can be a powerful tool to identify nuORFs translated in cancer-specific manner.

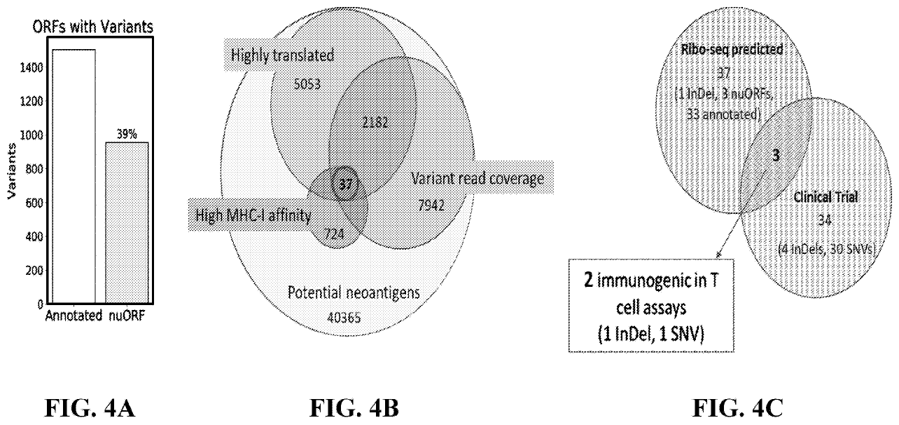

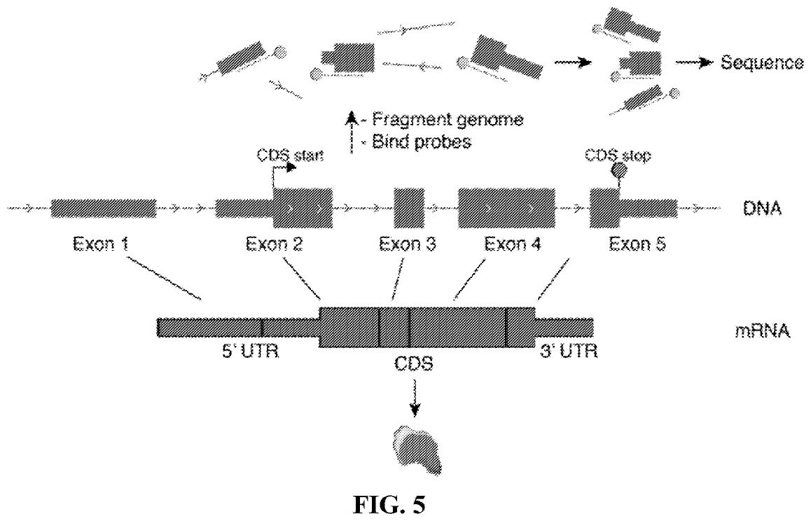

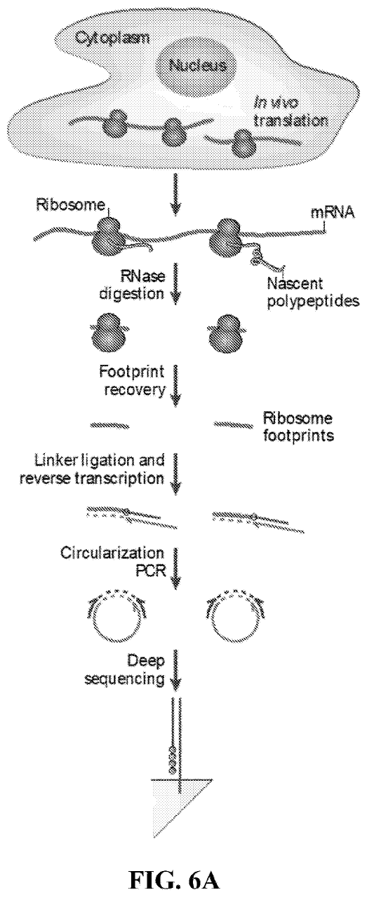

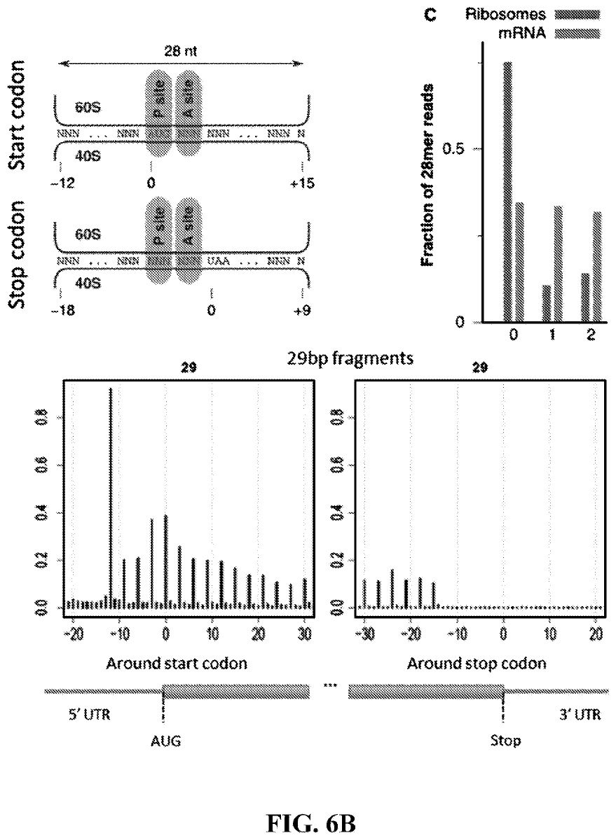

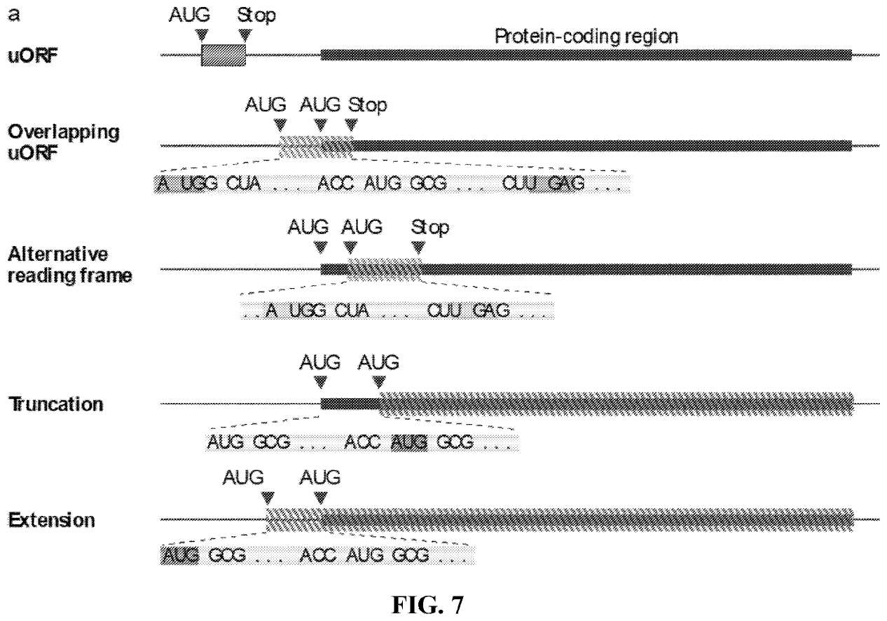

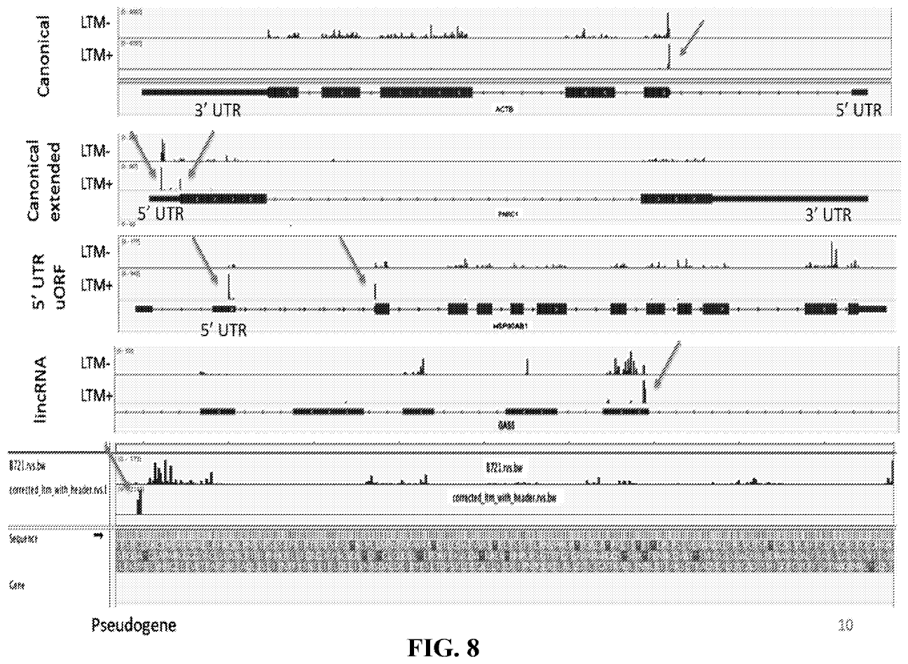

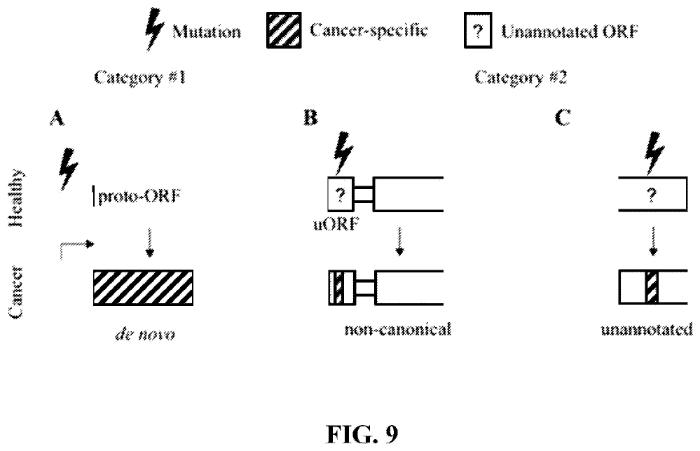

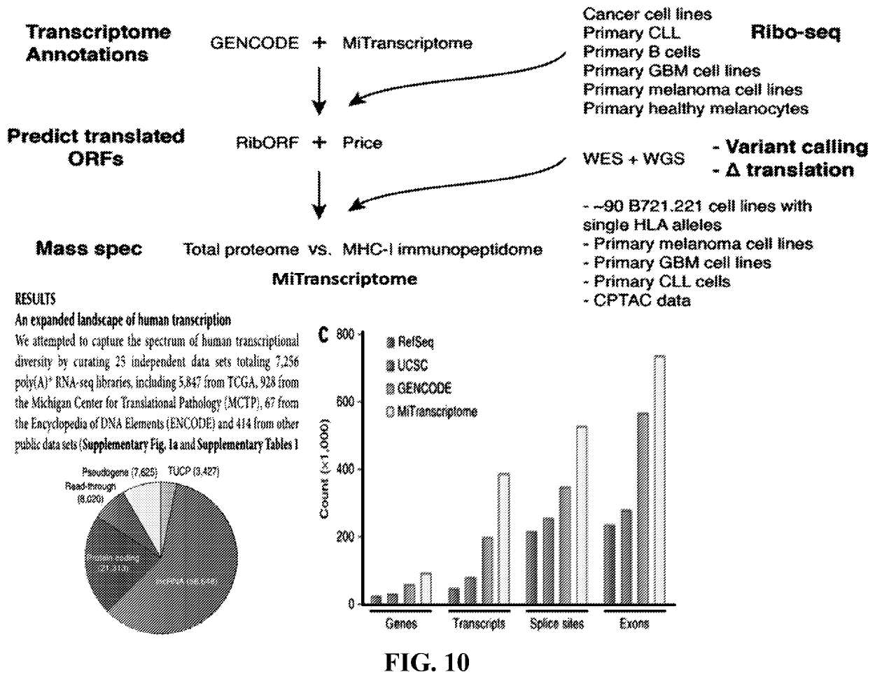

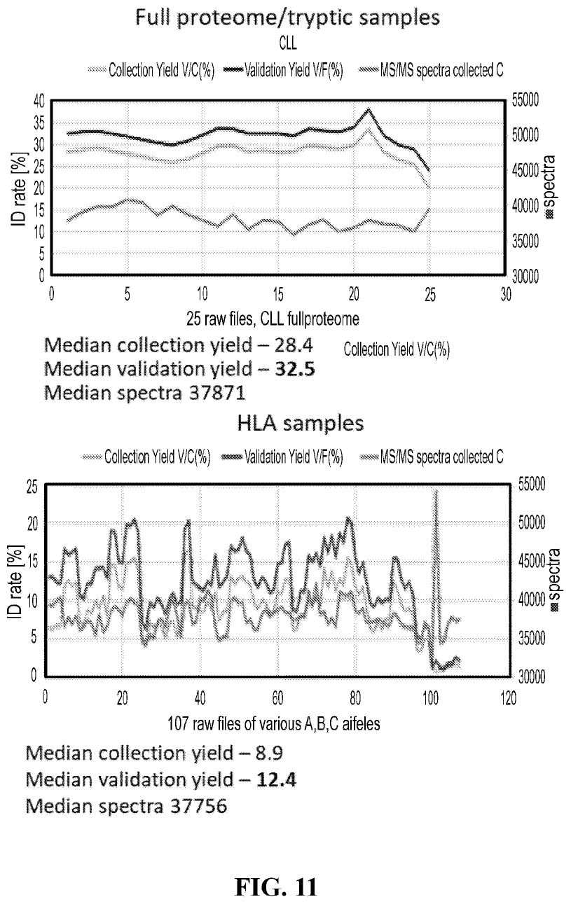

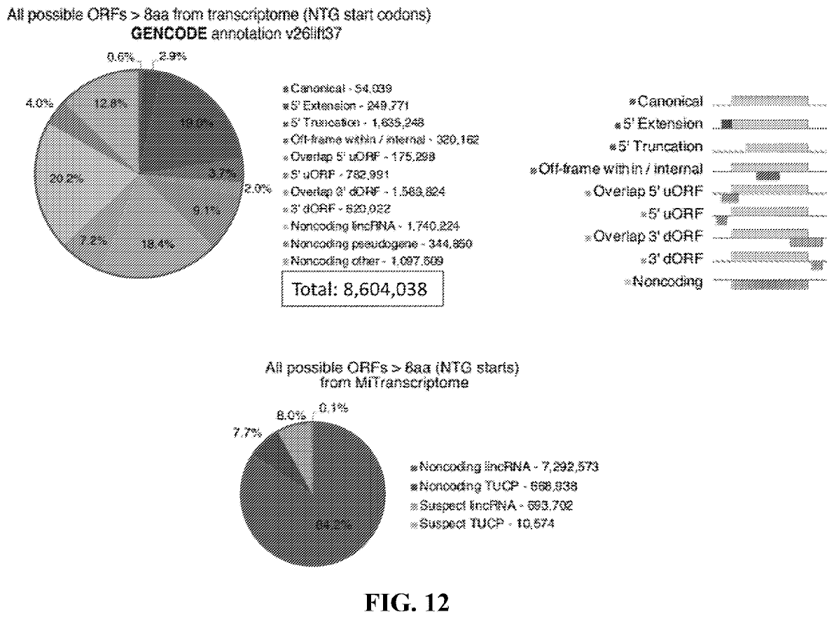

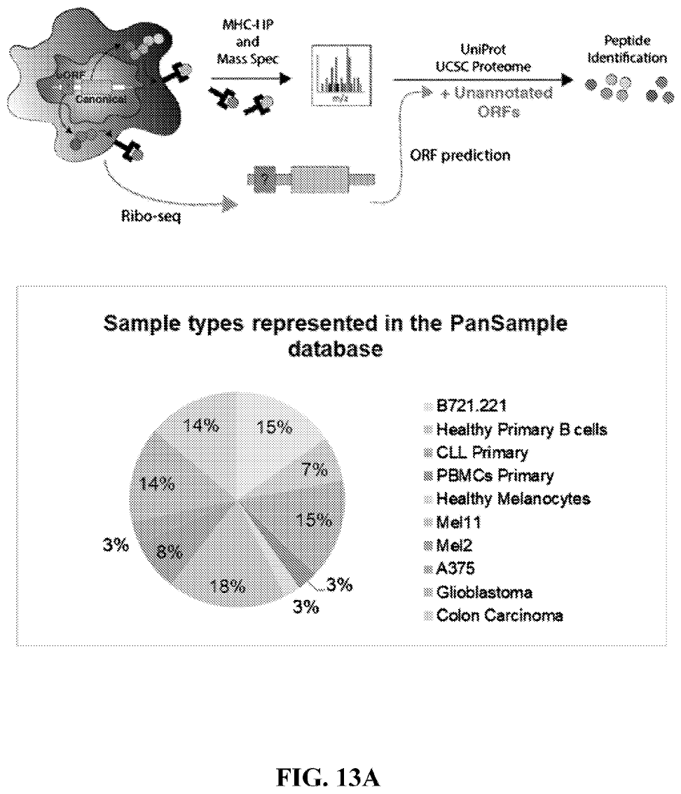

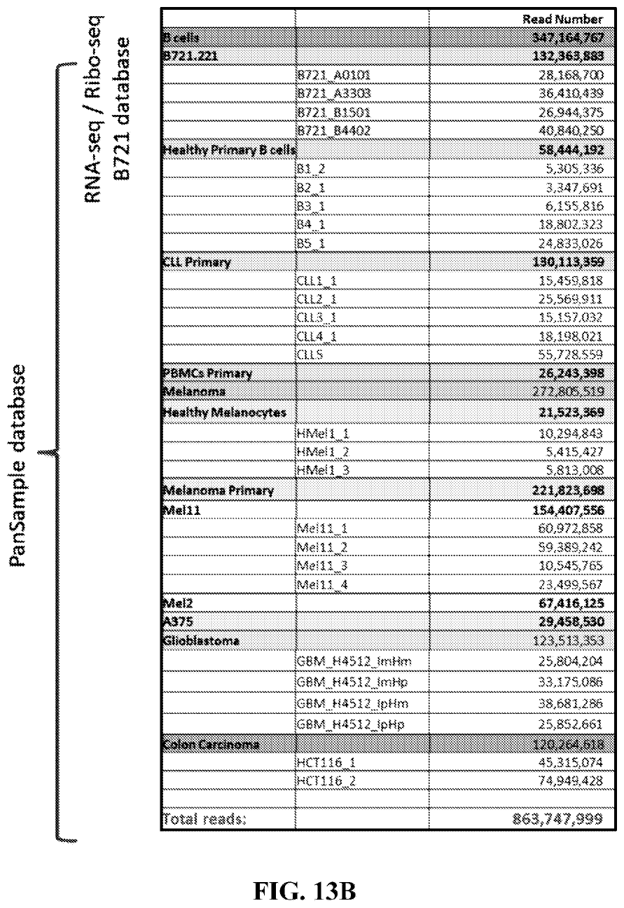

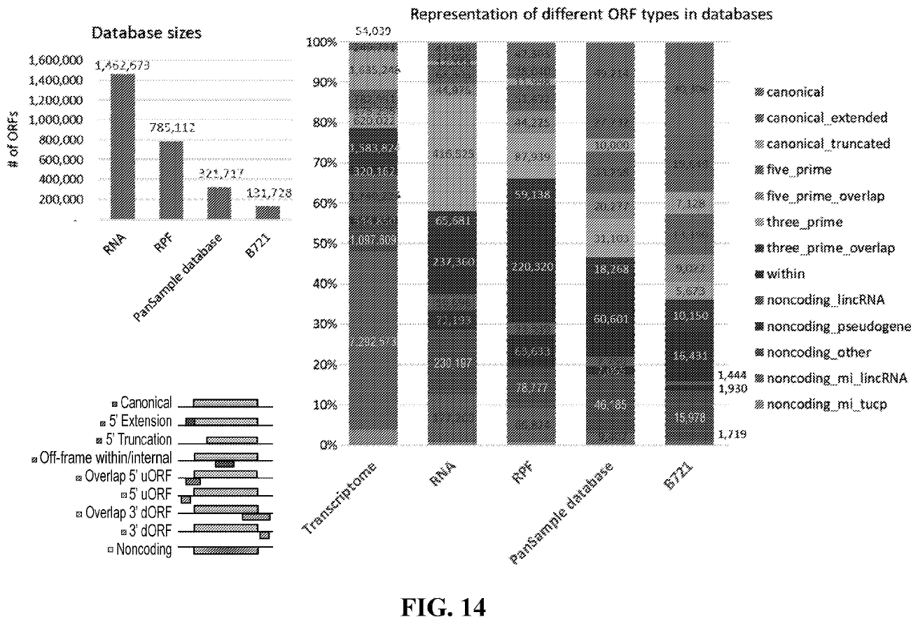

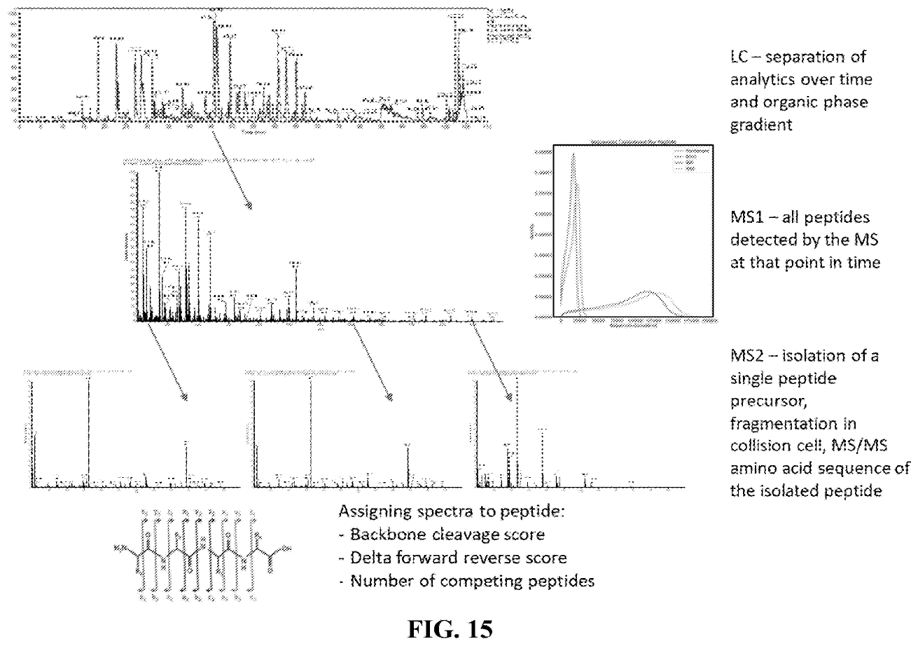

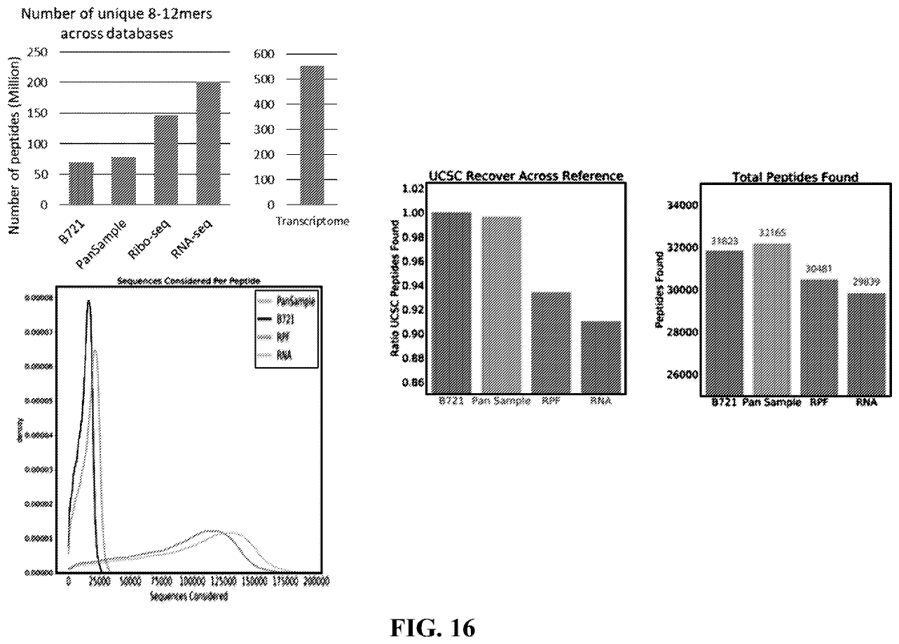

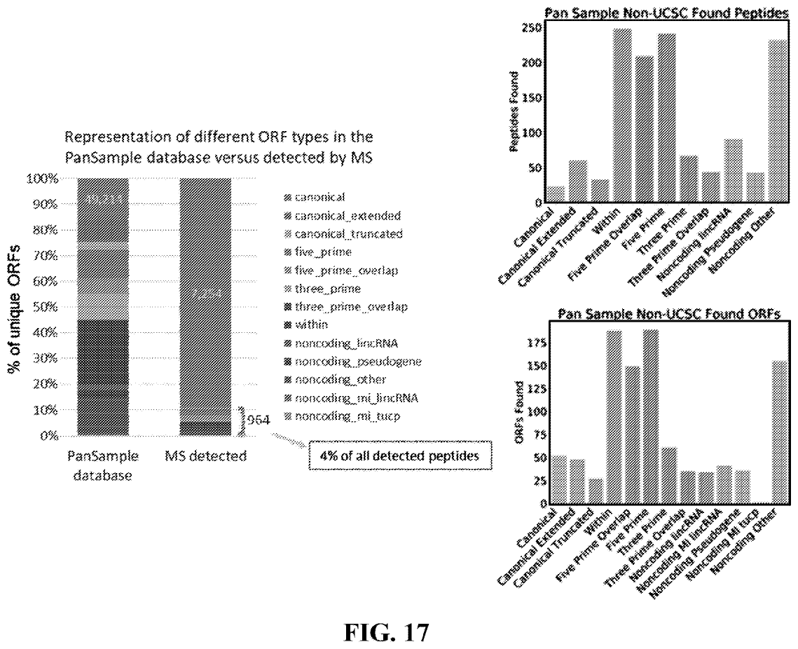

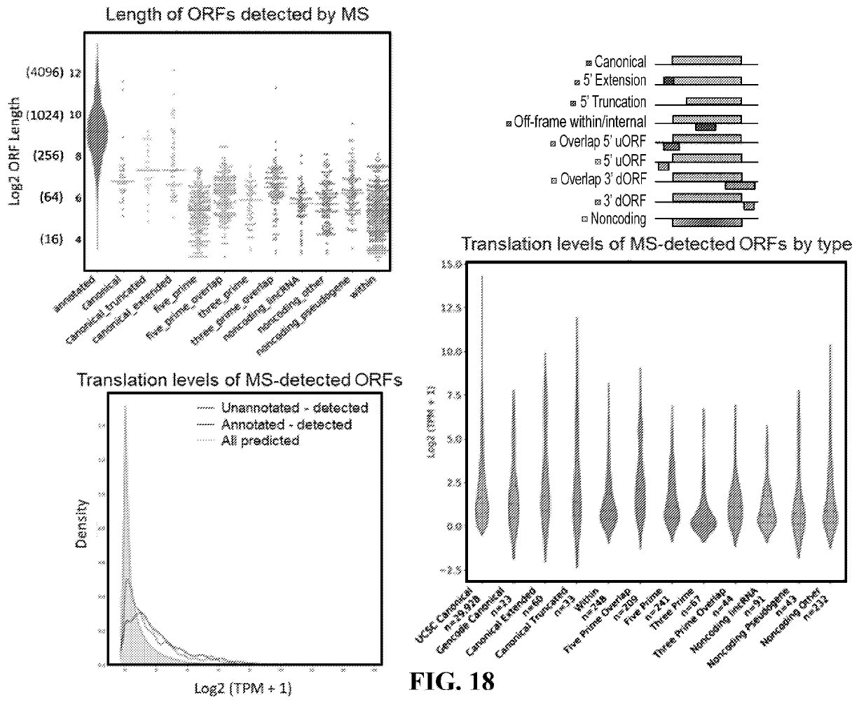

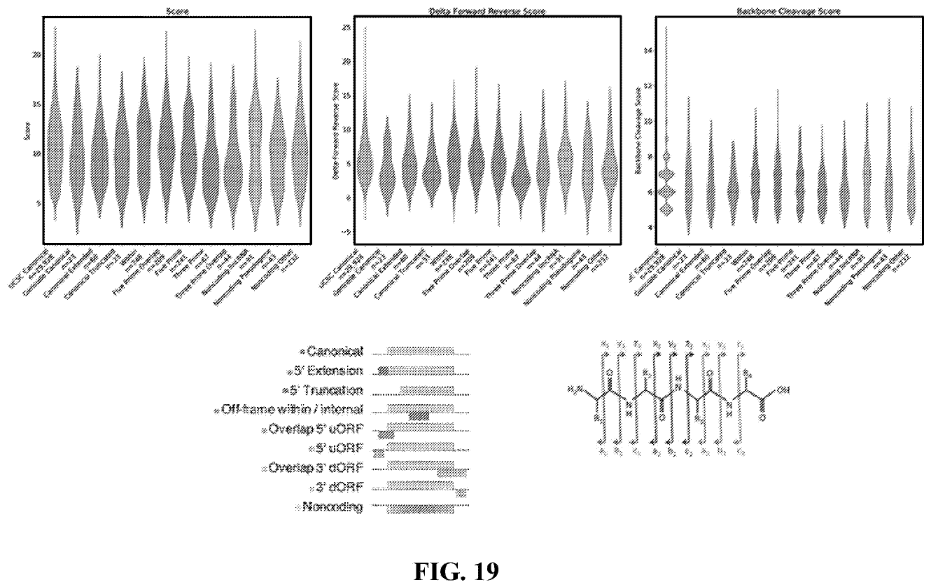

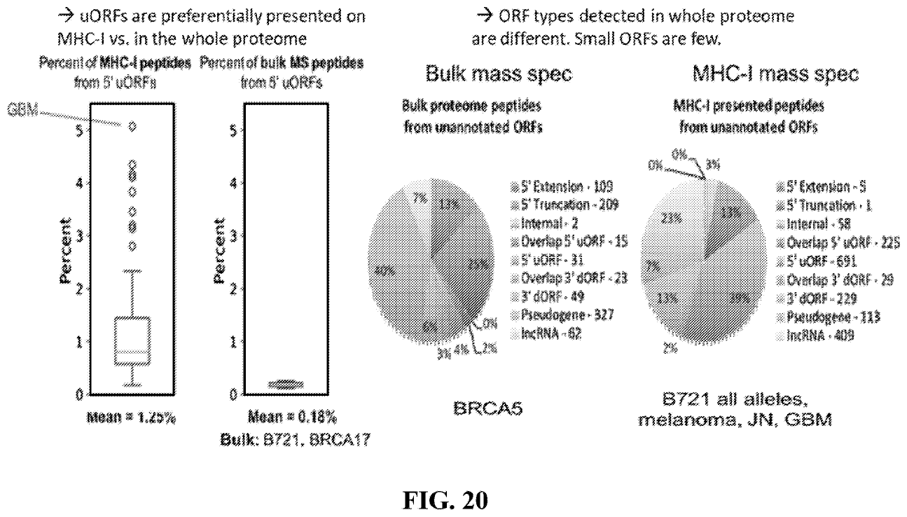

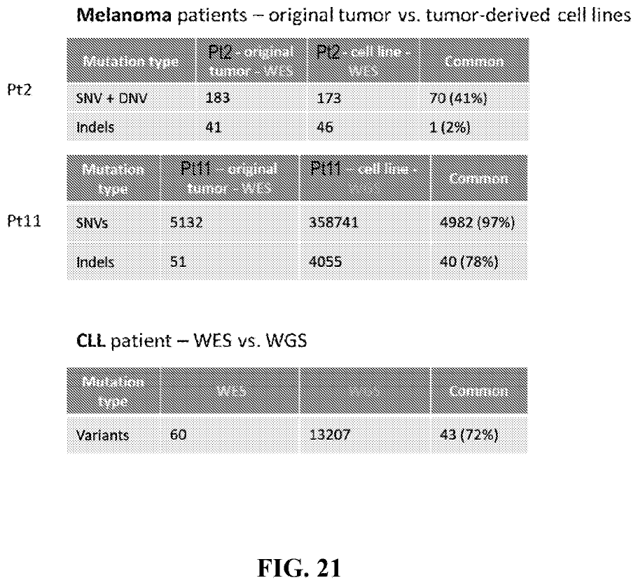

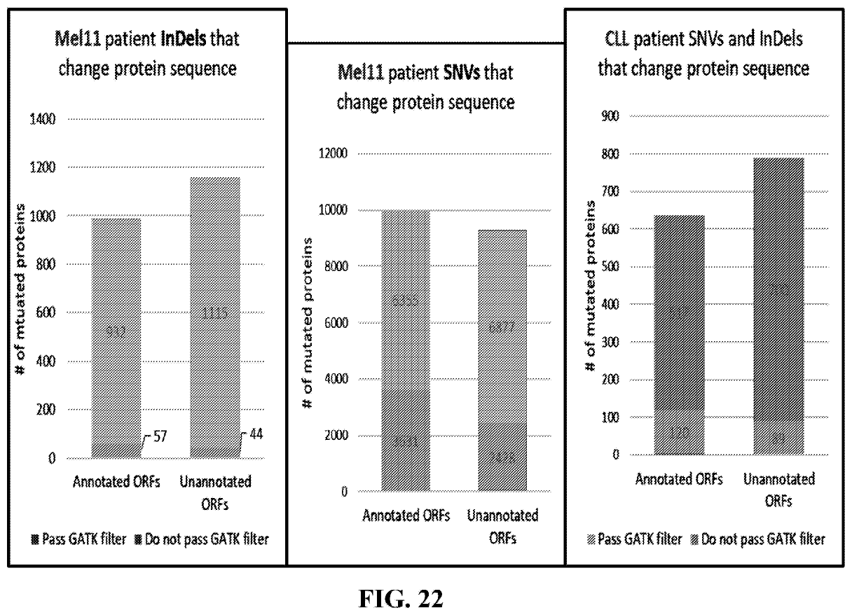

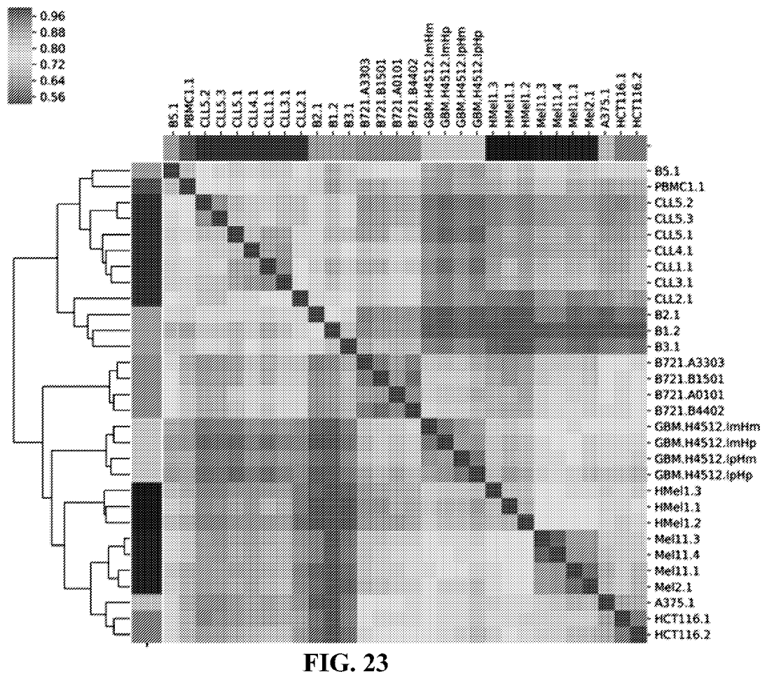

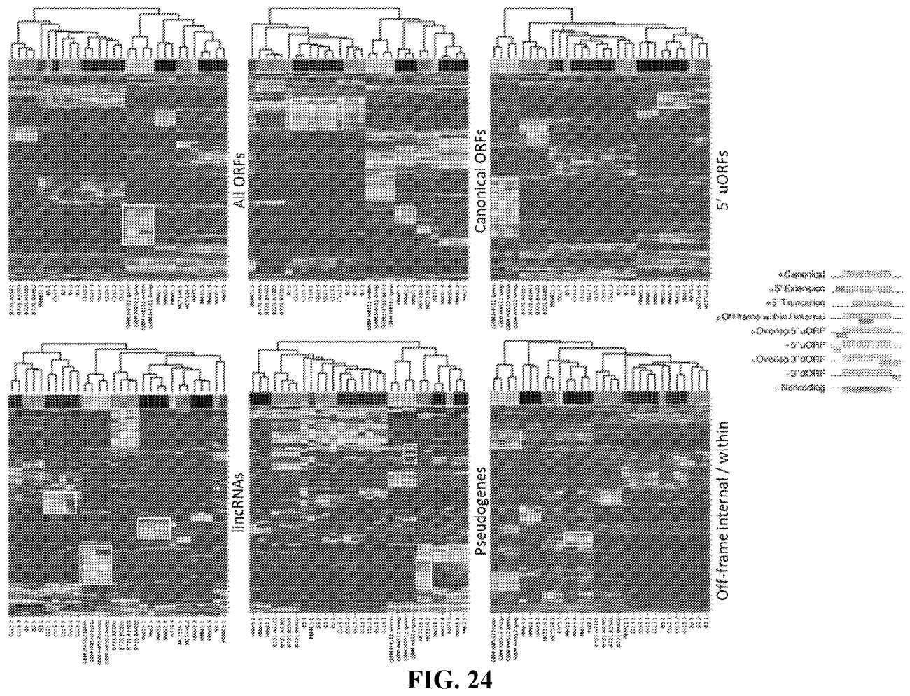

Applicants then proceeded to determine if nuORFs can be a source of neoantigens in cancer. Applicants first evaluated whether WES is sufficient to capture somatic mutations in nuORFs. Applicants then performed WGS on a melanoma patient-derived cell line and matched PBMCs, and a CLL patient blood sample and matched healthy fibroblasts to identify cancer-specific somatic variants. Applicants confirmed that the melanoma patient-derived cell line closely recapitulates the original patient tumor. Applicants mapped somatic mutations to the PanSample ORF database, which contains both annotated ORFs and nuORFs. 39% of somatic mutations mapped to nuORFs (FIG. 4A). Finally, Applicants came up with a set of criteria to prioritize neoantigens for future use, by considering their translation levels, variant coverage and MHC I binding affinity (Abelin et al., 2017; Hoof et al., 2009) (FIG. 4B). Incorporating Ribo-seq data allowed us to select cancer-specific somatic variants that are actually incorporated into translated proteins, increasing the likelihood of them being presented on MHC I. To determine whether cancer-specific variants are incorporated into translated ORFs, Applicants designed a computational pipeline to obtain the number of reads supporting the transcription and translation of the mutant and wild-type alleles, both for single nucleotide variants (SNVs) and insertions and deletions (indels). Applicants re-analyzed the variants selected for this melanoma patient in the neoantigen vaccine clinical trial and demonstrated that the variants that successfully triggered an immune response T cell assays in vitro had higher Ribo-seq coverage and absolute translation than the variants that could not be validated (FIG. 4C). In addition, for the neoantigen derived from an indel mutation that triggered an in vitro immune response, Applicants found Ribo-seq reads that specifically support the translation of the frame-shifted nuORF in addition to the reads supporting the translation of the wild-type ORF (FIG. 4D). See Table for ORFs identified in melanoma patient used for Neoantigen prediction analysis.

In conclusion, Ribo-seq is a valuable tool for expanding and prioritizing potential cancer neoantigens. Ribo-seq predicted nuORFs are abundant and translated, and contribute antigens to MHC I presentation in healthy and cancer cells. Thus, nuORFs should be considered when selecting candidates for therapeutic neoantigen vaccines. In addition, given that thousands of nuORFs are differentially translated and mutated in cancer cells, their function needs to be investigated as they might generate additional targets for cancer therapy beyond neoantigen vaccines. Finally, Ribo-seq allows to determine whether cancer-specific variants are incorporated into translated proteins, in order to prioritize neoantigen selection for clinical intervention.

### Example 2: MHC-I Presentation of Unannotated Reading Frames

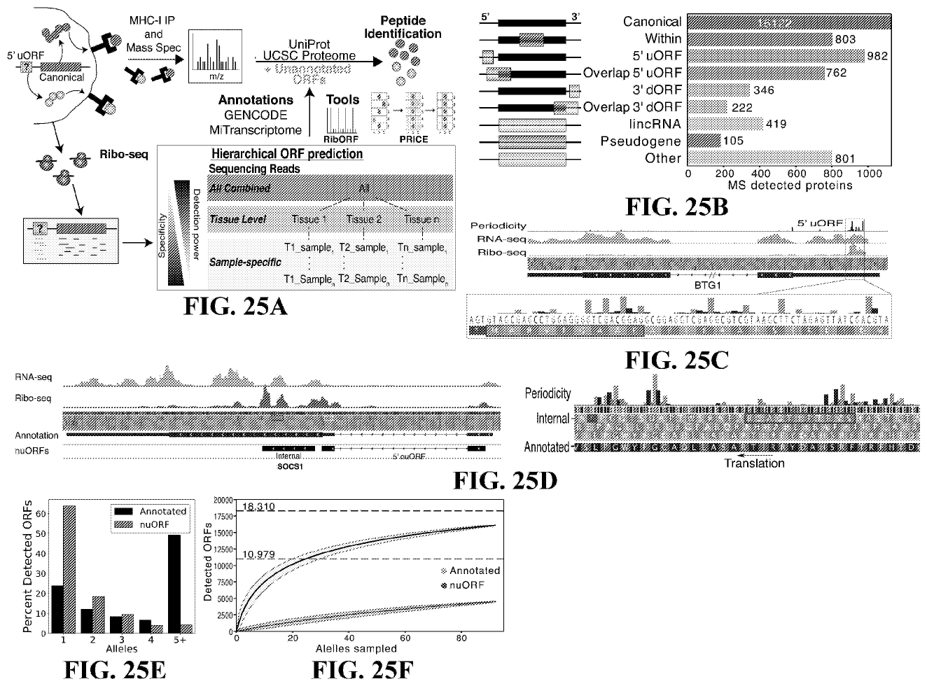

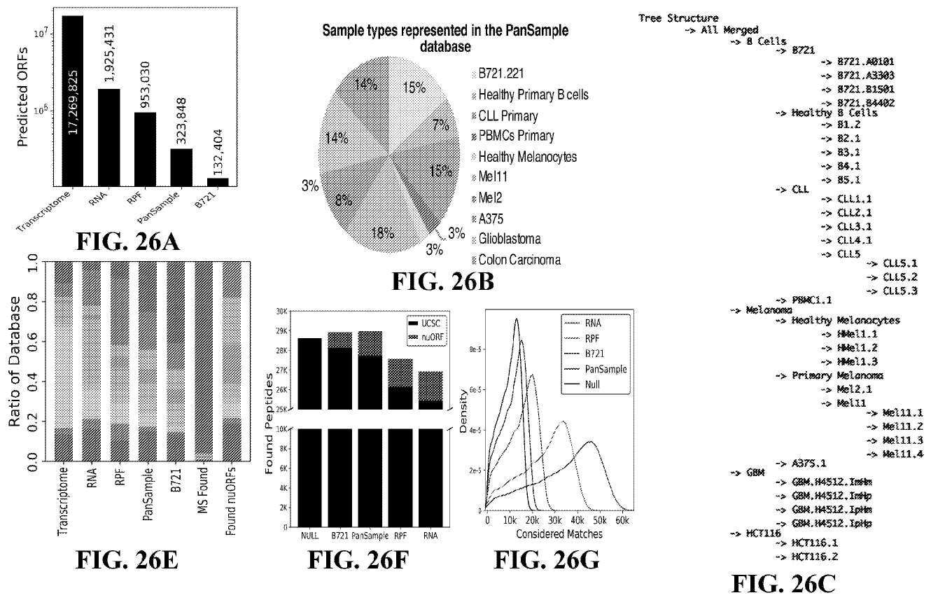

To determine whether novel unannotated open reading frames (nuORFs) contribute peptides for MHC I presentation, Applicants used a large collection of mono-allelic MHC class I immunopeptidome mass spectrometry (MS) data from 92 common HLA alleles expressed in B721.221 cells (Abelin et al., 2017). To identify the peptides detected by MS, the spectra are commonly searched against a database of annotated proteins. Therefore, proteins derived exclusively from nuORFs are not identified by MS. Including predicted nuORFs in the search space can improve MHC I immunopeptidome identification (FIG. 25a). All possible ORFs within transcripts in the GENCODE and MiTranscriptome annotations were identified. MiTranscriptome is based on the de novo transcriptome assembly of thousands of RNA-seq libraries from tumors, normal tissues and cell lines, complimenting GENCODE and allowing for nuORF prediction in healthy and tumor contexts (Frankish et al., 2019; Iyer et al., 2015). Assuming that an ORF must generate a protein that is at least 7 amino acids long for MHC I presentation, and that an ORF must start with NTG start codon, there were over 17 million possible ORFs across the annotated transcriptome (FIG. 26a). To narrow down the ORF candidates and to determine the effect of the database size on the MHC I MS spectra matching and peptide recovery, RNA-seq and Ribo-seq were performed on B721.221 cells and ORFs were selected that were supported by at least 2 reads to create the RNA-seq and Ribo-seq ORF databases. RibORF and PRICE were then used to predict translated ORFs based on the B721.221 Ribo-seq data, constructing the B721PredORF database (Erhard et al., 2018; Ji et al., 2015). Additionally, Ribo-seq was performed on 26 primary healthy and cancer samples and cell lines, including primary B cells, CLL cells, patient-derived primary glioblastoma, melanoma cell lines and healthy melanocytes, as well as additional colon carcinoma and melanoma cell lines (FIG. 26b). A hierarchical ORF prediction pipeline was developed to increase lowly transcribed ORF calling potential, while preserving tissue-specific ORFs (FIG. 26c). Using this pipeline, the PanSample database of predicted translated ORFs across samples was generated (FIG. 26a, e).

### Example 3: Database Size

To determine the effect of database size on peptide-spectra matching, the spectra of peptides bound to 15 common HLA alleles was searched against the 5 constructed databases. Across 15 alleles, the PanSample database produced the highest number of identified peptides, while the RNA-seq database had the least sensitivity (FIG. 26f). This effect can be attributed to the number of peptides considered for each spectrum, increasing the threshold for its identification (FIG. 26g). The PanSample database performed better than the B721PredORF database, demonstrating that combining shallowly sequenced Ribo-seq data across disparate samples improves the ORF prediction for an individual sample type. Additionally, this analysis demonstrates the value of using Ribo-seq to restrict the search space used for MS peptide assignment.

Searching the MHC I immunopeptidome MS spectra from 92 HLA alleles expressed in B721.221 cells against the PanSample database identified 9,454 peptides from 4,798 nuORFs of various categories, demonstrated that the nuORFs are translated and processed into peptides for MHC I presentation. Peptides from nuORFs represented 4.6% of the MHC I immunopeptidome, and 23% of all proteins identified (FIG. 25b, FIG. 26e). MHC I-displayed peptides were identified from 982 nuORFs within 5′ UTRs of coding transcripts (5′ uORFs) and from 803 nuORFs contained within but out-of-frame relative to an annotated ORF—categories of unannotated ORFs that are difficult to identify from RNA-seq data alone, but are more easily identifiable by Ribo-seq (FIG. 25c, d). Within some transcripts, like SOCS1, peptides from as many as three separate ORFs were detected in MHC class I immunopeptidome. For SOCS1, these included the annotated canonical protein, an internal out-of-frame nuORF and a 5′ overlapping uORF (FIG. 25d). The MHC class I detection of peptides from nuORFs was heavily dependent on the HLA type expressed in the cell line, such that most nuORFs were detected on only 1 or 2 alleles, while canonical ORFs were frequently identified across multiple HLA types (FIG. 25e). Although peptides were sampled from 92 HLA alleles, and nearly reached saturation for annotated protein detection, saturation of detected nuORFs was not approached (FIG. 25f). Across the diversity of HLA types, it is estimated that there are over 10,000 nuORFs to be translated and displayed on MHC class I.

### Example 4: NuORF-Derived Proteins

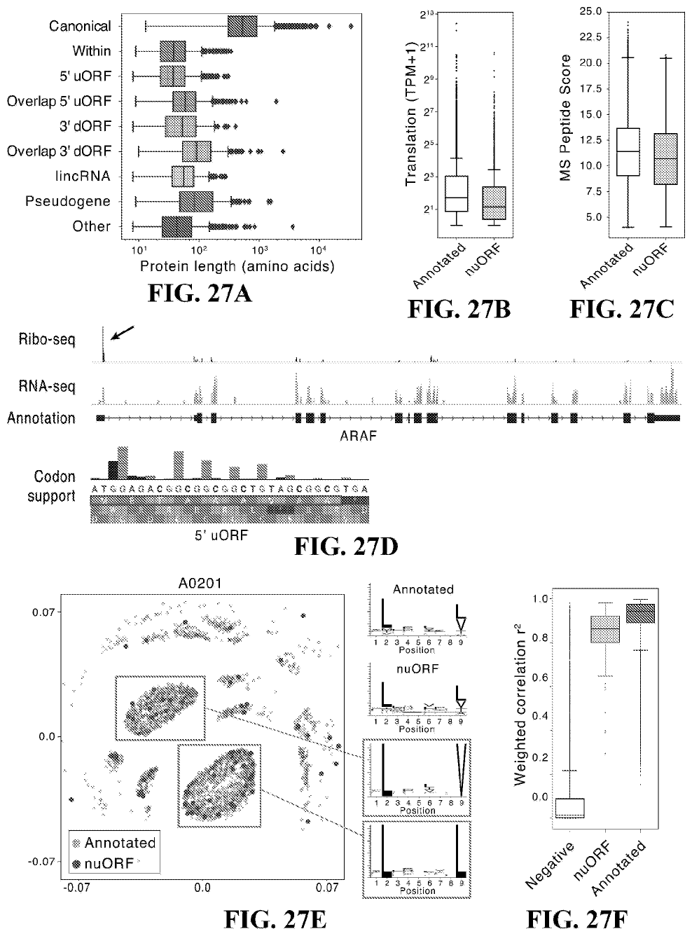

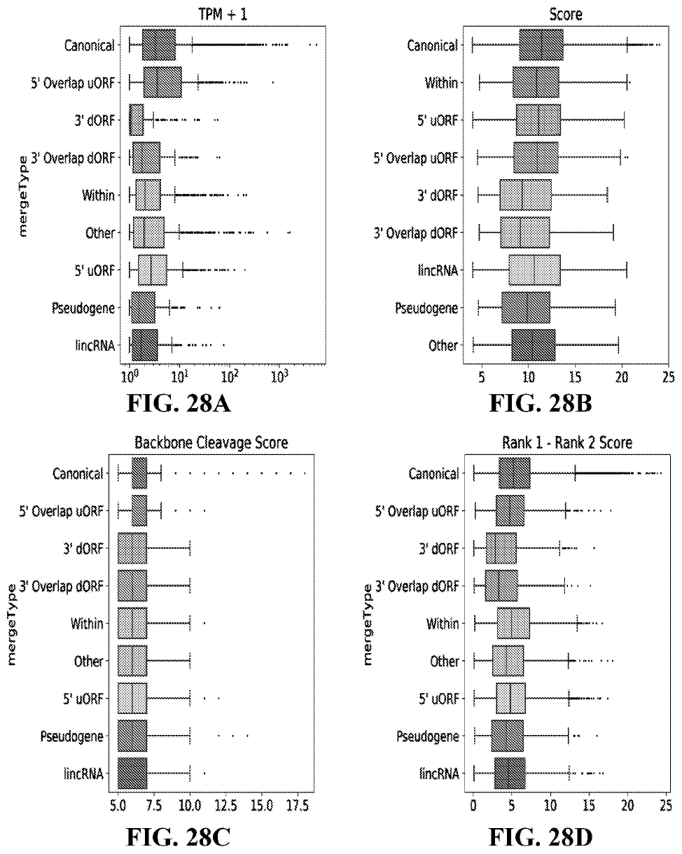

NuORF-derived proteins that contribute peptides to MHC I presentation are significantly shorter than annotated proteins (FIG. 27a). Additionally, some nuORF-derived proteins were exactly the length of their MHC I-bound antigens, suggesting that they do not require any additional processing to be presented on the MHC I. For example, a peptide was detected that corresponds to the entire translated 5′ uORF from the 5′ UTR of ARAF (FIG. 27d). The relative translation level (Ribo-seq vs. RNA-seq) of the 5′ uORF was higher than of the annotated ARAF protein (FIG. 27d). Overall, the translation levels of MHC I MS-detected nuORFs are comparable to annotated ORFs (FIG. 27b, FIG. 28a). Peptides from nuORFs and annotated ORFs are comparable in terms of MS detection score, false discovery rate and the backbone cleavage (FIG. 27c, FIG. 28b,c,d), further supporting that nuORFs generate high quality peptides for HLA presentation. In order for a peptide to be presented on MHC class I, the peptide sequence must correspond to the particular HLA type. Typically, MHC class I-bound peptides have several conserved positions called anchor residues. To compare nuORF-derived peptides to canonical ORF-derived peptides, non-metric multidimensional scaling analysis (NMDS) was performed on detected peptide sequences. nuORF derived peptides matched closely to peptides derived from annotated canonical proteins, suggesting that they are bona fide MHC I bound antigens (FIG. 27e,f).

### Example 5: Processing of NuORF Proteins and Peptides

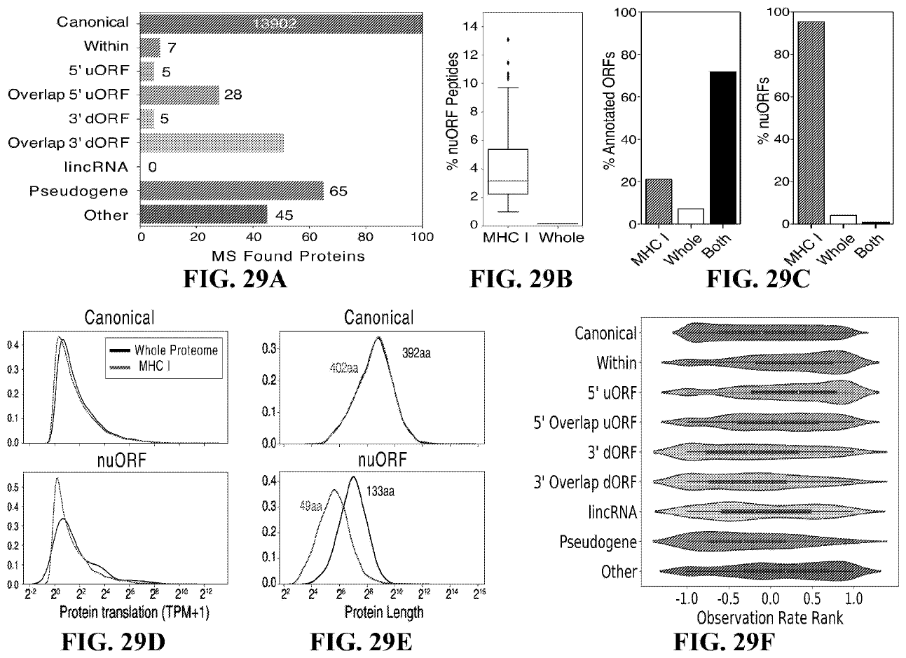

Whole proteome MS analysis on B721.221 cells identified 252 peptides derived from 202 nuORFs (FIG. 29a). Ribo-seq predicted nuORFs were under-represented in the whole proteome analyses, where they represented 0.2% of all identified peptides. This is strikingly different from the MHC class I immunopeptidome, where the median representation of nuORFs was 3.3% of found peptides (FIG. 29b). Only 7 internal nuORFs and 5 5′ uORFs were detected in whole proteome MS compared to 803 and 982 nuORFs, respectively, in MHC I immunopeptidome (FIGS. 29a and 25b). Additionally, while 72% of canonical proteins were observed in both the MHC class I immunopeptidome and in the whole proteome, only 0.5% of nuORFs were shared (FIG. 29c). While the absolute translation levels were similar between nuORFs detected on MHC I and in the whole proteome, the nuORFs detected on MHC I were much shorter than nuORFs detected in the whole proteome (FIG. 29d,e). ORF presentability was predicted based on allele specific normalized estimated MHC I binding affinity scores or length normalized in silico tryptic digest peptides. 5′ uORFs, 5′ overlapping uORFs, internal out-of-frame nuORFs detected on MHC I had significantly higher MHC I observation rates than detected canonical ORFs (FIG. 29f). These observations suggest potentially distinct mechanisms for processing peptides for MHC I presentation derived from shorter nuORFs versus longer canonical ORFs.

### Example 6: Analysis of NuORFs in Cancer Cells

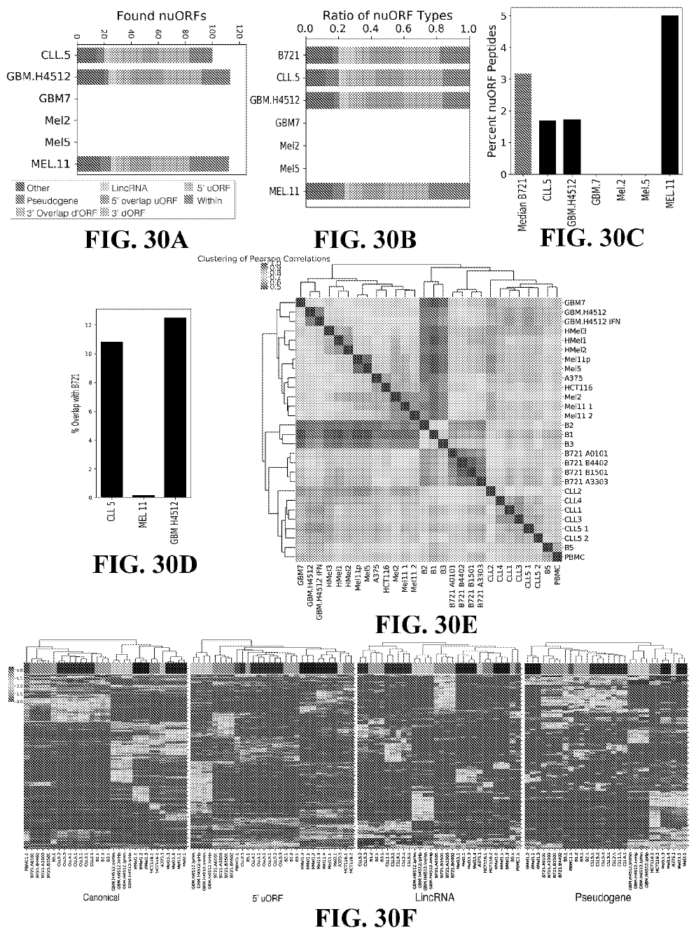

To determine if nuORF-derived proteins contribute antigens to MHC class I presentation in cancer cells, MHC I MS data from several melanoma, glioblastoma and CLL patient samples and tumor-derived cell lines were analyzed. Similar to B721.221 cells, peptides derived from hundreds of nuORF proteins in cancer cells were identified (FIG. 30a), confirming that nuORFs are translated and contribute peptides for MHC class I presentation regardless of sample type. The representation of various nuORF types was comparable between samples (FIG. 30b). Overall, nuORFs represented between 1.7% in CLL to 5% in melanoma (FIG. 30c). Detection of nuORF-derived peptides in the MHC I immunopeptidome depends heavily on the HLA type, despite differences in tissues of origin. 10.8% of peptides detected in the CLL sample and 12.5% in the glioblastoma sample were observed on the same alleles in B721.221 cells (FIG. 30d).

To determine if nuORFs are differentially translated across cancer and healthy tissues, the absolute translation levels were calculated based on the Ribo-seq data. Overall translation correlated well between samples of similar origins (FIG. 30e). Therefore, Applicants compared the absolute translation levels of nuORFs were compared in cancer samples and healthy tissue samples (CLL vs. B cells, and melanoma vs. melanocytes), and in other unrelated cancer and healthy samples. Depending on the cancer type and the quality of Ribo-seq data, tens to hundreds of nuORFs were identified that are differentially translated in cancer- and patient-specific manner (FIG. 30f). Thus, despite the small number of samples used for this analysis, these preliminary findings demonstrate that Ribo-seq is a useful tool to identify nuORFs translated in a cancer-specific manner, contributing additional neoantigens.

### Example 7: Cancer-Specific Somatic Mutations in NuORFs are Neoantigens

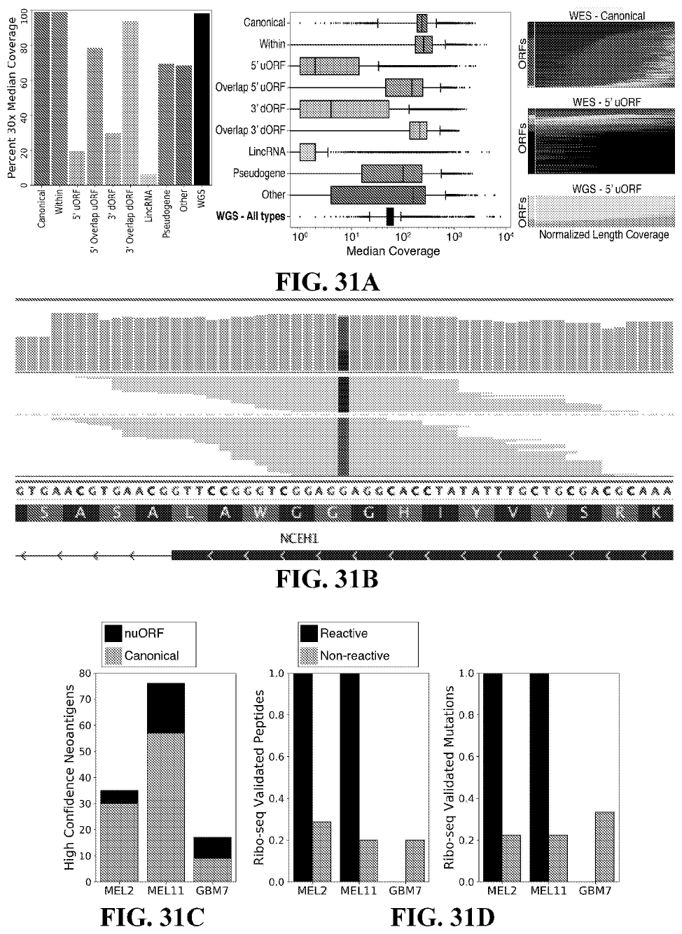

To determine if cancer-specific somatic mutations in nuORFs are a source of neoantigens in cancer, it was first determined whether WES provides sufficient coverage to capture somatic mutations across various categories of nuORF. While over 99% of canonical ORFs had over 30× median coverage, the recommended level for variant calling, the coverage across nuORFs varied greatly, and only 19.5% of 5′ uORFs and 6% of lincRNAs had similar coverage (FIG. 31a). In comparison, whole genome sequencing (WGS) provides much more uniform coverage across annotated ORFs and nuORFs, with at least 30× median coverage of over 98% of ORFs across all ORF types (FIG. 31a). To compare mutation rates across annotated ORFs and nuORFs, somatic variants from 1903 patient datasets publicly available through the International Cancer Genome Consortium (ICGC) were mapped. WGS was performed on a cell line derived from a melanoma patient that has participated in a neoantigen vaccine clinical trial and matched PBMCs (Ott et al., 2017) and it was confirmed that the melanoma patient-derived cell line closely recapitulates the original patient tumor (Table 4).

TABLE 4
Melanoma cell line recapitulates tumor

| | Tumor-WES | Cell line-WGS | Common |
|---|---|---|---|
| SNVs | 5132 | 358741 | 4982 (97%) |
| Indels | 5132 | 5132 | 40 (78%) |

In order to select the most likely neoantigens that could be derived from somatic variants in nuORFs and canonical ORFs, neoantigens were prioritized that Ribo-seq indicated were translated and had reasonable predicted MHC class I binding affinity (Hoof et al., 2009) (FIG. 31c). Incorporating Ribo-seq data allowed selection of cancer-specific somatic variants that are incorporated into translated proteins, increasing the likelihood of them being presented on MHC class I. To identify cancer-specific variants that are incorporated into translated ORFs, a computational pipeline was developed to obtain the number of Ribo-seq reads supporting the translation of the mutant and wild-type alleles for both single nucleotide variants (SNVs) and insertions and deletions (indels). This pipeline and MHC class I binding affinity prediction by netMHCpan was used to identify a set of potential neoantigens from one glioblastoma and two melanoma cell lines derived from patients that have previously participated in neoantigen vaccine clinical trials (Keskin et al., 2019; Ott et al., 2017). On average, across three patients, 29% (14% in Mel2, 25% in Mel11, 47% in GBM7) of potential neoantigens were derived from nuORFs, indicating that nuORFs are a viable source of additional neoantigens in cancer (FIG. 31d). The variants selected for the neoantigen vaccines in these melanoma and glioblastoma patients were re-analyzed. In melanoma samples, the variants that successfully triggered an immune response in T cell assays in vitro had Ribo-seq translation and variant read coverage support versus the variants that failed the in vitro validation (FIG. 31e). In the glioblastoma sample, the variants confirmed in the in vitro assays did not have sufficient Ribo-seq support, highlighting the caveats of using a patient-derived cell line as a proxy for the original tumor. Finally, for the neoantigen derived from an indel mutation in RALGAPB that triggered an in vitro immune response in melanoma patient 11, Ribo-seq reads that specifically support the translation of the frame-shifted nuORF were found in addition to the reads supporting the translation of the wild-type ORF (Table 4).

### ICGC Analysis

Genome-wide somatic mutations across 1903 cancer samples from ICGC to all ORFs were incorporated within the PanSample database. Across all cancers, expected mutated genes, such as KRAS, p53, CDKN2A, etc. were found.

Multiple 5′ uORFs were found within the BCL2 5′ UTR. BCL2 is known to be heavily post-transcriptionally regulated, with a highly conserved 5′ UTR, and an IRES site allowing for cap-independent translation. However, it has been underappreciated so far that 5′ uORFs within the 5′ UTR of BCL2 are actually translated into peptides, which can be detect by MS in the MHC I immunopeptidome. Specific residues within these translated 5′ uORFs are frequently mutated, suggesting an additional functional role.

A 3′ dORF were found in UNG that is frequently mutated in prostate adenocarcinomas (PRAD). Interestingly, across all patients, the mutation commonly occurs at the same genomic position.

Multiple uORFs were found in the 5′ UTR of TMSB4X, several of them harboring multiple mutations. TMSB4× is a gene frequently upregulated in cancer and the vast majority of mutations in TMSBX4 gene fall within the 5′ UTR rather than the annotated coding sequence.

A 5′ uORF was found in the IGLL5 gene. Within ICGC, the mutations occur in malignant lymphomas. Interestingly, IGLL5 gene has been shown to be the most frequently mutated gene in 13q-deficient CLL (Kasar et al., 2015).

A 3′ dORF was found in LCP1 at the same position where there is a recurrent mutation in prostate adenocarcinomas.

A recurring PDE10A mutation was found in an iORF. Since it is an iORF, it overlaps with canonical. However, the mutation in the overlapping canonical protein does not result in amino acid change, whereas the mutation in the iORF does induce an amino acid change, indicating that it could be functional.

### REFERENCES

Abelin, J. G., Keskin, D. B., Sarkizova, S., Hartigan, C. R., Zhang, W., Sidney, J., Stevens, J., Lane, W., Zhang, G. L., Eisenhaure, T. M., et al. (2017). Mass Spectrometry Profiling of HLA-Associated Peptidomes in Mono-allelic Cells Enables More Accurate Epitope Prediction. Immunity 46, 315-326.
Anderson, D. M., Anderson, K. M., Chang, C. L., Makarewich, C. A., Nelson, B. R., McAnally, J. R., Kasaragod, P., Shelton, J. M., Liou, J., Bassel-Duby, R., et al. (2015). A micropeptide encoded by a putative long noncoding RNA regulates muscle performance. Cell 160, 595-606.
Budamgunta, H., Olexiouk, V., Luyten, W., Schildermans, K., Maes, E., Boonen, K., Menschaert, G., and Baggerman, G. (2018). Comprehensive Peptide Analysis of Mouse Brain Striatum Identifies Novel sORF-Encoded Polypeptides. Proteomics, e1700218.
Erhard, F., Halenius, A., Zimmermann, C., L'Hernault, A., Kowalewski, D. J., Weekes, M. P., Stevanovic, S., Zimmer, R., and Dolken, L. (2018). Improved Ribo-seq enables identification of cryptic translation events. Nat Methods.
Fields, A. P., Rodriguez, E. H., Jovanovic, M., Stern-Ginossar, N., Haas, B. J., Mertins, P., Raychowdhury, R., Hacohen, N., Carr, S. A., Ingolia, N. T., et al. (2015). A Regression-Based Analysis of Ribosome-Profiling Data Reveals a Conserved Complexity to Mammalian Translation. Mol Cell 60, 816-827.
Frankish, A., Diekhans, M., Ferreira, A. M., Johnson, R., Jungreis, I., Loveland, J., Mudge, J. M., Sisu, C., Wright, J., Armstrong, J., et al. (2019). GENCODE reference annotation for the human and mouse genomes. Nucleic Acids Res 47, D766-D773.
Hoof, I., Peters, B., Sidney, J., Pedersen, L. E., Sette, A., Lund, O., Buus, S., and Nielsen, M. (2009). NetMHCpan, a method for MHC class I binding prediction beyond humans.
Immunogenetics 61, 1-13.
Iyer, M. K., Niknafs, Y. S., Malik, R., Singhal, U., Sahu, A., Hosono, Y., Barrette, T. R., Prensner, J. R., Evans, J. R., Zhao, S., et al. (2015). The landscape of long noncoding RNAs in the human transcriptome. Nat Genet 47, 199-208.
Ji, Z., Song, R., Regev, A., and Struhl, K. (2015). Many lncRNAs, 5′UTRs, and pseudogenes are translated and some are likely to express functional proteins. eLife 4.
Kasar, S., Kim, J., Improgo, R., Tiao, G., Polak, P., Haradhvala, N., Lawrence, M. S., Kiezun, A., Fernandes, S. M., Bahl, S., et al. (2015). Whole-genome sequencing reveals activation-induced cytidine deaminase signatures during indolent chronic lymphocytic leukaemia evolution. Nat Commun 6, 8866.
Keskin, D. B., Anandappa, A. J., Sun, J., Tirosh, I., Mathewson, N. D., Li, S., Oliveira, G., Giobbie-Hurder, A., Felt, K., Gjini, E., et al. (2019). Neoantigen vaccine generates intratumoral T cell responses in phase Ib glioblastoma trial. Nature 565, 234-239.
Makarewich, C. A., and Olson, E. N. (2017). Mining for Micropeptides. Trends Cell Biol.
Ott, P. A., Hu, Z., Keskin, D. B., Shukla, S. A., Sun, J., Bozym, D. J., Zhang, W., Luoma, A., Giobbie-Hurder, A., Peter, L., et al. (2017). An immunogenic personal neoantigen vaccine for patients with melanoma. Nature.
Sahin, U., Derhovanessian, E., Miller, M., Kloke, B. P., Simon, P., Lower, M., Bukur, V., Tadmor, A. D., Luxemburger, U., Schrors, B., et al. (2017). Personalized RNA mutanome vaccines mobilize poly-specific therapeutic immunity against cancer. Nature 547, 222-226.
Sendoel, A., Dunn, J. G., Rodriguez, E. H., Naik, S., Gomez, N. C., Hurwitz, B., Levorse, J., Dill, B. D., Schramek, D., Molina, H., et al. (2017). Translation from unconventional 5′ start sites drives tumour initiation. Nature 541, 494-499.

### Example 8: Methods

Applicant's methods of predicting highly relevant, immunongenic neoantigens that can be used in a pharmaceutical vaccine composition.

### Ribosomal Profiling

Ribosomal profiling was performed according to the Illumina protocol (TruSeq Ribo Profile—RPHMR12126, discontinued), with some modifications. In brief, for adherent cell lines (melanoma, primary melanocytes, HCT116, A375), culture media was removed, cells were washed with ice-cold PBS containing cycloheximide (0.1 mg/ml) and lysed in the Lysis Buffer according to the Illumina protocol. Briefly, mammalian lysis buffer was added to cells, and cells were scraped extensively (and pipetted to ensure completely lysis). Lysed cells were transferred, to a microcentrifuge tube and placed on ice for 10 minutes (and inverted periodically to mix). Lysed cells were then centrifuged at 20,000×g for 10 minutes at 2 to 8 degrees Celcius. Following centrifugation, the supernatant was transferred to a new microcentrifuge tube on ice. The recovered lysate was diluted 1:10 in nuclease free water. A260 readings were recorded using a spectrophotometer and the concentration was calculated using the formula: (A₂₆₀ cell lysate-A₂₆₀ mammalian cell buffer)×10 dilution factor=A₂₆₀/ml. When preparing ribosome footprints, this number was used to calculate the amount of TruSeq ribo profile nuclease for generating RPF. Two samples were created from the cell lysate: (A) for preparing total RNA library and (B) for preparing ribosome footprints. 10% SDS was added to sample (A) and mixed using a pipette.

For suspension cell lines and primary blood samples, cells were spun 1000 rpm for 5 minutes, washed once with ice-cold PBS containing cycloheximide (0.1 mg/ml) and lysed in the Lysis Buffer. To perform Ribo-seq on small samples, such as primary B cells and melanocytes, cells were lysed in 200 ul of lysis buffer, such that the entire lysate could be used in the library preparation.

TruSeq Ribo Profile Nuclease was added to sample (B), and the mixture was shaken at room temperature for 45 minutes. SUPERase*In RNase inhibitor was added to the mixture to stop the reactions.

The ribosomes containing ribosome-protected mRNA fragments (RPFs) were enriched using MicroSpin S-400 columns (GE Healthcare, catalog #27-5140-01). Two columns per sample were used, with 100 ul of digested RPF loaded into each column, such that the entire lysate could be processed.

Ribo-zero rRNA Removal Kit (Illumina, MRZH11124, discontinued) was used to deplete rRNA from RPFs. After rRNA removal, the RPF were taken directly to gel electrophoresis without quantification. The entire RPF sample (20 ul) was loaded on a 15% urea-polyacrylamide gel. The samples were eluted from the gel overnight at 4° C. Subsequently, end repair, adapter ligation and reverse transcription were carried out according to the Illumina protocol.

For cDNA gel purification, the entire reverse transcription reaction was loaded on a 10% urea-polyacrylamide gel, split over 3-4 wells. The samples were eluted from the gel overnight at room temperature. Subsequently, RPFs were circularized and 5 ul of circDNA was used for library amplification. The number of amplification cycles was determined based on the observed sample quality and expected yield, but usually ranged between 8 and 10 cycles. Following amplification, the library was gel-purified using 4% E-Gel EX Agarose Gel (ThermoFisher G401004) and Zymoclean Gel DNA Recovery Kit (Zymo Research D4007), with 4 volumes of ADB buffer to accommodate 4% agarose gel. Library cleanup with agarose gel took less than 1 hour, in contrast to the original protocol which used polyacrylamide gel and could take up to one extra day.

The resulting libraries were analyzed for quality using Agilent Bioanalyzer 2100 and sequenced on Illumina NextSeq platform.

### Hierarchical Prediction of Translated Open Reading Frames Across Tissues

To process RPF sequencing reads, Illumina adapters were removed using fastx_clipper from the FASTX-Toolkit. Ribosomal RNA and tRNA were removed using bowtie. Remaining reads were aligned to the genome and transcriptome using STAR (—alignIntronMin 20—alignIntronMax 100000—outFilterMismatchNmax 1—outFilterType BySJout—outFilterMismatchNoverLmax 0.04—twopassMode Basic).

To determine the RPF library quality, trinucleotide codon periodicity was plotted using RibORF readDist script against annotated protein-coding ORFs. Only samples that showed clear trinucleotide periodicity were used for subsequent ORF predictions. Hierarchical ORF predictions were done using RibORF and PRICE (Erhard et al., 2018; Ji et al., 2015). In order to maximize translated ORF detection and prevent noise from overlapping ORFs expressed in different tissues, Applicants performed hierarchical ORF prediction.

RibORF

Only read lengths that showed clear trinucleotide periodicity were used for ORF predictions using RibORF. RibORF offsetCorrect script was used to correct the RPF offsets for each read length. Corrected SAM files were subsequently used for ORF predictions. Corrected SAM files across samples were combined at each node for ORF predictions.

For the transcriptome reference, GENCODE v26lift37 transcriptome annotation was combined with transcripts annotated as tstatus “unannotated” from MiTranscriptome annotation (Iyer et al., 2015). From this custom transcriptome reference, all possible ORFs with NTG start codons and TAA/TGA/TAG stop codons were identified using Rp-Bp prepare-rpbp-genome script (Malone et al., 2017).

After running RibORF, only ORFs with the score above 0.7 were retained. From a set of ORFs on the same transcript that share a common stop codon, the longest ORF was selected. For the hierarchical ORF prediction, Applicants kept all predicted ORFs with at least 2 reads in frame and RibORF score greater than 0.7 from the combined All Merged node. Applicants then added node-specific ORFs that had either 2 reads and 0.9 score, or 250 reads and 0.7 score. PRICE

PRICE pipeline was run on unprocessed fastq.gz files of the samples that had clear tri-nucleotide periodicity. The pipeline took care of adapter trimming, rRNA and tRNA removal, offset correction and ORF prediction. The same reference transcriptome was used for PRICE as for RibORF. Unique .cit files were generated for each sample. gedi MergeClT was used to merge samples by tissue type at each node. gedi Price-fdr 1 was used to predicted translated ORFs.

### Generating the PanSample and the B721.221 ORF Databases

Fasta files of ORFs predicted across tissues by RibORF and PRICE were combined, and those ORFs entirely contained within other predicted ORFs at the protein level were removed to reduce redundancies in order to generate the PanSample database. B721.221 ORF database was constructed in a similar manner, except it contains all ORFs predicted by PRICE and RibORF exclusively in B721.221 cells. Predicted ORFs over 21 nucleotides long were retained for the downstream analysis.

### Whole Genome Sequencing and Analysis

PCR-free whole genome sequencing (WGS) was performed on cancer cells and matched healthy PBMCs at the Broad Genomics Platform. Cancer-specific variants were identified using GATK Best Practices.

### Variant Analysis, Read Coverage, and Neoantigen Predictions

Python scripts and Jupiter notebooks used in the analysis will be deposited in GitHub. In brief, to derive ORFs containing cancer-specific variants identified by WGS, variants that were found within the reference transcripts used in the study were selected using bedtools intersect of the BED12 file of transcripts with the vcf file of variants. Variants were then incorporated into the transcript sequences, and ORFs were re-derived based on the predicted start codon in the PanSample database and the first in-frame stop codon.

To obtain RNA-seq and Ribo-seq read coverage and nucleotide identity at the single nucleotide variant (SNV) sites, pysam pileup was used. To obtain read coverage of indels, bowtie-m 1-v0 was used to align raw sequencing reads (after adapter trimming) to a custom Fasta reference that included matched wild-type and indel-containing transcripts. No multimapping reads or mismatches were allowed, such that only variant- or wild-type supporting reads were retained.

To obtain potential neoantigens from the mutated variants, all possible 9- and 10-amino acid long peptides were derived from wild-type and variant-containing proteins in the PanSample database. Peptides unique to the variant-containing proteins were retained as potential neoantigens. NetMHCpan v4.0 was used to predict neoantigen binding affinities to HLA alleles. Indels were visualized in IGV to identify in-frame Ribo-seq reads supporting the translation of indel-generated frame-shifted ORFs and wild-type ORFs.

### Absolute Translation Estimation

To calculate the absolute translation levels of nuORFs, only reads aligned in-frame with the translated codons were used. Reads in one of the three possible frames were calculated using bedtools coverage. Reads in the translated frame only were used for TPM calculations.

Identification of Tissue-Specific or Tissue-Enriched nuORFs

For the highly variable ORFs, all ORFs in the PanSample database were binned by mean log₂(TPM+1) across all samples to give approximately the same number of ORFs per bin. Highly variable ORFs were defined as ORFs with variance greater than 2 standard deviations from the mean variance within each bin.

HLA-peptide immunoprecipitation, sequencing by tandem mass spectrometry, interpretation, and whole proteome analysis and interpretation as previously described in Abelin et al., 2017.

### Example 9—Thousands of Novel Unannotated Proteins Expand the MHC I Immunopeptidome in Cancer

The major histocompatibility complex class I (MHC I) immunopeptidome consists of thousands of short 8-12 amino acid peptide antigens displayed on the cell surface by MHC I molecules. “Non-self” antigens presented by MHC molecules are recognized by CD8 T cells that mount an immune response. This defense mechanism can be exploited to target the immune system against cancer cells, which display cancer-specific antigens (neoantigens) on MHC I (1). Patients immunized with neoantigen-based vaccines display expanded neoantigen-specific T cells, suggesting that this could be a promising therapeutic avenue (2-5).

Neoantigens are currently predicted based on the detection of cancer-specific somatic mutations in annotated protein-coding regions by whole exome sequencing (WES) and RNA-seq (2-5). This approach often falls short for patients with few somatic mutations, generating few actionable neoantigens (6). Several lines of evidence suggest that the potential sources of neoantigens are more varied. First, immune responses have been detected against antigens derived from retained introns, alternative open reading frames (ORFs) within coding genes, and antisense transcripts (7-9). Additionally, while the MHC I immunopeptidome mainly consists of peptides derived from homeostatic protein turnover (10, 11), peptides can also be sourced from alternative precursors, such as defective ribosomal products (DRiPs), and presumably “non-coding” regions of the genome (12-14). Finally, ribosome profiling (Ribo-seq), which assays mRNA translation by capturing and sequencing ribosome-protected mRNA fragments (RPFs) (15), has detected a plethora of translated novel unannotated open reading frames (nuORFs). These nuORFs are derived from the 5′ and 3′ untranslated regions (UTRs), overlapping yet out-of-frame alternative ORFs in annotated protein-coding genes, long non-coding RNAs (lncRNAs), pseudogenes and other transcripts currently annotated as non-protein coding (16-18). Ribo-seq analysis of HEK293T, HeLa-S3, and K562 cell lines and of human fibroblasts infected with HSV-1 and HCMV has identified translated nuORFs that contribute peptides to the MHC I immunopeptidome, suggesting that nuORFs could also have an immunological function (19, 20). However, a global understanding about the extent to which nuORFs contribute to the immunopeptidomes of healthy and cancer tissues, as well as the diversity and tissue specificity of nuORFs is still lacking.

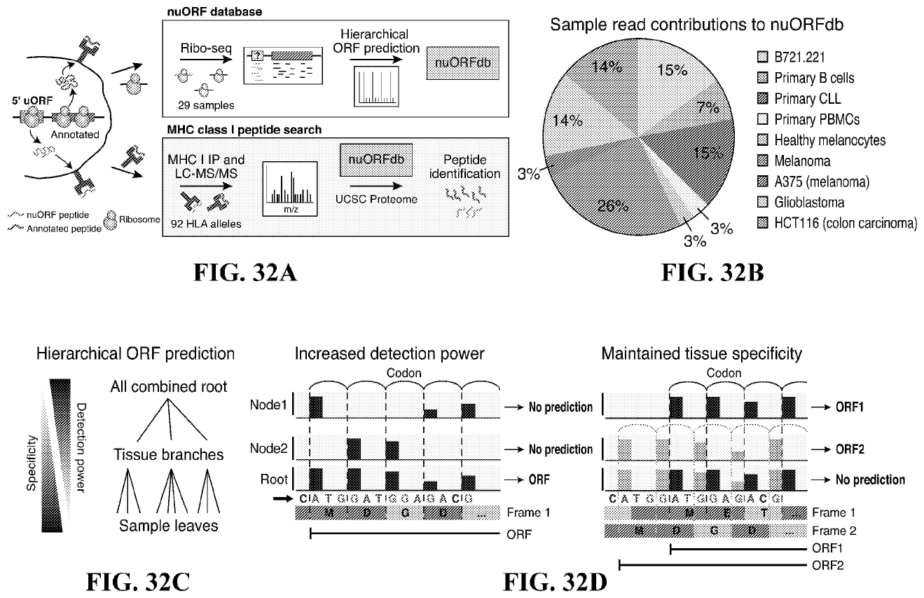

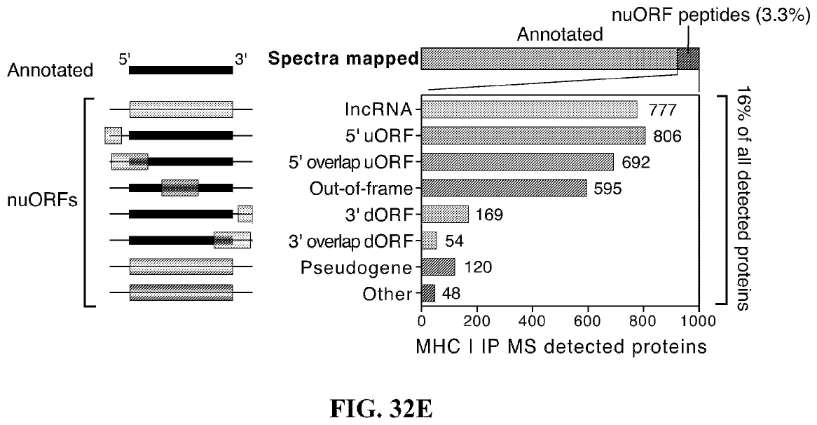

Applicants hypothesized that cancer-associated processes could lead to nuORFs that are either mutated or exhibit tumor-specific expression and thus could serve as sources of neoantigens. To systematically evaluate the contribution of nuORFs to the MHC I immunopeptidome, we: (1) identified translated nuORFs using Ribo-seq, (2) built an ORF database appending nuORFs detected by Ribo-seq to known annotations, and (3) used this updated database to search for presented nuORFs in MHC I immunopeptidome mass spectrometry (MS) data (FIG. 32A). Because MS/MS spectra are traditionally searched against a sequence database of annotated proteins, any presented peptides derived exclusively from nuORFs (which are, by definition, not in the standard annotated protein database) will not be identified by conventional search strategies. Therefore, Applicants reasoned that including Ribo-seq-detected nuORFs in the search space can improve MHC I immunopeptidome identification (FIG. 32A).

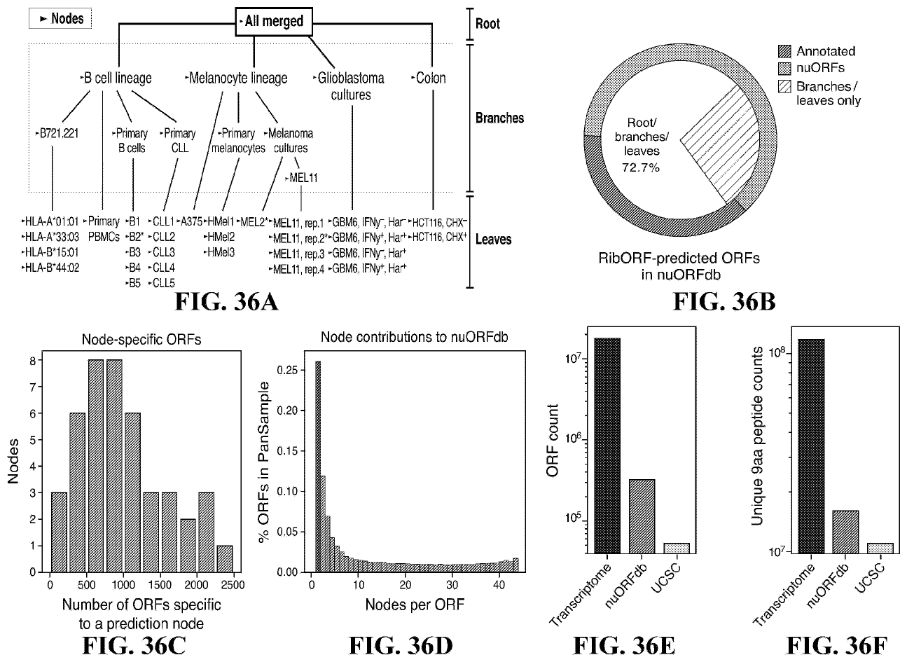

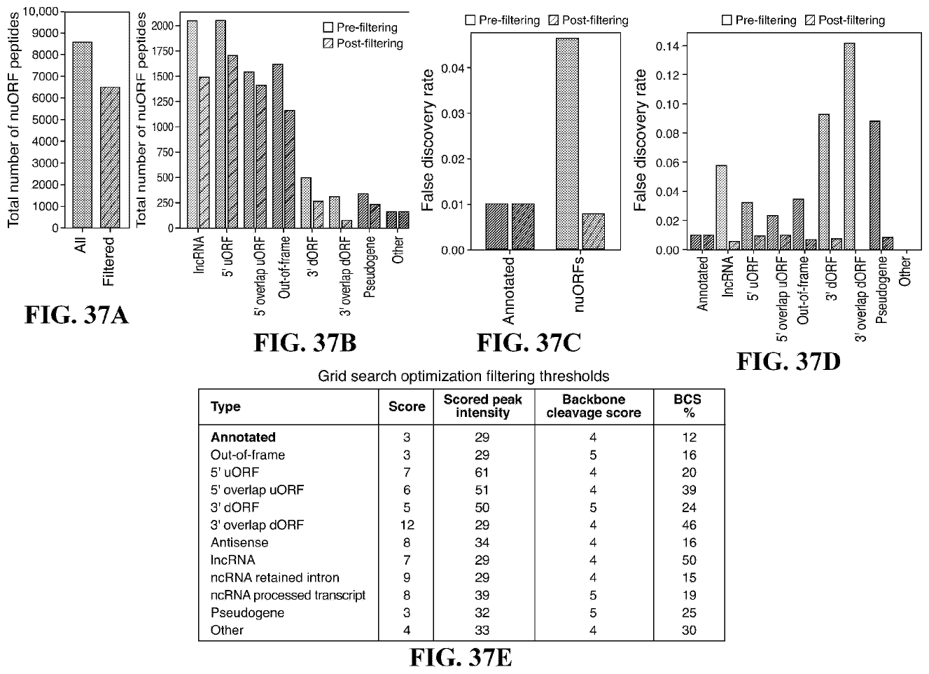

To this end, Applicants collected Ribo-seq data from 29 primary healthy and cancer samples and cell lines. These included primary normal and chronic lymphocytic leukemia (CLL) B cells, patient-derived primary glioblastoma (GBM) and melanoma cell cultures, primary healthy melanocytes, as well as established colon carcinoma and melanoma cell lines. These also included B721.221 cells, the parental cell line previously used to generate 92 single HLA allele-expressing lines from which Applicants collected mono-allelic MHC I immunopeptidome data (10, 11) (FIG. 32B, FIG. 36A). Applicants developed a hierarchical ORF prediction pipeline, where ORF predictions were carried out at multiple prediction nodes, consisting of each sample (leaf), tissue (branch) and across all samples combined (root) (FIG. 32C, FIG. 36A, SOM). This approach aggregated signal across the Ribo-seq dataset to predict lowly translated ORFs, while maintaining sensitivity for tissue-specific, overlapping ORFs (FIG. 32D). Applicants predicted translated ORFs within transcripts annotated in GENCODE (Frankish et al. 2019) and MiTranscriptome, which contains de novo assembled transcripts from thousands of RNA-seq libraries from tumors, normal tissues and cell lines and thus might be particularly useful to identify cancer-specific nuORFs (Iyer et al. 2015). Thus, Applicants generated nuORFdb v1.0, containing 86,421 annotated and 237,427 nuORFs (323,848 ORFs in total). NuORFdb had ˜50-fold less ORFs than the ˜17 million ORFs obtained from the combined transcripts in the GENCODE and MiTranscriptome annotations (FIG. 36E). Compared to the annotated proteome (UCSC), nuORFdb had only 1.46-fold more HLA class-I candidate peptides of length 9, making it very practical for routine use in immunopeptidomics studies (FIG. 36F).

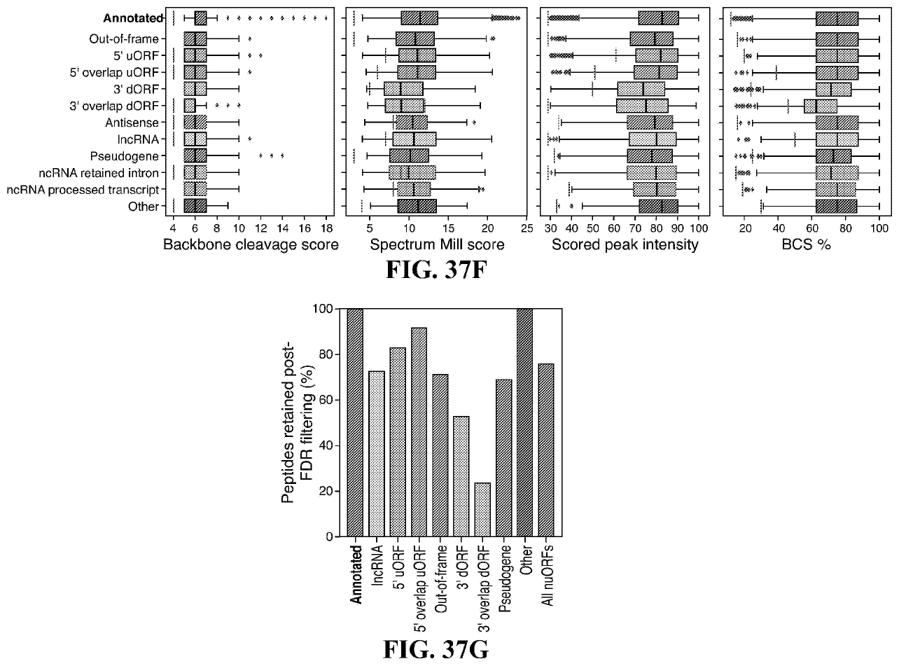

Searching the MHC I immunopeptidome MS/MS spectra from 92 HLA alleles expressed in B721.221 cells (11) against nuORFdb with stringent FDR filtering (FIG. 37A-37G), Applicants identified 6,501 high confidence (FDR<1%) peptides from 3,261 nuORFs, across various nuORF types (SOM, FIG. 32E, FIG. 37A-37G), contributing 3.3% of peptides to the WIC I immunopeptidome (FIG. 32E).

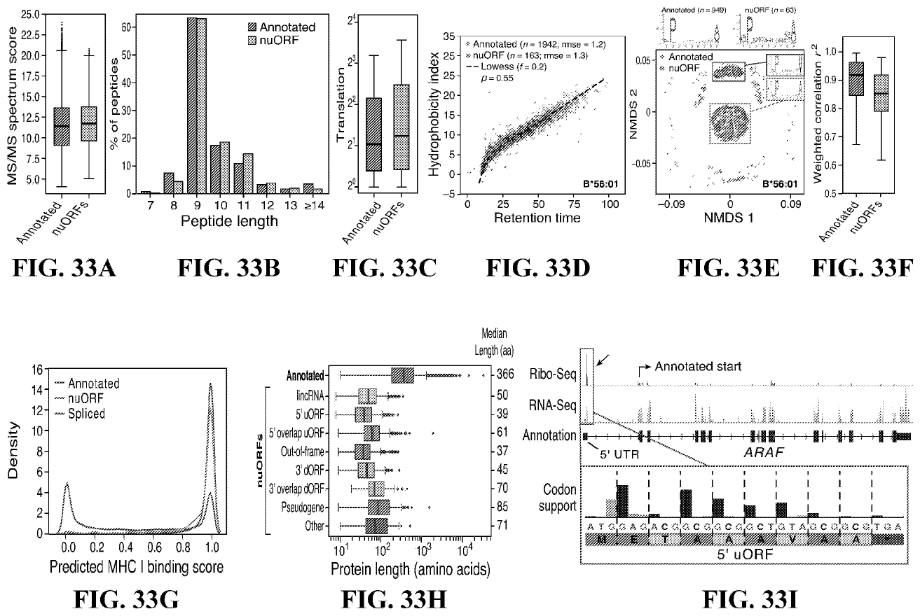

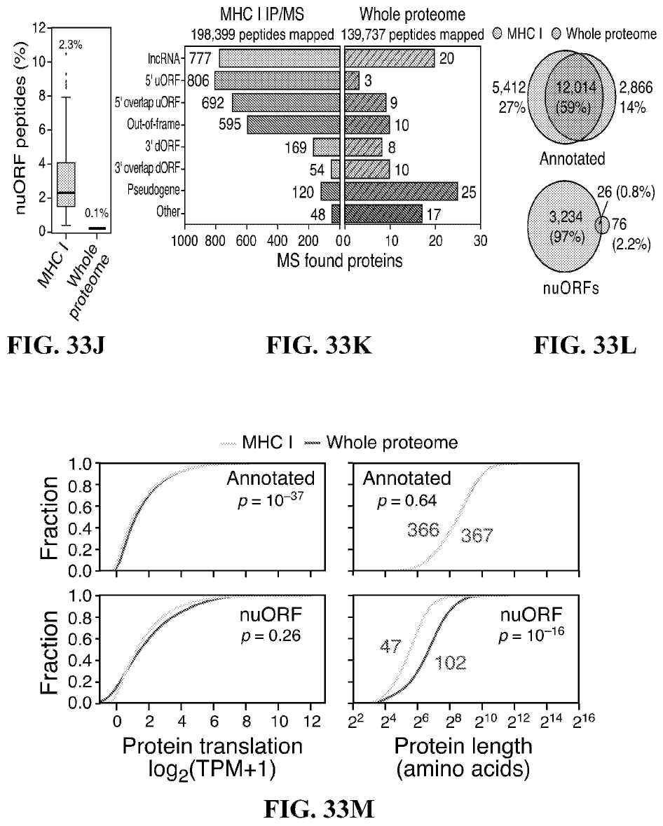

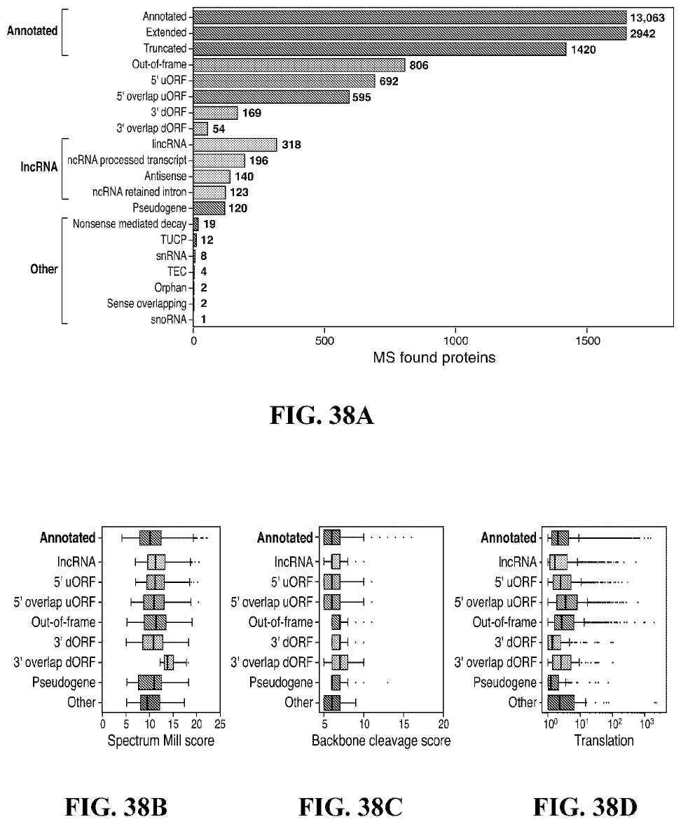

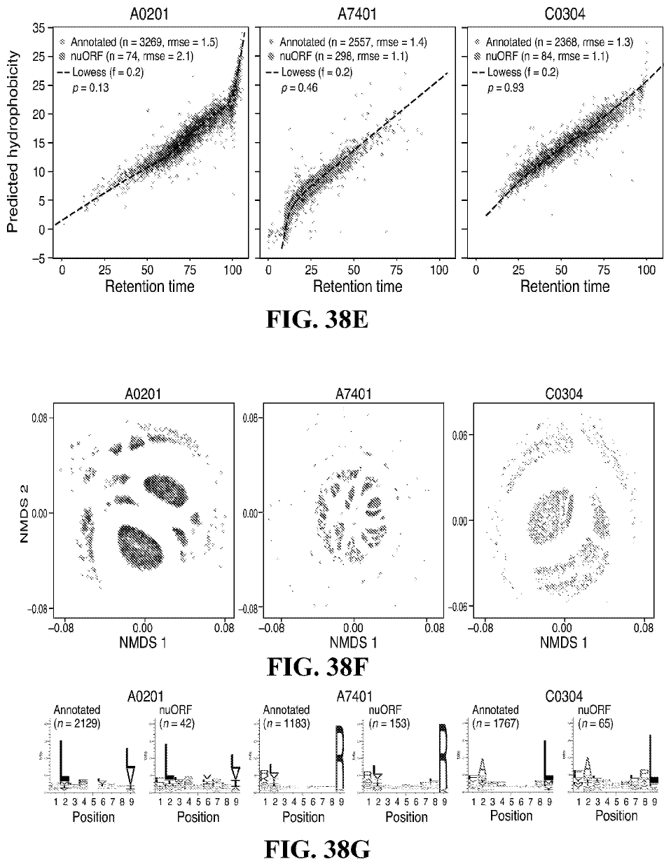

Several lines of evidence revealed the MS/MS-identified nuORF peptides to be of comparable quality and characteristics to peptides from annotated ORFs (“annotated peptides”). First, nuORF and annotated MS/MS-detected peptides had similar Spectrum Mill MS/MS identification scores (11.7 nuORF vs. 11.4 annotated mean scores, 95% CI: 0.27-0.43), median peptide length (9AA), and translation levels (1.7 nuORF vs. 1.6 annotated mean log₂TPM, 95% CI: 0.09-0.19) (FIG. 33A-33C, FIG. 38B-38D). Moreover, chromatographic retention times for nuORF peptides correlated as well with predicted hydrophobicity indices as they did for annotated peptides (p=0.55, rank-sum test) (FIG. 33D, FIG. 38E) (21, 22). Finally, two-dimensional projection of pairwise distances amongst detected peptide sequences per allele showed that nuORF-derived peptides matched closely to peptides derived from annotated proteins (FIG. 33E, FIG. 38F, 38G) with a strong agreement in peptide sequences across all alleles (Pearson r²=0.85 nuORFs, r²=0.92 annotated) (FIG. 33F).

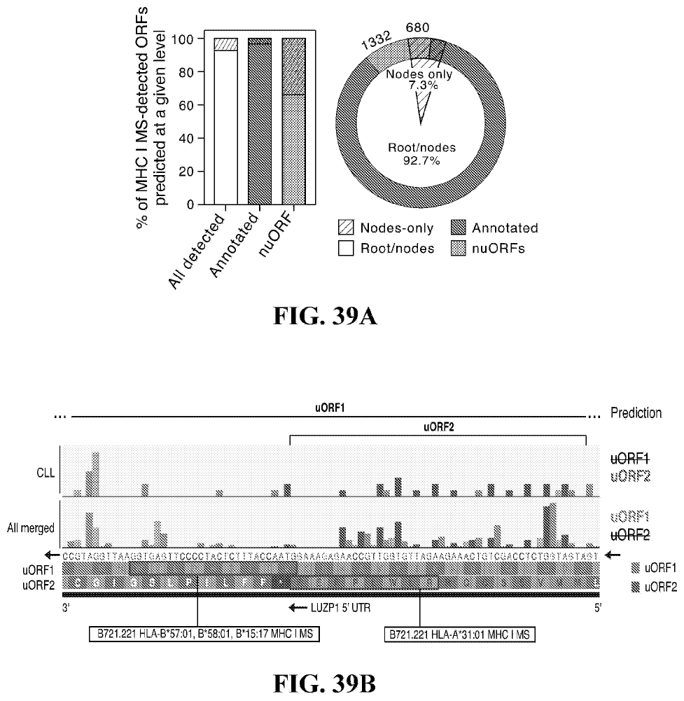

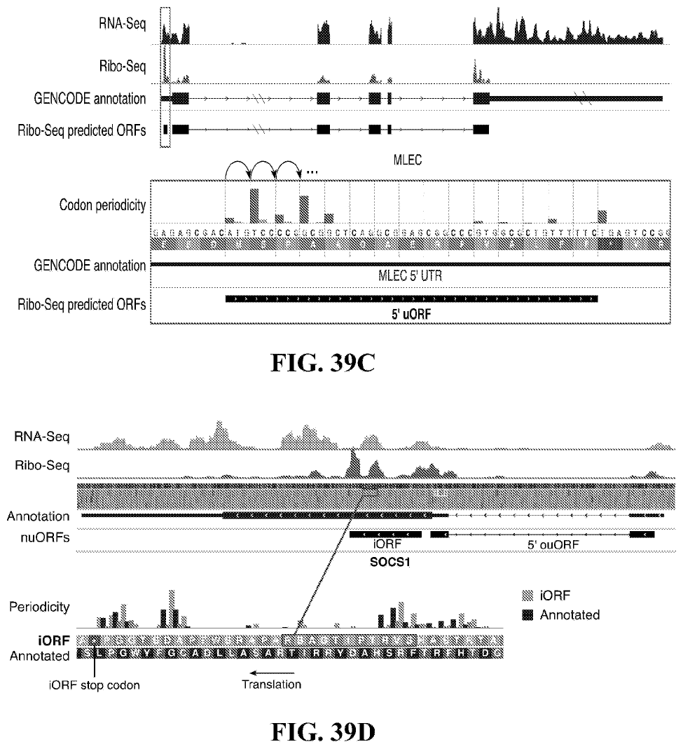

While 97% of MS-detected annotated ORFs could be predicted at the root, 33.8% (680) of the MS-detected nuORFs were exclusively predicted at the nodes in the leaves or branches (FIG. 39A), highlighting the particular value of the hierarchical approach for nuORF identification (FIG. 32C, FIG. 36A). For example, peptides derived from two overlapping 5′uORFs within the 5′UTR of the LUZP1 transcript were detected by MHC IIP MS/MS in B721.221 cells across four different alleles (FIG. 39B). Due to the overlap of these ORFs, one was not predicted at the root, but was predicted in the CLL node, where the other 5′uORF is either translated at much lower levels or not at all.

Many of the MHC I-presented nuORFs (2,093 of 3,261, 64%) overlapped with 5′UTRs and annotated ORFs and thus would have been challenging to identify from RNA-Seq alone, given their short length and proximity to, or overlap with, a longer annotated ORF, but are more readily identifiable by Ribo-seq (FIG. 39B-39D). In fact, peptides from as many as three separate ORFs within one transcript were detected in the MHC I immunopeptidome. For example, in the SOCSJ gene, an important modulator of interferon gamma and JAK-STAT signaling (23), peptides were identified matching to the annotated protein, an internal out-of-frame nuORF (iORF) and a 5′ overlapping uORF (ouORF; FIG. 39C). Thus, nuORFs may be more readily expressed than previously anticipated, and not only generate peptides for MHC I presentation, but may also play other important roles in the cell.

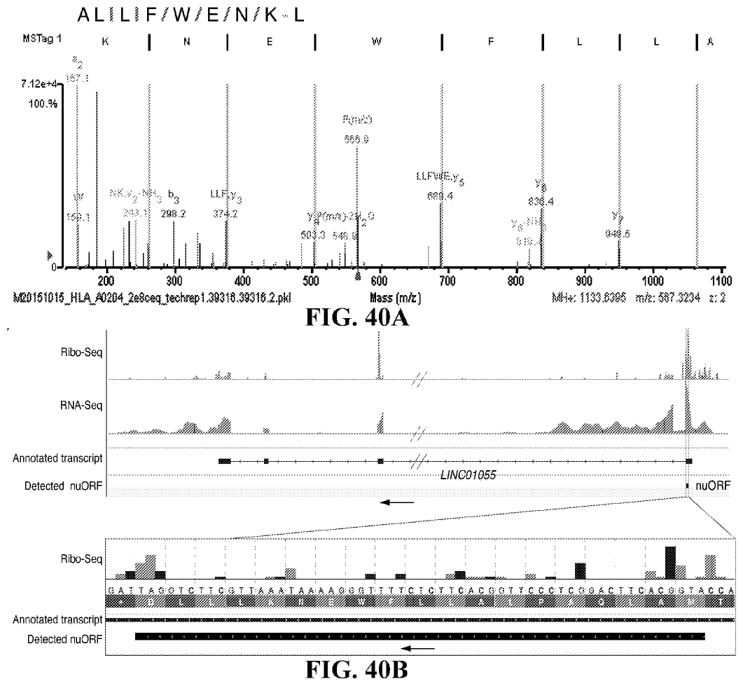

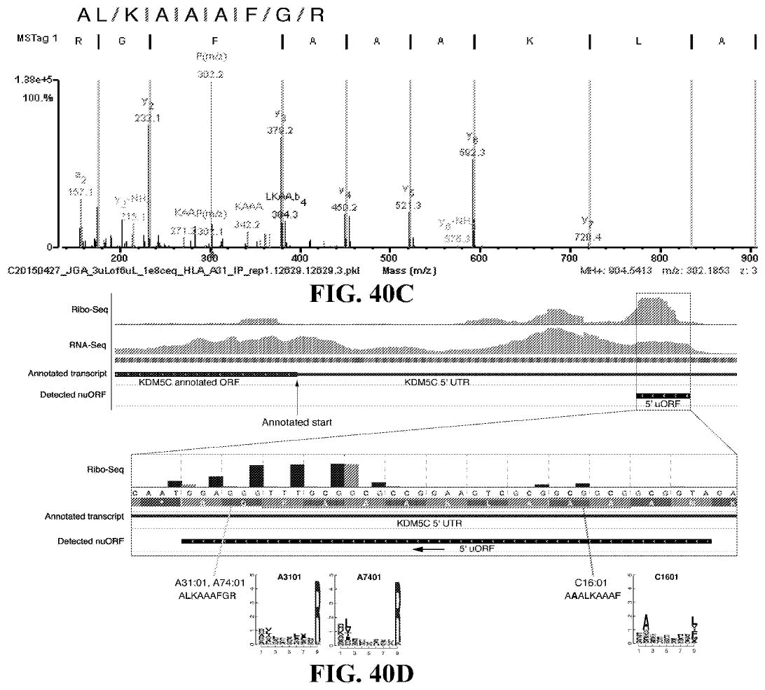

Applicants explored the recent hypothesis that up to 30% of the HLA class I immunopeptidome is not genomically encoded, but is instead derived from proteasomal spliced peptides (24, 25). Based on 9 of our previously published monoallelic HLA class I-expressing datasets (10), 4,426 peptides were proposed to be derived from proteasomal splicing (24). However, in the current data analysis Applicants found 303 nuORF-derived peptides, supported by Ribo-sect, that map to the same MS/SIS spectra as 343 of proposed spliced peptides, in either of two scenarios: (1) for 101 cases, the nuORF-derived peptide sequence is identical to a proposed spliced peptide (FIG. 40A, 40B); or (2) for 202 cases, the partial sequence present in the MS/MS spectrum matched to a nuORF-derived peptide is also consistent with one or more different, yet similar, spliced peptide sequences (FIG. 40C, 40D). Notably, while 84% of nuORF peptides and 94% of annotated peptides had predicted MHC I binding scores over 0.8 (SOM), only 33% of proposed spliced peptides did (FIG. 33G), consistent with reports that many spliced peptides were incorrectly identified (22, 26).

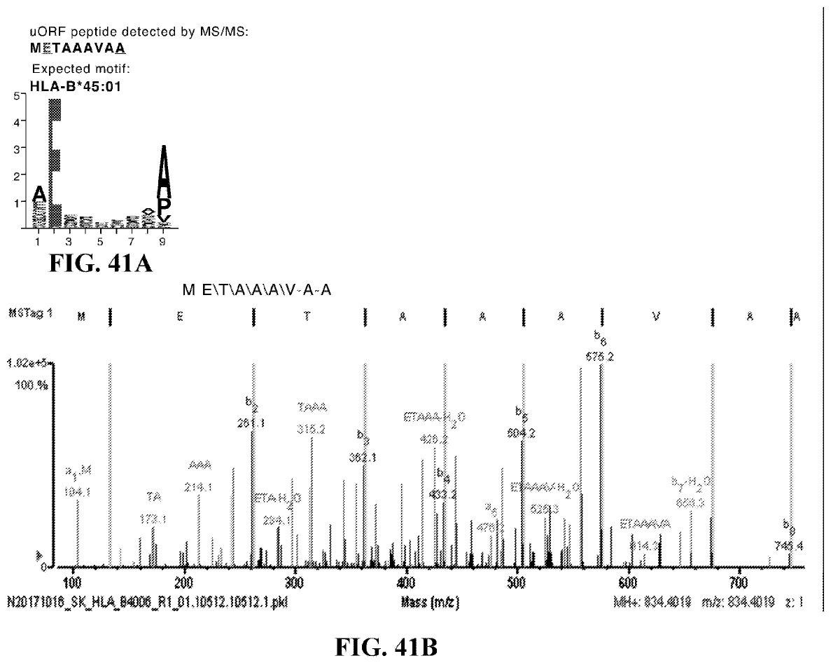

As Applicants previously reported for Ribo-seq predicted nuORFs (17), MHC I MS/MS-detected nuORFs were shorter than annotated ORFs (p<10′ across all nuORF types, t-test) (FIG. 33H). Strikingly, the translated protein products of 26 nuORFs were exactly the same length as their corresponding MHC I-bound antigens, such that they should not require protease processing, as they are ready-made for MHC I presentation. For example, a peptide corresponding to an entire translated 5′ uORF from the 5′ UTR of ARAF is translated at a higher rate than the annotated ARAF protein in B721.221 cells (FIG. 33I), matches the expected motif of HLA-B*45:01, where it was detected (FIG. 41A), and the LC-MS/MS spectra of the peptide closely support the sequence (FIG. 41B). Such short, abundant nuORF proteins may be presented on MHC I more quickly following translation than longer annotated proteins, which require protease processing.

NuORFs were under-represented in whole proteome MS/MS analyses compared to the MHC I immunopeptidome, consistent with previous reports (19, 27). In the whole proteome of B721.221 cells, Applicants identified 205 peptides from 102 nuORFs, representing only 0.1% of all peptides identified and >20-fold less peptides than in the MHC I immunopeptidome (FIG. 33J). For example, Applicants detected only 10 out-of-frame nuORFs and three 5′ uORFs in the whole proteome, compared to 595 and 806 such nuORFs in the MHC I immunopeptidome, respectively (FIG. 33K). Additionally, while 59% of all detected annotated proteins were observed in both the MHC I immunopeptidome and in the whole proteome, only 0.7% of nuORFs were shared (FIG. 33L). Despite comparable levels of translation between nuORFs detected on the MHC I and in the whole proteome (MHC I: 1.23, Whole: 1.42, p=0.26, KS test), the median length of nuORFs detected on the MHC I was far shorter than those detected in the whole proteome (FIG. 33M, 47 vs. 102 amino acids, p<10⁻¹⁶, KS test), suggesting a preference for presentation of shorter nuORFs on MHC I. Thus, the results clearly demonstrate that thousands of nuORFs (Table 1) are translated and contribute an average of 3.3% to the B721.221 MHC I immunopeptidome.

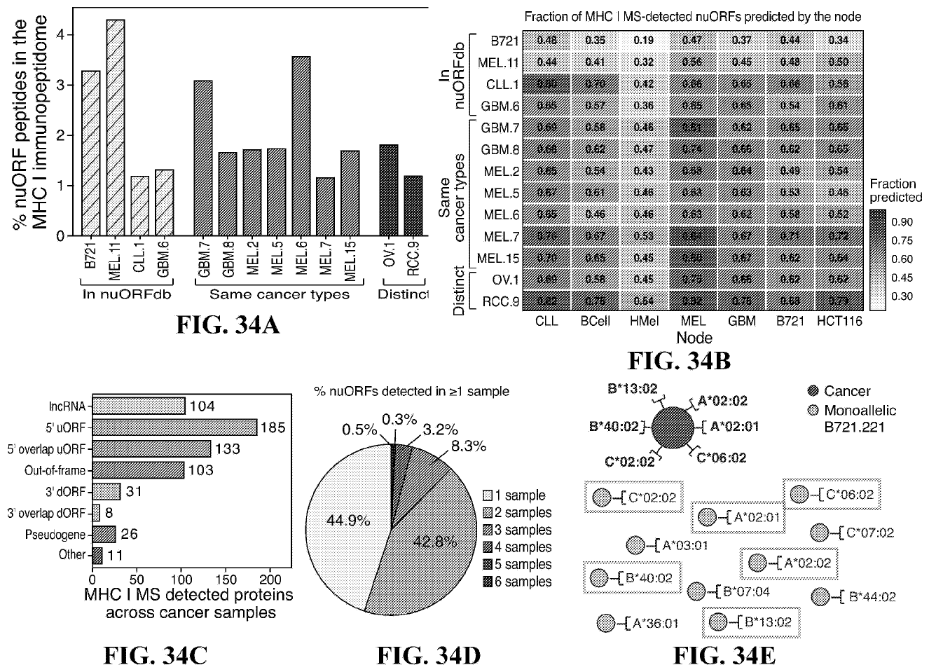

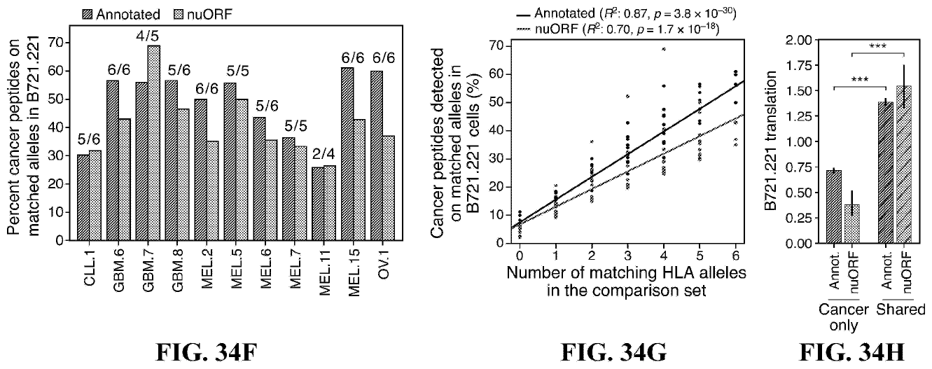

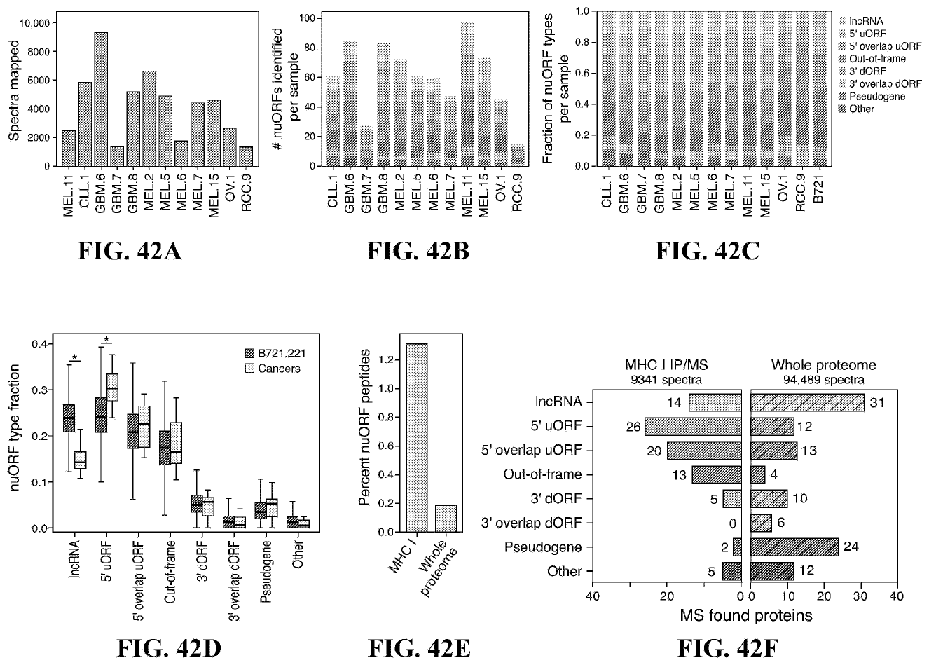

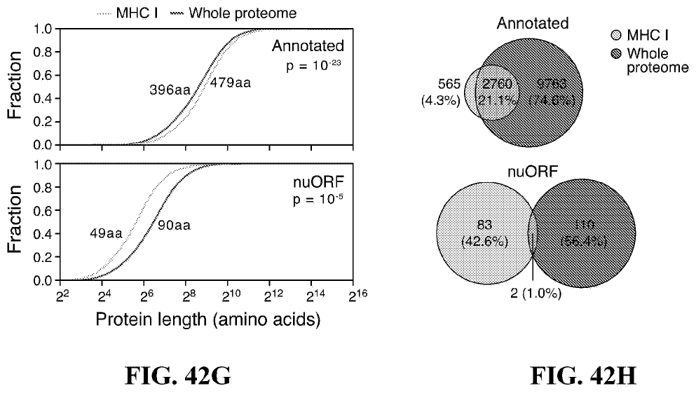

To further investigate nuORFs as a potential source of novel cancer antigens, Applicants used nuORFdb to analyze the MHC I immunopeptidome of 12 cancer samples. On average, ˜1.5-2.3% of the immunopeptidome was assigned to nuORFs, across the melanoma, glioblastoma and CLL samples in nuORFdb (2.3%, n=3), additional melanoma and glioblastoma samples (2.1%, n=7), and renal cell carcinoma and ovarian cancer samples not used to create the database (1.5%, n=2) (FIG. 34A, FIG. 42A). Interestingly, compared to B721.221 cells, lncRNA nuORFs were less frequently observed across these primary human cancer samples (p=10⁻⁶, rank-sum test, SOM), while 5′ uORFs were enriched (p=0.02, rank-sum test, SOM) (FIG. 42B-42D). NuORFs detected across various cancer samples were predicted from multiple nodes used in the generation of nuORFdb, with no single node able to account for all detected nuORFs in a given sample, highlighting the benefits of the present hierarchical ORF prediction approach (FIG. 34B). Importantly, nuORFdb helped detect MHC I presented peptides from translated nuORFs even in samples without any Ribo-seq data, albeit at lower proportion (FIG. 34A). Overall, Applicants detected peptides from 601 unique nuORFs of various types across all cancer immunopeptidomes (FIG. 34C, Table 1). More than half (55%) of the nuORFs were detected in more than one sample, demonstrating that they are not likely derived from random translation, but are translated recurrently across multiple samples (FIG. 34D). As with B721.221 cells, nuORFs were under-represented in the whole proteome of a glioblastoma sample compared to the MHC I immunopeptidome (FIG. 42E, 42F), and those nuORFs detected in the whole proteome were significantly longer than those detected in the MHC I immunopeptidome (FIG. 42G, p=10⁻⁵, KS test), with only 1% overlap between the two sets (FIG. 4211).

TABLE 1

| | | | | B721_ | CLL.1_ | GBM.6_ | GBM.7_ | GBM.8_ | MEL.2_ |
|---|---|---|---|---|---|---|---|---|---|
| ORF_ID | Gene ID | MergeType | PlotType | MHCI | MHCI | MHCI | MHCI | MHCI | MHCI |
| ENST00000 | CTB-158E9.2 | Pseudogene | Pseudogene | — | Detected | — | — | — | — |
| 520639.1_1_ | | | | | | | | | |
| 5:15387334 | | | | | | | | | |
| 8- | | | | | | | | | |
| 153873642: | | | | | | | | | |
| + | | | | | | | | | |
| ENST00000 | CTD-2245F17.3 | Pseudogene | Pseudogene | — | Detected | — | — | — | — |
| 597550.5_1_ | | | | | | | | | |
| 19:5370048 | | | | | | | | | |
| 8- | | | | | | | | | |
| 53703885:+ | | | | | | | | | |
| ENST00000 | DDX18 | ncRNA | lncRNA | — | Detected | — | — | — | — |
| 476149.1_1_ | | Retained | | | | | | | |
| 2:11858822 | | Intron | | | | | | | |
| 9- | | | | | | | | | |
| 118588328: | | | | | | | | | |
| + | | | | | | | | | |
| ENST00000 | PCGF5 | ncRNA | lncRNA | Detected | Detected | — | — | — | — |
| 490164.1_1_ | | Processed | | | | | | | |
| 10:9298250 | | Transcript | | | | | | | |
| 4- | | | | | | | | | |
| 92982606:+ | | | | | | | | | |
| ENST00000 | PCGF5 | Out-of- | Out-of- | — | Detected | — | — | — | — |
| 614189.4_1_ | | Frame | Frame | | | | | | |
| 10:9301111 | | | | | | | | | |
| 1- | | | | | | | | | |
| 93011153:+ | | | | | | | | | |
| ENST00000 | PIM2 | 5′ Overlap | 5′ Overlap | Detected | Detected | — | — | — | — |
| 376509.4_1_ | | uORF | uORF | | | | | | |
| X:48776104- | | | | | | | | | |
| 48776263:- | | | | | | | | | |
| ENST00000 | PPM1K | ncRNA | lnc | Detected | Detected | — | — | — | — |
| 513546.3_1_ | | Retained | RNA | | | | | | |
| 4:89199386- | | Intron | | | | | | | |
| 89199578:- | | | | | | | | | |
| ENST00000 | PRSS16 | 3′ dORF | 3′ dORF | — | Detected | — | — | — | — |
| 421826.6_2_ | | | | | | | | | |
| 6:27223302- | | | | | | | | | |
| 27223911:+ | | | | | | | | | |
| ENST00000 | RP11-54A9.1 | lincRNA | lncRNA | — | Detected | — | — | — | — |
| 553247.1_1_ | | | | | | | | | |
| 12:7668760 | | | | | | | | | |
| 6- | | | | | | | | | |
| 76687717:+ | | | | | | | | | |
| ENST00000 | SEC14L1 | 5′ uORF | 5′ uORF | Detected | Detected | | — | — | — |
| 588880.5_1_ | | | | | | | | | |
| 17:7513710 | | | | | | | | | |
| 5- | | | | | | | | | |
| 75137180:+ | | | | | | | | | |
| ENST00000 | BACH1 | 5′ Overlap | 5′ Overlap | Detected | — | Detected | — | — | — |
| 550131.5_2_ | | uORF | uORF | | | | | | |
| 21:3064277 | | | | | | | | | |
| 5- | | | | | | | | | |
| 30693608:+ | | | | | | | | | |
| ENST00000 | C6orf48 | 5′ uORF | 5′ uORF | Detected | — | Detected | — | — | — |
| 395788.3_1_ | | | | | | | | | |
| 6:31805020- | | | | | | | | | |
| 31805074:+ | | | | | | | | | |
| ENST00000 | CCDC58 | ncRNA | lncRNA | — | — | Detected | — | — | — |
| 466854.5_1_ | | Processed | | | | | | | |
| 3:12208187 | | Transcript | | | | | | | |
| 3- | | | | | | | | | |
| 122084420:- | | | | | | | | | |
| ENST00000 | CCP110 | 5′ uORF | 5′ uORF | Detected | — | Detected | — | — | — |
| 396212.6_1_ | | | | | | | | | |
| 16:1953526 | | | | | | | | | |
| 1- | | | | | | | | | |
| 19535342:+ | | | | | | | | | |
| ENST00000 | CCZ1B | 3′ dORF | 3′ dORF | — | — | Detected | — | — | — |
| 411858.1_1_ | | | | | | | | | |
| 7:6863774- | | | | | | | | | |
| 6864307:- | | | | | | | | | |
| ENST00000 | CGGBP1 | 5′ uORF | 5′ uORF | Detected | — | Detected | — | — | — |
| 309534.10_1 | | | | | | | | | |
| 3:8810729 | | | | | | | | | |
| 1- | | | | | | | | | |
| 88108197:- | | | | | | | | | |
| ENST00000 | CSRP2 | Out-of- | Out-of- | — | — | Detected | — | — | — |
| 547435.1_1_ | | Frame | Frame | | | | | | |
| 12:7725407 | | | | | | | | | |
| 7- | | | | | | | | | |
| 77257079:- | | | | | | | | | |
| ENST00000 | CTBP1 | 5′ uORF | 5′ uORF | Detected | — | Detected | — | — | — |
| 382952.7_2_ | | | | | | | | | |
| 4:1235125- | | | | | | | | | |
| 1235221:- | | | | | | | | | |
| ENST00000 | CTNNB1 | 5′ Overlap | 5′ Overlap | Detected | — | Detected | — | — | — |
| 396185.7_1_ | | uORF | uORF | | | | | | |
| 3:41241009- | | | | | | | | | |
| 41266025:+ | | | | | | | | | |
| ENST00000 | DAXX | 5′ uORF | 5′ uORF | Detected | — | Detected | — | — | — |
| 266000.10_2_ | | | | | | | | | |
| 6:3328971 | | | | | | | | | |
| 1- | | | | | | | | | |
| 33290733:- | | | | | | | | | |
| ENST00000 | DSEL | 5′ uORF | 5′ uORF | — | — | Detected | — | — | — |
| 310045.7_1_ | | | | | | | | | |
| 18:6518203 | | | | | | | | | |
| 2- | | | | | | | | | |
| 65182113:- | | | | | | | | | |
| ENST00000 | EDNRB | 5′ Overlap | 5′ Overlap | — | — | Detected | — | — | — |
| 334286.7_1_ | | uORF | uORF | | | | | | |
| 13:7849271 | | | | | | | | | |
| 5- | | | | | | | | | |
| 78492892:- | | | | | | | | | |
| ENST00000 | EIFIAY | 5′ Overlap | 5′ Overlap | — | — | Detected | — | — | — |
| 382772.3_1_ | | uORF | uORF | | | | | | |
| Y:22737737- | | | | | | | | | |
| 22744490:+ | | | | | | | | | |
| ENST00000 | ERAP2 | Out-of- | Out-of- | Detected | — | Detected | — | — | — |
| 379904.8_1_ | | Frame | Frame | | | | | | |
| 5:96215484- | | | | | | | | | |
| 96215517:+ | | | | | | | | | |
| ENST00000 | ERCC8 | 5′ Overlap | 5′ Overlap | — | — | Detected | — | — | — |
| 381118.7_1_ | | uORF | uORF | | | | | | |
| 5:60224765- | | | | | | | | | |
| 60240875:- | | | | | | | | | |
| ENST00000 | FEM1C | 5′ uORF | 5′ uORF | — | — | Detected | — | — | — |
| 274457.4_1_ | | | | | | | | | |
| 5:11487925 | | | | | | | | | |
| 0- | | | | | | | | | |
| 114879322:- | | | | | | | | | |
| ENST00000 | FGFR1 | 5′ Overlap | 5′ Overlap | — | — | Detected | — | — | — |
| 440174.1_1_ | | uORF | uORF | | | | | | |
| 8:38314980- | | | | | | | | | |
| 38320646:- | | | | | | | | | |
| ENST00000 | GOLGA7 | 3′ dORF | 3′ dORF | — | — | Detected | — | — | — |
| 520817.5_1_ | | | | | | | | | |
| 8:41367338- | | | | | | | | | |
| 4136752 1 :+ | | | | | | | | | |
| ENST00000 | GPAT4 | 5′ uORF | 5′ uORF | Detected | — | Detected | — | — | — |
| 396987.7_1_ | | | | | | | | | |
| 8:41455847- | | | | | | | | | |
| 41455943:+ | | | | | | | | | |
| ENST00000 | GRB10 | 3′ dORF | 3′ dORF | — | — | Detected | — | — | — |
| 398810.6_1_ | | | | | | | | | |
| 7:50660504- | | | | | | | | | |
| 50660642:- | | | | | | | | | |
| ENST00000 | HCN3 | Out-of- | Out-of- | — | — | Detected | — | — | — |
| 467204.1_1_ | | Frame | Frame | | | | | | |
| 1:15525233 | | | | | | | | | |
| 7- | | | | | | | | | |
| 155256975: | | | | | | | | | |
| + | | | | | | | | | |
| ENST00000 | HERPUD2 | 5′ uORF | 5′ uORF | Detected | — | Detected | — | — | — |
| 603731.1_2_ | | | | | | | | | |
| 7:35734066- | | | | | | | | | |
| 35735032:- | | | | | | | | | |
| ENST00000 | LINC01550 | lincRNA | lncRNA | — | — | Detected | — | — | — |
| 499006.6_1_ | | | | | | | | | |
| 14:9843586 | | | | | | | | | |
| 3- | | | | | | | | | |
| 98444319:- | | | | | | | | | |
| ENST00000 | LINC01578 | lincRNA | lncRNA | Detected | — | Detected | — | — | — |
| 557682.6_1_ | | | | | | | | | |
| 15:9342607 | | | | | | | | | |
| 2- | | | | | | | | | |
| 93426198:+ | | | | | | | | | |
| ENST00000 | MKI67 | ncRNA | lncRNA | Detected | — | Detected | — | — | — |
| 478293.1_1_ | | Processed | | | | | | | |
| 10:1299140 | | Transcript | | | | | | | |
| 37- | | | | | | | | | |
| 129924496:- | | | | | | | | | |
| ENST00000 | OCLN | ncRNA | lncRNA | Detected | | Detected | — | — | — |
| 510666.1_1_ | | Retained | | | | | | | |
| 5:68840710- | | Intron | | | | | | | |
| 68849495:+ | | | | | | | | | |
| ENST00000 | OGG1 | 5′ Overlap | 5′ Overlap | — | — | Detected | — | — | — |
| 302036.11_2 | | uORF | uORF | | | | | | |
| 3:9791692- | | | | | | | | | |
| 9792019:+ | | | | | | | | | |
| ENST00000 | PARD3 | 5′ uORF | 5′ uORF | Detected | — | Detected | — | — | — |
| 545260.5_2_ | | | | | | | | | |
| 10:3510404 | | | | | | | | | |
| 2- | | | | | | | | | |
| 35104177:- | | | | | | | | | |
| ENST00000 | PATL1 | Out-of- | Out-of- | Detected | — | Detected | — | — | — |
| 300146.9_1_ | | Frame | Frame | | | | | | |
| 11:5942678 | | | | | | | | | |
| 6- | | | | | | | | | |
| 59434427:- | | | | | | | | | |
| ENST00000 | PCDHGC3 | Out-of- | Out-of- | — | — | Detected | — | — | — |
| 611950.1_1_ | | Frame | Frame | | | | | | |
| 5:14085578 | | | | | | | | | |
| 6- | | | | | | | | | |
| 140855918: | | | | | | | | | |
| + | | | | | | | | | |
| ENST00000 | PSMD2 | ncRNA | lncRNA | Detected | — | Detected | — | — | — |
| 487475.5_1_ | | Retained | | | | | | | |
| 3:18401707 | | Intron | | | | | | | |
| 6- | | | | | | | | | |
| 184017693: | | | | | | | | | |
| + | | | | | | | | | |
| ENST00000 | PTGFRN | 5′ Overlap | 5′ Overlap | — | — | Detected | — | — | — |
| 393203.2_1_ | | uORF | uORF | | | | | | |
| 1:11745273 | | | | | | | | | |
| 9- | | | | | | | | | |
| 117484357: | | | | | | | | | |
| + | | | | | | | | | |
| ENST00000 | PTPRZ1 | Out-of | Out-of | — | — | Detected | — | — | — |
| 449182.1_1_ | | Frame | Frame | | | | | | |
| 7:12161261 | | | | | | | | | |
| 2- | | | | | | | | | |
| 121612669: | | | | | | | | | |
| + | | | | | | | | | |
| ENST00000 | RFX5 | 5′ Overlap | 5′ Overlap | Detected | — | Detected | — | — | — |
| 450506.5_1_ | | uORF | uORF | | | | | | |
| 1:15131841 | | | | | | | | | |
| 4- | | | | | | | | | |
| 151318804:- | | | | | | | | | |
| ENST00000 | RPL19 | 5′ Overlap | 5′ Overlap | Detected | — | Detected | — | — | — |
| 225430.8_2_ | | uORF | uORF | | | | | | |
| 17:3735654 | | | | | | | | | |
| 2- | | | | | | | | | |
| 37357495:+ | | | | | | | | | |
| ENST00000 | RPL23AP7 | Pseudogene | Pseudogene | — | — | Detected | — | — | — |
| 416673.6_1_ | | | | | | | | | |
| 2:11436976 | | | | | | | | | |
| 3- | | | | | | | | | |
| 114384446:- | | | | | | | | | |
| ENST00000 | SLC25A40 | 5′ uORF | 5′ uORF | Detected | — | Detected | — | — | — |
| 444363.5_1_ | | | | | | | | | |
| 7:87488057- | | | | | | | | | |
| 87505510:- | | | | | | | | | |
| ENST00000 | SNHG5 | lincRNA | lncRNA | — | — | Detected | — | — | — |
| 587692.5_1_ | | | | | | | | | |
| 6:86388339- | | | | | | | | | |
| 86388411:- | | | | | | | | | |
| ENST00000 | SPAG9 | 5′ uORF | 5′ uORF | — | — | Detected | — | — | — |
| 510283.5_1_ | | | | | | | | | |
| 17:4912406 | | | | | | | | | |
| 5- | | | | | | | | | |
| 49124107:- | | | | | | | | | |
| ENST00000 | SSCA1-AS1 | TEC | Other | — | — | Detected | — | — | — |
| 623234.1_1_ | | | | | | | | | |
| 11:6533735 | | | | | | | | | |
| 0- | | | | | | | | | |
| 65337503:- | | | | | | | | | |
| ENST00000 | TRIO | 5′ Overlap | 5′ Overlap | — | — | Detected | — | — | — |
| 515710.1_1_ | | uORF | uORF | | | | | | |
| 5:14441357- | | | | | | | | | |
| 14465986:+ | | | | | | | | | |
| ENST00000 | TVP23B | ncRNA | lncRNA | — | — | Detected | — | — | — |
| 482741.1_1_ | | Retained | | | | | | | |
| 17:1870094 | | Intron | | | | | | | |
| 0- | | | | | | | | | |
| 18702212:+ | | | | | | | | | |
| ENST00000 | UFC1 | ncRNA | lncRNA | Detected | — | Detected | — | — | — |
| 483191.5_1_ | | Processed | | | | | | | |
| 1:16112378 | | Transcript | | | | | | | |
| 4- | | | | | | | | | |
| 161128238: | | | | | | | | | |
| + | | | | | | | | | |
| ENST00000 | USP39 | Out-of- | Out-of- | Detected | — | Detected | — | — | — |
| 409470.5_2_ | | Frame | Frame | | | | | | |
| 2:85843553- | | | | | | | | | |
| 85850780:+ | | | | | | | | | |
| ENST00000 | ZCCHC11 | 5′ uORF | 5′ uORF | Detected | — | Detected | — | — | — |
| 371544.7_1_ | | | | | | | | | |
| 1:53018618- | | | | | | | | | |
| 53018768:- | | | | | | | | | |
| ENST00000 | ZFP69B | 5′ uORF | 5′ uORF | — | — | Detected | — | — | — |
| 469416.1_1_ | | | | | | | | | |
| 1:40916330- | | | | | | | | | |
| 40916492:+ | | | | | | | | | |
| ENST00000 | ZNF146 | Out-of- | Out-of- | Detected | — | Detected | — | — | — |
| 443387.2_1_ | | Frame | Frame | | | | | | |
| 19:3672715 | | | | | | | | | |
| 1- | | | | | | | | | |
| 36727859:+ | | | | | | | | | |
| ENST00000 | ZNF521 | 5′ Overlap | 5′ Overlap | — | — | Detected | — | — | — |
| 361524.7_1_ | | uORF | uORF | | | | | | |
| 18:2290214 | | | | | | | | | |
| 1- | | | | | | | | | |
| 22932115:- | | | | | | | | | |
| ENST00000 | ZNF614 | Nonsense | Other | — | — | Detected | — | — | — |
| 595189.5_1_ | | Mediated | | | | | | | |
| 19:5252094 | | Decay | | | | | | | |
| 5- | | | | | | | | | |
| 52529004:- | | | | | | | | | |
| T028857_1: | nan | lincRNA | lncRNA | Detected | — | Detected | — | — | — |
| 205417257- | | | | | | | | | |
| 205417461: | | | | | | | | | |
| + | | | | | | | | | |
| T302015_6: | nan | TUCP | Other | — | — | Detected | — | — | — |
| 33650185- | | | | | | | | | |
| 33650641:- | | | | | | | | | |
| ENST00000 | DDX3X | Out-of- | Out-of- | Detected | Detected | Detected | — | — | — |
| 399959.6_1_ | | Frame | Frame | | | | | | |
| X:41200742- | | | | | | | | | |
| 41202586:+ | | | | | | | | | |
| ENST00000 | MORFF4L1 | Out-of- | Out-of- | Detected | Detected | Detected | — | — | — |
| 558746.5_1_ | | Frame | Frame | | | | | | |
| 15:7916539 | | | | | | | | | |
| 3- | | | | | | | | | |
| 79172860:+ | | | | | | | | | |
| ENST00000 | ATF4 | 5′ Overlap | 5′ Overlap | — | — | — | Detected | — | — |
| 404241.6_1_ | | uORF | uORF | | | | | | |
| 22:3991675 | | | | | | | | | |
| 1- | | | | | | | | | |
| 39917530:+ | | | | | | | | | |
| ENST00000 | CENPBD1P1 | Pseudogene | Pseudogene | Detected | — | — | Detected | — | — |
| 493504.1_1_ | | | | | | | | | |
| 19:5908694 | | | | | | | | | |
| 2- | | | | | | | | | |
| 59092775:+ | | | | | | | | | |
| ENST00000 | CKAP2 | Out-of- | Out-of- | Detected | — | — | Detected | — | — |
| 378037.9_1_ | | Frame | Frame | | | | | | |
| 13:5303070 | | | | | | | | | |
| 8- | | | | | | | | | |
| 53035053:+ | | | | | | | | | |
| ENST00000 | CORO1C | 5′ Overlap | 5′ Overlap | Detected | — | — | Detected | — | — |
| 261401.7_1_ | | uORF | uORF | | | | | | |
| 12:1090950 | | | | | | | | | |
| 90- | | | | | | | | | |
| 109125303:- | | | | | | | | | |
| ENST00000 | GMCL1 | 5′ uORF | 5′ uORF | Detected | — | — | Detected | — | — |
| 282570.3_1_ | | | | | | | | | |
| 2:70056849- | | | | | | | | | |
| 70056930:+ | | | | | | | | | |
| ENST00000 | HIST1H1C | Out-of- | Out-of- | Detected | — | — | Detected | — | — |
| 343677.3_1_ | | Frame | Frame | | | | | | |
| 6:26056391- | | | | | | | | | |
| 26056457:- | | | | | | | | | |
| ENST00000 | JTB | 5′ uORF | 5′ uORF | Detected | — | — | Detected | — | — |
| 271843.8_1_ | | | | | | | | | |
| 1:15395000 | | | | | | | | | |
| 7- | | | | | | | | | |
| 153950046:- | | | | | | | | | |
| ENST00000 | KLHDC8A | 5′ uORF | 5′ uORF | — | — | — | Detected | — | — |
| 539253.5_1_ | | | | | | | | | |
| 1:20531279 | | | | | | | | | |
| 2- | | | | | | | | | |
| 205312870:- | | | | | | | | | |
| ENST00000 | LINC00639 | lincRNA | lncRNA | — | — | — | Detected | — | — |
| 553932.5_1_ | | | | | | | | | |
| 14:3930725 | | | | | | | | | |
| 6- | | | | | | | | | |
| 39307550:- | | | | | | | | | |
| ENST00000 | MGAT1 | ncRNA | lncRNA | — | — | — | Detected | — | — |
| 508090.1_1_ | | Processed | | | | | | | |
| 5:18023591 | | Transcript | | | | | | | |
| 6- | | | | | | | | | |
| 180242424:- | | | | | | | | | |
| ENST00000 | MORF4L2 | 5′ uORF | 5′ uORF | Detected | — | — | Detected | — | — |
| 422154.6_1_ | | | | | | | | | |
| X:10294015 | | | | | | | | | |
| 8- | | | | | | | | | |
| 102941669:- | | | | | | | | | |
| ENST00000 | PCDHGC3 | 5′ uORF | 5′ uORF | — | — | — | Detected | — | — |
| 611950.1_1_ | | | | | | | | | |
| 5:14085555 | | | | | | | | | |
| 6- | | | | | | | | | |
| 140855619: | | | | | | | | | |
| + | | | | | | | | | |
| ENST00000 | RPL7AP34 | Pseudogene | Pseudogene | Detected | — | — | Detected | — | — |
| 416120.1_1_ | | | | | | | | | |
| 6:64259201- | | | | | | | | | |
| 64259360:- | | | | | | | | | |
| ENST00000 | SRCAP | 5′ Overlap | 5′ Overlap | Detected | — | — | Detected | — | — |
| 411466.6_3_ | | uORF | uORF | | | | | | |
| 16:3070955 | | | | | | | | | |
| 3- | | | | | | | | | |
| 30715400:+ | | | | | | | | | |
| ENST00000 | SSBP4 | 5′ uORF | 5′ uORF | Detected | — | — | Detected | — | — |
| 270061.11_1_ | | | | | | | | | |
| 19:185303 | | | | | | | | | |
| 29- | | | | | | | | | |
| 18530365:+ | | | | | | | | | |
| ENST00000 | WBP1 | 5′ uORF | 5′ uORF | Detected | — | — | Detected | — | — |
| 233615.6_2_ | | | | | | | | | |
| 2:74685568- | | | | | | | | | |
| 74685718:+ | | | | | | | | | |
| ENST00000 | ZC3H11A | 5′ uORF | 5′ uORF | — | — | — | Detected | — | — |
| 367210.2_2_ | | | | | | | | | |
| 1:20377143 | | | | | | | | | |
| 5- | | | | | | | | | |
| 203771690: | | | | | | | | | |
| + | | | | | | | | | |
| ENST00000 | ZNF32 | 5′ Overlap | 5′ Overlap | — | — | — | Detected | — | — |
| 485351.1_1_ | | uORF | uORF | | | | | | |
| 10:4414151 | | | | | | | | | |
| 0- | | | | | | | | | |
| 44141681:- | | | | | | | | | |
| TCONS_00 | other | lincRNA | lncRNA | Detected | — | — | Detected | — | — |
| 030037_GL | | | | | | | | | |
| 000220.1:97 | | | | | | | | | |
| 269-97317:+ | | | | | | | | | |
| ENST00000 | CCSER1 | 5′ uORF | 5′ uORF | — | — | Detected | Detected | — | — |
| 505073.5_1_ | | | | | | | | | |
| 4:91048743- | | | | | | | | | |
| 91048815:+ | | | | | | | | | |
| ENST00000 | ABHD17A | 5′ uORF | 5′ uORF | Detected | — | — | — | Detected | — |
| 292577.11_1_ | | | | | | | | | |
| 19:188171 | | | | | | | | | |
| 2-1885478:- | | | | | | | | | |
| ENST00000 | AC105760.2 | Antisense | lncRNA | — | — | — | — | Detected | — |
| 418430.1_1_ | | | | | | | | | |
| 2:23799319 | | | | | | | | | |
| 4- | | | | | | | | | |
| 237993419:- | | | | | | | | | |
| ENST00000 | AGGF1 | 5′ Overlap | 5′ Overlap | — | — | — | — | Detected | — |
| 312916.11_1_ | | uORF | uORF | | | | | | |
| 5:7632632 | | | | | | | | | |
| 3- | | | | | | | | | |
| 76326545:+ | | | | | | | | | |
| ENST00000 | ANKRD34A | 5′ uORF | 5′ uORF | — | — | — | — | Detected | — |
| 606888.2_1_ | | | | | | | | | |
| 1:14547257 | | | | | | | | | |
| 5- | | | | | | | | | |
| 145473224: | | | | | | | | | |
| + | | | | | | | | | |
| ENST00000 | ATF2 | 5′ uORF | 5′ uORF | Detected | — | — | — | Detected | — |
| 264110.6_1_ | | | | | | | | | |
| 2:17600118 | | | | | | | | | |
| 9- | | | | | | | | | |
| 176015807:- | | | | | | | | | |
| ENST00000 | CCDC80 | 5′ Overlap | 5′ Overlap | — | — | — | — | Detected | — |
| 475181.1_1_ | | uORF | uORF | | | | | | |
| 3:11235866 | | | | | | | | | |
| 6- | | | | | | | | | |
| 112359550:- | | | | | | | | | |
| ENST00000 | CHRNB1 | 5′ uORF | 5′ uORF | Detected | — | — | — | Detected | — |
| 575379.1_1_ | | | | | | | | | |
| 17:7358950- | | | | | | | | | |
| 7359124:+ | | | | | | | | | |
| ENST00000 | CNIH3 | 5′ uORF | 5′ uORF | — | — | — | — | Detected | — |
| 272133.3_1_ | | | | | | | | | |
| 1:22480409 | | | | | | | | | |
| 8- | | | | | | | | | |
| 224804761: | | | | | | | | | |
| + | | | | | | | | | |
| ENST00000 | CTDSP2 | 3′ dORF | 3′ dORF | Detected | — | — | — | Detected | — |
| 398073.6_1_ | | | | | | | | | |
| 12:5821689 | | | | | | | | | |
| 1- | | | | | | | | | |
| 58217005:- | | | | | | | | | |
| ENST00000 | EED | 5′ Overlap | 5′ Overlap | Detected | — | — | — | Detected | — |
| 263360.10_1_ | | uORF | uORF | | | | | | |
| 11:859559 | | | | | | | | | |
| 88- | | | | | | | | | |
| 85956345:+ | | | | | | | | | |
| ENST00000 | EIF1B | 5′ Overlap | 5′ Overlap | — | — | — | — | Detected | — |
| 232905.3_1_ | | uORF | uORF | | | | | | |
| 3:40351265- | | | | | | | | | |
| 40352449:+ | | | | | | | | | |
| ENST00000 | EIF1P7 | Pseudogene | Pseudogene | — | — | — | — | Detected | — |
| 487533.1_1_ | | | | | | | | | |
| 2:9411660- | | | | | | | | | |
| 9411717:- | | | | | | | | | |
| ENST00000 | FAM118B | Out-of- | Out-of- | Detected | — | — | — | Detected | — |
| 360194.8_2_ | | Frame | Frame | | | | | | |
| 11:1261108 | | | | | | | | | |
| 47- | | | | | | | | | |
| 126120518: | | | | | | | | | |
| + | | | | | | | | | |
| ENST00000 | GAPVD1 | 5′ uORF | 5′ uORF | — | — | — | — | Detected | — |
| 497580.5_1_ | | | | | | | | | |
| 9:12802412 | | | | | | | | | |
| 4- | | | | | | | | | |
| 128024166: | | | | | | | | | |
| + | | | | | | | | | |
| ENST00000 | GLUL | 5′ uORF | 5′ uORF | — | — | — | — | Detected | — |
| 339526.8_1_ | | | | | | | | | |
| 1:18235986 | | | | | | | | | |
| 6- | | | | | | | | | |
| 182360031:- | | | | | | | | | |
| ENST00000 | HERPUD2 | 5′ uORF | 5′ uORF | — | — | — | — | Detected | — |
| 603731.1_2_ | | | | | | | | | |
| 7:35734046- | | | | | | | | | |
| 35735162:- | | | | | | | | | |
| ENST00000 | IGFBP3 | 5′ Overlap | 5′ Overlap | — | — | — | — | Detected | — |
| 381086.9_1_ | | uORF | uORF | | | | | | |
| 7:45960389- | | | | | | | | | |
| 45960869:- | | | | | | | | | |
| ENST00000 | JMJD8 | ncRNA | lncRNA | — | — | — | — | Detected | — |
| 568689.5_1_ | | Retained | | | | | | | |
| 16:733528- | | Intron | | | | | | | |
| 734236:- | | | | | | | | | |
| ENST00000 | JOSD2 | 5′ Overlap | 5′ Overlap | — | — | — | — | Detected | — |
| 601423.5_2_ | | uORF | uORF | | | | | | |
| 19:5101093 | | | | | | | | | |
| 6- | | | | | | | | | |
| 51014415:- | | | | | | | | | |
| ENST00000 | KEAP1 | 5′ Overlap | 5′ Overlap | — | — | — | — | Detected | — |
| 592055.2_2_ | | uORF | uORF | | | | | | |
| 19:1061068 | | | | | | | | | |
| 9- | | | | | | | | | |
| 10613773:- | | | | | | | | | |
| ENST00000 | KRT10 | 3′ dORF | 3′ dORF | Detected | — | — | — | Detected | — |
| 635956.1_1_ | | | | | | | | | |
| 17:3897438 | | | | | | | | | |
| 0- | | | | | | | | | |
| 38974494:- | | | | | | | | | |
| ENST00000 | LINC00461 | lincRNA | lncRNA | Detected | — | — | — | Detected | — |
| 505030.5_1_ | | | | | | | | | |
| 5:87969013- | | | | | | | | | |
| 87969124:- | | | | | | | | | |
| ENST00000 | MCC | 5′ uORF | 5′ uORF | — | — | — | — | Detected | — |
| 302475.8_1_ | | | | | | | | | |
| 5:11263015 | | | | | | | | | |
| 0- | | | | | | | | | |
| 112630183:- | | | | | | | | | |
| ENST00000 | MESDC1 | 5′ uORF | 5′ uORF | Detected | — | — | — | Detected | — |
| 267984.3_1_ | | | | | | | | | |
| 15:8129357 | | | | | | | | | |
| 1- | | | | | | | | | |
| 81293748:+ | | | | | | | | | |
| ENST00000 | MORF4L2 | 5′ uORF | 5′ uORF | Detected | — | — | — | Detected | — |
| 418819.5_3_ | | | | | | | | | |
| X:10293350 | | | | | | | | | |
| 1- | | | | | | | | | |
| 102939656:- | | | | | | | | | |
| ENST00000 | NEK1 | 5′ uORF | 5′ uORF | Detected | — | — | — | Detected | — |
| 439128.6_2_ | | | | | | | | | |
| 4:17053319 | | | | | | | | | |
| 0- | | | | | | | | | |
| 170533223:- | | | | | | | | | |
| ENST00000 | NRBF2 | 5′ Overlap | 5′ Overlap | — | — | — | — | Detected | — |
| 277746.10_1_ | | uORF | uORF | | | | | | |
| 10:648930 | | | | | | | | | |
| 73- | | | | | | | | | |
| 64911919:+ | | | | | | | | | |
| ENST00000 | NSRP1 | ncRNA | lncRNA | Detected | — | — | — | Detected | — |
| 540900.7_1_ | | Processed | | | | | | | |
| 17:2844385 | | Transcript | | | | | | | |
| 8- | | | | | | | | | |
| 28499569:+ | | | | | | | | | |
| ENST00000 | POPDC3 | ncRNA | lncRNA | — | — | — | — | Detected | — |
| 474760.1_1_ | | Processed | | | | | | | |
| 6:10560992 | | Transcript | | | | | | | |
| 8- | | | | | | | | | |
| 105627738:- | | | | | | | | | |
| ENST00000 | PRR13 | 5′ Overlap | 5′ Overlap | — | — | — | — | Detected | — |
| 549924.5_1_ | | uORF | uORF | | | | | | |
| 12:5383557 | | | | | | | | | |
| 6- | | | | | | | | | |
| 53837250:+ | | | | | | | | | |
| ENST00000 | RAB10 | 5′ Overlap | 5′ Overlap | Detected | — | — | — | Detected | — |
| 495146.5_1_ | | uORF | uORF | | | | | | |
| 2:26257410- | | | | | | | | | |
| 26257569:+ | | | | | | | | | |
| ENST00000 | RP11-231C14.7 | lincRNA | lncRNA | Detected | — | — | — | Detected | — |
| 604430.1_1_ | | | | | | | | | |
| 16:2933228 | | | | | | | | | |
| 5- | | | | | | | | | |
| 29370594:+ | | | | | | | | | |
| ENST00000 | RP11-712L6.5 | Antisense | lncRNA | Detected | — | — | — | Detected | — |
| 524964.1_1_ | | | | | | | | | |
| 11:1261643 | | | | | | | | | |
| 66- | | | | | | | | | |
| 126174190:- | | | | | | | | | |
| ENST00000 | RP13-1032I1.7 | Antisense | lncRNA | Detected | — | — | — | Detected | — |
| 575312.1_1_ | | | | | | | | | |
| 17:7967002 | | | | | | | | | |
| 8- | | | | | | | | | |
| 79670313:- | | | | | | | | | |
| ENST00000 | SEMA6A | Out-of- | Out-of- | — | — | — | — | Detected | — |
| 343348.10_1_ | | Frame | Frame | | | | | | |
| 5:1158379 | | | | | | | | | |
| 42- | | | | | | | | | |
| 115840549:- | | | | | | | | | |
| ENST00000 | SEPT11 | ncRNA | lncRNA | — | — | — | — | Detected | — |
| 512333.1_1_ | | Processed | | | | | | | |
| 4:77909015- | | Transcript | | | | | | | |
| 77926783:+ | | | | | | | | | |
| ENST00000 | SIK2 | 5′ Overlap | 5′ Overlap | — | — | — | — | Detected | — |
| 304987.3_1_ | | uORF | uORF | | | | | | |
| 11:1114732 | | | | | | | | | |
| 65- | | | | | | | | | |
| 111486986: | | | | | | | | | |
| + | | | | | | | | | |
| ENST00000 | SIPA1L2 | 5′ uORF | 5′ uORF | — | — | — | — | Detected | — |
| 366630.5_1_ | | | | | | | | | |
| 1:23265123 | | | | | | | | | |
| 7- | | | | | | | | | |
| 232651315:- | | | | | | | | | |
| ENST00000 | SLC25A5 | ncRNA | lncRNA | Detected | — | — | — | Detected | — |
| 460013.1_1_ | | Processed | | | | | | | |
| X:11860332 | | Transcript | | | | | | | |
| 3- | | | | | | | | | |
| 118603678: | | | | | | | | | |
| + | | | | | | | | | |
| ENST00000 | SMG5 | Out-of- | Out-of- | Detected | — | — | — | Detected | — |
| 361813.5_1_ | | Frame | Frame | | | | | | |
| 1:15624687 | | | | | | | | | |
| 8- | | | | | | | | | |
| 156247780:- | | | | | | | | | |
| ENST00000 | SOX2-OT | Sense | Other | — | — | — | — | Detected | — |
| 492337.5_1_ | | Overlapping | | | | | | | |
| 3:18145800 | | | | | | | | | |
| 7- | | | | | | | | | |
| 181458058: | | | | | | | | | |
| + | | | | | | | | | |
| ENST00000 | SPRY1 | Out-of- | Out-of- | — | — | — | — | Detected | — |
| 610581.4_1_ | | Frame | Frame | | | | | | |
| 4:12432304 | | | | | | | | | |
| 1- | | | | | | | | | |
| 124323077: | | | | | | | | | |
| + | | | | | | | | | |
| ENST00000 | STAMBP | 5′ Overlap | 5′ Overlap | Detected | — | — | — | Detected | — |
| 394070.6_1_ | | uORF | uORF | | | | | | |
| 2:74056525- | | | | | | | | | |
| 74057988:+ | | | | | | | | | |
| ENST00000 | TP53 | 5′ Overlap | 5′ Overlap | — | — | — | — | Detected | — |
| 635293.1_1_ | | uORF | uORF | | | | | | |
| 17:7579872- | | | | | | | | | |
| 7590731:- | | | | | | | | | |
| ENST00000 | TTC9C | 5′ uORF | 5′ uORF | Detected | — | — | — | Detected | — |
| 294161.10_1_ | | | | | | | | | |
| 11:624960 | | | | | | | | | |
| 07- | | | | | | | | | |
| 62496118:+ | | | | | | | | | |
| ENST00000 | TTI2 | 5′ uORF | 5′ uORF | Detected | — | — | — | Detected | — |
| 360742.9_1_ | | | | | | | | | |
| 8:33370539- | | | | | | | | | |
| 33370572:- | | | | | | | | | |
| ENST00000 | UQCR11 | 5′ Overlap | 5′ Overlap | Detected | — | — | — | Detected | — |
| 585671.2_1_ | | uORF | uORF | | | | | | |
| 19:1599451- | | | | | | | | | |
| 1605445:- | | | | | | | | | |
| ENST00000 | USP39 | 3′Overlap | 5′ Overlap | — | — | — | — | Detected | — |
| 455732.5_1_ | | dORF | dORF | | | | | | |
| 2:85843288- | | | | | | | | | |
| 85850811:+ | | | | | | | | | |
| ENST00000 | VIT | 5′ uORF | 5′ uORF | — | — | — | — | Detected | — |
| 457137.6_1_ | | | | | | | | | |
| 2:36924015- | | | | | | | | | |
| 36924090:+ | | | | | | | | | |
| ENST00000 | WWP1 | 5′ uORF | 5′ uORF | — | — | — | — | Detected | — |
| 517970.5_1_ | | | | | | | | | |
| 8:87381186- | | | | | | | | | |
| 87381258:+ | | | | | | | | | |
| ENST00000 | YPEL3 | ncRNA | lncRNA | Detected | — | — | — | Detected | — |
| 565479.5_1_ | | Processed | | | | | | | |
| 16:3010624 | | Transcript | | | | | | | |
| 9- | | | | | | | | | |
| 30106646:- | | | | | | | | | |
| ENST00000 | ZFAND5 | ncRNA | lncRNA | — | — | — | — | Detected | — |
| 488164.5_1_ | | Processed | | | | | | | |
| 9:74975552- | | Transcript | | | | | | | |
| 74978393:- | | | | | | | | | |
| ENST00000 | ZFP69B | 5′ uORF | 5′ uORF | — | — | — | — | Detected | — |
| 484445.5_1_ | | | | | | | | | |
| 1:40915787- | | | | | | | | | |
| 40916492:+ | | | | | | | | | |
| ENST00000 | ZFYVE1 | 5′ Overlap | 5′ Overlap | — | — | — | — | Detected | — |
| 553891.5_1_ | | uORF | uORF | | | | | | |
| 14:7349113 | | | | | | | | | |
| 6- | | | | | | | | | |
| 73491307:- | | | | | | | | | |
| ENST00000 | ZMYND8 | 5′ Overlap | 5′ Overlap | — | — | — | — | Detected | — |
| 355972.8_1_ | | uORF | uORF | | | | | | |
| 20:4597665 | | | | | | | | | |
| 3- | | | | | | | | | |
| 45985423:- | | | | | | | | | |
| ENST00000 | ZNF568 | 5′ uORF | 5′ uORF | Detected | — | — | — | Detected | — |
| 427117.5_1_ | | | | | | | | | |
| 19:3740732 | | | | | | | | | |
| 3- | | | | | | | | | |
| 37407440:+ | | | | | | | | | |
| T056801_11 | nan | TUCP | Other | Detected | — | — | — | Detected | — |
| :32913968- | | | | | | | | | |
| 32914553:- | | | | | | | | | |
| T184311_2: | nan | lincRNA | lncRNA | — | — | — | — | Detected | — |
| 9983205- | | | | | | | | | |
| 9983367:- | | | | | | | | | |
| ENST00000 | CCDC34 | Out-of- | Out-of- | Detected | — | Detected | — | Detected | — |
| 328697.10_2_ | | Frame | Frame | | | | | | |
| 11:273844 | | | | | | | | | |
| 10- | | | | | | | | | |
| 27384560:- | | | | | | | | | |
| ENST00000 | CD276 | 5′ Overlap | 5′ Overlap | — | — | Detected | — | Detected | — |
| 318443.9_1_ | | uORF | uORF | | | | | | |
| 15:7397677 | | | | | | | | | |
| 5- | | | | | | | | | |
| 73994724:+ | | | | | | | | | |
| ENST00000 | CD93 | 5′ Overlap | 5′ Overlap | Detected | — | Detected | — | Detected | — |
| 246006.4_1_ | | uORF | uORF | | | | | | |
| 20:2306675 | | | | | | | | | |
| 8- | | | | | | | | | |
| 23066941:- | | | | | | | | | |
| ENST00000 | CTCF | 5′ Overlap | 5′ Overlap | Detected | — | Detected | — | Detected | — |
| 264010.8_1_ | | uORF | uORF | | | | | | |
| 16:6760512 | | | | | | | | | |
| 0- | | | | | | | | | |
| 67644919:+ | | | | | | | | | |
| ENST00000 | EXTL3 | 5′ uORF | 5′ uORF | — | — | Detected | — | Detected | — |
| 220562.8_1_ | | | | | | | | | |
| 8:28573224- | | | | | | | | | |
| 28573419:+ | | | | | | | | | |
| ENST00000 | GOSR1 | 5′ uORF | 5′ uORF | Detected | — | Detected | — | Detected | — |
| 467337.6_1_ | | | | | | | | | |
| 17:2880824 | | | | | | | | | |
| 4- | | | | | | | | | |
| 28811254:+ | | | | | | | | | |
| ENST00000 | MCPH1-AS1 | Antisense | lncRNA | — | — | Detected | — | Detected | — |
| 515608.5_1_ | | | | | | | | | |
| 8:6484738- | | | | | | | | | |
| 6484828:- | | | | | | | | | |
| ENST00000 | PCNX4 | Out-of- | Out-of- | Detected | — | Detected | — | Detected | — |
| 391611.6_2_ | | Frame | Frame | | | | | | |
| 14:6057450 | | | | | | | | | |
| 8- | | | | | | | | | |
| 60574697:+ | | | | | | | | | |
| ENST00000 | RAB2A | Out-of- | Out-of- | Detected | — | Detected | — | Detected | — |
| 262646.11_1_ | | Frame | Frame | | | | | | |
| 8:6148466 | | | | | | | | | |
| 7- | | | | | | | | | |
| 61496833:+ | | | | | | | | | |
| ENST00000 | RNASE4 | 5′ uORF | 5′ uORF | — | — | Detected | — | Detected | — |
| 555835.2_2_ | | | | | | | | | |
| 14:2115277 | | | | | | | | | |
| 8- | | | | | | | | | |
| 21152838:+ | | | | | | | | | |
| ENST00000 | TBC1D23 | ncRNA | lncRNA | Detected | — | Detected | — | Detected | — |
| 484231.1_1_ | | Retained | | | | | | | |
| 3:10000059 | | Intron | | | | | | | |
| 5- | | | | | | | | | |
| 100000643: | | | | | | | | | |
| + | | | | | | | | | |
| ENST00000 | AZIN1 | 5′ uORF | 5′ uORF | Detected | — | — | Detected | Detected | — |
| 347770.8_1_ | | | | | | | | | |
| 8:10387027 | | | | | | | | | |
| 3- | | | | | | | | | |
| 103876088:- | | | | | | | | | |
| ENST00000 | SNX13 | 5′ uORF | 5′ uORF | Detected | — | — | Detected | Detected | — |
| 428135.7_1_ | | | | | | | | | |
| 7:17979950- | | | | | | | | | |
| 17980106:- | | | | | | | | | |
| ENST00000 | BTAF1 | 5′ uORF | 5′ uORF | Detected | — | — | — | — | Detected |
| 265990.10_1_ | | | | | | | | | |
| 10:936836 | | | | | | | | | |
| 79- | | | | | | | | | |
| 93683796:+ | | | | | | | | | |
| ENST00000 | BTD | ncRNA | lncRNA | — | — | — | — | — | Detected |
| 471964.5_1_ | | Processed | | | | | | | |
| 3:15674116- | | Transcript | | | | | | | |
| 15674227:+ | | | | | | | | | |
| ENST00000 | DIXDC1 | 5′ Overlap | 5′ Overlap | — | — | — | — | — | Detected |
| 618522.4_1_ | | uORF | uORF | | | | | | |
| 11:1118530 | | | | | | | | | |
| 07- | | | | | | | | | |
| 111889757: | | | | | | | | | |
| + | | | | | | | | | |
| ENST00000 | EIF2B3 | 5′ Overlap | 5′Ovelap | Detected | — | — | — | — | Detected |
| 620860.4_1_ | | uORF | uORF | | | | | | |
| 1:45446712- | | | | | | | | | |
| 45452201:- | | | | | | | | | |
| ENST00000 | FAM46A | 5′ Overlap | 5′ Overlap | — | — | — | — | — | Detected |
| 369754.7_2_ | | uORF | uORF | | | | | | |
| 6:82461791- | | | | | | | | | |
| 82462216:- | | | | | | | | | |
| ENST00000 | FAM65A | 5′ Overlap | 5′ Overlap | — | — | — | — | — | Detected |
| 540839.7_3_ | | uORF | uORF | | | | | | |
| 16:6756354 | | | | | | | | | |
| 7- | | | | | | | | | |
| 67572402:+ | | | | | | | | | |
| ENST00000 | FASTKD1 | 5′ uORF | 5′ uORF | Detected | — | — | — | — | Detected |
| 445210.1_1_ | | | | | | | | | |
| 2:17042858 | | | | | | | | | |
| 3- | | | | | | | | | |
| 170429512:- | | | | | | | | | |
| ENST00000 | GSN | 5′ Overlap | 5′ Overlap | — | — | — | — | — | Detected |
| 449733.7_1_ | | uORF | uORF | | | | | | |
| 9:12404472 | | | | | | | | | |
| 5- | | | | | | | | | |
| 124044779: | | | | | | | | | |
| + | | | | | | | | | |
| ENST00000 | HERPUD2 | 5′ uORF | 5′ uORF | — | — | — | — | — | Detected |
| 396081.5_1_ | | | | | | | | | |
| 7:35734046- | | | | | | | | | |
| 35734373:- | | | | | | | | | |
| ENST00000 | HIST1H3E | 5′ uORF | 5′ uORF | Detected | — | — | — | — | Detected |
| 634733.1_1_ | | | | | | | | | |
| 6:26224458- | | | | | | | | | |
| 26225162:+ | | | | | | | | | |
| ENST00000 | ITGAE | ncRNA | lncRNA | Detected | — | — | — | — | Detected |
| 572179.5_1_ | | Processed | | | | | | | |
| 17:3623679- | | Transcript | | | | | | | |
| 3627129:- | | | | | | | | | |
| ENST00000 | MAP7D3 | 5′ Overlap | 5′ Overlap | — | — | — | — | — | Detected |
| 316077.13_1_ | | uORF | uORF | | | | | | |
| X:1353335 | | | | | | | | | |
| 16- | | | | | | | | | |
| 135333558:- | | | | | | | | | |
| ENST00000 | MOB1B | 5′ Overlap | 5′ Overlap | Detected | — | — | — | — | Detected |
| 502869.5_2_ | | uORF | uORF | | | | | | |
| 4:71768157- | | | | | | | | | |
| 71808564:+ | | | | | | | | | |
| ENST00000 | MSRB3 | Out-of- | Out-of- | — | — | — | — | — | Detected |
| 535664.5_1_ | | Frame | Frame | | | | | | |
| 12:6572068 | | | | | | | | | |
| 6- | | | | | | | | | |
| 65847531:+ | | | | | | | | | |
| ENST00000 | MTIF3 | 5′ Overlap | 5′ Overlap | Detected | — | — | — | — | Detected |
| 381120.7_1_ | | uORF | uORF | | | | | | |
| 13:2801456 | | | | | | | | | |
| 9- | | | | | | | | | |
| 28024670:- | | | | | | | | | |
| ENST00000 | NBPF1 | 5′ uORF | 5′ uORF | — | — | — | — | — | Detected |
| 430580.6_1_ | | | | | | | | | |
| 1:16939836- | | | | | | | | | |
| 16939869:- | | | | | | | | | |
| ENST00000 | NCBP2 | 5′ Overlap | 5′ Overlap | — | — | — | — | — | Detected |
| 452404.6_1_ | | uORF | uORF | | | | | | |
| 3:19666630 | | | | | | | | | |
| 1- | | | | | | | | | |
| 196669112:- | | | | | | | | | |
| ENST00000 | OSBPL8 | Out-of- | Out-of- | Detected | — | — | — | — | Detected |
| 393249.6_1_ | | Frame | Frame | | | | | | |
| 12:7679365 | | | | | | | | | |
| 4- | | | | | | | | | |
| 76804396:- | | | | | | | | | |
| ENST00000 | PCF11 | TEC | Other | Detected | — | — | — | — | Detected |
| 624931.1_1_ | | | | | | | | | |
| 11:8286823 | | | | | | | | | |
| 8- | | | | | | | | | |
| 82868709:+ | | | | | | | | | |
| ENST00000 | PEX26 | 5′ uORF | 5′ uORF | Detected | — | — | — | — | Detected |
| 610387.4_2_ | | | | | | | | | |
| 22:1856076 | | | | | | | | | |
| 1- | | | | | | | | | |
| 18561111:+ | | | | | | | | | |
| ENST00000 | PMEL | 5′ Overlap | 5′ Overlap | — | — | — | — | — | Detected |
| 550590.5_1_ | | uORF | uORF | | | | | | |
| 12:5635214 | | | | | | | | | |
| 9- | | | | | | | | | |
| 56359825:- | | | | | | | | | |
| ENST00000 | POLR2K | 5′ uORF | 5′ uORF | Detected | — | — | — | — | Detected |
| 353107.7_1_ | | | | | | | | | |
| 8:10116284 | | | | | | | | | |
| 1- | | | | | | | | | |
| 101162934: | | | | | | | | | |
| + | | | | | | | | | |
| ENST00000 | PQBP1 | 5′ Overlap | 5′ Overlap | Detected | — | — | — | — | Detected |
| 473764.5_1_ | | uORF | uORF | | | | | | |
| X:48755552- | | | | | | | | | |
| 48755627:+ | | | | | | | | | |
| ENST00000 | PRKD3 | 5′ uORF | 5′ uORF | — | — | — | — | — | Detected |
| 379066.5_1_ | | | | | | | | | |
| 2:37544226- | | | | | | | | | |
| 37544932:- | | | | | | | | | |
| ENST00000 | PSME4 | Out-of- | Out-of- | Detected | — | — | — | — | Detected |
| 404125.5_1_ | | Frame | Frame | | | | | | |
| 2:54176390- | | | | | | | | | |
| 54176420:- | | | | | | | | | |
| ENST00000 | PTEN | 5′ uORF | 5′ uORF | Detected | — | — | — | — | Detected |
| 371953.7_1_ | | | | | | | | | |
| 10:8962346 | | | | | | | | | |
| 5- | | | | | | | | | |
| 89623600:+ | | | | | | | | | |
| ENST00000 | RNPS1 | ncRNA | lncRNA | Detected | — | — | — | — | Detected |
| 566783.1_1_ | | Retained | | | | | | | |
| 16:2317143- | | Intron | | | | | | | |
| 2317218:- | | | | | | | | | |
| ENST00000 | RP11-497E19.1 | lincRNA | lncRNA | — | — | — | — | — | Detected |
| 380722.2_1_ | | | | | | | | | |
| 14:8599606 | | | | | | | | | |
| 0- | | | | | | | | | |
| 85996315:- | | | | | | | | | |
| ENST00000 | RP11-705C15.2 | Pseudogene | Pseudogene | — | — | — | — | — | Detected |
| 633848.1_1_ | | | | | | | | | |
| 12:9808558- | | | | | | | | | |
| 9809555:+ | | | | | | | | | |
| ENST00000 | SCLT1 | 5′ uORF | 5′ uORF | — | — | — | — | — | Detected |
| 506368.5_2_ | | | | | | | | | |
| 4:13001434 | | | | | | | | | |
| 0- | | | | | | | | | |
| 130014460:- | | | | | | | | | |
| ENST00000 | SHOC2 | 5′ uORF | 5′ uORF | Detected | — | — | — | — | Detected |
| 369452.8_1_ | | | | | | | | | |
| 10:1127239 | | | | | | | | | |
| 43- | | | | | | | | | |
| 112724024: | | | | | | | | | |
| + | | | | | | | | | |
| ENST00000 | SLC7A8 | 5′ uORF | 5′ uORF | — | — | — | — | — | Detected |
| 316902.11_1_ | | | | | | | | | |
| 14:236521 | | | | | | | | | |
| 48- | | | | | | | | | |
| 23652238:- | | | | | | | | | |
| ENST00000 | SLX4 | 5′ uORF | 5′ uORF | — | — | — | — | — | Detected |
| 294008.3_1_ | | | | | | | | | |
| 16:3659441- | | | | | | | | | |
| 3659555:- | | | | | | | | | |
| ENST00000 | SNHG16 | lincRNA | lncRNA | Detected | — | — | — | — | Detected |
| 591956.1_1_ | | | | | | | | | |
| 17:7455387 | | | | | | | | | |
| 2- | | | | | | | | | |
| 74557410:+ | | | | | | | | | |
| ENST00000 | SPARC | 5′ Overlap | 5′ Overlap | Detected | — | — | — | — | Detected |
| 231061.8_1_ | | uORF | uORF | | | | | | |
| 5:15105421 | | | | | | | | | |
| 8- | | | | | | | | | |
| 151066527:- | | | | | | | | | |
| ENST00000 | SSH2 | 5′ Overlap | 5′ Overlap | — | — | — | — | — | Detected |
| 269033.7_1_ | | uORF | uORF | | | | | | |
| 17:2801164 | | | | | | | | | |
| 3- | | | | | | | | | |
| 28257046:- | | | | | | | | | |
| ENST00000 | TTN-AS1 | Antisense | lncRNA | Detected | — | — | — | — | Detected |
| 589434.5_1_ | | | | | | | | | |
| 2:17938814 | | | | | | | | | |
| 4- | | | | | | | | | |
| 179403504: | | | | | | | | | |
| + | | | | | | | | | |
| ENST00000 | TTTY15 | lincRNA | lncRNA | — | — | — | — | — | Detected |
| 440408.5_1_ | | | | | | | | | |
| Y:14798457- | | | | | | | | | |
| 14798529:+ | | | | | | | | | |
| ENST00000 | ZNF749 | 5′ Overlap | 5′ Overlap | — | — | — | — | — | Detected |
| 415248.1_1_ | | uORF | uORF | | | | | | |
| 19:5794691 | | | | | | | | | |
| 9- | | | | | | | | | |
| 57954712:+ | | | | | | | | | |
| ENST00000 | CCDC88A | 5′ uORF | 5′ uORF | Detected | Detected | — | — | — | Detected |
| 336838.10_1_ | | | | | | | | | |
| 2:5564627 | | | | | | | | | |
| 9- | | | | | | | | | |
| 55646603:- | | | | | | | | | |
| ENST00000 | SNRK | 5′ uORF | 5′ uORF | — | Detected | — | — | — | Detected |
| 296088.11_1_ | | | | | | | | | |
| 3:4334127 | | | | | | | | | |
| 4- | | | | | | | | | |
| 43344646:+ | | | | | | | | | |
| ENST00000 | TBL1XR1 | 5′ uORF | 5′ uORF | Detected | Detected | — | — | — | Detected |
| 424913.5_1_ | | | | | | | | | |
| 3:17677167 | | | | | | | | | |
| 3- | | | | | | | | | |
| 176816307:- | | | | | | | | | |
| ENST00000 | IFRD1 | 5′ uORF | 5′ uORF | Detected | — | — | Detected | — | Detected |
| 403825.7_1_ | | | | | | | | | |
| 7:11209054 | | | | | | | | | |
| 1- | | | | | | | | | |
| 112090697: | | | | | | | | | |
| + | | | | | | | | | |
| ENST00000 | PAICS | Out-of- | Out-of- | Detected | — | — | — | Detected | Detected |
| 264221.6_2_ | | Frame | Frame | | | | | | |
| 4:57307918- | | | | | | | | | |
| 57312926:+ | | | | | | | | | |
| ENST00000 | PLOD3 | 5′ uORF | 5′ uORF | Detected | — | — | — | Detected | Detected |
| 223127.7_2_ | | | | | | | | | |
| 7:10086069 | | | | | | | | | |
| 5- | | | | | | | | | |
| 100860860:- | | | | | | | | | |
| ENST00000 | PTPN13 | 5′ uORF | 5′ uORF | Detected | — | Detected | — | Detected | Detected |
| 427191.6_1_ | | | | | | | | | |
| 4:87515508- | | | | | | | | | |
| 87515601:+ | | | | | | | | | |
| ENST00000 | ADGRA2 | 5′ uORF | 5′ uORF | Detected | — | — | — | — | — |
| 315215.11_1_ | | | | | | | | | |
| 8:3765452 | | | | | | | | | |
| 7- | | | | | | | | | |
| 37654569:+ | | | | | | | | | |
| ENST00000 | ALKBH8 | 5′ uORF | 5′ uORF | Detected | — | — | — | — | — |
| 428149.6_1_ | | | | | | | | | |
| 11:1074363 | | | | | | | | | |
| 94- | | | | | | | | | |
| 107436448:- | | | | | | | | | |
| ENST00000 | ANKRD13A | ncRNA | lncRNA | — | — | — | — | — | — |
| 553251.5_1_ | | Retained | | | | | | | |
| 12:1104753 | | Intron | | | | | | | |
| 15- | | | | | | | | | |
| 110475375: | | | | | | | | | |
| + | | | | | | | | | |
| ENST00000 | ARAP1 | Out-of- | Out-of- | Detected | — | — | — | — | — |
| 429686.5_1_ | | Frame | Frame | | | | | | |
| 11:7242335 | | | | | | | | | |
| 8- | | | | | | | | | |
| 72423549:- | | | | | | | | | |
| ENST00000 | ARID1A | Out-of- | Out-of- | — | — | — | — | — | — |
| 430799.7_2_ | | Frame | Frame | | | | | | |
| 1:27056322- | | | | | | | | | |
| 27059215:+ | | | | | | | | | |
| ENST00000 | CPSF2 | 5′ uORF | 5′ uORF | Detected | — | — | — | — | — |
| 298875.8_1_ | | | | | | | | | |
| 14:9258837 | | | | | | | | | |
| 2- | | | | | | | | | |
| 92588465:+ | | | | | | | | | |
| ENST00000 | CTC-454I21.3 | 3′ dORF | 3′ dORF | — | — | — | — | — | — |
| 585860.2_3_ | | | | | | | | | |
| 19:3765657 | | | | | | | | | |
| 9- | | | | | | | | | |
| 37660760:- | | | | | | | | | |
| ENST00000 | EHBP1 | 5′ uORF | 5′ uORF | Detected | — | — | — | — | — |
| 431489.5_1_ | | | | | | | | | |
| 2:62932928- | | | | | | | | | |
| 62934072:+ | | | | | | | | | |
| ENST00000 | EIF2D | 5′ uORF | 5′ uORF | Detected | — | — | — | — | — |
| 367114.7_2_ | | | | | | | | | |
| 1:20678574 | | | | | | | | | |
| 1- | | | | | | | | | |
| 206785795:- | | | | | | | | | |
| ENST00000 | FAM20C | 5′ uORF | 5′ uORF | — | — | — | — | — | — |
| 313766.5_7: | | | | | | | | | |
| 193007- | | | | | | | | | |
| 193082:+ | | | | | | | | | |
| ENST00000 | GDE1 | 5′ Overlap | 5′ Overlap | — | — | — | — | — | — |
| 353258.7_1_ | | uORF | uORF | | | | | | |
| 16:1953322 | | | | | | | | | |
| 8- | | | | | | | | | |
| 19533372:- | | | | | | | | | |
| ENST00000 | HELZ2 | ncRNA | lncRNA | Detected | — | — | — | — | — |
| 479540.5_1_ | | Processed | | | | | | | |
| 20:6220408 | | Transcript | | | | | | | |
| 5- | | | | | | | | | |
| 62204322:- | | | | | | | | | |
| ENST00000 | HMGA1 | 5′ Overlap | 5′ Overlap | Detected | — | — | — | — | — |
| 401473.7_1_ | | uORF | uORF | | | | | | |
| 6:34204671- | | | | | | | | | |
| 34208558:+ | | | | | | | | | |
| ENST00000 | ITGB5 | Out-of- | Out-of- | — | — | — | — | — | — |
| 296181.8_1_ | | Frame | Frame | | | | | | |
| 3:12457825 | | | | | | | | | |
| 8- | | | | | | | | | |
| 124592335:- | | | | | | | | | |
| ENST00000 | LCP2 | ncRNA | lncRNA | — | — | — | — | — | — |
| 520322.1_1_ | | Retained | | | | | | | |
| 5:16967558 | | Intron | | | | | | | |
| 8- | | | | | | | | | |
| 169675669:- | | | | | | | | | |
| ENST00000 | LINC01415 | lincRNA | lncRNA | — | — | — | — | — | — |
| 588134.1_1_ | | | | | | | | | |
| 18:5344711 | | | | | | | | | |
| 6- | | | | | | | | | |
| 53447923:- | | | | | | | | | |
| ENST00000 | LURAP1L | 5′ uORF | 5′ uORF | Detected | — | — | — | — | — |
| 319264.3_1_ | | | | | | | | | |
| 9:12775157- | | | | | | | | | |
| 12775265:+ | | | | | | | | | |
| ENST00000 | MCAM | Out-of- | Out-of- | — | — | — | — | — | — |
| 264036.5_1_ | | Frame | Frame | | | | | | |
| 11:1191859 | | | | | | | | | |
| 54- | | | | | | | | | |
| 119187791:- | | | | | | | | | |
| ENST00000 | MEGF9 | 5′ uORF | 5′ uORF | Detected | — | — | — | — | — |
| 373930.3_1_ | | | | | | | | | |
| 9:12347665 | | | | | | | | | |
| 9- | | | | | | | | | |
| 123476740:- | | | | | | | | | |
| ENST00000 | MESDC1 | 5′ uORF | 5′ uORF | Detected | — | — | — | — | — |
| 267984.3_1_ | | | | | | | | | |
| 15:8129338 | | | | | | | | | |
| 5- | | | | | | | | | |
| 81293562:+ | | | | | | | | | |
| ENST00000 | MT1E | 3′ dORF | 3′ dORF | — | — | — | — | — | — |
| 306061.10_1_ | | | | | | | | | |
| 16:566603 | | | | | | | | | |
| 90- | | | | | | | | | |
| 56660912:+ | | | | | | | | | |
| ENST00000 | NEDD1 | Out-of- | Out-of- | Detected | — | — | — | — | — |
| 557644.5_1_ | | Frame | Frame | | | | | | |
| 12:9733044 | | | | | | | | | |
| 1- | | | | | | | | | |
| 97330501:+ | | | | | | | | | |
| ENST00000 | NR2F1 | 5′ uORF | 5′ uORF | — | — | — | — | — | — |
| 327111.7_1_ | | | | | | | | | |
| 5:92919776- | | | | | | | | | |
| 92920418:+ | | | | | | | | | |
| ENST00000 | NTN1 | 3′ dORF | 3′ dORF | — | — | — | — | — | — |
| 173229.6_1_ | | | | | | | | | |
| 17:9144472- | | | | | | | | | |
| 9144925:+ | | | | | | | | | |
| ENST00000 | OAT | nCRN | lncRNA | — | — | — | — | — | — |
| 476917.5_1_ | | Processed | | | | | | | |
| 10:1261005 | | Transcript | | | | | | | |
| 59- | | | | | | | | | |
| 126100658:- | | | | | | | | | |
| ENST00000 | PCIF1 | 5′ uORF | 5′ uORF | Detected | — | — | — | — | — |
| 372409.7_1_ | | | | | | | | | |
| 20:4456334 | | | | | | | | | |
| 6- | | | | | | | | | |
| 44566100:+ | | | | | | | | | |
| ENST00000 | PLP2 | 5′ Overlap | 5′ Overlap | Detected | — | — | — | — | — |
| 376327.5_1_ | | uORF | uORF | | | | | | |
| X:49028321- | | | | | | | | | |
| 49029491:+ | | | | | | | | | |
| ENST00000 | POSTN | Out-of- | Out-of- | — | — | — | — | — | — |
| 379742.4_1_ | | Frame | Frame | | | | | | |
| 13:3816626 | | | | | | | | | |
| 6- | | | | | | | | | |
| 38172793:- | | | | | | | | | |
| ENST00000 | RAB12 | 5′ Overlap | 5′ Overlap | Detected | — | — | — | — | — |
| 329286.6_1_ | | uORF | uORF | | | | | | |
| 18:8609717- | | | | | | | | | |
| 8609897:+ | | | | | | | | | |
| ENST00000 | RBCK1 | ncRNA | lncRNA | Detected | — | — | — | — | — |
| 465226.1_1_ | | Processed | | | | | | | |
| 20:388311- | | Transcript | | | | | | | |
| 389346:+ | | | | | | | | | |
| ENST00000 | RGMB | 5′ uORF | 5′ uORF | Detected | — | — | — | — | — |
| 513185.1_1_ | | | | | | | | | |
| 5:98109417- | | | | | | | | | |
| 98109651:+ | | | | | | | | | |
| ENST00000 | RINT1 | 5′ Overlap | 5′ Overlap | Detected | — | — | — | — | — |
| 257700.6_1_ | | uORF | uORF | | | | | | |
| 7:10517268 | | | | | | | | | |
| 2- | | | | | | | | | |
| 105177020: | | | | | | | | | |
| + | | | | | | | | | |
| ENST00000 | ROBO1 | 5′ uORF | 5′ uORF | Detected | — | — | — | — | — |
| 436010.6_1_ | | | | | | | | | |
| 3:79068249- | | | | | | | | | |
| 79068504:- | | | | | | | | | |
| ENST00000 | SELENOW | ncRNA | lncRNA | Detected | — | — | — | — | — |
| 598273.5_1_ | | Retained | | | | | | | |
| 19:4828414 | | Intron | | | | | | | |
| 5- | | | | | | | | | |
| 48284407:+ | | | | | | | | | |
| ENST00000 | SEPT5 | 5′ uORF | 5′ uORF | Detected | — | — | — | — | — |
| 455843.5_1_ | | | | | | | | | |
| 22:1970567 | | | | | | | | | |
| 9- | | | | | | | | | |
| 19706159:+ | | | | | | | | | |
| ENST00000 | SLC23A2 | 5′ uORF | 5′ uORF | Detected | — | — | — | — | — |
| 338244.5_1_ | | | | | | | | | |
| 20:4913325- | | | | | | | | | |
| 4913352:- | | | | | | | | | |
| ENST00000 | SLC25A37 | 5′ uORF | 5′ uORF | Detected | — | — | — | — | — |
| 519973.5_1_ | | | | | | | | | |
| 8:23386398- | | | | | | | | | |
| 23386443:+ | | | | | | | | | |
| ENST00000 | SNX6 | 5′ Overlap | 5′ Overlap | — | — | — | — | — | — |
| 396526.71 | | uORF | uORF | | | | | | |
| 14:3507484 | | | | | | | | | |
| 1- | | | | | | | | | |
| 35077276:- | | | | | | | | | |
| ENST00000 | SPG20 | 5′ Overlap | 5′ Overlap | Detected | — | — | — | — | — |
| 494062.2_1_ | | uORF | uORF | | | | | | |
| 13:3690987 | | | | | | | | | |
| 3- | | | | | | | | | |
| 36920564:- | | | | | | | | | |
| ENST00000 | SSBP3 | 5′ uORF | 5′ uORF | Detected | — | — | — | — | — |
| 610401.4_1_ | | | | | | | | | |
| 1:54871875- | | | | | | | | | |
| 54872031:- | | | | | | | | | |
| ENST00000 | TMEM140 | 5′ Overlap | 5′ Overlap | — | — | — | — | — | — |
| 275767.3_1_ | | uORF | uORF | | | | | | |
| 7:13483282 | | | | | | | | | |
| 9- | | | | | | | | | |
| 134849312: | | | | | | | | | |
| + | | | | | | | | | |
| ENST00000 | TTLL5 | Out-of- | Out-ofo | — | — | — | — | — | — |
| 557636.5_1_ | | Frame | Frame | | | | | | |
| 14:7624962 | | | | | | | | | |
| 2- | | | | | | | | | |
| 76249835:+ | | | | | | | | | |
| ENST00000 | TUBG2 | 5′ uORF | 5′ uORF | — | — | — | — | — | — |
| 251412.7_1_ | | | | | | | | | |
| 17:4081132 | | | | | | | | | |
| 9- | | | | | | | | | |
| 40811476:+ | | | | | | | | | |
| ENST00000 | WDTC1 | 3′ dORF | 3′ dORF | — | — | — | — | — | — |
| 319394.7_1_ | | | | | | | | | |
| 1:27633464- | | | | | | | | | |
| 27633656:+ | | | | | | | | | |
| ENST00000 | ZFYVE1 | 5′ uORF | 5′ uORF | Detected | — | — | — | — | — |
| 556143.5_1_ | | | | | | | | | |
| 14:7349139 | | | | | | | | | |
| 2- | | | | | | | | | |
| 73491458:- | | | | | | | | | |
| ENST00000 | ZNF677 | 3′ dORF | 3′ dORF | — | — | — | — | — | — |
| 599328.1_1_ | | | | | | | | | |
| 19:5374464 | | | | | | | | | |
| 9- | | | | | | | | | |
| 53744913:- | | | | | | | | | |
| ENST00000 | EMC6 | 5′ uORF | 5′ uORF | Detected | Detected | — | — | — | — |
| 397133.2_1_ | | | | | | | | | |
| 17:3572117- | | | | | | | | | |
| 3572156:+ | | | | | | | | | |
| ENST00000 | FAM69A | 5′ Overlap | 5′ Overlap | Detected | Detected | — | — | — | — |
| 559705.1_1_ | | uORF | uORF | | | | | | |
| 15:6438589 | | | | | | | | | |
| 9- | | | | | | | | | |
| 64386013:- | | | | | | | | | |
| ENST00000 | SMG1P3 | Pseudogene | Pseudogene | Detected | Detected | — | — | — | — |
| 522841.6_1_ | | | | | | | | | |
| 16:2148107 | | | | | | | | | |
| 5- | | | | | | | | | |
| 21531198:- | | | | | | | | | |
| ENST00000 | KLF6 | 5′ Overlap | 5′ Overlap | Detected | — | Detected | — | — | — |
| 497571.5_1_ | | uORF | uORF | | | | | | |
| 10:3824338- | | | | | | | | | |
| 3827372:- | | | | | | | | | |
| ENST00000 | RPL7A | ncRNA | lncRNA | Detected | — | — | — | Detected | — |
| 489392.1_1_ | | Processed | | | | | | | |
| 9:13621644 | | Transcript | | | | | | | |
| 8- | | | | | | | | | |
| 136216496: | | | | | | | | | |
| + | | | | | | | | | |
| ENST00000 | TMEM106C | 5′ Overlap | 5′ Overlap | — | — | — | — | Detected | — |
| 550146.5_1_ | | uORF | uORF | | | | | | |
| 12:4835737 | | | | | | | | | |
| 4- | | | | | | | | | |
| 48358078:+ | | | | | | | | | |
| ENST00000 | TPCN2 | Out-of- | Out-of- | — | — | — | — | Detected | — |
| 294309.7_2_ | | Frame | Frame | | | | | | |
| 11:6882152 | | | | | | | | | |
| 7- | | | | | | | | | |
| 68822650:+ | | | | | | | | | |
| ENST00000 | IKBKAP | 5′ Overlap | 5′ Overlap | Detected | — | Detected | — | Detected | — |
| 374647.9_1_ | | uORF | uORF | | | | | | |
| 9:11169334 | | | | | | | | | |
| 3- | | | | | | | | | |
| 111696326:- | | | | | | | | | |
| ENST00000 | PTGES3 | ncRNA | lncRNA | Detected | — | Detected | — | Detected | — |
| 537473.2_1_ | | Processed | | | | | | | |
| 12:5706554 | | Transcript | | | | | | | |
| 9- | | | | | | | | | |
| 57066549:- | | | | | | | | | |
| ENST00000 | TMEM168 | 5′ uORF | 5′ uORF | Detected | Detected | Detected | — | Detected | — |
| 312814.10_1_ | | | | | | | | | |
| 7:1124303 | | | | | | | | | |
| 70- | | | | | | | | | |
| 112430523:- | | | | | | | | | |
| ENST00000 | CCDC80 | 5′ uORF | 5′ uORF | — | — | Detected | — | — | Detected |
| 206423.7_2_ | | | | | | | | | |
| 3:11235940 | | | | | | | | | |
| 9- | | | | | | | | | |
| 112359550:- | | | | | | | | | |
| ENST00000 | AKAP6 | 5′ uORF | 5′ uORF | — | — | — | — | — | — |
| 554410.5_1_ | | | | | | | | | |
| 14:3296347 | | | | | | | | | |
| 9- | | | | | | | | | |
| 32963518:+ | | | | | | | | | |
| ENST00000 | AP2A2 | Out-of- | Out-of- | Detected | — | — | — | — | — |
| 448903.6_2_ | | Frame | Frame | | | | | | |
| 11:926043- | | | | | | | | | |
| 959460:+ | | | | | | | | | |
| ENST00000 | C6orf62 | 5′ uORF | 5′ uORF | Detected | — | — | — | — | — |
| 378119.8_1_ | | | | | | | | | |
| 6:24719560- | | | | | | | | | |
| 24720079:- | | | | | | | | | |
| ENST00000 | C7orf49 | 3′ dORF | 3′ dORF | — | — | — | — | — | — |
| 620897.4_2_ | | | | | | | | | |
| 7:13485109 | | | | | | | | | |
| 6- | | | | | | | | | |
| 134851354:- | | | | | | | | | |
| ENST00000 | CD59 | Out-of- | Out-of- | Detected | — | — | — | — | — |
| 395850.7_1_ | | Frame | Frame | | | | | | |
| 11:3373182 | | | | | | | | | |
| 0- | | | | | | | | | |
| 33743981:- | | | | | | | | | |
| ENST00000 | CES2 | 5′ uORF | 5′ uORF | — | — | — | — | — | — |
| 566182.1_1_ | | | | | | | | | |
| 16:6696843 | | | | | | | | | |
| 4- | | | | | | | | | |
| 66968817:+ | | | | | | | | | |
| ENST00000 | CLEC2B | ncRNA | lncRNA | Detected | — | — | — | — | — |
| 539028.1_1_ | | Retained | | | | | | | |
| 12:1001009 | | Intron | | | | | | | |
| 8- | | | | | | | | | |
| 10010200:- | | | | | | | | | |
| ENST00000 | CTC-205M6.1 | TEC | Other | — | — | — | — | — | — |
| 624602.1_1_ | | | | | | | | | |
| 5:18023750 | | | | | | | | | |
| 7- | | | | | | | | | |
| 180237573: | | | | | | | | | |
| + | | | | | | | | | |
| ENST00000 | CTNNB1 | 5′ Overlap | 5′ Overlap | — | — | — | — | — | — |
| 453024.5_1_ | | uORF | uORF | | | | | | |
| 3:41241021- | | | | | | | | | |
| 41265725:+ | | | | | | | | | |
| ENST00000 | DCAF16 | Out-of- | Out-of- | Detected | — | — | — | — | — |
| 382247.5_1_ | | Frame | Frame | | | | | | |
| 4:17805583- | | | | | | | | | |
| 17805688:- | | | | | | | | | |
| ENST00000 | EFTUD2 | ncRNA | lncRNA | — | — | — | — | — | — |
| 590105.1_1_ | | Retained | | | | | | | |
| 17:4297178 | | Intron | | | | | | | |
| 0- | | | | | | | | | |
| 42971870:- | | | | | | | | | |
| ENST00000 | EIF1 | 3′Overlap | 3′Overlap | Detected | — | — | — | — | — |
| 462917.1_1_ | | dORF | dORF | | | | | | |
| 17:3984712 | | | | | | | | | |
| 0- | | | | | | | | | |
| 39847177:+ | | | | | | | | | |
| ENST00000 | EIF5 | 5′ Overlap | 5′ Overlap | — | — | — | — | — | — |
| 560763.5_2_ | | uORF | uORF | | | | | | |
| 14:1038005 | | | | | | | | | |
| 97- | | | | | | | | | |
| 103802258: | | | | | | | | | |
| + | | | | | | | | | |
| ENST00000 | EMSY | 5′ Overlap | 5′ Overlap | Detected | — | — | — | — | — |
| 427574.6_1_ | | uORF | uORF | | | | | | |
| 11:7615614 | | | | | | | | | |
| 9- | | | | | | | | | |
| 76164398:+ | | | | | | | | | |
| ENST00000 | ESCO1 | 5′ uORF | 5′ uORF | Detected | — | — | — | — | — |
| 269214.9_1_ | | | | | | | | | |
| 18:1915518 | | | | | | | | | |
| 3- | | | | | | | | | |
| 19155270:- | | | | | | | | | |
| ENST00000 | FAM204A | Out-of- | Out-of- | Detected | — | — | — | — | — |
| 369183.8_1_ | | Frame | Frame | | | | | | |
| 10:1200957 | | | | | | | | | |
| 85- | | | | | | | | | |
| 120095896:- | | | | | | | | | |
| ENST00000 | FANCF | Out-of- | Out-of- | — | — | — | — | — | — |
| 327470.4_1_ | | Frame | Frame | | | | | | |
| 11:2264711 | | | | | | | | | |
| 5- | | | | | | | | | |
| 22647214:- | | | | | | | | | |
| ENST00000 | FGD-AS1 | Antisense | lncRNA | Detected | — | — | — | — | — |
| 424349.1_1_ | | | | | | | | | |
| 3:14987427- | | | | | | | | | |
| 14987592:- | | | | | | | | | |
| 1 | | | | | | | | | |
| ENST00000 | HCG18 | Antisense | lncRNA | Detected | — | — | — | — | — |
| 454129.5_1_ | | | | | | | | | |
| 6:30260032- | | | | | | | | | |
| 30260266:- | | | | | | | | | |
| ENST00000 | IFT81 | Out-of- | Out-of- | — | — | — | — | — | — |
| 552912.5_1_ | | Frame | Frame | | | | | | |
| 12:1106183 | | | | | | | | | |
| 29- | | | | | | | | | |
| 110628779: | | | | | | | | | |
| + | | | | | | | | | |
| ENST00000 | ITGB1 | Out-of- | Out-of- | — | — | — | — | — | — |
| 488494.5_1_ | | Frame | Frame | | | | | | |
| 10:3322143 | | | | | | | | | |
| 7- | | | | | | | | | |
| 33221518:- | | | | | | | | | |
| ENST00000 | LINGO1 | 5′ Overlap | 5′ Overlap | — | — | — | — | — | — |
| 355300.6_1_ | | uORF | uORF | | | | | | |
| 15:7790824 | | | | | | | | | |
| 1- | | | | | | | | | |
| 77924773:- | | | | | | | | | |
| ENST00000 | MED21 | Out-of- | Out-of- | Detected | — | — | — | — | — |
| 282892.3_1_ | | Frame | Frame | | | | | | |
| 12:2717935 | | | | | | | | | |
| 9- | | | | | | | | | |
| 27180306:+ | | | | | | | | | |
| ENST00000 | MTCO2P22 | Pseudogene | Pseudogene | — | — | — | — | — | — |
| 603246.1_1_ | | | | | | | | | |
| 02 | | | | | | | | | |
| 5:99389051- | | | | | | | | | |
| 99389243:- | | | | | | | | | |
| ENST00000 | NFX1 | Out-of- | Out-of- | Detected | — | — | — | — | — |
| 318524.6_1_ | | Frame | Frame | | | | | | |
| 9:33294912- | | | | | | | | | |
| 33295365:+ | | | | | | | | | |
| ENST00000 | NIPA2 | 5′ uORF | 5′ uORF | Detected | — | — | — | — | — |
| 337451.7_1_ | | | | | | | | | |
| 15:2303420 | | | | | | | | | |
| 9- | | | | | | | | | |
| 23034353:- | | | | | | | | | |
| ENST00000 | NLGN1 | 5′ uORF | 5′ uORF | — | — | — | — | — | — |
| 423427.1_2_ | | | | | | | | | |
| 3:17332214 | | | | | | | | | |
| 8- | | | | | | | | | |
| 173322187: | | | | | | | | | |
| + | | | | | | | | | |
| ENST00000 | PHACTR1 | Out-of- | Out-of- | Detected | — | — | — | — | — |
| 332995.11_1_ | | Frame | Frame | | | | | | |
| 6:1328371 | | | | | | | | | |
| 1- | | | | | | | | | |
| 13286435:+ | | | | | | | | | |
| ENST00000 | PPIP5K2 | 5′ uORF | 5′ uORF | Detected | — | — | — | — | — |
| 613674.4_1_ | | | | | | | | | |
| 5:10246506 | | | | | | | | | |
| 4- | | | | | | | | | |
| 102465157: | | | | | | | | | |
| + | | | | | | | | | |
| ENST00000 | RAD17 | 5′ uORF | 5′ uORF | — | — | — | — | — | — |
| 345306.10_2_ | | | | | | | | | |
| 5:6866568 | | | | | | | | | |
| 5- | | | | | | | | | |
| 68667980:+ | | | | | | | | | |
| ENST00000 | RBM20 | ncRNA | lncRNA | — | — | — | — | — | — |
| 471172.1_1_ | | Processed | | | | | | | |
| 10:1125908 | | Transcript | | | | | | | |
| 20- | | | | | | | | | |
| 112591150: | | | | | | | | | |
| + | | | | | | | | | |
| ENST00000 | RP-11- | lincRNA | lncRNA | Detected | — | — | — | — | — |
| 530759.1_1_ | 111M22.3 | | | | | | | | |
| 11:7615533 | | | | | | | | | |
| 8- | | | | | | | | | |
| 76155506:- | | | | | | | | | |
| ENST00000 | RP-11-726G1.1 | Out-of- | Out-of- | — | — | — | — | — | — |
| 640129.1_1_ | | Frame | Frame | | | | | | |
| 12:9723336- | | | | | | | | | |
| 9723429:+ | | | | | | | | | |
| 1.1 | | | | | | | | | |
| ENST00000 | RPL36AP21 | Pseudogene | Pseudogene | — | — | — | — | — | — |
| 639134.1_1_ | | | | | | | | | |
| 5:18049650- | | | | | | | | | |
| 18049725:+ | | | | | | | | | |
| ENST00000 | RSRC2 | 3′ dORF | 3′ dORF | — | — | — | — | — | — |
| 532695.5_1_ | | | | | | | | | |
| 12:1230050 | | | | | | | | | |
| 70- | | | | | | | | | |
| 123006782:- | | | | | | | | | |
| ENST00000 | SNRNP25 | 5′ Overlap | 5′ Overlap | Detected | — | — | — | — | — |
| 383018.7_1_ | | uORF | uORF | | | | | | |
| 16:103936- | | | | | | | | | |
| 105489:+ | | | | | | | | | |
| ENST00000 | SOCS6 | Out-of- | Out-of- | — | — | — | — | — | — |
| 397942.3_1_ | | Frame | Frame | | | | | | |
| 18:6799204 | | | | | | | | | |
| 9- | | | | | | | | | |
| 67992124:+ | | | | | | | | | |
| ENST00000 | SYTL2 | 5′ uORF | 5′ uORF | — | — | — | — | — | — |
| 532995.5_1_ | | | | | | | | | |
| 11:8542998 | | | | | | | | | |
| 9- | | | | | | | | | |
| 85430118:- | | | | | | | | | |
| ENST00000 | TCAIM | 5′ Overlap | 5′ Overlap | Detected | — | — | — | — | — |
| 383746.7_2_ | | uORF | uORF | | | | | | |
| 3:44396236- | | | | | | | | | |
| 44396287:+ | | | | | | | | | |
| ENST00000 | TDRD32 | 5′ uORF | 5′ uORF | — | — | — | — | — | — |
| 196169.7_2_ | | | | | | | | | |
| 13:6097111 | | | | | | | | | |
| 6- | | | | | | | | | |
| 61013880:+ | | | | | | | | | |
| ENST00000 | TEN1 | 5′ uORF | 5′ uORF | Detected | — | — | — | — | — |
| 397640.5_1_ | | | | | | | | | |
| 17:7397533 | | | | | | | | | |
| 4- | | | | | | | | | |
| 73975502:+ | | | | | | | | | |
| ENST00000 | YEATS2 | 5′ Overlap | 5′ Overlap | Detected | — | — | — | — | — |
| 305135.9_1_ | | uORF | uORF | | | | | | |
| 3:18343294 | | | | | | | | | |
| 2- | | | | | | | | | |
| 183435501: | | | | | | | | | |
| + | | | | | | | | | |
| ENST00000 | ZBED4 | 5′ uORF | 5′ uORF | — | — | — | — | — | — |
| 216268.5_1_ | | | | | | | | | |
| 22:5027698 | | | | | | | | | |
| 4- | | | | | | | | | |
| 50277221:+ | | | | | | | | | |
| ENST00000 | ZHX1 | 5′ Overlap | 5′ Overlap | Detected | — | — | — | — | — |
| 297857.3_1_ | | uORF | uORF | | | | | | |
| 8:12426833 | | | | | | | | | |
| 9- | | | | | | | | | |
| 124279533:- | | | | | | | | | |
| ENST00000 | ZNF146 | 5′ uORF | 5′ uORF | — | — | — | — | — | — |
| 456324.5_1_ | | | | | | | | | |
| 19:3672691 | | | | | | | | | |
| 1- | | | | | | | | | |
| 36727052:+ | | | | | | | | | |
| TCONS_00 | linc-CDYL-1 | lincRNA | lncRNA | — | — | — | — | — | — |
| 011153_6:4 | | | | | | | | | |
| 611322- | | | | | | | | | |
| 4611508:+ | | | | | | | | | |
| T196935_2: | nan | lincRNA | lncRNA | — | — | — | — | — | — |
| 113299507- | | | | | | | | | |
| 113299648:- | | | | | | | | | |
| ENST00000 | ATRAID | 5′ Overlap | 5′ Overlap | Detected | Detected | — | — | — | Detected |
| 405489.7_2_ | | uORF | uORF | | | | | | |
| 2:27435269- | | | | | | | | | |
| 27435350:+ | | | | | | | | | |
| ENST00000 | ACAA2 | 5′ Overlap | 5′ Overlap | Detected | — | — | — | — | — |
| 285093.14_1_ | | uORF | uORF | | | | | | |
| 18:473401 | | | | | | | | | |
| 24- | | | | | | | | | |
| 47340193:- | | | | | | | | | |
| ENST00000 | ATF5 | 5′ Overlap | 5′ Overlap | — | — | — | — | — | — |
| 597227.5_1_ | | uORF | uORF | | | | | | |
| 19:5043251 | | | | | | | | | |
| 4- | | | | | | | | | |
| 50434160:+ | | | | | | | | | |
| ENST00000 | ATG5 | Out-of- | Out-of- | Detected | — | — | — | — | — |
| 613993.1_2_ | | Frame | Frame | | | | | | |
| 6:10676400 | | | | | | | | | |
| 1- | | | | | | | | | |
| 106764076:- | | | | | | | | | |
| ENST00000 | AZI2 | 5′ uORF | 5′ uORF | Detected | — | — | — | — | — |
| 479665.5_2_ | | | | | | | | | |
| 3:28390190- | | | | | | | | | |
| 28390412:- | | | | | | | | | |
| ENST00000 | C4orf3 | 3′Overlap | 3′Overlap | — | — | — | — | — | — |
| 399075.6_1_ | | dORF | dORF | | | | | | |
| 4:12021988 | | | | | | | | | |
| 6- | | | | | | | | | |
| 120221493:- | | | | | | | | | |
| ENST00000 | CALHM2 | ncRNA | lncRNA | Detected | — | — | — | — | — |
| 461631.1_1_ | | Processed | | | | | | | |
| 10:1052105 | | Transcript | | | | | | | |
| 16- | | | | | | | | | |
| 105210915:- | | | | | | | | | |
| ENST00000 | CD36 | 5′ uORF | 5′ uORF | — | — | — | — | — | — |
| 482059.6_1_ | | | | | | | | | |
| 7:80267993- | | | | | | | | | |
| 80269165:+ | | | | | | | | | |
| ENST00000 | CDC42BPA | 5′ uORF | 5′ uORF | — | — | — | — | — | — |
| 366769.7_1_ | | | | | | | | | |
| 1:22750557 | | | | | | | | | |
| 0- | | | | | | | | | |
| 227505621:- | | | | | | | | | |
| ENST00000 | CDH13 | 5′ Overlap | 5′ Overlap | — | — | — | — | — | — |
| 567109.5_1_ | | uORF | uORF | | | | | | |
| 16:8266058 | | | | | | | | | |
| 3- | | | | | | | | | |
| 82660685:+ | | | | | | | | | |
| ENST00000 | FGF7 | 5′ uORF | 5′ uORF | — | — | — | — | — | — |
| 267843.8_1_ | | | | | | | | | |
| 15:4971645 | | | | | | | | | |
| 9- | | | | | | | | | |
| 49716492:+ | | | | | | | | | |
| ENST00000 | HAS2 | 5′ uORF | 5′ uORF | — | — | — | — | — | — |
| 303924.4_1_ | | | | | | | | | |
| 8:12265311 | | | | | | | | | |
| 1- | | | | | | | | | |
| 122653336:- | | | | | | | | | |
| ENST00000 | HNRNPA1P30 | Pseudogene | Pseudogene | — | — | — | — | — | — |
| 440699.1_1_ | | | | | | | | | |
| 13:2152363 | | | | | | | | | |
| 0- | | | | | | | | | |
| 21523738:- | | | | | | | | | |
| ENST00000 | HPS5 | 5′ Overlap | 5′ Overlap | — | — | — | — | — | — |
| 396253.7_1_ | | uORF | uORF | | | | | | |
| 11:1833356 | | | | | | | | | |
| 1- | | | | | | | | | |
| 18343686:- | | | | | | | | | |
| ENST00000 | IGFBP5 | 5′ uORF | 5′ uORF | — | — | — | — | — | — |
| 233813.4_1_ | | | | | | | | | |
| 2:21755950 | | | | | | | | | |
| 7- | | | | | | | | | |
| 217559828:- | | | | | | | | | |
| ENST00000 | KDELR1 | 5′ Overlap | 5′ Overlap | — | — | — | — | — | — |
| 597017.5_1_ | | uORF | uORF | | | | | | |
| 19:4889292 | | | | | | | | | |
| 4- | | | | | | | | | |
| 48893852:- | | | | | | | | | |
| ENST00000 | LAMA4 | 5′ Overlap | 5′ Overlap | — | — | — | — | — | — |
| 230538.11_1_ | | uORF | uORF | | | | | | |
| 6:1125756 | | | | | | | | | |
| 72- | | | | | | | | | |
| 112575810:- | | | | | | | | | |
| ENST00000 | LINC00674 | Pseudogene | Pseudogene | — | — | — | — | — | — |
| 435469.2_1_ | | | | | | | | | |
| 17:6609917 | | | | | | | | | |
| 1- | | | | | | | | | |
| 66110645:+ | | | | | | | | | |
| ENST00000 | LOXL3 | ncRNA | lncRNA | Detected | — | — | — | — | — |
| 470907.6_1_ | | Retained | | | | | | | |
| 2:74763585- | | Intron | | | | | | | |
| 74776752:- | | | | | | | | | |
| ENST00000 | MACF1 | 5′ uORF | 5′ uORF | — | — | — | — | — | — |
| 564288.5_2_ | | | | | | | | | |
| 1:39670537- | | | | | | | | | |
| 39670564:+ | | | | | | | | | |
| ENST00000 | MKKS | 5′ Overlap | 5′ Overlap | Detected | — | — | — | — | — |
| 347364.7_1_ | | uORF | uORF | | | | | | |
| 20:1040126 | | | | | | | | | |
| 9- | | | | | | | | | |
| 10414870:- | | | | | | | | | |
| ENST00000 | MMP2 | 5′ Overlap | 5′ Overlap | — | — | — | — | — | — |
| 219070.8_1_ | | uORF | uORF | | | | | | |
| 16:5551329 | | | | | | | | | |
| 6- | | | | | | | | | |
| 55513404:+ | | | | | | | | | |
| ENST00000 | NEDD8 | ncRNA | lncRNA | Detected | — | — | — | — | — |
| 527046.5_1_ | | Processed | | | | | | | |
| 14:2468639 | | Transcript | | | | | | | |
| 1- | | | | | | | | | |
| 24686427:- | | | | | | | | | |
| ENST00000 | NEK9 | 5′ uORF | 5′ uORF | — | — | — | — | — | — |
| 557673.5_3_ | | | | | | | | | |
| 14:7559088 | | | | | | | | | |
| 9- | | | | | | | | | |
| 75593687:- | | | | | | | | | |
| ENST00000 | NOTCH2NL | Out-of- | Out-of- | — | — | — | — | — | — |
| 578207.5_1_ | | Frame | Frame | | | | | | |
| 1:14524884 | | | | | | | | | |
| 8- | | | | | | | | | |
| 145273378: | | | | | | | | | |
| + | | | | | | | | | |
| ENST00000 | PPP6R3 | 3′Overlap | 3′Overlap | — | — | — | — | — | — |
| 526593.1_1_ | | dORF | dORF | | | | | | |
| 11:6837705 | | | | | | | | | |
| 5- | | | | | | | | | |
| 68380582:+ | | | | | | | | | |
| ENST00000 | RNF31 | 5′ Overlap | 5′ Overlap | — | — | — | — | — | — |
| 558634.5_1_ | | uORF | uORF | | | | | | |
| 14:2461596 | | | | | | | | | |
| 1- | | | | | | | | | |
| 24617291:+ | | | | | | | | | |
| ENST00000 | RPS8 | Out-of- | Out-of- | Detected | — | — | — | — | — |
| 372209.3_1_ | | Frame | Frame | | | | | | |
| 1:45241798- | | | | | | | | | |
| 45243366:+ | | | | | | | | | |
| ENST00000 | SGMS1 | 5′ uORF | 5′ uORF | Detected | — | — | — | — | — |
| 429490.5_1_ | | | | | | | | | |
| 10:5219328 | | | | | | | | | |
| 0- | | | | | | | | | |
| 52220465:- | | | | | | | | | |
| ENST00000 | SIPA1L3 | 5′ uORF | 5′ uORF | Detected | — | — | — | — | — |
| 222345.10_1_ | | | | | | | | | |
| 19:385719 | | | | | | | | | |
| 27- | | | | | | | | | |
| 38572134:+ | | | | | | | | | |
| ENST00000 | TULP4 | 5′ uORF | 5′ uORF | — | — | — | — | — | — |
| 367097.7_1_ | | | | | | | | | |
| 6:15873486 | | | | | | | | | |
| 7- | | | | | | | | | |
| 158734906: | | | | | | | | | |
| + | | | | | | | | | |
| ENST00000 | TXNRD1 | 5′ uORF | 5′ uORF | — | — | — | — | — | — |
| 354940.10_1_ | | | | | | | | | |
| 12:104680 | | | | | | | | | |
| 894- | | | | | | | | | |
| 104681023: | | | | | | | | | |
| + | | | | | | | | | |
| ENST00000 | VDAC2 | 5′ Overlap | 5′ Overlap | Detected | — | — | — | — | — |
| 332211.10_2_ | | uORF | uORF | | | | | | |
| 10:769705 | | | | | | | | | |
| 48- | | | | | | | | | |
| 76973790:+ | | | | | | | | | |
| ENST00000 | ZMAT3 | 5′ Overlap | 5′ Overlap | Detected | — | — | — | — | — |
| 311417.6_1_ | | uORF | uORF | | | | | | |
| 3:17878553 | | | | | | | | | |
| 6- | | | | | | | | | |
| 178789480:- | | | | | | | | | |
| ENST00000 | ZNF319 | 5′ uORF | 5′ uORF | Detected | — | — | — | — | — |
| 299237.2_1_ | | | | | | | | | |
| 16:5803356 | | | | | | | | | |
| 8- | | | | | | | | | |
| 58033697:- | | | | | | | | | |
| ENST00000 | ZPBP2 | Out-of- | Out-of- | — | — | — | — | — | — |
| 584588.5_1_ | | Frame | Frame | | | | | | |
| 17:3802778 | | | | | | | | | |
| 2- | | | | | | | | | |
| 38031519:+ | | | | | | | | | |
| ENST00000 | COL11A1 | 5′ uORF | 5′ uORF | — | — | Detected | — | — | — |
| 370096.7_1_ | | | | | | | | | |
| 1:10357400 | | | | | | | | | |
| 0- | | | | | | | | | |
| 103574030:- | | | | | | | | | |
| ENST00000 | XPR1 | 5′ Overlap | 5′ Overlap | Detected | Detected | — | — | — | Detected |
| 367590.8_1_ | | uORF | uORF | | | | | | |
| 1:18060119 | | | | | | | | | |
| 2- | | | | | | | | | |
| 180601402: | | | | | | | | | |
| + | | | | | | | | | |
| ENST00000 | SAT1 | 5′ Overlap | 5′ Overlap | Detected | — | Detected | — | — | — |
| 379251.7_1_ | | uORF | uORF | | | | | | |
| X:23801394- | | | | | | | | | |
| 2380148 1 :+ | | | | | | | | | |
| ENST00000 | TOAK3 | 5′ uORF | 5′ uORF | Detected | — | Detected | — | — | — |
| 419821.6_1_ | | | | | | | | | |
| 12:1186934 | | | | | | | | | |
| 56- | | | | | | | | | |
| 118704503:- | | | | | | | | | |
| ENST00000 | EIF4G2 | ncRNA | lncRNA | Detected | — | — | — | — | — |
| 532383.5_1_ | | Retained | | | | | | | |
| 11:1082507 | | Intron | | | | | | | |
| 0- | | | | | | | | | |
| 10825157:- | | | | | | | | | |
| ENST00000 | GABARAP | 3′ dORF | 3′ dORF | Detected | — | — | — | — | Detected |
| 571129.5_1_ | | | | | | | | | |
| 17:7144048- | | | | | | | | | |
| 7144743:- | | | | | | | | | |
| ENST00000 | AKT1S1 | 5′ uORF | 5′ uORF | — | — | — | — | — | — |
| 391832.7_1_ | | | | | | | | | |
| 19:5038039 | | | | | | | | | |
| 4- | | | | | | | | | |
| 50380442:- | | | | | | | | | |
| ENST00000 | AP000525.9 | ncRNA | lncRNA | Detected | — | — | — | — | — |
| 383038.3_22: | | Processed | | | | | | | |
| 16189326- | | Transcript | | | | | | | |
| 16190688:- | | | | | | | | | |
| ENST00000 | BAMBI | Out-of- | Out-of- | Detected | — | — | — | — | — |
| 375533.5_1_ | | Frame | Frame | | | | | | |
| 10:2897027 | | | | | | | | | |
| 9- | | | | | | | | | |
| 28970974:+ | | | | | | | | | |
| ENST00000 | BACM | 3′ dORF | 3′ dORF | — | — | — | — | — | — |
| 611077.4_1_ | | | | | | | | | |
| 19:4532319 | | | | | | | | | |
| 6- | | | | | | | | | |
| 45323622:+ | | | | | | | | | |
| ENST00000 | CALM2 | Out-of- | Out-of- | Detected | — | — | — | — | — |
| 456319.5_2_ | | Frame | Frame | | | | | | |
| 2:47397898- | | | | | | | | | |
| 47403621:- | | | | | | | | | |
| ENST00000 | CENPBD1P1 | Pseudogene | Pseudogene | — | — | — | — | — | — |
| 487264.5_1_ | | | | | | | | | |
| 19:5908684 | | | | | | | | | |
| 1- | | | | | | | | | |
| 59086880:+ | | | | | | | | | |
| ENST00000 | CENPN | 5′ Overlap | 5′ Overlap | — | — | — | — | — | — |
| 305850.9_1_ | | uORF | uORF | | | | | | |
| 16:8104085 | | | | | | | | | |
| 7- | | | | | | | | | |
| 81045584:+ | | | | | | | | | |
| ENST00000 | CHST6 | 5′ uORF | 5′ uORF | — | — | — | — | — | — |
| 390664.2_1_ | | | | | | | | | |
| 16:7551577 | | | | | | | | | |
| 4- | | | | | | | | | |
| 75524658:- | | | | | | | | | |
| ENST00000 | CLIP1 | 5′ uORF | 5′ uORF | — | — | — | — | — | — |
| 361654.8_3_ | | | | | | | | | |
| 12:1228650 | | | | | | | | | |
| 79- | | | | | | | | | |
| 122907104:- | | | | | | | | | |
| ENST00000 | COA1 | 5′ Overlap | 5′ Overlap | — | — | — | — | — | — |
| 395879.5_2_ | | uORF | uORF | | | | | | |
| 7:43688212- | | | | | | | | | |
| 43688257:- | | | | | | | | | |
| ENST00000 | CPED1 | 5′ uORF | 5′ uORF | — | — | — | — | — | — |
| 310396.9_1_ | | | | | | | | | |
| 7:12062892 | | | | | | | | | |
| 5- | | | | | | | | | |
| 120629484: | | | | | | | | | |
| + | | | | | | | | | |
| ENST00000 | CRYAB | 5′ uORF | 5′ uORF | — | — | — | — | — | — |
| 526180.5_2_ | | | | | | | | | |
| 11:1117825 | | | | | | | | | |
| 75- | | | | | | | | | |
| 111782635:- | | | | | | | | | |
| ENST00000 | CTLA4 | 5′ Overlap | 5′ Overlap | — | — | — | — | — | — |
| 302823.7_1_ | | uORF | uORF | | | | | | |
| 2:20473260 | | | | | | | | | |
| 6- | | | | | | | | | |
| 204732705: | | | | | | | | | |
| + | | | | | | | | | |
| ENST00000 | CTNND1 | 5′ Overlap | 5′ Overlap | — | — | — | — | — | — |
| 532463.5_1_ | | uORF | uORF | | | | | | |
| 11:5752952 | | | | | | | | | |
| 9- | | | | | | | | | |
| 57561533:+ | | | | | | | | | |
| ENST00000 | DAGLA | 5′ uORF | 5′ uORF | — | — | — | — | — | — |
| 257215.9_1_ | | | | | | | | | |
| 11:6144792 | | | | | | | | | |
| 7- | | | | | | | | | |
| 61487603:+ | | | | | | | | | |
| ENST00000 | DCP2 | 5′ Overlap | 5′ Overlap | Detected | — | — | — | — | — |
| 504961.1_1_ | | uORF | uORF | | | | | | |
| 5:11231260 | | | | | | | | | |
| 1- | | | | | | | | | |
| 112319690: | | | | | | | | | |
| + | | | | | | | | | |
| ENST00000 | DCPS | Out-of- | Out-of- | Detected | — | — | — | — | — |
| 263579.4_1_ | | Frame | Frame | | | | | | |
| 11:1261764 | | | | | | | | | |
| 68- | | | | | | | | | |
| 126176510: | | | | | | | | | |
| + | | | | | | | | | |
| ENST00000 | DUSP18 | 5′ uORF | 5′ uORF | Detected | — | — | — | — | — |
| 377087.3_1_ | | | | | | | | | |
| 22:3106000 | | | | | | | | | |
| 9- | | | | | | | | | |
| 31063612:- | | | | | | | | | |
| ENST00000 | EHBP1 | 5′ uORF | 5′ uORF | — | — | — | — | — | — |
| 405015.7_1_ | | | | | | | | | |
| 2:62934186- | | | | | | | | | |
| 62934213:+ | | | | | | | | | |
| ENST00000 | EIF1B | ncRNA | lncRNA | — | — | — | — | — | — |
| 487151.1_1_ | | Retained | | | | | | | |
| 3:40352488- | | Intron | | | | | | | |
| 40352533:+ | | | | | | | | | |
| ENST00000 | EIF1 | Out-of- | Out-of- | Detected | — | — | — | — | — |
| 469257.1_1_ | | Frame | Frame | | | | | | |
| 17:3984527 | | | | | | | | | |
| 6- | | | | | | | | | |
| 39846098:+ | | | | | | | | | |
| ENST00000 | ELP2 | Out-of- | Out-of- | — | — | — | — | — | — |
| 423854.6_1_ | | Frame | Frame | | | | | | |
| 18:3372281 | | | | | | | | | |
| 0- | | | | | | | | | |
| 33722882:+ | | | | | | | | | |
| ENST00000 | ENTPD6 | 5′ Overlap | 5′ Overlap | Detected | — | — | — | — | — |
| 360031.6_2_ | | uORF | uORF | | | | | | |
| 20:2517643 | | | | | | | | | |
| 9- | | | | | | | | | |
| 25187192:+ | | | | | | | | | |
| ENST00000 | FANCB | 5′ uORF | 5′ uORF | — | — | — | — | — | — |
| 398334.5_2_ | | | | | | | | | |
| X:14887065- | | | | | | | | | |
| 14891178:- | | | | | | | | | |
| ENST00000 | FCF1 | 5′ Overlap | 5′ Overlap | Detected | — | — | — | — | — |
| 534938.6_2_ | | uORF | uORF | | | | | | |
| 14:7517989 | | | | | | | | | |
| 4- | | | | | | | | | |
| 75180224:+ | | | | | | | | | |
| ENST00000 | FCGRT | 3′ dORF | 3′ dORF | Detected | — | — | — | — | — |
| 598319.5_1_ | | | | | | | | | |
| 19:5002790 | | | | | | | | | |
| 1- | | | | | | | | | |
| 50027952:+ | | | | | | | | | |
| ENST00000 | FDPS | Out-of- | Out-of- | Detected | — | — | — | — | — |
| 612683.1_1_ | | Frame | Frame | | | | | | |
| 1:15527985 | | | | | | | | | |
| 9- | | | | | | | | | |
| 155279940: | | | | | | | | | |
| + | | | | | | | | | |
| T266625_4: | G061668 | lincRNA | lncRNA | — | — | — | — | — | — |
| 76104055- | | | | | | | | | |
| 76105674:- | | | | | | | | | |
| ENST00000 | GDPD5 | 5′ uORF | 5′ uORF | Detected | — | — | — | — | — |
| 336898.7_1_ | | | | | | | | | |
| 11:7523691 | | | | | | | | | |
| 3- | | | | | | | | | |
| 75236943:- | | | | | | | | | |
| ENST00000 | GNPTAB | Out-of- | Out-of- | — | — | — | — | — | — |
| 299314.11_1_ | | Frame | Frame | | | | | | |
| 12:102183 | | | | | | | | | |
| 791- | | | | | | | | | |
| 102224401:- | | | | | | | | | |
| ENST00000 | HMGN2P11 | Pseudogene | Pseudogene | — | — | — | — | — | — |
| 446054.1_1_ | | | | | | | | | |
| 7:84506012- | | | | | | | | | |
| 84506255:+ | | | | | | | | | |
| ENST00000 | IGF2R | 3′ dORF | 3′ dORF | — | — | — | — | — | — |
| 356956.5_2_ | | | | | | | | | |
| 6:16052742 | | | | | | | | | |
| 9- | | | | | | | | | |
| 160527663: | | | | | | | | | |
| + | | | | | | | | | |
| ENST00000 | IKBKAP | Out-of- | Out-of- | Detected | — | — | — | — | — |
| 374647.9_1_ | | Frame | Frame | | | | | | |
| 9:11168971 | | | | | | | | | |
| 4- | | | | | | | | | |
| 111692194:- | | | | | | | | | |
| ENST00000 | ITCH | 5′ uORF | 5′ uORF | Detected | — | — | — | — | — |
| 374864.8_3_ | | | | | | | | | |
| 20:3295720 | | | | | | | | | |
| 4- | | | | | | | | | |
| 32957252:+ | | | | | | | | | |
| ENST00000 | LAMP2 | Out-of- | Out-of- | — | — | — | — | — | — |
| 200639.8_2_ | | Frame | Frame | | | | | | |
| X:11958293 | | | | | | | | | |
| 8- | | | | | | | | | |
| 119589226:- | | | | | | | | | |
| ENST00000 | LARS | 5′ uORF | 5′ uORF | — | — | — | — | — | — |
| 394434.6_1_ | | | | | | | | | |
| 5:14556216 | | | | | | | | | |
| 7- | | | | | | | | | |
| 145562200:- | | | | | | | | | |
| ENST00000 | LGALS17A | Pseudogene | Pseudogene | Detected | — | — | — | — | — |
| 412609.5_1_ | | | | | | | | | |
| 19:4017005 | | | | | | | | | |
| 9- | | | | | | | | | |
| 40174276:+ | | | | | | | | | |
| ENST00000 | LSM14A | 5′ uORF | 5′ uORF | — | — | — | — | — | — |
| 589878.5_1_ | | | | | | | | | |
| 19:3466342 | | | | | | | | | |
| 9- | | | | | | | | | |
| 34663453:+ | | | | | | | | | |
| ENST00000 | MAP2K3 | 5′ uORF | 5′ uORF | Detected | — | — | — | — | — |
| 395491.6_1_ | | | | | | | | | |
| 17:2118813 | | | | | | | | | |
| 5- | | | | | | | | | |
| 21188198:+ | | | | | | | | | |
| ENST00000 | MIR646HG | lincRNA | lncRNA | Detected | — | — | — | — | — |
| 432910.5_1_ | | | | | | | | | |
| 20:5889587 | | | | | | | | | |
| 4- | | | | | | | | | |
| 58896255:+ | | | | | | | | | |
| ENST00000 | MLST8 | ncRNA | lncRNA | — | — | — | — | — | — |
| 568194.5_1_ | | Retained | | | | | | | |
| 16:2255935- | | Intron | | | | | | | |
| 2255977:+ | | | | | | | | | |
| ENST00000 | MMADHC | Out-of- | Out-of- | Detected | — | — | — | — | — |
| 428879.5_1_ | | Frame | Frame | | | | | | |
| 2:15043608 | | | | | | | | | |
| 1- | | | | | | | | | |
| 150438721:- | | | | | | | | | |
| ENST00000 | NBAS | Out-of- | Out-of- | — | — | — | — | — | — |
| 281513.9_1_ | | Frame | Frame | | | | | | |
| 2:15694206- | | | | | | | | | |
| 15694242:- | | | | | | | | | |
| ENST00000 | NIPA2 | Out-of- | Out-of- | Detected | — | — | — | — | — |
| 337451.7_1_ | | Frame | Frame | | | | | | |
| 15:2301447 | | | | | | | | | |
| 1- | | | | | | | | | |
| 23019844:- | | | | | | | | | |
| ENST00000 | NSUN5P2 | Pseudogene | Pseudogene | Detected | — | — | — | — | — |
| 485741.6_1_ | | | | | | | | | |
| 7:72424097- | | | | | | | | | |
| 72424166:- | | | | | | | | | |
| ENST00000 | PIGW | 5′ uORF | 5′ uORF | Detected | — | — | — | — | — |
| 614443.1_1_ | | | | | | | | | |
| 17:3489093 | | | | | | | | | |
| 8- | | | | | | | | | |
| 34891122:+ | | | | | | | | | |
| ENST00000 | PPP6R3 | Out-of- | Out-of- | — | — | — | — | — | — |
| 527403.6_2_ | | Frame | Frame | | | | | | |
| 11:6830522 | | | | | | | | | |
| 6- | | | | | | | | | |
| 68305268:+ | | | | | | | | | |
| ENST00000 | PRPF39 | ncRNA | lncRNA | Detected | — | — | — | — | — |
| 477626.5_1_ | | Retained | | | | | | | |
| 14:4556443 | | Intron | | | | | | | |
| 1- | | | | | | | | | |
| 45564512:+ | | | | | | | | | |
| ENST00000 | PRSS23 | Out-of- | Out-of- | — | — | — | — | — | — |
| 280258.5_1_ | | Frame | Frame | | | | | | |
| 11:8651886 | | | | | | | | | |
| 3- | | | | | | | | | |
| 86519148:+ | | | | | | | | | |
| ENST00000 | PTRH2 | 5′ uORF | 5′ uORF | — | — | — | — | — | — |
| 409433.2_1_ | | | | | | | | | |
| 17:5777749 | | | | | | | | | |
| 5- | | | | | | | | | |
| 57784778:- | | | | | | | | | |
| ENST00000 | RBPMS | 3′ dORF | 3′ dORF | — | — | — | — | — | — |
| 520191.5_1_ | | | | | | | | | |
| 8:30402888- | | | | | | | | | |
| 30402969:+ | | | | | | | | | |
| ENST00000 | RNF6 | 5′ uORF | 5′ uORF | Detected | — | — | — | — | — |
| 468480.5_1_ | | | | | | | | | |
| 13:2679538 | | | | | | | | | |
| 8- | | | | | | | | | |
| 26795430:- | | | | | | | | | |
| ENST00000 | RP1-39G22.7 | Antisense | lncRNA | Detected | — | — | — | — | — |
| 567508.1_1_ | | | | | | | | | |
| 1:40723236- | | | | | | | | | |
| 40723284:- | | | | | | | | | |
| ENST00000 | RP11-58C22.1 | 3′ dORF | 3′ dORF | Detected | — | — | — | — | — |
| 563764.2_2_ | | | | | | | | | |
| 16:7666941 | | | | | | | | | |
| 3- | | | | | | | | | |
| 76669506:+ | | | | | | | | | |
| ENST00000 | RP11-90H3.1 | Pseudogene | Pseudogene | Detected | — | — | — | — | — |
| 415279.1_1_ | | | | | | | | | |
| 1:10643493 | | | | | | | | | |
| 0- | | | | | | | | | |
| 106435587: | | | | | | | | | |
| + | | | | | | | | | |
| ENST00000 | RPS13 | ncRNA | lncRNA | — | — | — | — | — | — |
| 531908.5_1_ | | Reatined | | | | | | | |
| 11:1709712 | | Intron | | | | | | | |
| 2- | | | | | | | | | |
| 17097149:- | | | | | | | | | |
| ENST00000 | RPS14 | 5′ Overlap | 5′ Overlap | Detected | — | — | — | — | — |
| 312037.5_1_ | | uORF | uORF | | | | | | |
| 5:14982650 | | | | | | | | | |
| 6- | | | | | | | | | |
| 149829126:- | | | | | | | | | |
| ENST00000 | S100A11 | Out-of- | Out-of- | Detected | — | — | — | — | — |
| 271638.2_1_ | | Frame | Frame | | | | | | |
| 1:15200615 | | | | | | | | | |
| 8- | | | | | | | | | |
| 152006227:- | | | | | | | | | |
| ENST00000 | SEL1L | Out-of- | Out-of- | — | — | — | — | — | — |
| 336735.8_1_ | | Frame | Frame | | | | | | |
| 14:8197060 | | | | | | | | | |
| 2- | | | | | | | | | |
| 81972522:- | | | | | | | | | |
| ENST00000 | SESTD1 | Out-of- | Out-of- | Detected | — | — | — | — | — |
| 428443.7_1_ | | Frame | Frame | | | | | | |
| 2:18001117 | | | | | | | | | |
| 0- | | | | | | | | | |
| 180014060:- | | | | | | | | | |
| ENST00000 | SFT2D2 | 3′ dORF | 3′ dORF | Detected | — | — | — | — | — |
| 630869.1_1_ | | | | | | | | | |
| 1:16821209 | | | | | | | | | |
| 6- | | | | | | | | | |
| 168212303: | | | | | | | | | |
| + | | | | | | | | | |
| ENST00000 | SLC25A24P2 | Pseudogene | Pseudogene | — | — | — | — | — | — |
| 415059.1_1_ | | | | | | | | | |
| 1:10895275 | | | | | | | | | |
| 2- | | | | | | | | | |
| 108963771:- | | | | | | | | | |
| ENST00000 | SMOX | ncRNA | lncRNA | — | — | — | — | — | — |
| 484515.1_1_ | | Processed | | | | | | | |
| 20:4158069- | | Transcript | | | | | | | |
| 4162462:+ | | | | | | | | | |
| ENST00000 | SNAI2 | 5′ Overlap | 5′ Overlap | — | — | — | — | — | — |
| 020945.3_2_ | | uORF | uORF | | | | | | |
| 8:49832997- | | | | | | | | | |
| 49833910:- | | | | | | | | | |
| ENST00000 | SNX8 | Out-of- | Out-of- | Detected | — | — | — | — | — |
| 222990.7_1_ | | Frame | Frame | | | | | | |
| 7:2311567- | | | | | | | | | |
| 2317760:- | | | | | | | | | |
| ENST00000 | ST3GAL6-AS1 | Antisense | lncRNA | Detected | — | — | — | — | — |
| 488132.5_1_ | | | | | | | | | |
| 3:98434244- | | | | | | | | | |
| 98434552:- | | | | | | | | | |
| ENST00000 | TCP11L1 | 5′ uORF | 5′ uORF | — | — | — | — | — | — |
| 528107.5_2_ | | | | | | | | | |
| 11:3306171 | | | | | | | | | |
| 8- | | | | | | | | | |
| 33061820:+ | | | | | | | | | |
| ENST00000 | TENM4 | ncRNA | lncRNA | — | — | — | — | — | — |
| 528688.5_1_ | | Processed | | | | | | | |
| 11:7900853 | | Transcript | | | | | | | |
| 5- | | | | | | | | | |
| 79150030:- | | | | | | | | | |
| ENST00000 | TMBIM6 | Out-of- | Out-of- | Detected | — | — | — | — | — |
| 552370.5_1_ | | Frame | Frame | | | | | | |
| 12:5014678 | | | | | | | | | |
| 4- | | | | | | | | | |
| 50149448:+ | | | | | | | | | |
| ENST00000 | TMEM161B | 5′ Overlap | 5′ Overlap | Detected | — | — | — | — | — |
| 510089.5_1_ | | uORF | uORF | | | | | | |
| 5:87524264- | | | | | | | | | |
| 87564570:- | | | | | | | | | |
| ENST00000 | TMEM183A | 5′ Overlap | 5′ Overlap | Detected | — | — | — | — | — |
| 367242.3_1_ | | uORF | uORF | | | | | | |
| 1:20297653 | | | | | | | | | |
| 1- | | | | | | | | | |
| 202976618: | | | | | | | | | |
| + | | | | | | | | | |
| ENST00000 | TMF1 | 5′ uORF | 5′ uORF | Detected | — | — | — | — | — |
| 398559.6_1_ | | | | | | | | | |
| 3:69101351- | | | | | | | | | |
| 69101438:- | | | | | | | | | |
| ENST00000 | TNFRSF21 | 5′ uORF | 5′ uORF | — | — | — | — | — | — |
| 296861.2_1_ | | | | | | | | | |
| 6:47277434- | | | | | | | | | |
| 47277623:- | | | | | | | | | |
| ENST00000 | TNS3 | ncRNA | lncRNA | — | — | — | — | — | — |
| 469470.1_1_ | | Processed | | | | | | | |
| 7:47440019- | | Transcript | | | | | | | |
| 47440405:- | | | | | | | | | |
| ENST00000 | TPK1 | ncRNA | lncRNA | Detected | — | — | — | — | — |
| 548460.2_1_ | | Processed | | | | | | | |
| 7:14453251 | | Transcript | | | | | | | |
| 4- | | | | | | | | | |
| 144532616:- | | | | | | | | | |
| ENST00000 | TRIP6 | 5′ Overlap | 5′ Overlap | — | — | — | — | — | — |
| 417475.5_1_ | | uORF | uORF | | | | | | |
| 7:10046502 | | | | | | | | | |
| 5- | | | | | | | | | |
| 100465781: | | | | | | | | | |
| + | | | | | | | | | |
| ENST00000 | TWNK | ncRNA | lncRNA | Detected | — | — | — | — | — |
| 459764.1_1_ | | Processed | | | | | | | |
| 10:1027475 | | Transcript | | | | | | | |
| 80- | | | | | | | | | |
| 102749466: | | | | | | | | | |
| + | | | | | | | | | |
| ENST00000 | TXNRD1 | Out-of- | Out-of- | — | — | — | — | — | — |
| 526950.1_1_ | | Frame | Frame | | | | | | |
| 12:1047051 | | | | | | | | | |
| 13- | | | | | | | | | |
| 104707050: | | | | | | | | | |
| + | | | | | | | | | |
| ENST00000 | UBE2E3 | 5′ uORF | 5′ uORF | — | — | — | — | — | — |
| 410062.8_1_ | | | | | | | | | |
| 2:18184534 | | | | | | | | | |
| 3- | | | | | | | | | |
| 181846756: | | | | | | | | | |
| + | | | | | | | | | |
| ENST00000 | WAC-AS1 | Antisense | lncRNA | — | — | — | — | — | — |
| 528337.1_1_ | | | | | | | | | |
| 10:2881333 | | | | | | | | | |
| 7- | | | | | | | | | |
| 28813382:- | | | | | | | | | |
| ENST00000 | ZFP91 | Out-of- | Out-of- | — | — | — | — | — | — |
| 316059.6_1_ | | Frame | Frame | | | | | | |
| 11:5835234 | | | | | | | | | |
| 8- | | | | | | | | | |
| 58378460:+ | | | | | | | | | |
| ENST00000 | ZFR | Out-of- | Out-of- | Detected | — | — | — | — | — |
| 265069.12_1_ | | Frame | Frame | | | | | | |
| 5:3242018 | | | | | | | | | |
| 5- | | | | | | | | | |
| 32444730:- | | | | | | | | | |
| ENST00000 | ZFYVE26 | 5′ uORF | 5′ uORF | — | — | — | — | — | — |
| 347230.8_2_ | | | | | | | | | |
| 14:6828272 | | | | | | | | | |
| 2- | | | | | | | | | |
| 68283293:- | | | | | | | | | |
| ENST00000 | ZMAT2 | Out-of- | Out-of- | Detected | — | — | — | — | — |
| 274712.7_1_ | | Frame | Frame | | | | | | |
| 5:14008044 | | | | | | | | | |
| 0- | | | | | | | | | |
| 140083534: | | | | | | | | | |
| + | | | | | | | | | |
| ENST00000 | ZNF32 | 5′ Overlap | 5′ Overlap | — | — | — | — | — | — |
| 395797.1_1_ | | uORF | uORF | | | | | | |
| 10:4414023 | | | | | | | | | |
| 9- | | | | | | | | | |
| 44144057:- | | | | | | | | | |
| ENST00000 | ZNF638 | 5′ uORF | 5′ uORF | — | — | — | — | — | — |
| 410075.5_1_ | | | | | | | | | |
| 2:71576016- | | | | | | | | | |
| 71576058:+ | | | | | | | | | |
| TCONS_00 | linc-KIN-6 | lincRNA | lncRNA | — | — | — | — | — | — |
| 018434_10: | | | | | | | | | |
| 10826571- | | | | | | | | | |
| 10834395:- | | | | | | | | | |
| ENST00000 | PCBP1 | Out-of- | Out-of- | — | Detected | — | — | — | — |
| 303577.6_1_ | | Frame | Frame | | | | | | |
| 2:70315071- | | | | | | | | | |
| 70315098:+ | | | | | | | | | |
| ENST00000 | RASGRP3 | 5′ uORF | 5′ uORF | Detected | Detected | — | — | — | — |
| 402538.7_1_ | | | | | | | | | |
| 2:33740127- | | | | | | | | | |
| 33740184:+ | | | | | | | | | |
| ENST00000 | BTN3A2 | 5′ Overlap | 5′ Overlap | Detected | — | — | Detected | — | — |
| 532865.5_2_ | | uORF | uORF | | | | | | |
| 6:26365529- | | | | | | | | | |
| 26368244:+ | | | | | | | | | |
| ENST00000 | C6orf62 | 5′ uORF | 5′ uORF | Detected | — | Detected | Detected | — | — |
| 378119.8_1_ | | | | | | | | | |
| 6:24720562- | | | | | | | | | |
| 24720727:- | | | | | | | | | |
| ENST00000 | MBOAT7 | Out-of- | Out-of- | Detected | — | — | — | Detected | — |
| 245615.5_1_ | | Frame | Frame | | | | | | |
| 19:5469229 | | | | | | | | | |
| 4- | | | | | | | | | |
| 54692348:- | | | | | | | | | |
| ENST00000 | EIF4G2 | Out-of- | Out-of- | Detected | — | — | — | — | Detected |
| 339995.9_2_ | | Frame | Frame | | | | | | |
| 11:1082506 | | | | | | | | | |
| 7- | | | | | | | | | |
| 10825719:- | | | | | | | | | |
| ENST00000 | CLIP1 | 5′ uORF | 5′ uORF | — | — | — | — | — | — |
| 539080.1_1_ | | | | | | | | | |
| 12:1228650 | | | | | | | | | |
| 79- | | | | | | | | | |
| 122884682:- | | | | | | | | | |
| ENST00000 | CUL5 | 5′ uORF | 5′ uORF | Detected | — | — | — | — | — |
| 393094.6_1_ | | | | | | | | | |
| 11:1078796 | | | | | | | | | |
| 97- | | | | | | | | | |
| 107879871: | | | | | | | | | |
| + | | | | | | | | | |
| ENST00000 | ZMYND8 | 5′ uORF | 5′ uORF | Detected | — | — | — | — | — |
| 446994.6_1_ | | | | | | | | | |
| 20:4598552 | | | | | | | | | |
| 7- | | | | | | | | | |
| 45985632:- | | | | | | | | | |
| ENST00000 | ABL1 | Out-of- | Out-of- | Detected | — | — | — | — | — |
| 372348.6_1_ | | Frame | Frame | | | | | | |
| 9:13372953 | | | | | | | | | |
| 7- | | | | | | | | | |
| 133729609: | | | | | | | | | |
| + | | | | | | | | | |
| ENST00000 | ADAR | 5′ Overlap | 5′ Overlap | — | — | — | — | — | — |
| 368474.8_1_ | | uORF | uORF | | | | | | |
| 1:15457505 | | | | | | | | | |
| 8- | | | | | | | | | |
| 154580648:- | | | | | | | | | |
| ENST00000 | ADGRF5 | 5′ uORF | 5′ uORF | — | — | — | — | — | — |
| 283296.11_1_ | | | | | | | | | |
| 6:4688956 | | | | | | | | | |
| 1- | | | | | | | | | |
| 46889594:- | | | | | | | | | |
| ENST00000 | ATP2B1 | 5′ uORF | 5′ uORF | — | — | — | — | — | — |
| 551310.1_1_ | | | | | | | | | |
| 12:9004980 | | | | | | | | | |
| 5- | | | | | | | | | |
| 90103048:- | | | | | | | | | |
| ENST00000 | BOLA3-AS1 | Antisense | lncRNA | Detected | — | — | — | — | — |
| 529783.1_1_ | | | | | | | | | |
| 2:74375310- | | | | | | | | | |
| 74375499:+ | | | | | | | | | |
| ENST00000 | BPGM | 5′ uORF | 5′ uORF | — | — | — | — | — | — |
| 418040.5_1_ | | | | | | | | | |
| 7:13433159 | | | | | | | | | |
| 0- | | | | | | | | | |
| 134346220: | | | | | | | | | |
| + | | | | | | | | | |
| ENST00000 | C6orf48 | 5′ Overlap | 5′ Overlap | — | — | — | — | — | — |
| 395789.5_1_ | | uORF | uORF | | | | | | |
| 6:31802780- | | | | | | | | | |
| 31805136:+ | | | | | | | | | |
| ENST00000 | CALCOCO2 | 5′ Overlap | 5′ Overlap | — | — | — | — | — | — |
| 511697.1_1_ | | uORF | uORF | | | | | | |
| 17:4691906 | | | | | | | | | |
| 9- | | | | | | | | | |
| 46919841:+ | | | | | | | | | |
| ENST00000 | CHI3L2 | 5′ Overlap | 5′ Overlap | — | — | — | — | — | — |
| 466741.5_1_ | | uORF | uORF | | | | | | |
| 1:11177235 | | | | | | | | | |
| 9- | | | | | | | | | |
| 111772476: | | | | | | | | | |
| + | | | | | | | | | |
| ENST00000 | CKMT2-AS1 | Antisense | lncRNA | — | — | — | — | — | — |
| 512287.1_1_ | | | | | | | | | |
| 5:80531836- | | | | | | | | | |
| 80531875:- | | | | | | | | | |
| ENST00000 | CREB3 | 5′ Overlap | 5′ Overlap | Detected | — | — | — | — | — |
| 353704.2_2_ | | uORF | uORF | | | | | | |
| 9:35732632- | | | | | | | | | |
| 35732818:+ | | | | | | | | | |
| ENST00000 | CTD-2006C1.2 | Antisense | lncRNA | Detected | — | — | — | — | — |
| 406892.6_1_ | | | | | | | | | |
| 19:1209863 | | | | | | | | | |
| 5- | | | | | | | | | |
| 12112633:+ | | | | | | | | | |
| ENST00000 | FAM102A | Out-of- | Out-of- | — | — | — | — | — | — |
| 373095.5_1_ | | Frame | Frame | | | | | | |
| 9:13071044 | | | | | | | | | |
| 5- | | | | | | | | | |
| 130712742:- | | | | | | | | | |
| ENST00000 | FAM65A | 5′ Overlap | 5′ Overlap | — | — | — | — | — | — |
| 566920.5_3_ | | uORF | uORF | | | | | | |
| 16:6756396 | | | | | | | | | |
| 7- | | | | | | | | | |
| 67572402:+ | | | | | | | | | |
| ENST00000 | GOLGA1 | 5′ uORF | 5′ uORF | Detected | — | — | — | — | — |
| 373555.8_1_ | | | | | | | | | |
| 9:12770107 | | | | | | | | | |
| 3- | | | | | | | | | |
| 127701109:- | | | | | | | | | |
| ENST00000 | GOLGA3 | 5′ uORF | 5′ uORF | Detected | — | — | — | — | — |
| 545875.4_1_ | | | | | | | | | |
| 12:1333988 | | | | | | | | | |
| 59- | | | | | | | | | |
| 133405404:- | | | | | | | | | |
| ENST00000 | IPO7 | Out-of- | Out-of- | — | — | — | — | — | — |
| 527431.1_1_ | | Frame | Frame | | | | | | |
| 11:9406310- | | | | | | | | | |
| 9430044:+ | | | | | | | | | |
| ENST00000 | LINC01133 | lincRNA | lncRNA | — | — | — | — | — | — |
| 443364.6_1_ | | | | | | | | | |
| 1:15993117 | | | | | | | | | |
| 0- | | | | | | | | | |
| 159935305: | | | | | | | | | |
| + | | | | | | | | | |
| ENST00000 | LNX1 | Out-of- | Out-of- | — | — | — | — | — | — |
| 306888.6_1_ | | Frame | Frame | | | | | | |
| 4:54364989- | | | | | | | | | |
| 54373573:- | | | | | | | | | |
| ENST00000 | MTIF2 | ncRNA | lncRNA | Detected | — | — | — | — | — |
| 446660.5_1_ | | Processed | | | | | | | |
| 2:55490938- | | Transcript | | | | | | | |
| 55494722:- | | | | | | | | | |
| ENST00000 | NAA50 | 5′ Overlap | 5′ Overlap | Detected | — | — | — | — | — |
| 616174.1_1_ | | uORF | uORF | | | | | | |
| 3:11344293 | | | | | | | | | |
| 9- | | | | | | | | | |
| 113464857:- | | | | | | | | | |
| ENST00000 | NBPF3 | ncRNA | lncRNA | — | — | — | — | — | — |
| 478653.6_1_ | | Processed | | | | | | | |
| 1:21780423- | | Transcript | | | | | | | |
| 21780552:+ | | | | | | | | | |
| ENST00000 | NME1 | 3′ dORF | 3′ dORF | Detected | — | — | — | — | — |
| 393196.7_1_ | | | | | | | | | |
| 17:4923925 | | | | | | | | | |
| 4- | | | | | | | | | |
| 49239476:+ | | | | | | | | | |
| ENST00000 | NXPE3 | 5′ uORF | 5′ uORF | — | — | — | — | — | — |
| 495842.5_1_ | | | | | | | | | |
| 3:10150426 | | | | | | | | | |
| 2- | | | | | | | | | |
| 101504385: | | | | | | | | | |
| + | | | | | | | | | |
| ENST00000 | PDCL | Out-of- | Out-of- | — | — | — | — | — | — |
| 259467.8_1_ | | Frame | Frame | | | | | | |
| 9:12558546 | | | | | | | | | |
| 1- | | | | | | | | | |
| 125589017:- | | | | | | | | | |
| ENST00000 | PJA2 | 5′ Overlap | 5′ Overlap | Detected | — | — | — | — | — |
| 361189.6_1_ | | uORF | uORF | | | | | | |
| 5:10871728 | | | | | | | | | |
| 7- | | | | | | | | | |
| 108719181:- | | | | | | | | | |
| ENST00000 | POLB | 5′ Overlap | 5′ Overlap | Detected | — | — | — | — | — |
| 532157.5_1_ | | | | | | | | | |
| 8:42196130- | | | | | | | | | |
| 42202487:+ | | | | | | | | | |
| ENST00000 | POMP | Out-of- | Out-of- | Detected | — | — | — | — | — |
| 380842.4_1_ | | Frame | Frame | | | | | | |
| 13:2923659 | | | | | | | | | |
| 2- | | | | | | | | | |
| 29238659:+ | | | | | | | | | |
| ENST00000 | PRR4 | ncRNA | lncRNA | — | — | — | — | — | — |
| 546265.1_1_ | | Processed | | | | | | | |
| 12:1127374 | | Transcript | | | | | | | |
| 1- | | | | | | | | | |
| 11324198:- | | | | | | | | | |
| ENST00000 | RP11- | lincRNA | lncRNA | — | — | — | — | — | — |
| 433510.2_1_ | 357H14.17 | | | | | | | | |
| 17:4671350 | | | | | | | | | |
| 3- | | | | | | | | | |
| 46713704:- | | | | | | | | | |
| ENST00000 | RPL23AP82 | Pseudogene | Pseudogene | — | — | — | — | — | — |
| 496652.5_1_ | | | | | | | | | |
| 22:5122171 | | | | | | | | | |
| 8- | | | | | | | | | |
| 51227271:+ | | | | | | | | | |
| ENST00000 | RPL7L1P8 | Pseudogene | Pseudogene | — | — | — | — | — | — |
| 469911.1_1_ | | | | | | | | | |
| 3:18232869 | | | | | | | | | |
| 4- | | | | | | | | | |
| 182329198: | | | | | | | | | |
| + | | | | | | | | | |
| ENST00000 | RPS27 | ncRNA | lncRNA | Detected | — | — | — | — | — |
| 493224.5_1_ | | Processed | | | | | | | |
| 1:15396412 | | Transcript | | | | | | | |
| 6- | | | | | | | | | |
| 153964580: | | | | | | | | | |
| + | | | | | | | | | |
| ENST00000 | RRS1 | 5′ uORF | 5′ uORF | Detected | — | — | — | — | — |
| 320270.3_1_ | | | | | | | | | |
| 8:67341274- | | | | | | | | | |
| 67341313:+ | | | | | | | | | |
| ENST00000 | SEC61B | 5′ Overlap | 5′ Overlap | — | — | — | — | — | — |
| 223641.4_1_ | | uORF | uORF | | | | | | |
| 9:10198462 | | | | | | | | | |
| 5- | | | | | | | | | |
| 101992644: | | | | | | | | | |
| + | | | | | | | | | |
| ENST00000 | SLC39A9 | 5′ uORF | 5′ uORF | Detected | — | — | — | — | — |
| 556605.5_1_ | | | | | | | | | |
| 14:6986541 | | | | | | | | | |
| 4- | | | | | | | | | |
| 69865501:+ | | | | | | | | | |
| ENST00000 | SNHG7 | Antisense | lncRNA | Detected | — | — | — | — | — |
| 414282.5_1_ | | | | | | | | | |
| 9:13962151 | | | | | | | | | |
| 9- | | | | | | | | | |
| 139622634:- | | | | | | | | | |
| ENST00000 | TGIF1 | 5′ uORF | 5′ uORF | Detected | — | — | — | — | — |
| 407501.6_1_ | | | | | | | | | |
| 18:3449895- | | | | | | | | | |
| 3450350:+ | | | | | | | | | |
| ENST00000 | TIMP1 | Out-of- | Out-of- | — | — | — | — | — | — |
| 218388.8_1_ | | Frame | Frame | | | | | | |
| X:47445059 | | | | | | | | | |
| -47446002:+ | | | | | | | | | |
| ENST00000 | TM2D1 | 3′ dORF | 3′ dORF | — | — | — | — | — | — |
| 488206.5_1_ | | | | | | | | | |
| 1:62175075- | | | | | | | | | |
| 62189407:- | | | | | | | | | |
| ENST00000 | TMBIM1 | 5′ uORF | 5′ uORF | — | — | — | — | — | — |
| 453776.5_1_ | | | | | | | | | |
| 2:21915056 | | | | | | | | | |
| 1- | | | | | | | | | |
| 219150675:- | | | | | | | | | |
| ENST00000 | TMEM87A | 5′ uORF | 5′ uORF | — | — | — | — | — | — |
| 448392.5_1_ | | | | | | | | | |
| 15:4256560 | | | | | | | | | |
| 1- | | | | | | | | | |
| 42565628:- | | | | | | | | | |
| ENST00000 | TRMT6 | 5′ Overlap | 5′ Overlap | — | — | — | — | — | — |
| 473131.5_1_ | | uORF | uORF | | | | | | |
| 20:5924535- | | | | | | | | | |
| 5931132:- | | | | | | | | | |
| ENST00000 | USP16 | 5′ uORF | 5′ uORF | | — | — | — | — | — |
| 399976.6_1_ | | | | | | | | | |
| 21:3039707 | | | | | | | | | |
| 1- | | | | | | | | | |
| 30400211:+ | | | | | | | | | |
| ENST00000 | ZC3H11A | 5′ uORF | 5′ uORF | — | — | — | — | — | — |
| 638800.1_1_ | | | | | | | | | |
| 1:20376562 | | | | | | | | | |
| 4- | | | | | | | | | |
| 203770741: | | | | | | | | | |
| + | | | | | | | | | |
| ENST00000 | ZNF213-AS1 | Antisense | lncRNA | — | — | — | — | — | — |
| 572691.2_2_ | | | | | | | | | |
| 16:3179460- | | | | | | | | | |
| 3179646:- | | | | | | | | | |
| TCONS_00 | linc-FDX1 | lincRNA | lncRNA | — | — | — | — | — | — |
| 019109_11: | | | | | | | | | |
| 110207337- | | | | | | | | | |
| 110252699: | | | | | | | | | |
| + | | | | | | | | | |
| T267797_4: | nan | lincRNA | lncRNA | — | — | — | — | — | — |
| 88400064- | | | | | | | | | |
| 88403668:+ | | | | | | | | | |
| T350279_8: | nan | lincRNA | lncRNA | — | — | — | — | — | — |
| 120845279- | | | | | | | | | |
| 120865310: | | | | | | | | | |
| + | | | | | | | | | |
| ENST00000 | BCLAF1 | 5′ uORF | 5′ uORF | Detected | Detected | — | — | — | — |
| 640069.1_1_ | | | | | | | | | |
| 6:13661088 | | | | | | | | | |
| 2- | | | | | | | | | |
| 136610939:- | | | | | | | | | |
| ENST00000 | NRAS | 3′ dORF | 3′ dORF | Detected | Detected | — | — | — | — |
| 369535.4_1_ | | | | | | | | | |
| 1:11525015 | | | | | | | | | |
| 5- | | | | | | | | | |
| 115250203:- | | | | | | | | | |
| ENST00000 | SLC39A9 | Out-of- | Out-of- | Detected | Detected | — | — | — | — |
| 557046.1_1_ | | Frame | Frame | | | | | | |
| 14:6990896 | | | | | | | | | |
| 5- | | | | | | | | | |
| 69921585:+ | | | | | | | | | |
| ENST00000 | VPS29 | 5′ Overlap | 5′ Overlap | Detected | Detected | — | — | — | — |
| 447578.6_1_ | | uORF | uORF | | | | | | |
| 12:1109309 | | | | | | | | | |
| 33- | | | | | | | | | |
| 110930975:- | | | | | | | | | |
| ENST00000 | ADCY9 | 5′ uORF | 5′ uORF | — | — | Detected | — | — | — |
| 294016.7_1_ | | | | | | | | | |
| 16:4165851- | | | | | | | | | |
| 4165998:- | | | | | | | | | |
| ENST00000 | PRRC2A | 5′ Overlap | 5′ Overlap | Detected | — | Detected | — | — | — |
| 376007.8_1_ | | uORF | uORF | | | | | | |
| 6:31588511- | | | | | | | | | |
| 31590634:+ | | | | | | | | | |
| ENST00000 | TCTN2 | 5′ uORF | 5′ uORF | — | — | Detected | — | — | — |
| 303372.6_1_ | | | | | | | | | |
| 12:1241556 | | | | | | | | | |
| 84- | | | | | | | | | |
| 124155726: | | | | | | | | | |
| + | | | | | | | | | |
| ENST00000 | TMEM60 | 5′ Overlap | 5′ Overlap | Detected | — | Detected | — | — | — |
| 257663.3_1_ | | uORF | uORF | | | | | | |
| 7:77423607- | | | | | | | | | |
| 77427674:- | | | | | | | | | |
| ENST00000 | RPRD1A | Out-of- | Out-of- | Detected | — | — | — | — | — |
| 399022.8_1_ | | Frame | Frame | | | | | | |
| 18:3361369 | | | | | | | | | |
| 5- | | | | | | | | | |
| 33613761:- | | | | | | | | | |
| T301592_6: | nan | lincRNA | lncRNA | — | — | — | — | — | — |
| 32441186- | | | | | | | | | |
| 32441927:+ | | | | | | | | | |
| ENST00000 | PCDH7 | 5′ uORF | 5′ uORF | — | — | — | — | — | Detected |
| 361762.3_2_ | | | | | | | | | |
| 4:30722246- | | | | | | | | | |
| 30722306:+ | | | | | | | | | |
| ENST00000 | ELOVL1 | 5′ Overlap | 5′ Overlap | — | — | — | — | — | — |
| 487209.5_1_ | | uORF | uORF | | | | | | |
| 1:43830638- | | | | | | | | | |
| 43833598:- | | | | | | | | | |
| ENST00000 | NUDCD2 | ncRNA | lncRNA | Detected | — | Detected | — | — | — |
| 521797.l_l_ | | Processed | | | | | | | |
| 5:16288400 | | Transcript | | | | | | | |
| 4- | | | | | | | | | |
| 162884578:- | | | | | | | | | |
| ENST00000 | RP11-783K16.5 | Antisense | lncRNA | Detected | — | — | — | — | Detected |
| 544553.1_1_ | | | | | | | | | |
| 11:6401345 | | | | | | | | | |
| 3- | | | | | | | | | |
| 64013543:+ | | | | | | | | | |
| ENST00000 | DDX3X | 3′ dORF | 3′ dORF | — | — | Detected | — | — | — |
| 441189.3_1_ | | | | | | | | | |
| X:41200742- | | | | | | | | | |
| 41206988:+ | | | | | | | | | |
| ENST00000 | MGAT4B | 5′ Overlap | 5′ Overlap | Detected | — | — | — | — | Detected |
| 292591.11_1_ | | uORF | uORF | | | | | | |
| 5:1792289 | | | | | | | | | |
| 08- | | | | | | | | | |
| 179233794:- | | | | | | | | | |
| ENST00000 | ATL2 | 3′Overlap | 3′Overlap | Detected | — | — | — | — | — |
| 477642.5_1_ | | dORF | dORF | | | | | | |
| 2:38525261- | | | | | | | | | |
| 38527422:- | | | | | | | | | |
| ENST00000 | C19orf48 | 5′ uORF | 5′ uORF | Detected | — | — | — | — | — |
| 345523.8_1_ | | | | | | | | | |
| 19:5130258 | | | | | | | | | |
| 4- | | | | | | | | | |
| 51305485:- | | | | | | | | | |
| ENST00000 | CHIC1 | 3′ dORF | 3′ dORF | — | — | — | — | — | — |
| 373504.10_1_ | | | | | | | | | |
| X:7290088 | | | | | | | | | |
| 8- | | | | | | | | | |
| 72901020:+ | | | | | | | | | |
| ENST00000 | CTD- | lincRNA | lncRNA | — | — | — | — | — | — |
| 606994.1_1_ | 2186M15.3 | | | | | | | | |
| 5:32174612- | | | | | | | | | |
| 32175047:+ | | | | | | | | | |
| ENST00000 | DARS2 | 5′ Overlap | 5′ Overlap | — | — | — | — | — | — |
| 361951.4_1__ | | uORF | uORF | | | | | | |
| 1:17379435 | | | | | | | | | |
| 1- | | | | | | | | | |
| 173795834: | | | | | | | | | |
| + | | | | | | | | | |
| ENST00000 | EIF3E | 5′ Overlap | 5′ Overlap | Detected | — | — | — | — | — |
| 518345.1_1_ | | uORF | uORF | | | | | | |
| 8:10925407 | | | | | | | | | |
| 8- | | | | | | | | | |
| 109254135:- | | | | | | | | | |
| ENST00000 | FAM3B | Out-of | Out-of- | — | — | — | — | — | — |
| 479810.6_1_ | | Frame | Frame | | | | | | |
| 21:4269493 | | | | | | | | | |
| 0- | | | | | | | | | |
| 42716440:+ | | | | | | | | | |
| ENST00000 | FHL2 | 5′ Overlap | 5′ Overlap | — | — | — | — | — | — |
| 409807.5_1_ | | uORF | uORF | | | | | | |
| 2:10601311 | | | | | | | | | |
| 3- | | | | | | | | | |
| 106013155:- | | | | | | | | | |
| ENST00000 | GMPS | Out-of | Out-of- | — | — | — | — | — | — |
| 496455.6_1_ | | Frame | Frame | | | | | | |
| 3:15561148 | | | | | | | | | |
| 7- | | | | | | | | | |
| 155621652: | | | | | | | | | |
| + | | | | | | | | | |
| ENST00000 | KAT6A | 5′ uORF | 5′ uORF | Detected | — | — | — | — | — |
| 265713.6_2_ | | | | | | | | | |
| 8:41906525- | | | | | | | | | |
| 41906624:- | | | | | | | | | |
| ENST00000 | KAT6B | 5′ uORF | 5′ uORF | Detected | — | — | — | — | — |
| 372724.5_1_ | | | | | | | | | |
| 10:7659850 | | | | | | | | | |
| 7- | | | | | | | | | |
| 76602417:+ | | | | | | | | | |
| ENST00000 | KAT6B | Out-of | Out-of- | Detected | — | — | — | — | — |
| 372725.5_1_ | | Frame | Frame | | | | | | |
| 10:7660269 | | | | | | | | | |
| 7- | | | | | | | | | |
| 76602946:+ | | | | | | | | | |
| ENST00000 | KIF23 | Out-of | Out-of- | — | — | — | — | — | — |
| 395392.6_2_ | | Frame | Frame | | | | | | |
| 15:6970978 | | | | | | | | | |
| 5- | | | | | | | | | |
| 69714354:+ | | | | | | | | | |
| ENST00000 | MED17 | 5′ uORF | 5′ uORF | Detected | — | — | — | — | — |
| 640804.1_1_ | | | | | | | | | |
| 11:9351753 | | | | | | | | | |
| 0- | | | | | | | | | |
| 93517563:+ | | | | | | | | | |
| ENST00000 | MED20 | 5′ Overlap | 5′ Overlap | Detected | — | — | — | — | — |
| 482361.1_1_ | | uORF | uORF | | | | | | |
| 6:41877193- | | | | | | | | | |
| 41888846:- | | | | | | | | | |
| ENST00000 | MT1F | 3′Overlap | 3′Overlap | — | — | — | — | — | — |
| 334350.6_1_ | | dORF | dORF | | | | | | |
| 16:5669163 | | | | | | | | | |
| 0- | | | | | | | | | |
| 56693012:+ | | | | | | | | | |
| ENST00000 | PARM1 | Out-of | Out-of- | — | — | — | — | — | — |
| 307428.7_1_ | | Frame | Frame | | | | | | |
| 4:75858563- | | | | | | | | | |
| 75937719:+ | | | | | | | | | |
| ENST00000 | PTGES3 | 3′ dORF | 3′ dORF | Detected | — | — | — | — | — |
| 537473.2_1_ | | | | | | | | | |
| 12:5705810 | | | | | | | | | |
| 3- | | | | | | | | | |
| 57058217:- | | | | | | | | | |
| ENST00000 | RP11-314C16.1 | lincRNA | lncRNA | — | — | — | — | — | — |
| 429060.1_1_ | | | | | | | | | |
| 6:8784460- | | | | | | | | | |
| 8785356:+ | | | | | | | | | |
| ENST00000 | TBL1XR1 | Out-of | Out-of- | — | — | — | — | — | — |
| 430069.5_1_ | | Frame | Frame | | | | | | |
| 3:17677167 | | | | | | | | | |
| 6- | | | | | | | | | |
| 176782752:- | | | | | | | | | |
| ENST00000 | UBE2E3 | 5′ Overlap | 5′ Overlap | Detected | — | — | — | — | — |
| 410062.8_1_ | | uORF | uORF | | | | | | |
| 2:18184675 | | | | | | | | | |
| 5- | | | | | | | | | |
| 181848786: | | | | | | | | | |
| + | | | | | | | | | |
| ENST00000 | ZNF174 | 5′ uORF | 5′ uORF | — | — | — | — | — | — |
| 344823.9_1_ | | | | | | | | | |
| 16:3451289- | | | | | | | | | |
| 3451709:+ | | | | | | | | | |
| ENST00000 | ZNF503-AS2 | Antisense | lncRNA | — | — | — | — | — | — |
| 466942.2_1_ | | | | | | | | | |
| 10:7716730 | | | | | | | | | |
| 5- | | | | | | | | | |
| 77167401:+ | | | | | | | | | |
| ENST00000 | C7orf73 | 5′ Overlap | 5′ Overlap | Detected | — | — | — | — | Detected |
| 507606.2_1_ | | uORF | uORF | | | | | | |
| 7:13534728 | | | | | | | | | |
| 3- | | | | | | | | | |
| 135358947: | | | | | | | | | |
| + | | | | | | | | | |
| ENST00000 | GEMIN6 | 5′ uORF | 5′ uORF | Detected | — | — | — | — | Detected |
| 409011.5_2_ | | | | | | | | | |
| 2:39005336- | | | | | | | | | |
| 39005417:+ | | | | | | | | | |
| ENST00000 | KIAA0355 | 5′ uORF | 5′ uORF | — | — | — | — | — | Detected |
| 299505.6_1_ | | | | | | | | | |
| 19:3479111 | | | | | | | | | |
| 8- | | | | | | | | | |
| 34791184:+ | | | | | | | | | |
| ENST00000 | KRT18P62 | Pseudogene | Pseudogene | Detected | — | — | — | — | Detected |
| 446618.1_1_ | | | | | | | | | |
| 22:1924587 | | | | | | | | | |
| 9- | | | | | | | | | |
| 19246326:+ | | | | | | | | | |
| ENST00000 | MLLT3 | 5′ uORF | 5′ uORF | Detected | — | — | — | — | Detected |
| 380338.8_1_ | | | | | | | | | |
| 9:20622330- | | | | | | | | | |
| 20622507:- | | | | | | | | | |
| ENST00000 | NOL11 | Out-of- | Out-of- | — | — | — | — | — | Detected |
| 253247.8_1_ | | Frame | Frame | | | | | | |
| 17:6573210 | | | | | | | | | |
| 3- | | | | | | | | | |
| 65732808:+ | | | | | | | | | |
| ENST00000 | RP11-381K20.2 | Antisense | lncRNA | Detected — | — | — | — | Detected | — |
| 514616.5_1_ | | | | | | | | | |
| 5:13722385 | | | | | | | | | |
| 5- | | | | | | | | | |
| 137224842:- | | | | | | | | | |
| ENST00000 | RPL13AP23 | Pseudogene | Pseudogene | — | — | — | — | — | Detected |
| 480739.2_1_ | | | | | | | | | |
| 12:5806887 | | | | | | | | | |
| 7- | | | | | | | | | |
| 58069030:+ | | | | | | | | | |
| ENST00000 | USP53 | ncRNA | lncRNA | — | — | — | — | — | Detected |
| 510737.1_1_ | | Processed | | | | | | | |
| 4:12016039 | | Transcript | | | | | | | |
| 5- | | | | | | | | | |
| 120160458: | | | | | | | | | |
| + | | | | | | | | | |
| ENST00000 | ZFAND6 | 3′ dORF | 3′ dORF | Detected | — | — | — | — | Detected |
| 613266.4_1_ | | | | | | | | | |
| 15:8043021 | | | | | | | | | |
| 2- | | | | | | | | | |
| 80430356:+ | | | | | | | | | |
| ENST00000 | USP34 | Out-of- | Out-of- | Detected | — | — | Detected | — | Detected |
| 398571.6_2_ | | Frame | Frame | | | | | | |
| 2:61633207- | | | | | | | | | |
| 61647956:- | | | | | | | | | |
| ENST00000 | PKIG | 5′ uORF | 5′ uORF | — | — | — | — | — | — |
| 372894.7_1_ | | | | | | | | | |
| 20:4316052 | | | | | | | | | |
| 1- | | | | | | | | | |
| 43218474:+ | | | | | | | | | |
| ENST00000 | SNRK | 5′ uORF | 5′ uORF | Detected | — | — | — | — | — |
| 429705.6_1_ | | | | | | | | | |
| 3:43328077- | | | | | | | | | |
| 43344643:+ | | | | | | | | | |
| ENST00000 | AZIN1 | Out-of- | Out-of- | Detected | Detected | — | — | — | — |
| 347770.8_1_ | | Frame | Frame | | | | | | |
| 8:10385200 | | | | | | | | | |
| 5- | | | | | | | | | |
| 103852041:- | | | | | | | | | |
| ENST00000 | WAPL | Out-of- | Out-of- | Detected | Detected | — | — | — | — |
| 298767.9_1_ | | Frame | Frame | | | | | | |
| 10:8827755 | | | | | | | | | |
| 8- | | | | | | | | | |
| 88277621:- | | | | | | | | | |
| ENST00000 | TMEM117 | Out-of- | Out-of- | Detected | Detected | — | — | — | Detected |
| 546387.1_1_ | | Frame | Frame | | | | | | |
| 12:4423858 | | | | | | | | | |
| 1- | | | | | | | | | |
| 44238653+ | | | | | | | | | |
| ENST00000 | MTND2P28 | Pseudogene | Pseudogene | Detected | — | — | — | — | Detected |
| 457540.1_1_ | | | | | | | | | |
| 1:565028- | | | | | | | | | |
| 565094:+ | | | | | | | | | |
| ENST00000 | SRSF9 | Out-of- | Out-of- | Detected | Detected | — | — | — | Detected |
| 229390.7_1_ | | Frame | Frame | | | | | | |
| 12:1209018 | | | | | | | | | |
| 92- | | | | | | | | | |
| 120907330:- | | | | | | | | | |
| ENST00000 | MORF4L1 | 5′ uORF | 5′ uORF | — | — | — | — | — | — |
| 559345.5_1_ | | | | | | | | | |
| 15:7916554 | | | | | | | | | |
| 8- | | | | | | | | | |
| 79172860:+ | | | | | | | | | |
| ENST00000 | ASL | 5′ Overlap | 5′ Overlap | Detected | — | — | — | — | — |
| 362000.9_1_ | | uORF | uORF | | | | | | |
| 7:65540928- | | | | | | | | | |
| 65547388:+ | | | | | | | | | |
| ENST00000 | KCNN4 | 5′ Overlap | 5′ Overlap | — | — | — | — | — | — |
| 615047.4_1_ | | uORF | uORF | | | | | | |
| 19:4427842 | | | | | | | | | |
| 8- | | | | | | | | | |
| 44285151:- | | | | | | | | | |
| ENST00000 | MRPS18B | Out-of- | Out-of- | Detected | — | — | — | — | — |
| 259873.4_1_ | | Frame | Frame | | | | | | |
| 6:30587521- | | | | | | | | | |
| 30590615:+ | | | | | | | | | |
| ENST00000 | MTIF3 | 5′ Overlap | 5′ Overlap | — | — | — | — | — | — |
| 493719.5_1_ | | uORF | uORF | | | | | | |
| 13:2801456 | | | | | | | | | |
| 9- | | | | | | | | | |
| 28024213:- | | | | | | | | | |
| ENST00000 | NR3C1 | 5′ uORF | 5′ uORF | Detected | — | — | — | — | — |
| 394464.6_1_ | | | | | | | | | |
| 5:14278041 | | | | | | | | | |
| 6- | | | | | | | | | |
| 142782924:- | | | | | | | | | |
| ENST00000 | TESMIN | 3′Overlap | 3′Overlap | — | — | — | — | — | — |
| 438124.2_1_ | | dORF | dORF | | | | | | |
| 11:6850517 | | | | | | | | | |
| 8- | | | | | | | | | |
| 68506175:- | | | | | | | | | |
| ENST00000 | BTG1 | 5′ uORF | 5′ uORF | Detected | — | — | Detected | — | — |
| 256015.4_1_ | | | | | | | | | |
| 12:9253953 | | | | | | | | | |
| 5- | | | | | | | | | |
| 92539610:- | | | | | | | | | |
| ENST00000 | ERF | Out-of- | Out-of- | Detected | — | — | Detected | — | — |
| 440177.6_1_ | | Frame | Frame | | | | | | |
| 19:4275345 | | | | | | | | | |
| 2- | | | | | | | | | |
| 42753692:- | | | | | | | | | |
| ENST00000 | N4BP2L2 | Out-of- | Out-of- | Detected | — | — | — | — | Detected |
| 267068.5_2_ | | Frame | Frame | | | | | | |
| 13:3311079 | | | | | | | | | |
| 1- | | | | | | | | | |
| 33110824:- | | | | | | | | | |
| ENST00000 | PI4KB | 5′ Overlap | 5′ Overlap | Detected | — | — | — | — | Detected |
| 368875.6_2_ | | uORF | uORF | | | | | | |
| 1:15129865 | | | | | | | | | |
| 4- | | | | | | | | | |
| 151299818:- | | | | | | | | | |
| ENST00000 | GBA | 5′ Overlap | 5′ Overlap | Detected | — | — | — | — | — |
| 327247.9_1_ | | uORF | uORF | | | | | | |
| 1:15521049 | | | | | | | | | |
| 2- | | | | | | | | | |
| 155214452:- | | | | | | | | | |
| T026338_1: | nan | lincRNA | lncRNA | — | — | — | — | — | — |
| 183227158- | | | | | | | | | |
| 183228074: | | | | | | | | | |
| + | | | | | | | | | |
| ENST00000 | ANXA2 | 3′Overlap | 3′Overlap | Detected | — | — | — | — | Detected |
| 559725.5_1_ | | dORF | dORF | | | | | | |
| 15:6065669 | | | | | | | | | |
| 2- | | | | | | | | | |
| 60682421:- | | | | | | | | | |
| ENST00000 | PTBP3 | 5′ Overlap | 5′ Overlap | — | — | — | — | — | Detected |
| 343327.6_1_ | | uORF | uORF | | | | | | |
| 9:11503045 | | | | | | | | | |
| 2- | | | | | | | | | |
| 115095785:- | | | | | | | | | |
| ENST00000 | RMND5A | Out-of- | Out-of- | Detected | — | — | — | — | Detected |
| 283632.4_1_ | | Frame | Frame | | | | | | |
| 2:86968058- | | | | | | | | | |
| 86968091:+ | | | | | | | | | |
| ENST00000 | ABHD6 | 5′ uORF | 5′ uORF | — | — | — | — | — | — |
| 478253.5_1_ | | | | | | | | | |
| 3:58223274- | | | | | | | | | |
| 58223340:+ | | | | | | | | | |
| ENST00000 | ABR | ncRNA | lncRNA | — | — | — | — | — | — |
| 571543.1_1_ | | Processed | | | | | | | |
| 17:1131443- | | Transcript | | | | | | | |
| 1132291:- | | | | | | | | | |
| ENST00000 | AC073465.3 | Pseudogene | Pseudogene | — | — | — | — | — | — |
| 440938.1_1_ | | | | | | | | | |
| 2:17435059 | | | | | | | | | |
| 5- | | | | | | | | | |
| 174351021:- | | | | | | | | | |
| ENST00000 | ACTBP7 | Pseudogene | Pseudogene | — | — | — | — | — | — |
| 418351.1_1_ | | | | | | | | | |
| 15:4428126 | | | | | | | | | |
| 1- | | | | | | | | | |
| 44281585:- | | | | | | | | | |
| ENST00000 | ALDH6A1 | ncRNA | lncRNA | — | — | — | — | — | — |
| 553814.5_1_ | | Processed | | | | | | | |
| 14:7453824 | | Transcript | | | | | | | |
| 9- | | | | | | | | | |
| 74551097:- | | | | | | | | | |
| ENST00000 | ALG14 | ncRNA | lncRNA | — | — | — | — | — | — |
| 495856.1_1_ | | Processed | | | | | | | |
| 1:95507120- | | Transcript | | | | | | | |
| 95538397:- | | | | | | | | | |
| ENST00000 | ANKRD44 | Nonesense | Other | — | — | — | — | — | — |
| 447713.1_3_ | | Mediated | | | | | | | |
| 2:19800137 | | Decay | | | | | | | |
| 4- | | | | | | | | | |
| 198175430:- | | | | | | | | | |
| ENST00000 | ATXN7L2 | 3′Overlap | 3′Overlap | — | — | — | — | — | — |
| 463678.5_1_ | | dORF | dORF | | | | | | |
| 1:11002868 | | | | | | | | | |
| 6- | | | | | | | | | |
| 110035219: | | | | | | | | | |
| + | | | | | | | | | |
| ENST00000 | C11orf84 | Out-of- | Out-of- | — | — | — | — | — | — |
| 294244.8_1_ | | Frame | Frame | | | | | | |
| 11:6359448 | | | | | | | | | |
| 7- | | | | | | | | | |
| 63594532:+ | | | | | | | | | |
| ENST00000 | C1orf35 | Out-of- | Out-of- | — | — | — | — | — | — |
| 272139.4_1_ | | Frame | Frame | | | | | | |
| 1:22828917 | | | | | | | | | |
| 1- | | | | | | | | | |
| 228290722:- | | | | | | | | | |
| ENST00000 | C5orf22 | Nonesense | Other | Detected | — | — | — | — | — |
| 511208.2_1_ | | Mediated | | | | | | | |
| 5:31532460- | | Decay | | | | | | | |
| 3 1535865:+ | | | | | | | | | |
| ENST00000 | CACTIN | Out-of- | Out-of- | — | — | — | — | — | — |
| 429344.6_1_ | | Frame | Frame | | | | | | |
| 19:3623927- | | | | | | | | | |
| 3626666:- | | | | | | | | | |
| ENST00000 | CCDC84 | 3′Overlap | 3′Overlap | — | — | — | — | — | — |
| 528088.1_1_ | | dORF | dORF | | | | | | |
| 11:1188812 | | | | | | | | | |
| 37- | | | | | | | | | |
| 118882689: | | | | | | | | | |
| + | | | | | | | | | |
| ENST00000 | CCR7 | 3′ dORF | 3′ dORF | — | — | — | — | — | — |
| 579344.1_1_ | | | | | | | | | |
| 17:3871078 | | | | | | | | | |
| 7- | | | | | | | | | |
| 38710997:- | | | | | | | | | |
| ENST00000 | CCT6P4 | Pseudogene | Pseudogene | — | — | — | — | — | — |
| 430443.1_1_ | | | | | | | | | |
| 3:19043291 | | | | | | | | | |
| 6- | | | | | | | | | |
| 190432991: | | | | | | | | | |
| + | | | | | | | | | |
| ENST00000 | CD74 | 3′ dORF | 3′ dORF | — | — | — | — | — | — |
| 518797.5_2_ | | | | | | | | | |
| 5:14978149 | | | | | | | | | |
| 3- | | | | | | | | | |
| 149781793:- | | | | | | | | | |
| ENST00000 | CDC37 | 3′Overlap | 3′Overlap | Detected | — | — | — | — | — |
| 588498.1_1_ | | dORF | dORF | | | | | | |
| 19:1050600 | | | | | | | | | |
| 0- | | | | | | | | | |
| 10506648:- | | | | | | | | | |
| ENST00000 | CHD1 | ncRNA | lncRNA | — | — | — | — | — | — |
| 508756.2_1_ | | Retained | | | | | | | |
| 5:98204705- | | Intron | | | | | | | |
| 98207835:- | | | | | | | | | |
| ENST00000 | CISD2 | Nonesense | Other | — | — | — | — | — | — |
| 574446.1_1_ | | Mediated | | | | | | | |
| 4:10379022 | | Decay | | | | | | | |
| 6- | | | | | | | | | |
| 103803968: | | | | | | | | | |
| + | | | | | | | | | |
| ENST00000 | CORO7 | ncRNA | lncRNA | — | — | — | — | — | — |
| 572125.1_1_ | | Retained | | | | | | | |
| 16:4408902- | | Intron | | | | | | | |
| 4409034:- | | | | | | | | | |
| ENST00000 | CRACR2A | 3′ dORF | 3′ dORF | — | — | — | — | — | — |
| 333750.9_1_ | | | | | | | | | |
| 12:3724520- | | | | | | | | | |
| 3753802:- | | | | | | | | | |
| ENST00000 | CTSZ | Out-of- | Out-of- | Detected | — | — | — | — | — |
| 217131.5_1_ | | Frame | Frame | | | | | | |
| 20:5757268 | | | | | | | | | |
| 8- | | | | | | | | | |
| 57576600:- | | | | | | | | | |
| ENST00000 | CYTH1 | 5′ Overlap | 5′ Overlap | — | — | — | — | — | — |
| 591095.1_1_ | | uORF | uORF | | | | | | |
| 17:7670567 | | | | | | | | | |
| 1- | | | | | | | | | |
| 76732801:- | | | | | | | | | |
| ENST00000 | CYTH2 | 5′ uORF | 5′ uORF | — | — | — | — | — | — |
| 452733.6_1_ | | | | | | | | | |
| 19:4897248 | | | | | | | | | |
| 9- | | | | | | | | | |
| 48972630:+ | | | | | | | | | |
| ENST00000 | DDX18P6 | Pseudogene | Pseudogene | — | — | — | — | — | — |
| 450119.1_1_ | | | | | | | | | |
| 10:9281337 | | | | | | | | | |
| 7- | | | | | | | | | |
| 92813905:+ | | | | | | | | | |
| ENST00000 | DDX1 | Out-of- | Out-of- | — | — | — | — | — | — |
| 381341.6_1_ | | Frame | Frame | | | | | | |
| 2:15753361- | | | | | | | | | |
| 15757371:+ | | | | | | | | | |
| ENST00000 | DDX39A | 3′Overlap | 3′Overlap | — | — | — | — | — | — |
| 587730.5_1_ | | dORF | dORF | | | | | | |
| 19:1452081 | | | | | | | | | |
| 1- | | | | | | | | | |
| 14521537:- | | | | | | | | | |
| ENST00000 | DGKZ | 3′Overlap | 3′Overlap | — | — | — | — | — | — |
| 534802.1_1_ | | dORF | dORF | | | | | | |
| 11:4640023 | | | | | | | | | |
| 4- | | | | | | | | | |
| 46401494:+ | | | | | | | | | |
| ENST00000 | DOCK8 | Nonesense | Other | Detected | — | — | — | — | — |
| 483757.5_1_ | | Mediated | | | | | | | |
| 9:273070- | | Decay | | | | | | | |
| 365624:+ | | | | | | | | | |
| ENST00000 | DRAP1 | 5′ Overlap | 5′ Overlap | Detected | — | — | — | — | — |
| 312515.6_1_ | | uORF | uORF | | | | | | |
| 11:6568684 | | | | | | | | | |
| 5- | | | | | | | | | |
| 65687846:+ | | | | | | | | | |
| ENST00000 | EEF1A1P38 | Pseudogene | Pseudogene | — | — | — | — | — | — |
| 567658.1_1_ | | | | | | | | | |
| 16:2714480 | | | | | | | | | |
| 6- | | | | | | | | | |
| 27145163:- | | | | | | | | | |
| ENST00000 | EIF2S2P3 | Pseudogene | Pseudogene | — | — | — | — | — | — |
| 428356.1_1_ | | | | | | | | | |
| 10:9442853 | | | | | | | | | |
| 1- | | | | | | | | | |
| 94429500:- | | | | | | | | | |
| ENST00000 | EPB41 | ncRNA | lncRNA | — | — | — | — | — | — |
| 460378.1_1_ | | Processed | | | | | | | |
| 1:29422780- | | Transcript | | | | | | | |
| 29423143:+ | | | | | | | | | |
| ENST00000 | EXOSC10 | ncRNA | lncRNA | — | — | — | — | — | — |
| 474216.5_1_ | | Retained | | | | | | | |
| 1:11126777- | | Intron | | | | | | | |
| 11137033:- | | | | | | | | | |
| ENST00000 | FAM109A | 5′ Overlap | 5′ Overlap | — | — | — | — | — | — |
| 549321.1_1_ | | uORF | uORF | | | | | | |
| 12:1118011 | | | | | | | | | |
| 90- | | | | | | | | | |
| 111803987:- | | | | | | | | | |
| ENST00000 | FRMD4A | ncRNA | lncRNA | — | — | — | — | — | — |
| 632314.l_l_ | | Processed | | | | | | | |
| 10:1372682 | | Transcript | | | | | | | |
| 9- | | | | | | | | | |
| 13749113:- | | | | | | | | | |
| ENST00000 | FTSJ1 | ncRNA | lncRNA | — | — | — | — | — | — |
| 490202.5_1_ | | Processed | | | | | | | |
| X:48339880- | | Transcript | | | | | | | |
| 48341236:+ | | | | | | | | | |
| ENST00000 | GPDHP72 | Pseudogene | Pseudogene | — | — | — | — | — | — |
| 638093.1_1_ | | | | | | | | | |
| 6:16647806 | | | | | | | | | |
| 2- | | | | | | | | | |
| 166478587: | | | | | | | | | |
| + | | | | | | | | | |
| ENST00000 | GATB | 3′Overlap | 3′Overlap | — | — | — | — | — | — |
| 510396.1_1_ | | dORF | dORF | | | | | | |
| 4:15259380 | | | | | | | | | |
| 8- | | | | | | | | | |
| 152594048:- | | | | | | | | | |
| ENST00000 | GGA1 | 3′ dORF | 3′ dORF | — | — | — | — | — | — |
| 343632.8_1_ | | | | | | | | | |
| 22:3802893 | | | | | | | | | |
| 5- | | | | | | | | | |
| 38029157:+ | | | | | | | | | |
| ENST00000 | GNL3 | Nonesense | Other | Detected | — | — | — | — | — |
| 492349.5_1_ | | Mediated | | | | | | | |
| 3:52720087- | | Decay | | | | | | | |
| 52722217:+ | | | | | | | | | |
| ENST00000 | GPATCH4 | 3′ dORF | 3′ dORF | — | — | — | — | — | — |
| 368232.8_1_ | | | | | | | | | |
| 1:15656477 | | | | | | | | | |
| 8- | | | | | | | | | |
| 156565066:- | | | | | | | | | |
| ENST00000 | HLA-F | 3′ dORF | 3′ dORF | — | — | — | — | — | — |
| 376861.5_1_ | | | | | | | | | |
| 6:29693814- | | | | | | | | | |
| 29694269:+ | | | | | | | | | |
| ENST00000 | HNRNPAB | 3′ dORF | 3′ dORF | — | — | — | — | — | — |
| 358344.7_1_ | | | | | | | | | |
| 5:17763759 | | | | | | | | | |
| 8- | | | | | | | | | |
| 177637649: | | | | | | | | | |
| + | | | | | | | | | |
| ENST00000 | HNRNPCP2 | Pseudogene | Pseudogene | — | — | — | — | — | — |
| 399515.2_1_ | | | | | | | | | |
| 2:19078806 | | | | | | | | | |
| 1- | | | | | | | | | |
| 190788865: | | | | | | | | | |
| + | | | | | | | | | |
| ENST00000 | HNRNPCP8 | Pseudogene | Pseudogene | — | — | — | — | — | — |
| 528764.1_1_ | | | | | | | | | |
| 11:8673566 | | | | | | | | | |
| 4- | | | | | | | | | |
| 86735814:+ | | | | | | | | | |
| ENST00000 | HNRNPH3 | ncRNA | lncRNA | — | — | — | — | — | — |
| 490442.5_1_ | | Processed | | | | | | | |
| 10:7009823 | | Transcript | | | | | | | |
| 3- | | | | | | | | | |
| 70101119:+ | | | | | | | | | |
| ENST00000 | HSP90AA2P | Pseudogene | Pseudogene | — | — | — | — | — | — |
| 530115.1_1_ | | | | | | | | | |
| 11:2791155 | | | | | | | | | |
| 1- | | | | | | | | | |
| 27912580:- | | | | | | | | | |
| ENST00000 | KCTD9 | ncRNA | lncRNA | — | — | — | — | — | — |
| 522493.5_1_ | | Processed | | | | | | | |
| 8:25297162- | | Transcript | | | | | | | |
| 25298149:- | | | | | | | | | |
| ENST00000 | KIF4A | Out-of- | Out-of- | — | — | — | — | — | — |
| 374403.3_1_ | | Frame | Frame | | | | | | |
| X:69595194- | | | | | | | | | |
| 69596002:+ | | | | | | | | | |
| ENST00000 | LPGAT1 | ncRNA | lncRNA | — | — | — | — | — | — |
| 488600.1_1_ | | Processed | | | | | | | |
| 1:21200264 | | Transcript | | | | | | | |
| 0- | | | | | | | | | |
| 212003596:- | | | | | | | | | |
| ENST00000 | MED6 | ncRNA | lncRNA | — | — | — | — | — | — |
| 556423.1_1_ | | Retained | | | | | | | |
| 14:7106331 | | Intron | | | | | | | |
| 0- | | | | | | | | | |
| 71067354:- | | | | | | | | | |
| ENST00000 | MT-ATP6 | Out-of- | Out-of- | — | — | — | — | — | — |
| 361899.2_M | | Frame | Frame | | | | | | |
| T:8530- | | | | | | | | | |
| 8572:+ | | | | | | | | | |
| ENST00000 | MT-ND2 | Out-of- | Out-of- | — | — | — | — | — | — |
| 361453.3_M | | Frame | Frame | | | | | | |
| T:5228- | | | | | | | | | |
| 5411:+ | | | | | | | | | |
| ENST00000 | MYC | 5′ uORF | 5′ uORF | — | — | — | — | — | — |
| 259523.10_1_ | | | | | | | | | |
| 8:1287477 | | | | | | | | | |
| 12- | | | | | | | | | |
| 128748060: | | | | | | | | | |
| + | | | | | | | | | |
| ENST00000 | NOLC1 | 3′ dORF | 3′ dORF | — | — | — | — | — | — |
| 488254.6_1_ | | | | | | | | | |
| 10:1039220 | | | | | | | | | |
| 35- | | | | | | | | | |
| 103922146: | | | | | | | | | |
| + | | | | | | | | | |
| ENST00000 | NONO | ncRNA | lncRNA | — | — | — | — | — | — |
| 473525.1_1_ | | Processed | | | | | | | |
| X:70516758- | | Transcript | | | | | | | |
| 70517827:+ | | | | | | | | | |
| ENST00000 | NUSAP1 | Out-of- | Out-of- | Detected | — | — | — | — | — |
| 558123.5_1_ | | Frame | Frame | | | | | | |
| 15:4164832 | | | | | | | | | |
| 0- | | | | | | | | | |
| 41650442:+ | | | | | | | | | |
| ENST00000 | PANK2 | Nonesense | Other | Detected | — | — | — | — | — |
| 336066.7_1_ | | Mediated | | | | | | | |
| 20:3870065- | | Decay | | | | | | | |
| 3891231:+ | | | | | | | | | |
| ENST00000 | PIGB | Nonesense | Other | Detected | — | — | — | — | — |
| 570059.1_1_ | | Mediated | | | | | | | |
| 15:5561143 | | Decay | | | | | | | |
| 3- | | | | | | | | | |
| 55619742:+ | | | | | | | | | |
| ENST00000 | PPP1R9B | 5′ Overlap | 5′ Overlap | — | — | — | — | — | — |
| 612501.1_1_ | | uORF | uORF | | | | | | |
| 17:4822736 | | | | | | | | | |
| 7- | | | | | | | | | |
| 48227940:- | | | | | | | | | |
| ENST00000 | PSMA1 | ncRNA | lncRNA | — | — | — | — | — | — |
| 533331.5_1_ | | Retained | | | | | | | |
| 11:1453511 | | Intron | | | | | | | |
| 0- | | | | | | | | | |
| 14539190:- | | | | | | | | | |
| ENST00000 | PTCHD4 | 5′ Overlap | 5′ Overlap | — | — | — | — | — | — |
| 339488.8_2_ | | uORF | uORF | | | | | | |
| 6:48036008- | | | | | | | | | |
| 48036398:- | | | | | | | | | |
| ENST00000 | RBM39 | Nonesense | Other | — | — | — | — | — | — |
| 403542.6_1_ | | Mediated | | | | | | | |
| 20:3432844 | | Decay | | | | | | | |
| 7- | | | | | | | | | |
| 34329909:- | | | | | | | | | |
| ENST00000 | RP11-1217F2.1 | Pseudogene | Pseudogene | — | — | — | — | — | — |
| 433031.1_1_ | | | | | | | | | |
| 7:57216803- | | | | | | | | | |
| 57217046:- | | | | | | | | | |
| ENST00000 | RP11-241F15.1 | Pseudogene | Pseudogene | Detected | — | — | — | — | — |
| 399720.4_1_ | | | | | | | | | |
| 4:49490601- | | | | | | | | | |
| 49491475:- | | | | | | | | | |
| ENST00000 | RP11-24M17.3 | Pseudogene | Pseudogene | — | — | — | — | — | — |
| 567565.3_1_ | | | | | | | | | |
| 15:7603928 | | | | | | | | | |
| 7- | | | | | | | | | |
| 76039500:+ | | | | | | | | | |
| ENST00000 | RP11-280O24.3 | Pseudogene | Pseudogene | — | — | — | — | — | — |
| 435137.1_1_ | | | | | | | | | |
| 9:14204246- | | | | | | | | | |
| 14204684:+ | | | | | | | | | |
| ENST00000 | RP11-393N4.2 | Pseudogene | Pseudogene | — | — | — | — | — | — |
| 435269.1_1_ | | | | | | | | | |
| 3:17409552 | | | | | | | | | |
| 5- | | | | | | | | | |
| 174095792: | | | | | | | | | |
| + | | | | | | | | | |
| ENST00000 | RP11-440D17.3 | lincRNA | lncRNA | Detected | — | — | — | — | — |
| 609975.1_1_ | | | | | | | | | |
| 2:96191991- | | | | | | | | | |
| 96192402:- | | | | | | | | | |
| ENST00000 | RP5-882O7.4 | Pseudogene | Pseudogene | — | — | — | — | — | — |
| 411837.1_1_ | | | | | | | | | |
| 1:45453908- | | | | | | | | | |
| 45454397:- | | | | | | | | | |
| ENST00000 | RPL10P3 | Pseudogene | Pseudogene | Detected | — | — | — | — | — |
| 431607.1_1_ | | | | | | | | | |
| 9:11994298 | | | | | | | | | |
| 3- | | | | | | | | | |
| 119943337:- | | | | | | | | | |
| ENST00000 | SPDL1 | Nonesense | Other | — | — | — | — | — | — |
| 509785.5_1_ | | Mediated | | | | | | | |
| 5:16901541 | | Decay | | | | | | | |
| 4- | | | | | | | | | |
| 169017783: | | | | | | | | | |
| + | | | | | | | | | |
| ENST00000 | SQSTM1 | 3′Overlap | 3′Overlap | — | — | — | — | — | — |
| 466342.1_1_ | | dORF | dORF | | | | | | |
| 5:17925091 | | | | | | | | | |
| 6- | | | | | | | | | |
| 179260288: | | | | | | | | | |
| + | | | | | | | | | |
| ENST00000 | STRAP | Nonesense | Other | Detected | — | — | — | — | — |
| 541731.1_1_ | | Mediated | | | | | | | |
| 12:1603550 | | Decay | | | | | | | |
| 0- | | | | | | | | | |
| 16042896:+ | | | | | | | | | |
| ENST00000 | STRIP1 | 3′Overlap | 3′Overlap | — | — | — | — | — | — |
| 473429.5_1_ | | dORF | dORF | | | | | | |
| 1:11058436 | | | | | | | | | |
| 3- | | | | | | | | | |
| 110592127: | | | | | | | | | |
| + | | | | | | | | | |
| ENST00000 | TM9SF3 | 5′ Overlap | 5′ Overlap | Detected | — | — | — | — | — |
| 371142.8_1_ | | uORF | uORF | | | | | | |
| 10:9833657 | | | | | | | | | |
| 8- | | | | | | | | | |
| 98346758:- | | | | | | | | | |
| ENST00000 | UNC13D | ncRNA | lncRNA | — | — | — | — | — | — |
| 587504.5_1_ | | Processed | | | | | | | |
| 17:7383599 | | Transcript | | | | | | | |
| 5- | | | | | | | | | |
| 73839803:- | | | | | | | | | |
| ENST00000 | USP3 | Nonesense | Other | Detected | — | — | — | — | — |
| 557884.5_1_ | | Mediated | | | | | | | |
| 15:6379681 | | Decay | | | | | | | |
| 5- | | | | | | | | | |
| 63821292:+ | | | | | | | | | |
| ENST00000 | YES1P1 | Pseudogene | Pseudogene | — | — | — | — | — | — |
| 418510.1_1_ | | | | | | | | | |
| 22:2604461 | | | | | | | | | |
| 7- | | | | | | | | | |
| 26045175:+ | | | | | | | | | |
| ENST00000 | ZC3H4 | ncRNA | lncRNA | — | — | — | — | — | — |
| 594019.5_1_ | | Processed | | | | | | | |
| 19:4758866 | | Transcript | | | | | | | |
| 0- | | | | | | | | | |
| 47615436:- | | | | | | | | | |
| ENST00000 | CD27 | 5′ Overlap | 5′ Overlap | Detected | Detected | — | — | — | — |
| 266557.3_1_ | | uORF | uORF | | | | | | |
| 12:6554080- | | | | | | | | | |
| 6554389:+ | | | | | | | | | |
| ENST00000 | ATG9A | Nonesense | Other | Detected | — | Detected | — | — | — |
| 456708.5_1_ | | Mediated | | | | | | | |
| 2:22009021 | | Decay | | | | | | | |
| 1- | | | | | | | | | |
| 220094280:- | | | | | | | | | |
| ENST00000 | RP11-488L18.4 | Pseudogene | Pseudogene | Detected | Detected | Detected | — | Detected | Detected |
| 421406.1_1_ | | | | | | | | | |
| 1:24735328 | | | | | | | | | |
| 2- | | | | | | | | | |
| 247372753:- | | | | | | | | | |
| ENST00000 | ABLIM3 | 5′ uORF | 5′ uORF | — | — | — | — | — | — |
| 326685.11_1_ | | | | | | | | | |
| 5:1485211 | | | | | | | | | |
| 29- | | | | | | | | | |
| 148521582: | | | | | | | | | |
| + | | | | | | | | | |
| ENST00000 | AC002467.7 | Antisense | lncRNA | — | — | — | — | — | — |
| 609979.1_1_ | | | | | | | | | |
| 7:10738380 | | | | | | | | | |
| 8- | | | | | | | | | |
| 107384430:- | | | | | | | | | |
| ENST00000 | AC002472.8 | Pseudogene | Pseudogene | — | — | — | — | — | — |
| 442739.1_1_ | | | | | | | | | |
| 22:2135724 | | | | | | | | | |
| 1- | | | | | | | | | |
| 21363382:- | | | | | | | | | |
| ENST00000 | AC026150.6 | Pseudogene | Pseudogene | — | — | — | — | — | — |
| 564693.1_1_ | | | | | | | | | |
| 15:3080937 | | | | | | | | | |
| 2- | | | | | | | | | |
| 30809693:+ | | | | | | | | | |
| ENST00000 | ADCY4 | 5′ Overlap | 5′ Overlap | — | — | — | — | — | — |
| 554781.5_1_ | | uORF | uORF | | | | | | |
| 14:2480045 | | | | | | | | | |
| 8- | | | | | | | | | |
| 24803934:- | | | | | | | | | |
| ENST00000 | ANKMY2 | Nonesense | Other | — | — | — | — | — | — |
| 453623.5_1_ | | Mediated | | | | | | | |
| 7:16684294- | | Decay | | | | | | | |
| 16685213:- | | | | | | | | | |
| ENST00000 | ARGHAP26 | ncRNA | lncRNA | — | — | — | — | — | — |
| 470032.1_1_ | | Processed | | | | | | | |
| 5:14241676 | | Transcript | | | | | | | |
| 8- | | | | | | | | | |
| 142421157: | | | | | | | | | |
| + | | | | | | | | | |
| ENST00000 | ARSJ | ncRNA | lncRNA | — | — | — | — | — | — |
| 503013.2_1_ | | Processed | | | | | | | |
| 4:11486743 | | Transcript | | | | | | | |
| 6- | | | | | | | | | |
| 114891654:- | | | | | | | | | |
| ENST00000 | BAIAP2-AS1 | lincRNA | lncRNA | — | — | — | — | — | — |
| 573167.2_1_ | | | | | | | | | |
| 17:7900704 | | | | | | | | | |
| 2- | | | | | | | | | |
| 79008646:- | | | | | | | | | |
| ENST00000 | BTBD2 | 3′ dORF | 3′ dORF | — | — | — | — | — | — |
| 592895.5_1_ | | | | | | | | | |
| 19:1985822- | | | | | | | | | |
| 1986326:- | | | | | | | | | |
| ENST00000 | CAPRIN2 | 5′ uORF | 5′ uORF | — | — | — | — | — | — |
| 298892.9_1_ | | | | | | | | | |
| 12:3090732 | | | | | | | | | |
| 0- | | | | | | | | | |
| 30907440:- | | | | | | | | | |
| ENST00000 | CARMIL1 | 3′Overlap | 3′Overlap | — | — | — | — | — | — |
| 635618.1_3_ | | dORF | dORF | | | | | | |
| 6:25610361- | | | | | | | | | |
| 25619808:+ | | | | | | | | | |
| ENST00000 | CBARP | 3′ dORF | 3′ dORF | — | — | — | — | — | — |
| 590083.5_1_ | | | | | | | | | |
| 19:1228816- | | | | | | | | | |
| 1229167:- | | | | | | | | | |
| ENST00000 | CD44 | 3′ dORF | 3′ dORF | — | — | — | — | — | — |
| 263398.10_1_ | | | | | | | | | |
| 11:352521 | | | | | | | | | |
| 96- | | | | | | | | | |
| 35252259:+ | | | | | | | | | |
| ENST00000 | CEP170P1 | Pseudogene | Pseudogene | — | — | — | — | — | — |
| 502249.6_1_ | | | | | | | | | |
| 4:11940152 | | | | | | | | | |
| 4- | | | | | | | | | |
| 119435218: | | | | | | | | | |
| + | | | | | | | | | |
| ENST00000 | CLDN7 | 5′ uORF | 5′ uORF | — | — | — | — | — | — |
| 573745.1_1_ | | | | | | | | | |
| 17:7165530- | | | | | | | | | |
| 7166136:- | | | | | | | | | |
| ENST00000 | DDX6 | 5′ uORF | 5′ uORF | — | — | — | — | — | — |
| 620157.4_1_ | | | | | | | | | |
| 11:1186571 | | | | | | | | | |
| 61- | | | | | | | | | |
| 118661620:- | | | | | | | | | |
| ENST00000 | DESI1 | ncRNA | lncRNA | — | — | — | — | — | — |
| 463886.1_1_ | | Processed | | | | | | | |
| 22:4199862 | | Transcript | | | | | | | |
| 8- | | | | | | | | | |
| 41998772:- | | | | | | | | | |
| ENST00000 | DNAJB1 | 3′ dORF | 3′ dORF | — | — | — | — | — | — |
| 254322.2_1_ | | | | | | | | | |
| 19:1462566 | | | | | | | | | |
| 3- | | | | | | | | | |
| 14625702:- | | | | | | | | | |
| ENST00000 | DSCR3 | 3′ dORF | 3′ dORF | — | — | — | — | — | — |
| 497493.1_1_ | | | | | | | | | |
| 21:3859747 | | | | | | | | | |
| 4- | | | | | | | | | |
| 38597519:- | | | | | | | | | |
| ENST00000 | DTX2P1- | ncRNA | lncRNA | — | — | — | — | — | — |
| 636308.1_1_ | UPK3BP1- | Processed | | | | | | | |
| 7:76607904- | PMS2P11 | Transcript | | | | | | | |
| 76634926:+ | | | | | | | | | |
| ENST00000 | EEF1A1P16 | Pseudogene | Pseudogene | — | — | — | — | — | — |
| 325349.6_1_ | | | | | | | | | |
| 12:1714401 | | | | | | | | | |
| 7- | | | | | | | | | |
| 17144188:+ | | | | | | | | | |
| ENST00000 | EEF1A1P8 | Pseudogene | Pseudogene | — | — | — | — | — | — |
| 419025.1_1_ | | | | | | | | | |
| 3:18374501 | | | | | | | | | |
| 2- | | | | | | | | | |
| 183745345:- | | | | | | | | | |
| ENST00000 | FBXO32 | 3′ dORF | 3′ dORF | — | — | — | — | — | — |
| 517956.5_1_ | | | | | | | | | |
| 8:12451464 | | | | | | | | | |
| 3- | | | | | | | | | |
| 124514769:- | | | | | | | | | |
| ENST00000 | FNDC11 | 5′ uORF | 5′ uORF | — | — | — | — | — | — |
| 615526.1_1_ | | | | | | | | | |
| 20:6218526 | | | | | | | | | |
| 2- | | | | | | | | | |
| 62185625:+ | | | | | | | | | |
| ENST00000 | FOXJ2 | 5′ uORF | 5′ uORF | — | — | — | — | — | — |
| 162391.7_1_ | | | | | | | | | |
| 12:8185349- | | | | | | | | | |
| 8185487:+ | | | | | | | | | |
| ENST00000 | FOXO3B | Pseudogene | Pseudogene | — | — | — | — | — | — |
| 395675.4_1_ | | | | | | | | | |
| 17:1857562 | | | | | | | | | |
| 4- | | | | | | | | | |
| 18576137:- | | | | | | | | | |
| ENST00000 | FUBP1 | ncRNA | lncRNA | — | — | — | — | — | — |
| 489495.5_1_ | | Processed | | | | | | | |
| 1:78411296- | | Transcript | | | | | | | |
| 78412276:- | | | | | | | | | |
| ENST00000 | FXR1 | ncRNA | lncRNA | — | — | — | — | — | — |
| 481383.1_1_ | | Retained | | | | | | | |
| 3:18068809 | | Intron | | | | | | | |
| 1- | | | | | | | | | |
| 180688984: | | | | | | | | | |
| + | | | | | | | | | |
| ENST00000 | GAPDHP61 | Pseudogene | Pseudogene | Detected | — | — | — | — | — |
| 507324.1_1_ | | | | | | | | | |
| 15:6482135 | | | | | | | | | |
| 8- | | | | | | | | | |
| 64821781:- | | | | | | | | | |
| ENST00000 | GAPDHP71 | Pseudogene | Pseudogene | — | — | — | — | — | — |
| 511530.1_1_ | | | | | | | | | |
| 5:17394061 | | | | | | | | | |
| 5- | | | | | | | | | |
| 173940888: | | | | | | | | | |
| + | | | | | | | | | |
| ENST00000 | GLS | ncRNA | lncRNA | — | — | — | — | — | — |
| 471443.1_1_ | | Processed | | | | | | | |
| 2:19179630 | | Transcript | | | | | | | |
| 9- | | | | | | | | | |
| 191797409: | | | | | | | | | |
| + | | | | | | | | | |
| ENST00000 | GNB1L | 5′ Overlap | 5′ Overlap | | — | — | — | — | — |
| 460402.5_1_ | | uORF | uORF | | | | | | |
| 22:1977623 | | | | | | | | | |
| 4- | | | | | | | | | |
| 19842274:- | | | | | | | | | |
| ENST00000 | HCG18 | Antisense | lncRNA | — | — | — | — | — | — |
| 426882.5_1_ | | | | | | | | | |
| 6:30294481- | | | | | | | | | |
| 30294724:- | | | | | | | | | |
| ENST00000 | HCP5 | Sense | Other | — | — | — | — | — | — |
| 414046.2_1_ | | Overlapping | | | | | | | |
| 6:31431445- | | | | | | | | | |
| 31431841:+ | | | | | | | | | |
| ENST00000 | HEATR3 | Out-of- | Out-of- | — | — | — | — | — | — |
| 299192.7_1_ | | Frame | Frame | | | | | | |
| 16:5010409 | | | | | | | | | |
| 2- | | | | | | | | | |
| 50104164:+ | | | | | | | | | |
| ENST00000 | HEXDC | 3′Overlap | 3′Overlap | — | — | — | — | — | — |
| 585077.1_1_ | | dORF | dORF | | | | | | |
| 17:8039790 | | | | | | | | | |
| 7- | | | | | | | | | |
| 80398237:+ | | | | | | | | | |
| ENST00000 | HNRNPA1P43 | Pseudogene | Pseudogene | — | — | — | — | — | — |
| 452680.3_1_ | | | | | | | | | |
| 1:11639959 | | | | | | | | | |
| 3- | | | | | | | | | |
| 116399968:- | | | | | | | | | |
| ENST00000 | HNRNPF | 5′ uORF | 5′ uORF | — | — | — | — | — | — |
| 544000.5_1_ | | | | | | | | | |
| 10:4389007 | | | | | | | | | |
| 8- | | | | | | | | | |
| 43892652:- | | | | | | | | | |
| ENST00000 | HSPA4 | 3′ dORF | 3′ dORF | — | — | — | — | — | — |
| 304858.6_1_ | | | | | | | | | |
| 5:13244007 | | | | | | | | | |
| 8- | | | | | | | | | |
| 132440150: | | | | | | | | | |
| + | | | | | | | | | |
| ENST00000 | ITPRIP | 5′ Overlap | 5′ Overlap | — | — | — | — | — | — |
| 337478.2_1_ | | uORF | uORF | | | | | | |
| 10:1060754 | | | | | | | | | |
| 71- | | | | | | | | | |
| 106098072:- | | | | | | | | | |
| ENST00000 | KIAA1462 | Out-of- | Out-of- | — | — | — | — | — | — |
| 375377.1_1_ | | Frame | Frame | | | | | | |
| 10:3033654 | | | | | | | | | |
| 2- | | | | | | | | | |
| 30336710:- | | | | | | | | | |
| ENST00000 | LIMK1 | ncRNA | lncRNA | — | — | — | — | — | — |
| 486361.5_1_ | | Processed | | | | | | | |
| 7:73508538- | | Transcript | | | | | | | |
| 73511422:+ | | | | | | | | | |
| ENST00000 | MACF1 | 5′ Overlap | 5′ Overlap | — | — | — | — | — | — |
| 467673.5_1_ | | uORF | uORF | | | | | | |
| 1:39571193- | | | | | | | | | |
| 39720158:+ | | | | | | | | | |
| ENST00000 | MAMSTR | 5′ Overlap | 5′ Overlap | — | — | — | — | — | — |
| 594582.1_1_ | | uORF | uORF | | | | | | |
| 19:4921852 | | | | | | | | | |
| 1- | | | | | | | | | |
| 49220230:- | | | | | | | | | |
| ENST00000 | MAP4 | ncRNA | lncRNA | — | — | — | — | — | — |
| 482752.1_1_ | | Retained | | | | | | | |
| 3:47918864- | | Intron | | | | | | | |
| 47969739:- | | | | | | | | | |
| ENST00000 | MDGA1 | 5′ uORF | 5′ uORF | — | — | — | — | — | — |
| 434837.7_1_ | | | | | | | | | |
| 6:37665242- | | | | | | | | | |
| 37665689:- | | | | | | | | | |
| ENST00000 | MEST | 5′ uORF | 5′ uORF | — | — | — | — | — | — |
| 223215.8_1_ | | | | | | | | | |
| 7:13013195 | | | | | | | | | |
| 8- | | | | | | | | | |
| 130132015: | | | | | | | | | |
| + | | | | | | | | | |
| ENST00000 | MGAT5B | 5′ uORF | 5′ uORF | — | — | — | — | — | — |
| 301618.8_1_ | | | | | | | | | |
| 17:7486473 | | | | | | | | | |
| 8- | | | | | | | | | |
| 74864864:+ | | | | | | | | | |
| ENST00000 | MRM2 | ncRNA | lncRNA | — | — | — | — | — | — |
| 467199.5_1_ | | Processed | | | | | | | |
| 7:2274759- | | Transcript | | | | | | | |
| 2281791:- | | | | | | | | | |
| ENST00000 | MYBBP1A | 5′ Overlap | 5′ Overlap | — | — | — | — | — | — |
| 570986.1_1_ | | uORF | uORF | | | | | | |
| 17:4457199- | | | | | | | | | |
| 4458649:- | | | | | | | | | |
| ENST00000 | PCK2 | 5′ Overlap | 5′ Overlap | — | — | — | — | — | — |
| 545054.6_1_ | | uORF | uORF | | | | | | |
| 14:2456358 | | | | | | | | | |
| 3- | | | | | | | | | |
| 24563937:+ | | | | | | | | | |
| ENST00000 | PHLDA2 | 3′Overlap | 3′Overlap | — | — | — | — | — | — |
| 314222.4_1_ | | dORF | dORF | | | | | | |
| 11:2949785- | | | | | | | | | |
| 2950526:- | | | | | | | | | |
| ENST00000 | PHLDB3 | 5′ Overlap | 5′ Overlap | — | — | — | — | — | — |
| 292140.9_1_ | | uORF | uORF | | | | | | |
| 19:4400812 | | | | | | | | | |
| 1- | | | | | | | | | |
| 44008847:- | | | | | | | | | |
| ENST00000 | PIP5K1C | ncRNA | lncRNA | — | — | — | — | — | — |
| 587482.1_1_ | | Processed | | | | | | | |
| 19:3649915- | | Transcript | | | | | | | |
| 3700388:- | | | | | | | | | |
| ENST00000 | PPHLN1 | Nonesense | Other | — | — | — | — | — | — |
| 552429.5_1_ | | Mediated | | | | | | | |
| 12:4272001 | | Decay | | | | | | | |
| 8- | | | | | | | | | |
| 42768464:+ | | | | | | | | | |
| ENST00000 | PPL | ncRNA | lncRNA | — | — | — | — | — | — |
| 588556.1_1_ | | Retained | | | | | | | |
| 16:4942976- | | Intron | | | | | | | |
| 4943674:- | | | | | | | | | |
| ENST00000 | PPP4R3B | ncRNA | lncRNA | — | — | — | — | — | — |
| 612688.1_1_ | | Processed | | | | | | | |
| 2:55842578- | | Transcript | | | | | | | |
| 55844368:- | | | | | | | | | |
| ENST00000 | PRKACA | 5′ uORF | 5′ uORF | — | — | — | — | — | — |
| 308677.8_1_ | | | | | | | | | |
| 19:1422847 | | | | | | | | | |
| 4- | | | | | | | | | |
| 14228519:- | | | | | | | | | |
| ENST00000 | PTK2B | ncRNA | lncRNA | — | — | — | — | — | — |
| 397497.8_2_ | | Retained | | | | | | | |
| 8:27288464- | | Intron | | | | | | | |
| 27288860:+ | | | | | | | | | |
| ENST00000 | QKI | 5′ Overlap | 5′ Overlap | — | — | — | — | — | — |
| 361758.8_1_ | | uORF | uORF | | | | | | |
| 6:16383620 | | | | | | | | | |
| 9- | | | | | | | | | |
| 163876314: | | | | | | | | | |
| + | | | | | | | | | |
| ENST00000 | RANBP1 | 5′ uORF | 5′ uORF | — | — | — | — | — | — |
| 418705.2_1_ | | | | | | | | | |
| 22:2010573 | | | | | | | | | |
| 6- | | | | | | | | | |
| 20106546:+ | | | | | | | | | |
| ENST00000 | RBMS2 | Nonesense | Other | — | — | — | — | — | — |
| 552916.5_1_ | | Mediated | | | | | | | |
| 12:5691579 | | Decay | | | | | | | |
| 5- | | | | | | | | | |
| 56963704:+ | | | | | | | | | |
| ENST00000 | RP11-12M9.4 | Pseudogene | Pseudogene | — | — | — | — | — | — |
| 423293.1_1_ | | | | | | | | | |
| 22:4147093 | | | | | | | | | |
| 1- | | | | | | | | | |
| 41471243:- | | | | | | | | | |
| ENST00000 | RP11-378G13.2 | Pseudogene | Pseudogene | — | — | — | — | — | — |
| 403475.2_1_ | | | | | | | | | |
| 6:84102419- | | | | | | | | | |
| 84102602:+ | | | | | | | | | |
| ENST00000 | RP11-402J6.3 | Pseudogene | Pseudogene | — | — | — | — | — | — |
| 321285.4_1_ | | | | | | | | | |
| 4:11348614 | | | | | | | | | |
| 3- | | | | | | | | | |
| 113486335: | | | | | | | | | |
| + | | | | | | | | | |
| ENST00000 | RP11-725P16.2 | lincRNA | lncRNA | — | — | — | — | — | — |
| 608279.1_1_ | | | | | | | | | |
| 2:13310438 | | | | | | | | | |
| 0- | | | | | | | | | |
| 133104455:- | | | | | | | | | |
| ENST00000 | RP11-77I22.4 | Pseudogene | Pseudogene | — | — | — | — | — | — |
| 550612.1_1_ | | | | | | | | | |
| 12:3101492 | | | | | | | | | |
| 3- | | | | | | | | | |
| 31015109:+ | | | | | | | | | |
| ENST00000 | RP3-432I18.1 | Pseudogene | Pseudogene | — | — | — | — | — | — |
| 546846.1_1_ | | | | | | | | | |
| 12:4798701 | | | | | | | | | |
| 1- | | | | | | | | | |
| 47987203:+ | | | | | | | | | |
| ENST00000 | SCAF11 | 5′ Overlap | 5′ Overlap | — | — | — | — | — | — |
| 369367.7_2_ | | uORF | uORF | | | | | | |
| 12:4635794 | | | | | | | | | |
| 6- | | | | | | | | | |
| 46384197:- | | | | | | | | | |
| ENST00000 | SCRIB | Out-of- | Out-of- | — | — | — | — | — | — |
| 356994.6_1_ | | Frame | Frame | | | | | | |
| 8:14489605 | | | | | | | | | |
| 6- | | | | | | | | | |
| 144897480:- | | | | | | | | | |
| ENST00000 | SRRM1 | Nonesense | Other | — | — | — | — | — | — |
| 600523.5_1_ | | Mediated | | | | | | | |
| 1:24969796- | | Decay | | | | | | | |
| 24973575:+ | | | | | | | | | |
| ENST00000 | SRRM2 | 5′ Overlap | 5′ Overlap | — | — | — | — | — | — |
| 630499.2_1_ | | uORF | uORF | | | | | | |
| 16:2802712- | | | | | | | | | |
| 2807817:+ | | | | | | | | | |
| ENST00000 | STK10 | 3′ dORF | 3′ dORF | — | — | — | — | — | — |
| 176763.9_1_ | | | | | | | | | |
| 5:17147127 | | | | | | | | | |
| 4- | | | | | | | | | |
| 171471448:- | | | | | | | | | |
| ENST00000 | TCEAL6 | 3′Overlap | 3′Overlap | — | — | — | — | — | — |
| 372774.7_2_ | | dORF | dORF | | | | | | |
| X:10139570 | | | | | | | | | |
| 4- | | | | | | | | | |
| 101395815:- | | | | | | | | | |
| ENST00000 | TM4SF19-AS1 | Antisense | lncRNA | — | — | — | — | — | — |
| 444939.1_1_ | | | | | | | | | |
| 3:19605068 | | | | | | | | | |
| 4- | | | | | | | | | |
| 196051038: | | | | | | | | | |
| + | | | | | | | | | |
| ENST00000 | TMSB10 | 3′ dORF | 3′ dORF | — | — | — | — | — | — |
| 233143.5_1_ | | | | | | | | | |
| 2:85133515- | | | | | | | | | |
| 85133596:+ | | | | | | | | | |
| ENST00000 | TPTE2P5 | Pseudogene | Pseudogene | — | — | — | — | — | — |
| 458118.5_1_ | | | | | | | | | |
| 13:4142888 | | | | | | | | | |
| 5- | | | | | | | | | |
| 41438465:- | | | | | | | | | |
| ENST00000 | TUBBP1 | Pseudogene | Pseudogene | — | — | — | — | — | — |
| 518096.1_1_ | | | | | | | | | |
| 8:30210219- | | | | | | | | | |
| 30210855:+ | | | | | | | | | |
| ENST00000 | UBA52P6 | Pseudogene | Pseudogene | — | — | — | — | — | — |
| 399822.2_1_ | | | | | | | | | |
| 9:22012279- | | | | | | | | | |
| 22012411:+ | | | | | | | | | |
| ENST00000 | YWHAZP2 | Pseudogene | Pseudogene | — | — | — | — | — | — |
| 440317.1_1_ | | | | | | | | | |
| 2:12731520 | | | | | | | | | |
| 8- | | | | | | | | | |
| 127315727:- | | | | | | | | | |
| ENST00000 | ZNF558 | ncRNA | lncRNA | — | — | — | — | — | — |
| 596172.1_1_ | | Processed | | | | | | | |
| 19:8935711- | | Transcript | | | | | | | |
| 8942974:- | | | | | | | | | |
| ENST00000 | ZSCAN18 | ncRNA | lncRNA | — | — | — | — | — | — |
| 595784.1_1_ | | Retained | | | | | | | |
| 19:5859827 | | Intron | | | | | | | |
| 1- | | | | | | | | | |
| 58598791:- | | | | | | | | | |
| TCONS_00 | linc-NBPF9-9 | lincRNA | lncRNA | — | — | — | — | — | — |
| 001132_1:1 | | | | | | | | | |
| 21138933- | | | | | | | | | |
| 121169919: | | | | | | | | | |
| + | | | | | | | | | |
| T000395_1: | nan | TUCP | Other | — | — | — | — | — | — |
| 1002569- | | | | | | | | | |
| 1003328:- | | | | | | | | | |
| T093792_13: | nan | lincRNA | lncRNA | — | — | — | — | — | — |
| 44881128- | | | | | | | | | |
| 44881524:+ | | | | | | | | | |
| T234938_22: | nan | lincRNA | lncRNA | — | — | — | — | — | — |
| 44419815- | | | | | | | | | |
| 44420103:- | | | | | | | | | |
| T236099_22: | nan | lincRNA | lncRNA | — | — | — | — | — | — |
| 50328122- | | | | | | | | | |
| 50328950:- | | | | | | | | | |
| T236101_22: | nan | lincRNA | lncRNA | — | — | — | — | — | — |
| 50328365- | | | | | | | | | |
| 50329190:- | | | | | | | | | |
| T325348_7: | nan | TUCP | Other | — | — | — | — | — | — |
| 64769928- | | | | | | | | | |
| 64770336:+ | | | | | | | | | |
| T339715_8: | nan | lincRNA | lncRNA | — | — | — | — | — | — |
| 17701253- | | | | | | | | | |
| 17705101:- | | | | | | | | | |
| T348749_8: | nan | lincRNA | lncRNA | — | — | — | — | — | — |
| 103151442- | | | | | | | | | |
| 103152410:- | | | | | | | | | |
| ENST00000 | ADAMTS4 | 3′ dORF | 3′ dORF | — | — | Detected | — | — | — |
| 367996.5_1_ | | | | | | | | | |
| 1:16116074 | | | | | | | | | |
| 2- | | | | | | | | | |
| 161160913:- | | | | | | | | | |
| ENST00000 | HELZ2 | ncRNA | lncRNA | Detected | — | — | — | Detected | — |
| 479540.5_1_ | | Processed | | | | | | | |
| 20:6220345 | | Transcript | | | | | | | |
| 6- | | | | | | | | | |
| 62204512:- | | | | | | | | | |
| ENST00000 | CCT-543D15.1 | Pseudogene | Pseudogene | — | — | — | — | — | — |
| 5672991_1_ | | | | | | | | | |
| 19:9628825- | | | | | | | | | |
| 9629221:+ | | | | | | | | | |
| ENST00000 | HNRNPCP4 | Pseudogene | Pseudogene | — | — | — | — | — | — |
| 571636.1_1_ | | | | | | | | | |
| 16:1133655 | | | | | | | | | |
| 3- | | | | | | | | | |
| 11336613:+ | | | | | | | | | |
| ENST00000 | HNRNPL | 3′Overlap | 3′Overlap | — | — | — | — | — | — |
| 597731.1_1_ | | dORF | dORF | | | | | | |
| 19:3932736 | | | | | | | | | |
| 4- | | | | | | | | | |
| 39331218:- | | | | | | | | | |
| ENST00000 | HSPD1P12 | Pseudogene | Pseudogene | — | — | — | — | — | — |
| 429127.2_1_ | | | | | | | | | |
| 12:8167969- | | | | | | | | | |
| 8168583:+ | | | | | | | | | |
| ENST00000 | HUWE1 | Out-of- | Out-of- | — | — | — | — | — | — |
| 342160.7_2_ | | Frame | Frame | | | | | | |
| X:53630437- | | | | | | | | | |
| 53631569:- | | | | | | | | | |
| ENST00000 | KSR1 | ncRNA | lncRNA | Detected | — | — | — | — | — |
| 579309.1_1_ | | Processed | | | | | | | |
| 17:2578381 | | Transcript | | | | | | | |
| 0- | | | | | | | | | |
| 25810713:+ | | | | | | | | | |
| ENST00000 | LDHA | Nonesense | Other | Detected | — | — | — | — | — |
| 536528.5_1_ | | Mediated | | | | | | | |
| 11:1841615 | | Decay | | | | | | | |
| 9- | | | | | | | | | |
| 18424398:+ | | | | | | | | | |
| ENST00000 | LGALS1 | 3′Overlap | 3′Overlap | — | — | — | — | — | — |
| 464120.1_1_ | | dORF | dORF | | | | | | |
| 22:3807167 | | | | | | | | | |
| 0- | | | | | | | | | |
| 38073073:+ | | | | | | | | | |
| ENST00000 | POTEKP | Pseudogene | Pseudogene | — | — | — | — | — | — |
| 397487.3_1_ | | | | | | | | | |
| 2:13238357 | | | | | | | | | |
| 9- | | | | | | | | | |
| 132384836: | | | | | | | | | |
| + | | | | | | | | | |
| ENST00000 | PPP1R35 | 5′ Overlap | 5′ Overlap | Detected | — | — | — | — | — |
| 487452.1_1_ | | uORF | uORF | | | | | | |
| 7:10003355 | | | | | | | | | |
| 0- | | | | | | | | | |
| 100034039:- | | | | | | | | | |
| ENST00000 | RABGAP1L | Nonesense | Other | Detected | — | — | — | — | — |
| 635248.1_1_ | | Mediated | | | | | | | |
| 1:17412872 | | Decay | | | | | | | |
| 6- | | | | | | | | | |
| 174212140: | | | | | | | | | |
| + | | | | | | | | | |
| ENST00000 | RP11-274J15.2 | Pseudogene | Pseudogene | — | — | — | — | — | — |
| 465850.1_1_ | | | | | | | | | |
| 3:11382209 | | | | | | | | | |
| 8- | | | | | | | | | |
| 113822344:- | | | | | | | | | |
| ENST00000 | RP11-511P7.6 | ncRNA | lncRNA | Detected | — | — | — | — | — |
| 498682.2_3_ | | Processed | | | | | | | |
| 7:15010468 | | Transcript | | | | | | | |
| 1- | | | | | | | | | |
| 150105308: | | | | | | | | | |
| + | | | | | | | | | |
| ENST00000 | RPL7P50 | Pseudogene | Pseudogene | — | — | — | — | — | — |
| 600588.1_1_ | | | | | | | | | |
| 19:6593418- | | | | | | | | | |
| 6593715:+ | | | | | | | | | |
| ENST00000 | SNRNP70 | Nonesense | Other | Detected | — | — | — | — | — |
| 401730.5_1_ | | Mediated | | | | | | | |
| 19:4958877 | | Decay | | | | | | | |
| 4- | | | | | | | | | |
| 49605396:+ | | | | | | | | | |
| ENST00000 | C20orf24 | Nonesense | Other | Detected | — | Detected | — | — | — |
| 483815.5_1_ | | Mediated | | | | | | | |
| 20:3523435 | | Decay | | | | | | | |
| 1- | | | | | | | | | |
| 35236338:+ | | | | | | | | | |
| ENST00000 | HNRNPUL2- | 5′ Overlap | 5′ Overlap | Detected | — | — | — | — | — |
| 403734.2_1_ | BSCL2 | uORF | uORF | | | | | | |
| 11:6249435 | | | | | | | | | |
| 9- | | | | | | | | | |
| 62494803:- | | | | | | | | | |
| ENST00000 | RPL13A | Nonesense | Other | Detected | — | — | — | — | — |
| 624069.3_1_ | | Mediated | | | | | | | |
| 19:4999087 | | Decay | | | | | | | |
| 7- | | | | | | | | | |
| 49993734:+ | | | | | | | | | |
| | MEL.5_ | MEL.6_ | MEL.7_ | MEL.11_ | MEL.15_ | OV1._ | RCC.9_ | B721_ | GBM.6_ |
| | MHCI | MHCI | MHCI | MHCI | MHCI | MHCI | MHCI | WP | WP |
| ENST00000 | — | — | — | — | — | — | — | — | — |
| 520639.1_1_ | | | | | | | | | |
| 5:15387334 | | | | | | | | | |
| 8- | | | | | | | | | |
| 153873642: | | | | | | | | | |
| + | | | | | | | | | |
| ENST00000 | — | — | — | — | — | — | — | — | — |
| 597550.5_1_ | | | | | | | | | |
| 19:5370048 | | | | | | | | | |
| 8- | | | | | | | | | |
| 53703885:+ | | | | | | | | | |
| ENST00000 | — | — | — | — | — | — | — | — | — |
| 476149.1_1_ | | | | | | | | | |
| 2:11858822 | | | | | | | | | |
| 9- | | | | | | | | | |
| 118588328: | | | | | | | | | |
| + | | | | | | | | | |
| ENST00000 | — | — | — | — | — | — | — | — | — |
| 490164.1_1_ | | | | | | | | | |
| 10:9298250 | | | | | | | | | |
| 4- | | | | | | | | | |
| 92982606:+ | | | | | | | | | |
| ENST00000 | — | — | — | — | — | — | — | — | — |
| 614189.4_1_ | | | | | | | | | |
| 10:9301111 | | | | | | | | | |
| 1- | | | | | | | | | |
| 93011153:+ | | | | | | | | | |
| ENST00000 | — | — | — | — | — | — | — | — | — |
| 376509.4_1_ | | | | | | | | | |
| X:48776104- | | | | | | | | | |
| 48776263:- | | | | | | | | | |
| ENST00000 | — | — | — | — | — | — | — | — | — |
| 513546.3_1_ | | | | | | | | | |
| 4:89199386- | | | | | | | | | |
| 89199578:- | | | | | | | | | |
| ENST00000 | — | — | — | — | — | — | — | — | — |
| 421826.6_2_ | | | | | | | | | |
| 6:27223302- | | | | | | | | | |
| 27223911:+ | | | | | | | | | |
| ENST00000 | — | — | — | — | — | — | — | — | — |
| 553247.1_1_ | | | | | | | | | |
| 12:7668760 | | | | | | | | | |
| 6- | | | | | | | | | |
| 76687717:+ | | | | | | | | | |
| ENST00000 | — | — | — | — | — | — | — | — | — |
| 588880.5_1_ | | | | | | | | | |
| 17:7513710 | | | | | | | | | |
| 5- | | | | | | | | | |
| 75137180:+ | | | | | | | | | |
| ENST00000 | — | — | — | — | — | — | — | — | — |
| 550131.5_2_ | | | | | | | | | |
| 21:3064277 | | | | | | | | | |
| 5- | | | | | | | | | |
| 30693608:+ | | | | | | | | | |
| ENST00000 | — | — | — | — | — | — | — | — | — |
| 395788.3_1_ | | | | | | | | | |
| 6:31805020- | | | | | | | | | |
| 31805074:+ | | | | | | | | | |
| ENST00000 | — | — | — | — | — | — | — | — | — |
| 466854.5_1_ | | | | | | | | | |
| 3:12208187 | | | | | | | | | |
| 3- | | | | | | | | | |
| 122084420:- | | | | | | | | | |
| ENST00000 | — | — | — | — | — | — | — | — | — |
| 396212.6_1_ | | | | | | | | | |
| 16:1953526 | | | | | | | | | |
| 1- | | | | | | | | | |
| 19535342:+ | | | | | | | | | |
| ENST00000 | — | — | — | — | — | — | — | — | — |
| 411858.1_1_ | | | | | | | | | |
| 7:6863774- | | | | | | | | | |
| 6864307:- | | | | | | | | | |
| ENST00000 | — | — | — | — | — | — | — | — | — |
| 309534.10_1_ | | | | | | | | | |
| 3:8810729 | | | | | | | | | |
| 1- | | | | | | | | | |
| 88108197:- | | | | | | | | | |
| ENST00000 | — | — | — | — | — | — | — | — | — |
| 547435.1_1_ | | | | | | | | | |
| 12:7725407 | | | | | | | | | |
| 7- | | | | | | | | | |
| 77257079:- | | | | | | | | | |
| ENST00000 | — | — | — | — | — | — | — | — | — |
| 382952.7_2_ | | | | | | | | | |
| 4:1235125- | | | | | | | | | |
| 1235221:- | | | | | | | | | |
| ENST00000 | — | — | — | — | — | — | — | — | — |
| 396185.7_1_ | | | | | | | | | |
| 3:41241009- | | | | | | | | | |
| 41266025:+ | | | | | | | | | |
| ENST00000 | — | — | — | — | — | — | — | — | — |
| 266000.10_2_ | | | | | | | | | |
| 6:3328971 | | | | | | | | | |
| 1- | | | | | | | | | |
| 33290733:- | | | | | | | | | |
| ENST00000 | — | — | — | — | — | — | — | — | — |
| 310045.7_1_ | | | | | | | | | |
| 18:6518203 | | | | | | | | | |
| 2- | | | | | | | | | |
| 65182113:- | | | | | | | | | |
| ENST00000 | — | — | — | — | — | — | — | — | — |
| 334286.7_1_ | | | | | | | | | |
| 13:7849271 | | | | | | | | | |
| 5- | | | | | | | | | |
| 78492892:- | | | | | | | | | |
| ENST00000 | — | — | — | — | — | — | — | — | — |
| 382772.3_1_ | | | | | | | | | |
| Y:22737737- | | | | | | | | | |
| 22744490:+ | | | | | | | | | |
| ENST00000 | — | — | — | — | — | — | — | — | — |
| 379904.8_1_ | | | | | | | | | |
| 5:96215484- | | | | | | | | | |
| 96215517:+ | | | | | | | | | |
| ENST00000 | — | — | — | — | — | — | — | — | — |
| 381118.7_1_ | | | | | | | | | |
| 5:60224765- | | | | | | | | | |
| 60240875:- | | | | | | | | | |
| ENST00000 | — | — | — | — | — | — | — | — | — |
| 274457.4_1_ | | | | | | | | | |
| 5:11487925 | | | | | | | | | |
| 0- | | | | | | | | | |
| 114879322:- | | | | | | | | | |
| ENST00000 | — | — | — | — | — | — | — | — | — |
| 440174.1_1_ | | | | | | | | | |
| 8:38314980- | | | | | | | | | |
| 38320646:- | | | | | | | | | |
| ENST00000 | — | — | — | — | — | — | — | — | — |
| 520817.5_1_ | | | | | | | | | |
| 8:41367338- | | | | | | | | | |
| 4136752 1 :+ | | | | | | | | | |
| ENST00000 | — | — | — | — | — | — | — | — | — |
| 396987.7_1_ | | | | | | | | | |
| 8:41455847- | | | | | | | | | |
| 41455943:+ | | | | | | | | | |
| ENST00000 | — | — | — | — | — | — | — | — | — |
| 398810.6_1_ | | | | | | | | | |
| 7:50660504- | | | | | | | | | |
| 50660642:- | | | | | | | | | |
| ENST00000 | — | — | — | — | — | — | — | — | — |
| 467204.1_1_ | | | | | | | | | |
| 1:15525233 | | | | | | | | | |
| 7- | | | | | | | | | |
| 155256975: | | | | | | | | | |
| + | | | | | | | | | |
| ENST00000 | — | — | — | — | — | — | — | — | — |
| 603731.1_2_ | | | | | | | | | |
| 7:35734066- | | | | | | | | | |
| 35735032:- | | | | | | | | | |
| ENST00000 | — | — | — | — | — | — | — | — | — |
| 499006.6_1_ | | | | | | | | | |
| 14:9843586 | | | | | | | | | |
| 3- | | | | | | | | | |
| 98444319:- | | | | | | | | | |
| ENST00000 | — | — | — | — | — | — | — | — | — |
| 557682.6_1_ | | | | | | | | | |
| 15:9342607 | | | | | | | | | |
| 2- | | | | | | | | | |
| 93426198:+ | | | | | | | | | |
| ENST00000 | — | — | — | — | — | — | — | — | — |
| 478293.1_1_ | | | | | | | | | |
| 10:1299140 | | | | | | | | | |
| 37- | | | | | | | | | |
| 129924496:- | | | | | | | | | |
| ENST00000 | — | — | — | — | — | — | — | — | |
| 510666.1_1_ | | | | | | | | | |
| 5:68840710- | | | | | | | | | |
| 68849495:+ | | | | | | | | | |
| ENST00000 | — | — | — | — | — | — | — | — | — |
| 302036.11_2 | | | | | | | | | |
| 3:9791692- | | | | | | | | | |
| 9792019:+ | | | | | | | | | |
| ENST00000 | — | — | — | — | — | — | — | — | — |
| 545260.5_2_ | | | | | | | | | |
| 10:3510404 | | | | | | | | | |
| 2- | | | | | | | | | |
| 35104177:- | | | | | | | | | |
| ENST00000 | — | — | — | — | — | — | — | — | — |
| 300146.9_1_ | | | | | | | | | |
| 11:5942678 | | | | | | | | | |
| 6- | | | | | | | | | |
| 59434427:- | | | | | | | | | |
| ENST00000 | — | — | — | — | — | — | — | — | — |
| 611950.1_1_ | | | | | | | | | |
| 5:14085578 | | | | | | | | | |
| 6- | | | | | | | | | |
| 140855918: | | | | | | | | | |
| + | | | | | | | | | |
| ENST00000 | — | — | — | — | — | — | — | — | — |
| 487475.5_1_ | | | | | | | | | |
| 3:18401707 | | | | | | | | | |
| 6- | | | | | | | | | |
| 184017693: | | | | | | | | | |
| + | | | | | | | | | |
| ENST00000 | — | — | — | — | — | — | — | — | — |
| 393203.2_1_ | | | | | | | | | |
| 1:11745273 | | | | | | | | | |
| 9- | | | | | | | | | |
| 117484357: | | | | | | | | | |
| + | | | | | | | | | |
| ENST00000 | — | — | — | — | — | — | — | — | — |
| 449182.1_1_ | | | | | | | | | |
| 7:12161261 | | | | | | | | | |
| 2- | | | | | | | | | |
| 121612669: | | | | | | | | | |
| + | | | | | | | | | |
| ENST00000 | — | — | — | — | — | — | — | — | — |
| 450506.5_1_ | | | | | | | | | |
| 1:15131841 | | | | | | | | | |
| 4- | | | | | | | | | |
| 151318804:- | | | | | | | | | |
| ENST00000 | — | — | — | — | — | — | — | — | — |
| 225430.8_2_ | | | | | | | | | |
| 17:3735654 | | | | | | | | | |
| 2- | | | | | | | | | |
| 37357495:+ | | | | | | | | | |
| ENST00000 | — | — | — | — | — | — | — | — | — |
| 416673.6_1_ | | | | | | | | | |
| 2:11436976 | | | | | | | | | |
| 3- | | | | | | | | | |
| 114384446:- | | | | | | | | | |
| ENST00000 | — | — | — | — | — | — | — | — | — |
| 444363.5_1_ | | | | | | | | | |
| 7:87488057- | | | | | | | | | |
| 87505510:- | | | | | | | | | |
| ENST00000 | — | — | — | — | — | — | — | — | — |
| 587692.5_1_ | | | | | | | | | |
| 6:86388339- | | | | | | | | | |
| 86388411:- | | | | | | | | | |
| ENST00000 | — | — | — | — | — | — | — | — | — |
| 510283.5_1_ | | | | | | | | | |
| 17:4912406 | | | | | | | | | |
| 5- | | | | | | | | | |
| 49124107:- | | | | | | | | | |
| ENST00000 | — | — | — | — | — | — | — | — | — |
| 623234.1_1_ | | | | | | | | | |
| 11:6533735 | | | | | | | | | |
| 0- | | | | | | | | | |
| 65337503:- | | | | | | | | | |
| ENST00000 | — | — | — | — | — | — | — | — | — |
| 515710.1_1_ | | | | | | | | | |
| 5:14441357- | | | | | | | | | |
| 14465986:+ | | | | | | | | | |
| ENST00000 | — | — | — | — | — | — | — | — | — |
| 482741.1_1_ | | | | | | | | | |
| 17:1870094 | | | | | | | | | |
| 0- | | | | | | | | | |
| 18702212:+ | | | | | | | | | |
| ENST00000 | — | — | — | — | — | — | — | — | — |
| 483191.5_1_ | | | | | | | | | |
| 1:16112378 | | | | | | | | | |
| 4- | | | | | | | | | |
| 161128238: | | | | | | | | | |
| + | | | | | | | | | |
| ENST00000 | — | — | — | — | — | — | — | — | — |
| 409470.5_2_ | | | | | | | | | |
| 2:85843553- | | | | | | | | | |
| 85850780:+ | | | | | | | | | |
| ENST00000 | — | — | — | — | — | — | — | — | — |
| 371544.7_1_ | | | | | | | | | |
| 1:53018618- | | | | | | | | | |
| 53018768:- | | | | | | | | | |
| ENST00000 | — | — | — | — | — | — | — | — | — |
| 469416.1_1_ | | | | | | | | | |
| 1:40916330- | | | | | | | | | |
| 40916492:+ | | | | | | | | | |
| ENST00000 | — | — | — | — | — | — | — | — | — |
| 443387.2_1_ | | | | | | | | | |
| 19:3672715 | | | | | | | | | |
| 1- | | | | | | | | | |
| 36727859:+ | | | | | | | | | |
| ENST00000 | — | — | — | — | — | — | — | — | — |
| 361524.7_1_ | | | | | | | | | |
| 18:2290214 | | | | | | | | | |
| 1- | | | | | | | | | |
| 22932115:- | | | | | | | | | |
| ENST00000 | — | — | — | — | — | — | — | — | — |
| 595189.5_1_ | | | | | | | | | |
| 19:5252094 | | | | | | | | | |
| 5- | | | | | | | | | |
| 52529004:- | | | | | | | | | |
| T028857_1: | — | — | — | — | — | — | — | — | — |
| 205417257- | | | | | | | | | |
| 205417461: | | | | | | | | | |
| + | | | | | | | | | |
| T302015_6: | — | — | — | — | — | — | — | — | — |
| 33650185- | | | | | | | | | |
| 33650641:- | | | | | | | | | |
| ENST00000 | — | — | — | — | — | — | — | — | — |
| 399959.6_1_ | | | | | | | | | |
| X:41200742- | | | | | | | | | |
| 41202586:+ | | | | | | | | | |
| ENST00000 | — | — | — | — | — | — | — | — | — |
| 558746.5_1_ | | | | | | | | | |
| 15:7916539 | | | | | | | | | |
| 3- | | | | | | | | | |
| 79172860:+ | | | | | | | | | |
| ENST00000 | — | — | — | — | — | — | — | — | — |
| 404241.6_1_ | | | | | | | | | |
| 22:3991675 | | | | | | | | | |
| 1- | | | | | | | | | |
| 39917530:+ | | | | | | | | | |
| ENST00000 | — | — | — | — | — | — | — | — | — |
| 493504.1_1_ | | | | | | | | | |
| 19:5908694 | | | | | | | | | |
| 2- | | | | | | | | | |
| 59092775:+ | | | | | | | | | |
| ENST00000 | — | — | — | — | — | — | — | — | — |
| 378037.9_1_ | | | | | | | | | |
| 13:5303070 | | | | | | | | | |
| 8- | | | | | | | | | |
| 53035053:+ | | | | | | | | | |
| ENST00000 | — | — | — | — | — | — | — | — | — |
| 261401.7_1_ | | | | | | | | | |
| 12:1090950 | | | | | | | | | |
| 90- | | | | | | | | | |
| 109125303:- | | | | | | | | | |
| ENST00000 | — | — | — | — | — | — | — | — | — |
| 282570.3_1_ | | | | | | | | | |
| 2:70056849- | | | | | | | | | |
| 70056930:+ | | | | | | | | | |
| ENST00000 | — | — | — | — | — | — | — | — | — |
| 343677.3_1_ | | | | | | | | | |
| 6:26056391- | | | | | | | | | |
| 26056457:- | | | | | | | | | |
| ENST00000 | — | — | — | — | — | — | — | — | — |
| 271843.8_1_ | | | | | | | | | |
| 1:15395000 | | | | | | | | | |
| 7- | | | | | | | | | |
| 153950046:- | | | | | | | | | |
| ENST00000 | — | — | — | — | — | — | — | — | — |
| 539253.5_1_ | | | | | | | | | |
| 1:20531279 | | | | | | | | | |
| 2- | | | | | | | | | |
| 205312870:- | | | | | | | | | |
| ENST00000 | — | — | — | — | — | — | — | — | — |
| 553932.5_1_ | | | | | | | | | |
| 14:3930725 | | | | | | | | | |
| 6- | | | | | | | | | |
| 39307550:- | | | | | | | | | |
| ENST00000 | — | — | — | — | — | — | — | — | — |
| 508090.1_1_ | | | | | | | | | |
| 5:18023591 | | | | | | | | | |
| 6- | | | | | | | | | |
| 180242424:- | | | | | | | | | |
| ENST00000 | — | — | — | — | — | — | — | — | — |
| 422154.6_1_ | | | | | | | | | |
| X:10294015 | | | | | | | | | |
| 8- | | | | | | | | | |
| 102941669:- | | | | | | | | | |
| ENST00000 | — | — | — | — | — | — | — | — | — |
| 611950.1_1_ | | | | | | | | | |
| 5:14085555 | | | | | | | | | |
| 6- | | | | | | | | | |
| 140855619: | | | | | | | | | |
| + | | | | | | | | | |
| ENST00000 | — | — | — | — | — | — | — | — | — |
| 416120.1_1_ | | | | | | | | | |
| 6:64259201- | | | | | | | | | |
| 64259360:- | | | | | | | | | |
| ENST00000 | — | — | — | — | — | — | — | — | — |
| 411466.6_3_ | | | | | | | | | |
| 16:3070955 | | | | | | | | | |
| 3- | | | | | | | | | |
| 30715400:+ | | | | | | | | | |
| ENST00000 | — | — | — | — | — | — | — | — | — |
| 270061.11_1_ | | | | | | | | | |
| 19:185303 | | | | | | | | | |
| 29- | | | | | | | | | |
| 18530365:+ | | | | | | | | | |
| ENST00000 | — | — | — | — | — | — | — | — | — |
| 233615.6_2_ | | | | | | | | | |
| 2:74685568- | | | | | | | | | |
| 74685718:+ | | | | | | | | | |
| ENST00000 | — | — | — | — | — | — | — | — | — |
| 367210.2_2_ | | | | | | | | | |
| 1:20377143 | | | | | | | | | |
| 5- | | | | | | | | | |
| 203771690: | | | | | | | | | |
| + | | | | | | | | | |
| ENST00000 | — | — | — | — | — | — | — | — | — |
| 485351.1_1_ | | | | | | | | | |
| 10:4414151 | | | | | | | | | |
| 0- | | | | | | | | | |
| 44141681:- | | | | | | | | | |
| TCONS_00 | — | — | — | — | — | — | — | — | — |
| 030037_GL | | | | | | | | | |
| 000220.1:97 | | | | | | | | | |
| 269-97317:+ | | | | | | | | | |
| ENST00000 | — | — | — | — | — | — | — | — | — |
| 505073.5_1_ | | | | | | | | | |
| 4:91048743- | | | | | | | | | |
| 91048815:+ | | | | | | | | | |
| ENST00000 | — | — | — | — | — | — | — | — | — |
| 292577.11_1_ | | | | | | | | | |
| 19:188171 | | | | | | | | | |
| 2-1885478:- | | | | | | | | | |
| ENST00000 | — | — | — | — | — | — | — | — | — |
| 418430.1_1_ | | | | | | | | | |
| 2:23799319 | | | | | | | | | |
| 4- | | | | | | | | | |
| 237993419:- | | | | | | | | | |
| ENST00000 | — | — | — | — | — | — | — | — | — |
| 312916.11_1_ | | | | | | | | | |
| 5:7632632 | | | | | | | | | |
| 3- | | | | | | | | | |
| 76326545:+ | | | | | | | | | |
| ENST00000 | — | — | — | — | — | — | — | — | — |
| 606888.2_1_ | | | | | | | | | |
| 1:14547257 | | | | | | | | | |
| 5- | | | | | | | | | |
| 145473224: | | | | | | | | | |
| + | | | | | | | | | |
| ENST00000 | — | — | — | — | — | — | — | — | — |
| 264110.6_1_ | | | | | | | | | |
| 2:17600118 | | | | | | | | | |
| 9- | | | | | | | | | |
| 176015807:- | | | | | | | | | |
| ENST00000 | — | — | — | — | — | — | — | — | — |
| 475181.1_1_ | | | | | | | | | |
| 3:11235866 | | | | | | | | | |
| 6- | | | | | | | | | |
| 112359550:- | | | | | | | | | |
| ENST00000 | — | — | — | — | — | — | — | — | — |
| 575379.1_1_ | | | | | | | | | |
| 17:7358950- | | | | | | | | | |
| 7359124:+ | | | | | | | | | |
| ENST00000 | — | — | — | — | — | — | — | — | — |
| 272133.3_1_ | | | | | | | | | |
| 1:22480409 | | | | | | | | | |
| 8- | | | | | | | | | |
| 224804761: | | | | | | | | | |
| + | | | | | | | | | |
| ENST00000 | — | — | — | — | — | — | — | — | — |
| 398073.6_1_ | | | | | | | | | |
| 12:5821689 | | | | | | | | | |
| 1- | | | | | | | | | |
| 58217005:- | | | | | | | | | |
| ENST00000 | — | — | — | — | — | — | — | — | — |
| 263360.10_1_ | | | | | | | | | |
| 11:859559 | | | | | | | | | |
| 88- | | | | | | | | | |
| 85956345:+ | | | | | | | | | |
| ENST00000 | — | — | — | — | — | — | — | — | — |
| 232905.3_1_ | | | | | | | | | |
| 3:40351265- | | | | | | | | | |
| 40352449:+ | | | | | | | | | |
| ENST00000 | — | — | — | — | — | — | — | — | — |
| 487533.1_1_ | | | | | | | | | |
| 2:9411660- | | | | | | | | | |
| 9411717:- | | | | | | | | | |
| ENST00000 | — | — | — | — | — | — | — | — | — |
| 360194.8_2_ | | | | | | | | | |
| 11:1261108 | | | | | | | | | |
| 47- | | | | | | | | | |
| 126120518: | | | | | | | | | |
| + | | | | | | | | | |
| ENST00000 | — | — | — | — | — | — | — | — | — |
| 497580.5_1_ | | | | | | | | | |
| 9:12802412 | | | | | | | | | |
| 4- | | | | | | | | | |
| 128024166: | | | | | | | | | |
| + | | | | | | | | | |
| ENST00000 | — | — | — | — | — | — | — | — | — |
| 339526.8_1_ | | | | | | | | | |
| 1:18235986 | | | | | | | | | |
| 6- | | | | | | | | | |
| 182360031:- | | | | | | | | | |
| ENST00000 | — | — | — | — | — | — | — | — | — |
| 603731.1_2_ | | | | | | | | | |
| 7:35734046- | | | | | | | | | |
| 35735162:- | | | | | | | | | |
| ENST00000 | — | — | — | — | — | — | — | — | — |
| 381086.9_1_ | | | | | | | | | |
| 7:45960389- | | | | | | | | | |
| 45960869:- | | | | | | | | | |
| ENST00000 | — | — | — | — | — | — | — | — | — |
| 568689.5_1_ | | | | | | | | | |
| 16:733528- | | | | | | | | | |
| 734236:- | | | | | | | | | |
| ENST00000 | — | — | — | — | — | — | — | — | — |
| 601423.5_2_ | | | | | | | | | |
| 19:5101093 | | | | | | | | | |
| 6- | | | | | | | | | |
| 51014415:- | | | | | | | | | |
| ENST00000 | — | — | — | — | — | — | — | — | — |
| 592055.2_2_ | | | | | | | | | |
| 19:1061068 | | | | | | | | | |
| 9- | | | | | | | | | |
| 10613773:- | | | | | | | | | |
| ENST00000 | — | — | — | — | — | — | — | — | — |
| 635956.1_1_ | | | | | | | | | |
| 17:3897438 | | | | | | | | | |
| 0- | | | | | | | | | |
| 38974494:- | | | | | | | | | |
| ENST00000 | — | — | — | — | — | — | — | — | — |
| 505030.5_1_ | | | | | | | | | |
| 5:87969013- | | | | | | | | | |
| 87969124:- | | | | | | | | | |
| ENST00000 | — | — | — | — | — | — | — | — | — |
| 302475.8_1_ | | | | | | | | | |
| 5:11263015 | | | | | | | | | |
| 0- | | | | | | | | | |
| 112630183:- | | | | | | | | | |
| ENST00000 | — | — | — | — | — | — | — | — | — |
| 267984.3_1_ | | | | | | | | | |
| 15:8129357 | | | | | | | | | |
| 1- | | | | | | | | | |
| 81293748:+ | | | | | | | | | |
| ENST00000 | — | — | — | — | — | — | — | — | — |
| 418819.5_3_ | | | | | | | | | |
| X:10293350 | | | | | | | | | |
| 1- | | | | | | | | | |
| 102939656:- | | | | | | | | | |
| ENST00000 | — | — | — | — | — | — | — | — | — |
| 439128.6_2_ | | | | | | | | | |
| 4:17053319 | | | | | | | | | |
| 0- | | | | | | | | | |
| 170533223:- | | | | | | | | | |
| ENST00000 | — | — | — | — | — | — | — | — | — |
| 277746.10_1_ | | | | | | | | | |
| 10:648930 | | | | | | | | | |
| 73- | | | | | | | | | |
| 64911919:+ | | | | | | | | | |
| ENST00000 | — | — | — | — | — | — | — | — | — |
| 540900.7_1_ | | | | | | | | | |
| 17:2844385 | | | | | | | | | |
| 8- | | | | | | | | | |
| 28499569:+ | | | | | | | | | |
| ENST00000 | — | — | — | — | — | — | — | — | — |
| 474760.1_1_ | | | | | | | | | |
| 6:10560992 | | | | | | | | | |
| 8- | | | | | | | | | |
| 105627738:- | | | | | | | | | |
| ENST00000 | — | — | — | — | — | — | — | — | — |
| 549924.5_1_ | | | | | | | | | |
| 12:5383557 | | | | | | | | | |
| 6- | | | | | | | | | |
| 53837250:+ | | | | | | | | | |
| ENST00000 | — | — | — | — | — | — | — | — | — |
| 495146.5_1_ | | | | | | | | | |
| 2:26257410- | | | | | | | | | |
| 26257569:+ | | | | | | | | | |
| ENST00000 | — | — | — | — | — | — | — | — | — |
| 604430.1_1_ | | | | | | | | | |
| 16:2933228 | | | | | | | | | |
| 5- | | | | | | | | | |
| 29370594:+ | | | | | | | | | |
| ENST00000 | — | — | — | — | — | — | — | — | — |
| 524964.1_1_ | | | | | | | | | |
| 11:1261643 | | | | | | | | | |
| 66- | | | | | | | | | |
| 126174190:- | | | | | | | | | |
| ENST00000 | — | — | — | — | — | — | — | — | — |
| 575312.1_1_ | | | | | | | | | |
| 17:7967002 | | | | | | | | | |
| 8- | | | | | | | | | |
| 79670313:- | | | | | | | | | |
| ENST00000 | — | — | — | — | — | — | — | — | — |
| 343348.10_1_ | | | | | | | | | |
| 5:1158379 | | | | | | | | | |
| 42- | | | | | | | | | |
| 115840549:- | | | | | | | | | |
| ENST00000 | — | — | — | — | — | — | — | — | — |
| 512333.1_1_ | | | | | | | | | |
| 4:77909015- | | | | | | | | | |
| 77926783:+ | | | | | | | | | |
| ENST00000 | — | — | — | — | — | — | — | — | — |
| 304987.3_1_ | | | | | | | | | |
| 11:1114732 | | | | | | | | | |
| 65- | | | | | | | | | |
| 111486986: | | | | | | | | | |
| + | | | | | | | | | |
| ENST00000 | — | — | — | — | — | — | — | — | — |
| 366630.5_1_ | | | | | | | | | |
| 1:23265123 | | | | | | | | | |
| 7- | | | | | | | | | |
| 232651315:- | | | | | | | | | |
| ENST00000 | — | — | — | — | — | — | — | — | — |
| 460013.1_1_ | | | | | | | | | |
| X:11860332 | | | | | | | | | |
| 3- | | | | | | | | | |
| 118603678: | | | | | | | | | |
| + | | | | | | | | | |
| ENST00000 | — | — | — | — | — | — | — | — | — |
| 361813.5_1_ | | | | | | | | | |
| 1:15624687 | | | | | | | | | |
| 8- | | | | | | | | | |
| 156247780:- | | | | | | | | | |
| ENST00000 | — | — | — | — | — | — | — | — | — |
| 492337.5_1_ | | | | | | | | | |
| 3:18145800 | | | | | | | | | |
| 7- | | | | | | | | | |
| 181458058: | | | | | | | | | |
| + | | | | | | | | | |
| ENST00000 | — | — | — | — | — | — | — | — | — |
| 610581.4_1_ | | | | | | | | | |
| 4:12432304 | | | | | | | | | |
| 1- | | | | | | | | | |
| 124323077: | | | | | | | | | |
| + | | | | | | | | | |
| ENST00000 | — | — | — | — | — | — | — | — | — |
| 394070.6_1_ | | | | | | | | | |
| 2:74056525- | | | | | | | | | |
| 74057988:+ | | | | | | | | | |
| ENST00000 | — | — | — | — | — | — | — | — | — |
| 635293.1_1_ | | | | | | | | | |
| 17:7579872- | | | | | | | | | |
| 7590731:- | | | | | | | | | |
| ENST00000 | — | — | — | — | — | — | — | — | — |
| 294161.10_1_ | | | | | | | | | |
| 11:624960 | | | | | | | | | |
| 07- | | | | | | | | | |
| 62496118:+ | | | | | | | | | |
| ENST00000 | — | — | — | — | — | — | — | — | — |
| 360742.9_1_ | | | | | | | | | |
| 8:33370539- | | | | | | | | | |
| 33370572:- | | | | | | | | | |
| ENST00000 | — | — | — | — | — | — | — | — | — |
| 585671.2_1_ | | | | | | | | | |
| 19:1599451- | | | | | | | | | |
| 1605445:- | | | | | | | | | |
| ENST00000 | — | — | — | — | — | — | — | — | — |
| 455732.5_1_ | | | | | | | | | |
| 2:85843288- | | | | | | | | | |
| 85850811:+ | | | | | | | | | |
| ENST00000 | — | — | — | — | — | — | — | — | — |
| 457137.6_1- | | | | | | | | | |
| 2:36924015- | | | | | | | | | |
| 36924090:+ | | | | | | | | | |
| ENST00000 | — | — | — | — | — | — | — | — | — |
| 517970.5_1_ | | | | | | | | | |
| 8:87381186- | | | | | | | | | |
| 87381258:+ | | | | | | | | | |
| ENST00000 | — | — | — | — | — | — | — | — | — |
| 565479.5_1_ | | | | | | | | | |
| 16:3010624 | | | | | | | | | |
| 9- | | | | | | | | | |
| 30106646:- | | | | | | | | | |
| ENST00000 | — | — | — | — | — | — | — | — | — |
| 488164.5_1_ | | | | | | | | | |
| 9:74975552- | | | | | | | | | |
| 74978393:- | | | | | | | | | |
| ENST00000 | — | — | — | — | — | — | — | — | — |
| 484445.5_1_ | | | | | | | | | |
| 1:40915787- | | | | | | | | | |
| 40916492:+ | | | | | | | | | |
| ENST00000 | — | — | — | — | — | — | — | — | — |
| 553891.5_1_ | | | | | | | | | |
| 14:7349113 | | | | | | | | | |
| 6- | | | | | | | | | |
| 73491307:- | | | | | | | | | |
| ENST00000 | — | — | — | — | — | — | — | — | — |
| 355972.8_1_ | | | | | | | | | |
| 20:4597665 | | | | | | | | | |
| 3- | | | | | | | | | |
| 45985423:- | | | | | | | | | |
| ENST00000 | — | — | — | — | — | — | — | — | — |
| 427117.5_1_ | | | | | | | | | |
| 19:3740732 | | | | | | | | | |
| 3- | | | | | | | | | |
| 37407440:+ | | | | | | | | | |
| T056801_11 | — | — | — | — | — | — | — | — | — |
| :32913968- | | | | | | | | | |
| 32914553:- | | | | | | | | | |
| T184311_2: | — | — | — | — | — | — | — | — | — |
| 9983205- | | | | | | | | | |
| 9983367:- | | | | | | | | | |
| ENST00000 | — | — | — | — | — | — | — | — | — |
| 328697.10_2_ | | | | | | | | | |
| 11:273844 | | | | | | | | | |
| 10- | | | | | | | | | |
| 27384560:- | | | | | | | | | |
| ENST00000 | — | — | — | — | — | — | — | — | — |
| 318443.9_1_ | | | | | | | | | |
| 15:7397677 | | | | | | | | | |
| 5- | | | | | | | | | |
| 73994724:+ | | | | | | | | | |
| ENST00000 | — | — | — | — | — | — | — | — | — |
| 246006.4_1_ | | | | | | | | | |
| 20:2306675 | | | | | | | | | |
| 8- | | | | | | | | | |
| 23066941:- | | | | | | | | | |
| ENST00000 | — | — | — | — | — | — | — | — | — |
| 264010.8_1_ | | | | | | | | | |
| 16:6760512 | | | | | | | | | |
| 0- | | | | | | | | | |
| 67644919:+ | | | | | | | | | |
| ENST00000 | — | — | — | — | — | — | — | — | — |
| 220562.8_1_ | | | | | | | | | |
| 8:28573224- | | | | | | | | | |
| 28573419:+ | | | | | | | | | |
| ENST00000 | — | — | — | — | — | — | — | — | — |
| 467337.6_1_ | | | | | | | | | |
| 17:2880824 | | | | | | | | | |
| 4- | | | | | | | | | |
| 28811254:+ | | | | | | | | | |
| ENST00000 | — | — | — | — | — | — | — | — | — |
| 515608.5_1_ | | | | | | | | | |
| 8:6484738- | | | | | | | | | |
| 6484828:- | | | | | | | | | |
| ENST00000 | — | — | — | — | — | — | — | — | — |
| 391611.6_2_ | | | | | | | | | |
| 14:6057450 | | | | | | | | | |
| 8- | | | | | | | | | |
| 60574697:+ | | | | | | | | | |
| ENST00000 | — | — | — | — | — | — | — | — | — |
| 262646.11_1_ | | | | | | | | | |
| 8:6148466 | | | | | | | | | |
| 7- | | | | | | | | | |
| 61496833:+ | | | | | | | | | |
| ENST00000 | — | — | — | — | — | — | — | — | — |
| 555835.2_2_ | | | | | | | | | |
| 14:2115277 | | | | | | | | | |
| 8- | | | | | | | | | |
| 21152838:+ | | | | | | | | | |
| ENST00000 | — | — | — | — | — | — | — | — | — |
| 484231.1_1_ | | | | | | | | | |
| 3:10000059 | | | | | | | | | |
| 5- | | | | | | | | | |
| 100000643: | | | | | | | | | |
| + | | | | | | | | | |
| ENST00000 | — | — | — | — | — | — | — | — | — |
| 347770.8_1_ | | | | | | | | | |
| 8:10387027 | | | | | | | | | |
| 3- | | | | | | | | | |
| 103876088:- | | | | | | | | | |
| ENST00000 | — | — | — | — | — | — | — | — | — |
| 428135.7_1_ | | | | | | | | | |
| 7:17979950- | | | | | | | | | |
| 17980106:- | | | | | | | | | |
| ENST00000 | — | — | — | — | — | — | — | — | — |
| 265990.10_1_ | | | | | | | | | |
| 10:936836 | | | | | | | | | |
| 79- | | | | | | | | | |
| 93683796:+ | | | | | | | | | |
| ENST00000 | — | — | — | — | — | — | — | — | — |
| 471964.5_1_ | | | | | | | | | |
| 3:15674116- | | | | | | | | | |
| 15674227:+ | | | | | | | | | |
| ENST00000 | — | — | — | — | — | — | — | — | — |
| 618522.4_1_ | | | | | | | | | |
| 11:1118530 | | | | | | | | | |
| 07- | | | | | | | | | |
| 111889757: | | | | | | | | | |
| + | | | | | | | | | |
| ENST00000 | — | — | — | — | — | — | — | — | — |
| 620860.4_1_ | | | | | | | | | |
| 1:45446712- | | | | | | | | | |
| 45452201:- | | | | | | | | | |
| ENST00000 | — | — | — | — | — | — | — | — | — |
| 369754.7_2_ | | | | | | | | | |
| 6:82461791- | | | | | | | | | |
| 82462216:- | | | | | | | | | |
| ENST00000 | — | — | — | — | — | — | — | — | — |
| 540839.7_3_ | | | | | | | | | |
| 16:6756354 | | | | | | | | | |
| 7- | | | | | | | | | |
| 67572402:+ | | | | | | | | | |
| ENST00000 | — | — | — | — | — | — | — | — | — |
| 445210.1_1_ | | | | | | | | | |
| 2:17042858 | | | | | | | | | |
| 3- | | | | | | | | | |
| 170429512:- | | | | | | | | | |
| ENST00000 | — | — | — | — | — | — | — | — | — |
| 449733.7_1_ | | | | | | | | | |
| 9:12404472 | | | | | | | | | |
| 5- | | | | | | | | | |
| 124044779: | | | | | | | | | |
| + | | | | | | | | | |
| ENST00000 | — | — | — | — | — | — | — | — | — |
| 396081.5_1_ | | | | | | | | | |
| 7:35734046- | | | | | | | | | |
| 35734373:- | | | | | | | | | |
| ENST00000 | — | — | — | — | — | — | — | — | — |
| 634733.1_1_ | | | | | | | | | |
| 6:26224458- | | | | | | | | | |
| 26225162:+ | | | | | | | | | |
| ENST00000 | — | — | — | — | — | — | — | — | — |
| 572179.5_1_ | | | | | | | | | |
| 17:3623679- | | | | | | | | | |
| 3627129:- | | | | | | | | | |
| ENST00000 | — | — | — | — | — | — | — | — | — |
| 316077.13_1_ | | | | | | | | | |
| X:1353335 | | | | | | | | | |
| 16- | | | | | | | | | |
| 135333558:- | | | | | | | | | |
| ENST00000 | — | — | — | — | — | — | — | — | — |
| 502869.5_2_ | | | | | | | | | |
| 4:71768157- | | | | | | | | | |
| 71808564:+ | | | | | | | | | |
| ENST00000 | — | — | — | — | — | — | — | — | — |
| 535664.5_1_ | | | | | | | | | |
| 12:6572068 | | | | | | | | | |
| 6- | | | | | | | | | |
| 65847531:+ | | | | | | | | | |
| ENST00000 | — | — | — | — | — | — | — | — | — |
| 381120.7_1_ | | | | | | | | | |
| 13:2801456 | | | | | | | | | |
| 9- | | | | | | | | | |
| 28024670:- | | | | | | | | | |
| ENST00000 | — | — | — | — | — | — | — | — | — |
| 430580.6_1_ | | | | | | | | | |
| 1:16939836- | | | | | | | | | |
| 16939869:- | | | | | | | | | |
| ENST00000 | — | — | — | — | — | — | — | — | — |
| 452404.6_1_ | | | | | | | | | |
| 3:19666630 | | | | | | | | | |
| 1- | | | | | | | | | |
| 196669112:- | | | | | | | | | |
| ENST00000 | — | — | — | — | — | — | — | — | — |
| 393249.6_1_ | | | | | | | | | |
| 12:7679365 | | | | | | | | | |
| 4- | | | | | | | | | |
| 76804396:- | | | | | | | | | |
| ENST00000 | — | — | — | — | — | — | — | — | — |
| 624931.1_1_ | | | | | | | | | |
| 11:8286823 | | | | | | | | | |
| 8- | | | | | | | | | |
| 82868709:+ | | | | | | | | | |
| ENST00000 | — | — | — | — | — | — | — | — | — |
| 610387.4_2_ | | | | | | | | | |
| 22:1856076 | | | | | | | | | |
| 1- | | | | | | | | | |
| 18561111:+ | | | | | | | | | |
| ENST00000 | — | — | — | — | — | — | — | — | — |
| 550590.5_1_ | | | | | | | | | |
| 12:5635214 | | | | | | | | | |
| 9- | | | | | | | | | |
| 56359825:- | | | | | | | | | |
| ENST00000 | — | — | — | — | — | — | — | — | — |
| 353107.7_1_ | | | | | | | | | |
| 8:10116284 | | | | | | | | | |
| 1- | | | | | | | | | |
| 101162934: | | | | | | | | | |
| + | | | | | | | | | |
| ENST00000 | — | — | — | — | — | — | — | — | — |
| 473764.5_1_ | | | | | | | | | |
| X:48755552- | | | | | | | | | |
| 48755627:+ | | | | | | | | | |
| ENST00000 | — | — | — | — | — | — | — | — | — |
| 379066.5_1_ | | | | | | | | | |
| 2:37544226- | | | | | | | | | |
| 37544932:- | | | | | | | | | |
| ENST00000 | — | — | — | — | — | — | — | — | — |
| 404125.5_1_ | | | | | | | | | |
| 2:54176390- | | | | | | | | | |
| 54176420:- | | | | | | | | | |
| ENST00000 | — | — | — | — | — | — | — | — | — |
| 371953.7_1_ | | | | | | | | | |
| 10:8962346 | | | | | | | | | |
| 5- | | | | | | | | | |
| 89623600:+ | | | | | | | | | |
| ENST00000 | — | — | — | — | — | — | — | — | — |
| 566783.1_1_ | | | | | | | | | |
| 16:2317143- | | | | | | | | | |
| 2317218:- | | | | | | | | | |
| ENST00000 | — | — | — | — | — | — | — | — | — |
| 380722.2_1_ | | | | | | | | | |
| 14:8599606 | | | | | | | | | |
| 0- | | | | | | | | | |
| 85996315:- | | | | | | | | | |
| ENST00000 | — | — | — | — | — | — | — | — | — |
| 633848.1_1_ | | | | | | | | | |
| 12:9808558- | | | | | | | | | |
| 9809555:+ | | | | | | | | | |
| ENST00000 | — | — | — | — | — | — | — | — | — |
| 506368.5_2_ | | | | | | | | | |
| 4:13001434 | | | | | | | | | |
| 0- | | | | | | | | | |
| 130014460:- | | | | | | | | | |
| ENST00000 | — | — | — | — | — | — | — | — | — |
| 369452.8_1_ | | | | | | | | | |
| 10:1127239 | | | | | | | | | |
| 43- | | | | | | | | | |
| 112724024: | | | | | | | | | |
| + | | | | | | | | | |
| ENST00000 | — | — | — | — | — | — | — | — | — |
| 316902.11_1_ | | | | | | | | | |
| 14:236521 | | | | | | | | | |
| 48- | | | | | | | | | |
| 23652238:- | | | | | | | | | |
| ENST00000 | — | — | — | — | — | — | — | — | — |
| 294008.3_1_ | | | | | | | | | |
| 16:3659441- | | | | | | | | | |
| 3659555:- | | | | | | | | | |
| ENST00000 | — | — | — | — | — | — | — | — | — |
| 591956.1_1_ | | | | | | | | | |
| 17:7455387 | | | | | | | | | |
| 2- | | | | | | | | | |
| 74557410:+ | | | | | | | | | |
| ENST00000 | — | — | — | — | — | — | — | — | — |
| 231061.8_1_ | | | | | | | | | |
| 5:15105421 | | | | | | | | | |
| 8- | | | | | | | | | |
| 151066527:- | | | | | | | | | |
| ENST00000 | — | — | — | — | — | — | — | — | — |
| 269033.7_1_ | | | | | | | | | |
| 17:2801164 | | | | | | | | | |
| 3- | | | | | | | | | |
| 28257046:- | | | | | | | | | |
| ENST00000 | — | — | — | — | — | — | — | — | — |
| 589434.5_1_ | | | | | | | | | |
| 2:17938814 | | | | | | | | | |
| 4- | | | | | | | | | |
| 179403504: | | | | | | | | | |
| + | | | | | | | | | |
| ENST00000 | — | — | — | — | — | — | — | — | — |
| 440408.5_1_ | | | | | | | | | |
| Y:14798457- | | | | | | | | | |
| 14798529:+ | | | | | | | | | |
| ENST00000 | — | — | — | — | — | — | — | — | — |
| 415248.1_1_ | | | | | | | | | |
| 19:5794691 | | | | | | | | | |
| 9- | | | | | | | | | |
| 57954712:+ | | | | | | | | | |
| ENST00000 | — | — | — | — | — | — | — | — | — |
| 336838.10_1_ | | | | | | | | | |
| 2:5564627 | | | | | | | | | |
| 9- | | | | | | | | | |
| 55646603:- | | | | | | | | | |
| ENST00000 | — | — | — | — | — | — | — | — | — |
| 296088.11_1_ | | | | | | | | | |
| 3:4334127 | | | | | | | | | |
| 4- | | | | | | | | | |
| 43344646:+ | | | | | | | | | |
| ENST00000 | — | — | — | — | — | — | — | — | — |
| 424913.5_1_ | | | | | | | | | |
| 3:17677167 | | | | | | | | | |
| 3- | | | | | | | | | |
| 176816307:- | | | | | | | | | |
| ENST00000 | — | — | — | — | — | — | — | — | — |
| 403825.7_1_ | | | | | | | | | |
| 7:11209054 | | | | | | | | | |
| 1- | | | | | | | | | |
| 112090697: | | | | | | | | | |
| + | | | | | | | | | |
| ENST00000 | — | — | — | — | — | — | — | — | — |
| 264221.6_2_ | | | | | | | | | |
| 4:57307918- | | | | | | | | | |
| 57312926:+ | | | | | | | | | |
| ENST00000 | — | — | — | — | — | — | — | — | — |
| 223127.7_2_ | | | | | | | | | |
| 7:10086069 | | | | | | | | | |
| 5- | | | | | | | | | |
| 100860860:- | | | | | | | | | |
| ENST00000 | — | — | — | — | — | — | — | — | — |
| 427191.6_1_ | | | | | | | | | |
| 4:87515508- | | | | | | | | | |
| 87515601:+ | | | | | | | | | |
| ENST00000 | Detected | — | — | — | — | — | — | — | — |
| 315215.11_1_ | | | | | | | | | |
| 8:3765452 | | | | | | | | | |
| 7- | | | | | | | | | |
| 37654569:+ | | | | | | | | | |
| ENST00000 | Detected | — | — | — | — | — | — | — | — |
| 428149.6_1_ | | | | | | | | | |
| 11:1074363 | | | | | | | | | |
| 94- | | | | | | | | | |
| 107436448:- | | | | | | | | | |
| ENST00000 | Detected | — | — | — | — | — | — | — | — |
| 553251.5_1_ | | | | | | | | | |
| 12:1104753 | | | | | | | | | |
| 15- | | | | | | | | | |
| 110475375: | | | | | | | | | |
| + | | | | | | | | | |
| ENST00000 | Detected | — | — | — | — | — | — | — | — |
| 429686.5_1_ | | | | | | | | | |
| 11:7242335 | | | | | | | | | |
| 8- | | | | | | | | | |
| 72423549:- | | | | | | | | | |
| ENST00000 | Detected | — | — | — | — | — | — | — | — |
| 430799.7_2_ | | | | | | | | | |
| 1:27056322- | | | | | | | | | |
| 27059215:+ | | | | | | | | | |
| ENST00000 | Detected | — | — | — | — | — | — | — | — |
| 298875.8_1_ | | | | | | | | | |
| 14:9258837 | | | | | | | | | |
| 2- | | | | | | | | | |
| 92588465:+ | | | | | | | | | |
| ENST00000 | Detected | — | — | — | — | — | — | — | — |
| 585860.2_3_ | | | | | | | | | |
| 19:3765657 | | | | | | | | | |
| 9- | | | | | | | | | |
| 37660760:- | | | | | | | | | |
| ENST00000 | Detected | — | — | — | — | — | — | — | — |
| 431489.5_1_ | | | | | | | | | |
| 2:62932928- | | | | | | | | | |
| 62934072:+ | | | | | | | | | |
| ENST00000 | Detected | — | — | — | — | — | — | — | — |
| 367114.7_2_ | | | | | | | | | |
| 1:20678574 | | | | | | | | | |
| 1- | | | | | | | | | |
| 206785795:- | | | | | | | | | |
| ENST00000 | Detected | — | — | — | — | — | — | — | — |
| 313766.5_7: | | | | | | | | | |
| 193007- | | | | | | | | | |
| 193082:+ | | | | | | | | | |
| ENST00000 | Detected | — | — | — | — | — | — | — | — |
| 353258.7_1_ | | | | | | | | | |
| 16:1953322 | | | | | | | | | |
| 8- | | | | | | | | | |
| 19533372:- | | | | | | | | | |
| ENST00000 | Detected | — | — | — | — | — | — | — | — |
| 479540.5_1_ | | | | | | | | | |
| 20:6220408 | | | | | | | | | |
| 5- | | | | | | | | | |
| 62204322:- | | | | | | | | | |
| ENST00000 | Detected | — | — | — | — | — | — | — | — |
| 401473.7_1_ | | | | | | | | | |
| 6:34204671- | | | | | | | | | |
| 34208558:+ | | | | | | | | | |
| ENST00000 | Detected | — | — | — | — | — | — | — | — |
| 296181.8_1_ | | | | | | | | | |
| 3:12457825 | | | | | | | | | |
| 8- | | | | | | | | | |
| 124592335:- | | | | | | | | | |
| ENST00000 | Detected | — | — | — | — | — | — | — | — |
| 520322.1_1_ | | | | | | | | | |
| 5:16967558 | | | | | | | | | |
| 8- | | | | | | | | | |
| 169675669:- | | | | | | | | | |
| ENST00000 | Detected | — | — | — | — | — | — | — | — |
| 588134.1_1_ | | | | | | | | | |
| 18:5344711 | | | | | | | | | |
| 6- | | | | | | | | | |
| 53447923:- | | | | | | | | | |
| ENST00000 | Detected | — | — | — | — | — | — | — | — |
| 319264.3_1_ | | | | | | | | | |
| 9:12775157- | | | | | | | | | |
| 12775265:+ | | | | | | | | | |
| ENST00000 | Detected | — | — | — | — | — | — | — | — |
| 264036.5_1_ | | | | | | | | | |
| 11:1191859 | | | | | | | | | |
| 54- | | | | | | | | | |
| 119187791:- | | | | | | | | | |
| ENST00000 | Detected | — | — | — | — | — | — | — | — |
| 373930.3_1_ | | | | | | | | | |
| 9:12347665 | | | | | | | | | |
| 9- | | | | | | | | | |
| 123476740:- | | | | | | | | | |
| ENST00000 | Detected | — | — | — | — | — | — | — | — |
| 267984.3_1_ | | | | | | | | | |
| 15:8129338 | | | | | | | | | |
| 5- | | | | | | | | | |
| 81293562:+ | | | | | | | | | |
| ENST00000 | Detected | — | — | — | — | — | — | — | — |
| 306061.10_1_ | | | | | | | | | |
| 16:566603 | | | | | | | | | |
| 90- | | | | | | | | | |
| 56660912:+ | | | | | | | | | |
| ENST00000 | Detected | — | — | — | — | — | — | — | — |
| 557644.5_1_ | | | | | | | | | |
| 12:9733044 | | | | | | | | | |
| 1- | | | | | | | | | |
| 97330501:+ | | | | | | | | | |
| ENST00000 | Detected | — | — | — | — | — | — | — | — |
| 327111.7_1_ | | | | | | | | | |
| 5:92919776- | | | | | | | | | |
| 92920418:+ | | | | | | | | | |
| ENST00000 | Detected | — | — | — | — | — | — | — | — |
| 173229.6_1_ | | | | | | | | | |
| 17:9144472- | | | | | | | | | |
| 9144925:+ | | | | | | | | | |
| ENST00000 | Detected | — | — | — | — | — | — | — | — |
| 476917.5_1_ | | | | | | | | | |
| 10:1261005 | | | | | | | | | |
| 59- | | | | | | | | | |
| 126100658:- | | | | | | | | | |
| ENST00000 | Detected | — | — | — | — | — | — | — | — |
| 372409.7_1_ | | | | | | | | | |
| 20:4456334 | | | | | | | | | |
| 6- | | | | | | | | | |
| 44566100:+ | | | | | | | | | |
| ENST00000 | Detected | — | — | — | — | — | — | — | — |
| 376327.5_1_ | | | | | | | | | |
| X:49028321- | | | | | | | | | |
| 49029491:+ | | | | | | | | | |
| ENST00000 | Detected | — | — | — | — | — | — | — | — |
| 379742.4_1_ | | | | | | | | | |
| 13:3816626 | | | | | | | | | |
| 6- | | | | | | | | | |
| 38172793:- | | | | | | | | | |
| ENST00000 | Detected | — | — | — | — | — | — | — | — |
| 329286.6_1_ | | | | | | | | | |
| 18:8609717- | | | | | | | | | |
| 8609897:+ | | | | | | | | | |
| ENST00000 | Detected | — | — | — | — | — | — | — | — |
| 465226.1_1_ | | | | | | | | | |
| 20:388311- | | | | | | | | | |
| 389346:+ | | | | | | | | | |
| ENST00000 | Detected | — | — | — | — | — | — | — | — |
| 513185.1_1_ | | | | | | | | | |
| 5:98109417- | | | | | | | | | |
| 98109651:+ | | | | | | | | | |
| ENST00000 | Detected | — | — | — | — | — | — | — | — |
| 257700.6_1_ | | | | | | | | | |
| 7:10517268 | | | | | | | | | |
| 2- | | | | | | | | | |
| 105177020: | | | | | | | | | |
| + | | | | | | | | | |
| ENST00000 | Detected | — | — | — | — | — | — | — | — |
| 436010.6_1_ | | | | | | | | | |
| 3:79068249- | | | | | | | | | |
| 79068504:- | | | | | | | | | |
| ENST00000 | Detected | — | — | — | — | — | — | — | — |
| 598273.5_1_ | | | | | | | | | |
| 19:4828414 | | | | | | | | | |
| 5- | | | | | | | | | |
| 48284407:+ | | | | | | | | | |
| ENST00000 | Detected | — | — | — | — | — | — | — | — |
| 455843.5_1_ | | | | | | | | | |
| 22:1970567 | | | | | | | | | |
| 9- | | | | | | | | | |
| 19706159:+ | | | | | | | | | |
| ENST00000 | Detected | — | — | — | — | — | — | — | — |
| 338244.5_1_ | | | | | | | | | |
| 20:4913325- | | | | | | | | | |
| 4913352:- | | | | | | | | | |
| ENST00000 | Detected | — | — | — | — | — | — | — | — |
| 519973.5_1_ | | | | | | | | | |
| 8:23386398- | | | | | | | | | |
| 23386443:+ | | | | | | | | | |
| ENST00000 | Detected | — | — | — | — | — | — | — | — |
| 396526.71 | | | | | | | | | |
| 14:3507484 | | | | | | | | | |
| 1- | | | | | | | | | |
| 35077276:- | | | | | | | | | |
| ENST00000 | Detected | — | — | — | — | — | — | — | — |
| 494062.2_1_ | | | | | | | | | |
| 13:3690987 | | | | | | | | | |
| 3- | | | | | | | | | |
| 36920564:- | | | | | | | | | |
| ENST00000 | Detected | — | — | — | — | — | — | — | — |
| 610401.4_1_ | | | | | | | | | |
| 1:54871875- | | | | | | | | | |
| 54872031:- | | | | | | | | | |
| ENST00000 | Detected | — | — | — | — | — | — | — | — |
| 275767.3_1_ | | | | | | | | | |
| 7:13483282 | | | | | | | | | |
| 9- | | | | | | | | | |
| 134849312: | | | | | | | | | |
| + | | | | | | | | | |
| ENST00000 | Detected | — | — | — | — | — | — | — | — |
| 557636.5_1_ | | | | | | | | | |
| 14:7624962 | | | | | | | | | |
| 2- | | | | | | | | | |
| 76249835:+ | | | | | | | | | |
| ENST00000 | Detected | — | — | — | — | — | — | — | — |
| 251412.7_1_ | | | | | | | | | |
| 17:4081132 | | | | | | | | | |
| 9- | | | | | | | | | |
| 40811476:+ | | | | | | | | | |
| ENST00000 | Detected | — | — | — | — | — | — | — | — |
| 319394.7_1_ | | | | | | | | | |
| 1:27633464- | | | | | | | | | |
| 27633656:+ | | | | | | | | | |
| ENST00000 | Detected | — | — | — | — | — | — | — | — |
| 556143.5_1_ | | | | | | | | | |
| 14:7349139 | | | | | | | | | |
| 2- | | | | | | | | | |
| 73491458:- | | | | | | | | | |
| ENST00000 | Detected | — | — | — | — | — | — | — | — |
| 599328.1_1_ | | | | | | | | | |
| 19:5374464 | | | | | | | | | |
| 9- | | | | | | | | | |
| 53744913:- | | | | | | | | | |
| ENST00000 | Detected | — | — | — | — | — | — | — | — |
| 397133.2_1_ | | | | | | | | | |
| 17:3572117- | | | | | | | | | |
| 3572156:+ | | | | | | | | | |
| ENST00000 | Detected | — | — | — | — | — | — | — | — |
| 559705.1_1_ | | | | | | | | | |
| 15:6438589 | | | | | | | | | |
| 9- | | | | | | | | | |
| 64386013:- | | | | | | | | | |
| ENST00000 | Detected | — | — | — | — | — | — | — | — |
| 522841.6_1_ | | | | | | | | | |
| 16:2148107 | | | | | | | | | |
| 5- | | | | | | | | | |
| 21531198:- | | | | | | | | | |
| ENST00000 | Detected | — | — | — | — | — | — | — | — |
| 497571.5_1_ | | | | | | | | | |
| 10:3824338- | | | | | | | | | |
| 3827372:- | | | | | | | | | |
| ENST00000 | Detected | — | — | — | — | — | — | — | — |
| 489392.1_1_ | | | | | | | | | |
| 9:13621644 | | | | | | | | | |
| 8- | | | | | | | | | |
| 136216496: | | | | | | | | | |
| + | | | | | | | | | |
| ENST00000 | Detected | — | — | — | — | — | — | — | — |
| 550146.5_1_ | | | | | | | | | |
| 12:4835737 | | | | | | | | | |
| 4- | | | | | | | | | |
| 48358078:+ | | | | | | | | | |
| ENST00000 | Detected | — | — | — | — | — | — | — | — |
| 294309.7_2_ | | | | | | | | | |
| 11:6882152 | | | | | | | | | |
| 7- | | | | | | | | | |
| 68822650:+ | | | | | | | | | |
| ENST00000 | Detected | — | — | — | — | — | — | — | — |
| 374647.9_1_ | | | | | | | | | |
| 9:11169334 | | | | | | | | | |
| 3- | | | | | | | | | |
| 111696326:- | | | | | | | | | |
| ENST00000 | Detected | — | — | — | — | — | — | — | — |
| 537473.2_1_ | | | | | | | | | |
| 12:5706554 | | | | | | | | | |
| 9- | | | | | | | | | |
| 57066549:- | | | | | | | | | |
| ENST00000 | Detected | — | — | — | — | — | — | — | — |
| 312814.10_1_ | | | | | | | | | |
| 7:1124303 | | | | | | | | | |
| 70- | | | | | | | | | |
| 112430523:- | | | | | | | | | |
| ENST00000 | Detected | — | — | — | — | — | — | — | — |
| 206423.7_2_ | | | | | | | | | |
| 3:11235940 | | | | | | | | | |
| 9- | | | | | | | | | |
| 112359550:- | | | | | | | | | |
| ENST00000 | — | Detected | — | — | — | — | — | — | — |
| 554410.5_1_ | | | | | | | | | |
| 14:3296347 | | | | | | | | | |
| 9- | | | | | | | | | |
| 32963518:+ | | | | | | | | | |
| ENST00000 | — | Detected | — | — | — | — | — | — | — |
| 448903.6_2_ | | | | | | | | | |
| 11:926043- | | | | | | | | | |
| 959460:+ | | | | | | | | | |
| ENST00000 | — | Detected | — | — | — | — | — | — | — |
| 378119.8_1_ | | | | | | | | | |
| 6:24719560- | | | | | | | | | |
| 24720079:- | | | | | | | | | |
| ENST00000 | — | Detected | — | — | — | — | — | — | — |
| 620897.4_2_ | | | | | | | | | |
| 7:13485109 | | | | | | | | | |
| 6- | | | | | | | | | |
| 134851354:- | | | | | | | | | |
| ENST00000 | — | Detected | — | — | — | — | — | — | — |
| 395850.7_1_ | | | | | | | | | |
| 11:3373182 | | | | | | | | | |
| 0- | | | | | | | | | |
| 33743981:- | | | | | | | | | |
| ENST00000 | — | Detected | — | — | — | — | — | — | — |
| 566182.1_1_ | | | | | | | | | |
| 16:6696843 | | | | | | | | | |
| 4- | | | | | | | | | |
| 66968817:+ | | | | | | | | | |
| ENST00000 | — | Detected | — | — | — | — | — | — | — |
| 539028.1_1_ | | | | | | | | | |
| 12:1001009 | | | | | | | | | |
| 8- | | | | | | | | | |
| 10010200:- | | | | | | | | | |
| ENST00000 | — | Detected | — | — | — | — | — | — | — |
| 624602.1_1_ | | | | | | | | | |
| 5:18023750 | | | | | | | | | |
| 7- | | | | | | | | | |
| 180237573: | | | | | | | | | |
| + | | | | | | | | | |
| ENST00000 | — | Detected | — | — | — | — | — | — | — |
| 453024.5_1_ | | | | | | | | | |
| 3:41241021- | | | | | | | | | |
| 41265725:+ | | | | | | | | | |
| ENST00000 | — | Detected | — | — | — | — | — | — | — |
| 382247.5_1_ | | | | | | | | | |
| 4:17805583- | | | | | | | | | |
| 17805688:- | | | | | | | | | |
| ENST00000 | — | Detected | — | — | — | — | — | — | — |
| 590105.1_1_ | | | | | | | | | |
| 17:4297178 | | | | | | | | | |
| 0- | | | | | | | | | |
| 42971870:- | | | | | | | | | |
| ENST00000 | — | Detected | — | — | — | — | — | — | — |
| 462917.1_1_ | | | | | | | | | |
| 17:3984712 | | | | | | | | | |
| 0- | | | | | | | | | |
| 39847177:+ | | | | | | | | | |
| ENST00000 | — | Detected | — | — | — | — | — | — | — |
| 560763.5_2_ | | | | | | | | | |
| 14:1038005 | | | | | | | | | |
| 97- | | | | | | | | | |
| 103802258: | | | | | | | | | |
| + | | | | | | | | | |
| ENST00000 | — | Detected | — | — | — | — | — | — | — |
| 427574.6_1_ | | | | | | | | | |
| 11:7615614 | | | | | | | | | |
| 9- | | | | | | | | | |
| 76164398:+ | | | | | | | | | |
| ENST00000 | — | Detected | — | — | — | — | — | — | — |
| 269214.9_1_ | | | | | | | | | |
| 18:1915518 | | | | | | | | | |
| 3- | | | | | | | | | |
| 19155270:- | | | | | | | | | |
| ENST00000 | — | Detected | — | — | — | — | — | — | — |
| 369183.8_1_ | | | | | | | | | |
| 10:1200957 | | | | | | | | | |
| 85- | | | | | | | | | |
| 120095896:- | | | | | | | | | |
| ENST00000 | — | Detected | — | — | — | — | — | — | — |
| 327470.4_1_ | | | | | | | | | |
| 11:2264711 | | | | | | | | | |
| 5- | | | | | | | | | |
| 22647214:- | | | | | | | | | |
| ENST00000 | — | Detected | — | — | — | — | — | — | — |
| 424349.1_1_ | | | | | | | | | |
| 3:14987427- | | | | | | | | | |
| 14987592:- | | | | | | | | | |
| 1 | | | | | | | | | |
| ENST00000 | — | Detected | — | — | — | — | — | — | — |
| 454129.5_1_ | | | | | | | | | |
| 6:30260032- | | | | | | | | | |
| 30260266:- | | | | | | | | | |
| ENST00000 | — | Detected | — | — | — | — | — | — | — |
| 552912.5_1_ | | | | | | | | | |
| 12:1106183 | | | | | | | | | |
| 29- | | | | | | | | | |
| 110628779: | | | | | | | | | |
| + | | | | | | | | | |
| ENST00000 | — | Detected | — | — | — | — | — | — | — |
| 488494.5_1_ | | | | | | | | | |
| 10:3322143 | | | | | | | | | |
| 7- | | | | | | | | | |
| 33221518:- | | | | | | | | | |
| ENST00000 | — | Detected | — | — | — | — | — | — | — |
| 355300.6_1_ | | | | | | | | | |
| 15:7790824 | | | | | | | | | |
| 1- | | | | | | | | | |
| 77924773:- | | | | | | | | | |
| ENST00000 | — | Detected | — | — | — | — | — | — | — |
| 282892.3_1_ | | | | | | | | | |
| 12:2717935 | | | | | | | | | |
| 9- | | | | | | | | | |
| 27180306:+ | | | | | | | | | |
| ENST00000 | — | Detected | — | — | — | — | — | — | — |
| 603246.1_1_ | | | | | | | | | |
| 02 | | | | | | | | | |
| 5:99389051- | | | | | | | | | |
| 99389243:- | | | | | | | | | |
| ENST00000 | — | Detected | — | — | — | — | — | — | — |
| 318524.6_1_ | | | | | | | | | |
| 9:33294912- | | | | | | | | | |
| 33295365:+ | | | | | | | | | |
| ENST00000 | — | Detected | — | — | — | — | — | — | — |
| 337451.7_1_ | | | | | | | | | |
| 15:2303420 | | | | | | | | | |
| 9- | | | | | | | | | |
| 23034353:- | | | | | | | | | |
| ENST00000 | — | Detected | — | — | — | — | — | — | — |
| 423427.1_2_ | | | | | | | | | |
| 3:17332214 | | | | | | | | | |
| 8- | | | | | | | | | |
| 173322187: | | | | | | | | | |
| + | | | | | | | | | |
| ENST00000 | — | Detected | — | — | — | — | — | — | — |
| 332995.11_1_ | | | | | | | | | |
| 6:1328371 | | | | | | | | | |
| 1- | | | | | | | | | |
| 13286435:+ | | | | | | | | | |
| ENST00000 | — | Detected | — | — | — | — | — | — | — |
| 613674.4_1_ | | | | | | | | | |
| 5:10246506 | | | | | | | | | |
| 4- | | | | | | | | | |
| 102465157: | | | | | | | | | |
| + | | | | | | | | | |
| ENST00000 | — | Detected | — | — | — | — | — | — | — |
| 345306.10_2_ | | | | | | | | | |
| 5:6866568 | | | | | | | | | |
| 5- | | | | | | | | | |
| 68667980:+ | | | | | | | | | |
| ENST00000 | — | Detected | — | — | — | — | — | — | — |
| 471172.1_1_ | | | | | | | | | |
| 10:1125908 | | | | | | | | | |
| 20- | | | | | | | | | |
| 112591150: | | | | | | | | | |
| + | | | | | | | | | |
| ENST00000 | — | Detected | — | — | — | — | — | — | — |
| 530759.1_1_ | | | | | | | | | |
| 11:7615533 | | | | | | | | | |
| 8- | | | | | | | | | |
| 76155506:- | | | | | | | | | |
| ENST00000 | — | Detected | — | — | — | — | — | — | — |
| 640129.1_1_ | | | | | | | | | |
| 12:9723336- | | | | | | | | | |
| 9723429:+ | | | | | | | | | |
| 1.1 | | | | | | | | | |
| ENST00000 | — | Detected | — | — | — | — | — | — | — |
| 639134.1_1_ | | | | | | | | | |
| 5:18049650- | | | | | | | | | |
| 18049725:+ | | | | | | | | | |
| ENST00000 | — | Detected | — | — | — | — | — | — | — |
| 532695.5_1_ | | | | | | | | | |
| 12:1230050 | | | | | | | | | |
| 70- | | | | | | | | | |
| 123006782:- | | | | | | | | | |
| ENST00000 | — | Detected | — | — | — | — | — | — | — |
| 383018.7_1_ | | | | | | | | | |
| 16:103936- | | | | | | | | | |
| 105489:+ | | | | | | | | | |
| ENST00000 | — | Detected | — | — | — | — | — | — | — |
| 397942.3_1_ | | | | | | | | | |
| 18:6799204 | | | | | | | | | |
| 9- | | | | | | | | | |
| 67992124:+ | | | | | | | | | |
| ENST00000 | — | Detected | — | — | — | — | — | — | — |
| 532995.5_1_ | | | | | | | | | |
| 11:8542998 | | | | | | | | | |
| 9- | | | | | | | | | |
| 85430118:- | | | | | | | | | |
| ENST00000 | — | Detected | — | — | — | — | — | — | — |
| 383746.7_2_ | | | | | | | | | |
| 3:44396236- | | | | | | | | | |
| 44396287:+ | | | | | | | | | |
| ENST00000 | — | Detected | — | — | — | — | — | — | — |
| 196169.7_2_ | | | | | | | | | |
| 13:6097111 | | | | | | | | | |
| 6- | | | | | | | | | |
| 61013880:+ | | | | | | | | | |
| ENST00000 | — | Detected | — | — | — | — | — | — | — |
| 397640.5_1_ | | | | | | | | | |
| 17:7397533 | | | | | | | | | |
| 4- | | | | | | | | | |
| 73975502:+ | | | | | | | | | |
| ENST00000 | — | Detected | — | — | — | — | — | — | — |
| 305135.9_1_ | | | | | | | | | |
| 3:18343294 | | | | | | | | | |
| 2- | | | | | | | | | |
| 183435501: | | | | | | | | | |
| + | | | | | | | | | |
| ENST00000 | — | Detected | — | — | — | — | — | — | — |
| 216268.5_1_ | | | | | | | | | |
| 22:5027698 | | | | | | | | | |
| 4- | | | | | | | | | |
| 50277221:+ | | | | | | | | | |
| ENST00000 | — | Detected | — | — | — | — | — | — | — |
| 297857.3_1_ | | | | | | | | | |
| 8:12426833 | | | | | | | | | |
| 9- | | | | | | | | | |
| 124279533:- | | | | | | | | | |
| ENST00000 | — | Detected | — | — | — | — | — | — | — |
| 456324.5_1_ | | | | | | | | | |
| 19:3672691 | | | | | | | | | |
| 1- | | | | | | | | | |
| 36727052:+ | | | | | | | | | |
| TCONS_00 | — | Detected | — | — | — | — | — | — | — |
| 011153_6:4 | | | | | | | | | |
| 611322- | | | | | | | | | |
| 4611508:+ | | | | | | | | | |
| T196935_2: | — | Detected | — | — | — | — | — | — | — |
| 113299507- | | | | | | | | | |
| 113299648:- | | | | | | | | | |
| ENST00000 | — | Detected | — | — | — | — | — | — | — |
| 405489.7_2_ | | | | | | | | | |
| 2:27435269- | | | | | | | | | |
| 27435350:+ | | | | | | | | | |
| ENST00000 | — | — | Detected | — | — | — | — | — | — |
| 285093.14_1_ | | | | | | | | | |
| 18:473401 | | | | | | | | | |
| 24- | | | | | | | | | |
| 47340193:- | | | | | | | | | |
| ENST00000 | — | — | Detected | — | — | — | — | — | — |
| 597227.5_1_ | | | | | | | | | |
| 19:5043251 | | | | | | | | | |
| 4- | | | | | | | | | |
| 50434160:+ | | | | | | | | | |
| ENST00000 | — | — | Detected | — | — | — | — | — | — |
| 613993.1_2_ | | | | | | | | | |
| 6:10676400 | | | | | | | | | |
| 1- | | | | | | | | | |
| 106764076:- | | | | | | | | | |
| ENST00000 | — | — | Detected | — | — | — | — | — | — |
| 479665.5_2_ | | | | | | | | | |
| 3:28390190- | | | | | | | | | |
| 28390412:- | | | | | | | | | |
| ENST00000 | — | — | Detected | — | — | — | — | — | — |
| 399075.6_1_ | | | | | | | | | |
| 4:12021988 | | | | | | | | | |
| 6- | | | | | | | | | |
| 120221493:- | | | | | | | | | |
| ENST00000 | — | — | Detected | — | — | — | — | — | — |
| 461631.1_1_ | | | | | | | | | |
| 10:1052105 | | | | | | | | | |
| 16- | | | | | | | | | |
| 105210915:- | | | | | | | | | |
| ENST00000 | — | — | Detected | — | — | — | — | — | — |
| 482059.6_1_ | | | | | | | | | |
| 7:80267993- | | | | | | | | | |
| 80269165:+ | | | | | | | | | |
| ENST00000 | — | — | Detected | — | — | — | — | — | — |
| 366769.7_1_ | | | | | | | | | |
| 1:22750557 | | | | | | | | | |
| 0- | | | | | | | | | |
| 227505621:- | | | | | | | | | |
| ENST00000 | — | — | Detected | — | — | — | — | — | — |
| 567109.5_1_ | | | | | | | | | |
| 16:8266058 | | | | | | | | | |
| 3- | | | | | | | | | |
| 82660685:+ | | | | | | | | | |
| ENST00000 | — | — | Detected | — | — | — | — | — | — |
| 267843.8_1_ | | | | | | | | | |
| 15:4971645 | | | | | | | | | |
| 9- | | | | | | | | | |
| 49716492:+ | | | | | | | | | |
| ENST00000 | — | — | Detected | — | — | — | — | — | — |
| 303924.4_1_ | | | | | | | | | |
| 8:12265311 | | | | | | | | | |
| 1- | | | | | | | | | |
| 122653336:- | | | | | | | | | |
| ENST00000 | — | — | Detected | — | — | — | — | — | — |
| 440699.1_1_ | | | | | | | | | |
| 13:2152363 | | | | | | | | | |
| 0- | | | | | | | | | |
| 21523738:- | | | | | | | | | |
| ENST00000 | — | — | Detected | — | — | — | — | — | — |
| 396253.7_1_ | | | | | | | | | |
| 11:1833356 | | | | | | | | | |
| 1- | | | | | | | | | |
| 18343686:- | | | | | | | | | |
| ENST00000 | — | — | Detected | — | — | — | — | — | — |
| 233813.4_1_ | | | | | | | | | |
| 2:21755950 | | | | | | | | | |
| 7- | | | | | | | | | |
| 217559828:- | | | | | | | | | |
| ENST00000 | — | — | Detected | — | — | — | — | — | — |
| 597017.5_1_ | | | | | | | | | |
| 19:4889292 | | | | | | | | | |
| 4- | | | | | | | | | |
| 48893852:- | | | | | | | | | |
| ENST00000 | — | — | Detected | — | — | — | — | — | — |
| 230538.11_1_ | | | | | | | | | |
| 6:1125756 | | | | | | | | | |
| 72- | | | | | | | | | |
| 112575810:- | | | | | | | | | |
| ENST00000 | — | — | Detected | — | — | — | — | — | — |
| 435469.2_1_ | | | | | | | | | |
| 17:6609917 | | | | | | | | | |
| 1- | | | | | | | | | |
| 66110645:+ | | | | | | | | | |
| ENST00000 | — | — | Detected | — | — | — | — | — | — |
| 470907.6_1_ | | | | | | | | | |
| 2:74763585- | | | | | | | | | |
| 74776752:- | | | | | | | | | |
| ENST00000 | — | — | Detected | — | — | — | — | — | — |
| 564288.5_2_ | | | | | | | | | |
| 1:39670537- | | | | | | | | | |
| 39670564:+ | | | | | | | | | |
| ENST00000 | — | — | Detected | — | — | — | — | — | — |
| 347364.7_1_ | | | | | | | | | |
| 20:1040126 | | | | | | | | | |
| 9- | | | | | | | | | |
| 10414870:- | | | | | | | | | |
| ENST00000 | — | — | Detected | — | — | — | — | — | — |
| 219070.8_1_ | | | | | | | | | |
| 16:5551329 | | | | | | | | | |
| 6- | | | | | | | | | |
| 55513404:+ | | | | | | | | | |
| ENST00000 | — | — | Detected | — | — | — | — | — | — |
| 527046.5_1_ | | | | | | | | | |
| 14:2468639 | | | | | | | | | |
| 1- | | | | | | | | | |
| 24686427:- | | | | | | | | | |
| ENST00000 | — | — | Detected | — | — | — | — | — | — |
| 557673.5_3_ | | | | | | | | | |
| 14:7559088 | | | | | | | | | |
| 9- | | | | | | | | | |
| 75593687:- | | | | | | | | | |
| ENST00000 | — | — | Detected | — | — | — | — | — | — |
| 578207.5_1_ | | | | | | | | | |
| 1:14524884 | | | | | | | | | |
| 8- | | | | | | | | | |
| 145273378: | | | | | | | | | |
| + | | | | | | | | | |
| ENST00000 | — | — | Detected | — | — | — | — | — | — |
| 526593.1_1_ | | | | | | | | | |
| 11:6837705 | | | | | | | | | |
| 5- | | | | | | | | | |
| 68380582:+ | | | | | | | | | |
| ENST00000 | — | — | Detected | — | — | — | — | — | — |
| 558634.5_1_ | | | | | | | | | |
| 14:2461596 | | | | | | | | | |
| 1- | | | | | | | | | |
| 24617291:+ | | | | | | | | | |
| ENST00000 | — | — | Detected | — | — | — | — | — | — |
| 372209.3_1_ | | | | | | | | | |
| 1:45241798- | | | | | | | | | |
| 45243366:+ | | | | | | | | | |
| ENST00000 | — | — | Detected | — | — | — | — | — | — |
| 429490.5_1_ | | | | | | | | | |
| 10:5219328 | | | | | | | | | |
| 0- | | | | | | | | | |
| 52220465:- | | | | | | | | | |
| ENST00000 | — | — | Detected | — | — | — | — | — | — |
| 222345.10_1_ | | | | | | | | | |
| 19:385719 | | | | | | | | | |
| 27- | | | | | | | | | |
| 38572134:+ | | | | | | | | | |
| ENST00000 | — | — | Detected | — | — | — | — | — | — |
| 367097.7_1_ | | | | | | | | | |
| 6:15873486 | | | | | | | | | |
| 7- | | | | | | | | | |
| 158734906: | | | | | | | | | |
| + | | | | | | | | | |
| ENST00000 | — | — | Detected | — | — | — | — | — | — |
| 354940.10_1_ | | | | | | | | | |
| 12:104680 | | | | | | | | | |
| 894- | | | | | | | | | |
| 104681023: | | | | | | | | | |
| + | | | | | | | | | |
| ENST00000 | — | — | Detected | — | — | — | — | — | — |
| 332211.10_2_ | | | | | | | | | |
| 10:769705 | | | | | | | | | |
| 48- | | | | | | | | | |
| 76973790:+ | | | | | | | | | |
| ENST00000 | — | — | Detected | — | — | — | — | — | — |
| 311417.6_1_ | | | | | | | | | |
| 3:17878553 | | | | | | | | | |
| 6- | | | | | | | | | |
| 178789480:- | | | | | | | | | |
| ENST00000 | — | — | Detected | — | — | — | — | — | — |
| 299237.2_1_ | | | | | | | | | |
| 16:5803356 | | | | | | | | | |
| 8- | | | | | | | | | |
| 58033697:- | | | | | | | | | |
| ENST00000 | — | — | Detected | — | — | — | — | — | — |
| 584588.5_1_ | | | | | | | | | |
| 17:3802778 | | | | | | | | | |
| 2- | | | | | | | | | |
| 38031519:+ | | | | | | | | | |
| ENST00000 | — | — | Detected | — | — | — | — | — | — |
| 370096.7_1_ | | | | | | | | | |
| 1:10357400 | | | | | | | | | |
| 0- | | | | | | | | | |
| 103574030:- | | | | | | | | | |
| ENST00000 | — | — | Detected | — | — | — | — | — | — |
| 367590.8_1_ | | | | | | | | | |
| 1:18060119 | | | | | | | | | |
| 2- | | | | | | | | | |
| 180601402: | | | | | | | | | |
| + | | | | | | | | | |
| ENST00000 | Detected | — | Detected | — | — | — | — | — | — |
| 379251.7_1_ | | | | | | | | | |
| X:23801394- | | | | | | | | | |
| 2380148 1 :+ | | | | | | | | | |
| ENST00000 | Detected | — | Detected | — | — | — | — | — | — |
| 419821.6_1_ | | | | | | | | | |
| 12:1186934 | | | | | | | | | |
| 56- | | | | | | | | | |
| 118704503:- | | | | | | | | | |
| ENST00000 | — | Detected | Detected | — | — | — | — | — | — |
| 532383.5_1_ | | | | | | | | | |
| 11:1082507 | | | | | | | | | |
| 0- | | | | | | | | | |
| 10825157:- | | | | | | | | | |
| ENST00000 | — | Detected | Detected | — | — | — | — | — | — |
| 571129.5_1_ | | | | | | | | | |
| 17:7144048- | | | | | | | | | |
| 7144743:- | | | | | | | | | |
| ENST00000 | — | — | — | Detected | — | — | — | — | — |
| 391832.7_1_ | | | | | | | | | |
| 19:5038039 | | | | | | | | | |
| 4- | | | | | | | | | |
| 50380442:- | | | | | | | | | |
| ENST00000 | — | — | — | Detected | — | — | — | — | — |
| 383038.3_22: | | | | | | | | | |
| 16189326- | | | | | | | | | |
| 16190688:- | | | | | | | | | |
| ENST00000 | — | — | — | Detected | — | — | — | — | — |
| 375533.5_1_ | | | | | | | | | |
| 10:2897027 | | | | | | | | | |
| 9- | | | | | | | | | |
| 28970974:+ | | | | | | | | | |
| ENST00000 | — | — | — | Detected | — | — | — | — | — |
| 611077.4_1_ | | | | | | | | | |
| 19:4532319 | | | | | | | | | |
| 6- | | | | | | | | | |
| 45323622:+ | | | | | | | | | |
| ENST00000 | — | — | — | Detected | — | — | — | — | — |
| 456319.5_2_ | | | | | | | | | |
| 2:47397898- | | | | | | | | | |
| 47403621:- | | | | | | | | | |
| ENST00000 | — | — | — | Detected | — | — | — | — | — |
| 487264.5_1_ | | | | | | | | | |
| 19:5908684 | | | | | | | | | |
| 1- | | | | | | | | | |
| 59086880:+ | | | | | | | | | |
| ENST00000 | — | — | — | Detected | — | — | — | — | — |
| 305850.9_1_ | | | | | | | | | |
| 16:8104085 | | | | | | | | | |
| 7- | | | | | | | | | |
| 81045584:+ | | | | | | | | | |
| ENST00000 | — | — | — | Detected | — | — | — | — | — |
| 390664.2_1_ | | | | | | | | | |
| 16:7551577 | | | | | | | | | |
| 4- | | | | | | | | | |
| 75524658:- | | | | | | | | | |
| ENST00000 | — | — | — | Detected | — | — | — | — | — |
| 361654.8_3_ | | | | | | | | | |
| 12:1228650 | | | | | | | | | |
| 79- | | | | | | | | | |
| 122907104:- | | | | | | | | | |
| ENST00000 | — | — | — | Detected | — | — | — | — | — |
| 395879.5_2_ | | | | | | | | | |
| 7:43688212- | | | | | | | | | |
| 43688257:- | | | | | | | | | |
| ENST00000 | — | — | — | Detected | — | — | — | — | — |
| 310396.9_1_ | | | | | | | | | |
| 7:12062892 | | | | | | | | | |
| 5- | | | | | | | | | |
| 120629484: | | | | | | | | | |
| + | | | | | | | | | |
| ENST00000 | — | — | — | Detected | — | — | — | — | — |
| 526180.5_2_ | | | | | | | | | |
| 11:1117825 | | | | | | | | | |
| 75- | | | | | | | | | |
| 111782635:- | | | | | | | | | |
| ENST00000 | — | — | — | Detected | — | — | — | — | — |
| 302823.7_1_ | | | | | | | | | |
| 2:20473260 | | | | | | | | | |
| 6- | | | | | | | | | |
| 204732705: | | | | | | | | | |
| + | | | | | | | | | |
| ENST00000 | — | — | — | Detected | — | — | — | — | — |
| 532463.5_1_ | | | | | | | | | |
| 11:5752952 | | | | | | | | | |
| 9- | | | | | | | | | |
| 57561533:+ | | | | | | | | | |
| ENST00000 | — | — | — | Detected | — | — | — | — | — |
| 257215.9_1_ | | | | | | | | | |
| 11:6144792 | | | | | | | | | |
| 7- | | | | | | | | | |
| 61487603:+ | | | | | | | | | |
| ENST00000 | — | — | — | Detected | — | — | — | — | — |
| 504961.1_1_ | | | | | | | | | |
| 5:11231260 | | | | | | | | | |
| 1- | | | | | | | | | |
| 112319690: | | | | | | | | | |
| + | | | | | | | | | |
| ENST00000 | — | — | — | Detected | — | — | — | — | — |
| 263579.4_1_ | | | | | | | | | |
| 11:1261764 | | | | | | | | | |
| 68- | | | | | | | | | |
| 126176510: | | | | | | | | | |
| + | | | | | | | | | |
| ENST00000 | — | — | — | Detected | — | — | — | — | — |
| 377087.3_1_ | | | | | | | | | |
| 22:3106000 | | | | | | | | | |
| 9- | | | | | | | | | |
| 31063612:- | | | | | | | | | |
| ENST00000 | — | — | — | Detected | — | — | — | — | — |
| 405015.7_1_ | | | | | | | | | |
| 2:62934186- | | | | | | | | | |
| 62934213:+ | | | | | | | | | |
| ENST00000 | — | — | — | Detected | — | — | — | — | — |
| 487151.1_1_ | | | | | | | | | |
| 3:40352488- | | | | | | | | | |
| 40352533:+ | | | | | | | | | |
| ENST00000 | — | — | — | Detected | — | — | — | — | — |
| 469257.1_1_ | | | | | | | | | |
| 17:3984527 | | | | | | | | | |
| 6- | | | | | | | | | |
| 39846098:+ | | | | | | | | | |
| ENST00000 | — | — | — | Detected | — | — | — | — | — |
| 423854.6_1_ | | | | | | | | | |
| 18:3372281 | | | | | | | | | |
| 0- | | | | | | | | | |
| 33722882:+ | | | | | | | | | |
| ENST00000 | — | — | — | Detected | — | — | — | — | — |
| 360031.6_2_ | | | | | | | | | |
| 20:2517643 | | | | | | | | | |
| 9- | | | | | | | | | |
| 25187192:+ | | | | | | | | | |
| ENST00000 | — | — | — | Detected | — | — | — | — | — |
| 398334.5_2_ | | | | | | | | | |
| X:14887065- | | | | | | | | | |
| 14891178:- | | | | | | | | | |
| ENST00000 | — | — | — | Detected | — | — | — | — | — |
| 534938.6_2_ | | | | | | | | | |
| 14:7517989 | | | | | | | | | |
| 4- | | | | | | | | | |
| 75180224:+ | | | | | | | | | |
| ENST00000 | — | — | — | Detected | — | — | — | — | — |
| 598319.5_1_ | | | | | | | | | |
| 19:5002790 | | | | | | | | | |
| 1- | | | | | | | | | |
| 50027952:+ | | | | | | | | | |
| ENST00000 | — | — | — | Detected | — | — | — | — | — |
| 612683.1_1_ | | | | | | | | | |
| 1:15527985 | | | | | | | | | |
| 9- | | | | | | | | | |
| 155279940: | | | | | | | | | |
| + | | | | | | | | | |
| T266625_4: | — | — | — | Detected | — | — | — | — | — |
| 76104055- | | | | | | | | | |
| 76105674:- | | | | | | | | | |
| ENST00000 | — | — | — | Detected | — | — | — | — | — |
| 336898.7_1_ | | | | | | | | | |
| 11:7523691 | | | | | | | | | |
| 3- | | | | | | | | | |
| 75236943:- | | | | | | | | | |
| ENST00000 | — | — | — | Detected | — | — | — | — | — |
| 299314.11_1_ | | | | | | | | | |
| 12:102183 | | | | | | | | | |
| 791- | | | | | | | | | |
| 102224401:- | | | | | | | | | |
| ENST00000 | — | — | — | Detected | — | — | — | — | — |
| 446054.1_1_ | | | | | | | | | |
| 7:84506012- | | | | | | | | | |
| 84506255:+ | | | | | | | | | |
| ENST00000 | — | — | — | Detected | — | — | — | — | — |
| 356956.5_2_ | | | | | | | | | |
| 6:16052742 | | | | | | | | | |
| 9- | | | | | | | | | |
| 160527663: | | | | | | | | | |
| + | | | | | | | | | |
| ENST00000 | — | — | — | Detected | — | — | — | — | — |
| 374647.9_1_ | | | | | | | | | |
| 9:11168971 | | | | | | | | | |
| 4- | | | | | | | | | |
| 111692194:- | | | | | | | | | |
| ENST00000 | — | — | — | Detected | — | — | — | — | — |
| 374864.8_3_ | | | | | | | | | |
| 20:3295720 | | | | | | | | | |
| 4- | | | | | | | | | |
| 32957252:+ | | | | | | | | | |
| ENST00000 | — | — | — | Detected | — | — | — | — | — |
| 200639.8_2_ | | | | | | | | | |
| X:11958293 | | | | | | | | | |
| 8- | | | | | | | | | |
| 119589226:- | | | | | | | | | |
| ENST00000 | — | — | — | Detected | — | — | — | — | — |
| 394434.6_1_ | | | | | | | | | |
| 5:14556216 | | | | | | | | | |
| 7- | | | | | | | | | |
| 145562200:- | | | | | | | | | |
| ENST00000 | — | — | — | Detected | — | — | — | — | — |
| 412609.5_1_ | | | | | | | | | |
| 19:4017005 | | | | | | | | | |
| 9- | | | | | | | | | |
| 40174276:+ | | | | | | | | | |
| ENST00000 | — | — | — | Detected | — | — | — | — | — |
| 589878.5_1_ | | | | | | | | | |
| 19:3466342 | | | | | | | | | |
| 9- | | | | | | | | | |
| 34663453:+ | | | | | | | | | |
| ENST00000 | — | — | — | Detected | — | — | — | — | — |
| 395491.6_1_ | | | | | | | | | |
| 17:2118813 | | | | | | | | | |
| 5- | | | | | | | | | |
| 21188198:+ | | | | | | | | | |
| ENST00000 | — | — | — | Detected | — | — | — | — | — |
| 432910.5_1_ | | | | | | | | | |
| 20:5889587 | | | | | | | | | |
| 4- | | | | | | | | | |
| 58896255:+ | | | | | | | | | |
| ENST00000 | — | — | — | Detected | — | — | — | — | — |
| 568194.5_1_ | | | | | | | | | |
| 16:2255935- | | | | | | | | | |
| 2255977:+ | | | | | | | | | |
| ENST00000 | — | — | — | Detected | — | — | — | — | — |
| 428879.5_1_ | | | | | | | | | |
| 2:15043608 | | | | | | | | | |
| 1- | | | | | | | | | |
| 150438721:- | | | | | | | | | |
| ENST00000 | — | — | — | Detected | — | — | — | — | — |
| 281513.9_1_ | | | | | | | | | |
| 2:15694206- | | | | | | | | | |
| 15694242:- | | | | | | | | | |
| ENST00000 | — | — | — | Detected | — | — | — | — | — |
| 337451.7_1_ | | | | | | | | | |
| 15:2301447 | | | | | | | | | |
| 1- | | | | | | | | | |
| 23019844:- | | | | | | | | | |
| ENST00000 | — | — | — | Detected | — | — | — | — | — |
| 485741.6_1_ | | | | | | | | | |
| 7:72424097- | | | | | | | | | |
| 72424166:- | | | | | | | | | |
| ENST00000 | — | — | — | Detected | — | — | — | — | — |
| 614443.1_1_ | | | | | | | | | |
| 17:3489093 | | | | | | | | | |
| 8- | | | | | | | | | |
| 34891122:+ | | | | | | | | | |
| ENST00000 | — | — | — | Detected | — | — | — | — | — |
| 527403.6_2_ | | | | | | | | | |
| 11:6830522 | | | | | | | | | |
| 6- | | | | | | | | | |
| 68305268:+ | | | | | | | | | |
| ENST00000 | — | — | — | Detected | — | — | — | — | — |
| 477626.5_1_ | | | | | | | | | |
| 14:4556443 | | | | | | | | | |
| 1- | | | | | | | | | |
| 45564512:+ | | | | | | | | | |
| ENST00000 | — | — | — | Detected | — | — | — | — | — |
| 280258.5_1_ | | | | | | | | | |
| 11:8651886 | | | | | | | | | |
| 3- | | | | | | | | | |
| 86519148:+ | | | | | | | | | |
| ENST00000 | — | — | — | Detected | — | — | — | — | — |
| 409433.2_1_ | | | | | | | | | |
| 17:5777749 | | | | | | | | | |
| 5- | | | | | | | | | |
| 57784778:- | | | | | | | | | |
| ENST00000 | — | — | — | Detected | — | — | — | — | — |
| 520191.5_1_ | | | | | | | | | |
| 8:30402888- | | | | | | | | | |
| 30402969:+ | | | | | | | | | |
| ENST00000 | — | — | — | Detected | — | — | — | — | — |
| 468480.5_1_ | | | | | | | | | |
| 13:2679538 | | | | | | | | | |
| 8- | | | | | | | | | |
| 26795430:- | | | | | | | | | |
| ENST00000 | — | — | — | Detected | — | — | — | — | — |
| 567508.1_1_ | | | | | | | | | |
| 1:40723236- | | | | | | | | | |
| 40723284:- | | | | | | | | | |
| ENST00000 | — | — | — | Detected | — | — | — | — | — |
| 563764.2_2_ | | | | | | | | | |
| 16:7666941 | | | | | | | | | |
| 3- | | | | | | | | | |
| 76669506:+ | | | | | | | | | |
| ENST00000 | — | — | — | Detected | — | — | — | — | — |
| 415279.1_1_ | | | | | | | | | |
| 1:10643493 | | | | | | | | | |
| 0- | | | | | | | | | |
| 106435587: | | | | | | | | | |
| + | | | | | | | | | |
| ENST00000 | — | — | — | Detected | — | — | — | — | — |
| 531908.5_1_ | | | | | | | | | |
| 11:1709712 | | | | | | | | | |
| 2- | | | | | | | | | |
| 17097149:- | | | | | | | | | |
| ENST00000 | — | — | — | Detected | — | — | — | — | — |
| 312037.5_1_ | | | | | | | | | |
| 5:14982650 | | | | | | | | | |
| 6- | | | | | | | | | |
| 149829126:- | | | | | | | | | |
| ENST00000 | — | — | — | Detected | — | — | — | — | — |
| 271638.2_1_ | | | | | | | | | |
| 1:15200615 | | | | | | | | | |
| 8- | | | | | | | | | |
| 152006227:- | | | | | | | | | |
| ENST00000 | — | — | — | Detected | — | — | — | — | — |
| 336735.8_1_ | | | | | | | | | |
| 14:8197060 | | | | | | | | | |
| 2- | | | | | | | | | |
| 81972522:- | | | | | | | | | |
| ENST00000 | — | — | — | Detected | — | — | — | — | — |
| 428443.7_1_ | | | | | | | | | |
| 2:18001117 | | | | | | | | | |
| 0- | | | | | | | | | |
| 180014060:- | | | | | | | | | |
| ENST00000 | — | — | — | Detected | — | — | — | — | — |
| 630869.1_1_ | | | | | | | | | |
| 1:16821209 | | | | | | | | | |
| 6- | | | | | | | | | |
| 168212303: | | | | | | | | | |
| + | | | | | | | | | |
| ENST00000 | — | — | — | Detected | — | — | — | — | — |
| 415059.1_1_ | | | | | | | | | |
| 1:10895275 | | | | | | | | | |
| 2- | | | | | | | | | |
| 108963771:- | | | | | | | | | |
| ENST00000 | — | — | — | Detected | — | — | — | — | — |
| 484515.1_1_ | | | | | | | | | |
| 20:4158069- | | | | | | | | | |
| 4162462:+ | | | | | | | | | |
| ENST00000 | — | — | — | Detected | — | — | — | — | — |
| 020945.3_2_ | | | | | | | | | |
| 8:49832997- | | | | | | | | | |
| 49833910:- | | | | | | | | | |
| ENST00000 | — | — | — | Detected | — | — | — | — | — |
| 222990.7_1_ | | | | | | | | | |
| 7:2311567- | | | | | | | | | |
| 2317760:- | | | | | | | | | |
| ENST00000 | — | — | — | Detected | — | — | — | — | — |
| 488132.5_1_ | | | | | | | | | |
| 3:98434244- | | | | | | | | | |
| 98434552:- | | | | | | | | | |
| ENST00000 | — | — | — | Detected | — | — | — | — | — |
| 528107.5_2_ | | | | | | | | | |
| 11:3306171 | | | | | | | | | |
| 8- | | | | | | | | | |
| 33061820:+ | | | | | | | | | |
| ENST00000 | — | — | — | Detected | — | — | — | — | — |
| 528688.5_1_ | | | | | | | | | |
| 11:7900853 | | | | | | | | | |
| 5- | | | | | | | | | |
| 79150030:- | | | | | | | | | |
| ENST00000 | — | — | — | Detected | — | — | — | — | — |
| 552370.5_1_ | | | | | | | | | |
| 12:5014678 | | | | | | | | | |
| 4- | | | | | | | | | |
| 50149448:+ | | | | | | | | | |
| ENST00000 | — | — | — | Detected | — | — | — | — | — |
| 510089.5_1_ | | | | | | | | | |
| 5:87524264- | | | | | | | | | |
| 87564570:- | | | | | | | | | |
| ENST00000 | — | — | — | Detected | — | — | — | — | — |
| 367242.3_1_ | | | | | | | | | |
| 1:20297653 | | | | | | | | | |
| 1- | | | | | | | | | |
| 202976618: | | | | | | | | | |
| + | | | | | | | | | |
| ENST00000 | — | — | — | Detected | — | — | — | — | — |
| 398559.6_1_ | | | | | | | | | |
| 3:69101351- | | | | | | | | | |
| 69101438:- | | | | | | | | | |
| ENST00000 | — | — | — | Detected | — | — | — | — | — |
| 296861.2_1_ | | | | | | | | | |
| 6:47277434- | | | | | | | | | |
| 47277623:- | | | | | | | | | |
| ENST00000 | — | — | — | Detected | — | — | — | — | — |
| 469470.1_1_ | | | | | | | | | |
| 7:47440019- | | | | | | | | | |
| 47440405:- | | | | | | | | | |
| ENST00000 | — | — | — | Detected | — | — | — | — | — |
| 548460.2_1_ | | | | | | | | | |
| 7:14453251 | | | | | | | | | |
| 4- | | | | | | | | | |
| 144532616:- | | | | | | | | | |
| ENST00000 | — | — | — | Detected | — | — | — | — | — |
| 417475.5_1_ | | | | | | | | | |
| 7:10046502 | | | | | | | | | |
| 5- | | | | | | | | | |
| 100465781: | | | | | | | | | |
| + | | | | | | | | | |
| ENST00000 | — | — | — | Detected | — | — | — | — | — |
| 459764.1_1_ | | | | | | | | | |
| 10:1027475 | | | | | | | | | |
| 80- | | | | | | | | | |
| 102749466: | | | | | | | | | |
| + | | | | | | | | | |
| ENST00000 | — | — | — | Detected | — | — | — | — | — |
| 526950.1_1_ | | | | | | | | | |
| 12:1047051 | | | | | | | | | |
| 13- | | | | | | | | | |
| 104707050: | | | | | | | | | |
| + | | | | | | | | | |
| ENST00000 | — | — | — | Detected | — | — | — | — | — |
| 410062.8_1_ | | | | | | | | | |
| 2:18184534 | | | | | | | | | |
| 3- | | | | | | | | | |
| 181846756: | | | | | | | | | |
| + | | | | | | | | | |
| ENST00000 | — | — | — | Detected | — | — | — | — | — |
| 528337.1_1_ | | | | | | | | | |
| 10:2881333 | | | | | | | | | |
| 7- | | | | | | | | | |
| 28813382:- | | | | | | | | | |
| ENST00000 | — | — | — | Detected | — | — | — | — | — |
| 316059.6_1_ | | | | | | | | | |
| 11:5835234 | | | | | | | | | |
| 8- | | | | | | | | | |
| 58378460:+ | | | | | | | | | |
| ENST00000 | — | — | — | Detected | — | — | — | — | — |
| 265069.12_1_ | | | | | | | | | |
| 5:3242018 | | | | | | | | | |
| 5- | | | | | | | | | |
| 32444730:- | | | | | | | | | |
| ENST00000 | — | — | — | Detected | — | — | — | — | — |
| 347230.8_2_ | | | | | | | | | |
| 14:6828272 | | | | | | | | | |
| 2- | | | | | | | | | |
| 68283293:- | | | | | | | | | |
| ENST00000 | — | — | — | Detected | — | — | — | — | — |
| 274712.7_1_ | | | | | | | | | |
| 5:14008044 | | | | | | | | | |
| 0- | | | | | | | | | |
| 140083534: | | | | | | | | | |
| + | | | | | | | | | |
| ENST00000 | — | — | — | Detected | — | — | — | — | — |
| 395797.1_1_ | | | | | | | | | |
| 10:4414023 | | | | | | | | | |
| 9- | | | | | | | | | |
| 44144057:- | | | | | | | | | |
| ENST00000 | — | — | — | Detected | — | — | — | — | — |
| 410075.5_1_ | | | | | | | | | |
| 2:71576016- | | | | | | | | | |
| 71576058:+ | | | | | | | | | |
| TCONS_00 | — | — | — | Detected | — | — | — | — | — |
| 018434_10: | | | | | | | | | |
| 10826571- | | | | | | | | | |
| 10834395:- | | | | | | | | | |
| ENST00000 | — | — | — | Detected | — | — | — | — | — |
| 303577.6_1_ | | | | | | | | | |
| 2:70315071- | | | | | | | | | |
| 70315098:+ | | | | | | | | | |
| ENST00000 | — | — | — | Detected | — | — | — | — | — |
| 402538.7_1_ | | | | | | | | | |
| 2:33740127- | | | | | | | | | |
| 33740184:+ | | | | | | | | | |
| ENST00000 | — | — | — | Detected | — | — | — | — | — |
| 532865.5_2_ | | | | | | | | | |
| 6:26365529- | | | | | | | | | |
| 26368244:+ | | | | | | | | | |
| ENST00000 | — | — | — | Detected | — | — | — | — | — |
| 378119.8_1_ | | | | | | | | | |
| 6:24720562- | | | | | | | | | |
| 24720727:- | | | | | | | | | |
| ENST00000 | — | — | — | Detected | — | — | — | — | — |
| 245615.5_1_ | | | | | | | | | |
| 19:5469229 | | | | | | | | | |
| 4- | | | | | | | | | |
| 54692348:- | | | | | | | | | |
| ENST00000 | — | — | — | Detected | — | — | — | — | — |
| 339995.9_2_ | | | | | | | | | |
| 11:1082506 | | | | | | | | | |
| 7- | | | | | | | | | |
| 10825719:- | | | | | | | | | |
| ENST00000 | — | Detected | — | Detected | — | — | — | — | — |
| 539080.1_1_ | | | | | | | | | |
| 12:1228650 | | | | | | | | | |
| 79- | | | | | | | | | |
| 122884682:- | | | | | | | | | |
| ENST00000 | — | Detected | — | Detected | — | — | — | — | — |
| 393094.6_1_ | | | | | | | | | |
| 11:1078796 | | | | | | | | | |
| 97- | | | | | | | | | |
| 107879871: | | | | | | | | | |
| + | | | | | | | | | |
| ENST00000 | — | Detected | — | Detected | — | — | — | — | — |
| 446994.6_1_ | | | | | | | | | |
| 20:4598552 | | | | | | | | | |
| 7- | | | | | | | | | |
| 45985632:- | | | | | | | | | |
| ENST00000 | — | — | — | — | Detected | — | — | — | — |
| 372348.6_1_ | | | | | | | | | |
| 9:13372953 | | | | | | | | | |
| 7- | | | | | | | | | |
| 133729609: | | | | | | | | | |
| + | | | | | | | | | |
| ENST00000 | — | — | — | — | Detected | — | — | — | — |
| 368474.8_1_ | | | | | | | | | |
| 1:15457505 | | | | | | | | | |
| 8- | | | | | | | | | |
| 154580648:- | | | | | | | | | |
| ENST00000 | — | — | — | — | Detected | — | — | — | — |
| 283296.11_1_ | | | | | | | | | |
| 6:4688956 | | | | | | | | | |
| 1- | | | | | | | | | |
| 46889594:- | | | | | | | | | |
| ENST00000 | — | — | — | — | Detected | — | — | — | — |
| 551310.1_1_ | | | | | | | | | |
| 12:9004980 | | | | | | | | | |
| 5- | | | | | | | | | |
| 90103048:- | | | | | | | | | |
| ENST00000 | — | — | — | — | Detected | — | — | — | — |
| 529783.1_1_ | | | | | | | | | |
| 2:74375310- | | | | | | | | | |
| 74375499:+ | | | | | | | | | |
| ENST00000 | — | — | — | — | Detected | — | — | — | — |
| 418040.5_1_ | | | | | | | | | |
| 7:13433159 | | | | | | | | | |
| 0- | | | | | | | | | |
| 134346220: | | | | | | | | | |
| + | | | | | | | | | |
| ENST00000 | — | — | — | — | Detected | — | — | — | — |
| 395789.5_1_ | | | | | | | | | |
| 6:31802780- | | | | | | | | | |
| 31805136:+ | | | | | | | | | |
| ENST00000 | — | — | — | — | Detected | — | — | — | — |
| 511697.1_1_ | | | | | | | | | |
| 17:4691906 | | | | | | | | | |
| 9- | | | | | | | | | |
| 46919841:+ | | | | | | | | | |
| ENST00000 | — | — | — | — | Detected | — | — | — | — |
| 466741.5_1_ | | | | | | | | | |
| 1:11177235 | | | | | | | | | |
| 9- | | | | | | | | | |
| 111772476: | | | | | | | | | |
| + | | | | | | | | | |
| ENST00000 | — | — | — | — | Detected | — | — | — | — |
| 512287.1_1_ | | | | | | | | | |
| 5:80531836- | | | | | | | | | |
| 80531875:- | | | | | | | | | |
| ENST00000 | — | — | — | — | Detected | — | — | — | — |
| 353704.2_2_ | | | | | | | | | |
| 9:35732632- | | | | | | | | | |
| 35732818:+ | | | | | | | | | |
| ENST00000 | — | — | — | — | Detected | — | — | — | — |
| 406892.6_1_ | | | | | | | | | |
| 19:1209863 | | | | | | | | | |
| 5- | | | | | | | | | |
| 12112633:+ | | | | | | | | | |
| ENST00000 | — | — | — | — | Detected | — | — | — | — |
| 373095.5_1_ | | | | | | | | | |
| 9:13071044 | | | | | | | | | |
| 5- | | | | | | | | | |
| 130712742:- | | | | | | | | | |
| ENST00000 | — | — | — | — | Detected | — | — | — | — |
| 566920.5_3_ | | | | | | | | | |
| 16:6756396 | | | | | | | | | |
| 7- | | | | | | | | | |
| 67572402:+ | | | | | | | | | |
| ENST00000 | — | — | — | — | Detected | — | — | — | — |
| 373555.8_1_ | | | | | | | | | |
| 9:12770107 | | | | | | | | | |
| 3- | | | | | | | | | |
| 127701109:- | | | | | | | | | |
| ENST00000 | — | — | — | — | Detected | — | — | — | — |
| 545875.4_1_ | | | | | | | | | |
| 12:1333988 | | | | | | | | | |
| 59- | | | | | | | | | |
| 133405404:- | | | | | | | | | |
| ENST00000 | — | — | — | — | Detected | — | — | — | — |
| 527431.1_1_ | | | | | | | | | |
| 11:9406310- | | | | | | | | | |
| 9430044:+ | | | | | | | | | |
| ENST00000 | — | — | — | — | Detected | — | — | — | — |
| 443364.6_1_ | | | | | | | | | |
| 1:15993117 | | | | | | | | | |
| 0- | | | | | | | | | |
| 159935305: | | | | | | | | | |
| + | | | | | | | | | |
| ENST00000 | — | — | — | — | Detected | — | — | — | — |
| 306888.6_1_ | | | | | | | | | |
| 4:54364989- | | | | | | | | | |
| 54373573:- | | | | | | | | | |
| ENST00000 | — | — | — | — | Detected | — | — | — | — |
| 446660.5_1_ | | | | | | | | | |
| 2:55490938- | | | | | | | | | |
| 55494722:- | | | | | | | | | |
| ENST00000 | — | — | — | — | Detected | — | — | — | — |
| 616174.1_1_ | | | | | | | | | |
| 3:11344293 | | | | | | | | | |
| 9- | | | | | | | | | |
| 113464857:- | | | | | | | | | |
| ENST00000 | — | — | — | — | Detected | — | — | — | — |
| 478653.6_1_ | | | | | | | | | |
| 1:21780423- | | | | | | | | | |
| 21780552:+ | | | | | | | | | |
| ENST00000 | — | — | — | — | Detected | — | — | — | — |
| 393196.7_1_ | | | | | | | | | |
| 17:4923925 | | | | | | | | | |
| 4- | | | | | | | | | |
| 49239476:+ | | | | | | | | | |
| ENST00000 | — | — | — | — | Detected | — | — | — | — |
| 495842.5_1_ | | | | | | | | | |
| 3:10150426 | | | | | | | | | |
| 2- | | | | | | | | | |
| 101504385: | | | | | | | | | |
| + | | | | | | | | | |
| ENST00000 | — | — | — | — | Detected | — | — | — | — |
| 259467.8_1_ | | | | | | | | | |
| 9:12558546 | | | | | | | | | |
| 1- | | | | | | | | | |
| 125589017:- | | | | | | | | | |
| ENST00000 | — | — | — | — | Detected | — | — | — | — |
| 361189.6_1_ | | | | | | | | | |
| 5:10871728 | | | | | | | | | |
| 7- | | | | | | | | | |
| 108719181:- | | | | | | | | | |
| ENST00000 | — | — | — | — | Detected | — | — | — | — |
| 532157.5_1_ | | | | | | | | | |
| 8:42196130- | | | | | | | | | |
| 42202487:+ | | | | | | | | | |
| ENST00000 | — | — | — | — | Detected | — | — | — | — |
| 380842.4_1_ | | | | | | | | | |
| 13:2923659 | | | | | | | | | |
| 2- | | | | | | | | | |
| 29238659:+ | | | | | | | | | |
| ENST00000 | — | — | — | — | Detected | — | — | — | — |
| 546265.1_1_ | | | | | | | | | |
| 12:1127374 | | | | | | | | | |
| 1- | | | | | | | | | |
| 11324198:- | | | | | | | | | |
| ENST00000 | — | — | — | — | Detected | — | — | — | — |
| 433510.2_1_ | | | | | | | | | |
| 17:4671350 | | | | | | | | | |
| 3- | | | | | | | | | |
| 46713704:- | | | | | | | | | |
| ENST00000 | — | — | — | — | Detected | — | — | — | — |
| 496652.5_1_ | | | | | | | | | |
| 22:5122171 | | | | | | | | | |
| 8- | | | | | | | | | |
| 51227271:+ | | | | | | | | | |
| ENST00000 | — | — | — | — | Detected | — | — | — | — |
| 469911.1_1_ | | | | | | | | | |
| 3:18232869 | | | | | | | | | |
| 4- | | | | | | | | | |
| 182329198: | | | | | | | | | |
| + | | | | | | | | | |
| ENST00000 | — | — | — | — | Detected | — | — | — | — |
| 493224.5_1_ | | | | | | | | | |
| 1:15396412 | | | | | | | | | |
| 6- | | | | | | | | | |
| 153964580: | | | | | | | | | |
| + | | | | | | | | | |
| ENST00000 | — | — | — | — | Detected | — | — | — | — |
| 320270.3_1_ | | | | | | | | | |
| 8:67341274- | | | | | | | | | |
| 67341313:+ | | | | | | | | | |
| ENST00000 | — | — | — | — | Detected | — | — | — | — |
| 223641.4_1_ | | | | | | | | | |
| 9:10198462 | | | | | | | | | |
| 5- | | | | | | | | | |
| 101992644: | | | | | | | | | |
| + | | | | | | | | | |
| ENST00000 | — | — | — | — | Detected | — | — | — | — |
| 556605.5_1_ | | | | | | | | | |
| 14:6986541 | | | | | | | | | |
| 4- | | | | | | | | | |
| 69865501:+ | | | | | | | | | |
| ENST00000 | — | — | — | — | Detected | — | — | — | — |
| 414282.5_1_ | | | | | | | | | |
| 9:13962151 | | | | | | | | | |
| 9- | | | | | | | | | |
| 139622634:- | | | | | | | | | |
| ENST00000 | — | — | — | — | Detected | — | — | — | — |
| 407501.6_1_ | | | | | | | | | |
| 18:3449895- | | | | | | | | | |
| 3450350:+ | | | | | | | | | |
| ENST00000 | — | — | — | — | Detected | — | — | — | — |
| 218388.8_1_ | | | | | | | | | |
| X:47445059 | | | | | | | | | |
| -47446002:+ | | | | | | | | | |
| ENST00000 | — | — | — | — | Detected | — | — | — | — |
| 488206.5_1_ | | | | | | | | | |
| 1:62175075- | | | | | | | | | |
| 62189407:- | | | | | | | | | |
| ENST00000 | — | — | — | — | Detected | — | — | — | — |
| 453776.5_1_ | | | | | | | | | |
| 2:21915056 | | | | | | | | | |
| 1- | | | | | | | | | |
| 219150675:- | | | | | | | | | |
| ENST00000 | — | — | — | — | Detected | — | — | — | — |
| 448392.5_1_ | | | | | | | | | |
| 15:4256560 | | | | | | | | | |
| 1- | | | | | | | | | |
| 42565628:- | | | | | | | | | |
| ENST00000 | — | — | — | — | Detected | — | — | — | — |
| 473131.5_1_ | | | | | | | | | |
| 20:5924535- | | | | | | | | | |
| 5931132:- | | | | | | | | | |
| ENST00000 | — | — | — | — | Detected | — | — | — | — |
| 399976.6_1_ | | | | | | | | | |
| 21:3039707 | | | | | | | | | |
| 1- | | | | | | | | | |
| 30400211:+ | | | | | | | | | |
| ENST00000 | — | — | — | — | Detected | — | — | — | — |
| 638800.1_1_ | | | | | | | | | |
| 1:20376562 | | | | | | | | | |
| 4- | | | | | | | | | |
| 203770741: | | | | | | | | | |
| + | | | | | | | | | |
| ENST00000 | — | — | — | — | Detected | — | — | — | — |
| 572691.2_2_ | | | | | | | | | |
| 16:3179460- | | | | | | | | | |
| 3179646:- | | | | | | | | | |
| TCONS_00 | — | — | — | — | Detected | — | — | — | — |
| 019109_11: | | | | | | | | | |
| 110207337- | | | | | | | | | |
| 110252699: | | | | | | | | | |
| + | | | | | | | | | |
| T267797_4: | — | — | — | — | Detected | — | — | — | — |
| 88400064- | | | | | | | | | |
| 88403668:+ | | | | | | | | | |
| T350279_8: | — | — | — | — | Detected | — | — | — | — |
| 120845279- | | | | | | | | | |
| 120865310: | | | | | | | | | |
| + | | | | | | | | | |
| ENST00000 | — | — | — | — | Detected | — | — | — | — |
| 640069.1_1_ | | | | | | | | | |
| 6:13661088 | | | | | | | | | |
| 2- | | | | | | | | | |
| 136610939:- | | | | | | | | | |
| ENST00000 | — | — | — | — | Detected | — | — | — | — |
| 369535.4_1_ | | | | | | | | | |
| 1:11525015 | | | | | | | | | |
| 5- | | | | | | | | | |
| 115250203:- | | | | | | | | | |
| ENST00000 | — | — | — | — | Detected | — | — | — | — |
| 557046.1_1_ | | | | | | | | | |
| 14:6990896 | | | | | | | | | |
| 5- | | | | | | | | | |
| 69921585:+ | | | | | | | | | |
| ENST00000 | — | — | — | — | Detected | — | — | — | — |
| 447578.6_1_ | | | | | | | | | |
| 12:1109309 | | | | | | | | | |
| 33- | | | | | | | | | |
| 110930975:- | | | | | | | | | |
| ENST00000 | — | — | — | — | Detected | — | — | — | — |
| 294016.7_1_ | | | | | | | | | |
| 16:4165851- | | | | | | | | | |
| 4165998:- | | | | | | | | | |
| ENST00000 | — | — | — | — | Detected | — | — | — | — |
| 376007.8_1_ | | | | | | | | | |
| 6:31588511- | | | | | | | | | |
| 31590634:+ | | | | | | | | | |
| ENST00000 | — | — | — | — | Detected | — | — | — | — |
| 303372.6_1_ | | | | | | | | | |
| 12:1241556 | | | | | | | | | |
| 84- | | | | | | | | | |
| 124155726: | | | | | | | | | |
| + | | | | | | | | | |
| ENST00000 | — | — | — | — | Detected | — | — | — | — |
| 257663.3_1_ | | | | | | | | | |
| 7:77423607- | | | | | | | | | |
| 77427674:- | | | | | | | | | |
| ENST00000 | — | Detected | — | — | Detected | — | — | — | — |
| 399022.8_1_ | | | | | | | | | |
| 18:3361369 | | | | | | | | | |
| 5- | | | | | | | | | |
| 33613761:- | | | | | | | | | |
| T301592_6: | — | Detected | — | — | Detected | — | — | — | — |
| 32441186- | | | | | | | | | |
| 32441927:+ | | | | | | | | | |
| ENST00000 | — | Detected | — | — | Detected | — | — | — | — |
| 361762.3_2_ | | | | | | | | | |
| 4:30722246- | | | | | | | | | |
| 30722306:+ | | | | | | | | | |
| ENST00000 | — | — | Detected | — | Detected | — | — | — | — |
| 487209.5_1_ | | | | | | | | | |
| 1:43830638- | | | | | | | | | |
| 43833598:- | | | | | | | | | |
| ENST00000 | — | — | Detected | — | Detected | — | — | — | — |
| 521797.l_l_ | | | | | | | | | |
| 5:16288400 | | | | | | | | | |
| 4- | | | | | | | | | |
| 162884578:- | | | | | | | | | |
| ENST00000 | — | — | Detected | — | Detected | — | — | — | — |
| 544553.1_1_ | | | | | | | | | |
| 11:6401345 | | | | | | | | | |
| 3- | | | | | | | | | |
| 64013543:+ | | | | | | | | | |
| ENST00000 | — | Detected | Detected | — | Detected | — | — | — | — |
| 441189.3_1_ | | | | | | | | | |
| X:41200742- | | | | | | | | | |
| 41206988:+ | | | | | | | | | |
| ENST00000 | Detected | Detected | Detected | — | Detected | — | — | — | — |
| 292591.11_1_ | | | | | | | | | |
| 5:1792289 | | | | | | | | | |
| 08- | | | | | | | | | |
| 179233794:- | | | | | | | | | |
| ENST00000 | — | — | — | — | — | Detected | — | — | — |
| 477642.5_1_ | | | | | | | | | |
| 2:38525261- | | | | | | | | | |
| 38527422:- | | | | | | | | | |
| ENST00000 | — | — | — | — | — | Detected | — | — | — |
| 345523.8_1_ | | | | | | | | | |
| 19:5130258 | | | | | | | | | |
| 4- | | | | | | | | | |
| 51305485:- | | | | | | | | | |
| ENST00000 | — | — | — | — | — | Detected | — | — | — |
| 373504.10_1_ | | | | | | | | | |
| X:7290088 | | | | | | | | | |
| 8- | | | | | | | | | |
| 72901020:+ | | | | | | | | | |
| ENST00000 | — | — | — | — | — | Detected | — | — | — |
| 606994.1_1_ | | | | | | | | | |
| 5:32174612- | | | | | | | | | |
| 32175047:+ | | | | | | | | | |
| ENST00000 | — | — | — | — | — | Detected | — | — | — |
| 361951.4_1__ | | | | | | | | | |
| 1:17379435 | | | | | | | | | |
| 1- | | | | | | | | | |
| 173795834: | | | | | | | | | |
| + | | | | | | | | | |
| ENST00000 | — | — | — | — | — | Detected | — | — | — |
| 518345.1_1_ | | | | | | | | | |
| 8:10925407 | | | | | | | | | |
| 8- | | | | | | | | | |
| 109254135:- | | | | | | | | | |
| ENST00000 | — | — | — | — | — | Detected | — | — | — |
| 479810.6_1_ | | | | | | | | | |
| 21:4269493 | | | | | | | | | |
| 0- | | | | | | | | | |
| 42716440:+ | | | | | | | | | |
| ENST00000 | — | — | — | — | — | Detected | — | — | — |
| 409807.5_1_ | | | | | | | | | |
| 2:10601311 | | | | | | | | | |
| 3- | | | | | | | | | |
| 106013155:- | | | | | | | | | |
| ENST00000 | — | — | — | — | — | Detected | — | — | — |
| 496455.6_1_ | | | | | | | | | |
| 3:15561148 | | | | | | | | | |
| 7- | | | | | | | | | |
| 155621652: | | | | | | | | | |
| + | | | | | | | | | |
| ENST00000 | — | — | — | — | — | Detected | — | — | — |
| 265713.6_2_ | | | | | | | | | |
| 8:41906525- | | | | | | | | | |
| 41906624:- | | | | | | | | | |
| ENST00000 | — | — | — | — | — | Detected | — | — | — |
| 372724.5_1_ | | | | | | | | | |
| 10:7659850 | | | | | | | | | |
| 7- | | | | | | | | | |
| 76602417:+ | | | | | | | | | |
| ENST00000 | — | — | — | — | — | Detected | — | — | — |
| 372725.5_1_ | | | | | | | | | |
| 10:7660269 | | | | | | | | | |
| 7- | | | | | | | | | |
| 76602946:+ | | | | | | | | | |
| ENST00000 | — | — | — | — | — | Detected | — | — | — |
| 395392.6_2_ | | | | | | | | | |
| 15:6970978 | | | | | | | | | |
| 5- | | | | | | | | | |
| 69714354:+ | | | | | | | | | |
| ENST00000 | — | — | — | — | — | Detected | — | — | — |
| 640804.1_1_ | | | | | | | | | |
| 11:9351753 | | | | | | | | | |
| 0- | | | | | | | | | |
| 93517563:+ | | | | | | | | | |
| ENST00000 | — | — | — | — | — | Detected | — | — | — |
| 482361.1_1_ | | | | | | | | | |
| 6:41877193- | | | | | | | | | |
| 41888846:- | | | | | | | | | |
| ENST00000 | — | — | — | — | — | Detected | — | — | — |
| 334350.6_1_ | | | | | | | | | |
| 16:5669163 | | | | | | | | | |
| 0- | | | | | | | | | |
| 56693012:+ | | | | | | | | | |
| ENST00000 | — | — | — | — | — | Detected | — | — | — |
| 307428.7_1_ | | | | | | | | | |
| 4:75858563- | | | | | | | | | |
| 75937719:+ | | | | | | | | | |
| ENST00000 | — | — | — | — | — | Detected | — | — | — |
| 537473.2_1_ | | | | | | | | | |
| 12:5705810 | | | | | | | | | |
| 3- | | | | | | | | | |
| 57058217:- | | | | | | | | | |
| ENST00000 | — | — | — | — | — | Detected | — | — | — |
| 429060.1_1_ | | | | | | | | | |
| 6:8784460- | | | | | | | | | |
| 8785356:+ | | | | | | | | | |
| ENST00000 | — | — | — | — | — | Detected | — | — | — |
| 430069.5_1_ | | | | | | | | | |
| 3:17677167 | | | | | | | | | |
| 6- | | | | | | | | | |
| 176782752:- | | | | | | | | | |
| ENST00000 | — | — | — | — | — | Detected | — | — | — |
| 410062.8_1_ | | | | | | | | | |
| 2:18184675 | | | | | | | | | |
| 5- | | | | | | | | | |
| 181848786: | | | | | | | | | |
| + | | | | | | | | | |
| ENST00000 | — | — | — | — | — | Detected | — | — | — |
| 344823.9_1_ | | | | | | | | | |
| 16:3451289- | | | | | | | | | |
| 3451709:+ | | | | | | | | | |
| ENST00000 | — | — | — | — | — | Detected | — | — | — |
| 466942.2_1_ | | | | | | | | | |
| 10:7716730 | | | | | | | | | |
| 5- | | | | | | | | | |
| 77167401:+ | | | | | | | | | |
| ENST00000 | — | — | — | — | — | Detected | — | — | — |
| 507606.2_1_ | | | | | | | | | |
| 7:13534728 | | | | | | | | | |
| 3- | | | | | | | | | |
| 135358947: | | | | | | | | | |
| + | | | | | | | | | |
| ENST00000 | — | — | — | — | — | Detected | — | — | — |
| 409011.5_2_ | | | | | | | | | |
| 2:39005336- | | | | | | | | | |
| 39005417:+ | | | | | | | | | |
| ENST00000 | — | — | — | — | — | Detected | — | — | — |
| 299505.6_1_ | | | | | | | | | |
| 19:3479111 | | | | | | | | | |
| 8- | | | | | | | | | |
| 34791184:+ | | | | | | | | | |
| ENST00000 | — | — | — | — | — | Detected | — | — | — |
| 446618.1_1_ | | | | | | | | | |
| 22:1924587 | | | | | | | | | |
| 9- | | | | | | | | | |
| 19246326:+ | | | | | | | | | |
| ENST00000 | — | — | — | — | — | Detected | — | — | — |
| 380338.8_1_ | | | | | | | | | |
| 9:20622330- | | | | | | | | | |
| 20622507:- | | | | | | | | | |
| ENST00000 | — | — | — | — | — | Detected | — | — | — |
| 253247.8_1_ | | | | | | | | | |
| 17:6573210 | | | | | | | | | |
| 3- | | | | | | | | | |
| 65732808:+ | | | | | | | | | |
| ENST00000 | — | — | — | — | Detected | — | — | — | |
| 514616.5_1_ | | | | | | | | | |
| 5:13722385 | | | | | | | | | |
| 5- | | | | | | | | | |
| 137224842:- | | | | | | | | | |
| ENST00000 | — | — | — | — | — | Detected | — | — | — |
| 480739.2_1_ | | | | | | | | | |
| 12:5806887 | | | | | | | | | |
| 7- | | | | | | | | | |
| 58069030:+ | | | | | | | | | |
| ENST00000 | — | — | — | — | — | Detected | — | — | — |
| 510737.1_1_ | | | | | | | | | |
| 4:12016039 | | | | | | | | | |
| 5- | | | | | | | | | |
| 120160458: | | | | | | | | | |
| + | | | | | | | | | |
| ENST00000 | — | — | — | — | — | Detected | — | — | — |
| 613266.4_1_ | | | | | | | | | |
| 15:8043021 | | | | | | | | | |
| 2- | | | | | | | | | |
| 80430356:+ | | | | | | | | | |
| ENST00000 | — | — | — | — | — | Detected | — | — | — |
| 398571.6_2_ | | | | | | | | | |
| 2:61633207- | | | | | | | | | |
| 61647956:- | | | | | | | | | |
| ENST00000 | — | — | — | — | Detected | Detected | — | — | — |
| 372894.7_1_ | | | | | | | | | |
| 20:4316052 | | | | | | | | | |
| 1- | | | | | | | | | |
| 43218474:+ | | | | | | | | | |
| ENST00000 | — | — | — | — | Detected | Detected | — | — | — |
| 429705.6_1_ | | | | | | | | | |
| 3:43328077- | | | | | | | | | |
| 43344643:+ | | | | | | | | | |
| ENST00000 | — | — | — | — | Detected | Detected | — | — | — |
| 347770.8_1_ | | | | | | | | | |
| 8:10385200 | | | | | | | | | |
| 5- | | | | | | | | | |
| 103852041:- | | | | | | | | | |
| ENST00000 | — | — | — | — | Detected | Detected | — | — | — |
| 298767.9_1_ | | | | | | | | | |
| 10:8827755 | | | | | | | | | |
| 8- | | | | | | | | | |
| 88277621:- | | | | | | | | | |
| ENST00000 | — | — | — | — | Detected | Detected | — | — | — |
| 546387.1_1_ | | | | | | | | | |
| 12:4423858 | | | | | | | | | |
| 1- | | | | | | | | | |
| 44238653+ | | | | | | | | | |
| ENST00000 | — | Detected | — | — | Detected | Detected | — | — | — |
| 457540.1_1_ | | | | | | | | | |
| 1:565028- | | | | | | | | | |
| 565094:+ | | | | | | | | | |
| ENST00000 | — | — | Detected | — | Detected | Detected | — | — | — |
| 229390.7_1_ | | | | | | | | | |
| 12:1209018 | | | | | | | | | |
| 92- | | | | | | | | | |
| 120907330:- | | | | | | | | | |
| ENST00000 | — | Detected | Detected | — | Detected | Detected | — | — | — |
| 559345.5_1_ | | | | | | | | | |
| 15:7916554 | | | | | | | | | |
| 8- | | | | | | | | | |
| 79172860:+ | | | | | | | | | |
| ENST00000 | — | — | — | — | — | — | Detected | — | — |
| 362000.9_1_ | | | | | | | | | |
| 7:65540928- | | | | | | | | | |
| 65547388:+ | | | | | | | | | |
| ENST00000 | — | — | — | — | — | — | Detected | — | — |
| 615047.4_1_ | | | | | | | | | |
| 19:4427842 | | | | | | | | | |
| 8- | | | | | | | | | |
| 44285151:- | | | | | | | | | |
| ENST00000 | — | — | — | — | — | — | Detected | — | — |
| 259873.4_1_ | | | | | | | | | |
| 6:30587521- | | | | | | | | | |
| 30590615:+ | | | | | | | | | |
| ENST00000 | — | — | — | — | — | — | Detected | — | — |
| 493719.5_1_ | | | | | | | | | |
| 13:2801456 | | | | | | | | | |
| 9- | | | | | | | | | |
| 28024213:- | | | | | | | | | |
| ENST00000 | — | — | — | — | — | — | Detected | — | — |
| 394464.6_1_ | | | | | | | | | |
| 5:14278041 | | | | | | | | | |
| 6- | | | | | | | | | |
| 142782924:- | | | | | | | | | |
| ENST00000 | — | — | — | — | — | — | Detected | — | — |
| 438124.2_1_ | | | | | | | | | |
| 11:6850517 | | | | | | | | | |
| 8- | | | | | | | | | |
| 68506175:- | | | | | | | | | |
| ENST00000 | — | — | — | — | — | — | Detected | — | — |
| 256015.4_1_ | | | | | | | | | |
| 12:9253953 | | | | | | | | | |
| 5- | | | | | | | | | |
| 92539610:- | | | | | | | | | |
| ENST00000 | — | — | — | — | — | — | Detected | — | — |
| 440177.6_1_ | | | | | | | | | |
| 19:4275345 | | | | | | | | | |
| 2- | | | | | | | | | |
| 42753692:- | | | | | | | | | |
| ENST00000 | — | — | — | — | — | — | Detected | — | — |
| 267068.5_2_ | | | | | | | | | |
| 13:3311079 | | | | | | | | | |
| 1- | | | | | | | | | |
| 33110824:- | | | | | | | | | |
| ENST00000 | — | — | — | — | — | — | Detected | — | — |
| 368875.6_2_ | | | | | | | | | |
| 1:15129865 | | | | | | | | | |
| 4- | | | | | | | | | |
| 151299818:- | | | | | | | | | |
| ENST00000 | Detected | — | — | — | — | — | Detected | — | — |
| 327247.9_1_ | | | | | | | | | |
| 1:15521049 | | | | | | | | | |
| 2- | | | | | | | | | |
| 155214452:- | | | | | | | | | |
| T026338_1: | — | — | — | — | — | Detected | Detected | — | — |
| 183227158- | | | | | | | | | |
| 183228074: | | | | | | | | | |
| + | | | | | | | | | |
| ENST00000 | — | — | — | — | — | Detected | Detected | — | — |
| 559725.5_1_ | | | | | | | | | |
| 15:6065669 | | | | | | | | | |
| 2- | | | | | | | | | |
| 60682421:- | | | | | | | | | |
| ENST00000 | — | — | — | — | — | Detected | Detected | — | — |
| 343327.6_1_ | | | | | | | | | |
| 9:11503045 | | | | | | | | | |
| 2- | | | | | | | | | |
| 115095785:- | | | | | | | | | |
| ENST00000 | — | — | — | — | — | Detected | Detected | — | — |
| 283632.4_1_ | | | | | | | | | |
| 2:86968058- | | | | | | | | | |
| 86968091:+ | | | | | | | | | |
| ENST00000 | — | — | — | — | — | — | — | Detected | — |
| 478253.5_1_ | | | | | | | | | |
| 3:58223274- | | | | | | | | | |
| 58223340:+ | | | | | | | | | |
| ENST00000 | — | — | — | — | — | — | — | Detected | — |
| 571543.1_1_ | | | | | | | | | |
| 17:1131443- | | | | | | | | | |
| 1132291:- | | | | | | | | | |
| ENST00000 | — | — | — | — | — | — | — | Detected | — |
| 440938.1_1_ | | | | | | | | | |
| 2:17435059 | | | | | | | | | |
| 5- | | | | | | | | | |
| 174351021:- | | | | | | | | | |
| ENST00000 | — | — | — | — | — | — | — | Detected | — |
| 418351.1_1_ | | | | | | | | | |
| 15:4428126 | | | | | | | | | |
| 1- | | | | | | | | | |
| 44281585:- | | | | | | | | | |
| ENST00000 | — | — | — | — | — | — | — | Detected | — |
| 553814.5_1_ | | | | | | | | | |
| 14:7453824 | | | | | | | | | |
| 9- | | | | | | | | | |
| 74551097:- | | | | | | | | | |
| ENST00000 | — | — | — | — | — | — | — | Detected | — |
| 495856.1_1_ | | | | | | | | | |
| 1:95507120- | | | | | | | | | |
| 95538397:- | | | | | | | | | |
| ENST00000 | — | — | — | — | — | — | — | Detected | — |
| 447713.1_3_ | | | | | | | | | |
| 2:19800137 | | | | | | | | | |
| 4- | | | | | | | | | |
| 198175430:- | | | | | | | | | |
| ENST00000 | — | — | — | — | — | — | — | Detected | — |
| 463678.5_1_ | | | | | | | | | |
| 1:11002868 | | | | | | | | | |
| 6- | | | | | | | | | |
| 110035219: | | | | | | | | | |
| + | | | | | | | | | |
| ENST00000 | — | — | — | — | — | — | — | Detected | — |
| 294244.8_1_ | | | | | | | | | |
| 11:6359448 | | | | | | | | | |
| 7- | | | | | | | | | |
| 63594532:+ | | | | | | | | | |
| ENST00000 | — | — | — | — | — | — | — | Detected | — |
| 272139.4_1_ | | | | | | | | | |
| 1:22828917 | | | | | | | | | |
| 1- | | | | | | | | | |
| 228290722:- | | | | | | | | | |
| ENST00000 | — | — | — | — | — | — | — | Detected | — |
| 511208.2_1_ | | | | | | | | | |
| 5:31532460- | | | | | | | | | |
| 3 1535865:+ | | | | | | | | | |
| ENST00000 | — | — | — | — | — | — | — | Detected | — |
| 429344.6_1_ | | | | | | | | | |
| 19:3623927- | | | | | | | | | |
| 3626666:- | | | | | | | | | |
| ENST00000 | — | — | — | — | — | — | — | Detected | — |
| 528088.1_1_ | | | | | | | | | |
| 11:1188812 | | | | | | | | | |
| 37- | | | | | | | | | |
| 118882689: | | | | | | | | | |
| + | | | | | | | | | |
| ENST00000 | — | — | — | — | — | — | — | Detected | — |
| 579344.1_1_ | | | | | | | | | |
| 17:3871078 | | | | | | | | | |
| 7- | | | | | | | | | |
| 38710997:- | | | | | | | | | |
| ENST00000 | — | — | — | — | — | — | — | Detected | — |
| 430443.1_1_ | | | | | | | | | |
| 3:19043291 | | | | | | | | | |
| 6- | | | | | | | | | |
| 190432991: | | | | | | | | | |
| + | | | | | | | | | |
| ENST00000 | — | — | — | — | — | — | — | Detected | — |
| 518797.5_2_ | | | | | | | | | |
| 5:14978149 | | | | | | | | | |
| 3- | | | | | | | | | |
| 149781793:- | | | | | | | | | |
| ENST00000 | — | — | — | — | — | — | — | Detected | — |
| 588498.1_1_ | | | | | | | | | |
| 19:1050600 | | | | | | | | | |
| 0- | | | | | | | | | |
| 10506648:- | | | | | | | | | |
| ENST00000 | — | — | — | — | — | — | — | Detected | — |
| 508756.2_1_ | | | | | | | | | |
| 5:98204705- | | | | | | | | | |
| 98207835:- | | | | | | | | | |
| ENST00000 | — | — | — | — | — | — | — | Detected | — |
| 574446.1_1_ | | | | | | | | | |
| 4:10379022 | | | | | | | | | |
| 6- | | | | | | | | | |
| 103803968: | | | | | | | | | |
| + | | | | | | | | | |
| ENST00000 | — | — | — | — | — | — | — | Detected | — |
| 572125.1_1_ | | | | | | | | | |
| 16:4408902- | | | | | | | | | |
| 4409034:- | | | | | | | | | |
| ENST00000 | — | — | — | — | — | — | — | Detected | — |
| 333750.9_1_ | | | | | | | | | |
| 12:3724520- | | | | | | | | | |
| 3753802:- | | | | | | | | | |
| ENST00000 | — | — | — | — | — | — | — | Detected | — |
| 217131.5_1_ | | | | | | | | | |
| 20:5757268 | | | | | | | | | |
| 8- | | | | | | | | | |
| 57576600:- | | | | | | | | | |
| ENST00000 | — | — | — | — | — | — | — | Detected | — |
| 591095.1_1_ | | | | | | | | | |
| 17:7670567 | | | | | | | | | |
| 1- | | | | | | | | | |
| 76732801:- | | | | | | | | | |
| ENST00000 | — | — | — | — | — | — | — | Detected | — |
| 452733.6_1_ | | | | | | | | | |
| 19:4897248 | | | | | | | | | |
| 9- | | | | | | | | | |
| 48972630:+ | | | | | | | | | |
| ENST00000 | — | — | — | — | — | — | — | Detected | — |
| 450119.1_1_ | | | | | | | | | |
| 10:9281337 | | | | | | | | | |
| 7- | | | | | | | | | |
| 92813905:+ | | | | | | | | | |
| ENST00000 | — | — | — | — | — | — | — | Detected | — |
| 381341.6_1_ | | | | | | | | | |
| 2:15753361- | | | | | | | | | |
| 15757371:+ | | | | | | | | | |
| ENST00000 | — | — | — | — | — | — | — | Detected | — |
| 587730.5_1_ | | | | | | | | | |
| 19:1452081 | | | | | | | | | |
| 1- | | | | | | | | | |
| 14521537:- | | | | | | | | | |
| ENST00000 | — | — | — | — | — | — | — | Detected | — |
| 534802.1_1_ | | | | | | | | | |
| 11:4640023 | | | | | | | | | |
| 4- | | | | | | | | | |
| 46401494:+ | | | | | | | | | |
| ENST00000 | — | — | — | — | — | — | — | Detected | — |
| 483757.5_1_ | | | | | | | | | |
| 9:273070- | | | | | | | | | |
| 365624:+ | | | | | | | | | |
| ENST00000 | — | — | — | — | — | — | — | Detected | — |
| 312515.6_1_ | | | | | | | | | |
| 11:6568684 | | | | | | | | | |
| 5- | | | | | | | | | |
| 65687846:+ | | | | | | | | | |
| ENST00000 | — | — | — | — | — | — | — | Detected | — |
| 567658.1_1_ | | | | | | | | | |
| 16:2714480 | | | | | | | | | |
| 6- | | | | | | | | | |
| 27145163:- | | | | | | | | | |
| ENST00000 | — | — | — | — | — | — | — | Detected | — |
| 428356.1_1_ | | | | | | | | | |
| 10:9442853 | | | | | | | | | |
| 1- | | | | | | | | | |
| 94429500:- | | | | | | | | | |
| ENST00000 | — | — | — | — | — | — | — | Detected | — |
| 460378.1_1_ | | | | | | | | | |
| 1:29422780- | | | | | | | | | |
| 29423143:+ | | | | | | | | | |
| ENST00000 | — | — | — | — | — | — | — | Detected | — |
| 474216.5_1_ | | | | | | | | | |
| 1:11126777- | | | | | | | | | |
| 11137033:- | | | | | | | | | |
| ENST00000 | — | — | — | — | — | — | — | Detected | — |
| 549321.1_1_ | | | | | | | | | |
| 12:1118011 | | | | | | | | | |
| 90- | | | | | | | | | |
| 111803987:- | | | | | | | | | |
| ENST00000 | — | — | — | — | — | — | — | Detected | — |
| 632314.l_l_ | | | | | | | | | |
| 10:1372682 | | | | | | | | | |
| 9- | | | | | | | | | |
| 13749113:- | | | | | | | | | |
| ENST00000 | — | — | — | — | — | — | — | Detected | — |
| 490202.5_1_ | | | | | | | | | |
| X:48339880- | | | | | | | | | |
| 48341236:+ | | | | | | | | | |
| ENST00000 | — | — | — | — | — | — | — | Detected | — |
| 638093.1_1_ | | | | | | | | | |
| 6:16647806 | | | | | | | | | |
| 2- | | | | | | | | | |
| 166478587: | | | | | | | | | |
| + | | | | | | | | | |
| ENST00000 | — | — | — | — | — | — | — | Detected | — |
| 510396.1_1_ | | | | | | | | | |
| 4:15259380 | | | | | | | | | |
| 8- | | | | | | | | | |
| 152594048:- | | | | | | | | | |
| ENST00000 | — | — | — | — | — | — | — | Detected | — |
| 343632.8_1_ | | | | | | | | | |
| 22:3802893 | | | | | | | | | |
| 5- | | | | | | | | | |
| 38029157:+ | | | | | | | | | |
| ENST00000 | — | — | — | — | — | — | — | Detected | — |
| 492349.5_1_ | | | | | | | | | |
| 3:52720087- | | | | | | | | | |
| 52722217:+ | | | | | | | | | |
| ENST00000 | — | — | — | — | — | — | — | Detected | — |
| 368232.8_1_ | | | | | | | | | |
| 1:15656477 | | | | | | | | | |
| 8- | | | | | | | | | |
| 156565066:- | | | | | | | | | |
| ENST00000 | — | — | — | — | — | — | — | Detected | — |
| 376861.5_1_ | | | | | | | | | |
| 6:29693814- | | | | | | | | | |
| 29694269:+ | | | | | | | | | |
| ENST00000 | — | — | — | — | — | — | — | Detected | — |
| 358344.7_1_ | | | | | | | | | |
| 5:17763759 | | | | | | | | | |
| 8- | | | | | | | | | |
| 177637649: | | | | | | | | | |
| + | | | | | | | | | |
| ENST00000 | — | — | — | — | — | — | — | Detected | — |
| 399515.2_1_ | | | | | | | | | |
| 2:19078806 | | | | | | | | | |
| 1- | | | | | | | | | |
| 190788865: | | | | | | | | | |
| + | | | | | | | | | |
| ENST00000 | — | — | — | — | — | — | — | Detected | — |
| 528764.1_1_ | | | | | | | | | |
| 11:8673566 | | | | | | | | | |
| 4- | | | | | | | | | |
| 86735814:+ | | | | | | | | | |
| ENST00000 | — | — | — | — | — | — | — | Detected | — |
| 490442.5_1_ | | | | | | | | | |
| 10:7009823 | | | | | | | | | |
| 3- | | | | | | | | | |
| 70101119:+ | | | | | | | | | |
| ENST00000 | — | — | — | — | — | — | — | Detected | — |
| 530115.1_1_ | | | | | | | | | |
| 11:2791155 | | | | | | | | | |
| 1- | | | | | | | | | |
| 27912580:- | | | | | | | | | |
| ENST00000 | — | — | — | — | — | — | — | Detected | — |
| 522493.5_1_ | | | | | | | | | |
| 8:25297162- | | | | | | | | | |
| 25298149:- | | | | | | | | | |
| ENST00000 | — | — | — | — | — | — | — | Detected | — |
| 374403.3_1_ | | | | | | | | | |
| X:69595194- | | | | | | | | | |
| 69596002:+ | | | | | | | | | |
| ENST00000 | — | — | — | — | — | — | — | Detected | — |
| 488600.1_1_ | | | | | | | | | |
| 1:21200264 | | | | | | | | | |
| 0- | | | | | | | | | |
| 212003596:- | | | | | | | | | |
| ENST00000 | — | — | — | — | — | — | — | Detected | — |
| 556423.1_1_ | | | | | | | | | |
| 14:7106331 | | | | | | | | | |
| 0- | | | | | | | | | |
| 71067354:- | | | | | | | | | |
| ENST00000 | — | — | — | — | — | — | — | Detected | — |
| 361899.2_M | | | | | | | | | |
| T:8530- | | | | | | | | | |
| 8572:+ | | | | | | | | | |
| ENST00000 | — | — | — | — | — | — | — | Detected | — |
| 361453.3_M | | | | | | | | | |
| T:5228- | | | | | | | | | |
| 5411:+ | | | | | | | | | |
| ENST00000 | — | — | — | — | — | — | — | Detected | — |
| 259523.10_1_ | | | | | | | | | |
| 8:1287477 | | | | | | | | | |
| 12- | | | | | | | | | |
| 128748060: | | | | | | | | | |
| + | | | | | | | | | |
| ENST00000 | — | — | — | — | — | — | — | Detected | — |
| 488254.6_1_ | | | | | | | | | |
| 10:1039220 | | | | | | | | | |
| 35- | | | | | | | | | |
| 103922146: | | | | | | | | | |
| + | | | | | | | | | |
| ENST00000 | — | — | — | — | — | — | — | Detected | — |
| 473525.1_1_ | | | | | | | | | |
| X:70516758- | | | | | | | | | |
| 70517827:+ | | | | | | | | | |
| ENST00000 | — | — | — | — | — | — | — | Detected | — |
| 558123.5_1_ | | | | | | | | | |
| 15:4164832 | | | | | | | | | |
| 0- | | | | | | | | | |
| 41650442:+ | | | | | | | | | |
| ENST00000 | — | — | — | — | — | — | — | Detected | — |
| 336066.7_1_ | | | | | | | | | |
| 20:3870065- | | | | | | | | | |
| 3891231:+ | | | | | | | | | |
| ENST00000 | — | — | — | — | — | — | — | Detected | — |
| 570059.1_1_ | | | | | | | | | |
| 15:5561143 | | | | | | | | | |
| 3- | | | | | | | | | |
| 55619742:+ | | | | | | | | | |
| ENST00000 | — | — | — | — | — | — | — | Detected | — |
| 612501.1_1_ | | | | | | | | | |
| 17:4822736 | | | | | | | | | |
| 7- | | | | | | | | | |
| 48227940:- | | | | | | | | | |
| ENST00000 | — | — | — | — | — | — | — | Detected | — |
| 533331.5_1_ | | | | | | | | | |
| 11:1453511 | | | | | | | | | |
| 0- | | | | | | | | | |
| 14539190:- | | | | | | | | | |
| ENST00000 | — | — | — | — | — | — | — | Detected | — |
| 339488.8_2_ | | | | | | | | | |
| 6:48036008- | | | | | | | | | |
| 48036398:- | | | | | | | | | |
| ENST00000 | — | — | — | — | — | — | — | Detected | — |
| 403542.6_1_ | | | | | | | | | |
| 20:3432844 | | | | | | | | | |
| 7- | | | | | | | | | |
| 34329909:- | | | | | | | | | |
| ENST00000 | — | — | — | — | — | — | — | Detected | — |
| 433031.1_1_ | | | | | | | | | |
| 7:57216803- | | | | | | | | | |
| 57217046:- | | | | | | | | | |
| ENST00000 | — | — | — | — | — | — | — | Detected | — |
| 399720.4_1_ | | | | | | | | | |
| 4:49490601- | | | | | | | | | |
| 49491475:- | | | | | | | | | |
| ENST00000 | — | — | — | — | — | — | — | Detected | — |
| 567565.3_1_ | | | | | | | | | |
| 15:7603928 | | | | | | | | | |
| 7- | | | | | | | | | |
| 76039500:+ | | | | | | | | | |
| ENST00000 | — | — | — | — | — | — | — | Detected | — |
| 435137.1_1_ | | | | | | | | | |
| 9:14204246- | | | | | | | | | |
| 14204684:+ | | | | | | | | | |
| ENST00000 | — | — | — | — | — | — | — | Detected | — |
| 435269.1_1_ | | | | | | | | | |
| 3:17409552 | | | | | | | | | |
| 5- | | | | | | | | | |
| 174095792: | | | | | | | | | |
| + | | | | | | | | | |
| ENST00000 | — | — | — | — | — | — | — | Detected | — |
| 609975.1_1_ | | | | | | | | | |
| 2:96191991- | | | | | | | | | |
| 96192402:- | | | | | | | | | |
| ENST00000 | — | — | — | — | — | — | — | Detected | — |
| 411837.1_1_ | | | | | | | | | |
| 1:45453908- | | | | | | | | | |
| 45454397:- | | | | | | | | | |
| ENST00000 | — | — | — | — | — | — | — | Detected | — |
| 431607.1_1_ | | | | | | | | | |
| 9:11994298 | | | | | | | | | |
| 3- | | | | | | | | | |
| 119943337:- | | | | | | | | | |
| ENST00000 | — | — | — | — | — | — | — | Detected | — |
| 509785.5_1_ | | | | | | | | | |
| 5:16901541 | | | | | | | | | |
| 4- | | | | | | | | | |
| 169017783: | | | | | | | | | |
| + | | | | | | | | | |
| ENST00000 | — | — | — | — | — | — | — | Detected | — |
| 466342.1_1_ | | | | | | | | | |
| 5:17925091 | | | | | | | | | |
| 6- | | | | | | | | | |
| 179260288: | | | | | | | | | |
| + | | | | | | | | | |
| ENST00000 | — | — | — | — | — | — | — | Detected | — |
| 541731.1_1_ | | | | | | | | | |
| 12:1603550 | | | | | | | | | |
| 0- | | | | | | | | | |
| 16042896:+ | | | | | | | | | |
| ENST00000 | — | — | — | — | — | — | — | Detected | — |
| 473429.5_1_ | | | | | | | | | |
| 1:11058436 | | | | | | | | | |
| 3- | | | | | | | | | |
| 110592127: | | | | | | | | | |
| + | | | | | | | | | |
| ENST00000 | — | — | — | — | — | — | — | Detected | — |
| 371142.8_1_ | | | | | | | | | |
| 10:9833657 | | | | | | | | | |
| 8- | | | | | | | | | |
| 98346758:- | | | | | | | | | |
| ENST00000 | — | — | — | — | — | — | — | Detected | — |
| 587504.5_1_ | | | | | | | | | |
| 17:7383599 | | | | | | | | | |
| 5- | | | | | | | | | |
| 73839803:- | | | | | | | | | |
| ENST00000 | — | — | — | — | — | — | — | Detected | — |
| 557884.5_1_ | | | | | | | | | |
| 15:6379681 | | | | | | | | | |
| 5- | | | | | | | | | |
| 63821292:+ | | | | | | | | | |
| ENST00000 | — | — | — | — | — | — | — | Detected | — |
| 418510.1_1_ | | | | | | | | | |
| 22:2604461 | | | | | | | | | |
| 7- | | | | | | | | | |
| 26045175:+ | | | | | | | | | |
| ENST00000 | — | — | — | — | — | — | — | Detected | — |
| 594019.5_1_ | | | | | | | | | |
| 19:4758866 | | | | | | | | | |
| 0- | | | | | | | | | |
| 47615436:- | | | | | | | | | |
| ENST00000 | — | — | — | — | — | — | — | Detected | — |
| 266557.3_1_ | | | | | | | | | |
| 12:6554080- | | | | | | | | | |
| 6554389:+ | | | | | | | | | |
| ENST00000 | — | — | — | — | — | — | — | Detected | — |
| 456708.5_1_ | | | | | | | | | |
| 2:22009021 | | | | | | | | | |
| 1- | | | | | | | | | |
| 220094280:- | | | | | | | | | |
| ENST00000 | — | — | — | — | Detected | — | — | Detected | — |
| 421406.1_1_ | | | | | | | | | |
| 1:24735328 | | | | | | | | | |
| 2- | | | | | | | | | |
| 247372753:- | | | | | | | | | |
| ENST00000 | — | — | — | — | — | — | — | — | Detected |
| 326685.11_1_ | | | | | | | | | |
| 5:1485211 | | | | | | | | | |
| 29- | | | | | | | | | |
| 148521582: | | | | | | | | | |
| + | | | | | | | | | |
| ENST00000 | — | — | — | — | — | — | — | — | Detected |
| 609979.1_1_ | | | | | | | | | |
| 7:10738380 | | | | | | | | | |
| 8- | | | | | | | | | |
| 107384430:- | | | | | | | | | |
| ENST00000 | — | — | — | — | — | — | — | — | Detected |
| 442739.1_1_ | | | | | | | | | |
| 22:2135724 | | | | | | | | | |
| 1- | | | | | | | | | |
| 21363382:- | | | | | | | | | |
| ENST00000 | — | — | — | — | — | — | — | — | Detected |
| 564693.1_1_ | | | | | | | | | |
| 15:3080937 | | | | | | | | | |
| 2- | | | | | | | | | |
| 30809693:+ | | | | | | | | | |
| ENST00000 | — | — | — | — | — | — | — | — | Detected |
| 554781.5_1_ | | | | | | | | | |
| 14:2480045 | | | | | | | | | |
| 8- | | | | | | | | | |
| 24803934:- | | | | | | | | | |
| ENST00000 | — | — | — | — | — | — | — | — | Detected |
| 453623.5_1_ | | | | | | | | | |
| 7:16684294- | | | | | | | | | |
| 16685213:- | | | | | | | | | |
| ENST00000 | — | — | — | — | — | — | — | — | Detected |
| 470032.1_1_ | | | | | | | | | |
| 5:14241676 | | | | | | | | | |
| 8- | | | | | | | | | |
| 142421157: | | | | | | | | | |
| + | | | | | | | | | |
| ENST00000 | — | — | — | — | — | — | — | — | Detected |
| 503013.2_1_ | | | | | | | | | |
| 4:11486743 | | | | | | | | | |
| 6- | | | | | | | | | |
| 114891654:- | | | | | | | | | |
| ENST00000 | — | — | — | — | — | — | — | — | Detected |
| 573167.2_1_ | | | | | | | | | |
| 17:7900704 | | | | | | | | | |
| 2- | | | | | | | | | |
| 79008646:- | | | | | | | | | |
| ENST00000 | — | — | — | — | — | — | — | — | Detected |
| 592895.5_1_ | | | | | | | | | |
| 19:1985822- | | | | | | | | | |
| 1986326:- | | | | | | | | | |
| ENST00000 | — | — | — | — | — | — | — | — | Detected |
| 298892.9_1_ | | | | | | | | | |
| 12:3090732 | | | | | | | | | |
| 0- | | | | | | | | | |
| 30907440:- | | | | | | | | | |
| ENST00000 | — | — | — | — | — | — | — | — | Detected |
| 635618.1_3_ | | | | | | | | | |
| 6:25610361- | | | | | | | | | |
| 25619808:+ | | | | | | | | | |
| ENST00000 | — | — | — | — | — | — | — | — | Detected |
| 590083.5_1_ | | | | | | | | | |
| 19:1228816- | | | | | | | | | |
| 1229167:- | | | | | | | | | |
| ENST00000 | — | — | — | — | — | — | — | — | Detected |
| 263398.10_1_ | | | | | | | | | |
| 11:352521 | | | | | | | | | |
| 96- | | | | | | | | | |
| 35252259:+ | | | | | | | | | |
| ENST00000 | — | — | — | — | — | — | — | — | Detected |
| 502249.6_1_ | | | | | | | | | |
| 4:11940152 | | | | | | | | | |
| 4- | | | | | | | | | |
| 119435218: | | | | | | | | | |
| + | | | | | | | | | |
| ENST00000 | — | — | — | — | — | — | — | — | Detected |
| 573745.1_1_ | | | | | | | | | |
| 17:7165530- | | | | | | | | | |
| 7166136:- | | | | | | | | | |
| ENST00000 | — | — | — | — | — | — | — | — | Detected |
| 620157.4_1_ | | | | | | | | | |
| 11:1186571 | | | | | | | | | |
| 61- | | | | | | | | | |
| 118661620:- | | | | | | | | | |
| ENST00000 | — | — | — | — | — | — | — | — | Detected |
| 463886.1_1_ | | | | | | | | | |
| 22:4199862 | | | | | | | | | |
| 8- | | | | | | | | | |
| 41998772:- | | | | | | | | | |
| ENST00000 | — | — | — | — | — | — | — | — | Detected |
| 254322.2_1_ | | | | | | | | | |
| 19:1462566 | | | | | | | | | |
| 3- | | | | | | | | | |
| 14625702:- | | | | | | | | | |
| ENST00000 | — | — | — | — | — | — | — | — | Detected |
| 497493.1_1_ | | | | | | | | | |
| 21:3859747 | | | | | | | | | |
| 4- | | | | | | | | | |
| 38597519:- | | | | | | | | | |
| ENST00000 | — | — | — | — | — | — | — | — | Detected |
| 636308.1_1_ | | | | | | | | | |
| 7:76607904- | | | | | | | | | |
| 76634926:+ | | | | | | | | | |
| ENST00000 | — | — | — | — | — | — | — | — | Detected |
| 325349.6_1_ | | | | | | | | | |
| 12:1714401 | | | | | | | | | |
| 7- | | | | | | | | | |
| 17144188:+ | | | | | | | | | |
| ENST00000 | — | — | — | — | — | — | — | — | Detected |
| 419025.1_1_ | | | | | | | | | |
| 3:18374501 | | | | | | | | | |
| 2- | | | | | | | | | |
| 183745345:- | | | | | | | | | |
| ENST00000 | — | — | — | — | — | — | — | — | Detected |
| 517956.5_1_ | | | | | | | | | |
| 8:12451464 | | | | | | | | | |
| 3- | | | | | | | | | |
| 124514769:- | | | | | | | | | |
| ENST00000 | — | — | — | — | — | — | — | — | Detected |
| 615526.1_1_ | | | | | | | | | |
| 20:6218526 | | | | | | | | | |
| 2- | | | | | | | | | |
| 62185625:+ | | | | | | | | | |
| ENST00000 | — | — | — | — | — | — | — | — | Detected |
| 162391.7_1_ | | | | | | | | | |
| 12:8185349- | | | | | | | | | |
| 8185487:+ | | | | | | | | | |
| ENST00000 | — | — | — | — | — | — | — | — | Detected |
| 395675.4_1_ | | | | | | | | | |
| 17:1857562 | | | | | | | | | |
| 4- | | | | | | | | | |
| 18576137:- | | | | | | | | | |
| ENST00000 | — | — | — | — | — | — | — | — | Detected |
| 489495.5_1_ | | | | | | | | | |
| 1:78411296- | | | | | | | | | |
| 78412276:- | | | | | | | | | |
| ENST00000 | — | — | — | — | — | — | — | — | Detected |
| 481383.1_1_ | | | | | | | | | |
| 3:18068809 | | | | | | | | | |
| 1- | | | | | | | | | |
| 180688984: | | | | | | | | | |
| + | | | | | | | | | |
| ENST00000 | — | — | — | — | — | — | — | — | Detected |
| 507324.1_1_ | | | | | | | | | |
| 15:6482135 | | | | | | | | | |
| 8- | | | | | | | | | |
| 64821781:- | | | | | | | | | |
| ENST00000 | — | — | — | — | — | — | — | — | Detected |
| 511530.1_1_ | | | | | | | | | |
| 5:17394061 | | | | | | | | | |
| 5- | | | | | | | | | |
| 173940888: | | | | | | | | | |
| + | | | | | | | | | |
| ENST00000 | — | — | — | — | — | — | — | — | Detected |
| 471443.1_1_ | | | | | | | | | |
| 2:19179630 | | | | | | | | | |
| 9- | | | | | | | | | |
| 191797409: | | | | | | | | | |
| + | | | | | | | | | |
| ENST00000 | — | — | — | — | — | — | — | — | Detected |
| 460402.5_1_ | | | | | | | | | |
| 22:1977623 | | | | | | | | | |
| 4- | | | | | | | | | |
| 19842274:- | | | | | | | | | |
| ENST00000 | — | — | — | — | — | — | — | — | Detected |
| 426882.5_1_ | | | | | | | | | |
| 6:30294481- | | | | | | | | | |
| 30294724:- | | | | | | | | | |
| ENST00000 | — | — | — | — | — | — | — | — | Detected |
| 414046.2_1_ | | | | | | | | | |
| 6:31431445- | | | | | | | | | |
| 31431841:+ | | | | | | | | | |
| ENST00000 | — | — | — | — | — | — | — | — | Detected |
| 299192.7_1_ | | | | | | | | | |
| 16:5010409 | | | | | | | | | |
| 2- | | | | | | | | | |
| 50104164:+ | | | | | | | | | |
| ENST00000 | — | — | — | — | — | — | — | — | Detected |
| 585077.1_1_ | | | | | | | | | |
| 17:8039790 | | | | | | | | | |
| 7- | | | | | | | | | |
| 80398237:+ | | | | | | | | | |
| ENST00000 | — | — | — | — | — | — | — | — | Detected |
| 452680.3_1_ | | | | | | | | | |
| 1:11639959 | | | | | | | | | |
| 3- | | | | | | | | | |
| 116399968:- | | | | | | | | | |
| ENST00000 | — | — | — | — | — | — | — | — | Detected |
| 544000.5_1_ | | | | | | | | | |
| 10:4389007 | | | | | | | | | |
| 8- | | | | | | | | | |
| 43892652:- | | | | | | | | | |
| ENST00000 | — | — | — | — | — | — | — | — | Detected |
| 304858.6_1_ | | | | | | | | | |
| 5:13244007 | | | | | | | | | |
| 8- | | | | | | | | | |
| 132440150: | | | | | | | | | |
| + | | | | | | | | | |
| ENST00000 | — | — | — | — | — | — | — | — | Detected |
| 337478.2_1_ | | | | | | | | | |
| 10:1060754 | | | | | | | | | |
| 71- | | | | | | | | | |
| 106098072:- | | | | | | | | | |
| ENST00000 | — | — | — | — | — | — | — | — | Detected |
| 375377.1_1_ | | | | | | | | | |
| 10:3033654 | | | | | | | | | |
| 2- | | | | | | | | | |
| 30336710:- | | | | | | | | | |
| ENST00000 | — | — | — | — | — | — | — | — | Detected |
| 486361.5_1_ | | | | | | | | | |
| 7:73508538- | | | | | | | | | |
| 73511422:+ | | | | | | | | | |
| ENST00000 | — | — | — | — | — | — | — | — | Detected |
| 467673.5_1_ | | | | | | | | | |
| 1:39571193- | | | | | | | | | |
| 39720158:+ | | | | | | | | | |
| ENST00000 | — | — | — | — | — | — | — | — | Detected |
| 594582.1_1_ | | | | | | | | | |
| 19:4921852 | | | | | | | | | |
| 1- | | | | | | | | | |
| 49220230:- | | | | | | | | | |
| ENST00000 | — | — | — | — | — | — | — | — | Detected |
| 482752.1_1_ | | | | | | | | | |
| 3:47918864- | | | | | | | | | |
| 47969739:- | | | | | | | | | |
| ENST00000 | — | — | — | — | — | — | — | — | Detected |
| 434837.7_1_ | | | | | | | | | |
| 6:37665242- | | | | | | | | | |
| 37665689:- | | | | | | | | | |
| ENST00000 | — | — | — | — | — | — | — | — | Detected |
| 223215.8_1_ | | | | | | | | | |
| 7:13013195 | | | | | | | | | |
| 8- | | | | | | | | | |
| 130132015: | | | | | | | | | |
| + | | | | | | | | | |
| ENST00000 | — | — | — | — | — | — | — | — | Detected |
| 301618.8_1_ | | | | | | | | | |
| 17:7486473 | | | | | | | | | |
| 8- | | | | | | | | | |
| 74864864:+ | | | | | | | | | |
| ENST00000 | — | — | — | — | — | — | — | — | Detected |
| 467199.5_1_ | | | | | | | | | |
| 7:2274759- | | | | | | | | | |
| 2281791:- | | | | | | | | | |
| ENST00000 | — | — | — | — | — | — | — | — | Detected |
| 570986.1_1_ | | | | | | | | | |
| 17:4457199- | | | | | | | | | |
| 4458649:- | | | | | | | | | |
| ENST00000 | — | — | — | — | — | — | — | — | Detected |
| 545054.6_1_ | | | | | | | | | |
| 14:2456358 | | | | | | | | | |
| 3- | | | | | | | | | |
| 24563937:+ | | | | | | | | | |
| ENST00000 | — | — | — | — | — | — | — | — | Detected |
| 314222.4_1_ | | | | | | | | | |
| 11:2949785- | | | | | | | | | |
| 2950526:- | | | | | | | | | |
| ENST00000 | — | — | — | — | — | — | — | — | Detected |
| 292140.9_1_ | | | | | | | | | |
| 19:4400812 | | | | | | | | | |
| 1- | | | | | | | | | |
| 44008847:- | | | | | | | | | |
| ENST00000 | — | — | — | — | — | — | — | — | Detected |
| 587482.1_1_ | | | | | | | | | |
| 19:3649915- | | | | | | | | | |
| 3700388:- | | | | | | | | | |
| ENST00000 | — | — | — | — | — | — | — | — | Detected |
| 552429.5_1_ | | | | | | | | | |
| 12:4272001 | | | | | | | | | |
| 8- | | | | | | | | | |
| 42768464:+ | | | | | | | | | |
| ENST00000 | — | — | — | — | — | — | — | — | Detected |
| 588556.1_1_ | | | | | | | | | |
| 16:4942976- | | | | | | | | | |
| 4943674:- | | | | | | | | | |
| ENST00000 | — | — | — | — | — | — | — | — | Detected |
| 612688.1_1_ | | | | | | | | | |
| 2:55842578- | | | | | | | | | |
| 55844368:- | | | | | | | | | |
| ENST00000 | — | — | — | — | — | — | — | — | Detected |
| 308677.8_1_ | | | | | | | | | |
| 19:1422847 | | | | | | | | | |
| 4- | | | | | | | | | |
| 14228519:- | | | | | | | | | |
| ENST00000 | — | — | — | — | — | — | — | — | Detected |
| 397497.8_2_ | | | | | | | | | |
| 8:27288464- | | | | | | | | | |
| 27288860:+ | | | | | | | | | |
| ENST00000 | — | — | — | — | — | — | — | — | Detected |
| 361758.8_1_ | | | | | | | | | |
| 6:16383620 | | | | | | | | | |
| 9- | | | | | | | | | |
| 163876314: | | | | | | | | | |
| + | | | | | | | | | |
| ENST00000 | — | — | — | — | — | — | — | — | Detected |
| 418705.2_1_ | | | | | | | | | |
| 22:2010573 | | | | | | | | | |
| 6- | | | | | | | | | |
| 20106546:+ | | | | | | | | | |
| ENST00000 | — | — | — | — | — | — | — | — | Detected |
| 552916.5_1_ | | | | | | | | | |
| 12:5691579 | | | | | | | | | |
| 5- | | | | | | | | | |
| 56963704:+ | | | | | | | | | |
| ENST00000 | — | — | — | — | — | — | — | — | Detected |
| 423293.1_1_ | | | | | | | | | |
| 22:4147093 | | | | | | | | | |
| 1- | | | | | | | | | |
| 41471243:- | | | | | | | | | |
| ENST00000 | — | — | — | — | — | — | — | — | Detected |
| 403475.2_1_ | | | | | | | | | |
| 6:84102419- | | | | | | | | | |
| 84102602:+ | | | | | | | | | |
| ENST00000 | — | — | — | — | — | — | — | — | Detected |
| 321285.4_1_ | | | | | | | | | |
| 4:11348614 | | | | | | | | | |
| 3- | | | | | | | | | |
| 113486335: | | | | | | | | | |
| + | | | | | | | | | |
| ENST00000 | — | — | — | — | — | — | — | — | Detected |
| 608279.1_1_ | | | | | | | | | |
| 2:13310438 | | | | | | | | | |
| 0- | | | | | | | | | |
| 133104455:- | | | | | | | | | |
| ENST00000 | — | — | — | — | — | — | — | — | Detected |
| 550612.1_1_ | | | | | | | | | |
| 12:3101492 | | | | | | | | | |
| 3- | | | | | | | | | |
| 31015109:+ | | | | | | | | | |
| ENST00000 | — | — | — | — | — | — | — | — | Detected |
| 546846.1_1_ | | | | | | | | | |
| 12:4798701 | | | | | | | | | |
| 1- | | | | | | | | | |
| 47987203:+ | | | | | | | | | |
| ENST00000 | — | — | — | — | — | — | — | — | Detected |
| 369367.7_2_ | | | | | | | | | |
| 12:4635794 | | | | | | | | | |
| 6- | | | | | | | | | |
| 46384197:- | | | | | | | | | |
| ENST00000 | — | — | — | — | — | — | — | — | Detected |
| 356994.6_1_ | | | | | | | | | |
| 8:14489605 | | | | | | | | | |
| 6- | | | | | | | | | |
| 144897480:- | | | | | | | | | |
| ENST00000 | — | — | — | — | — | — | — | — | Detected |
| 600523.5_1_ | | | | | | | | | |
| 1:24969796- | | | | | | | | | |
| 24973575:+ | | | | | | | | | |
| ENST00000 | — | — | — | — | — | — | — | — | Detected |
| 630499.2_1_ | | | | | | | | | |
| 16:2802712- | | | | | | | | | |
| 2807817:+ | | | | | | | | | |
| ENST00000 | — | — | — | — | — | — | — | — | Detected |
| 176763.9_1_ | | | | | | | | | |
| 5:17147127 | | | | | | | | | |
| 4- | | | | | | | | | |
| 171471448:- | | | | | | | | | |
| ENST00000 | — | — | — | — | — | — | — | — | Detected |
| 372774.7_2_ | | | | | | | | | |
| X:10139570 | | | | | | | | | |
| 4- | | | | | | | | | |
| 101395815:- | | | | | | | | | |
| ENST00000 | — | — | — | — | — | — | — | — | Detected |
| 444939.1_1_ | | | | | | | | | |
| 3:19605068 | | | | | | | | | |
| 4- | | | | | | | | | |
| 196051038: | | | | | | | | | |
| + | | | | | | | | | |
| ENST00000 | — | — | — | — | — | — | — | — | Detected |
| 233143.5_1_ | | | | | | | | | |
| 2:85133515- | | | | | | | | | |
| 85133596:+ | | | | | | | | | |
| ENST00000 | — | — | — | — | — | — | — | — | Detected |
| 458118.5_1_ | | | | | | | | | |
| 13:4142888 | | | | | | | | | |
| 5- | | | | | | | | | |
| 41438465:- | | | | | | | | | |
| ENST00000 | — | — | — | — | — | — | — | — | Detected |
| 518096.1_1_ | | | | | | | | | |
| 8:30210219- | | | | | | | | | |
| 30210855:+ | | | | | | | | | |
| ENST00000 | — | — | — | — | — | — | — | — | Detected |
| 399822.2_1_ | | | | | | | | | |
| 9:22012279- | | | | | | | | | |
| 22012411:+ | | | | | | | | | |
| ENST00000 | — | — | — | — | — | — | — | — | Detected |
| 440317.1_1_ | | | | | | | | | |
| 2:12731520 | | | | | | | | | |
| 8- | | | | | | | | | |
| 127315727:- | | | | | | | | | |
| ENST00000 | — | — | — | — | — | — | — | — | Detected |
| 596172.1_1_ | | | | | | | | | |
| 19:8935711- | | | | | | | | | |
| 8942974:- | | | | | | | | | |
| ENST00000 | — | — | — | — | — | — | — | — | Detected |
| 595784.1_1_ | | | | | | | | | |
| 19:5859827 | | | | | | | | | |
| 1- | | | | | | | | | |
| 58598791:- | | | | | | | | | |
| TCONS_00 | — | — | — | — | — | — | — | — | Detected |
| 001132_1:1 | | | | | | | | | |
| 21138933- | | | | | | | | | |
| 121169919: | | | | | | | | | |
| + | | | | | | | | | |
| T000395_1: | — | — | — | — | — | — | — | — | Detected |
| 1002569- | | | | | | | | | |
| 1003328:- | | | | | | | | | |
| T093792_13: | — | — | — | — | — | — | — | — | Detected |
| 44881128- | | | | | | | | | |
| 44881524:+ | | | | | | | | | |
| T234938_22: | — | — | — | — | — | — | — | — | Detected |
| 44419815- | | | | | | | | | |
| 44420103:- | | | | | | | | | |
| T236099_22: | — | — | — | — | — | — | — | — | Detected |
| 50328122- | | | | | | | | | |
| 50328950:- | | | | | | | | | |
| T236101_22: | — | — | — | — | — | — | — | — | Detected |
| 50328365- | | | | | | | | | |
| 50329190:- | | | | | | | | | |
| T325348_7: | — | — | — | — | — | — | — | — | Detected |
| 64769928- | | | | | | | | | |
| 64770336:+ | | | | | | | | | |
| T339715_8: | — | — | — | — | — | — | — | — | Detected |
| 17701253- | | | | | | | | | |
| 17705101:- | | | | | | | | | |
| T348749_8: | — | — | — | — | — | — | — | — | Detected |
| 103151442- | | | | | | | | | |
| 103152410:- | | | | | | | | | |
| ENST00000 | — | — | — | — | — | — | — | — | Detected |
| 367996.5_1_ | | | | | | | | | |
| 1:16116074 | | | | | | | | | |
| 2- | | | | | | | | | |
| 161160913:- | | | | | | | | | |
| ENST00000 | — | — | — | — | — | — | — | — | Detected |
| 479540.5_1_ | | | | | | | | | |
| 20:6220345 | | | | | | | | | |
| 6- | | | | | | | | | |
| 62204512:- | | | | | | | | | |
| ENST00000 | — | — | — | — | — | — | — | Detected | Detected |
| 5672991_1_ | | | | | | | | | |
| 19:9628825- | | | | | | | | | |
| 9629221:+ | | | | | | | | | |
| ENST00000 | — | — | — | — | — | — | — | Detected | Detected |
| 571636.1_1_ | | | | | | | | | |
| 16:1133655 | | | | | | | | | |
| 3- | | | | | | | | | |
| 11336613:+ | | | | | | | | | |
| ENST00000 | — | — | — | — | — | — | — | Detected | Detected |
| 597731.1_1_ | | | | | | | | | |
| 19:3932736 | | | | | | | | | |
| 4- | | | | | | | | | |
| 39331218:- | | | | | | | | | |
| ENST00000 | — | — | — | — | — | — | — | Detected | Detected |
| 429127.2_1_ | | | | | | | | | |
| 12:8167969- | | | | | | | | | |
| 8168583:+ | | | | | | | | | |
| ENST00000 | — | — | — | — | — | — | — | Detected | Detected |
| 342160.7_2_ | | | | | | | | | |
| X:53630437- | | | | | | | | | |
| 53631569:- | | | | | | | | | |
| ENST00000 | — | — | — | — | — | — | — | Detected | Detected |
| 579309.1_1_ | | | | | | | | | |
| 17:2578381 | | | | | | | | | |
| 0- | | | | | | | | | |
| 25810713:+ | | | | | | | | | |
| ENST00000 | — | — | — | — | — | — | — | Detected | Detected |
| 536528.5_1_ | | | | | | | | | |
| 11:1841615 | | | | | | | | | |
| 9- | | | | | | | | | |
| 18424398:+ | | | | | | | | | |
| ENST00000 | — | — | — | — | — | — | — | Detected | Detected |
| 464120.1_1_ | | | | | | | | | |
| 22:3807167 | | | | | | | | | |
| 0- | | | | | | | | | |
| 38073073:+ | | | | | | | | | |
| ENST00000 | — | — | — | — | — | — | — | Detected | Detected |
| 397487.3_1_ | | | | | | | | | |
| 2:13238357 | | | | | | | | | |
| 9- | | | | | | | | | |
| 132384836: | | | | | | | | | |
| + | | | | | | | | | |
| ENST00000 | — | — | — | — | — | — | — | Detected | Detected |
| 487452.1_1_ | | | | | | | | | |
| 7:10003355 | | | | | | | | | |
| 0- | | | | | | | | | |
| 100034039:- | | | | | | | | | |
| ENST00000 | — | — | — | — | — | — | — | Detected | Detected |
| 635248.1_1_ | | | | | | | | | |
| 1:17412872 | | | | | | | | | |
| 6- | | | | | | | | | |
| 174212140: | | | | | | | | | |
| + | | | | | | | | | |
| ENST00000 | — | — | — | — | — | — | — | Detected | Detected |
| 465850.1_1_ | | | | | | | | | |
| 3:11382209 | | | | | | | | | |
| 8- | | | | | | | | | |
| 113822344:- | | | | | | | | | |
| ENST00000 | — | — | — | — | — | — | — | Detected | Detected |
| 498682.2_3_ | | | | | | | | | |
| 7:15010468 | | | | | | | | | |
| 1- | | | | | | | | | |
| 150105308: | | | | | | | | | |
| + | | | | | | | | | |
| ENST00000 | — | — | — | — | — | — | — | Detected | Detected |
| 600588.1_1_ | | | | | | | | | |
| 19:6593418- | | | | | | | | | |
| 6593715:+ | | | | | | | | | |
| ENST00000 | — | — | — | — | — | — | — | Detected | Detected |
| 401730.5_1_ | | | | | | | | | |
| 19:4958877 | | | | | | | | | |
| 4- | | | | | | | | | |
| 49605396:+ | | | | | | | | | |
| ENST00000 | — | — | — | — | — | — | — | Detected | Detected |
| 483815.5_1_ | | | | | | | | | |
| 20:3523435 | | | | | | | | | |
| 1- | | | | | | | | | |
| 35236338:+ | | | | | | | | | |
| ENST00000 | — | — | — | Detected | — | — | — | Detected | Detected |
| 403734.2_1_ | | | | | | | | | |
| 11:6249435 | | | | | | | | | |
| 9- | | | | | | | | | |
| 62494803:- | | | | | | | | | |
| ENST00000 | — | — | — | Detected | — | — | — | Detected | Detected |
| 624069.3_1_ | | | | | | | | | |
| 19:4999087 | | | | | | | | | |
| 7- | | | | | | | | | |
| 49993734:+ | | | | | | | | | |

There was a strong agreement between the peptides presented in the cancer cells and in the HLA-matched B721.221 models (FIG. 34E, 34F) for both annotated ORFs and nuORFs. The extent of overlap increased with the increase in the number of HLA alleles matching between B721.221 and the cancer cells (FIG. 34G), for both annotated peptides (Pearson r²=0.87, p=10⁻³⁰) and nuORFs (r²=0.70, p=10⁻¹⁷). Those ORFs that were detected in cancer cells but not in B721.221 cells had a lower level of translation in B721.221 cells, for both annotated ORFs (p=10⁻¹⁶², t-test) and nuORFs (p<10′, t-test) (FIG. 34H).

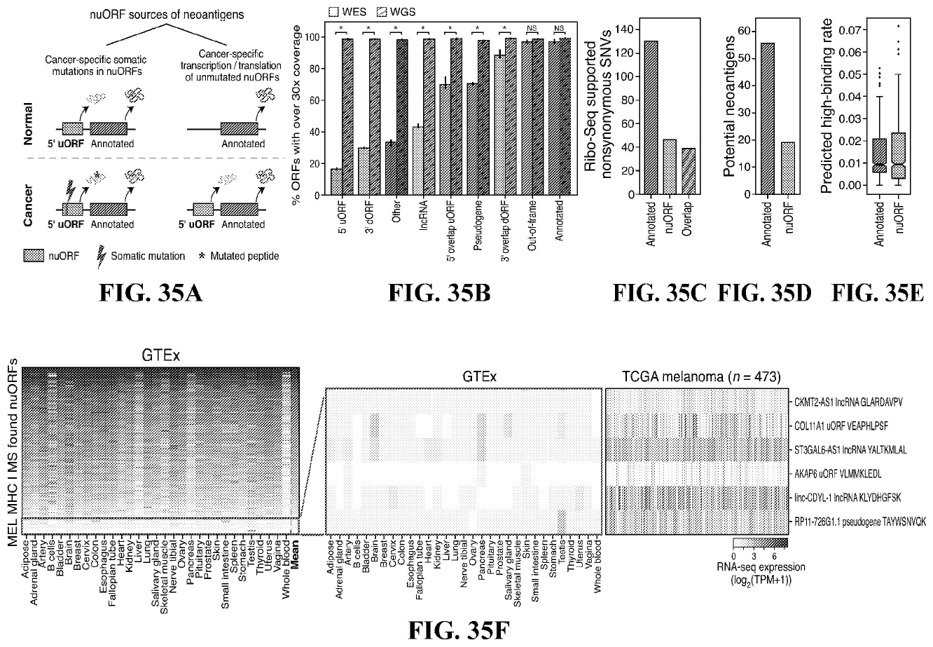

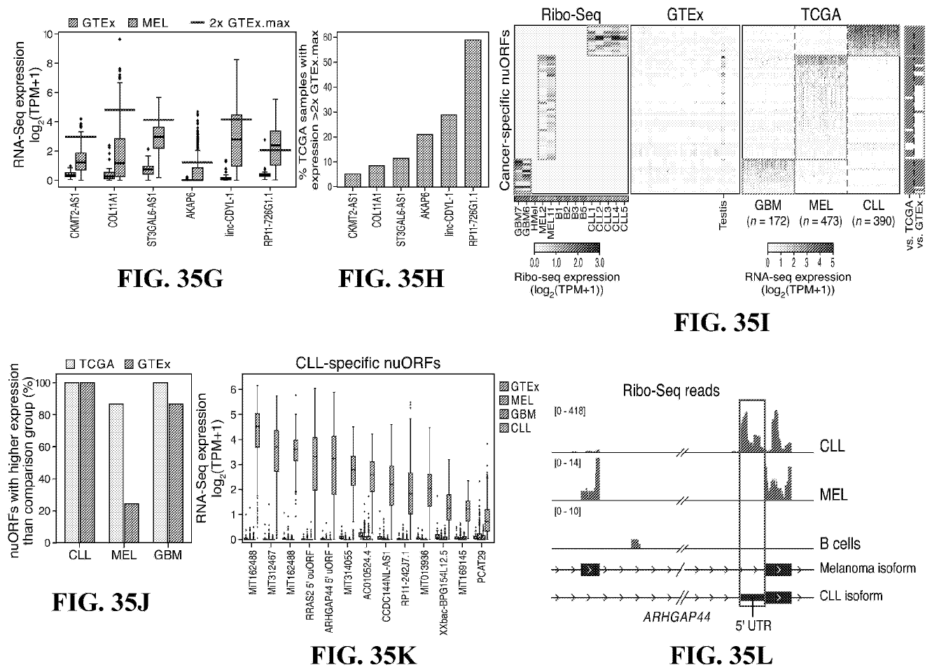

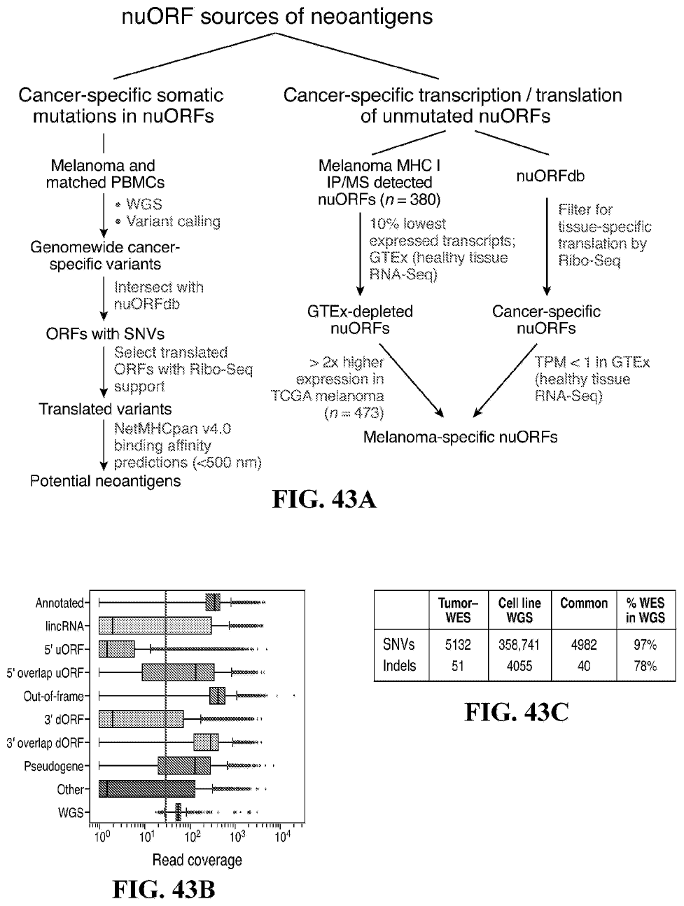

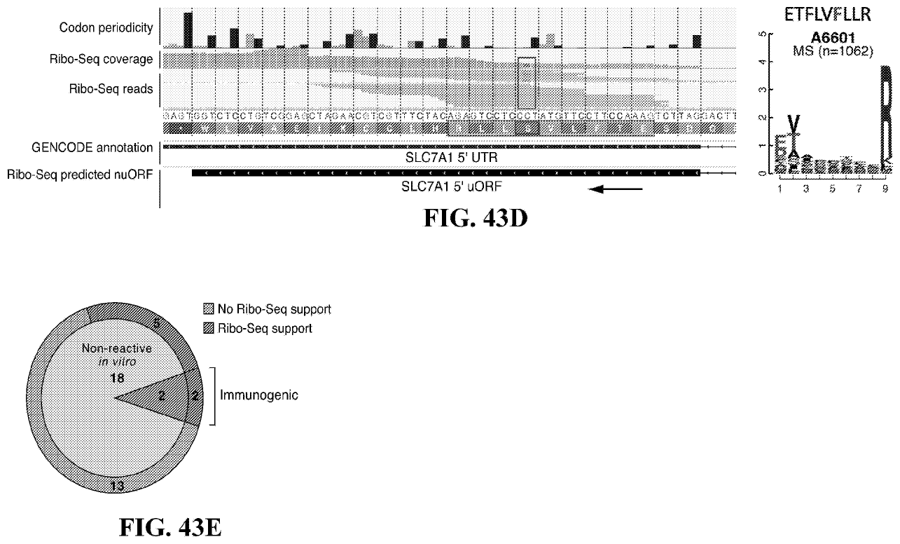

Next, Applicants estimated the extent to which nuORFs can serve as cancer neoantigens through either (1) cancer-specific somatic mutations in nuORFs; or (2) translation in a cancer-specific manner (FIG. 35A, FIG. 43A).

For the first category, Applicants considered that WES is currently the standard approach to identify cancer-specific somatic mutations in annotated ORFs, yet it does not provide sufficient coverage to capture somatic mutations across various categories of nuORFs. While >99% of canonical ORFs had over the recommended 30× median coverage in WES, the coverage across nuORF types varied, and only 19.5% of 5′uORFs and 43% of nuORF-bearing lncRNAs had similar coverage in WES (FIG. 35B, FIG. 43B, SOM). Applicants therefore performed WGS, which provided at least 30× median coverage of over 98% of both annotated ORFs and nuORFs, across all types (FIG. 35B, FIG. 43B).

To estimate the potential contribution of nuORFs with somatic mutations to the neoantigen repertoire in the systems. Applicants focused the WGS analysis on a primary melanoma cell line (and matched PBMCs) previously characterized by WES, obtained from a patient who had received a personal neoantigen-targeting cancer vaccine (4); these cells were further profiled by Ribo-seq. The somatic mutation analysis by WGS closely recapitulated the original patient tumor genetics profiled by WES (FIG. 43C). Applicants developed a computational pipeline to obtain the Ribo-seq translation support for the mutant and wild-type alleles containing single nucleotide variants (SNVs) (FIG. 43D, SOM). Applicants then selected the most likely neoantigens that could be derived from somatic variants in nuORFs and annotated ORFs, by prioritizing those that are: (1) mutated (by somatic mutation calling), (2) translated (by Ribo-seq coverage, in the mutant allele), and (3) MHC I presented (<500 nM predicted MHC I binding affinity by netMHCpan 4.0 (28)). When Applicants re-analyzed the variants previously selected for the neoantigen vaccine in this patient (4), Applicants found that 2 out of 2 neoantigens that elicited an immune response in the patient had Ribo-seq support, but only 5 of 18 (28%) of those that were not immunogenic had Ribo-seq support, highlighting the potential utility of Ribo-seq in prioritizing translated variants (FIG. 43E).

Overall, for this patient sample, 19 of 75 candidate neoantigens (25%), identified by WGS analysis and MHC class I binding predictions based on the patient's HLA alleles, were derived from translated nuORFs. Specifically, Ribo-seq supported the translation of 217 SNVs, 22% of them exclusively in nuORFs (FIG. 35C). These results suggest that nuORFs are a viable source of additional potential neoantigens in cancer (FIG. 35C, 35D). Given the diversity of HLA alleles across the human population, Applicants estimate that the rate at which mutations can generate antigens with high predicted MHC I binding affinity across alleles is 1.4% and 1.6% for annotated ORFs and nuORFs, respectively (FIG. 35E, CI 95%: 0.1-0.3%).

Applicants also assessed the potential for neoantigen generation by cancer specific translation. To identify nuORFs translated in a melanoma-specific manner, Applicants first analyzed the 381 nuORFs detected in the MHC I immunopeptidomes from 6 melanoma samples (FIG. 35F, Table 1) and identified 6 high confidence melanoma-specific nuORF candidates (Table 2A-2B). These Applicants defined as those transcripts among the 381 nuORFs that were both lowly expressed across all healthy tissues (except the testis) in The Genotype-Tissue Expression (GTEx) collection of healthy tissues (29) (FIG. 35F (red box)), and expressed at least 2-fold higher than GTEx in at least 5% of 473 melanoma samples in The Cancer Genome Atlas (TCGA) (30, 31) (FIG. 35A, 35F, SOM). Two of these six nuORFs, found in the RP11-726G1.1 pseudogene and the Zinc-CDYL-1 lncRNA, were highly overexpressed in 28% and 59% of TCGA melanoma samples respectively, suggesting a potential utility for many melanoma patients (FIG. 35G, 35H).

TABLE 2A
MS-based MEL

| ORF_ID | geneName | plotType | mergeType |
|---|---|---|---|
| ENST00000488132.5_1_3:98434244- | ST3GAL6- | lncRNA | Antisense |
| 98434552: - | AS1 | | |
| ENST00000512287.1_1_5:80531836- | CKMT2- | lncRNA | Antisense |
| 80531875:- | AS1 | | |
| TCONS_00011153_6:4611322- | linc- | lncRNA | lincRNA |
| 4611508:+ | CDYL-1 | | |
| ENST00000370096.7_1_1:103574000- | COL11A1 | 5′ uORF | 5′ uORF |
| 103574030:- | | | |
| ENST00000554410.5_1_14:32963479- | AKAP6 | 5′ uORF | 5′ uORF |
| 32963518:+ | | | |
| ENST00000640129.1_1_12:9723336- | RP11- | Out-of- | Out-of- |
| 9723429:+ | 726G1.1 | Frame | Frame |

TABLE 2B

| Tumor | | Gene | Plot | Merge | Versus | Versus |
|---|---|---|---|---|---|---|
| Type | ORF_ID | Name | Type | Type | TCGA | GTEx |
| Riboseq- | ENST00000583608.1_1_17:12859099- | ARHGAP44 | 5′ uORF | 5′ uORF | PASS | PASS |
| based | 12859180:+ | | | | | |
| CLL | ENST00000423473.6_1_17:20899806- | CCDC144NL- | lncRNA | Antisense | PASS | PASS |
| | 20901188:+ | AS1 | | | | |
| | ENST00000611838.1_1_6:32223085- | XXbac- | lncRNA | Antisense | PASS | PASS |
| | 32317317:+ | BPG154L12.5 | | | | |
| | T013936_1:83945334-83945391:- | mitrans | lncRNA | lincRNA | PASS | PASS |
| | T162485_18:52673962-52762449:- | mitrans | lncRNA | lincRNA | PASS | PASS |
| | T162488_18:52746328-52762365:- | mitrans | lncRNA | lincRNA | PASS | PASS |
| | T314055_6:156615822-156615870:- | mitrans | lncRNA | lincRNA | PASS | PASS |
| | ENST00000559212.1_1_15:69973631- | PCAT29 | lncRNA | lincRNA | PASS | PASS |
| | 69988065:+ | | | | | |
| | T169145_19:13215208-13215725:+ | mitrans | Other | TUCP | PASS | PASS |
| | ENST00000531807.5_1_11:14363366- | RRAS2 | 5′ Overlap | 5′ Overlap | PASS | PASS |
| | 14363504:- | | uORF | uORF | | |
| | ENST00000522554.1_1_4:185546006- | RP11-242J7.1 | lncRNA | lincRNA | PASS | PASS |
| | 185546117:- | | | | | |
| | T312467_6:139750544-139750634:+ | mitrans | lncRNA | lincRNA | PASS | PASS |
| | ENST00000596488.1_1_19:49859474- | AC010524.4 | lncRNA | Antisense | PASS | PASS |
| | 49860832:+ | | | | | |
| Riboseq- | ENST00000407071.6_2_1:245318369- | KIF26B | 5′ uORF | 5′ uORF | PASS | PASS |
| based | 245318450:+ | | | | | |
| MEL | ENST00000550711.1_1_12:104849191- | CHST11 | lncRNA | ncRNA | — | — |
| | 104849392:+ | | | Processed | | |
| | | | | Transcript | | |
| | ENST00000295226.1_1_2:223169047- | CCDC140 | Canonical | Truncated | PASS | PASS |
| | 223169110:+ | | | | | |
| | TCONS_00029529_22:23851980- | linc-RGL4-6 | lncRNA | lincRNA | PASS | — |
| | 23852055:+ | | | | | |
| | ENST00000462675.1_1_3:75540221- | ENPP7P2 | Pseudogene | Pseudogene | — | — |
| | 75540299:- | | | | | |
| | ENST00000508853.1_1_5:38148581- | LINC02119 | lncRNA | lincRNA | PASS | PASS |
| | 38148647:+ | | | | | |
| | ENST00000380970.2_1_7:53833907- | LINC01446 | lncRNA | lincRNA | PASS | — |
| | 53879616:- | | | | | |
| | TCONS_00017243_X:124453968- | linc-CXorf64- | Canonical | Canonical | PASS | — |
| | 124456947:+ | 2 | | | | |
| | T035001_1:248469443-248469632:+ | mitrans | lncRNA | lincRNA | PASS | PASS |
| | T035748_10:3373607-3373718:+ | mitrans | lncRNA | lincRNA | PASS | — |
| | T095146_13:55613962-55637045+ | mitrans | lncRNA | lincRNA | PASS | — |
| | T095151_13:55628578-55637045:+ | mitrans | lncRNA | lincRNA | PASS | — |
| | T192655_2:81709114-81775762:+ | mitrans | lncRNA | lincRNA | PASS | PASS |
| | T206559_2:208924727-208937046:+ | mitrans | lncRNA | lincRNA | PASS | — |
| | T206563_2:208929469-208937046:+ | mitrans | lncRNA | lincRNA | PASS | — |
| | T220065_20:50956914-50957025:+ | mitrans | lncRNA | lincRNA | PASS | — |
| | T257106_3:191430385-191497326:+ | mitrans | lncRNA | lincRNA | PASS | — |
| | T257107_3:191430385-191501910:+ | mitrans | lncRNA | lincRNA | PASS | — |
| | T269973_4:115068324-115068836:+ | mitrans | lncRNA | lincRNA | PASS | — |
| | T280212_5:28078042-28084058:+ | mitrans | lncRNA | lincRNA | PASS | — |
| | T312199_6:138014181-138019867:+ | mitrans | lncRNA | lincRNA | PASS | PASS |
| | T333716_7:134396289-134453187:+ | mitrans | lncRNA | lincRNA | PASS | — |
| | T349874_8:116594833-116594884:+ | mitrans | lncRNA | lincRNA | — | — |
| | ENST00000616578.1_1_13:55637129- | RP11- | lncRNA | lincRNA | PASS | — |
| | 55680923:+ | 78L16.1 | | | | |
| | T035001_1:248469615-248469690:+ | mitrans | lncRNA | lincRNA | PASS | PASS |
| | T333717_7:134398878-134453113:+ | mitrans | lncRNA | lincRNA | PASS | — |
| | T333717_7:134396259-134398879:+ | mitrans | lncRNA | lincRNA | PASS | — |
| | T257122_3:191430341-191477334:+ | mitrans | lncRNA | lincRNA | PASS | — |
| | T257106_3:191430341-191495054:+ | mitrans | lncRNA | lincRNA | PASS | — |
| | T192659_2:81709163-81709211:+ | mitrans | lncRNA | lincRNA | PASS | PASS |
| | T257107_3:191377069-191430242:+ | mitrans | lncRNA | lincRNA | — | — |
| | T314020_6:155827068-155827119:+ | mitrans | lncRNA | lincRNA | PASS | PASS |
| | ENST00000557481.6_1_15:94442527- | LINC01579 | lncRNA | lincRNA | PASS | — |
| | 94443685:- | | | | | |
| | ENST00000557715.1_1_15:94442527- | LINC01579 | lncRNA | lincRNA | PASS | — |
| | 94443301:- | | | | | |
| | ENST00000247452.3_1_X:141290961- | MAGEC2 | Out-of- | Out-of- | PASS | PASS |
| | 141291006:- | | Frame | Frame | | |
| | ENST00000593011.5_1_1:152140699- | FLG-AS1 | lncRNA | Antisense | — | — |
| | 152140738:+ | | | | | |
| | ENST00000437267.2_1:143186450- | RP11- | lncRNA | lincRNA | PASS | — |
| | 143186519:- | 782C8.2 | | | | |
| | ENST00000233638.7_1_2:74741778- | TLX2 | 5′ uORF | 5′ uORF | — | — |
| | 74741835:+ | | | | | |
| | ENST00000409910.1_1_2:238337821- | AC112721.2 | lncRNA | lincRNA | PASS | — |
| | 238343336:- | | | | | |
| | T257119_3:191430341-191444036:+ | mitrans | lncRNA | lincRNA | PASS | — |
| | ENST00000450877.5_1_7:111031942- | IMMP2L | Out-of- | Out-of- | PASS | PASS |
| | 111031978:- | | Frame | Frame | | |
| | ENST00000632314.1_1_10:13726607- | FRMD4A | lncRNA | ncRNA | PASS | — |
| | 13726712:- | | | Processed | | |
| | | | | Transcript | | |
| | ENST00000632314.1_1_10:13726312- | FRMD4A | lncRNA | ncRNA | PASS | — |
| | 13726411:- | | | Processed | | |
| | | | | Transcript | | |
| | ENST00000632314.1_1_10:13726326- | FRMD4A | lncRNA | ncRNA | PASS | — |
| | 13726350:- | | | Processed | | |
| | | | | Transcript | | |
| | ENST00000567333.1_1_16:34489847- | RARRES2P6 | Pseudogene | Pseudogene | PASS | — |
| | 34489919:- | | | | | |
| Riboseq- | ENST00000552667.5_1_12:113229403- | RPH3A | 5′ uORF | 5′ uORF | PASS | — |
| based | 113229965:+ | | | | | |
| GBM | ENST00000554105.5_1_15:93856435- | RP11- | lncRNA | lincRNA | PASS | PASS |
| | 93856513:+ | 266O8.1 | | | | |
| | ENST00000554105.5_1_15:93856528- | RP11- | lncRNA | lincRNA | PASS | PASS |
| | 93856573:+ | 266O8.1 | | | | |
| | T102657_14:33403596-33404077:- | mitrans | Other | TUCP | PASS | — |
| | T346776_8:82283541-82311962:+ | mitrans | lncRNA | lincRNA | PASS | PASS |
| | ENST00000373034.8_1_X:99664827- | PCDH19 | 5′ uORF | 5′ uORF | PASS | PASS |
| | 99665028:- | | | | | |
| | ENST00000544869.5_1_2:104669961- | LINC01965 | lncRNA | lincRNA | PASS | PASS |
| | 104694108:+ | | | | | |
| | ENST00000597347.5_1_3:180892826- | SOX2-OT | Other | Sense | PASS | PASS |
| | 180892943:+ | | | Overlapping | | |
| | ENST00000597347.5_1_3:180892882- | SOX2-OT | Other | Sense | PASS | PASS |
| | 180892957:+ | | | Overlapping | | |
| | ENST00000635290.1_1_1:61333589- | RP11- | lncRNA | lincRNA | PASS | PASS |
| | 61333625:- | 436K8.1 | | | | |
| | ENST00000404795.3_1_1:50888941- | DMRTA2 | 5′ uORF | 5′ uORF | PASS | PASS |
| | 50888974:- | | | | | |
| | ENST00000493116.5_1_3:180892960- | SOX2-OT | Other | Sense | PASS | PASS |
| | 180893017:+ | | | Overlapping | | |
| | ENST00000523244.5_1_8:26370361- | PNMA2 | lncRNA | ncRNA | PASS | PASS |
| | 26370445:- | | | Processed | | |
| | | | | Transcript | | |
| | ENST00000446923.6_1_10:31610338- | ZEB1 | 5′ uORF | 5′ uORF | PASS | PASS |
| | 31610398:+ | | | | | |
| | ENST00000551194.1_1_12:49687281- | PRPH | lncRNA | ncRNA | PASS | PASS |
| | 49687517:+ | | | Processed | | |
| | | | | Transcript | | |

TABLE 3A
MS-based Melanoma

| ORF_ID | geneName | plotType | mergeType | Peptide | sequence |
|---|---|---|---|---|---|
| ENST00000488132.5_1_3: | ST3GAL6- | lncRNA | Antisense | YALTK | LRMKPASEEDRQKGS |
| 98434244-98434552:− | AS1 | | | MLAL | GYIMSLKSILHTQLA |
| | | | | | AQNPDKPLIRNQPQT |
| | | | | | LYALTKMLALRTNYE |
| ENST00000512287.1_1_5: | CKMT2- | lncRNA | Antisense | GLARD | MCLPGLARDAVPV |
| 80531836-80531875:− | AS1 | | | AVPV | |
| TCONS_00011153_6: | line- | lncRNA | lincRNA | KLYDH | MRLRCFSTLHPLQHC |
| 4611322-4611508:+ | CDYL-1 | | | GFSK | LESQAWSQSYGLTLK |
| | | | | | RPLFIRREKGGRDEK |
| | | | | | GRQRKKLYDHGFSKI |
| | | | | | HG |
| ENST00000370096.7_1_1: | COL11A1 | 5′ | 5′ | VEAPH | VEAPHLPSF |
| 103574000-103574030:− | | uORF | uORF | LPSF | |
| ENST00000554410.5_1_14: | AKAP6 | 5′ | 5′ | VLMMK | MSRVLMMKLEDL |
| 32963479-32963518:+ | | uORF | uORF | LEDL | |
| ENST00000640129.1_1_12: | RP11- | Out-of- | Out-of- | TAYWS | IKTLSPLAVKDPVRF |
| 9723336-9723429:+ | 726G1.1 | Frame | Frame | NVQK | SRLTVITAYWSNVQK |

TABLE 3B
Riboseq-based Melanoma

| ORF_ID | geneName | plotType | mergeType | sequence |
|---|---|---|---|---|
| ENST00000407071.6_2_1: | KIF26B | 5′ | 5′ | LIVSLAFLVGEHGLTWLKKMPVLWMWP |
| 245318369-245318450:+ | | uORF | uORF | |
| ENST00000550711.1_1_12: | CHST11 | lncRNA | ncRNA | MRDLYSRQRLCANAHLSHALCKESPFL |
| 104849191-104849392:+ | | | Processed | VMMVGRWGGCSATPIPDLLSPEQKNHW |
| | | | Transcript | RTTLSEACSAWLP |
| ENST00000295226.1_1_2: | CCDC140 | Canonical | Truncated | MKKKKERKRRKERKKERNFKK |
| 223169047-223169110:+ | | | | |
| TCONS_00029529_22: | linc- | lncRNA | lincRNA | MHHLGCNYHSCLLRGHGGLEAAADS |
| 23851980-23852055:+ | RGL4-6 | | | |
| ENST00000462675.1_1_3: | ENPP7P2 | Pseudogene | Pseudogene | MWTPPNWTVRPGRASRPSTSCRPLSR |
| 75540221-75540299:− | | | | |
| ENST00000508853.1_1_5: | LINC02119 | lncRNA | lincRNA | VMRAFSESLVFCSVPSAALSHA |
| 38148581-38148647:+ | | | | |
| ENST00000380970.2_1_7: | LINC01446 | lncRNA | lincRNA | MADEQRAGFSPCGSLSLTVLGGCFLSC |
| 53833907-53879616:− | | | | CPWESDSRFFSLWPLGLSGLGGLTRST |
| | | | | RWASLVWGPGTWRSPAASVRPVLGVAL |
| | | | | KRQPVPQVSSLHTRLIACWFRPSAGFS |
| | | | | LPLSNSHTAERAYGRDEAVHSKNILIL |
| | | | | RITSPPGITGVLKKCCDTSEHREIHGH |
| | | | | FQIMEDSRRNFN |
| TCONS_00017243_X: | linc- | Canonical | Canonical | MAMNFGDHASGFRHDDVIRFINNEVLR |
| 124453968-124456947:+ | CXorf64-2 | | | NGGSPAFYTAFRSRPWNEVEDRLRAIV |
| | | | | ADPRVPRAIKRACTWSALALSVQVAAR |
| | | | | QQEELLYQVWWLQGHVEECQATSWALT |
| | | | | SQLQQLRLEHEEVATQLHLTQAALQQV |
| | | | | LNERDGLCGRLLEVERSMQVYPMPQDF |
| | | | | VPGPEAGQYGPVAGTLNAEQSEAVATE |
| | | | | AQGMPHSEAQVAAPTAVYYMPEPQSGR |
| | | | | VQGMQPLLLMQAPHPVPFHMPSPMGLP |
| | | | | YSTPLPPPVVMESAAAIAPQMPPAGIY |
| | | | | PPGLWATVGSQEETAPPWDQKCHGQDG |
| | | | | YPENFQGVYHPGDNRSCNQKEGSECPQ |
| | | | | GMTSQGDSSSHSLKKDPVMQEGTAPPE |
| | | | | FSRSHSLEKKPVMPKEMVPLGDSNSHS |
| | | | | LKKDPVVPKEIVPIGDSNSHSLTKNPV |
| | | | | VHKEMVSLGDSNSHSMKKDPVMPQKMV |
| | | | | PLGDSNSHSLKKDPMMCQEMVPLGDSN |
| | | | | SHSLKKDPVVAQGTAPLMYSRRHSQKK |
| | | | | VPMMPKEMVPLGESHSHSLKKDLVVPK |
| | | | | ELVPLGDSKSHRMKKDPVMPQKMVPLG |
| | | | | DSRSHSLKKDPVMPQNMIPLEDSNSHS |
| | | | | LKKDPVMPQNMIPLEDSNSHSLKKDPM |
| | | | | MHQEMVPLGDSNSHSLKKDPVVPQDTA |
| | | | | PLMFSRRHSLKKVPVMPKEMVPLGDSH |
| | | | | SLKKDPVMPQNMVPLEDSNSHSLKKDP |
| | | | | VVPQGTAPLMFSRRHSLKKVPVMPKEM |
| | | | | VPLGDSNSHSLKKDPVVPQGTAPLMFS |
| | | | | RRHSLKKVPVMPKEMVPLGDSHSLKKD |
| | | | | PVMPQNMVPLEDSNSHSLKKDPVVPQG |
| | | | | TAPLTFSRRHSLKKVPVVPQGTASLGF |
| | | | | SRIHSLKKELVMPEEMVPLGDSNSHSM |
| | | | | KKDLVMPKEMVPLGDSNSHSLKKDPVV |
| | | | | HQEVVSLGDSNSHSLKKHPVIPQGTAS |
| | | | | LRFSKSHSQKEDQERPQVTPLEDSKSH |
| | | | | GVKNSPWKHQPQGQKVKEQKRKKASES |
| | | | | QQQKPASCSSPVNWACPWCNAMNFPRN |
| | | | | KVCSKCKRVRMPVENGSVDPA |
| T035001_1: | mitrans | lncRNA | lincRNA | LHRICSVVSLEMSLDGDSSIPSYFCGA |
| 248469443-248469632:+ | | | | GGAEMGAKAESRKSCASGVQMLPFQRV |
| | | | | SLRHGAVLG |
| T035748_10: | mitrans | lncRNA | lincRNA | METAIPPVWMELNDTYNCRARLALPGL |
| 3373607-3373718:+ | | | | GNGRRGWRGH |
| T095146_13: | mitrans | lncRNA | lincRNA | VPSTMIMRSLTVLQIPDLK |
| 55613962-55637045:+ | | | | |
| T095151_13: | mitrans | lncRNA | lincRNA | VTSKAMRSLTVLQIPDLK |
| 55628578-55637045:+ | | | | |
| T192655_2: | mitrans | lncRNA | lincRNA | LTSCVTSRRKGLHRQLEGEQSGEVLSR |
| 81709114-81775762:+ | | | | EETGVSCPYPQVPTVIHQ |
| T206559_2: | mitrans | lncRNA | lincRNA | MVEGEVGAGTSHGKSKSKRERVKLLIS |
| 208924727-208937046:+ | | | | MEFLTSNISMKIITIYFLKFWEGMETS |
| | | | | HCSRCWGCSNEQDRPDPYSQINTMIQV |
| | | | | SRHLVGICIKLLGNGRQQH |
| T206563_2: | mitrans | lncRNA | lincRNA | LEAYRERPASWDTEQLLISMEFLTSNI |
| 208929469-208937046:+ | | | | SMKIITIYFLKFWEGMETSHCSRCWGC |
| | | | | SNEQDRPDPYSQINTMIQVSRHLVGIC |
| | | | | IKLLGNGRQQH |
| T220065_20: | mitrans | lncRNA | lincRNA | MARMWQPSGYNSVTNPSLRLPCNPSSA |
| 50956914-50957025:+ | | | | SLQSSLSLPS |
| T257106_3: | mitrans | lncRNA | lincRNA | LFLLTSLSCHLVKKDLFASSSAMIVNF |
| 191430385-191497326:+ | | | | LRPSQTYETLPCEEGLACFPFHHGCKF |
| | | | | PEAFPAMQNCESIKPFSFINYPISGMS |
| | | | | S |
| T257107_3: | mitrans | lncRNA | lincRNA | LFLLTSLSCHLVKKDLFASSSAMIVNF |
| 191430385-191501910:+ | | | | LRPSQTYETLPCEEGLACFPFHHGCKF |
| | | | | PEAFPAMQNYQTFYP |
| T269973_4: | mitrans | lncRNA | lincRNA | MEVCSTNHTEMQSAPNWPQPKGDKFNS |
| 115068324-115068836:+ | | | | CGSELASLERRGRHSMGTRQKLPVSYS |
| | | | | RQLGPCVPAQQGGEGGGEPLLTGPSGK |
| | | | | RKQRAVSGIGGFLVSLTSNMKPRTLTV |
| | | | | SVTVLKGGVSGASSF |
| T280212_5: | mitrans | lncRNA | lincRNA | LLRVWGPAEPTPIQSSRWLTSGTHSPG |
| 28078042-28084058:+ | | | | SRLRLSLLTSGQAEGAGSGLGQPRKGL |
| | | | | PQCSRRLKGSPSAARVGAEAEEAPRAS |
| | | | | EGCEGRQQAVTSQYRTGQELENHQTAR |
| | | | | VKGAITLSWAAHQAMGGDMELDCGSEE |
| | | | | WQPFWGLRLWDALSQSCCKSPPASHWH |
| | | | | QAATPHNSSRAGIAQEPWARVKQGSRT |
| | | | | ETALT |
| T312199_6: | mitrans | lncRNA | lincRNA | VGGILPPVDITMTLLNWKLRLPLGHFG |
| 138014181-138019867:+ | | | | LLMPGSTGQGGSYSVGCMADLTTKGKL |
| | | | | DYCSTMEILSGLFHGSGTTSSFPTS |
| T333716_7: | mitrans | lncRNA | lincRNA | LVSSLSACCADFSLASPHNHVLSLIPD |
| 134396289-134453187:+ | | | | HQCPSSSTCRSRRLAPSKWVSAGSHFI |
| | | | | CTLLIKQVAPSAEQVLSRLWLKLLLLK |
| | | | | E |
| T349874_8: | mitrans | lncRNA | lincRNA | MILSKRQMNGDPGRPFL |
| 116594833-116594884:+ | | | | |
| ENST00000616578.1_1_13: | RP11- | lncRNA | lincRNA | LVRKKMSTS |
| 55637129-55680923:+ | 78L16.1 | | | |
| T035001_1: | mitrans | lncRNA | lincRNA | MGPCWGESGENNRRGRPGNAGMEGA |
| 248469615-248469690:+ | | | | |
| T333717_7: | mitrans | lncRNA | lincRNA | VIESFPRPPSAMLPVQPAKPSSALFRT |
| 134398878-134453113:+ | | | | ISALLLPPAAPGDWPHQSGSVQALISS |
| | | | | APY |
| T333717_7: | mitrans | lncRNA | lincRNA | LDFSSNTSSSLVSSLSACCADFSLASP |
| 134396259-134398879:+ | | | | HNHAWKMCLLPLYLLP |
| T257122_3: | mitrans | lncRNA | lincRNA | MSERKHFKHYIKFFICFFCVALMMTLV |
| 191430341-191477334:+ | | | | FS |
| T257106_3: | mitrans | lncRNA | lincRNA | MSERKHFKHYIKFFICFFSLLFPAIL |
| 191430341-191495054:+ | | | | |
| T192659_2: | mitrans | lncRNA | lincRNA | RVSKVEKFSAERRLE |
| 81709163-81709211:+ | | | | |
| T257107_3: | mitrans | lncRNA | lincRNA | LGALGARTPGNILATTKGHPK |
| 191377069-191430242:+ | | | | |
| T314020_6: | mitrans | lncRNA | lincRNA | LCDLLCDMNYCLRLGT |
| 155827068-155827119:+ | | | | |
| ENST00000557481.6_1_15: | LINC01579 | lncRNA | lincRNA | LFCHSIKLFSILLSLHLSAIPHLPGRR |
| 94442527-94443685:− | | | | TRTQDPPGGLTFRRGASWTA |
| ENST00000557715.1_1_15: | LINC01579 | lncRNA | lincRNA | LAPGAASPTAAAGLTFRRGASWTA |
| 94442527-94443301:− | | | | |
| ENST00000247452.3_1_X: | MAGEC2 | Out-of- | Out-of- | RGICWEGALRLWGA |
| 141290961-141291006:− | | Frame | Frame | |
| ENST00000593011.5_1_1: | FLG-AS1 | lnCRNA | Antisense | MGGWHGSGLRGG |
| 152140699-152140738:+ | | | | |
| ENST00000437267.2_1: | RP11- | lncRNA | lincRNA | MITKTQVQRQSSYIVPASDPPW |
| 143186450-143186519:− | 782C8.2 | | | |
| ENST00000233638.7_1_2: | TLX2 | 5′ | 5′ | MLLFRGPRRPALASPAGP |
| 74741778-74741835:+ | | uORF | uORF | |
| ENST00000409910.1_1_2: | AC112721.2 | lncRNA | lincRNA | IRHPEMDMPNVKPSRPFVAAASGKRTS |
| 238337821-238343336:− | | | | PLLINHESVSAFANGFLVSSSVG |
| T257119_3: | mitrans | lncRNA | lincRNA | MSERKHFKHYIKFFICFFYKLRPSLK |
| 191430341-191444036:+ | | | | |
| ENST00000450877.5_1_7: | IMMP2L | Out-of- | Out-of- | RTRRAVLNVIL |
| 111031942-111031978:− | | Frame | Frame | |
| ENST00000632314.1_1_10: | FRMD4A | lncRNA | ncRNA | IPSVSKTDEPRVAPDNVLLAECALISG |
| 13726607-13726712:− | | | Processed | AESPRVF |
| | | | Transcript | |
| ENST00000632314.1_1_10: | FRMD4A | lncRNA | ncRNA | VSPGSQTMFIASGSSLQRFPNAPENDF |
| 13726312-13726411:− | | | Processed | LTWRE |
| | | | Transcript | |
| ENST00000632314.1_1_10: | FRMD4A | lncRNA | ncRNA | MPPKMTF |
| 13726326-13726350:− | | | Processed | |
| | | | Transcript | |
| ENST00000567333.1_1_16: | RARRES2P6 | Pseudogene | Pseudogene | MQWPSGRTVWKARWTRPSQLEHS |
| 34489847-34489919:− | | | | |

TABLE 3C
Riboseq-based CLL

| ORF_ID | geneName | plotType | mergeType | sequence |
|---|---|---|---|---|
| ENST00000583608.1_1_17: | ARHGAP44 | 5′ | 5′ | MREASVQSPGDMAGVEPWLNLALLIFF |
| 12859099-12859180:+ | | uORF | uORF | |
| ENST00000423473.6_1_17: | CCDC144NL- | lncRNA | Antisense | MKLSWSTIWEFQERLFQSWAPEDNVVL |
| 20899806-20901188:+ | AS1 | | | RNLQTSVKELTRKHWDLPPPVQAVCTP |
| | | | | EPGPTAPRVP |
| ENST00000611838.1_1_6: | XXbac- | lncRNA | Antisense | LPAWRSYSTPQDFKREIPPGLSHREES |
| 32223085-32317317:+ | BPG154L12.5 | | | DFLRLFQSGLEIALFMSS |
| T013936_1: | mitrans | lncRNA | lincRNA | LSVTGMDGVHMIAGSLRLS |
| 83945334-83945391:− | | | | |
| T162485_18: | mitrans | lncRNA | lincRNA | LCQRHTLIDPLSRTRAANLTGDLTYLN |
| 52673962-52762449:− | | | | HLRKQEGWEREVTVQLSLNSDQMFNQM |
| | | | | HEEWAKCKPQLNPSGRGVKVLVILNYV |
| | | | | FSCLESGSYNSLQGKLRASL |
| T162488_18: | mitrans | lncRNA | lincRNA | LRKQEGWEREVTVQLSLNSDQMFNQMH |
| 52746328-52762365:− | | | | EEWAKCKPQLNPSGRGVKVLVILNYGN |
| | | | | PDTDMRRGRRLCEEEGSGWSDDSPSLG |
| | | | | RPVTGSKAPETRGRNMEPIPSQASGGT |
| | | | | NPDEP |
| T314055_6: | mitrans | lncRNA | lincRNA | LECCLEKTTERVILHQ |
| 156615822-156615870:− | | | | |
| ENST00000559212.1_1_15: | PCAT29 | lncRNA | lincRNA | VWPVDDAIKLRCEAEVRQRLPSFCLAQ |
| 69973631-69988065:+ | | | | FSVREQRWEMRGPLPQCFCIQAPALLK |
| | | | | VAPTATLHTAARRTLKYRSF |
| T169145_19: | mitrans | Other | TUCP | LGSSLRGPSLCARPPRPGPQGRKHLVW |
| 13215208-13215725:+ | | | | SGCTGRLVPVQLPPLWPLESWAKETRR |
| | | | | RGGSGAKTRGGYRAGAPHYAQARGLES |
| | | | | AEDSTLQPGRGERSPEEQIPVMQLYGP |
| | | | | IHKPQAIGRR |
| ENST00000531807.5_1_11: | RRAS2 | 5′ Overlap | 5′ Overlap | MLYKCMRMTDTKFRILITSWCWGRREG |
| 14363366-14363504:− | | uORF | uORF | NIIEEACKSLQSYYKCFVS |
| ENST00000522554.1_1_4: | RP11- | lncRNA | lincRNA | ILKRQEPQILEYCKMTFQIDEPWTPSW |
| 185546006-185546117:− | 242J7.1 | | | IDTEMKRRD |
| T312467_6: | mitrans | lncRNA | lincRNA | MALRDTRLEALGADQTHSKEEWWLKTA |
| 139750544-139750634:+ | | | | CS |
| ENST00000596488.1_1_19: | AC010524.4 | lncRNA | Antisense | MSRRQEMRISEPAGRQGASLSCWTMKG |
| 49859474-49860832:+ | | | | EKHTEDESHLGSTRTQMSIKELVNTTD |
| | | | | PPGTHPIQASFPSTMLSLWLLWV |

TABLE 3D
Riboseq-based GBM

| geneName | plotType | mergeType | sequence |
|---|---|---|---|
| RPH3A | 5′ | 5′ | LGSLAPPLSF |
| | uORF | uORF | |
| RP11- | lncRNA | lincRNA | VPTLLCLSLEKPVRT |
| 266O8.1 | | | RMGKVSGSWCS |
| RP11- | lncRNA | lincRNA | MEEMAGTKGQRNKKP |
| 266O8.1 | | | |
| mitrans | Other | TUCP | LKSGKIRIIAAASAP |
| | | | GRGGSGLWLAGEGCT |
| | | | GRLGEDLAGARE |
| mitrans | lncRNA | lincRNA | MHERWEVGARASCLM |
| | | | LISEE |
| PCDH19 | 5′ | 5′ | LLAHTPHSHSPHHTL |
| | uORF | uORF | TLPLSLTHSHTARDR |
| | | | QRKRDRPPECASARE |
| | | | EGSPTQRGQRLSGKM |
| | | | KSRRSCK |
| LINC01965 | lncRNA | lincRNA | VGKLERLNVLWSSQR |
| | | | FGSGRRKTKRSIVGV |
| | | | LNRLSQDPQDQWSVA |
| | | | VLAINLCGQWLLCQG |
| | | | FAWAYWASSAHSACQ |
| | | | AVLSTHYMPGSHVCQ |
| | | | GLVRGGAVRGV |
| SOX2- | Other | Sense | LGLSGTLNAMKAVCV |
| OT | | Overlapping | ERLKCISKPGLIRGT |
| | | | RKNRPMCIL |
| SOX2- | Other | Sense | MHFKARPDSGYKEEQ |
| OT | | Overlapping | TYVHFVGRKL |
| RP11- | lncRNA | lincRNA | MSPESVQPDDA |
| 436K8.1 | | | |
| DMRTA2 | 5′ | 5′ | LKGGLDCAAF |
| | uORF | uORF | |
| SOX2- | Other | Sense | MEKGEGERNGGNLDN |
| OT | | Overlapping | TLK |
| PNMA2 | lncRNA | ncRNA | MWAVSAMNCVGPSRG |
| | | Processed | KVCRNLVCVCSS |
| | | Transcript | |
| ZEB1 | 5′ | 5′ | MTCYSKEWSIGYCNF |
| | uORF | uORF | NFLF |
| PRPH | lncRNA | ncRNA | MARKEGGAWKGDEGG |
| | | Processed | PI |
| | | Transcript | |

As MHC I immunopeptidomes are likely incomplete, Applicants also used the Ribo-seq data to identify additional nuORFs that are translated in a cancer-specific manner (FIG. 35A, 35I), and lowly expressed (TPM <1, excluding testis) across healthy tissues in GTEx (FIG. 35I, Table 2A-2B). Encouragingly, many of the selected nuORFs had significantly higher expression in the matched cancer type compared to healthy tissues and other TCGA samples (FIG. 35I, 35J). In particular, 13 nuORFs were strongly upregulated in CLL compared to GTEx and other TCGA samples (FIG. 35K). For example, Applicants found a CLL-specific 5′uORF in the ARHGAP44 gene which has been shown to be upregulated in CLL patients up to 10 years prior to diagnosis (32). The ARHGAP44 5′ uORF is translated from a 5′UTR of a CLL-specific ARHGAP44 transcript isoform, not expressed in healthy B cells and different from the isoform expressed in other tissues, such as melanoma (FIG. 35L). Another CLL-specific 5′ouORF detected in the RRAS2 gene is upregulated in CLL patients with 13q deletions (33). Given the low frequency of somatic mutations in CLL (6), these CLL-specific nuORFs could provide new targets for therapy.

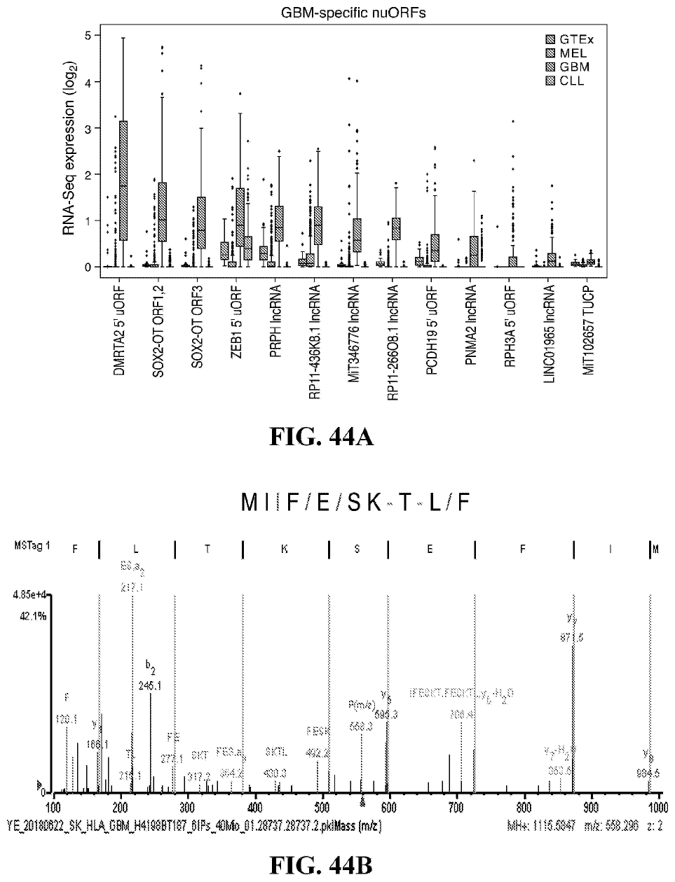

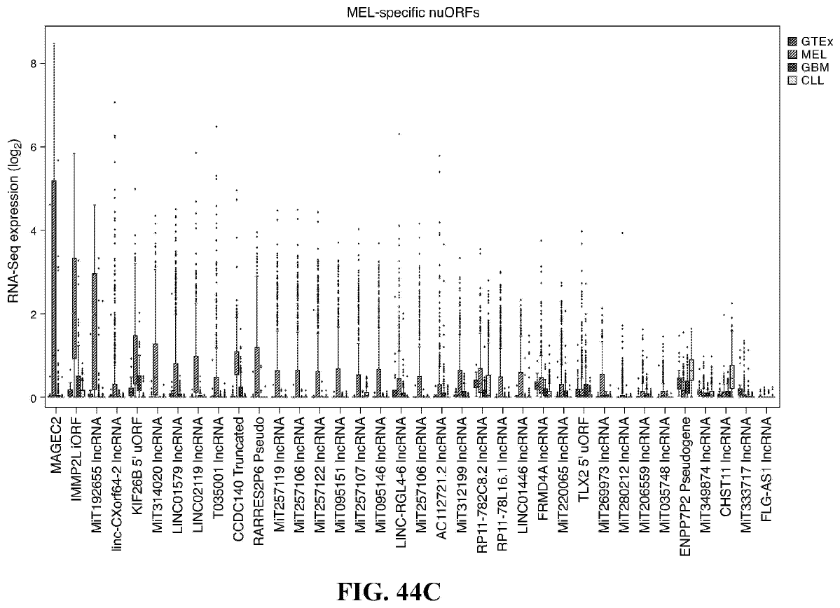

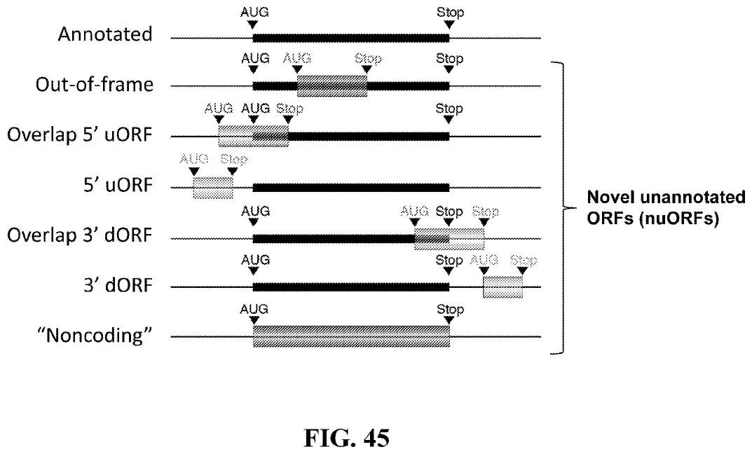

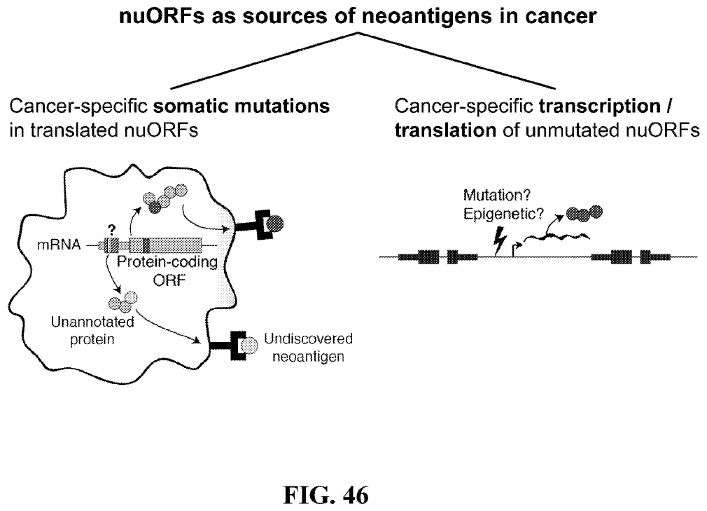

Applicants similarly identified several GBM and melanoma-specific nuORFs (FIG. 44A, 44C). In GBM, several nuORFs were translated from the SOX2-OT “noncoding” transcript (FIG. 44A) and a peptide (MIFESKTLF) derived from one of the SOX2-OT nuORFs was detected in the MHC I immunopeptidome of one of the GBM samples (FIG. 44B). SOX2-OT, annotated as a lncRNA, is frequently upregulated in GBM patients, and is essential for GBM tumorigenesis (34). Given that SOX2-OT harbors several nuORFs specifically translated in GBM, its role in GBM pathogenesis and potential immunogenicity should be explored.

In summary, nuORFs are abundant, translated, and contribute antigens to MHC I presentation in cancer cells. NuORFs can harbor somatic mutations, and can be translated in a cancer-specific manner, providing a potential source of immunogenic targets. NuORFdb can be used to identify nuORFs even in sample types not profiled by Ribo-seq. The preponderance of translated nuORFs of various types opens the question of whether nuORFs have a biological function in cells. A further question is whether their primary function may be to serve as cancer-specific signals to the immune system. The data suggest that nuORFs should be considered when exploring the immunopeptidome, and that they may have potential utility in selecting candidates for immunotherapeutic targeting.

### Methods

### Cell Cultures

A375 cells were cultured in DMEM media (Gibco), supplemented with 5% fetal bovine serum (FBS). HCT116 cells were cultured in McCoy's 5A Medium (Thermo Fisher Scientific), supplemented with 5% FBS.

### Generation of HLA Mono-Allelic B721.221 Cells

The HLA mono-allelic cell lines were generated as previously described (1, 2). Briefly, single HLA allele-expressing cDNA vectors in a pcDNA-3 backbone were ordered from GenScript™. The HLA class I deficient B721.221 cell line was transfected with the HLA allele expression vectors using lipofectamine, as described previously (3). Cell lines with stable surface HLA expression were generated first through selection using 800m/m1 G418 (Thermo Fisher Scientific), followed by enrichment of HLA positive cells through up to 2 serial rounds of fluorescence-activated cell sorting (FACS) and isolation using a pan-HLA antibody (W6/32; Santa Cruz) on a FACSAria II instrument (BD Biosciences).

### Primary Human Cells and Generation of Cancer Cell Lines

All human tissues were obtained through DFCI or Partners Healthcare approved IRB protocols. Conditions for growth and in vitro propagation of melanoma and GBM tumor cell lines were described previously (4, 5). PBMCs from fresh healthy donor whole blood were isolated using Ficoll density gradient medium. CD19⁺ B cells were isolated using EasySep Human CD19 Positive Selection Kit, obtaining between 25 and 54 million B cells per donor. For fresh CLL samples, PBMCs were isolated using Ficoll density gradient medium, enriched for CD19 positive CLL tumor cells and were used in IP/MS analysis and Ribo-seq. For cryopreserved CLL samples, live cells were isolated with an OptiPrep density gradient medium.

Tumor specimens from patients with clear cell renal cell carcinoma (ccRCC) were collected following informed consent for enrollment on a tissue collection research protocol approved by the Dana-Farber/Harvard Cancer Center Institutional Review Board (IRB). Surgically resected ccRCC tumor tissue was mechanically dissociated with scalpels, and then enzymatically dissociated using a mixture of collagenase D (Roche), Dispase (STEMCELL Technologies), and DNase I (New England BioLabs) at room temperature, and filtered through a 100 micron cell strainer using the sterile plunger of a syringe. Red blood cells were lysed using ammonium-chloride-potassium buffer (Gibco). The cell suspension was stained for viability (Zombie Aqua; BioLegend), anti-CD45 (BV605; BD Biosciences), and anti-carbonic anhydrase IX (PE; R&D Systems). Viable, CD45⁻, CAIX⁺ tumor cells were isolated by FACS (BD FACSAria II cell sorter; BD Biosciences). Cells were cultured in a specialized growth medium consisting of OptiMEM GlutaMax media (Gibco), 5% fetal bovine serum, 1 mM sodium pyruvate (Gibco), 100 units/mL penicillin and streptomycin, 50 micrograms/mL gentamicin, 5 micrograms/mL insulin (Sigma), and 5 ng/mL epidermal growth factor (Sigma). Following successive passages, CAIX expression was confirmed by flow cytometry (anti-CAIX, PE-conjugated; R&D Systems) and by immunohistochemical analysis of a cell pellet.

Ovarian cancer patient-derived cells were propagated within a xenograft model, which was generated by serial passaging of tumor cells from a patient with advanced ovarian cancer. These cells originated from solid tumor that were injected orthotopically in the abdominal cavity in NOD-SCID mice (8-week old, Jackson labs). Tumor growth was monitored weekly by observing mice for signs of abdominal distension. Cells were harvested 4 months after initial injection and banked for future experiments.

### Ribosome Profiling

Ribosome profiling was performed according to the manufacturer's protocol (TruSeq Ribo Profile—RPHMR12126, Illumina, discontinued), with the following modifications. For adherent cell lines (melanoma, primary melanocytes, HCT116, A375), culture media was removed, cells were washed with ice-cold PBS containing cycloheximide (0.1 mg/ml) and lysed in the Lysis Buffer according to the Illumina protocol. For suspension cell lines and primary blood samples, cells were spun 1,000 rpm for 5 minutes, washed once with ice-cold PBS containing cycloheximide (0.1 mg/ml) and lysed in the Lysis Buffer. To perform Ribo-seq on small samples, such as primary B cells and melanocytes, cells were lysed in 200 μl of lysis buffer, such that the entire lysate could be used in library preparation. Ribosomes containing ribosome-protected mRNA fragments (RPFs) were enriched using MicroSpin S-400 columns (GE Healthcare, catalog #27-5140-01). Ribo-zero rRNA Removal Kit (Illumina, MRZH11124, discontinued) was used to deplete rRNA from RPFs. The entire RPF sample was loaded on a 15% urea-polyacrylamide gel. Samples were eluted from the gel overnight at 4° C. Subsequently, end repair, adapter ligation and reverse transcription were carried out according to the manufacturer's protocol. For the cDNA gel purification, the reverse transcription reaction was loaded on a 10% urea-polyacrylamide gel. The samples were eluted from the gel overnight at room temperature. Subsequently, RPFs were circularized and 5 l of circDNA was used for library amplification. The number of amplification cycles was determined based on the observed sample quality and expected yield, but usually ranged between 8 and 10 cycles. Following amplification, the library was gel-purified using 4% E-Gel EX Agarose Gel (ThermoFisher G401004) and Zymoclean Gel DNA Recovery Kit (Zymo Research D4007), with 4 volumes of ADB buffer to accommodate 4% agarose gel. The resulting libraries were analyzed for quality using Agilent Bioanalyzer 2100 and sequenced for 51 cycles on the Illumina NextSeq platform, using NextSeq 500 high output kit, V2, 75 cycles.

### Ribo-Seq Data Pre-Processing

To process RPF sequencing reads, Illumina adapters were removed using fastx_clipper from the FASTX-Toolkit. Ribosomal RNA and tRNA were removed using Bowtie version 1.0.0 (6). Remaining reads were aligned to the genome (hg19/GRCh37) and transcriptome using STAR version 2.5.3a (7) (—alignIntronMin 20—alignIntronMax 100000—outFilterMismatchNmax 1—outFilterType BySJout—outFilterMismatchNoverLmax 0.04—twopassMode Basic). For the transcriptome annotation, a combination of GENCODE v26lift37 transcriptome annotation was combined with transcripts annotated as tstatus “unannotated” from MiTranscriptome annotation (8). To determine the RPF library quality, trinucleotide codon periodicity was plotted using RibORF readDist script (9) against annotated protein-coding ORFs (GENCODE v26lift37). Only samples and read lengths that showed clear trinucleotide periodicity were used for subsequent ORF predictions.

### Hierarchical Prediction of Translated Open Reading Frames Across Tissues

In order to maximize the detection of translated ORF and overcome noise from overlapping ORFs expressed in different tissues, Applicants performed hierarchical ORF predictions using RibORF (9) and PRICE (10), as follows.

For RibORF, only read lengths that showed clear trinucleotide periodicity were used for ORF predictions. RibORF offsetCorrect script was used to correct the RPF offsets for each read length. As input, for the transcriptome reference, GENCODE v26lift37 transcriptome annotation was combined with transcripts annotated as tstatus “unannotated” from MiTranscriptome annotation (8). From this custom transcriptome reference, all possible ORFs with NTG start codons and TAA/TGA/TAG stop codons were identified using Rp-Bp prepare-rpbp-genome script (11). For the GENCODE ORF search, Rp-Bp reported the following ORF types based on the annotation of the transcript and the location of the ORF within the transcript:
canonical: identical to a protein-coding ORF annotated in the GENCODE reference.
canonical_extended: Predicted start is 5′ extended relative to a protein-coding ORF annotated in the GENCODE reference.
canonical_truncated: Predicted start codon is 3′ downstream of the annotated start codon in the GENCODE reference.
five_prime: ORF entirely contained in the 5′ UTR of a protein-coding transcript.
five_prime_overlap: ORF with a start codon in the 5′ UTR of a protein-coding transcript, and a stop codon within an annotated ORF, out-of-frame relative to the annotated ORF.
three_prime: ORF entirely contained in the 3′ UTR of a protein-coding transcript.
three_prime_overlap: ORF with a start codon within an annotated ORF, and the stop codon in the 3′ UTR, out-of-frame relative to the annotated ORF.
within: entirely contained within, but out of-of-frame relative to an annotated ORF.
noncoding
suspect

Those ORFs annotated as noncoding or suspect by Rp-Bp were re-annotated based on the metadata column in the GENCODE GTF. The ORFs derived from transcripts containing ‘line’ or ‘pseudo’ in the metadata column were annotated as noncoding_lincRNA or noncoding_pseudogene respectively. Otherwise, they were re-annotated as noncoding_other. For the MiTranscriptome transcripts, Rp-Bp reported all ORFs as either noncoding or suspect. Subsequently, the ORF types were re-annotated as noncoding mi_lincRNA or noncoding_mi_tucp based on the transcript type annotated in the MiTranscriptome GTF as either tcat “lncrna” or tcat “tucp” respectively. After running RibORF, ORFs with a score >0.7 were retained. If multiple ORFs on the same transcript shared a common stop codon, the longest ORF was selected.

Hierarchical ORF prediction using RibORF: Offset-corrected SAM files across samples were combined at each branch and at the root (FIG. 36A). For the ORFs predicted at the root, Applicants retained predicted ORFs with at least 2 reads in-frame and a RibORF score >0.7. For ORFs predicted at the branches and leaves (FIG. 36A) Applicants retained predicted ORFs with at least 2 reads and score >0.9, or at least 250 reads and score >0.7.

For PRICE, Applicants ran the PRICE pipeline (10) on unprocessed fastq.gz files of the samples that had clear tri-nucleotide periodicity (as determined by RibORF above) with the same reference transcriptome as for RibORF. The pipeline handled adapter trimming, rRNA and tRNA removal, offset correction and ORF prediction. Unique .cit files were generated for each sample. For the hierarchical ORF prediction using PRICE, gedi MergeClT was used to merge samples by tissue type at each branch and at the root. gedi Price-fdr 1 was used to predict translated ORFs. The PRICE ORF annotation types (10) and https://github.com/erhard-lab/gedi/wiki/Price include the following:
CDS: ORF is exactly as in the annotation
Ext: ORF contains a CDS, ending at its stop codon
Trunc: ORF is contained in a CDS, ending at its stop codon
Variant: ORF ends at a CDS stop codon, but is neither Ext nor Trunc
uoORF: ORF starts in 5′-UTR, ends within a CDS
uORF: ORF starts and ends in 5′-UTR
iORF: ORF is contained within a CDS
dORF: ORF ends in 3′-UTR
ncRNA: ORF is located on non-coding transcript
intronic: ORF is located in an intron
orphan: Everything else
   Generating nuORFdb v1.0

FASTA files of ORFs predicted across tissues by RibORF and PRICE were combined, and those ORFs entirely contained within other predicted ORFs at the protein level were removed. Predicted ORFs over 21 nucleotides long were retained for the downstream analysis, and translated in the single frame determined from Ribo-seq periodicity. After merging the predictions from RibORF and PRICE, the nuORFdb contains the ORF types from both prediction tools, as described above. To improve annotations, for nuORFs in categories ncRNA, noncoding_other, orphan, and Variant, Applicants identified their transcript_type annotated in the GENCODE GTF metadata and generated the nuORF Refined type (Table S9). In order to unify the different terms for the same concept Applicants subsequently merged the refined ORF types according to the specifications of biotypes in Ensembl (https:useast.ensembl.org/info/genome/genebuild/biotypes.html), generating an ORF type mapping table (Table S9), where MergedType is used in FIG. 38A, and PlotType is used in the rest of the figures, also shown in FIG. 32E.

### HLA-Peptide Immunoprecipitation, Sequencing by Tandem Mass Spectrometry, Peptide Identification

Soluble lysates from up to 50 million HLA expressing B721.221 cells or 0.1 to 0.2 g cancer cells were immunoprecipitated with W6/32 antibody (sc-32235, Santa Cruz) as described previously (1, 2). 10 mM iodoacetamide was added to the lysis buffer to alkylate cysteines for 71 alleles and 12 tumor samples. Peptides of up to three IPs were combined, acid eluted either on StageTips or SepPak cartridges (12), and analyzed in technical duplicates using LC-MS/MS. Peptides were resuspended in 3% ACN, 5% FA and loaded onto an analytical column (20-30 cm, 1.9 μm C18 Reprosil beads (Dr. Maisch HPLC GmbH), packed in-house PicoFrit 75 μm inner diameter, 10 μm emitter (New Objective)). Peptides were eluted with a linear gradient (EasyNanoLC 1000 or 1200, ThermoFisher Scientific) ranging from 6-30% Buffer B (either 0.1% FA or 0.5% AcOH and 80% or 90% ACN) over 84 min, 30-90% B over 9 min and held at 90% Buffer B for 5 min at 200 nl/min. During data dependent acquisition, peptides were analyzed on a QExactive Plus (QE+), QExactive HF (QE-HF) or Fusion Lumos (ThermoFisher Scientific). Full scan MS was acquired at a resolution of 70,000 (QE+) or 60,000 (QE-HF and Lumos) from 300-1,800 m/z or 300-1,700 m/z (Lumos). AGC target was set to 1e6 and 5 msec max injection time for QE type instruments and 4e5 and 50 ms for Lumos. The top 10 (Lumos, QE+), 12 (QE+), 15 (QE-HF) precursors per cycle were subjected to HCD fragmentation at resolution 17,500 (QE+) or 15,000 (QE-HF, Lumos). The isolation width was set to 1.7 m/z with a 0.3 m/z offset for QE and 1.0 m.z and no offset for Lumos, the collision energy was set to optimal for the instrument used ranging from 25 to 30 NCE, AGC target was 5E4 and max fill time 120 ms (QE+ and Lumos) or 100 ms (QE-HF). For Lumos measurements, precursors of 800-1700 m/z were also subjected to fragmentation if they were singly charged. Dynamic exclusion was enabled with a duration of 15 sec (QE+), 10 secs (QE-HF) or 5 sec (Lumos).

### HLA Peptide Identification Using Spectrum Mill

Mass spectra were interpreted using the Spectrum Mill software package v6.1 pre-Release (Agilent Technologies, Santa Clara, Calif.). MS/MS spectra were excluded from searching if they did not have a precursor MH+ in the range of 600-4000, had a precursor charge >5, or had a minimum of <5 detected peaks. Merging of similar spectra with the same precursor m/z acquired in the same chromatographic peak was disabled. Prior to searches, all MS/MS spectra had to pass the spectral quality filter with a sequence tag length >2 (i.e., minimum of 4 masses separated by the in-chain masses of 3 amino acids).

MS/MS spectra were searched against the 323,848 protein sequences in nuORFdb v1.0 appended to a base reference proteome containing all UCSC Genome Browser genes with hg19 annotation of the genome and its non-redundant protein coding transcripts (52,788 entries) as well as 264 common laboratory contaminants, including proteins present in cell culture media and immunoprecipitation reagents. MS/MS data from patient derived cell lines was analyzed in the same way, except that the sequence database was revised with further inclusion of patient-specific somatic mutations.

MS/MS search parameters included: no-enzyme specificity; fixed modification: cysteinylation of cysteine; variable modifications: carbamidomethylation of cysteine, oxidation of methionine, and pyroglutamic acid at peptide N-terminal glutamine; precursor mass tolerance of ±10 ppm; product mass tolerance of ±10 ppm, and a minimum matched peak intensity of 30%. Variable modification of carbamidomethylation of cysteine was only used for HLA alleles that included an alkylation step (Table S7). Peptide spectrum matches (PSMs) for individual spectra were automatically designated as confidently assigned using the Spectrum Mill auto-validation module to apply target-decoy based FDR estimation at the PSM level of <1% FDR. Peptide auto-validation was done separately for each HLA allele with an auto thresholds strategy to optimize score and delta Rank1-Rank2 score thresholds separately for each precursor charge state (1 through 4) across all LC-MS/MS runs for an HLA allele. Score threshold determination also required that peptides had a minimum sequence length of 7, and PSMs had a minimum backbone cleavage score (BCS) of 5. BCS is a peptide sequence coverage metric and the BCS threshold enforces a uniformly higher minimum sequence coverage for each PSM, at least 4 or 5 residues of unambiguous sequence. The BCS score is a sum after assigning a 1 or 0 between each pair of adjacent AA's in the sequence (max score is peptide length-1). To receive a score, cleavage of the peptide backbone must be supported by the presence of a primary ion type for HCD: b, y, or internal ion C-terminus (i.e., if the internal ion is for the sequence PWN then BCS is credited only for the backbone bond after the N). The BCS metric serves to decrease false-positives associated with spectra having fragmentation in a limited portion of the peptide that yields multiple ion types. PSMs were consolidated to the peptide level to generate lists of confidently observed peptides for each allele using the Spectrum Mill Protein/Peptide summary module's Peptide-Distinct mode with filtering distinct peptides set to case sensitive. A distinct peptide was the single highest scoring PSM of a peptide detected for each allele. MS/MS spectra for a particular peptide may have been recorded multiple times (e.g., as different precursor charge states, from replicate IPs, from replicate LC-MS/MS injections). Different modification states observed for a peptide were each reported when containing amino acids configured to allow variable modification; a lowercase letter indicates the variable modification (C-cysteinylated, c-carbamidomethylated).

In cases where a spectrum could be matched to multiple proteins due to shared peptide sequences, the Spectrum Mill output was revised so that the primary protein assignment for a spectrum was determined using the following decision tree, in order of diminishing assignment priority: Contaminants→annotated proteins→nuORFs. In cases where a spectrum could be matched to multiple annotated proteins, priority was given to the more highly translated one based on Ribo-seq TPM. In cases where a spectrum could be matched to multiple nuORFs, priority was given to the more highly translated based on Ribo-seq TPM. In case of equal Ribo-seq TPM, the primary assignment was randomly selected.

FDR Filtering of nuORF-Derived Peptides

Applying the same aggregate FDR threshold to the combination of peptides observed for both annotated ORFs and nuORFs resulted in a much higher FDR for nuORFs (4.6%) than for annotated ORFs (1%), which was as high as 14% for certain nuORF categories, such as 3′ overlapping dORFs (FIG. 37C, 37D). Applicants therefore introduced more stringent filtering for nuORF peptides (FIG. 37E, 37F), to retain only the 6,501 which achieved <1% peptide-level FDR (FIG. 37A-37D, 37G). Spectra were removed based on fixed thresholds for 4 Spectrum Mill MS/MS scoring metrics: score, backbone cleavage score (BCS), BCS %, and percent scored peak intensity, defined as follows:
Score: the primary score based on assignment of the full range of ion types (y, b, a, internal and neutral losses of NH₃ and H₂O) to peaks in a spectrum.
Backbone cleavage score (BCS): absolute peptide sequence coverage metric described above
BCS %: BCS normalized for peptide length, 100*BCS/(sequence length−1)
Percent scored peak intensity: Percent of product ion intensity in an MS/MS (after peak detection) that is matched to a scored ion type.

NuORFs across all 92 alleles were binned by ORF type as described in Table 2A-2B. FDRType column and integer thresholds were calculated per bin to maximize retained spectra with an FDR less than 1% (FIG. 37C, 37D). Maximal thresholds were calculated using a grid search of integer threshold values encompassing the empirically observed values. Specifically, Applicants identified the combination of lowest values across the 4 scoring metrics that resulted in FDR <1% for each ORF type bin.

Peptide Spectrum Matching with Proposed Splice Peptides

For 9 of our previously published monoallelic datasets (A02:03, A02:04, A02:07, A03:01, A24:02, A31:01, A68:02, B44:02, B51:01) (3) that have been proposed to contain proteasomal spliced peptides (13) Applicants reanalyzed the data to examine if nuORF derived peptides could be better explanations for the spectra matched to proposed splice peptides. Since Faridi et al 2018 did not make detailed data publicly available that indicated which spectra were matched to individual spliced peptides for the datasets, Applicants took the proposed spliced peptides in their supplemental tables, and appended them to the nuORFdb/Reference proteome database and repeated the analysis of the spectra for these 9 alleles using the process described above. Results where a nuORF peptide and one or more proposed spliced peptides yield consistent tie-score matches to the same spectra are provided in Table S5.

### Peptide Hydrophobicity Index Calculation

Hydrophobicity index was predicted using SSRCalc (14), http://hs2.proteome.ca/SSRCalc/SSRCalcQ.html). Modification of cysteine was checked for alleles B5601 and A7401. For A0201 and C0304 free Cysteine was specified.

### Whole Proteome Analysis and Interpretation

Protein expression of the B721.221 and GBM H4152-BT145 cell lines was assessed as described previously (15). Briefly, cell pellets of B721.221 cells expressing A*03:01, B*55:01 and C*07:01, as well as pellets of GBM6 with and without IFNγ treatment were lysed in 8M Urea and digested to peptides using LysC and Trypsin (Promega). B721 analysis was performed label free with a 1:1:1 mix using 100 μg each of the three monoallelic cell lines. For GBM, 100 μg peptides were labeled with TMT6 reagents (Thermo Fisher) 126 (untreated) and 127 (IFNγ) and then pooled for subsequent fractionation and analysis. Pooled peptides were separated into 24 fractions using offline high pH reversed phase fractionation. 1 μg per fraction was loaded onto an analytical column (20-30 cm, 1.9 m C18 Reprosil beads (Dr. Maisch HPLC GmbH), packed in-house PicoFrit 75 M inner diameter, 10 M emitter (New Objective)). Peptides were eluted with a linear gradient (EasyNanoLC 1000 or 1200, Thermo Scientific) ranging from 6-30% Buffer B (either 0.1% FA or 0.5% AcOH and 80% or 90% ACN) over 84 min, 30-90% B over 9 min and held at 90% Buffer B for 5 min at 200 nl/min. During data dependent acquisition, peptides were analyzed on a Fusion Lumos (Thermo Scientific). Full scan MS was acquired at a 60,000 from 300-1,800 m/z. AGC target was set to 4e5 and 50 ms. The top 20 precursors per cycle were subjected to HCD fragmentation at 15,000 resolution with an isolation width of 0.7 m/z, 30 NCE, 3e4 AGC target and 50 ms max injection time. For TMT experiments, resolution was set to 60,000 and 34 NCE. Dynamic exclusion was enabled with a duration of 45 sec.

Spectra were searched using Spectrum Mill against the same database as the one used for the MHC I IP/MS spectra analysis (described above), specifying Trypsin/allow P as digestion enzyme, allowing 4 missed cleavages. Carbamidomethylation of cysteine was set as a fixed modification. For the GBM dataset TMT labeling was required at lysine, but peptide N-termini were allowed to be either labeled or unlabeled. Allowed variable modifications were acetylation at the protein N-terminus, oxidized methionine, pyroglutamic acid, deamidated asparagine and pyrocarbamidomethyl cysteine. Match tolerances were set to 20 ppm on MS1 and MS2 level. PSMs score thresholding used the Spectrum Mill auto-validation module to apply target-decoy based FDR in 2 steps: at the peptide spectrum match (PSM) level and the protein level. In step 1 PSM-level autovalidation was done first using an auto-thresholds strategy with a minimum sequence length of 8; automatic variable range precursor mass filtering; and score and delta Rank1-Rank2 score thresholds optimized to yield a PSM-level FDR estimate for precursor charges 2 through 4 of <1.0% for each precursor charge state in each LC-MS/MS run. To achieve reasonable statistics for precursor charges 5-6, thresholds were optimized to yield a PSM-level FDR estimate of <0.5% across all LC runs per experiment (instead of per each run), since many fewer spectra are generated for the higher charge states. In step 2, protein-polishing autovalidation was applied to each experiment to further filter the PSMs using a target protein-level FDR threshold of zero, the protein grouping method expand subgroups, top uses shared (SGT) with an absolute minimum protein score of 9. After assembling protein groups from the autovalidated PSMs, protein polishing determined the maximum protein level score of a protein subgroup that consisted entirely of distinct peptides estimated to be false-positive identifications (PSMs with negative delta forward-reverse scores); B721: 11.6, GBM: 10.5. PSMs were removed from the set obtained in the initial peptide-level autovalidation step if they contributed to protein subgroups that had protein scores below the maximum false-positive protein score. In Spectrum Mill the protein score was the sum of the scores of distinct peptides. When a peptide sequence of >8 residues was shared by multiple protein entries in the sequence database, the proteins were grouped together. In some cases there were unshared peptides that uniquely represent a subgroup, i.e. lower scoring member of the group, typically isoforms, family members, or different species. As a consequence of these two peptide and protein level steps, each identified protein subgroup was comprised of multiple peptides, unless a single excellent scoring peptide was the sole match.

In the cases where a spectrum could be matched to multiple peptide sequences from different ORFs, the same decision tree was followed for the whole proteome analysis as for the MHC I described above.

### Absolute Translation Level Estimation

Our improved translation quantification based on Ribo-seq reads incorporates multi-mapping information and translated frame information. To account for multi-mapping, reads were scaled based on their number of alignments: For example, if a read maps to a 5 different ORFs, it will contribute 0.2 at each location. Using the offset-corrected SAM file generated by RibORF (described above), and given that Applicants know the translated frame identified by Ribo-seq, Applicants counted the total number of multimapping-adjusted reads that are in-frame for each ORF in nuORFdb's BED12 file using a custom script, and calculated TPM using those read counts and the ORF length. The Python script is provided.

Peptide sequence correlation, clustering and visualization Peptide distance computation and visualization were performed as before (1), Sarkizova et al, 2019). Briefly, peptide distances were defined as:

$$D ( A , {s}_{1} , {s}_{2} ) = \frac{1}{L} {\sum }_{i = 1}^{L} distPMBEC ( {s}_{1 i} , {s}_{2 i} ) * ( 1 - {H}_{Ai} )$$

where A is the allele; s₁ and s₂ are peptide sequences; L is the length of the peptide sequences, n∈{8,9,10,11}; H is the entropy of the amino acid residues at each position in the peptide, distPMBEC=maxPMBEC −PMBEC is a 20×20 matrix of residue dissimilarities derived from a pre-computed matrix of residue similarities biased by their HLA binding properties (16). For each allele, peptide distances between every pair of peptides in the MS datasets was computed and the pairwise distance matrices were reduced to two dimensions with non-metric multidimensional scaling (NMDS) (nmds( ) function from ecodist R package).

Peptide sequence motif correlation (for FIG. 33F) was calculated per allele using all detected 9AA peptides. For each peptide, the frequency of each amino acid at each position was calculated to generate a vector of 180 features long. Using these vectors, the position entropy weighted correlation was found between nuORF peptides and all annotated peptides, or between 10,000 random subsets of annotated peptides the same size as the nuORF set and all annotated peptides (minus the subset). Correlations were calculated for all 92 measured alleles independently

### Whole Genome Sequencing and Analysis

PCR-free Whole Genome Sequencing (WGS) was performed on cultured melanoma patient 11 cells and matched healthy PBMCs at the Broad Genomics Platform. Libraries were prepared using the Kapa Biosciences HyperPrep library construction kit, and sequenced to 60× coverage (Illumina 2×150 bp reads, NovaSeq). Cancer-specific variants were identified using GATK Best Practices (GATK v3.x nightly-2017-09-30) (17) and Strelka2 v2.8.4 (18). In particular, Applicants first aligned sequenced reads to human genome reference assembly GRCh37 using BWA-MEM (19) v0.7.15-r1140 with default parameters. Applicants then sort aligned reads by coordinates and removed PCR duplicates using Picard tool v2.12.1 [http://broadinstitute.github.io/picard/]. Next, Applicants applied base quality score recalibration to the de-duplicated BAM files using GATK. The recalibrated BAM files were used as inputs for both GATK and Strelka2 for calling somatic variants. For GATK, Applicants followed best practices and used MuTect2 with —dbsnp set to dbSNP build 138 [https://www.ncbi.nlm.nih.gov/snp/] and —cosmic set to Cosmic v82 [https://cosmic-blog.sanger.ac.uk/cosmic-release-v82/]. For Strelka2, Applicants first ran Manta (20) v1.2.1 to detect structural variants and indels as recommended by Strelka2 user guide [https://github.com/Illumina/strelka/blob/v2.9.x/docs/userGuide/README.md]. Applicants then ran Strelka2 with —indelCandidates option set to Manta outputs and other options set to default values. Applicants merged variants called using GATK and Strelka2 together.

### Variant Analysis, Read Coverage, and Neoantigen Predictions

To derive ORFs containing cancer-specific variants identified by WGS, variants that were found within the reference transcripts used in the study were selected using bedtools intersect (21) v2.25.0 of the BED12 file of transcripts with the VCF file of variants. Variants were then incorporated into the transcript sequences, and ORFs were re-derived based on the predicted start codon in nuORFdb and the first in-frame stop codon.

To determine Ribo-seq read coverage and nucleotide identity at the SNV sites, pysam pileup (v0.14.1) was used. To obtain read coverage of indels, bowtie (1.2.2)-m 1-v 0 was used to align raw sequencing reads (after adapter trimming) to a custom FASTA reference that included matched wild-type and indel-containing regions. No multi-mapping reads or mismatches were allowed, such that only variant- or wild-type supporting reads were retained.

Variants supported by at least 9 Ribo-seq reads and >15% of total reads at the locus were used for neoantigen predictions. To obtain potential neoantigens from the mutated variants, all possible 9- and 10-amino acid long peptides were derived from wild-type and variant-containing proteins in nuORFdb. Peptides unique to the variant-containing proteins were retained as potential neoantigens. NetMHCpan v4.0 was used to predict neoantigen binding affinities to HLA alleles (22). Indels were visualized in IGV (23) to identify in-frame Ribo-seq reads supporting the translation of indel-generated frame-shifted ORFs and wild-type ORFs.

Identification of Tissue-Specific or Tissue-Enriched nuORFs

For the TCGA analysis, Applicants included 473 available skin cutaneous melanoma (SKCM) samples and 172 glioblastoma multiforme (GBM) samples. For GTEx (24), Applicants randomly selected 10 samples from each tissue. For CLL, available data from 390 CLLs and 21 B-cell samples from healthy donors were included. These comprise two cohorts: 106 CLL and 12 healthy samples from DFCI/Broad Institute (25) and 284 CLL and 9 healthy samples from Spanish ICGC studies (26, 27). FASTQ files from all cohorts were aligned using STAR v2.6.1d (7) to the reference human genome GRCh37, using the transcriptome annotation combing GENCODE and MiTranscriptome, as used for Ribo-seq based ORF detection described above. Expression at the gene-level was quantified using RNA-SeQC v2.3.3, and expression at the isoform level was quantified using RSEM v1.3.1 (28). The parameters used for all components of this pipeline are described at https://github.com/broadinstitute/gtex-pipeline/blob/v9/TOPMed RNAseq_pipeline.md. Expression quantification (TPM) across transcript isoforms (CLL, B cells), (GTEx, TCGA) was also performed.

Identifying Cancer-Enriched nuORFs Based on MHC I IP LC-MS/MS

Applicants generated a list of 381 nuORFs detected by LC-MS/MS in the MHC I immunopeptidomes of the 6 melanoma samples Applicants analyzed. Applicants rank ordered nuORFs by mean expression of the parent transcript across all GTEx samples, excluding the testis, and selected 38 nuORFs with mean expression in the lowest 10% enriching for those not expressed or lowly expressed in healthy tissues. Applicants further filtered them based on the nuORF parent transcript expression across 473 melanoma samples in the TCGA, retaining 6 nuORFs where at least 5% of TCGA samples had expression 2-fold or greater than the highest level detected in any GTEx sample.

Identifying Cancer-Specific nuORFs Based on Ribo-Seq

Based on the Ribo-seq translation levels (TPM), Applicants selected nuORFs with TPM>0 across all in-group samples (all CLL samples/all GBM samples/all MEL samples) and TPM=0 in the rest of the Ribo-seq samples profiled. Applicants retained those nuORFs with parent transcript TPM <1 across healthy tissues in GTEx, excluding the testis.

### Statistical Analyses

FIG. 33A, 33C: In the comparison of the MS/MS spectrum scores calculated by Spectrum Mill (FIG. 33A) as well as the translation levels of ORFs (FIG. 33C), the sample sizes were very large, thus the t-tests showed significance, yet the effect size is small, as shown by the confidence intervals calculated using linear regression by the python package statsmodels.regression.linear_model.OLS.

FIG. 33D, 39D: Retention time vs. predicted hydrophobicity: Lowess was fit to the annotated peptide retention time and hydrophobicity values using the python package sm.nonparametric.lowess. Residuals between annotated peptide identifications to the lowess fit and residuals between nuORF peptide identifications to the lowess fit were computed and compared with rank sum test in python using scipy.stats.ranksums.

FIG. 33H: The lengths of detected Canonical ORFs were compared to the lengths of the detected ORFs in each of the shown categories using a t-test with unequal variance in python using scipy.stats.ttest_ind.

FIG. 33M, 42H: The cumulative distribution functions (CDFs) for length or translation level (TPM) of annotated ORFs or nuORFs detected in the MHC-I immunopeptidome or in the whole proteome, compared with a KS test using the python scipy.stats.kstest.

FIG. 34G: Given the variable number of known and B721 matched HLA alleles in cancer patients, Applicants simulated the % overlap with variable numbers of alleles matching. All overlaps were measured between 6 B721 alleles randomly sampled from the measured 92 alleles, with a fixed number of type matched alleles. These simulations were calculated for both annotated and nuORF peptides. Applicants then calculated a linear regression between the number of matched alleles and the median % overlap for each cancer sample for both annotated and nuORF.

FIG. 35E: Using netMHCpan v4.0, Applicants predicted the rate of strong binders (predicted binding <500 nM) for all high confidence SNVs that also showed strong Ribo-seq support, with at least 9 Ribo-seq reads and 15% of all reads supporting the SNV. Applicants compared the strong binder rate for annotated- and nuORF-derived mutations using a t-test and calculated confidence intervals using linear regression.

FIG. 35I: For each nuORF identified as being cancer type specific using ribosome profiling data and low GTEx expression, Applicants compared the expression in TCGA for the associated cancer type to other cancer types and to GTEx, with a rank sum test in python scipy.stats.ranksums. Higher expression in respective TCGA samples was indicated on the far right of 35I and the percent of predicted nuORFs significantly upregulated is shown in 35J.

FIG. 33K, 42C, 42F: Applicants tested for enrichment or depletion of nuORF types in Whole Proteome or cancer samples by generating a % detected distribution for each nuORF type by randomly sampling 1 to 6 B721 alleles from the 92 measured, and reporting the % of nuORFs of each type. Applicants then calculated the p-value for enrichment or depletion as the ratio of the simulated distribution greater than or less than the observed, respectively. To test for overall enrichment or depletion in cancers, Applicants used a t-test to compare the observed p-values to a normal distribution.

### Data and Code Availability

Python scripts and Jupiter notebooks used in the analysis will be deposited in GitHub Mass spectrometry data

The original mass spectra for immunopeptidomes of 2 melanoma patient-derived cell lines and the full proteome of a glioblastoma patient-derived cell line, tables of peptide spectrum matches for all experiments, and the protein sequence databases used for searches have been deposited in the public proteomics repository MassIVE (https://massive.ucsd.edu) and are accessible at ftp://MSV00008??@massive.ucsd.edu.

Original mass spectrometry data for the previously published mono-allelic immunopeptidomes, B721.221 cell line full proteome, and patient-derived cell line immunopeptidomes are accessible at ftp://massive.ucsd. edu/MSV000080527, ftp://massive.ucsd. edu/MS V000084172, and ftp://massive.ucsd. edu/MSV000084442. B721.221 RNA seq data for HLA-C(C*04:01, C*07:01) is deposited under GEO: GSE131267. Melanoma RNA-seq data are deposited in dbGaP (https://www.ncbi.nlm.nih.gov/projects/gap/cgi-bin/study.cgi?study_id=phs001451.v1.p1(4)). Glioblastoma bulk RNA-seq data are available through dbGaP (https://www.ncbi.nlm.nih.gov/gap) with accession number phs001519.v1.p1(5). All other data are available from the corresponding authors upon reasonable request.

### REFERENCES

1. J. G. Abelin, D. B. Keskin, S. Sarkizova, C. R. Hartigan, W. Zhang, J. Sidney, J. Stevens, W. Lane, G. L. Zhang, T. M. Eisenhaure, K. R. Clauser, N. Hacohen, M. S.
Rooney, S. A. Carr, C. J. Wu, Mass Spectrometry Profiling of HLA-Associated Peptidomes in Mono-allelic Cells Enables More Accurate Epitope Prediction. Immunity. 46, 315-326 (2017).
2. S. Sarkizova, S. Klaeger, P. Le, L. Li, G. Oliveira, H. Keshishian, C. Hartigan, W. Zhang, D. Braun, P. Bachireddy, K. Ligon, I. Zervantonakis, J. Rosenbluth, T. Ouspenskaia, T. Law, S. Justesen, J. Stevens, W. Lane, T. Eisenhaure, G. L. Zhang, K. Clauser, N. Hacohen, S. Carr, D. Keskin, A large peptidome dataset improves HLA class I epitope prediction across most of the human population. Nat. Biotechnol. (in press).
3. J. G. Abelin, D. B. Keskin, S. Sarkizova, C. R. Hartigan, W. Zhang, J. Sidney, J. Stevens, W. Lane, G. L. Zhang, T. M. Eisenhaure, K. R. Clauser, N. Hacohen, M. S. Rooney, S. A. Carr, C. J. Wu, Mass Spectrometry Profiling of HLA-Associated Peptidomes in Mono-allelic Cells Enables More Accurate Epitope Prediction. Immunity. 46, 315-326 (2017).
4. P. A. Ott, Z. Hu, D. B. Keskin, S. A. Shukla, J. Sun, D. J. Bozym, W. Zhang, A. Luoma, A. Giobbie-Hurder, L. Peter, C. Chen, O. Olive, T. A. Carter, S. Li, D. J. Lieb, T. Eisenhaure, E. Gjini, J. Stevens, W. J. Lane, I. Javeri, K. Nellaiappan, A. M. Salazar, H. Daley, M. Seaman, E. I. Buchbinder, C. H. Yoon, M. Harden, N. Lennon, S. Gabriel, S. J. Rodig, D. H. Barouch, J. C. Aster, G. Getz, K. Wucherpfennig, D. Neuberg, J. Ritz, E. S. Lander, E. F. Fritsch, N. Hacohen, C. J. Wu, An immunogenic personal neoantigen vaccine for patients with melanoma. Nature. 547, 217-221 (2017).
5. D. B. Keskin, A. J. Anandappa, J. Sun, I. Tirosh, N. D. Mathewson, S. Li, G. Oliveira, A. Giobbie-Hurder, K. Felt, E. Gjini, S. A. Shukla, Z. Hu, L. Li, P. M. Le, R. L. Allesøe, A. R. Richman, M. S. Kowalczyk, S. Abdelrahman, J. E. Geduldig, S. Charbonneau, K. Pelton, J. B. Iorgulescu, L. Elagina, W. Zhang, O. Olive, C. McCluskey, L. R. Olsen, J. Stevens, W. J. Lane, A. M. Salazar, H. Daley, P. Y. Wen, E. A. Chiocca, M. Harden, N. J. Lennon, S. Gabriel, G. Getz, E. S. Lander, A. Regev, J. Ritz, D. Neuberg, S. J. Rodig, K. L. Ligon, M. L. Suva, K. W. Wucherpfennig, N. Hacohen, E. F. Fritsch, K. J. Livak, P. A. Ott, C. J. Wu, D. A. Reardon, Neoantigen vaccine generates intratumoral T cell responses in phase Ib glioblastoma trial. Nature. 565, 234-239 (2019).
6. B. Langmead, C. Trapnell, M. Pop, S. L. Salzberg, Ultrafast and memory-efficient alignment of short DNA sequences to the human genome. Genome Biol. 10, R25 (2009).
7. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics. 29, 15-21 (2013).
8. M. K. Iyer, Y. S. Niknafs, R. Malik, U. Singhal, A. Sahu, Y. Hosono, T. R. Barrette, J. R. Prensner, J. R. Evans, S. Zhao, A. Poliakov, X. Cao, S. M. Dhanasekaran, Y. M. Wu, D. R. Robinson, D. G. Beer, F. Y. Feng, H. K. Iyer, A. M. Chinnaiyan, The landscape of long noncoding RNAs in the human transcriptome. Nat. Genet. 47, 199-208 (2015).
9. Z. Ji, R. Song, A. Regev, K. Struhl, Many lncRNAs, 5′UTRs, and pseudogenes are translated and some are likely to express functional proteins. Elife. 4 (2015), doi:10.7554/eLife.08890.
10. F. Erhard, A. Halenius, C. Zimmermann, A. L'Hernault, D. J. Kowalewski, M. P. Weekes, S. Stevanovic, R. Zimmer, L. Dolken, Improved Ribo-seq enables identification of cryptic translation events. Nat. Methods (2018), doi:10.1038/nmeth.4631.
11. B. Malone, I. Atanassov, F. Aeschimann, X. Li, H. Grosshans, C. Dieterich, Bayesian prediction of RNA translation from ribosome profiling. Nucleic Acids Res. 45, 2960-2972 (2017).
12. M. Bassani-Sternberg, E. Bräunlein, R. Klar, T. Engleitner, P. Sinitcyn, S. Audehm, M. Straub, J. Weber, J. Slotta-Huspenina, K. Specht, M. E. Martignoni, A. Werner, R. Hein, D. H Busch, C. Peschel, R. Rad, J. Cox, M. Mann, A. M. Krackhardt, Direct identification of clinically relevant neoepitopes presented on native human melanoma tissue by mass spectrometry. Nat. Commun. 7, 13404 (2016).
13. P. Faridi, C. Li, S. H. Ramarathinam, J. P. Vivian, P. T. Illing, N. A. Mifsud, R. Ayala, J. Song, L. J. Gearing, P. J. Hertzog, N. Ternette, J. Rossjohn, N. P. Croft, A. W. Purcell, A subset of HLA-I peptides are not genomically templated: Evidence for cis- and trans-spliced peptide ligands. Sci Immunol. 3 (2018), doi:10.1126/sciimmunol.aar3947.
14. O. V. Krokhin, S. Ying, R. Craig, V. Spicer, W. Ens, K. G. Standing, R. C. Beavis, J. A. Wilkins, in Proceedings of the 52-th ASMS Conference on Mass Spectrometry and Allied Topics. Nashville, Tenn., USA (2004; https://www.researchgate.net/profile/John_Wilkins/publication/265422254_New_sequence-specific_correction_factors_for_prediction_of_peptide_retention_in_RP-HPLC_application_to_protein_identification_by_off-line_HPLC-MALDI- MS/links/5730cf8308aed286ca0dc01b.pdf).
15. P. Mertins, L. C. Tang, K. Krug, D. J. Clark, M. A. Gritsenko, L. Chen, K. R. Clauser, T. R. Clauss, P. Shah, M. A. Gillette, V. A. Petyuk, S. N. Thomas, D. R. Mani, F. Mundt, R. J. Moore, Y. Hu, R. Zhao, M. Schnaubelt, H. Keshishian, M. E. Monroe, Z. Zhang, N. D. Udeshi, D. Mani, S. R. Davies, R. R. Townsend, D. W. Chan, R. D. Smith, H. Zhang, T. Liu, S. A. Carr, Reproducible workflow for multiplexed deep-scale proteome and phosphoproteome analysis of tumor tissues by liquid chromatography-mass spectrometry. Nat. Protoc. 13, 1632-1661 (2018).
16. Y. Kim, J. Sidney, C. Pinilla, A. Sette, B. Peters, Derivation of an amino acid similarity matrix for peptide: MHC binding and its application as a Bayesian prior. BMC Bioinformatics. 10, 394 (2009).
17. A. Bateman, W. R. Pearson, L. D. Stein, G. D. Stormo, J. R. Yates III, Eds., in Current Protocols in Bioinformatics (John Wiley & Sons, Inc., Hoboken, N.J., USA, 2002), vol. 467, pp. 11.10.1-11.10.33.
18. S. Kim, K. Scheffler, A. L. Halpern, M. A. Bekritsky, E. Noh, M. Milberg, X. Chen, Y. Kim, D. Beyter, P. Krusche, C. T. Saunders, Strelka2: fast and accurate calling of germline and somatic variants. Nat. Methods. 15, 591-594 (2018).
19. H. Li, Aligning sequence reads, clone sequences and assembly contigs with BWA-MEM. arXiv [q-bio.GN] (2013), (available at http://arxiv.org/abs/1303.3997).
20. X. Chen, O. Schulz-Trieglaff, R. Shaw, B. Barnes, F. Schlesinger, M. Källberg, A. J. Cox, S. Kruglyak, C. T. Saunders, Manta: rapid detection of structural variants and indels for germline and cancer sequencing applications. Bioinformatics. 32, 1220-1222 (2016).
21. A. R. Quinlan, I. M. Hall, BEDTools: a flexible suite of utilities for comparing genomic features. Bioinformatics. 26, 841-842 (2010).
22. V. Jurtz, S. Paul, M. Andreatta, P. Marcatili, B. Peters, M. Nielsen, NetMHCpan-4.0: Improved Peptide-WIC Class I Interaction Predictions Integrating Eluted Ligand and Peptide Binding Affinity Data. J. Immunol. 199, 3360-3368 (2017).
23. J. T. Robinson, H. Thorvaldsdóttir, W. Winckler, M. Guttman, E. S. Lander, G. Getz, J. P. Mesirov, Integrative genomics viewer. Nat. Biotechnol. 29, 24-26 (2011).
24. GTEx Consortium, Laboratory, Data Analysis & Coordinating Center (LDACC)—Analysis Working Group, Statistical Methods groups—Analysis Working Group, Enhancing GTEx (eGTEx) groups, NIH Common Fund, NIH/NCI, NIH/NHGRI, NIH/NIDA, Biospecimen Collection Source Site—NDRI, Biospecimen Collection Source Site—RPCI, Biospecimen Core Resource—VARI, Brain Bank Repository—University of Miami Brain Endowment Bank, Leidos Biomedical—Project Management, ELSI Study, Genome Browser Data Integration & Visualization—EBI, Genome Browser Data Integration & Visualization—UCSC Genomics Institute, University of California Santa Cruz, Lead analysts:, Laboratory, Data Analysis & Coordinating Center (LDACC):, NIH program management:, Biospecimen collection:, Pathology:, eQTL manuscript working group:, A. Battle, C. D. Brown, B. E. Engelhardt, S. B. Montgomery, Genetic effects on gene expression across human tissues. Nature. 550, 204-213 (2017).
25. D. A. Landau, E. Tausch, A. N. Taylor-Weiner, C. Stewart, J. G. Reiter, J. Bahlo, S. Kluth, I. Bozic, M. Lawrence, S. Böttcher, S. L. Carter, K. Cibulskis, D. Mertens, C. L. Sougnez, M. Rosenberg, J. M. Hess, J. Edelmann, S. Kless, M. Kneba, M. Ritgen, A. Fink, K. Fischer, S. Gabriel, E. S. Lander, M. A. Nowak, H. Döhner, M. Hallek, D. Neuberg, G. Getz, S. Stilgenbauer, C. J. Wu, Mutations driving CLL and their evolution in progression and relapse. Nature. 526, 525-530 (2015).
26. P. G. Ferreira, P. Jares, D. Rico, G. Gómez-López, A. Martinez-Trillos, N. Villamor, S. Ecker, A. González-Pérez, D. G. Knowles, J. Monlong, R. Johnson, V. Quesada, S. Djebali, P. Papasaikas, M. López-Guerra, D. Colomer, C. Royo, M. Cazorla, M. Pinyol, G. Clot, M. Aymerich, M. Rozman, M. Kulis, D. Tamborero, A. Gouin, J. Blanc, M. Gut, I. Gut, X. S. Puente, D. G. Pisano, J. I. Martin-Subero, N. López-Bigas, A. López-Guillermo, A. Valencia, C. López-Otin, E. Campo, R. Guigó, Transcriptome characterization by RNA sequencing identifies a major molecular and clinical subdivision in chronic lymphocytic leukemia. Genome Res. 24, 212-226 (2014).
27. X. S. Puente, S. Beà, R. Valdes-Mas, N. Villamor, J. Gutierrez-Abril, J. I. Martin-Subero, M. Munar, C. Rubio-Pérez, P. Jares, M. Aymerich, T. Baumann, R. Beekman, L. Belver, A. Carrio, G. Castellano, G. Clot, E. Colado, D. Colomer, D. Costa, J. Delgado, A. Enjuanes, X. Estivill, A. A. Ferrando, J. L. Gelpí, B. González, S. González, M. Gonzalez, M. Gut, J. M. Hernández-Rivas, M. López-Guerra, D. Martín-García, A. Navarro, P. Nicolás, M. Orozco, Á. R. Payer, M. Pinyol, D. G. Pisano, D. A. Puente, A. C. Queirós, V. Quesada, C. M. Romeo-Casabona, C. Royo, R. Royo, M. Rozman, N. Russiñol, I. Salaverría, K. Stamatopoulos, H. G. Stunnenberg, D. Tamborero, M. J. Terol, A. Valencia, N. López-Bigas, D. Torrents, I. Gut, A. López-Guillermo, C. López-Otin, E. Campo, Non-coding recurrent mutations in chronic lymphocytic leukaemia. Nature. 526, 519-524 (2015).
28. B. Li, C. N. Dewey, RSEM: accurate transcript quantification from RNA-Seq data with or without a reference genome. BMC Bioinformatics. 12, 323 (2011).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A polypeptide comprising one or more neoantigens from Table 1-3D.

2. The polypeptide of claim 1, comprising 2 or more neoantigens.

3. The polypeptide of claim 2, wherein the 2 or more neoantigens are linked directly together.

4. The polypeptide of any one of the preceding claims further comprising a T cell enhancer amino acid sequence.

5. The polypeptide of claim 4, wherein the T cell enhancer amino acid is selected from the group consisting of an invariant chain; a leader sequence of tissue-type plasminogen activator; a PEST sequence, a cyclin destruction box; a ubiquitination signal; and a SUMOylation signal.

6. A polynucleotide encoding the polypeptide of any one of claims 1 to 5.

7. A vector comprising the polynucleotide of claim 6.

8. A vector system comprising one or more expression vectors of claim 7, wherein each expression vector is selected from the group consisting of a plasmid, a cosmid, a RNA, a RNA formulated in a particle, a self-amplifying RNA (SAM), a SAM formulated in a particle, or a viral vector.

9. The vector system of claim 8, wherein the particle is a liposomal particle.

10. The vector system of claim 8, wherein the vector is a viral vector.

11. The vector system of claim 10, wherein the viral vector is an alpha virus vector, a Venezuelan equine encephalitis (VEE) virus vector, a sindbis virus vector, a semliki forest virus vector, a simian or human cytomegalovirus vector, a lymphocyte choriomenigitis virus vector, a retroviral vector, a lentiviral vector, an adenovirus vector, or combination thereof.

12. A composition comprising the polypeptide of any one of claims 1 to 5.

13. A composition comprising the vector claim 7.

14. A composition comprising the vector system of any one of claims 8 to 11.

15. The composition of any one of claims 12 to 14, further comprising at least one modulator of a checkpoint molecule or an immunomodulator, or a nucleic acid encoding the modulator or immunomodulator, or a vector comprising the nucleic acid encoding the modulator or immunomodulator for use in preventing or treating a proliferative disease in a subject.

16. The composition of claim 15, wherein the modulator of a checkpoint molecule is selected from the group consisting;
a. an agonist of a tumor necrosis factor receptor superfamily member, preferably of CD27, CD40, OX40, GITR, or CD137; and/or
b. an antagonist of PD-1, PD-L1, CD274, A2AR, B7-H3, B7-H4, BTLA<CTLA-4, IDO, KIR, LAG3, TIM-3, VISTA, or an antagonist of a B7-CD28 superfamily member, preferably of CD28 or ICOS or an antagonist of a ligand thereof; and/or
c. the immunomodulator is a T cell growth factor, preferably IL-2, IL-12, or IL-15.

17. The composition of any one of claims 12 to 16, further comprising one or more adjuvants.

18. A method for identifying neoantigens, comprising:
a) performing Ribosomal profiling (Ribo-seq) on a sample or set of samples;
b) generating a novel untranslated open reading frame (nuORF) database comprising predicted nuORFs by conducting hierarchical ORF prediction on the Ribo-seq data generated in (a); and
c) generating a final set of neoantigens by searching the nuORF database for predicted nuORFs in the nuORF database matching data in a WIC I immunopeptidome data set, the identified presented nuORFs comprising the final neoantigen set.

19. The method of claim 18, further comprising searching an annotated proteome database for ORFs in the annotated proteome database matching data in the WIC I immunopeptidome dataset.

20. The method of claim 19, further comprising selecting presented nuORFs identified in the nuORF database but not the annotated proteome database to generate the final set of neoantigens.

21. The method of any one of claims 18 to 20, wherein WIC I immunopeptidome data is obtained on biological sampled from a subject to be treated.

22. The method of any one of claims 18 to 20, wherein the Ribo-seq data is obtain from a biological sample from a subject to be treated.

23. The method of claim 7, wherein the immunopeptidome data is mass spectroscopy data.

24. A polypeptide comprising one or more neoantigens identified by the method of claim 18.

25. A polynucleotide encoding the polypeptide of claim 24.

26. A vector comprising the polynucleotide of claim 25.

27. A vector system comprising one or more vectors of claim 26.

28. A composition comprise the polypeptide of claim 24, the vector of claim 26, or the vector system of claim 27

29. A method of identifying subject-specific T cell receptor (TCR) pairs suitable for subject-specific cancer therapy, the method comprising:
isolating from the subject a population comprising T cells;
determining by single cell sequencing the sequences encoding the TCR pairs on individual cells in the population isolated in (a);
transfecting or transducing T cell lines deficient in endogenous TCRs with the sequences encoding individual TCR pairs determined in (b);
using the T cell lines from (c) to assay binding of the subject specific TCR pairs to subject specific neoepitopes and selecting the TCR pairs that bind to subject-specific neoepitopes.

30. The method of claim 29, wherein the subject specific neoepitopes are expressed on HLA molecules on a cell.

31. The method of claim 30, wherein the cells are antigen presenting cells.

32. The method of claim 29, wherein the binding of the T cells to the neoepitopes activates a reporter gene.

33. The method of claim 29, wherein the neoepitopes are present in tetramers.

34. The method of claim 29, wherein the neoepitopes are nuORFs.

35. The method of any of claims 29-34, wherein the sample or set of samples is subject-specific, tissue specific or disorder-specific, or disease-specific.

36. The method of claim 35, wherein the disease is or disorder is genetic, pathogenic or cancer.

37. A method of generating antibodies comprising administering the polypeptide of any one of claim 1-5 or 24, the vector of any one of claim 7 or 26, or the vector system of any one of claim 8-11 or 27, or the composition of any one of claim 12-17 or 28 to the immune system, or a component thereof, of the subject.

38. The method of claim 37, wherein the component is a B cell.

39. A method of treatment comprising administering the neoantigen composition of any of the preceding claims to a subject with a disease.

40. A method for identifying patient specific neoantigens comprising:
performing Ribosomal profiling (Ribo-seq) on a patient specific tumor sample and a non-tumor sample from the patient; and
identifying nuORFs specific for the tumor sample.

41. The method of claim 40, further comprising identifying T cells obtained from the patient specific for one or more of the identified neoantigens.

42. The method of claim 41, further comprising expanding T cells specific for the one or more of the identified neoantigens.

43. A T cell specific for a neoantigen identified by the method of any one of claims 18 to 23.

44. The T cell of claim 43, wherein the T cell is obtained from PBMCs from the patient.
